(19) United States
(12) Patent Application Publication
(10) Pub. No.: US 2024/0294542 A1
(43) Pub. Date: Sep. 5, 2024

(54) NLRP3 INFLAMMASOME INHIBITOR AND APPLICATION THEREOF

(71) Applicant: TransThera Sciences (Nanjing), Inc., Jiangbei New Area Nanjing, Jiangsu (CN)

(72) Inventors: Lin Li, Jiangbei New Area Nanjing, Jiangsu (CN); Frank Wu, Jiangbei New Area Nanjing, Jiangsu (CN)

(21) Appl. No.: 18/566,865

(22) PCT Filed: Jun. 2, 2022
(86) PCT No.: PCT/CN2022/096928

§ 371 Date: Dec. 4, 2023

(30) Foreign Application Priority Data
Jun. 5, 2021 (CN) 202110629439.0
Jul. 26, 2021 (CN) 202110843496.9
Aug. 13, 2021 (CN) 202110940013.7
Aug. 13, 2021 (CN) 202110940018.X
Jan. 7, 2022 (CN) 202210015181.X
Jan. 7, 2022 (CN) 202210015699.3

(51) Int. Cl. C07D 495/04; A61K 31/502; A61K 31/5025; A61K 31/5377; C07D 237/34; C07D 401/12; C07D 401/14; C07D 405/14; C07D 413/14; C07D 471/04; C07D 487/04; C07D 491/048; C07D 491/18

(52) U.S. Cl. CPC C07D 495/04; A61K 31/502; A61K 31/5025; A61K 31/5377; C07D 237/34; C07D 401/12; C07D 401/14; C07D 405/14; C07D 413/14; C07D 471/04; C07D 487/04; C07D 491/048; C07D 491/18

(57) ABSTRACT

The present invention belongs to the technical field of medicines, relates to an NLRP3 inflammasome inhibitor and use thereof, and particularly relates to a compound of general formula (I), or a pharmaceutically acceptable salt, a stereoisomer or a tautomer thereof, wherein each group is defined in the specification. Researches show that the compound of general formula (I), or pharmaceutically acceptable salt, the stereoisomer or the tautomer thereof has relatively high biological activity on NLRP3 inflammasomes and has an important clinical development value for the treatment of NLRP3-associated diseases.

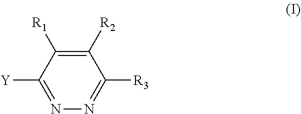

### CROSS-REFERENCE TO RELATED APPLICATIONS

The present invention claims priority to the following invention patent applications:
Invention Patent Application No. 202110629439.0 filed with China National Intellectual Property Administration on Jun. 5, 2021 and entitled “NLRP3 INFLAMMASOME INHIBITOR AND USE THEREOF”;
Invention Patent Application No. 202110843496.9 filed with China National Intellectual Property Administration on Jul. 26, 2021 and entitled “NLRP3 INFLAMMASOME INHIBITOR AND USE THEREOF”;
Invention Patent Application No. 202110940013.7 filed with China National Intellectual Property Administration on Aug. 13, 2021 and entitled “NLRP3 INFLAMMASOME INHIBITOR AND USE THEREOF”;
Invention Patent Application No. 202110940018.X filed with China National Intellectual Property Administration on Aug. 13, 2021 and entitled “NLRP3 INFLAMMASOME INHIBITOR AND USE THEREOF”;
Invention Patent Application No. 202210015181.X filed with China National Intellectual Property Administration on Jan. 7, 2022 and entitled “NLRP3 INFLAMMASOME INHIBITOR AND USE THEREOF”;
Invention Patent Application No. 202210015699.3 filed with China National Intellectual Property Administration on Jan. 7, 2022 and entitled “NLRP3 INFLAMMASOME INHIBITOR AND USE THEREOF”,
which are all incorporated herein in their entireties.

### TECHNICAL FIELD

The present invention belongs to the technical field of medicines, and particularly relates to an NLRP3 inflammasome inhibitor and use thereof.

### BACKGROUND

Nucleotide-binding oligomerization domain (NOD)-like receptor protein 3 (NLRP3) belongs to the NOD-like receptors (NLRs) family, also known as “pyrin domain-containing protein 3”. NLRP3 contains three modules which are a pyrin domain (PYD), a nucleotide-binding site domain (NBD), and a leucine-rich repeat (LRR). Upon receiving a stimulus from a sterile inflammatory risk signal, NLRP3 interacts with adaptor apoptosis-associated speck-like protein containing a CARD (ASC) and pro-caspase 1 to form an NLRP3 inflammasome. Activation of the NLRP3 inflammasome results in the release of interleukin-1β (IL-1β) and interleukin-18 (IL-18).

Activation of NLRP3 inflammasome usually requires two steps. The first step involves a priming signal, where Toll-like receptors recognize pathogen-associated molecular patterns (PAMPs) or damage-associated molecular patterns (DAMPs) which in turn transmit a signal into the cells to mediate the activation of the NF-κB signaling pathway, thereby up-regulating the transcription levels of NLRP3 inflammasome-associated components including inactive NLRP3 and pro-IL-1β. The second step involves an activation signal; after a P2X7 receptor and the like receive signal stimulation of ATP, nigericin and the like, an NLRP3 monomer is oligomerized to form an NLRP3 oligomer, and then ASC and pro-caspase 1 are recruited to assemble into an NLRP3 inflammasome complex. This triggers the conversion of pro-caspase 1 to caspase 1, and also the production and secretion of mature IL-1β and IL-1β.

Activation of NLRP3 inflammasome is associated with various diseases, for example, autoinflammatory fever syndromes such as cryo-pyrin-associated periodic syndromes (CAPS), sickle cell disease, systemic lupus erythematosus (SLE), chronic liver disease, nonalcoholic steatohepatitis (NASH), gout, pseudogout (chondrocalcinosis), type I and type II diabetes and related complications (e.g., nephropathy, retinopathy), neuroinflammation-related disorders (e.g., multiple sclerosis, brain infection, acute injury, neurodegenerative disease, Alzheimer's disease), atherosclerosis and cardiovascular risk (e.g., hypertension), hidradenitis suppurativa, wound healing and scarring, and cancer (e.g., colorectal cancer, lung cancer, myeloproliferative tumors, leukemia, myelodysplastic syndrome (MDS), myelofibrosis). Most treatment methods include symptomatic treatment, slowing of the progression of the diseases/disorders, and surgeries as the last treatment means.

Currently, as the varieties of NLRP3 inflammasome inhibitors are few, the development of an NLRP3 inflammasome inhibitor with relatively high activity and better druggability becomes clinical requirements.

### SUMMARY

The present invention researches the following compound, or a pharmaceutically acceptable salt, a stereoisomer or a tautomer thereof, and discovers that the compound, or the pharmaceutically acceptable salt, the stereoisomer or the tautomer thereof has relatively high biological activity on an NLRP3 inflammasome and has important clinical development value for treating NLRP3-associated diseases.

In order to achieve the above objective, the present invention provides the following technical solutions:

The technical solutions of the present invention include a compound of general formula (I), or a pharmaceutically acceptable salt, a stereoisomer or a tautomer thereof:

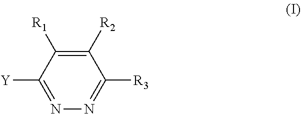

wherein,
R₁ is selected from hydrogen, hydroxyl, amino, carboxyl, cyano, nitro, halogen, C₁₋₆ alkyl, haloC₁₋₆ alkyl, C₁₋₆ alkoxy, haloC₁₋₆ alkoxy, C₂₋₆ alkenyl, C₂₋₆ alkynyl, 3-7 membered heterocyclyl, 3-7 membered cycloalkyl, aryl, and 5-7 membered heteroaryl;
R₂ is selected from hydrogen, hydroxyl, amino, carboxyl, cyano, nitro, halogen, C₁₋₆ alkyl, haloC₁₋₆ alkyl, C₁₋₆ alkoxy, haloC₁₋₆ alkoxy, C₂₋₆ alkenyl, C₂₋₆ alkynyl, 3-7 membered heterocyclyl, 3-7 membered cycloalkyl, aryl, and 5-7 membered heteroaryl;
R₁ and R₂ are respectively optionally substituted with 1-3 substituents selected from hydroxyl, amino, carboxyl, cyano, nitro, halogen, carbonyl, C₁₋₆ alkyl, haloC₁₋₆ alkyl, C₁₋₆ alkoxy, haloC₁₋₆ alkoxy, 3-7 membered heterocyclyl, 3-7 membered cycloalkyl, aryl, and 5-7 membered heteroaryl;
or,
R₁ and R₂, together with the carbon atom to which they are attached, form a 5-12 membered ring A, wherein the 5-12 membered ring A is optionally substituted with 1-4 substituents selected from hydroxyl, amino, carboxyl, cyano, nitro, halogen, carbonyl, C₁₋₆ alkyl, —NH—C₁₋₆ alkyl, haloC₁₋₆ alkyl, C₁₋₆ alkoxy, haloC₁₋₆ alkoxy, 3-7 membered heterocyclyl, 3-7 membered cycloalkyl, aryl, 5-7 membered heteroaryl, C₁₋₆ alkylsulfonyl, and —N(C₁₋₆ alkyl)₂;
R₃ is selected from —(C₁₋₆ alkylene)₀₋₂-NR₄R₅, —(C₁₋₆ alkylene)₀₋₂-NR₄—COR₅, —(C₁₋₆ alkylene)₀₋₂-CO—NR₄-R₅, and —(C₁₋₆ alkylene)₀₋₂-O—R₅; R₄ is selected from hydrogen and C₁₋₆ alkyl; R₅ is selected from 3-7 membered heterocyclyl, 3-7 membered cycloalkyl, aryl, and 5-7 membered heteroaryl; R₅ is optionally substituted with 1-4 substituents selected from halogen, cyano, amino, hydroxyl, C₁₋₆ alkyl, haloC₁₋₆ alkyl, C₁₋₆ alkoxy, 3-7 membered heterocyclyl, 3-7 membered cycloalkyl, aryl, 5-7 membered heteroaryl, C₂₋₆ alkenylcarbonyl, sulfonyl, C₁₋₆ alkylcarbonyl, and carboxyl;
Y is selected from aryl, 5-14 membered heteroaryl, 3-14 membered heterocyclyl, and 3-12 membered cycloalkyl, and Y is optionally substituted with 1-3 substituents selected from halogen, cyano, amino, hydroxyl, carbonyl, C₁₋₆ alkyl, haloC₁₋₆ alkyl, C₁₋₆ alkoxy, 3-7 membered heterocyclyl, 3-7 membered cycloalkyl, aryl, 5-7 membered heteroaryl, C₁₋₆ alkylamino, C₁₋₆ alkylcarbonylamino, C₁₋₆ alkylsulfonyl, aminocarbonyl, C₁₋₆ alkylaminocarbonyl, sulfonyl, C₁₋₆ alkylthio, and C₁₋₆ alkylsulfinyl;
when R₅ is substituted,
the substituents on R₅, which are C₁₋₆ alkyl, haloC₁₋₆ alkyl, C₁₋₆ alkoxy, 3-7 membered heterocyclyl, 3-7 membered cycloalkyl, aryl, 5-7 membered heteroaryl, and sulfonyl, are optionally substituted with 1-3 substituents selected from halogen, cyano, amino, hydroxyl, carbonyl, C₁₋₆ alkyl, and 3-6 membered cycloalkyl;
when Y is substituted,
the substituents on Y, which are C₁₋₆ alkyl, haloC₁₋₆ alkyl, C₁₋₆ alkoxy, 3-7 membered heterocyclyl, 3-7 membered cycloalkyl, aryl, 5-7 membered heteroaryl, and sulfonyl, are optionally substituted with 1-3 substituents selected from halogen, cyano, amino, hydroxyl, carbonyl, C₁₋₆ alkyl, and 3-6 membered cycloalkyl.

On the basis of the above technical solutions, the compound, or the pharmaceutically acceptable salt, the stereoisomer or the tautomer thereof provided in the present invention has a structure of general formula (II):

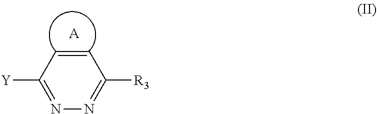

wherein ring A is selected from 5-7 membered cycloalkenyl, 5-7 membered cycloalkyl, 5-7 membered heterocyclyl, phenyl, and 5-7 membered heteroaryl; ring A is optionally substituted with 1-4 substituents selected from hydroxyl, amino, carboxyl, cyano, nitro, halogen, C₁₋₆ alkyl, —NH—C₁₋₆ alkyl, haloC₁₋₆ alkyl, C₁₋₆ alkoxy, haloC₁₋₆ alkoxy, 3-7 membered heterocyclyl, 3-7 membered cycloalkyl, aryl, 5-7 membered heteroaryl, C₁₋₆ alkylsulfonyl, and —N(C₁₋₆ alkyl)₂.

On the basis of the above technical solutions, for the compound, or the pharmaceutically acceptable salt, the stereoisomer or the tautomer thereof provided in the present invention, wherein ring A is selected from phenyl and 5-7 membered heteroaryl; ring A is optionally substituted with 1-4 substituents selected from hydroxyl, amino, carboxyl, cyano, nitro, halogen, C₁₋₆ alkyl, —NH—C₁₋₆ alkyl, haloC₁₋₆ alkyl, C₁₋₆ alkoxy, haloC₁₋₆ alkoxy, 3-7 membered heterocyclyl, 3-7 membered cycloalkyl, C₁₋₆ alkylsulfonyl, and —N(C₁₋₆ alkyl)₂.

On the basis of the above technical solutions, for the compound, or the pharmaceutically acceptable salt, the stereoisomer or the tautomer thereof provided in the present invention, wherein ring A is selected from,

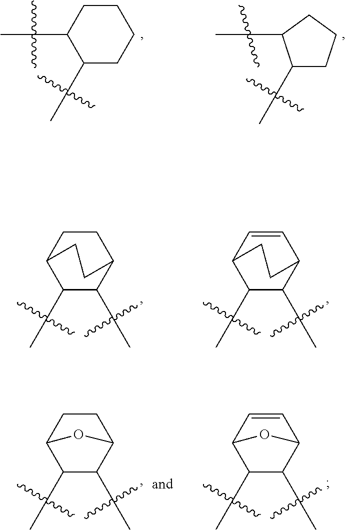

ring A is optionally substituted with 1-4 substituents selected from hydroxyl, amino, carboxyl, cyano, nitro, halogen, C₁₋₆ alkyl, —NH—C₁₋₆ alkyl, haloC₁₋₆ alkyl, C₁₋₆ alkoxy, haloC₁₋₆ alkoxy, 3-7 membered heterocyclyl, 3-7 membered cycloalkyl, C₁₋₆ alkylsulfonyl, and —N(C₁₋₆ alkyl)₂.

On the basis of the above technical solutions, for the compound, or the pharmaceutically acceptable salt, the stereoisomer or the tautomer thereof provided in the present invention, wherein Y is selected from phenyl, 5-7 membered heteroaryl, 3-8 membered heterocyclyl, and 3-7 membered cycloalkyl; Y is optionally substituted with 1-3 substituents selected from halogen, cyano, amino, hydroxyl, carbonyl, C₁₋₆ alkyl, haloC₁₋₆ alkyl, C₁₋₆ alkoxy, 3-7 membered heterocyclyl, 3-7 membered cycloalkyl, C₁₋₆ alkylamino, C₁₋₆ alkylcarbonylamino, C₁₋₆ alkylsulfonyl, aminocarbonyl, C₁₋₆ alkylaminocarbonyl, and sulfonyl; the C₁₋₆ alkyl, haloC₁₋₆ alkyl, C₁₋₆ alkoxy, 3-7-membered heterocyclyl, 3-7-membered cycloalkyl and sulfonyl are optionally substituted by 1-3 substituents selected from halogen, cyano, amino, hydroxyl, carbonyl, and C₁₋₆ alkyl.

On the basis of the above technical solutions, for the compound, or the pharmaceutically acceptable salt, the stereoisomer or the tautomer thereof provided in the present invention, wherein Y is selected from naphthyl, 8-14 membered fused heteroaryl, 6-12 membered fused heterocyclyl, and 6-12 membered fused cycloalkyl; Y is optionally substituted with 1-3 substituents selected from halogen, cyano, amino, hydroxyl, carbonyl, C₁₋₆ alkyl, haloC₁₋₆ alkyl, and C₁₋₆ alkoxy.

On the basis of the above technical solutions, for the compound, or the pharmaceutically acceptable salt, the stereoisomer or the tautomer thereof provided in the present invention, wherein Y is substituted with cyano and is optionally substituted with 1-2 substituents selected from halogen, cyano, amino, hydroxyl, carbonyl, C₁₋₆ alkyl, haloC₁₋₆ alkyl, C₁₋₆ alkoxy, 3-7-membered heterocyclyl, 3-7-membered cycloalkyl, C₁₋₆ alkylamino, C₁₋₆ alkylcarbonylamino, C₁₋₆ alkylsulfonyl, aminocarbonyl, C₁₋₆ alkylaminocarbonyl, and sulfonyl; the C₁₋₆ alkyl, haloC₁₋₆ alkyl, C₁₋₆ alkoxy, 3-7-membered heterocyclyl, 3-7-membered cycloalkyl and sulfonyl are optionally substituted with 1-3 substituents selected from halogen, cyano, amino, hydroxyl, carbonyl, and C₁₋₆ alkyl.

On the basis of the above technical solutions, for the compound, or the pharmaceutically acceptable salt, the stereoisomer or the tautomer thereof provided in the present invention, wherein R₃ is selected from —NR₄R₅, —NH—COR₅, and —O—R₅; R₄ is selected from hydrogen and C₁₋₃ alkyl; R₅ is selected from 3-7 membered cycloalkyl and 3-7 membered heterocyclyl, and R₅ is optionally substituted with 1-2 substituents selected from C₁₋₆ alkyl, hydroxyl, hydroxyl-substituted C₁₋₆ alkyl, 3-7 membered cycloalkyl-substituted C₁₋₆ alkyl, hydroxyl, halogen, C₂₋₆ alkenylcarbonyl, C₁₋₆ alkylsulfonyl, 3-7 membered cycloalkylsulfonyl, aminosulfonyl, 3-7 membered heterocyclyl, 3-7 membered cycloalkyl, carboxyl, and C₁₋₆ alkylcarbonyl.

On the basis of the above technical solutions, for the compound, or the pharmaceutically acceptable salt, the stereoisomer or the tautomer thereof provided in the present invention, wherein R₃ is —NH—R₅, and R₅ is 3-7 membered heterocyclyl substituted with 1-2 substituents selected from C₁₋₆ alkyl.

On the basis of the above technical solutions, for the compound, or the pharmaceutically acceptable salt, the stereoisomer or the tautomer thereof provided in the present invention, wherein Y is selected from phenyl, 5-7 membered heteroaryl, 3-8 membered heterocyclyl, and 3-7 membered cycloalkyl; Y is optionally substituted with 1-3 substituents selected from halogen, cyano, amino, hydroxyl, carbonyl, C₁₋₆ alkyl, haloC₁₋₆ alkyl, C₁₋₆ alkoxy, 3-7 membered heterocyclyl, 3-7 membered cycloalkyl, C₁₋₆ alkylamino, C₁₋₆ alkylcarbonylamino, C₁₋₆ alkylsulfonyl, aminocarbonyl, C₁₋₆ alkylaminocarbonyl, and sulfonyl; the C₁₋₆ alkyl, haloC₁₋₆ alkyl, C₁₋₆ alkoxy, 3-7 membered heterocyclyl, 3-7 membered cycloalkyl and sulfonyl are optionally substituted with 1-3 substituents selected from halogen, cyano, amino, hydroxyl, carbonyl, and C₁₋₆ alkyl; R₃ is —NH—R₅, and R₅ is 3-7 membered heterocyclyl substituted with 1-2 substituents selected from C₁₋₆ alkyl.

On the basis of the above technical solutions, for the compound, or the pharmaceutically acceptable salt, the stereoisomer or the tautomer thereof provided in the present invention, wherein R₁ and R₂, together with the carbon atom to which they are attached, form a 5-8 membered ring A, wherein the 5-8 membered ring A is selected from 5-8 membered cycloalkyl, 5-8 membered cycloalkenyl, 5-8 membered heterocyclyl, phenyl, and 5-8 membered heteroaryl; ring A is optionally substituted with 1-2 substituents selected from cyano, halogen, C₁₋₆ alkyl, —NH—C₁₋₆ alkyl, haloC₁₋₆ alkyl, C₁₋₆ alkoxy, 3-7 membered cycloalkyl, 5-7 membered heteroaryl, C₁₋₆ alkylsulfonyl, and —N(C₁₋₆ alkyl)₂;
Y is selected from phenyl and 5-7 membered heteroaryl; Y is substituted with 1-3 substituents selected from halogen, cyano, hydroxyl, amino, C₁₋₆ alkyl, C₁₋₆ alkoxy, hydroxyl-substituted C₁₋₆ alkyl, haloC₁₋₆ alkyl, aminocarbonyl, C₁₋₆ alkylamino, C₁₋₆ alkylcarbonylamino, C₁₋₆ alkylaminocarbonyl, 3-7 membered heterocyclyl, 3-7 membered cycloalkyl, 5-7 membered heteroaryl, C₁₋₆ alkylsulfonyl, C₁₋₆ alkylthio, and C₁₋₆ alkylsulfinyl;
R₃ is selected from —NR₄R₅, —NH—COR₅, and —O—R₅; R₄ is selected from hydrogen and C₁₋₃ alkyl; R₅ is selected from 3-7 membered cycloalkyl and 3-7 membered heterocyclyl, and R₅ is optionally substituted with 1-2 substituents selected from C₁₋₆ alkyl, hydroxyl or 3-7 membered cycloalkyl-substituted C₁₋₆ alkyl, hydroxyl, halogen, C₂₋₆ alkenylcarbonyl, C₁₋₆ alkylsulfonyl, 3-7 membered cycloalkylsulfonyl, aminosulfonyl, 3-7 membered heterocyclyl, 3-7 membered cycloalkyl, carboxyl, and C₁₋₆ alkylcarbonyl.

On the basis of the above technical solutions, for the compound, or the pharmaceutically acceptable salt, the stereoisomer or the tautomer thereof provided in the

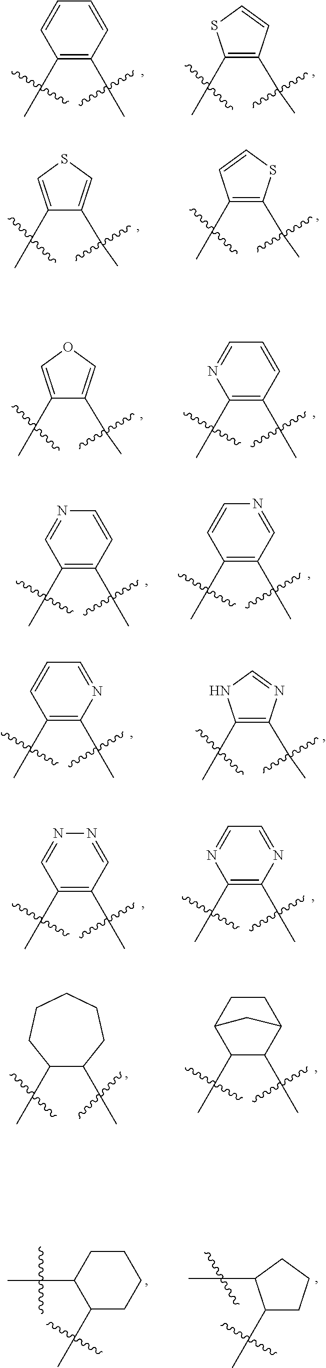

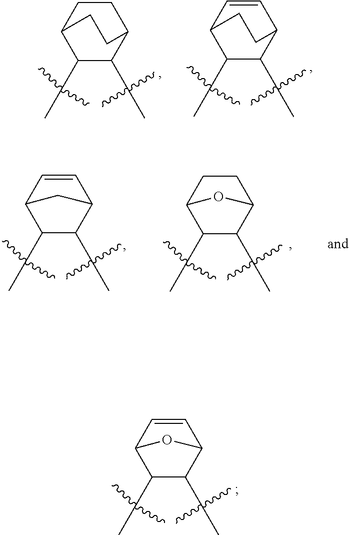 [SMILES: *C1C2CCC(CC2)C1* ; *C1C2C=CC(CC2)C1* ; *C1C2C=CC(C2)C1* ; *C1C2CCC(O2)C1* ; *C1C2C=CC(O2)C1*]

ring A is optionally substituted with 1-2 substituents selected from cyano, halogen, C₁₋₆ alkyl, —NH—C₁₋₆ alkyl, haloC₁₋₆ alkyl, C₁₋₆ alkoxy, 3-7 membered cycloalkyl, 5-7 membered heteroaryl, C₁₋₆ alkylsulfonyl, and —N(C₁₋₆ alkyl)₂.

On the basis of the above technical solutions, for the compound, or the pharmaceutically acceptable salt, the stereoisomer or the tautomer thereof provided in the present invention, wherein Y is selected from

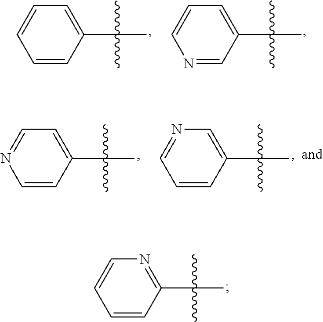

Y is substituted with 1-3 substituents selected from halogen, cyano, hydroxyl, amino, C₁₋₆ alkyl, C₁₋₆ alkoxy, hydroxyl-substituted C₁₋₆ alkyl, haloC₁₋₆ alkyl, aminocarbonyl, C₁₋₆ alkylamino, C₁₋₆ alkylcarbonylamino, C₁₋₆ alkylaminocarbonyl, 3-7 membered heterocyclyl, 3-7 membered cycloalkyl, 5-7 membered heteroaryl, C₁₋₆ alkylsulfonyl, C₁₋₆ alkylthio, and C₁₋₆ alkylsulfinyl.

The present invention also provides the aforementioned compound of formula (I) or (II), the pharmaceutically acceptable salt thereof, or the stereoisomer or the tautomer thereof as shown in Table 1:

TABLE 1

| No. | Structure |
|---|---|
| 1 | 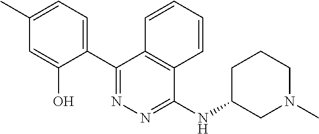 |
| 2 | 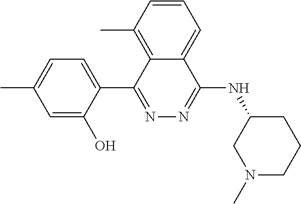 [SMILES: Cc1ccc(-c2nnc(N[C@@H]3CCCN(C)C3)c3cccc(C)c23)c(O)c1] |
| 3 | 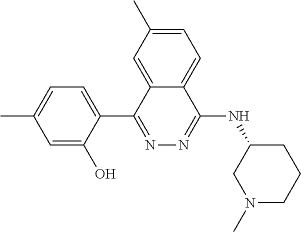 [SMILES: Cc1ccc(-c2nnc(N[C@@H]3CCCN(C)C3)c3ccc(C)cc23)c(O)c1] |
| 4 | 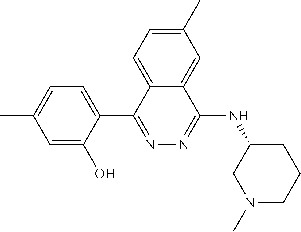 [SMILES: Cc1ccc(-c2nnc(N[C@@H]3CCCN(C)C3)c3cc(C)ccc23)c(O)c1] |
| 5 | 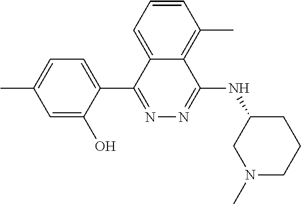 [SMILES: Cc1ccc(-c2nnc(N[C@@H]3CCCN(C)C3)c3c(C)cccc23)c(O)c1] |
| 6 | 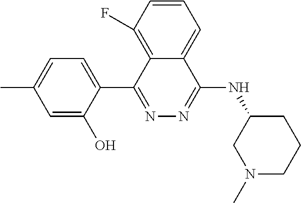 [SMILES: Cc1ccc(-c2nnc(N[C@@H]3CCCN(C)C3)c3cccc(F)c23)c(O)c1] |
| 7 | 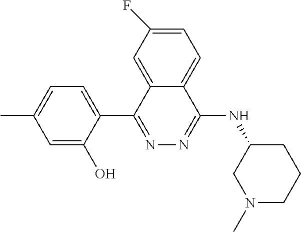 [SMILES: Cc1ccc(-c2nnc(N[C@@H]3CCCN(C)C3)c3ccc(F)cc23)c(O)c1] |
| 8 | 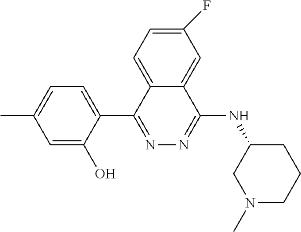 [SMILES: Cc1ccc(-c2nnc(N[C@@H]3CCCN(C)C3)c3cc(F)ccc23)c(O)c1] |
| 9 | 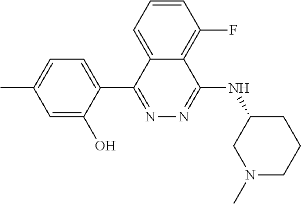 [SMILES: Cc1ccc(-c2nnc(N[C@@H]3CCCN(C)C3)c3c(F)cccc23)c(O)c1] |
| 10 | 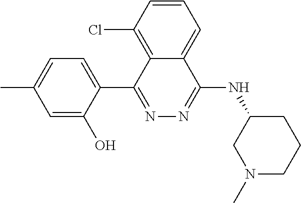 [SMILES: Cc1ccc(-c2nnc(N[C@@H]3CCCN(C)C3)c3cccc(Cl)c23)c(O)c1] |
| 11 | 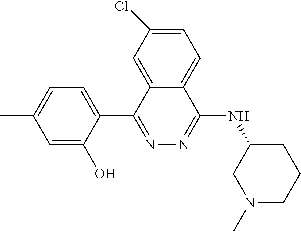 [SMILES: Cc1ccc(-c2nnc(N[C@@H]3CCCN(C)C3)c3ccc(Cl)cc23)c(O)c1] |
| 12 | 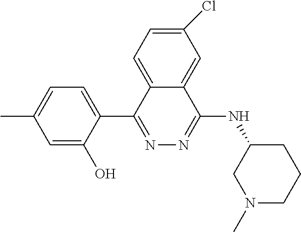 [SMILES: Cc1ccc(-c2nnc(N[C@@H]3CCCN(C)C3)c3cc(Cl)ccc23)c(O)c1] |
| 13 | 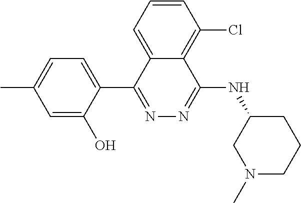 [SMILES: Cc1ccc(-c2nnc(N[C@@H]3CCCN(C)C3)c3c(Cl)cccc23)c(O)c1] |
| 14 | 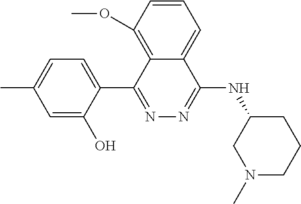 [SMILES: COc1cccc2c(N[C@@H]3CCCN(C)C3)nnc(-c3ccc(C)cc3O)c12] |
| 15 | 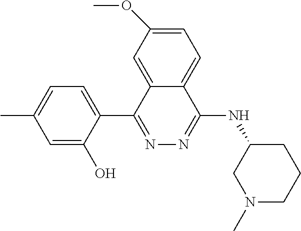 [SMILES: COc1ccc2c(N[C@@H]3CCCN(C)C3)nnc(-c3ccc(C)cc3O)c2c1] |
| 16 | 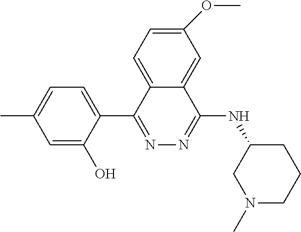 [SMILES: COc1ccc2c(-c3ccc(C)cc3O)nnc(N[C@@H]3CCCN(C)C3)c2c1] |
| 17 | 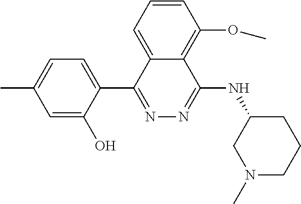 [SMILES: COc1cccc2c(-c3ccc(C)cc3O)nnc(N[C@@H]3CCCN(C)C3)c12] |
| 18 | 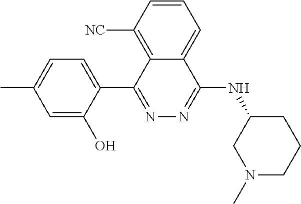 [SMILES: Cc1ccc(-c2nnc(N[C@@H]3CCCN(C)C3)c3cccc(C#N)c23)c(O)c1] |
| 19 | 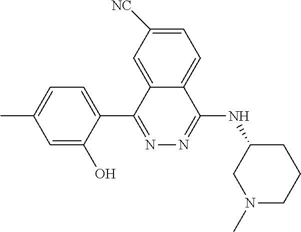 [SMILES: Cc1ccc(-c2nnc(N[C@@H]3CCCN(C)C3)c3ccc(C#N)cc23)c(O)c1] |
| 20 | 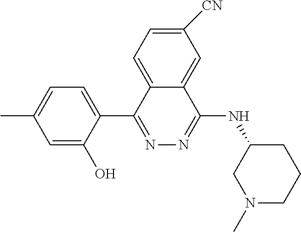 [SMILES: Cc1ccc(-c2nnc(N[C@@H]3CCCN(C)C3)c3cc(C#N)ccc23)c(O)c1] |
| 21 | 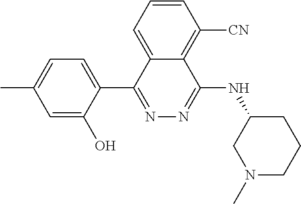 [SMILES: Cc1ccc(-c2nnc(N[C@@H]3CCCN(C)C3)c3c(C#N)cccc23)c(O)c1] |
| 22 | 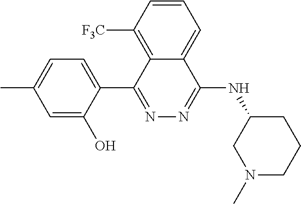 [SMILES: Cc1ccc(-c2nnc(N[C@@H]3CCCN(C)C3)c3cccc(C(F)(F)F)c23)c(O)c1] |
| 23 | 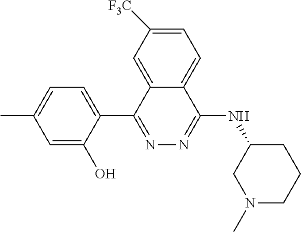 [SMILES: Cc1ccc(-c2nnc(N[C@@H]3CCCN(C)C3)c3ccc(C(F)(F)F)cc23)c(O)c1] |
| 24 | 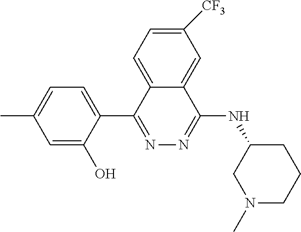 [SMILES: Cc1ccc(-c2nnc(N[C@@H]3CCCN(C)C3)c3cc(C(F)(F)F)ccc23)c(O)c1] |
| 25 | 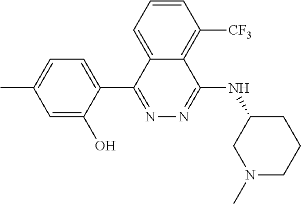 [SMILES: Cc1ccc(-c2nnc(N[C@@H]3CCCN(C)C3)c3c(C(F)(F)F)cccc23)c(O)c1] |
| 26 | 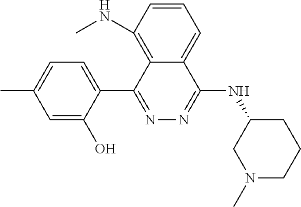 [SMILES: CNc1cccc2c(N[C@@H]3CCCN(C)C3)nnc(-c3ccc(C)cc3O)c12] |
| 27 | 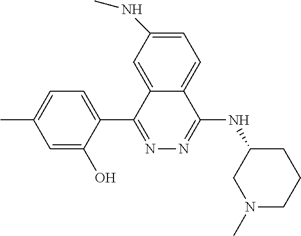 [SMILES: CNc1ccc2c(N[C@@H]3CCCN(C)C3)nnc(-c3ccc(C)cc3O)c2c1] |
| 28 | 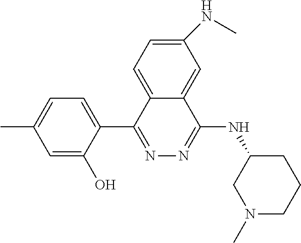 [SMILES: CNc1ccc2c(-c3ccc(C)cc3O)nnc(N[C@@H]3CCCN(C)C3)c2c1] |
| 29 | 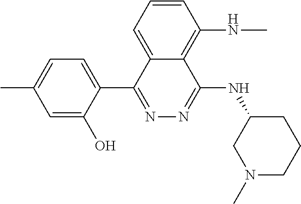 [SMILES: CNc1cccc2c(-c3ccc(C)cc3O)nnc(N[C@@H]3CCCN(C)C3)c12] |
| 30 | 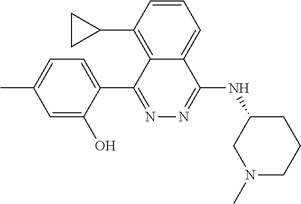 [SMILES: Cc1ccc(-c2nnc(N[C@@H]3CCCN(C)C3)c3cccc(C4CC4)c23)c(O)c1] |
| 31 | 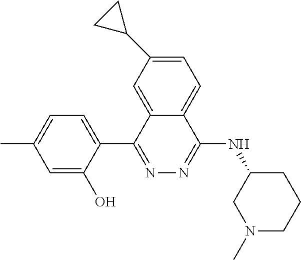 [SMILES: Cc1ccc(-c2nnc(N[C@@H]3CCCN(C)C3)c3ccc(C4CC4)cc23)c(O)c1] |
| 32 | 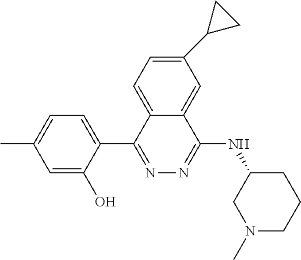 [SMILES: Cc1ccc(-c2nnc(N[C@@H]3CCCN(C)C3)c3cc(C4CC4)ccc23)c(O)c1] |
| 33 | 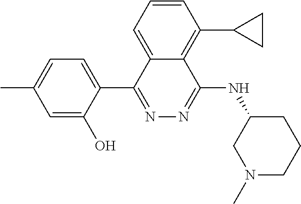 [SMILES: Cc1ccc(-c2nnc(N[C@@H]3CCCN(C)C3)c3c(C4CC4)cccc23)c(O)c1] |
| 34 | 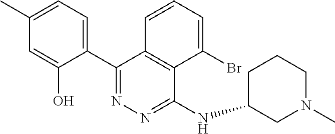 |
| 35 | 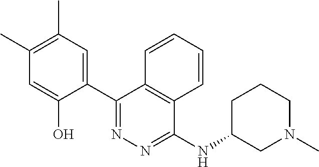 |
| 36 | 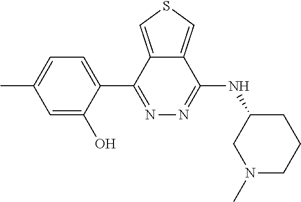 [SMILES: Cc1ccc(-c2nnc(N[C@@H]3CCCN(C)C3)c3cscc23)c(O)c1] |
| 37 | 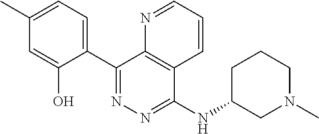 |
| 38 | 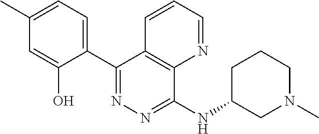 |
| 39 | 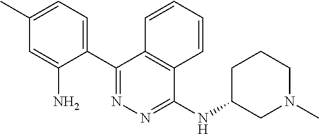 |
| 40 | 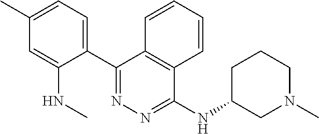 |
| 41 | 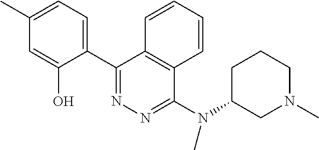 |
| 42 | 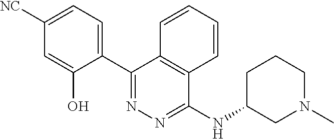 [SMILES: CN1CCC[C@@H](Nc2nnc(-c3ccc(C#N)cc3O)c3ccccc23)C1] |
| 43 | 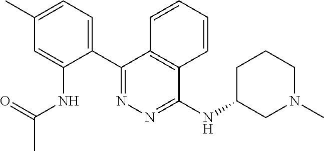 |
| 44 | 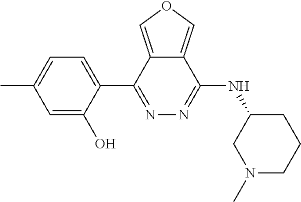 [SMILES: Cc1ccc(-c2nnc(N[C@@H]3CCCN(C)C3)c3cocc23)c(O)c1] |
| 45 | 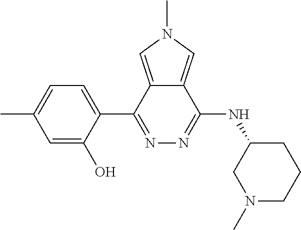 [SMILES: Cc1ccc(-c2nnc(N[C@@H]3CCCN(C)C3)c3cn(C)cc23)c(O)c1] |
| 46 | 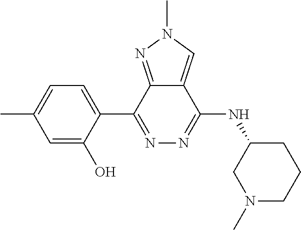 [SMILES: Cc1ccc(-c2nnc(N[C@@H]3CCCN(C)C3)c3cn(C)nc23)c(O)c1] |
| 47 | 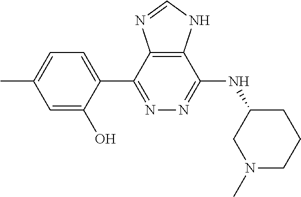 [SMILES: Cc1ccc(-c2nnc(N[C@@H]3CCCN(C)C3)c3[nH]cnc23)c(O)c1] |
| 48 | 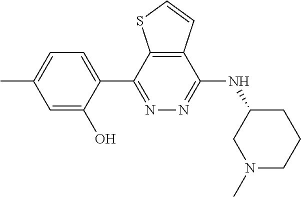 [SMILES: Cc1ccc(-c2nnc(N[C@@H]3CCCN(C)C3)c3ccsc23)c(O)c1] |
| 49 | 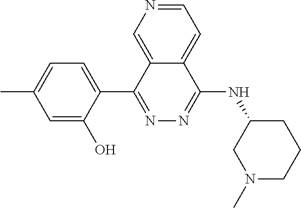 [SMILES: Cc1ccc(-c2nnc(N[C@@H]3CCCN(C)C3)c3ccncc23)c(O)c1] |
| 50 | 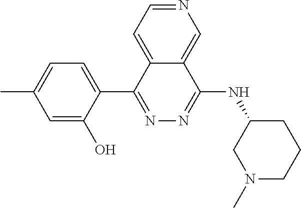 [SMILES: Cc1ccc(-c2nnc(N[C@@H]3CCCN(C)C3)c3cnccc23)c(O)c1] |
| 51 | 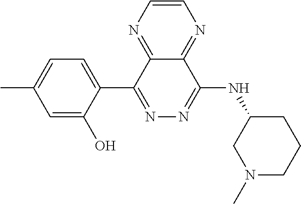 [SMILES: Cc1ccc(-c2nnc(N[C@@H]3CCCN(C)C3)c3nccnc23)c(O)c1] |
| 52 | 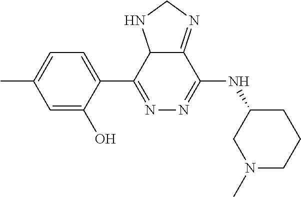 [SMILES: Cc1ccc(C2=NN=C(N[C@@H]3CCCN(C)C3)C3=NCNC32)c(O)c1] |
| 53 | 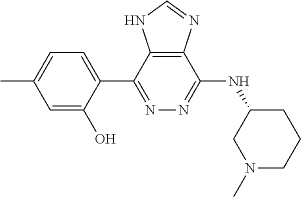 [SMILES: Cc1ccc(-c2nnc(N[C@@H]3CCCN(C)C3)c3nc[nH]c23)c(O)c1] |
| 54 | 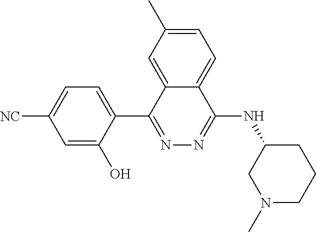 [SMILES: Cc1ccc2c(N[C@@H]3CCCN(C)C3)nnc(-c3ccc(C#N)cc3O)c2c1] |
| 55 | 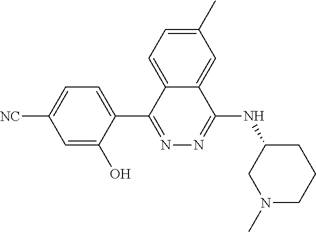 [SMILES: Cc1ccc2c(-c3ccc(C#N)cc3O)nnc(N[C@@H]3CCCN(C)C3)c2c1] |
| 56 | 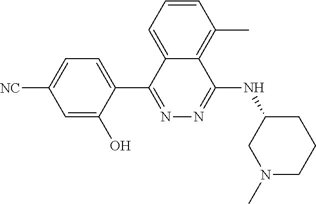 [SMILES: Cc1cccc2c(-c3ccc(C#N)cc3O)nnc(N[C@@H]3CCCN(C)C3)c12] |
| 57 | 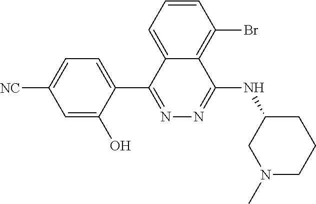 [SMILES: CN1CCC[C@@H](Nc2nnc(-c3ccc(C#N)cc3O)c3cccc(Br)c23)C1] |
| 58 | 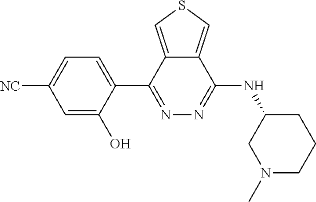 [SMILES: CN1CCC[C@@H](Nc2nnc(-c3ccc(C#N)cc3O)c3cscc23)C1] |
| 59 | 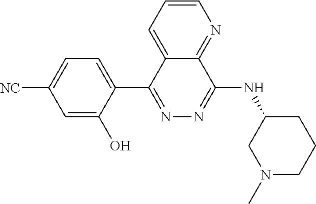 [SMILES: CN1CCC[C@@H](Nc2nnc(-c3ccc(C#N)cc3O)c3cccnc23)C1] |
| 60 | 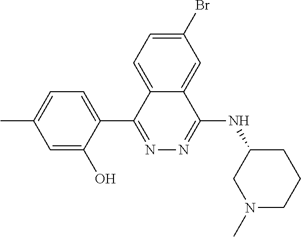 [SMILES: Cc1ccc(-c2nnc(N[C@@H]3CCCN(C)C3)c3cc(Br)ccc23)c(O)c1] |
| 61 | 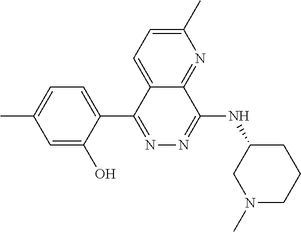 [SMILES: Cc1ccc(-c2nnc(N[C@@H]3CCCN(C)C3)c3nc(C)ccc23)c(O)c1] |
| 62 | 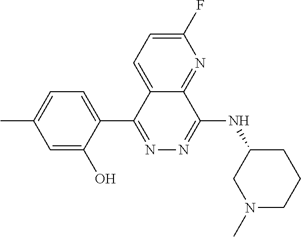 [SMILES: Cc1ccc(-c2nnc(N[C@@H]3CCCN(C)C3)c3nc(F)ccc23)c(O)c1] |
| 63 | 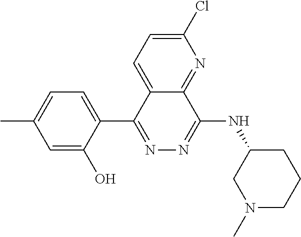 [SMILES: Cc1ccc(-c2nnc(N[C@@H]3CCCN(C)C3)c3nc(Cl)ccc23)c(O)c1] |
| 64 | 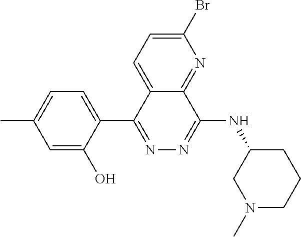 [SMILES: Cc1ccc(-c2nnc(N[C@@H]3CCCN(C)C3)c3nc(Br)ccc23)c(O)c1] |
| 65 | 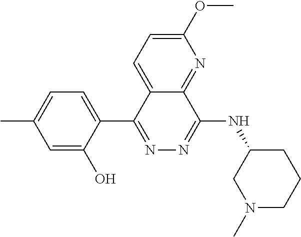 [SMILES: COc1ccc2c(-c3ccc(C)cc3O)nnc(N[C@@H]3CCCN(C)C3)c2n1] |
| 66 | 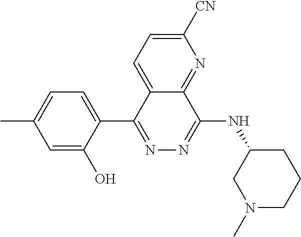 [SMILES: Cc1ccc(-c2nnc(N[C@@H]3CCCN(C)C3)c3nc(C#N)ccc23)c(O)c1] |
| 67 | 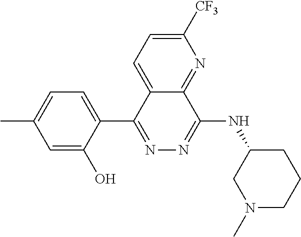 [SMILES: Cc1ccc(-c2nnc(N[C@@H]3CCCN(C)C3)c3nc(C(F)(F)F)ccc23)c(O)c1] |
| 68 | 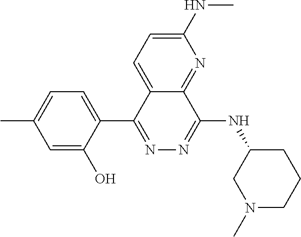 [SMILES: CNc1ccc2c(-c3ccc(C)cc3O)nnc(N[C@@H]3CCCN(C)C3)c2n1] |
| 69 | 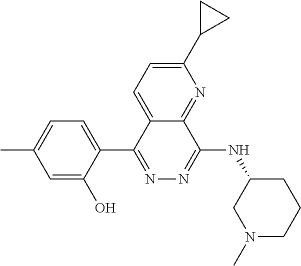 [SMILES: Cc1ccc(-c2nnc(N[C@@H]3CCCN(C)C3)c3nc(C4CC4)ccc23)c(O)c1] |
| 70 | 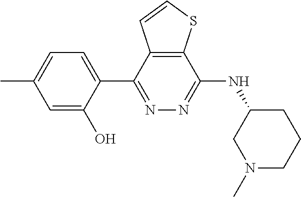 [SMILES: Cc1ccc(-c2nnc(N[C@@H]3CCCN(C)C3)c3sccc23)c(O)c1] |
| 71 | 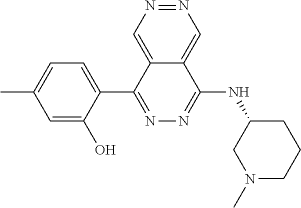 [SMILES: Cc1ccc(-c2nnc(N[C@@H]3CCCN(C)C3)c3cnncc23)c(O)c1] |
| 72 | 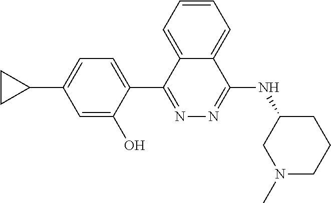 [SMILES: CN1CCC[C@@H](Nc2nnc(-c3ccc(C4CC4)cc3O)c3ccccc23)C1] |
| 73 | 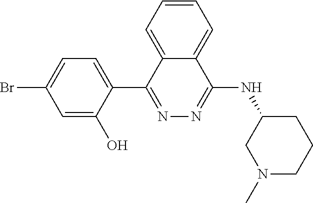 [SMILES: CN1CCC[C@@H](Nc2nnc(-c3ccc(Br)cc3O)c3ccccc23)C1] |
| 74 | 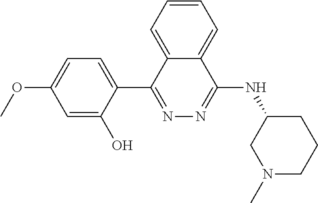 [SMILES: COc1ccc(-c2nnc(N[C@@H]3CCCN(C)C3)c3ccccc23)c(O)c1] |
| 75 | 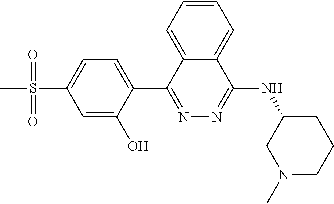 [SMILES: CN1CCC[C@@H](Nc2nnc(-c3ccc(S(C)(=O)=O)cc3O)c3ccccc23)C1] |
| 76 | 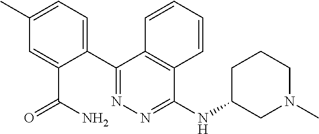 |
| 77 | 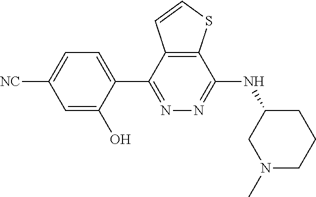 [SMILES: CN1CCC[C@@H](Nc2nnc(-c3ccc(C#N)cc3O)c3ccsc23)C1] |
| 78 | 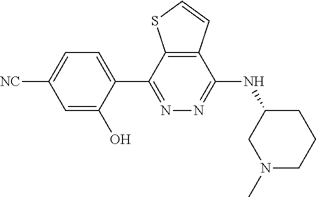 [SMILES: CN1CCC[C@@H](Nc2nnc(-c3ccc(C#N)cc3O)c3sccc23)C1] |
| 79 | 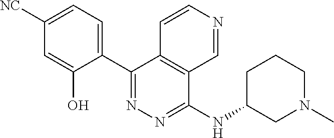 [SMILES: CN1CCC[C@@H](Nc2nnc(-c3ccc(C#N)cc3O)c3ccncc23)C1] |
| 80 | 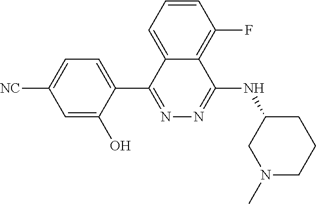 [SMILES: CN1CCC[C@@H](Nc2nnc(-c3ccc(C#N)cc3O)c3cccc(F)c23)C1] |
| 81 | 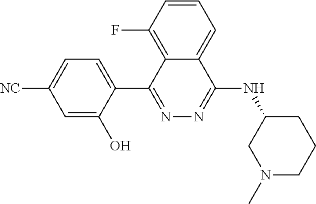 [SMILES: CN1CCC[C@@H](Nc2nnc(-c3ccc(C#N)cc3O)c3c(F)cccc23)C1] |
| 82 | 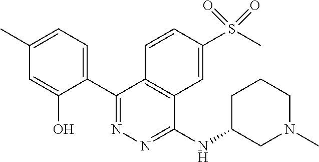 |
| 83 | 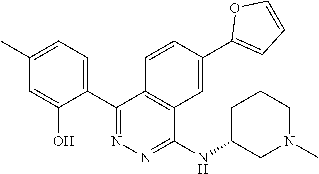 |
| 84 | 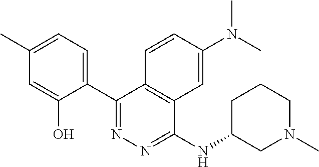 |
| 85 | 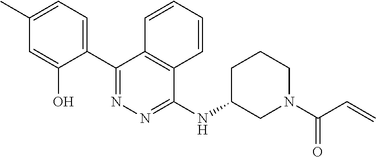 [SMILES: C=CC(=O)N1CCC[C@@H](Nc2nnc(-c3ccc(C)cc3O)c3ccccc23)C1] |
| 86 | 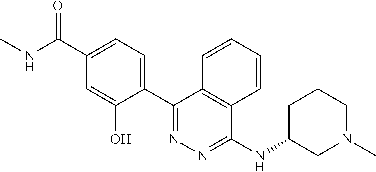 |
| 87 | 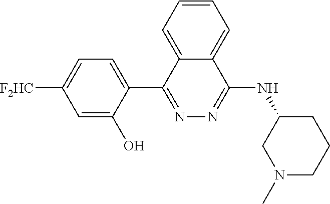 [SMILES: CN1CCC[C@@H](Nc2nnc(-c3ccc(C(F)F)cc3O)c3ccccc23)C1] |
| 88 | 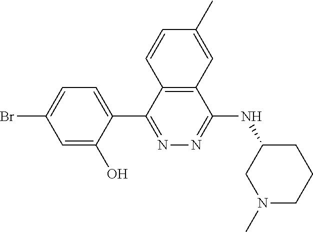 [SMILES: Cc1ccc2c(-c3ccc(Br)cc3O)nnc(N[C@@H]3CCCN(C)C3)c2c1] |
| 89 | 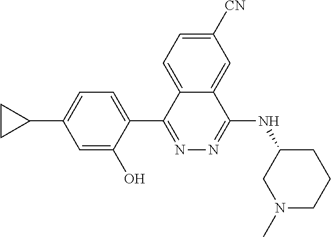 [SMILES: CN1CCC[C@@H](Nc2nnc(-c3ccc(C4CC4)cc3O)c3ccc(C#N)cc23)C1] |
| 90 | 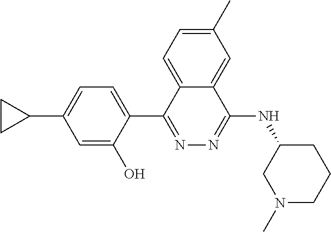 [SMILES: Cc1ccc2c(-c3ccc(C4CC4)cc3O)nnc(N[C@@H]3CCCN(C)C3)c2c1] |
| 91 | 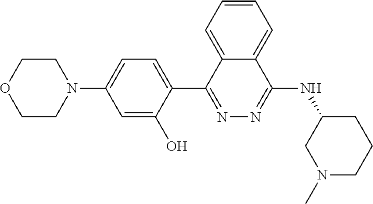 [SMILES: CN1CCC[C@@H](Nc2nnc(-c3ccc(N4CCOCC4)cc3O)c3ccccc23)C1] |
| 92 | 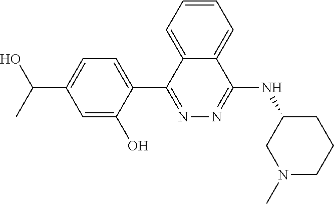 [SMILES: CC(O)c1ccc(-c2nnc(N[C@@H]3CCCN(C)C3)c3ccccc23)c(O)c1] |
| 93 | 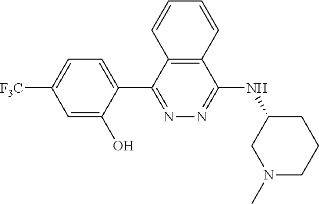 [SMILES: CN1CCC[C@@H](Nc2nnc(-c3ccc(C(F)(F)F)cc3O)c3ccccc23)C1] |
| 94 | 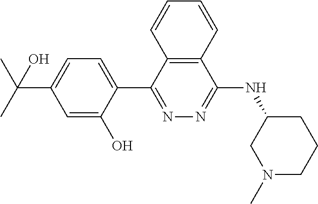 [SMILES: CN1CCC[C@@H](Nc2nnc(-c3ccc(C(C)(C)O)cc3O)c3ccccc23)C1] |
| 95 | 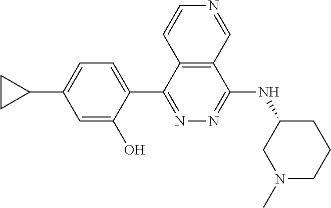 [SMILES: CN1CCC[C@@H](Nc2nnc(-c3ccc(C4CC4)cc3O)c3ccncc23)C1] |
| 96 | 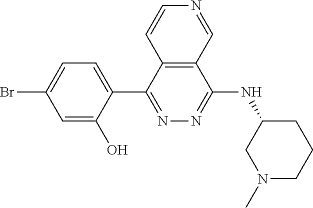 [SMILES: CN1CCC[C@@H](Nc2nnc(-c3ccc(Br)cc3O)c3ccncc23)C1] |
| 97 | 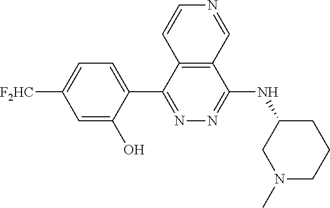 [SMILES: CN1CCC[C@@H](Nc2nnc(-c3ccc(C(F)F)cc3O)c3ccncc23)C1] |
| 101 | 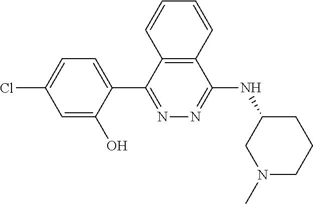 [SMILES: CN1CCC[C@@H](Nc2nnc(-c3ccc(Cl)cc3O)c3ccccc23)C1] |
| 102 | 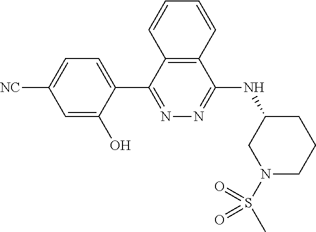 [SMILES: CS(=O)(=O)N1CCC[C@@H](Nc2nnc(-c3ccc(C#N)cc3O)c3ccccc23)C1] |
| 103 | 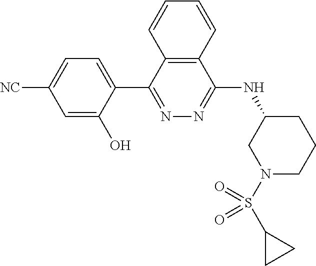 [SMILES: N#Cc1ccc(-c2nnc(N[C@@H]3CCCN(S(=O)(=O)C4CC4)C3)c3ccccc23)c(O)c1] |
| 104 | 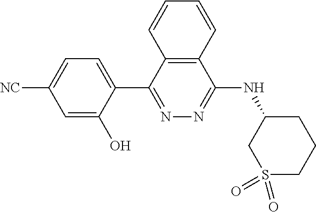 [SMILES: N#Cc1ccc(-c2nnc(N[C@@H]3CCCS(=O)(=O)C3)c3ccccc23)c(O)c1] |
| 105 | 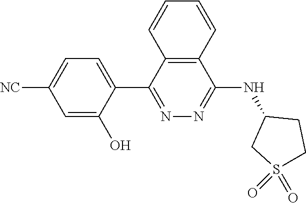 [SMILES: N#Cc1ccc(-c2nnc(N[C@@H]3CCS(=O)(=O)C3)c3ccccc23)c(O)c1] |
| 106 | 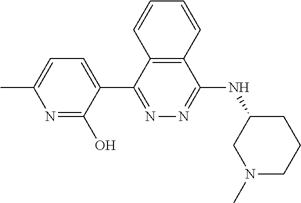 [SMILES: Cc1ccc(-c2nnc(N[C@@H]3CCCN(C)C3)c3ccccc23)c(O)n1] |
| 107 | 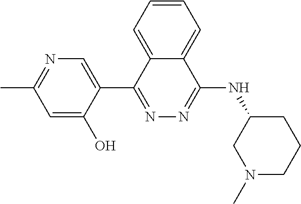 [SMILES: Cc1cc(O)c(-c2nnc(N[C@@H]3CCCN(C)C3)c3ccccc23)cn1] |
| 108 | 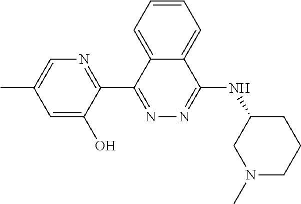 [SMILES: Cc1cnc(-c2nnc(N[C@@H]3CCCN(C)C3)c3ccccc23)c(O)c1] |
| 109 | 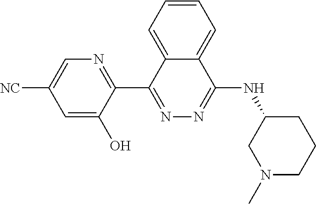 [SMILES: CN1CCC[C@@H](Nc2nnc(-c3ncc(C#N)cc3O)c3ccccc23)C1] |
| 110 | 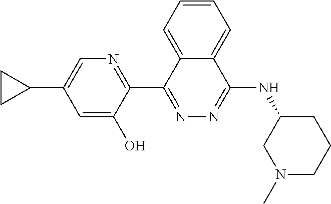 [SMILES: CN1CCC[C@@H](Nc2nnc(-c3ncc(C4CC4)cc3O)c3ccccc23)C1] |
| 111 | 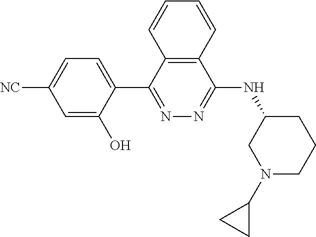 [SMILES: N#Cc1ccc(-c2nnc(N[C@@H]3CCCN(C4CC4)C3)c3ccccc23)c(O)c1] |
| 112 | 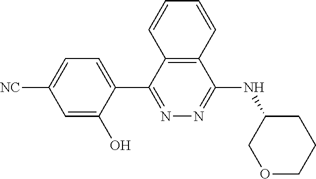 [SMILES: N#Cc1ccc(-c2nnc(N[C@@H]3CCCOC3)c3ccccc23)c(O)c1] |
| 113 | 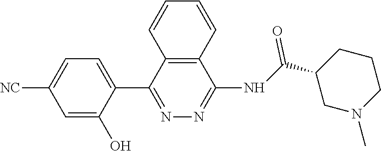 [SMILES: CN1CCC[C@@H](C(=O)Nc2nnc(-c3ccc(C#N)cc3O)c3ccccc23)C1] |
| 114 | 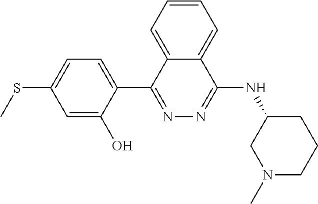 [SMILES: CSc1ccc(-c2nnc(N[C@@H]3CCCN(C)C3)c3ccccc23)c(O)c1] |
| 115 | 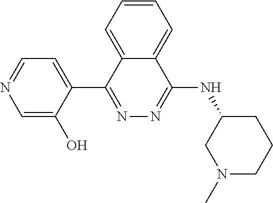 [SMILES: CN1CCC[C@@H](Nc2nnc(-c3ccncc3O)c3ccccc23)C1] |
| 116 | 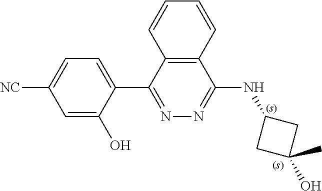 |
| 117 | 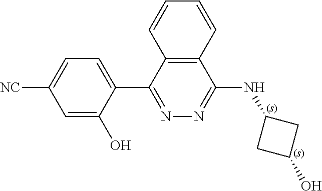 |
| 118 | 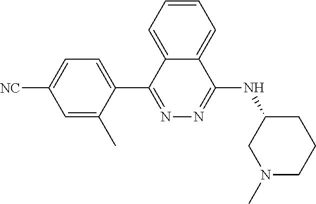 [SMILES: Cc1cc(C#N)ccc1-c1nnc(N[C@@H]2CCCN(C)C2)c2ccccc12] |
| 119 | 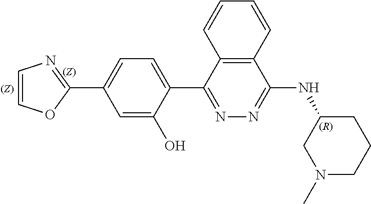 |
| 120 | 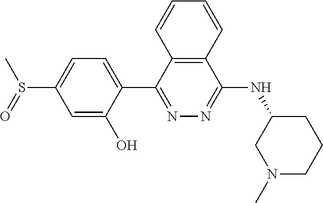 [SMILES: CN1CCC[C@@H](Nc2nnc(-c3ccc(S(C)=O)cc3O)c3ccccc23)C1] |
| 121 | 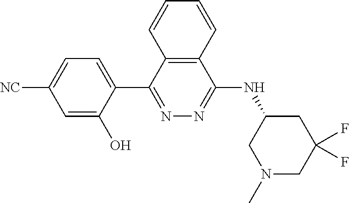 [SMILES: CN1C[C@H](Nc2nnc(-c3ccc(C#N)cc3O)c3ccccc23)CC(F)(F)C1] |
| 122 | 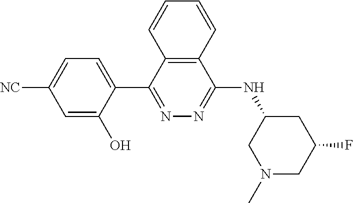 [SMILES: CN1C[C@@H](F)C[C@@H](Nc2nnc(-c3ccc(C#N)cc3O)c3ccccc23)C1] |
| 123 | 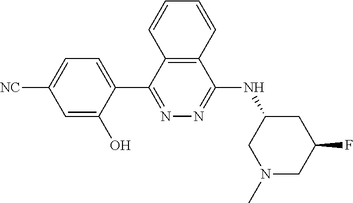 [SMILES: CN1C[C@H](F)C[C@@H](Nc2nnc(-c3ccc(C#N)cc3O)c3ccccc23)C1] |
| 124 | 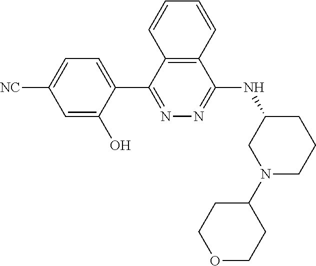 [SMILES: N#Cc1ccc(-c2nnc(N[C@@H]3CCCN(C4CCOCC4)C3)c3ccccc23)c(O)c1] |
| 125 | 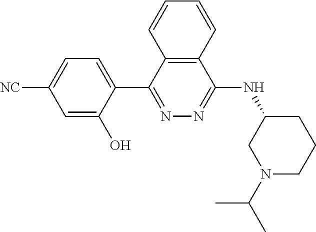 [SMILES: CC(C)N1CCC[C@@H](Nc2nnc(-c3ccc(C#N)cc3O)c3ccccc23)C1] |
| 126 | 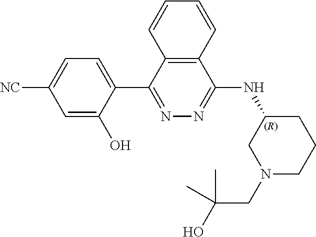 |
| 127 | 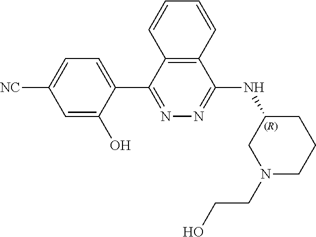 |
| 128 | 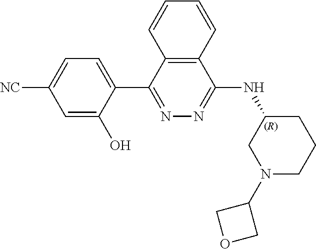 |
| 129 | 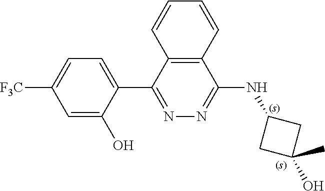 |
| 130 | 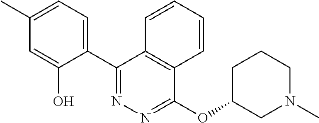 |
| 131 | 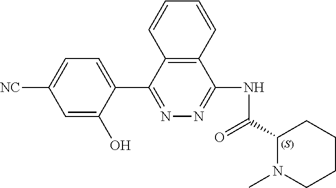 |
| 132 | 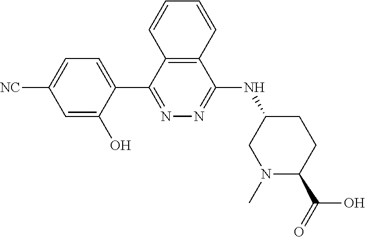 [SMILES: CN1C[C@H](Nc2nnc(-c3ccc(C#N)cc3O)c3ccccc23)CC[C@H]1C(=O)O] |
| 133 | 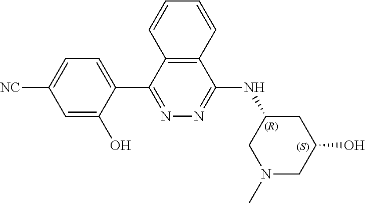 |
| 134 | 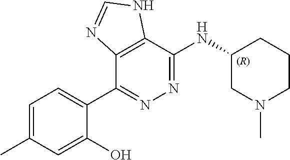 |
| 1a | 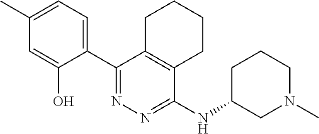 |
| 2a | 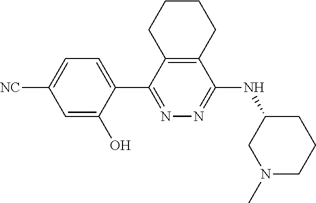 [SMILES: CN1CCC[C@@H](Nc2nnc(-c3ccc(C#N)cc3O)c3c2CCCC3)C1] |
| 3a | 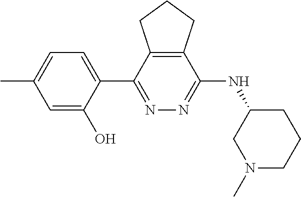 [SMILES: Cc1ccc(-c2nnc(N[C@@H]3CCCN(C)C3)c3c2CCC3)c(O)c1] |
| 4a | 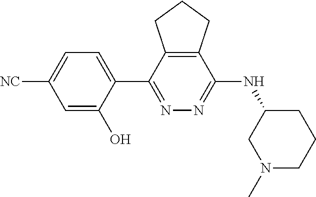 [SMILES: CN1CCC[C@@H](Nc2nnc(-c3ccc(C#N)cc3O)c3c2CCC3)C1] |
| 5a | 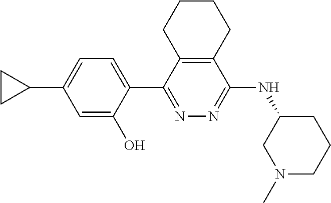 [SMILES: CN1CCC[C@@H](Nc2nnc(-c3ccc(C4CC4)cc3O)c3c2CCCC3)C1] |
| 6a | 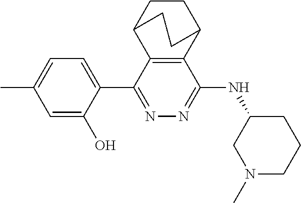 [SMILES: Cc1ccc(-c2nnc(N[C@@H]3CCCN(C)C3)c3c2C2CCC3CC2)c(O)c1] |
| 7a | 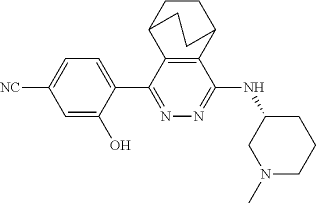 [SMILES: CN1CCC[C@@H](Nc2nnc(-c3ccc(C#N)cc3O)c3c2C2CCC3CC2)C1] |
| 8a | 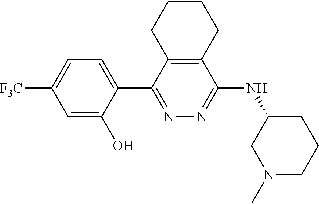 [SMILES: CN1CCC[C@@H](Nc2nnc(-c3ccc(C(F)(F)F)cc3O)c3c2CCCC3)C1] |
| 9a | 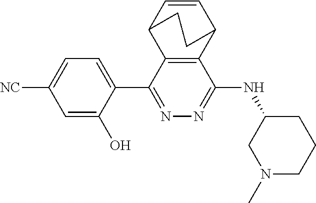 [SMILES: CN1CCC[C@@H](Nc2nnc(-c3ccc(C#N)cc3O)c3c2C2C=CC3CC2)C1] |
| 10a | 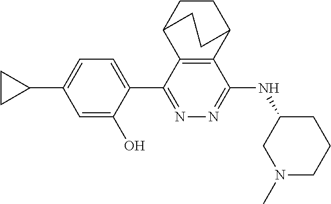 [SMILES: CN1CCC[C@@H](Nc2nnc(-c3ccc(C4CC4)cc3O)c3c2C2CCC3CC2)C1] |
| 11a | 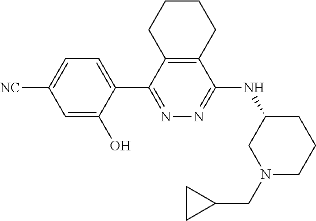 [SMILES: N#Cc1ccc(-c2nnc(N[C@@H]3CCCN(CC4CC4)C3)c3c2CCCC3)c(O)c1] |
| 12a | 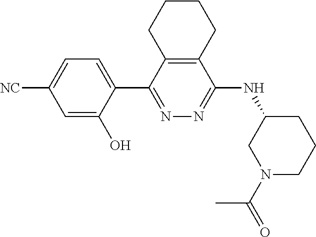 [SMILES: CC(=O)N1CCC[C@@H](Nc2nnc(-c3ccc(C#N)cc3O)c3c2CCCC3)C1] |
| 13a | 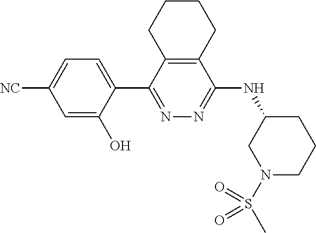 [SMILES: CS(=O)(=O)N1CCC[C@@H](Nc2nnc(-c3ccc(C#N)cc3O)c3c2CCCC3)C1] |
| 14a | 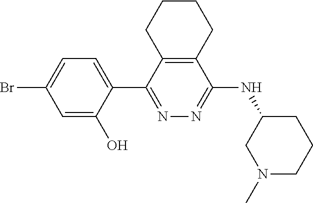 [SMILES: CN1CCC[C@@H](Nc2nnc(-c3ccc(Br)cc3O)c3c2CCCC3)C1] |
| 15a | 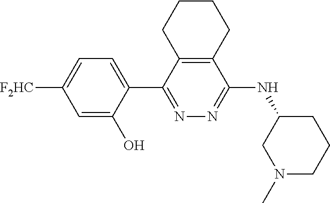 [SMILES: CN1CCC[C@@H](Nc2nnc(-c3ccc(C(F)F)cc3O)c3c2CCCC3)C1] |
| 16a | 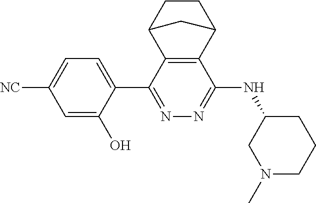 [SMILES: CN1CCC[C@@H](Nc2nnc(-c3ccc(C#N)cc3O)c3c2C2CCC3C2)C1] |
| 17a | 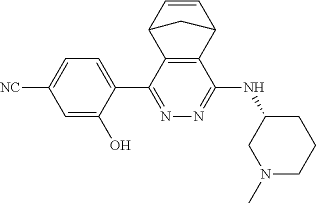 [SMILES: CN1CCC[C@@H](Nc2nnc(-c3ccc(C#N)cc3O)c3c2C2C=CC3C2)C1] |
| 18a | 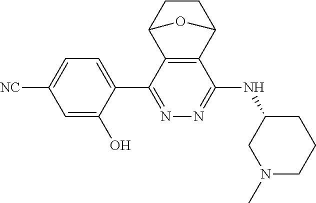 [SMILES: CN1CCC[C@@H](Nc2nnc(-c3ccc(C#N)cc3O)c3c2C2CCC3O2)C1] |
| 19a | 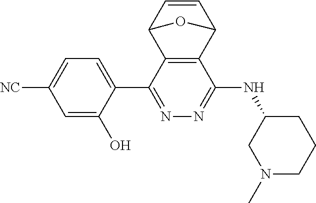 [SMILES: CN1CCC[C@@H](Nc2nnc(-c3ccc(C#N)cc3O)c3c2C2C=CC3O2)C1] |
| 20a | 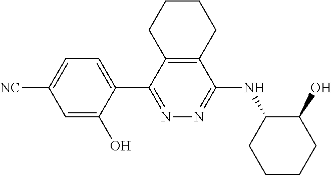 [SMILES: N#Cc1ccc(-c2nnc(N[C@H]3CCCC[C@@H]3O)c3c2CCCC3)c(O)c1] |
| 21a | 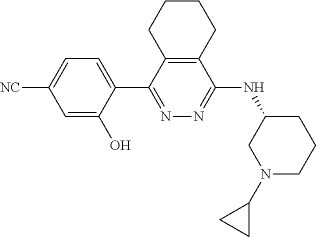 [SMILES: N#Cc1ccc(-c2nnc(N[C@@H]3CCCN(C4CC4)C3)c3c2CCCC3)c(O)c1] |
| 22a | 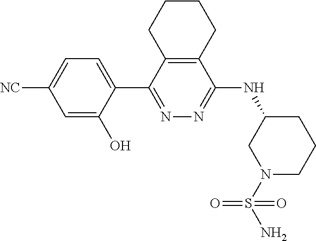 [SMILES: N#Cc1ccc(-c2nnc(N[C@@H]3CCCN(S(N)(=O)=O)C3)c3c2CCCC3)c(O)c1] |
| 23a | 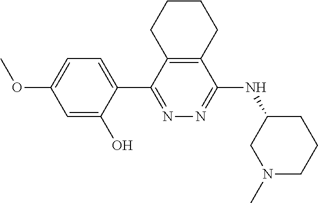 [SMILES: COc1ccc(-c2nnc(N[C@@H]3CCCN(C)C3)c3c2CCCC3)c(O)c1] |
| 24a | 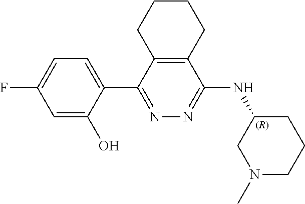 |
| 25a | 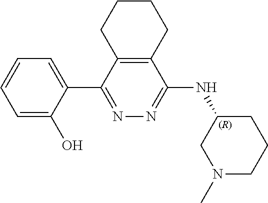 |
| 26a | 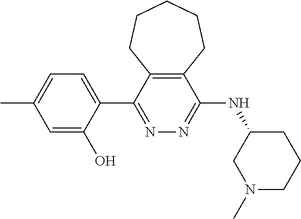 [SMILES: Cc1ccc(-c2nnc(N[C@@H]3CCCN(C)C3)c3c2CCCCC3)c(O)c1] |
| 27a | 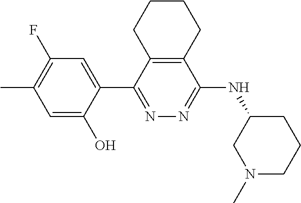 [SMILES: Cc1cc(O)c(-c2nnc(N[C@@H]3CCCN(C)C3)c3c2CCCC3)cc1F] |
| 28a | 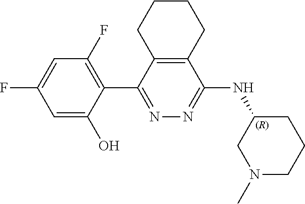 |
| 29a | 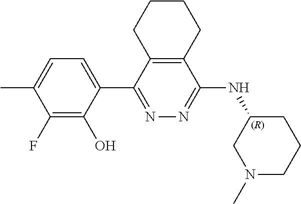 |
| 30a | 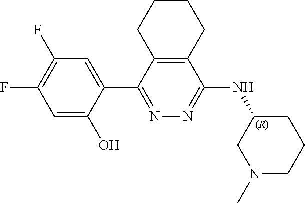 |
| 31a | 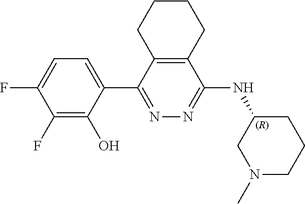 |
| 32a | 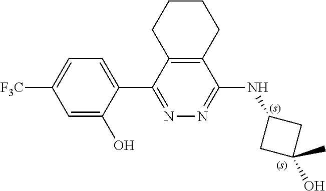 |
| 33a | 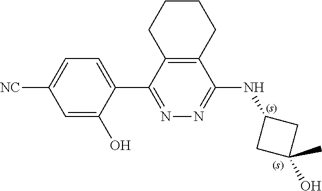 |
| 34a | 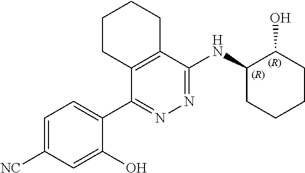 |

The technical solutions of the present invention also include a pharmaceutical composition comprising any of the above compounds, or the pharmaceutically acceptable salt, the stereoisomer or the tautomer thereof, and a pharmaceutically acceptable carrier.

The technical solutions of the present invention also include use of the compound of general formula (I) or general formula (II), or the pharmaceutically acceptable salt, the stereoisomer or the tautomer thereof, or the above pharmaceutical composition in the manufacture of a medicament for preventing and/or treating NLRP3 inflammasome-associated diseases.

The technical solutions of the present invention also include use of the above compound, or the pharmaceutically acceptable salt, the stereoisomer or the tautomer thereof, or the above pharmaceutical composition thereof in the manufacture of a medicament for preventing and/or treating inflammasome-associated diseases, immune diseases, inflammatory diseases, autoimmune diseases, or autoinflammatory diseases.

### DETAILED DESCRIPTION OF THE INVENTION

The “halogen” described herein refers to fluorine, chlorine, bromine, and iodine.

The “hydroxyl” described herein refers to —OH group.

The “cyano” described herein refers to —CN group.

The “amino” described herein refers to —NH₂ group.

The “carboxyl” described herein refers to —COOH group.

The “nitro” described herein refers to —NO₂ group.

The “C₁₋₆ alkyl” described herein refers to linear or branched alkyl derived by removing one hydrogen atom from a hydrocarbon moiety containing 1 to 6 carbon atoms, such as methyl, ethyl, n-propyl, isopropyl, n-butyl, isobutyl, sec-butyl, tert-butyl, n-pentyl, isopentyl, 2-methylbutyl, neopentyl, 1-ethylpropyl, n-hexyl, isohexyl, 4-methylpentyl, 3-methylpentyl, 2-methylpentyl, 1-methylpentyl, 3,3-dimethylbutyl, 2,2-dimethylbutyl, 1,1-dimethylbutyl, 1,2-dimethylbutyl, 1,3-dimethylbutyl, 2,3-dimethylbutyl, 2-ethylbutyl, and 1-methyl-2-methylpropyl.

The “ene” of “C₁₋₆ alkylene” described herein refers to a divalent group derived by removing two hydrogen atoms from a C₁₋₆ alkyl group.

The “haloC₁₋₆ alkyl” described herein refers to a C₁-C₆ alkyl group substituted with one or more halogen groups as defined above. Examples of haloC₁₋₆ alkyl include, but are not limited to, trifluoromethyl, difluoromethyl, fluoromethyl, trichloromethyl, 2,2,2-trifluoroethyl, 1,3-dibromopropan-2-yl, 3-bromo-2-fluoropropyl, and 1,4,4-trifluorobutan-2-yl.

The “C₁₋₆ alkoxy” described herein refers to a group in which the “C₁₋₆ alkyl” defined above is linked to a parent molecule via an oxygen atom, i.e., a “C₁₋₆ alkyl-O—” group, such as methoxy, ethoxy, n-propoxy, isopropoxy, n-butoxy, tert-butoxy, n-pentyloxy, neopentyloxy and n-hexyloxy.

The “haloC₁₋₆ alkoxy” described herein refers to a C₁-C₆ alkoxy group substituted with one or more halogen groups as defined above, examples of which include, but are not limited to, fluoromethoxy, chloromethoxy, trifluoromethoxy, trifluoroethoxy, fluoroethoxy, and fluoropropoxy.

The “C₂₋₆ alkenyl” described herein refers to linear or branched alkenyl derived by removing one hydrogen atom from an alkene moiety containing 2-6 carbon atoms and at least one carbon-carbon double bond, such as vinyl, 1-propenyl, 2-propenyl, 1-butenyl, 2-butenyl, 1,3-butadien-1-yl, 1-penten-3-yl, 2-penten-1-yl, 3-penten-1-yl, 3-penten-2-yl, 1,3-pentadien-1-yl, 1,4-pentadien-3-yl, 1-hexen-3-yl, and 1,4-hexadien-1-yl. Preferably, “C₂₋₆ alkenyl” contains one carbon-carbon double bond.

The “C₂₋₆ alkynyl” described herein refers to linear or branched alkynyl derived by removing one hydrogen atom from an alkyne moiety containing 2-6 carbon atoms and at least one carbon-carbon triple bond, such as ethynyl, propynyl, butynyl, pentynyl, and hexynyl. Preferably, “C₂₋₆ alkynyl” contains one carbon-carbon triple bond.

The “C₁₋₆ alkylamino”, “C₁₋₆ alkylcarbonylamino”, “C₁₋₆ alkylsulfonyl” and “aminocarbonyl” described herein refer to C₁₋₆ alkyl-NH— group, C₁₋₆ alkyl-C(O)—NH— group, C₁₋₆ alkyl-S(O)₂— group, and NH₂—C(O)— group, respectively.

The “5-12 membered ring” described herein includes carbon rings or heterocyclic rings which may be chemically formed, such as 5-12 membered cycloalkyl, 5-7 membered cycloalkyl, 5-12 membered cycloalkenyl, 5-7 membered cycloalkenyl, 6-12 membered fused cycloalkyl, 5-12 membered heterocyclyl, 5-7 membered heterocyclyl, 6-12 membered fused heterocycle, aryl, 5-12 membered heteroaryl, 8-12 membered fused heteroaryl, and 5-7 membered heteroaryl.

The “3-12 membered cycloalkyl” described herein refers to a monovalent group or (as required) divalent group (e.g, 5-12 membered cycloalkyl) derived from 3-12 membered cycloalkane, which may be a monocyclic, bicyclic or fused cycloalkyl system. Unless otherwise specified, a certain membered cycloalkyl group (e.g., 5-12 membered cycloalkyl, 5-8 membered cycloalkyl, 5-7 membered cycloalkyl, 3-7 membered cycloalkyl, 3-6 membered cycloalkyl, or 4-6 membered cycloalkyl) includes all monocyclic, fused rings (e.g., 6-12 membered fused cycloalkyl) which may be formed, including cases where they are fused in the ortho-fused, spiro- and bridged form.

A monocyclic system is typically a cyclic hydrocarbon group containing 3 to 12 carbon atoms (such as 3 to 8 or 3 to 6 carbon atoms). Examples of cycloalkyl include, but are not limited to: cyclopropyl, cyclobutyl, cyclopentyl, cyclohexyl, cycloheptyl, cyclooctyl, cyclopentyl-1,3-diyl, cyclohexyl-1,4-diyl, and cycloheptyl-1,4-diyl. Fused cycloalkyl includes ortho-fused cycloalkyl, bridged cycloalkyl and spiro-cycloalkyl. Ortho-fused cycloalkyl may be 6-11 membered ortho-fused cycloalkyl (such as 7-10 membered ortho-fused cycloalkyl), and the representative examples include, but are not limited to, bicyclo[3.1.1]heptane, bicyclo[2.2.1]heptane, bicyclo[2.2.2]octane, bicyclo[3.2.2]nonane, bicyclo[3.3.1]nonane and bicyclo[4.2.1]nonyl. Spiro-cycloalkyl may be 7-12 membered spiro-cycloalkyl (such as 7-11 membered spiro-cycloalkyl), examples of which include, but are not limited to

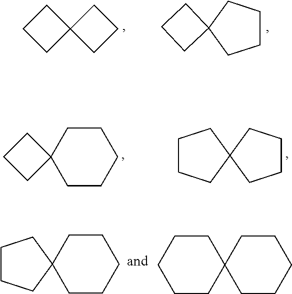

groups. Bridged cycloalkyl may be 6-10 membered bridged cycloalkyl (such as 7-10 membered bridged cycloalkyl), examples of which include, but are not limited to:

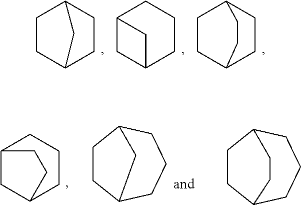

groups.

The “3-7 membered cycloalkyl” described herein refers to a monovalent group or (as required) divalent group derived from 3-7 membered cycloalkane. The “3-7 membered cycloalkyl” may be 3, 4, 5, 6, or 7 membered cycloalkyl, and examples of the 3-7 membered cycloalkyl include cyclopropyl, cyclobutyl, cyclopentyl, and cyclohexyl.

The “cycloalkenyl” described herein refers to a group obtained by having at least one double bond in the above cycloalkyl. It may be, for example, “3-12 membered cycloalkenyl”, i.e., may have 3, 4, 5, 6, 7, 8, 9, 10, 11 or 12 ring-forming carbon atoms. Unless otherwise specified, a certain membered cycloalkenyl encompasses all possible monocyclic or fused cycloalkenyls (including ortho-fused, spiro and bridged). Cycloalkenyl may be 3-12 membered cycloalkenyl, 3-8 membered cycloalkenyl, 5-8 membered cycloalkenyl, 5-7 membered cycloalkenyl, 4-6 membered cycloalkenyl, 7-11 membered spiro-cycloalkenyl, 7-11 membered ortho-fused cycloalkenyl, 6-11 membered bridged cycloalkenyl, etc. Examples of cycloalkenyl include, but are not limited to, cyclobutenyl, cyclopentenyl, cyclopentadienyl, cyclohexenyl, 1,4-cyclohexadien-1-yl, cycloheptenyl, 1,4-cycloheptadien-1-yl, cyclooctenyl and 1,5-cyclooctadien-1-yl.

The “5-7 membered cycloalkenyl” described herein refers to a group obtained by having at least one double bond in a 5-7 membered cycloalkyl group, such as cyclobutenyl, cyclopentenyl, cyclohexenyl, and cycloheptenyl.

The “3-14 membered heterocyclyl” described herein refers to a monovalent group or (as required) divalent group derived from 3-14 membered heterocycloalkane, i.e., a non-aromatic cyclic group obtained by substituting at least one ring carbon atom of 3-14 membered heterocycloalkane with a heteroatom selected from O, S, S(O), S(O)₂, C(O) and N, which preferably contains 1-3 heteroatoms. The “3-14 membered heterocyclyl” (e.g., 5-14 membered heterocyclyl, or 5-12 membered heterocyclyl) includes monocyclic heterocyclyl, bicyclic heterocyclyl systems, or fused heterocyclic systems in which one or more rings may be saturated or partially saturated, but does not include aromatic rings. Unless otherwise specified, a certain membered heterocyclyl group (e.g., 3-8 membered heterocyclyl, 3-7 membered heterocyclyl, 5-8 membered heterocyclyl, 5-7 membered heterocyclyl, 5-6 membered heterocyclyl, 4-6 membered heterocyclyl, or 6 membered heterocyclyl) includes all monocyclic, fused ring (including fusion in the ortho-fused, spiro- and bridged form), and saturated and partially saturated cases which may be formed.

Monocyclic heterocyclyl may be 3-8 membered heterocyclyl (such as 5-7 membered heterocyclyl, 3-7 membered heterocyclyl, 4-7 membered heterocyclyl, or 5-6 membered heterocyclyl), 3-8 membered nitrogen-containing heterocyclyl (such as 4-7 membered nitrogen-containing heterocyclyl, or 5-6 membered nitrogen-containing heterocyclyl), or 3-8 membered saturated heterocyclyl (such as 5-6 membered saturated heterocyclyl), examples of which include, but are not limited to, azacyclopropyl, ozacyclopropyl, thiocyclopropyl, azacyclobutyl, oxacyclobutyl, thiocyclobutyl, tetrahydrofuranyl, tetrahydropyrrolyl, tetrahydrothienyl, imidazolidinyl, pyrazolidinyl, 1,2-oxazolidinyl, 1,3-oxazolidinyl, 1,2-thiazolidinyl, 1,3-thiazolidinyl, tetrahydro-2H-pyranyl, tetrahydro-2H-thiopyranyl, piperidinyl, piperazinyl, morpholinyl, 1,4-dioxanyl, 1,4-oxathianyl, 4,5-dihydroisoxazolyl, 4,5-dihydrooxazolyl, 2,5-dihydrooxazolyl, 2,3-dihydrooxazolyl, 3,4-dihydro-2H-pyrrolyl, 2,3-dihydro-1H-pyrrolyl, 2,5-dihydro-1H-imidazolyl, 4,5-dihydro-1H-imidazolyl, 4,5-dihydro-1H-pyrazolyl, 4,5-dihydro-3H-pyrazolyl, 4,5-dihydrothiazolyl, 2,5-dihydrothiazolyl, 2H-pyranyl, 4H-pyranyl, 2H-thiopyranyl, 4H-thiopyranyl, 2,3,4,5-tetrahydropyridyl, 1,2-isoxazolyl, 1,4-isoxazolyl, and 6H-1,3-oxazinyl.

Fused heterocyclyl (e.g., 6-12 membered fused heterocyclyl) includes ortho-fused heterocyclyl, spiro-heterocyclyl, and bridged heterocyclyl, which may be saturated, partially saturated or unsaturated, but non-aromatic. Fused heterocyclyl may be a 5-6 membered monocyclic heterocyclyl ring which is fused to a benzene ring, 5-6 membered monocyclic cycloalkyl, 5-6 membered monocyclic cycloalkenyl, 5-6 membered monocyclic heterocyclyl or 5-6 membered monocyclic heteroaryl.

The ortho-fused heterocyclyl may be 6-12 membered ortho-fused heterocyclyl (such as 6-11 membered ortho-fused heterocyclyl or 7-10 membered ortho-fused heterocyclyl), 6-11 membered saturated ortho-fused heterocyclyl or 6-11 membered nitrogen-containing ortho-fused heterocyclyl, and the representative examples thereof include, but are not limited to: 3-azabicyclo[3.1.0]hexyl, 3,6-diazabicyclo[3.2.0]heptyl, 3,8-diazabicyclo[4.2.0]octyl, 3,7-diazabicyclo[4.2.0]octyl, octahydropyrrolo[3,4-c]pyrrolyl, octahydropyrrolo[3,4-b]pyrrolyl, octahydropyrrolo[3,4-b][1,4]oxazinyl, octahydro-1H-pyrrolo[3,4-c]pyridyl, 2,3-dihydrobenzofuran-2-yl, 2,3-dihydrobenzofuran-3-yl, indolin-1-yl, indolin-2-yl, indolin-3-yl, 2,3-dihydrobenzothiophen-2-yl, octahydro-1H-indolyl, and octahydrobenzofuranyl.

The spiro-heterocyclyl may be 6-12 membered spiro-heterocyclyl (such as 7-12 membered spiro-heterocyclyl), 7-12 membered saturated spiro-heterocyclyl or 7-12 membered nitrogen-containing spiro-heterocyclyl, and the examples include, but are not limited to:

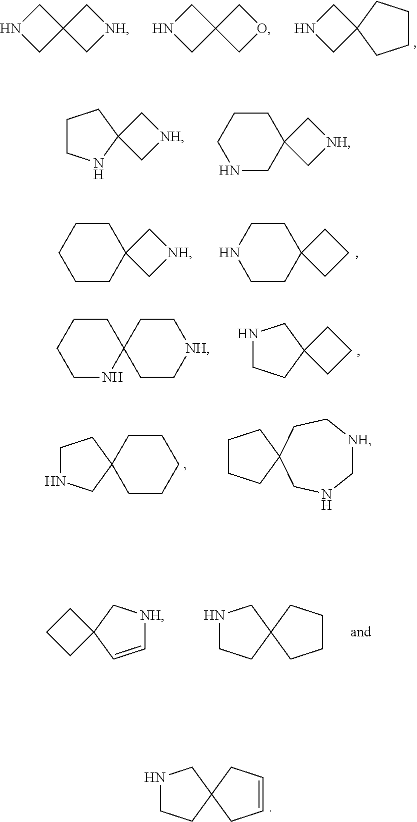

The bridged heterocyclyl may be 6-12 membered bridged heterocyclyl (such as 6-10 membered bridged heterocyclyl (e.g., 6-10 membered nitrogen-containing bridged heterocyclyl, particularly 7-membered nitrogen-containing bridged heterocyclyl), or 7-10 membered bridged heterocyclyl), examples of which include, but are not limited to:

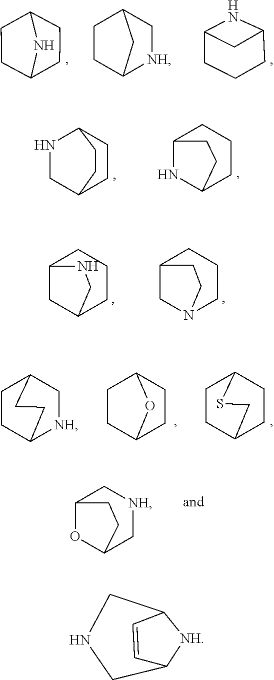

The “aryl” described herein refers to a monovalent or (as required) divalent cyclic aromatic group containing 6-14 carbon atoms derived from aromatic carbocyclic hydrocarbon, including phenyl, naphthyl, phenanthryl, etc.

The “5-14 membered heteroaryl” described herein refers to an aromatic 5-14 membered cyclic group in which at least one ring carbon atom is substituted with a heteroatom selected from O, S and N, which may be 5, 6, 7, 8, 9, 10, 11, 12, 13 or 14 membered heteroaryl and preferably contains 1-3 heteroatoms. Moreover, the case where carbon atoms or sulfur atoms are oxidized or nitridized is included. For example, carbon atoms are substituted with C(O) and sulfur atoms are substituted with S(O) or S(O)₂. Heteroaryl includes monocyclic heteroaryl and fused heteroaryl. Unless otherwise specified, a certain membered heteroaryl includes all possibly formed monocyclic, polycyclic, fully aromatic and partially aromatic cases. Monocyclic heteroaryl may be 5-7 membered heteroaryl (such as 5-6 membered heteroaryl), examples of which include, but are not limited to, furanyl, imidazolyl, isoxazolyl, thiazolyl, isothiazolyl, oxadiazolyl, oxazolyl, pyridyl, pyridazinyl, pyrimidinyl, pyrazinyl, pyrazolyl, pyrrolyl, tetrazolyl, thiadiazolyl, thienyl, triazolyl and triazinyl.

In some embodiments, fused heteroaryl refers to a group which is formed by fusing a monocyclic heteroaromatic ring to phenyl, cycloalkenyl, heteroaryl, cycloalkyl or heterocyclyl. In some embodiments, fused heteroaryl (such as 8-14 membered fused heteroaryl) may be 8-14 membered ortho-fused heteroaryl (such as 9-10 membered ortho-fused heteroaryl), and the examples include, but are not limited to, benzimidazolyl, benzofuranyl, benzothienyl, benzoxadiazolyl, benzothiadiazolyl, benzothiazolyl, cinnolinyl, 5,6-dihydroquinolin-2-yl, 5,6-dihydroisoquinolin-1-yl, furopyridinyl, indazolyl, indolyl, isoindolyl, isoquinolinyl, naphthyridinyl, purinyl, quinolinyl, 5,6,7,8-tetrahydroquinolin-2-yl, 5,6,7,8-tetrahydroquinolinyl, 5,6,7,8-tetrahydroquinolin-4-yl, 5,6,7,8-tetrahydroisoquinolin-1-yl, thienopyridyl, 4,5,6,7-tetrahydro[c][1,2,5]oxadiazolyl and 6,7-dihydro[c][1,2,5]oxadiazol-4(5H)keto.

The “pharmaceutically acceptable salt” described herein refers to a pharmaceutically acceptable addition salt of acid and base and a solvate. Such pharmaceutically acceptable salts include salts of the following acids: hydrochloric acid, phosphoric acid, hydrobromic acid, sulfuric acid, sulfurous acid, formic acid, toluenesulfonic acid, methanesulfonic acid, nitric acid, benzoic acid, citric acid, tartaric acid, maleic acid, hydroiodic acid, alkanoic acid (such as acetic acid, HOOC—(CH₂)n-COOH (wherein n is 0-4)), etc. Such pharmaceutically acceptable salts further include salts of the following bases: sodium, potassium, calcium, ammonium, etc. Those skilled in the art know a variety of pharmaceutically acceptable non-toxic addition salts.

The term “optionally” means that the subsequently described event may or may not occur, and that the description includes both the occurrence and non-occurrence of the event.

All numerical ranges described herein include both endpoints of the ranges, all integers within the ranges, and subranges formed by these integers. For example, “3-7 membered” includes 3, 4, 5, 6, and 7 membered; “0-4” includes 0, 1, 2, 3, and 4.

The “stereoisomer” of the compound described herein refers to an isomer resulting from the different spatial arrangement of the atoms in a molecule. An enantiomer will be produced when an asymmetric carbon atom is present in a compound, or a cis-trans isomer will be produced when a carbon-carbon double bond or a ring structure is present in a compound.

The term “tautomer” refers to a particular functional group isomer in which different functional group isomers are in dynamic equilibrium and are rapidly transformed into each other. For example, in the presence of a ketone or oxime, a tautomer will be produced, and representative examples are: keto-enol tautomers, phenol-keto tautomers, nitroso-oxime tautomers, imine-enamine tautomers, etc.

All enantiomers, diastereomers, racemates, cis-trans isomers, tautomers, geometric isomers, and epimers of all of the compounds, and mixtures thereof are included in the scope of the present invention.

In the chemical configuration of the compound of the present invention, the bond “/” represents an unspecified configuration, namely, if a chiral isomer is present in a chemical structure, the bond “ [symbol] ” may be “ [symbol] ” or “ [symbol] ”, or includes both “ [symbol] ” and “ [symbol] ” In the chemical structure of the compound of the present invention, “ [symbol] ” indicates the point linked to the parent molecule.

The present invention provides a compound of general formula (I), or a pharmaceutically acceptable salt, a stereoisomer or a tautomer thereof:

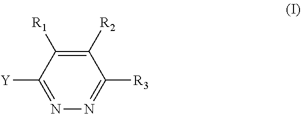

wherein,
R₁ is selected from hydrogen, hydroxyl, amino, carboxyl, cyano, nitro, halogen, C₁₋₆ alkyl, haloC₁₋₆ alkyl, C₁₋₆ alkoxy, haloC₁₋₆ alkoxy, C₂₋₆ alkenyl, C₂₋₆ alkynyl, 3-7 membered heterocyclyl, 3-7 membered cycloalkyl, aryl, and 5-7 membered heteroaryl;
R₂ is selected from hydrogen, hydroxyl, amino, carboxyl, cyano, nitro, halogen, C₁₋₆ alkyl, haloC₁₋₆ alkyl, C₁₋₆ alkoxy, haloC₁₋₆ alkoxy, C₂₋₆ alkenyl, C₂₋₆ alkynyl, 3-7 membered heterocyclyl, 3-7 membered cycloalkyl, aryl, and 5-7 membered heteroaryl;
R₁ and R₂ are respectively optionally substituted with 1-3 substituents selected from hydroxyl, amino, carboxyl, cyano, nitro, halogen, carbonyl, C₁₋₆ alkyl, haloC₁₋₆ alkyl, C₁₋₆ alkoxy, haloC₁₋₆ alkoxy, 3-7 membered heterocyclyl, 3-7 membered cycloalkyl, aryl, and 5-7 membered heteroaryl;
or,
R₁ and R₂, together with the carbon atom to which they are attached, form a 5-12 membered ring A, wherein the 5-12 membered ring A is optionally substituted with 1-4 substituents selected from hydroxyl, amino, carboxyl, cyano, nitro, halogen, carbonyl, C₁₋₆ alkyl, —NH—C₁₋₆ alkyl, haloC₁₋₆ alkyl, C₁₋₆ alkoxy, haloC₁₋₆ alkoxy, 3-7 membered heterocyclyl, 3-7 membered cycloalkyl, aryl, 5-7 membered heteroaryl, and C₁₋₆ alkylsulfonyl;
R₃ is selected from —(C₁₋₆ alkylene)₀₋₂-NR₄R₅, —(C₁₋₆ alkylene)₀₋₂NR₄—COR₅, and —(C₁₋₆ alkylene)₀₋₂-CO—NR₄-R₅; R₄ is selected from hydrogen and C₁₋₆ alkyl; R₅ is selected from 3-7 membered heterocyclyl, 3-7 membered cycloalkyl, aryl, and 5-7 membered heteroaryl; R₅ is optionally substituted with 1-4 substituents selected from halogen, cyano, amino, hydroxyl, C₁₋₆ alkyl, haloC₁₋₆ alkyl, C₁₋₆ alkoxy, 3-7 membered heterocyclyl, 3-7 membered cycloalkyl, aryl, and 5-7 membered heteroaryl;
Y is selected from aryl, 5-14 membered heteroaryl, 3-14 membered heterocyclyl, and 3-12 membered cycloalkyl, and Y is optionally substituted with 1-3 substituents selected from halogen, cyano, amino, hydroxyl, carbonyl, C₁₋₆ alkyl, haloC₁₋₆ alkyl, C₁₋₆ alkoxy, 3-7 membered heterocyclyl, 3-7 membered cycloalkyl, aryl, 5-7 membered heteroaryl, C₁₋₆ alkylamino, C₁₋₆ alkylcarbonylamino, C₁₋₆ alkylsulfonyl, and aminocarbonyl;
with the proviso that:
when Y is hydroxyl-substituted phenyl, R₁ and R₂ are not hydrogen, methyl, cyano, and trifluoromethyl;
when ring A is absent, and R₃ is —NH—R₅, R₅ is not

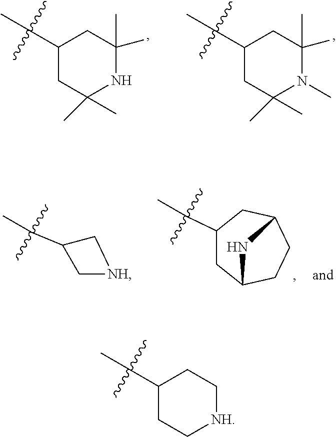

In some embodiments, R₁ and R₂, together with the carbon atom to which they are attached, form a 5-12 membered ring A, wherein the 5-12 membered ring A is optionally substituted with —N(C₁₋₆ alkyl)₂.

In some embodiments, R₃ is —(C₁₋₆ alkylene)₀₋₂-O—R₅.

In some embodiments, R₅ is optionally substituted with a substituent selected from C₂₋₆ alkenylcarbonyl, sulfonyl, and C₁₋₆ alkylcarbonyl.

In some embodiments, R₅ is optionally substituted with carboxyl.

In some embodiments, Y is optionally substituted with a substituent selected from C₁₋₆ alkylaminocarbonyl, and sulfonyl.

In some embodiments, Y is optionally substituted with a substituent selected from C₁₋₆ alkylthio and C₁₋₆ alkylsulfinyl.

In some embodiments, when R₅ is substituted, the substituents on R₅, which are C₁₋₆ alkyl, haloC₁₋₆ alkyl, C₁₋₆ alkoxy, 3-7 membered heterocyclyl, 3-7 membered cycloalkyl, aryl, 5-7 membered heteroaryl, and sulfonyl, are optionally substituted with 1-3 substituents selected from halogen, cyano, amino, hydroxyl, carbonyl, C₁₋₆ alkyl, and 3-6 membered cycloalkyl;
when Y is substituted,
the substituents on Y, which are C₁₋₆ alkyl, haloC₁₋₆ alkyl, C₁₋₆ alkoxy, 3-7 membered heterocyclyl, 3-7 membered cycloalkyl, aryl, 5-7 membered heteroaryl, and sulfonyl, are optionally substituted with 1-3 substituents selected from halogen, cyano, amino, hydroxyl, carbonyl, C₁₋₆ alkyl, and 3-6 membered cycloalkyl.

In some embodiments, R₁ and R₂, together with the carbon atom to which they are attached, form a 5-12 membered ring A, wherein the 5-12 membered ring A is optionally substituted with —N(C₁₋₆ alkyl)₂; R₅ is optionally substituted with a substituent selected from C₂₋₆ alkenylcarbonyl, sulfonyl, and C₁₋₆ alkylcarbonyl; Y is optionally substituted with a substituent selected from C₁₋₆ alkylaminocarbonyl and sulfonyl; the substituents on R₅ and Y, which are C₁₋₆ alkyl, haloC₁₋₆ alkyl, C₁₋₆ alkoxy, 3-7 membered heterocyclyl, 3-7 membered cycloalkyl, aryl, 5-7 membered heteroaryl, and sulfonyl, are optionally substituted with 1-3 substituents selected from halogen, cyano, amino, hydroxyl, carbonyl, C₁₋₆ alkyl, and 3-6 membered cycloalkyl.

In some embodiments, ring A is selected from 5-12 membered cycloalkyl, 5-12 membered cycloalkenyl, 5-12 membered heterocyclyl, aryl, and 5-12 membered heteroaryl.

In some embodiments, ring A is selected from 5-8 membered cycloalkyl, 5-8 membered cycloalkenyl, 5-8 membered heterocyclyl, phenyl, and 5-8 membered heteroaryl.

In some embodiments, ring A is selected from phenyl, and 5-6 membered heteroaryl, 5-8 membered cycloalkyl, 5-8 membered cycloalkenyl and 5-8 membered heterocyclyl containing 1-2 heteroatoms selected from O, S, and N.

In some embodiments, ring A is optionally substituted with 1-4 substituents selected from hydroxyl, amino, carboxyl, cyano, nitro, halogen, carbonyl, C₁₋₆ alkyl, —NH—C₁₋₆ alkyl, haloC₁₋₆ alkyl, C₁₋₆ alkoxy, haloC₁₋₆ alkoxy, 3-7 membered heterocyclyl, 3-7 membered cycloalkyl, aryl, 5-7 membered heteroaryl, C₁₋₆ alkylsulfonyl, and —N(C₁₋₆ alkyl)₂.

In some embodiments, ring A is unsubstituted.

In some embodiments, ring A is substituted with 1-2 substituents selected from cyano, halogen, C₁₋₆ alkyl, —NH—C₁₋₆ alkyl, haloC₁₋₆ alkyl, C₁₋₆ alkoxy, 3-7 membered cycloalkyl, 5-7 membered heteroaryl, C₁₋₆ alkylsulfonyl, and —N(C₁₋₆ alkyl)₂;

In some embodiments, ring A is selected from 5-8 membered cycloalkyl, 5-8 membered cycloalkenyl, 5-8 membered heterocyclyl, phenyl, and 5-6 membered heteroaryl; ring A is optionally substituted with 1-2 substituents selected from cyano, halogen, C₁₋₆ alkyl, —NH—C₁₋₆ alkyl, haloC₁₋₆ alkyl, C₁₋₆ alkoxy, 3-7 membered cycloalkyl, 5-7 membered heteroaryl, C₁₋₆ alkylsulfonyl, and —N(C₁₋₆ alkyl)₂.

In some embodiments, the present invention relates to the aforementioned compound, or the pharmaceutically acceptable salt, the stereoisomer or the tautomer thereof, which has the structure of general formula (II):

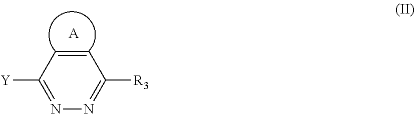

wherein ring A is selected from 5-7 membered cycloalkenyl, 5-7 membered cycloalkyl, 5-7 membered heterocyclyl, phenyl, and 5-7 membered heteroaryl; ring A is optionally substituted with 1-4 substituents selected from hydroxyl, amino, carboxyl, cyano, nitro, halogen, C₁₋₆ alkyl, —NH—C₁₋₆ alkyl, haloC₁₋₆ alkyl, C₁₋₆ alkoxy, haloC₁₋₆ alkoxy, 3-7 membered heterocyclyl, 3-7 membered cycloalkyl, aryl, 5-7 membered heteroaryl, and C₁₋₆ alkylsulfonyl. Furthermore, ring A is optionally substituted with —N(C₁₋₆ alkyl)₂.

In some embodiments, the present invention relates to the aforementioned compound, or the pharmaceutically acceptable salt, the stereoisomer or the tautomer thereof, wherein ring A is selected from phenyl and 5-7 membered heteroaryl; ring A is optionally substituted with 1-4 substituents selected from hydroxyl, amino, carboxyl, cyano, nitro, halogen, C₁₋₆ alkyl, —NH—C₁₋₆ alkyl, haloC₁₋₆ alkyl, C₁₋₆ alkoxy, haloC₁₋₆ alkoxy, 3-7 membered heterocyclyl, 3-7 membered cycloalkyl, and C₁₋₆ alkylsulfonyl. Furthermore, ring A is optionally substituted with —N(C₁₋₆ alkyl)₂.

In some embodiments, the present invention relates to the aforementioned compound, or the pharmaceutically acceptable salt, the stereoisomer or the tautomer thereof, wherein ring A is selected from

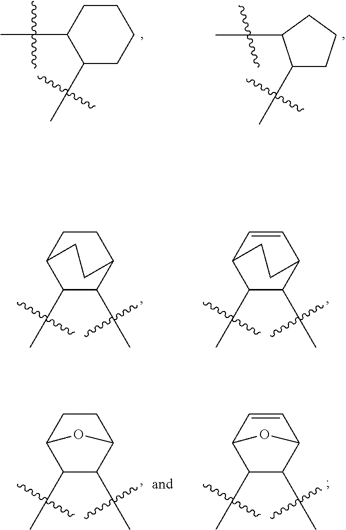

ring A is optionally substituted with 1-4 substituents selected from hydroxyl, amino, carboxyl, cyano, nitro, halogen, C₁₋₆ alkyl, —NH—C₁₋₆ alkyl, haloC₁₋₆ alkyl, C₁₋₆ alkoxy, haloC₁₋₆ alkoxy, 3-7 membered heterocyclyl, 3-7 membered cycloalkyl, C₁₋₆ alkylsulfonyl, and —N(C₁₋₆ alkyl)₂.

In some embodiments, ring A is selected from

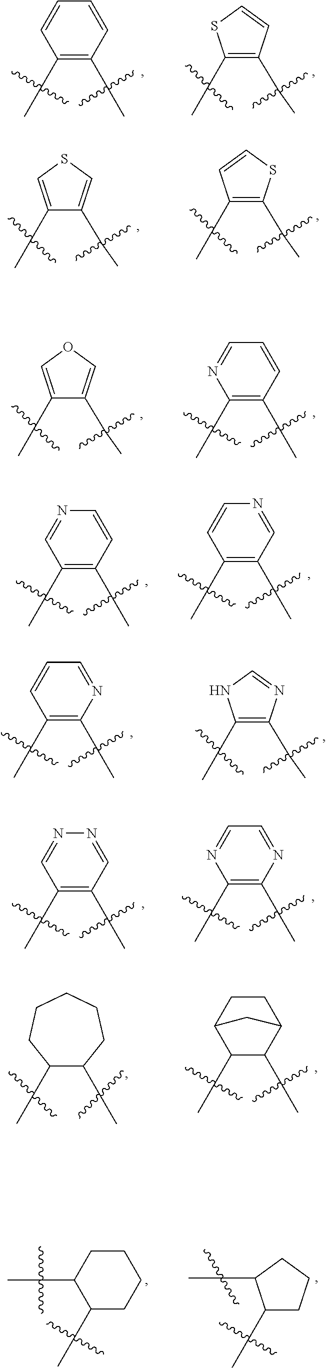

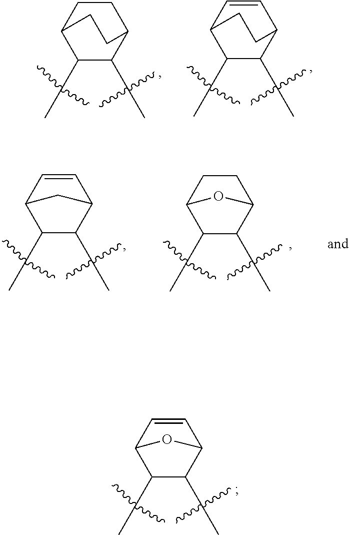 [SMILES: *C1C2CCC(CC2)C1* ; *C1C2C=CC(CC2)C1* ; *C1C2C=CC(C2)C1* ; *C1C2CCC(O2)C1* ; *C1C2C=CC(O2)C1*]

ring A is optionally substituted with 1-2 substituents selected from cyano, halogen, C₁₋₆ alkyl, —NH—C₁₋₆ alkyl, haloC₁₋₆ alkyl, C₁₋₆ alkoxy, 3-7 membered cycloalkyl, 5-7 membered heteroaryl, C₁₋₆ alkylsulfonyl, and —N(C₁₋₆ alkyl)₂.

In some embodiments, ring A is

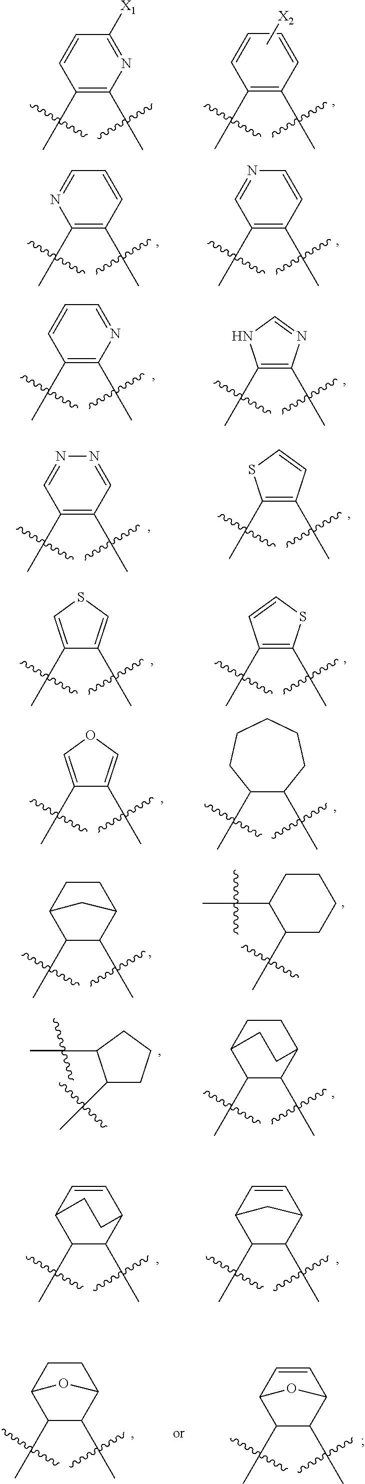

X₁ is selected from H, CH₃, F, Cl, Br, —OCH₃, CN, —CF₃, —NHCH₃, and

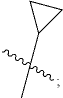

X₂ is selected from H, CH₃, F, Cl, Br, —OCH₃, CN, —CF₃, —NHCH₃,

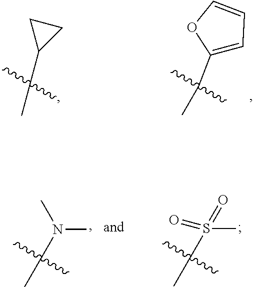

preferably, ring A is further unsubstituted.

In some embodiments, Y is selected from aryl, 5-14 membered heteroaryl, 3-14 membered heterocyclyl, and 3-12 membered cycloalkyl, and Y is optionally substituted with 1-3 substituents selected from halogen, cyano, amino, hydroxyl, carbonyl, C₁₋₆ alkyl, haloC₁₋₆ alkyl, C₁₋₆ alkoxy, 3-7 membered heterocyclyl, 3-7 membered cycloalkyl, aryl, 5-7 membered heteroaryl, C₁₋₆ alkylamino, C₁₋₆ alkylcarbonylamino, C₁₋₆ alkylsulfonyl, aminocarbonyl, C₁₋₆ alkylaminocarbonyl, sulfonyl, C₁₋₆ alkylthio, and C₁₋₆ alkylsulfinyl.

In some embodiments, the present invention relates to the aforementioned compound, or the pharmaceutically acceptable salt, the stereoisomer or the tautomer thereof,
wherein,
Y is selected from phenyl, 5-7 membered heteroaryl, 3-8 membered heterocyclyl, and 3-7 membered cycloalkyl; Y is optionally substituted with 1-3 substituents selected from halogen, cyano, amino, hydroxyl, carbonyl, C₁₋₆ alkyl, haloC₁₋₆ alkyl, C₁₋₆ alkoxy, 3-7 membered heterocyclyl, 3-7 membered cycloalkyl, C₁₋₆ alkylamino, C₁₋₆ alkylcarbonylamino, C₁₋₆ alkylsulfonyl, and aminocarbonyl;
with the proviso that:
when Y is hydroxyl-substituted phenyl, R₁ and R₂ are not hydrogen, methyl, cyano, and trifluoromethyl;
when ring A is absent, and R₃ is —NH—R₅, R₅ is not

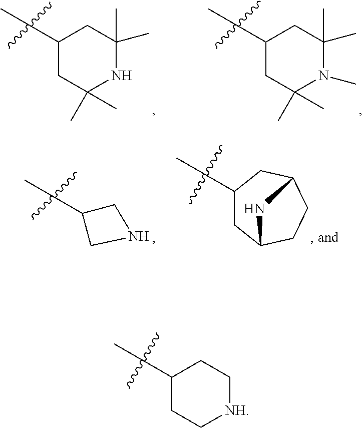 [SMILES: *C1CC(C)(C)NC(C)(C)C1 ; *C1CC(C)(C)N(C)C(C)(C)C1 ; *C1CNC1 ; *C1C[C@H]2CC[C@@H](C1)N2 ; *C1CCNCC1]

In some embodiments, Y is optionally substituted with a substituent of C₁₋₆ alkylaminocarbonyl or sulfonyl.

In some embodiments, when Y is substituted, C₁₋₆ alkyl, haloC₁₋₆ alkyl, C₁₋₆ alkoxy, 3-7 membered heterocyclyl, 3-7 membered cycloalkyl or sulfonyl on Y is optionally substituted with 1-3 substituents selected from halogen, cyano, amino, hydroxyl, carbonyl, and C₁₋₆ alkyl.

In some embodiments, Y is selected from phenyl, 5-8 membered heteroaryl, 3-8 membered heterocyclyl, and 3-7 membered cycloalkyl.

In some embodiments, Y is selected from phenyl and 5-7 membered heteroaryl.

In some embodiments, Y is selected from phenyl, and 5-6 membered heteroaryl containing 1-2 N heteroatoms.

In some embodiments, Y is optionally substituted with 1-3 substituents selected from halogen, cyano, amino, hydroxyl, carbonyl, C₁₋₆ alkyl, haloC₁₋₆ alkyl, C₁₋₆ alkoxy, 3-7 membered heterocyclyl, 3-7 membered cycloalkyl, aryl, 5-7 membered heteroaryl, C₁₋₆ alkylamino, C₁₋₆ alkylcarbonylamino, C₁₋₆ alkylsulfonyl, aminocarbonyl, C₁₋₆ alkylaminocarbonyl, sulfonyl, C₁₋₆ alkylthio, and C₁₋₆ alkylsulfinyl.

In some embodiments, when Y is substituted, the substituents on Y, which are C₁₋₆ alkyl, haloC₁₋₆ alkyl, C₁₋₆ alkoxy, 3-7 membered heterocyclyl, 3-7 membered cycloalkyl, aryl, 5-7 membered heteroaryl, and sulfonyl, are optionally substituted with 1-3 substituents selected from halogen, cyano, amino, hydroxyl, carbonyl, C₁₋₆ alkyl, and 3-6 membered cycloalkyl.

In some embodiments, Y is substituted with 1-3 substituents selected from halogen, cyano, hydroxyl, amino, C₁₋₆ alkyl, C₁₋₆ alkoxy, hydroxyl-substituted C₁₋₆ alkyl, haloC₁₋₆ alkyl, aminocarbonyl, C₁₋₆ alkylamino, C₁₋₆ alkylcarbonylamino, C₁₋₆ alkylaminocarbonyl, 3-7 membered heterocyclyl, 3-7 membered cycloalkyl, 5-7 membered heteroaryl, C₁₋₆ alkylsulfonyl, C₁₋₆ alkylthio, and C₁₋₆ alkylsulfinyl.

In some embodiments, Y is substituted with cyano and is optionally substituted with 1-2 substituents selected from halogen, cyano, amino, hydroxyl, carbonyl, C₁₋₆ alkyl, haloC₁₋₆ alkyl, C₁₋₆ alkoxy, 3-7 membered heterocyclyl, 3-7 membered cycloalkyl, C₁₋₆ alkylamino, C₁₋₆ alkylcarbonylamino, C₁₋₆ alkylsulfonyl, aminocarbonyl, C₁₋₆ alkylaminocarbonyl, and sulfonyl; the C₁₋₆ alkyl, haloC₁₋₆ alkyl, C₁₋₆ alkoxy, 3-7-membered heterocyclyl, 3-7-membered cycloalkyl and sulfonyl are optionally substituted with 1-3 substituents selected from halogen, cyano, amino, hydroxyl, carbonyl, and C₁₋₆ alkyl.

In some embodiments, Y is substituted with 1 cyano and is optionally substituted with 1-2 substituents selected from halogen, hydroxyl, amino, C₁₋₆ alkyl, C₁₋₆ alkoxy, hydroxyl-substituted C₁₋₆ alkyl, haloC₁₋₆ alkyl, aminocarbonyl, C₁₋₆ alkylamino, C₁₋₆ alkylcarbonylamino, C₁₋₆ alkylaminocarbonyl, 3-7 membered heterocyclyl, 3-7 membered cycloalkyl, 5-7 membered heteroaryl, C₁₋₆ alkylsulfonyl, C₁₋₆ alkylthio, and C₁₋₆ alkylsulfinyl.

One embodiment of the present invention relates to the aforementioned compound, or the pharmaceutically acceptable salt, the stereoisomer or the tautomer thereof, wherein Y is selected from

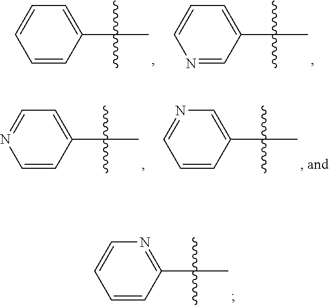 [SMILES: *c1ccccc1 ; *c1cccnc1 ; *c1ccncc1 ; *c1cccnc1 ; *c1ccccn1]

Y is substituted with 1-3 substituents selected from halogen, cyano, hydroxyl, amino, C₁₋₆ alkyl, C₁₋₆ alkoxy, hydroxyl-substituted C₁₋₆ alkyl, haloC₁₋₆ alkyl, aminocarbonyl, C₁₋₆ alkylamino, C₁₋₆ alkylcarbonylamino, C₁₋₆ alkylaminocarbonyl, 3-7 membered heterocyclyl, 3-7 membered cycloalkyl, 5-7 membered heteroaryl, C₁₋₆ alkylsulfonyl, C₁₋₆ alkylthio, and C₁₋₆ alkylsulfinyl.

In some embodiments, the present invention relates to the aforementioned compound, or the pharmaceutically acceptable salt, the stereoisomer or the tautomer thereof,
wherein,
Y is selected from naphthyl, 8-14 membered fused heteroaryl, 6-12 membered fused heterocyclyl, and 6-12 membered fused cycloalkyl; Y is optionally substituted with 1-3 substituents selected from halogen, cyano, amino, hydroxyl, carbonyl, C₁₋₆ alkyl, haloC₁₋₆ alkyl, and C₁₋₆ alkoxy;
with the proviso that: NH N.
when ring A is absent, and R₃ is —NH—R₅, R₅ is not

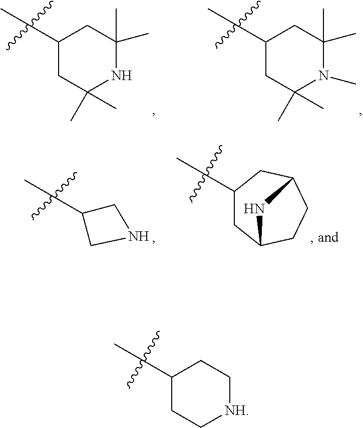 [SMILES: *C1CC(C)(C)NC(C)(C)C1 ; *C1CC(C)(C)N(C)C(C)(C)C1 ; *C1CNC1 ; *C1C[C@H]2CC[C@@H](C1)N2 ; *C1CCNCC1]

In some embodiments, R₃ is selected from —(C₁₋₆ alkylene)₀₋₂-NR₄R₅, —(C₁₋₆ alkylene)₀₋₂-NR₄—COR₅, —(C₁₋₆ alkylene)₀₋₂-CO—NR₄-R₅, and —(C₁₋₆ alkylene)₀₋₂-O—R₅; R₄ is selected from hydrogen and C₁₋₆ alkyl; R₅ is selected from 3-7 membered heterocyclyl, 3-7 membered cycloalkyl, aryl, and 5-7 membered heteroaryl.

In some embodiments, R₄ is hydrogen.

In some embodiments, R₄ is C₁₋₆ alkyl.

In some embodiments, R₄ is selected from methyl and ethyl.

In some embodiments, R₃ is selected from —NR₄R₅, —NH—COR₅, and —O—R₅; R₄ is selected from hydrogen and C₁₋₃ alkyl; R₅ is selected from 3-7 membered cycloalkyl and 3-7 membered heterocyclyl.

In some embodiments, R₃ is —NHR₅, and R₅ is selected from 4-6 membered cycloalkyl and 4-6 membered heterocyclyl.

In some embodiments, R₅ is selected from 4-6 membered cycloalkyl, and 4-6 membered heterocyclyl containing 1 heteroatom selected from O, N, and S.

In some embodiments, R₅ is optionally substituted with 1-4 substituents selected from halogen, cyano, amino, hydroxyl, C₁₋₆ alkyl, haloC₁₋₆ alkyl, C₁₋₆ alkoxy, 3-7 membered heterocyclyl, 3-7 membered cycloalkyl, aryl, 5-7 membered heteroaryl, C₂₋₆ alkenylcarbonyl, sulfonyl, C₁₋₆ alkylcarbonyl, and carboxyl; when R₅ is substituted, the substituents on R₅, which are C₁₋₆ alkyl, haloC₁₋₆ alkyl, C₁₋₆ alkoxy, 3-7 membered heterocyclyl, 3-7 membered cycloalkyl, aryl, 5-7 membered heteroaryl, and sulfonyl, are optionally substituted with 1-3 substituents selected from halogen, cyano, amino, hydroxyl, carbonyl, C₁₋₆ alkyl, and 3-6 membered cycloalkyl.

In some embodiments, R₅ is optionally substituted with 1-2 substituents selected from C₁₋₆ alkyl, hydroxyl, hydroxyl-substituted C₁₋₆ alkyl, 3-7 membered cycloalkyl-substituted C₁₋₆ alkyl, hydroxyl, halogen, C₂₋₆ alkenylcarbonyl, C₁₋₆ alkylsulfonyl, 3-7 membered cycloalkylsulfonyl, aminosulfonyl, 3-7 membered heterocyclyl, 3-7 membered cycloalkyl, carboxyl, and C₁₋₆ alkylcarbonyl.

In some embodiments, the present invention relates to the aforementioned compound, or the pharmaceutically acceptable salt, the stereoisomer or the tautomer thereof, wherein R₃ is selected from —NR₄R₅, —NH—COR₅, and —O—R₅; R₄ is selected from hydrogen and C₁₋₃ alkyl; R₅ is selected from 3-7 membered cycloalkyl and 3-7 membered heterocyclyl, and R₅ is optionally substituted with 1-2 substituents selected from C₁₋₆ alkyl, hydroxyl, hydroxyl-substituted C₁₋₆ alkyl, 3-7 membered cycloalkyl-substituted C₁₋₆ alkyl, hydroxyl, halogen, C₂₋₆ alkenylcarbonyl, C₁₋₆ alkylsulfonyl, 3-7 membered cycloalkylsulfonyl, aminosulfonyl, 3-7 membered heterocyclyl, 3-7 membered cycloalkyl, carboxyl, and C₁₋₆ alkylcarbonyl.

In some embodiments, R₅ is selected from

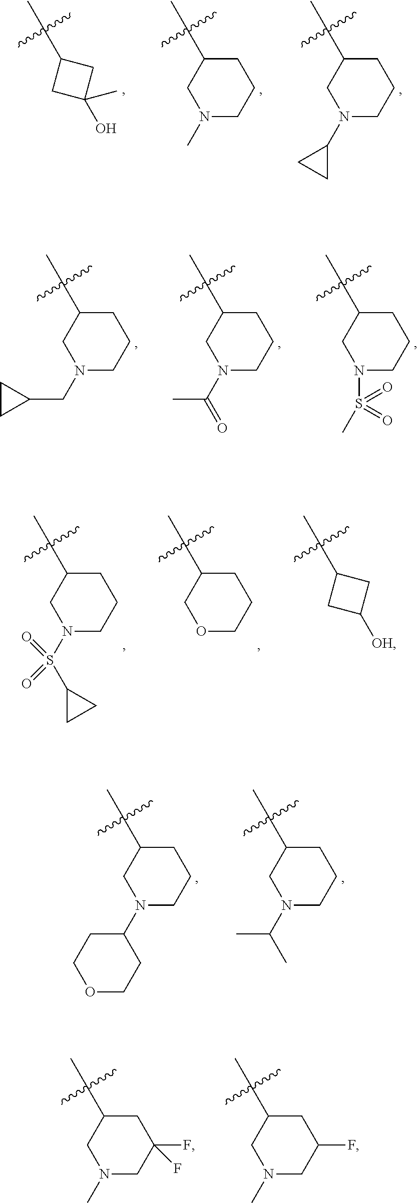

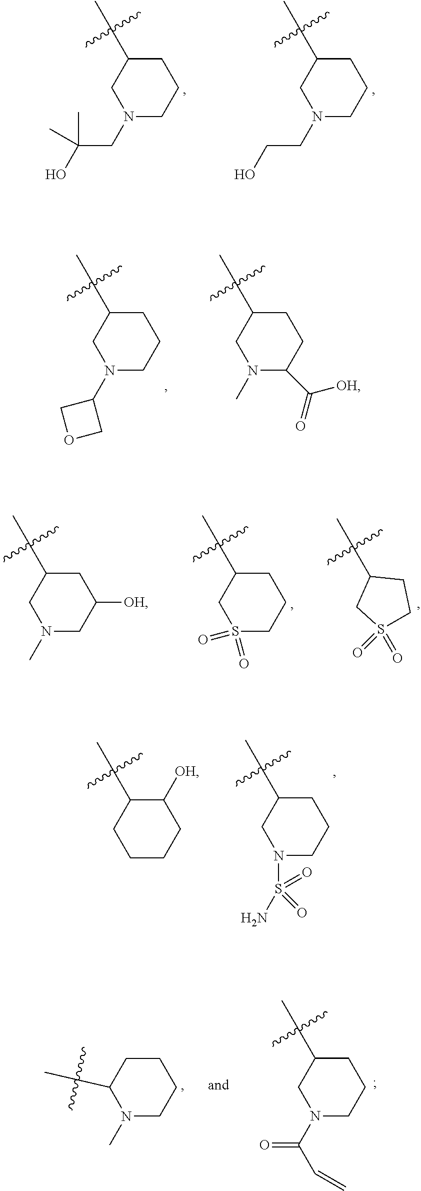

preferably, R₅ is further unsubstituted.

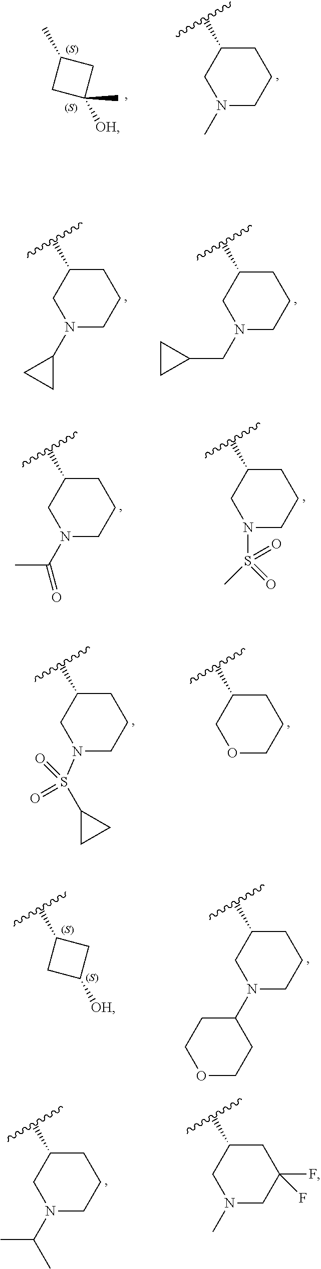

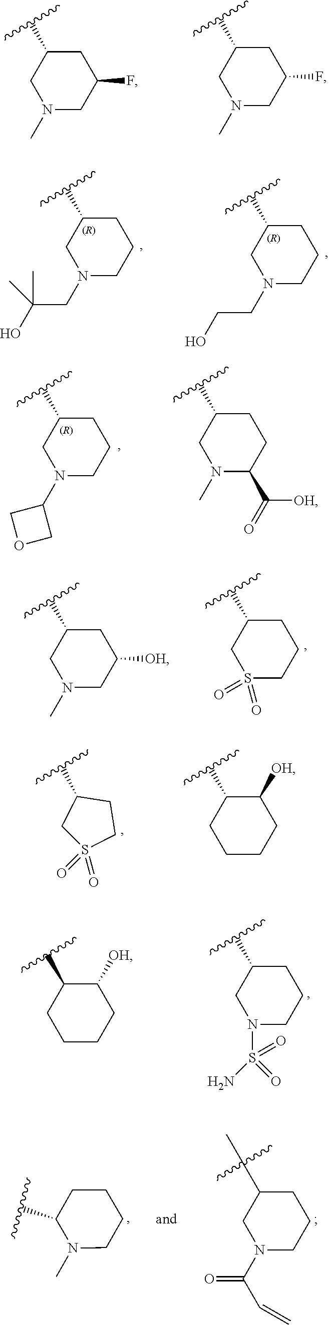

preferably, R₅ is further unsubstituted.

In some embodiments, the present invention relates to the aforementioned compound, or the pharmaceutically acceptable salt, the stereoisomer or the tautomer thereof, wherein R₃ is —NH—R₅, and R₅ is 3-7 membered heterocyclyl substituted with 1-2 substituents selected from C₁₋₆ alkyl; R₅ is preferably 5-6 membered heterocyclyl substituted with 1-2 substituents selected from C₁₋₆ alkyl.

In some embodiments, the present invention relates to the aforementioned compound, or the pharmaceutically acceptable salt, the stereoisomer or the tautomer thereof, wherein ring A is selected from phenyl and 5-7 membered heteroaryl; ring A is optionally substituted with 1-4 substituents selected from hydroxyl, amino, carboxyl, cyano, nitro, halogen, C₁₋₆ alkyl, —NH—C₁₋₆ alkyl, haloC₁₋₆ alkyl, C₁₋₆ alkoxy, haloC₁₋₆ alkoxy, 3-7 membered heterocyclyl, and 3-7 membered cycloalkyl;
Y is selected from phenyl, 5-7 membered heteroaryl, 3-8 membered heterocyclyl, and 3-7 membered cycloalkyl; Y is optionally substituted with 1-3 substituents selected from halogen, cyano, amino, hydroxyl, carbonyl, C₁₋₆ alkyl, haloC₁₋₆ alkyl, C₁₋₆ alkoxy, 3-7 membered heterocyclyl, 3-7 membered cycloalkyl, C₁₋₆ alkylamino, C₁₋₆ alkylcarbonylamino, C₁₋₆ alkylsulfonyl, and aminocarbonyl;
R₃ is —NH—R₅, and R₅ is 3-7 membered heterocyclyl substituted with 1-2 substituents selected from C₁₋₆ alkyl; R₅ is preferably 5-6 membered heterocyclyl substituted with 1-2 substituents selected from C₁₋₆ alkyl.

In some embodiments, Y is optionally substituted with a substituent of C₁₋₆ alkylaminocarbonyl or sulfonyl.

In some embodiments, when R₅ is substituted, the substituents on R₅, which are C₁₋₆ alkyl, haloC₁₋₆ alkyl, C₁₋₆ alkoxy, 3-7 membered heterocyclyl, 3-7 membered cycloalkyl, and sulfonyl, are optionally substituted with 1-3 substituents selected from halogen, cyano, amino, hydroxyl, carbonyl, and C₁₋₆ alkyl.

In some embodiments, the present invention relates to the aforementioned compound, or the pharmaceutically acceptable salt, the stereoisomer or the tautomer thereof, wherein R₁ and R₂, together with the carbon atom to which they are attached, form a 5-8 membered ring A, wherein the 5-8 membered ring A is selected from 5-8 membered cycloalkyl, 5-8 membered cycloalkenyl, 5-8 membered heterocyclyl, phenyl, and 5-6 membered heteroaryl; ring A is optionally substituted with 1-2 substituents selected from cyano, halogen, C₁₋₆ alkyl, —NH—C₁₋₆ alkyl, haloC₁₋₆ alkyl, C₁₋₆ alkoxy, 3-7 membered cycloalkyl, 5-7 membered heteroaryl, C₁₋₆ alkylsulfonyl, and —N(C₁₋₆ alkyl)₂;
Y is selected from phenyl and 5-7 membered heteroaryl; Y is substituted with 1-3 substituents selected from halogen, cyano, hydroxyl, amino, C₁₋₆ alkyl, C₁₋₆ alkoxy, hydroxyl-substituted C₁₋₆ alkyl, haloC₁₋₆ alkyl, aminocarbonyl, C₁₋₆ alkylamino, C₁₋₆ alkylcarbonylamino, C₁₋₆ alkylaminocarbonyl, 3-7 membered heterocyclyl, 3-7 membered cycloalkyl, 5-7 membered heteroaryl, C₁₋₆ alkylsulfonyl, C₁₋₆ alkylthio, and C₁₋₆ alkylsulfinyl;
R₃ is selected from —NR₄R₅, —NH—COR₅, and —O—R₅; R₄ is selected from hydrogen and C₁₋₃ alkyl; R₅ is selected from 3-7 membered cycloalkyl and 3-7 membered heterocyclyl, and R₅ is optionally substituted with 1-2 substituents selected from C₁₋₆ alkyl, hydroxyl or 3-7 membered cycloalkyl-substituted C₁₋₆ alkyl, hydroxyl, halogen, C₂₋₆ alkenylcarbonyl, C₁₋₆ alkylsulfonyl, 3-7 membered cycloalkylsulfonyl, aminosulfonyl, 3-7 membered heterocyclyl, 3-7 membered cycloalkyl, carboxyl, and C₁₋₆ alkylcarbonyl.

On the basis of any of the aforementioned embodiments or technical solutions, when Y is substituted with chloro, R₅ is 6 membered heterocyclyl.

On the basis of any of the aforementioned embodiments or technical solutions, Y is substituted with 1-3 substituents selected from halogen, cyano, amino, hydroxyl, carbonyl, C₁₋₆ alkyl, haloC₁₋₆ alkyl, C₁₋₆ alkoxy, 3-7 membered heterocyclyl, 3-7 membered cycloalkyl, aryl, 5-7 membered heteroaryl, C₁₋₆ alkylamino, C₁₋₆ alkylcarbonylamino, C₁₋₆ alkylsulfonyl, aminocarbonyl, C₁₋₆ alkylaminocarbonyl, sulfonyl, C₁₋₆ alkylthio, and C₁₋₆ alkylsulfinyl; when Y is substituted, the substituents on Y, which are C₁₋₆ alkyl, haloC₁₋₆ alkyl, C₁₋₆ alkoxy, 3-7 membered heterocyclyl, 3-7 membered cycloalkyl, aryl, 5-7 membered heteroaryl, and sulfonyl, are optionally substituted with 1-3 substituents selected from halogen, cyano, amino, hydroxyl, carbonyl, C₁₋₆ alkyl, and 3-6 membered cycloalkyl.

On the basis of any of the aforementioned embodiments or technical solutions,
when Y is hydroxyl-substituted phenyl, R₁ and R₂ are not hydrogen, methyl, cyano, and trifluoromethyl;
when ring A is absent, and R₃ is —NH—R₅, R₅ is not

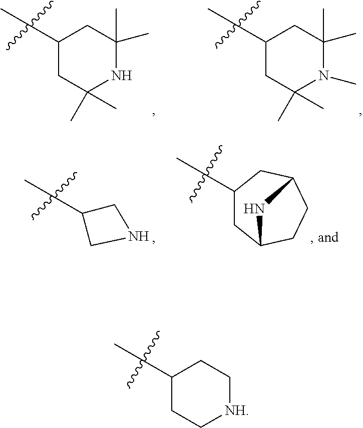 [SMILES: *C1CC(C)(C)NC(C)(C)C1 ; *C1CC(C)(C)N(C)C(C)(C)C1 ; *C1CNC1 ; *C1C[C@H]2CC[C@@H](C1)N2 ; *C1CCNCC1]

In some embodiments, the pharmaceutical composition may comprise one or more pharmaceutically acceptable carriers, and may be administered to a patient or subject in need of such treatment by oral, parenteral, rectal, or transpulmonary administration, and the like. For oral administration, the pharmaceutical composition can be prepared into a conventional solid formulation, such as tablets, capsules, pills and granules; or can be prepared into an oral liquid formulation, such as oral solutions, oral suspensions and syrups. In the preparation of an oral formulation, an appropriate filler, binder, disintegrant, lubricant, and the like may be added. For parenteral administration, the pharmaceutical composition can be formulated into an injection, including a solution injection, a sterile powder for injection, and a concentrated solution for injection. The injection can be produced by a conventional method existing in the pharmaceutical field, and during the preparation process, no additive may be added, or an appropriate additive may be added according to the properties of the medicament. For rectal administration, the pharmaceutical composition can be formulated into a suppository and the like. For transpulmonary administration, the pharmaceutical composition can be formulated into an inhalant, spray, or the like.

### Compound Preparation

The compound disclosed herein can be prepared by a variety of methods including standard chemical methods. Unless otherwise stated, any variable defined above will continue to have the meaning defined above. Exemplary general synthesis methods are elaborated in the following schemes, and can be easily modified to prepare other compounds disclosed herein. In the following detailed description, methods for synthesizing specific compounds according to the present invention are described.

The compound of formula (II) may be prepared by coupling a compound of formula (a) with a compound of formula (III);

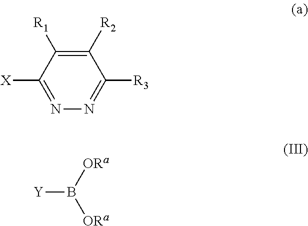

wherein,
X is halogen (e.g., iodine, bromine, or chlorine); R^{a} is selected from H and C₁₋₆ alkyl, or R^{a}, together with B and O atoms, forms a 5-7 membered heterocyclic ring; R₁, R₂ and Y are as defined above.

Provided below is a method for preparing a compound of formula (II) by reacting a compound of formula (a) with a compound of formula (III):

For example, the compound of formula (a) and the compound of formula (III) are added to a suitable solvent (such as 1,4-dioxane and water), a suitable catalyst (such as [1,1′-bis(diphenylphosphino)ferrocene]dichloropalladium(II)) and a suitable base (such as sodium bicarbonate) are added, and the resulting mixture is heated and stirred at a suitable temperature (such as 90° C. to 110° C.) for a suitable period of time (such as 1 to 20 hours) under the protection of an inert gas (such as nitrogen). After the completion of reaction, the reaction solution is added to an appropriate amount of water, followed by extraction with a suitable extractant (such as ethyl acetate). After concentration under reduced pressure, the compound of formula (II) is separated out by using a suitable purification method (such as silica gel column chromatography or preparative thin-layer chromatography).

The compound of formula (a) may be prepared by coupling a compound of formula (b) with a compound of formula (IV);

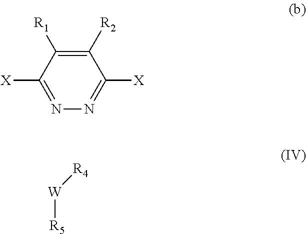

wherein X is halogen (e.g., iodine, bromine, or chlorine); W is OH or NH, and R₁, R₂, R₄ and R₅ are as defined above.

The method for preparing the compound of formula (a) by reacting the compound of formula (b) with the compound of formula (IV) is as follows:

For example, the compound of formula (b) and the compound of formula (IV) are added to a suitable solvent (e.g., 1,4-dioxane), a suitable catalyst (e.g., tris(dibenzylideneacetone)dipalladium(0)), a suitable ligand (e.g., 1,1′-binaphthyl-2,2′-bisdiphenylphosphine), and a suitable base (e.g., cesium carbonate) are added, and the resulting mixture is heated and stirred at a suitable temperature (e.g., 90° C. to 110° C.) for a suitable period of time (e.g., 1 to 12 hours). After the completion of reaction, the reaction solution is poured into an appropriate amount of water, followed by extraction with a suitable extractant (such as ethyl acetate). After concentration under reduced pressure, the compound of formula (a) is separated out by using a suitable purification method (such as silica gel column chromatography or preparative thin-layer chromatography).

In some embodiments, the compound of formula (a) may be prepared by reacting the compound of formula (IV) with the compound of formula (b) under a heating condition.

For example, the compound of formula (b) and the compound of formula (IV) are added to a suitable solvent (e.g., N,N-dimethylacetamide), and the resulting mixture was stirred at a suitable temperature (e.g., 120° C.) for a suitable period of time (e.g., 16 to 20 hours). After the completion of reaction, the reaction solution is poured into an appropriate amount of water, followed by extraction with a suitable extractant (such as ethyl acetate). After concentration under reduced pressure, the compound of formula (a) is separated out by using a suitable purification method (such as silica gel column chromatography or preparative thin-layer chromatography).

In some embodiments, the compound of formula (II) may be prepared by coupling the compound of formula (i) with the compound of formula (IV);

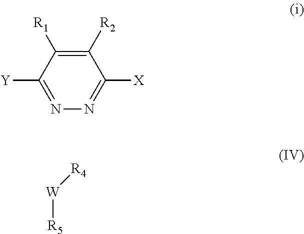

wherein,
X, R₁, R₂, Y, W, R₄, and R₅ are as defined above.

The method for preparing the compound of formula (II) by reacting the compound of formula (i) with the compound of formula (IV) is as follows:

For example, the compound of formula (i) and the compound of formula (IV) are added to a suitable solvent (e.g., 1,4-dioxane), a suitable catalyst (e.g., tris(dibenzylideneacetone)dipalladium(0)), a suitable ligand (e.g., 1,1′-binaphthyl-2,2′-bisdiphenylphosphine), and a suitable base (e.g., cesium carbonate) are added, and the resulting mixture is heated and stirred at a suitable temperature (e.g., 90° C. to 110° C.) for a suitable period of time (e.g., 1 to 12 hours). After the completion of reaction, the reaction solution is poured into an appropriate amount of water, followed by extraction with a suitable extractant (such as ethyl acetate). After concentration under reduced pressure, the compound of formula (II) is separated out by using a suitable purification method (such as silica gel column chromatography or preparative thin-layer chromatography).

In some embodiments, the compound of formula (II) may be prepared by reacting the compound of formula (i) with the compound of formula (IV) under a heating condition.

For example, the compound of formula (i) and the compound of formula (IV) are added to a suitable solvent (e.g., N,N-dimethylacetamide), and the resulting mixture was stirred at a suitable temperature (e.g., 120° C.) for a suitable period of time (e.g., 16 to 20 hours). After the completion of reaction, the reaction solution is poured into an appropriate amount of water, followed by extraction with a suitable extractant (such as ethyl acetate). After concentration under reduced pressure, the compound of formula (II) is separated out by using a suitable purification method (such as silica gel column chromatography or preparative thin-layer chromatography).

The compound of formula (i) may be prepared by coupling a compound of formula (b) with a compound of formula (III);

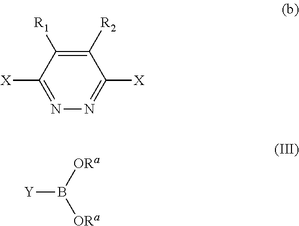

wherein,
X, R₁, R₂, Y, and R^{a} are as defined above.

The method for preparing the compound of formula (i) by reacting the compound of formula (b) with the compound of formula (III) is as follows:

For example, the compound of formula (b) and the compound of formula (III) are added to a suitable solvent (such as 1,4-dioxane and water), a suitable catalyst (such as [1,1′-bis(diphenylphosphino)ferrocene]dichloropalladium(II)) and a suitable base (such as sodium bicarbonate) are added, and the resulting mixture is heated and stirred at a suitable temperature (such as 90° C. to 110° C.) for a suitable period of time (such as 1 to 20 hours) under the protection of an inert gas (such as nitrogen). After the completion of reaction, the reaction solution is added to an appropriate amount of water, followed by extraction with a suitable extractant (such as ethyl acetate). After concentration under reduced pressure, the compound of formula (i) is separated out by using a suitable purification method (such as silica gel column chromatography or preparative thin-layer chromatography).

In some embodiments, the compound of formula (b) may be prepared by halogenating a compound of formula (c);

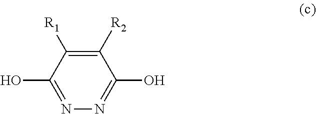

R₁ and R₂ are as defined above.

The method for preparing the compound of formula (b) from the compound of formula (c) is as follows:

For example, the compound of formula (c) is added to a suitable solvent (e.g., acetonitrile), a halogenating agent (e.g., phosphorus oxychloride) is added, and the resulting mixture is heated and stirred at a suitable temperature (e.g., 25° C. to 90° C.) for a suitable period of time (e.g., 1 to 12 hours). After the completion of reaction, the reaction solution is poured into an appropriate amount of water, followed by extraction with a suitable extractant (such as ethyl acetate). After concentration under reduced pressure, the compound of formula (b) is separated out by using a suitable purification method (such as silica gel column chromatography or preparative thin-layer chromatography).

In some embodiments, the compound of formula (c) may be prepared from a compound of formula (d);

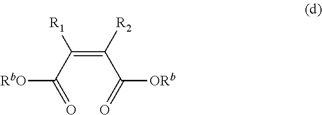

wherein,
R^{b} is C₁₋₆ alkyl; R₁ and R₂ are as defined above.

The method for preparing the compound of formula (c) from the compound of formula (d) is as follows:

For example, the compound of formula (d) (or an anhydride compound of ring A) is added to a suitable solvent (e.g., ethanol), hydrazine hydrate is added, and the resulting mixture is heated and stirred at a suitable temperature (e.g., 25° C. to 80° C.) for a suitable period of time (e.g., 0.5 to 5 hours). After the completion of reaction, the reaction solution is poured into an appropriate amount of water, followed by extraction with a suitable extractant (such as ethyl acetate). After concentration under reduced pressure, the compound of formula (c) is separated out by using a suitable purification method (such as silica gel column chromatography or preparative thin-layer chromatography).

In some embodiments, the compound of formula (i) may be prepared by the following reaction formula;

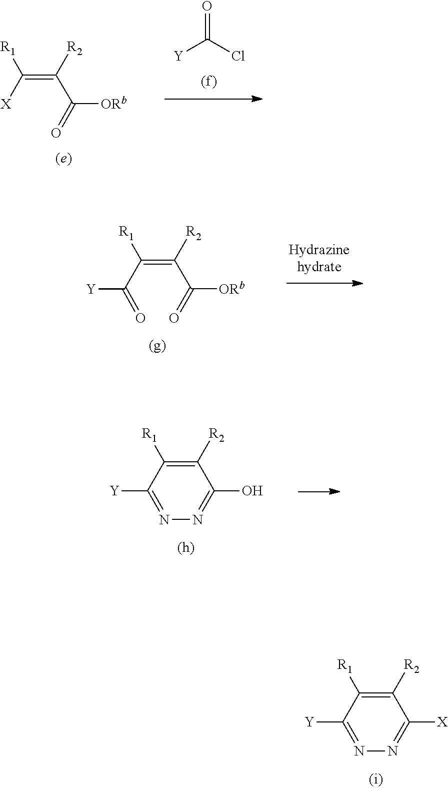

wherein X, R₁, R₂, Y, and R^{b} are as defined above.

The specific method for preparing the compound of formula (i) is as follows:

For example, a compound of formula (e) is added to a suitable solvent (e.g., tetrahydrofuran), the resulting mixture is stirred at a suitable temperature (e.g., −70° C. to 0° C.) for a suitable period of time (e.g., 0.5 to 1 hour), and then a solution of a compound of formula (f) (e.g., tetrahydrofuran) is added at a suitable temperature (e.g., −70° C. to 0° C.) for a suitable period of time (e.g., 2 to 5 hours). After the completion of reaction, the reaction solution is poured into an appropriate amount of water, followed by extraction with a suitable extractant (such as ethyl acetate). After concentration under reduced pressure, the compound of formula (g) is separated out by using a suitable purification method (such as silica gel column chromatography or preparative thin-layer chromatography).

The compound of formula (g) is added to a suitable solvent (such as ethanol), hydrazine hydrate is added, and the resulting mixture is heated and stirred at a suitable temperature (such as 25° C. to 80° C.) for a suitable period of time (such as 0.5 to 5 hours). After the completion of reaction, the reaction solution is poured into an appropriate amount of water, followed by extraction with a suitable extractant (such as ethyl acetate). After concentration under reduced pressure, the compound of formula (h) is separated out by using a suitable purification method (such as silica gel column chromatography or preparative thin-layer chromatography).

The compound of formula (h) is added to a suitable solvent (e.g., acetonitrile), a halogenating agent (e.g., phosphorus oxychloride) is added, and the resulting mixture is heated and stirred at a suitable temperature (e.g., 25° C. to 90° C.) for a suitable period of time (e.g., 1 to 12 hours). After the completion of reaction, the reaction solution is poured into an appropriate amount of water, followed by extraction with a suitable extractant (such as ethyl acetate). After concentration under reduced pressure, the compound of formula (i) is separated out by using a suitable purification method (such as silica gel column chromatography or preparative thin-layer chromatography).

In some embodiments, the compound of formula (h) may be prepared by the following reaction formula;

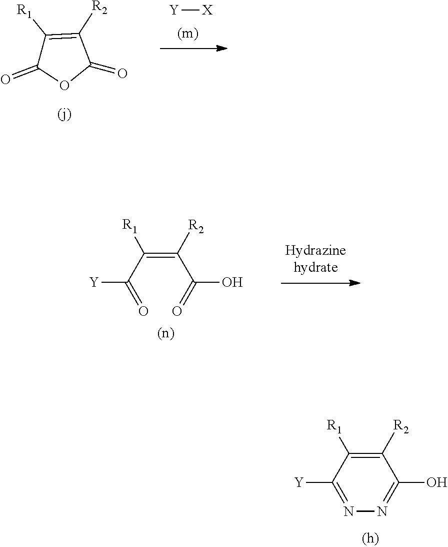

wherein,
X, R₁, R₂, and Y are as defined above.

For example, the compound of formula (m) is added to a suitable solvent (e.g., tetrahydrofuran), an organometallic reagent (e.g., isopropyl magnesium chloride) is added at a suitable temperature (e.g., −60° C. to 0° C.), and stirred for a suitable period of time (e.g., 0.5 to 2 hours), and then the solution is added to a solution of a compound of formula (j) (e.g., tetrahydrofuran) at a suitable temperature (e.g., −70° C. to 0° C.) and stirred for a suitable period of time (e.g., 2 to 5 hours). After the completion of reaction, the reaction solution is poured into an appropriate amount of water to adjust the pH, followed by extraction with a suitable extractant (such as ethyl acetate). After concentration under reduced pressure, a compound of formula (n) is separated out by using a suitable purification method (such as silica gel column chromatography or preparative thin-layer chromatography).

The compound of formula (n) is added to a suitable solvent (such as ethanol), hydrazine hydrate is added, and the resulting mixture is heated and stirred at a suitable temperature (such as 25° C. to 80° C.) for a suitable period of time (such as 0.5 to 5 hours). After the completion of reaction, the reaction solution is poured into an appropriate amount of water, followed by extraction with a suitable extractant (such as ethyl acetate). After concentration under reduced pressure, the compound of formula (h) is separated out by using a suitable purification method (such as silica gel column chromatography or preparative thin-layer chromatography).

The above preparation methods and the order of the respective steps in the methods are merely exemplary, and those skilled in the art can prepare the compounds of the present invention with reference to the above methods and can adjust the order of the steps as the case may be. In addition, the compounds whose sources are not described in the above preparation methods are generally commercially available, or may be synthesized from commercially available compounds by a method known to those skilled in the art.

### Beneficial Effects of Present Invention

The research finds that the compound provided in the present invention, or the pharmaceutically acceptable salt, the stereoisomer or the tautomer thereof has good inhibitory activity against the NLRP3 inflammasome, so that the compound of the present invention can be used for preventing and/or treating NLRP3 inflammasome-associated diseases.

### DETAILED DESCRIPTION

In order to make the objective, technical schemes, and advantages of the present invention more apparent, the present invention is further described in detail below. It should be apparent that the examples described herein are only some examples of the present invention, but not all examples. Based on the examples of the present invention, all other examples obtained by those of ordinary skill in the art without creative work shall fall within the protection scope of the present invention.

The abbreviations and English expressions used in the present invention have the following meanings:

“THF” refers to tetrahydrofuran; “DMF” refers to N,N-dimethylformamide; “MeOH” refers to methanol; “EA” refers to ethyl acetate; “DCM” refers to dichloromethane; “DMA” refers to N,N-dimethylacetamide; “MTBE” refers to methyl tert-butyl ether; “EtOH” refers to ethanol; “DMAC” refers to dimethylacetamide; “PE” refers to petroleum ether; “n-BuLi” refers to n-butyllithium;

“FBS” refers to fetal bovine serum; “PBS” refers to phosphate-buffered saline solution; “PMA” refers to phorbol ester; “LPS” refers to lipopolysaccharide; “Nigericin” refers to nigericin sodium salt.

### Example 1: Synthesis of (R)-5-methyl-2-(4-((1-methylpiperidin-3-yl)amino)phthalazin-1-yl)phenol (Compound 1)

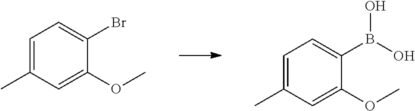

### Step 1: Synthesis of (2-methoxy-4-methylphenyl)boronic acid

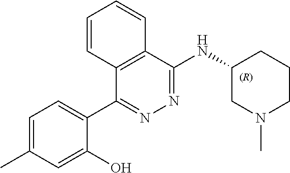

1-Bromo-2-methoxy-4-methylbenzene (8.0 g, 39.78 mmol, 1.0 eq.) was added to THE (60.0 mL), and the mixture was cooled to −70° C., followed by dropwise and slow addition of a solution of 2.5 M n-butyllithium in n-hexane (23.8 mL, 59.67 mmol, 1.5 eq.). The reaction solution was stirred at −70° C. for 2 h, triisopropyl borate (9.72 g, 51.72 mmol, 1.3 eq.) was added dropwise and slowly, and the resulting mixture was gradually warmed to room temperature and stirred for 4 h. After the reaction was completed as monitored by TLC, a 2 mol/L aqueous HCl solution (100.0 mL) was added to the system with stirring for 1 h, and EA (200.0 mL) was added, followed by liquid separation. The organic phase was dried over anhydrous sodium sulfate and filtered, the filtrate was concentrated under reduced pressure, and slurried with petroleum ether (50.0 mL) for 1 h, and the residue was filtered under vacuum to give the product (5.8 g, yield: 87.8%).

### Step 2: Synthesis of 1-chloro-4-(2-methoxy-4-methylphenyl)phthalazine

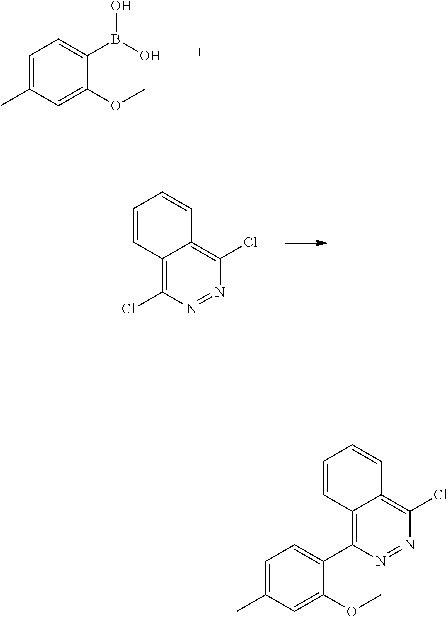

(2-Methoxy-4-methylphenyl)boronic acid (5.19 g, 31.26 mmol, 1.0 eq.) was added to 1,4-dioxane (60.0 mL), 1,4-dichlorophthalazine (6.84 g, 34.39 mmol, 1.1 eq.) was added, followed by the addition of sodium bicarbonate (5.25 g, 62.52 mmol, 2.0 eq.), water (15.0 mL) and Pd(dppf)Cl₂ (1.14 g, 1.56 mmol, 0.05 eq.), and the mixture was reacted at 90° C. for 5 h in nitrogen atmosphere. After the reaction was completed as monitored by TLC, EA (200.0 mL) and water (200 mL) were added to the system, followed by liquid separation. The organic phase was dried over anhydrous sodium sulfate and filtered, the filtrate was concentrated under reduced pressure, and the crude product was purified by silica gel column chromatography (100-200 mesh silica gel, petroleum ether:ethyl acetate=10:1 to 4:1) to give the product (4.4 g, yield: 49.4%).

Step 3: Synthesis of 2-(4-bromophthalazin-1-yl)-5-methylphenol

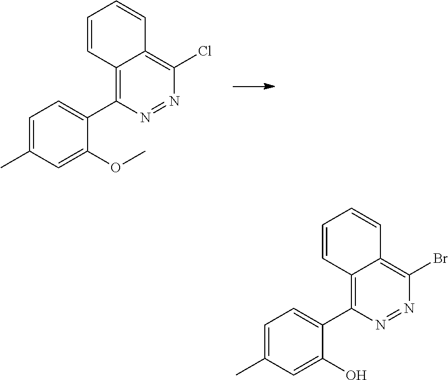 [SMILES: COc1cc(C)ccc1-c1nnc(Cl)c2ccccc12 ; Cc1ccc(-c2nnc(Br)c3ccccc23)c(O)c1]

1-Chloro-4-(2-methoxy-4-methylphenyl)phthalazine (4.0 g, 14.04 mmol, 1.0 eq.) was added to DCM (30.0 mL), the mixture was cooled to −65° C., followed by dropwise and slow addition of boron tribromide (7.0 g, 28.09 mmol, 2.0 eq.), and the mixture was gradually warmed to room temperature and stirred overnight. After the reaction was completed as monitored by TLC, methanol was added to the system at 0° C. to quench the reaction, and after concentration under reduced pressure, DCM and water were added for liquid separation. The organic phase was dried over anhydrous sodium sulfate and filtered, and the filtrate was concentrated under reduced pressure to give the product (2.3 g, yield: 52.2%).

### Step 4: Synthesis of tert-butyl (R)-3-((4-(2-hydroxy-4-methylphenyl) phthalazin-1-yl)amino)piperidine-1-carboxylate

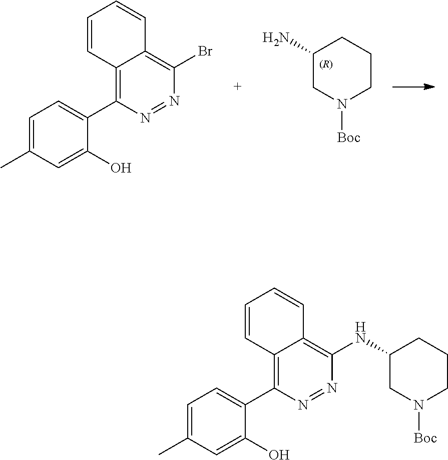

2-(4-Bromophthalazin-1-yl)-5-methylphenol (2.3 g, 7.29 mmol, 1.0 eq.) was added to DMA (10.0 mL) and tert-butyl (R)-3-aminopiperidine-1-carboxylate (2.19 g, 10.94 mmol, 1.5 eq.), and the mixture was stirred at 120° C. overnight. After the reaction was completed as monitored by TLC, EA (200.0 mL) was added to the system, followed by washing with water (100.0 mL×3). The organic phase was dried over anhydrous sodium sulfate and filtered, the filtrate was concentrated under reduced pressure, and the crude product was purified by silica gel column chromatography (100-200 mesh silica gel, petroleum ether:ethyl acetate=100:1 to 90:1) to give the product (1.8 g, yield: 56.7%).

### Step 5: Synthesis of (R)-5-methyl-2-(4-(piperidin-3-ylamino)phthalazin-1-yl)phenol

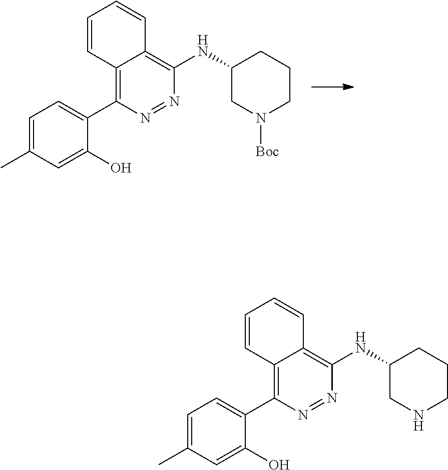 [SMILES: Cc1ccc(-c2nnc(N[C@@H]3CCCN(C(=O)OC(C)(C)C)C3)c3ccccc23)c(O)c1 ; Cc1ccc(-c2nnc(N[C@@H]3CCCNC3)c3ccccc23)c(O)c1]

Tert-butyl (R)-3-((4-(2-hydroxy-4-methylphenyl)phthalazin-1-yl)amino) piperidine-1-carboxylate (750.0 mg, 1.72 mmol, 1.0 eq.) was added to DCM (5.0 mL) and trifluoroacetic acid (5.0 mL), and the mixture was stirred at room temperature for 2 h. After the reaction was completed as monitored by TLC, the system was back-extracted with water (30.0 mL×4), and the aqueous phases were combined, followed by addition of a saturated aqueous sodium carbonate solution to adjust the pH to 8-9, and addition of DCM for extraction for 3 times. The organic phases were combined, dried over anhydrous sodium sulfate, and filtered, and the filtrate was concentrated under reduced pressure to give the product (550.0 mg, yield: 95.4%).

### Step 6: Synthesis of (R)-5-methyl-2-(4-((1-methylpiperidin-3-yl)amino) phthalazin-1-yl)phenol

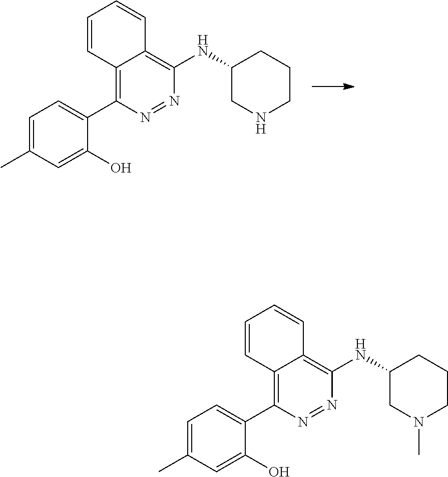 [SMILES: Cc1ccc(-c2nnc(N[C@@H]3CCCNC3)c3ccccc23)c(O)c1 ; Cc1ccc(-c2nnc(N[C@@H]3CCCN(C)C3)c3ccccc23)c(O)c1]

(R)-5-methyl-2-(4-(piperidin-3-ylamino)phthalazin-1-yl)phenol (550.0 mg, 1.64 mmol, 1.0 eq.) was added to methanol (10.0 mL), and an aqueous formaldehyde solution (37%) (160.1 mg, 1.97 mmol, 1.2 eq.) was added. The mixture was stirred at room temperature for 1 h, followed by addition of sodium cyanoborohydride (144.6 mg, 2.30 mmol, 1.4 eq.), and the resulting mixture was reacted at room temperature for 3 h. After the reaction was completed as monitored by TLC, EA (100.0 mL) and a saturated aqueous sodium bicarbonate solution were added to the system, followed by liquid separation. The organic phase was dried over anhydrous sodium sulfate and filtered, the filtrate was concentrated under reduced pressure, and the crude product was purified by preparative thin-layer chromatography (DCM:MeOH=8:1) to give the product (300.0 mg, yield: 52.3%).

¹HNMR (300 MHz, DMSO-d6) δ (ppm): 9.63 (s, 1H), 8.48 (s, 1H), 7.77-7.89 (m, 2H), 7.52-7.54 (m, 1H), 7.41 (s, 1H), 7.15-7.17 (m, 1H), 6.76-6.82 (m, 2H), 4.65 (s, 1H), 3.36-3.47 (m, 2H), 3.12-3.17 (m, 2H), 2.50-2.51 (m, 3H), 2.33 (s, 3H), 1.77-1.99 (m, 4H).

Molecular formula: C₂₁H₂₄N₄O molecular weight: 348.45 LC-MS (Pos, m/z)=349.22 [M+H]⁺.

### Example 2: Synthesis of (R)-5-methyl-2-(8-methyl-4-((1-methylpiperidin-3-yl)amino)phthalazin-1-yl)phenol (Compound 2) and (R)-5-methyl-2-(5-methyl-4-((1-methylpiperidin-3-yl)amino)phthalazin-1-yl)phenol (Compound 5)

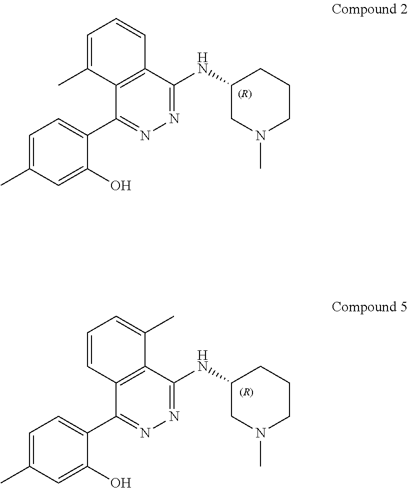

Step 1: Synthesis of 2-(2-methoxy-4-methylbenzoyl)-3-methylbenzoic acid and 2-(2-methoxy-4-methylbenzoyl)-6-methylbenzoic acid

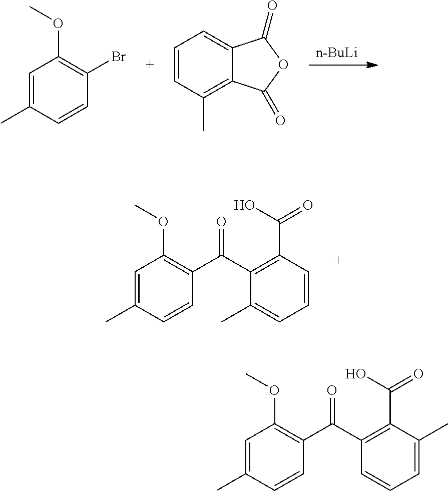

1-Bromo-2-methoxy-4-methylbenzene (6.82 g, 33.91 mmol, 1.1 eq.) was dissolved in THE (50 mL), and the mixture was cooled to −50° C., followed by dropwise and slow addition of a solution of n-butyllithium in n-hexane (2.5 mol/L, 13.6 mL, 33.91 mmol, 1.1 eq.). The reaction solution was stirred at −50° C. for 30 min and then added dropwise and slowly to a solution of 4-methylisobenzofuran-1,3-dione (5 g, 30.83 mmol, 1.0 eq.) in THE (50 mL) at −60° C., and reacted at −60° C. in nitrogen atmosphere. After the completion of dropwise addition, the reaction solution was naturally warmed to room temperature and reacted for 1 h, and then poured into a dilute hydrochloric acid solution (100 mL), followed by extraction with EA (100 mL×2). The organic phase was washed with 0.2 mol/L NaOH solution (100 mL), hydrochloric acid was added to the aqueous phase to adjust the pH to 4, followed by extraction with EA (100 mL×2), and the organic phase was dried and concentrated to give a mixture (7.0 g, yield: 79.9%) of 2-(2-methoxy-4-methylbenzoyl)-3-methylbenzoic acid and 2-(2-methoxy-4-methylbenzoyl)-6-methylbenzoic acid.

### Step 2: Synthesis of methyl 2-(2-methoxy-4-methylbenzoyl)-3-methylbenzoate and methyl 2-(2-methoxy-4-methylbenzoyl)-6-methylbenzoate

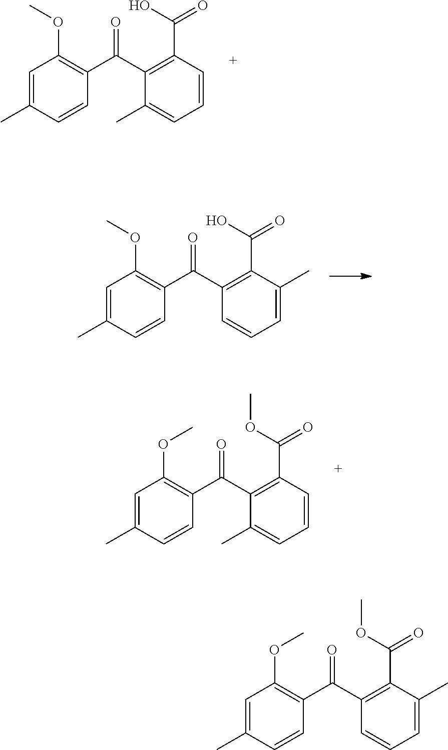

A mixture (7.0 g, 24.62 mmol, 1.0 eq.) of 2-(2-methoxy-4-methylbenzoyl)-3-methylbenzoic acid and 2-(2-methoxy-4-methylbenzoyl)-6-methylbenzoic acid, potassium carbonate (5.1 g, 36.93 mmol, 1.5 eq.) and iodomethane (5.24 g, 36.93 mmol, 1.5 eq.) were dissolved in DMF (100 mL), and the mixture was reacted at room temperature for 1 h. After the reaction was completed as monitored by TLC, the reaction solution was poured into water (100 mL), followed by extraction with methyl tert-butyl ether (50 mL×3). The organic phase was washed with water (30 mL×2), dried, and concentrated to give a mixture (5.0 g, yield: 68.1%) of methyl 2-(2-methoxy-4-methylbenzoyl)-3-methylbenzoate and methyl 2-(2-methoxy-4-methylbenzoyl)-6-methylbenzoate.

### Step 3: Synthesis of 4-(2-methoxy-4-methylphenyl)-5-methylphthalazin-1(2H)-one and 4-(2-methoxy-4-methylphenyl)-8-methylphthalazin-1(2H)-one

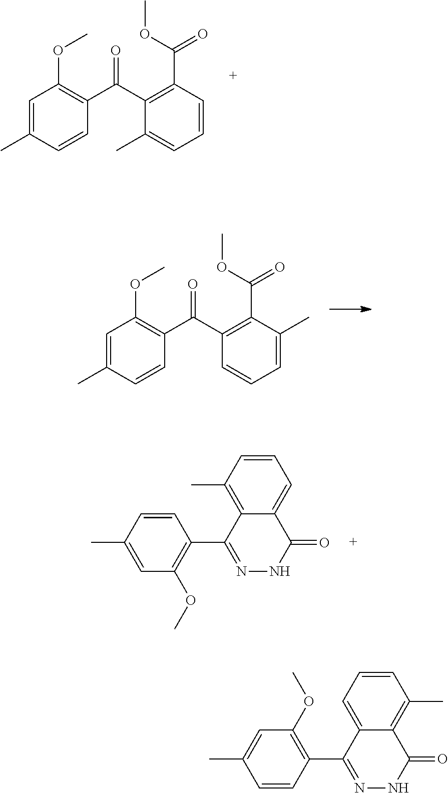

A mixture (5.0 g, 16.76 mmol, 1.0 eq.) of methyl 2-(2-methoxy-4-methylbenzoyl)-3-methylbenzoate and methyl 2-(2-methoxy-4-methylbenzoyl)-6-methylbenzoate, and 85% hydrazine hydrate (1.48 g, 25.14 mmol, 1.5 eq.) were dissolved in ethanol (50 mL), and the mixture was reacted at 80° C. for 9 h. After the reaction was substantially completed as detected by LC-MS, the reaction solution was concentrated under reduced pressure, and the crude product was slurried with EtOH: MTBE=(3:1, 40 mL), followed by filtration under vacuum. The filter cake was dried to give a mixture (4.0 g, yield: 85.1%) of 4-(2-methoxy-4-methylphenyl)-5-methylphthalazin-1(2H)-one and 4-(2-methoxy-4-methylphenyl)-8-methylphthalazin-1(2H)-one.

### Step 4: Synthesis of 1-chloro-4-(2-methoxy-4-methylphenyl)-5-methylphthalazine and 4-chloro-1-(2-methoxy-4-methylphenyl)-5-methylphthalazine

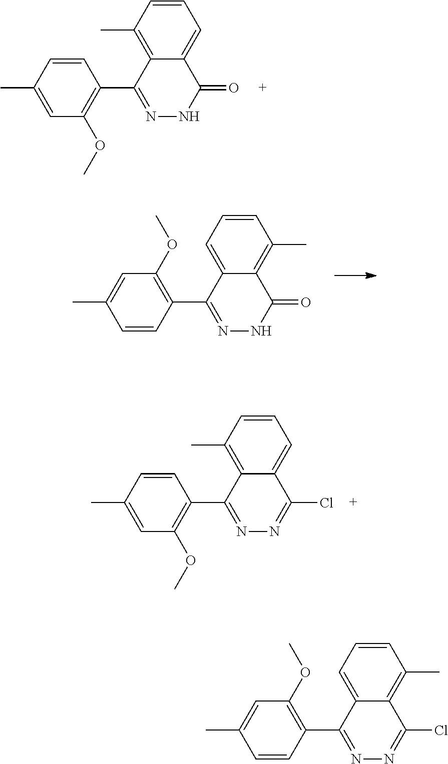

4-(2-Methoxy-4-methylphenyl)-5-methylphthalazin-1(2H)-one, 4-(2-methoxy-4-methylphenyl)-8-methylphthalazin-1(2H)-one (4.0 g, 14.26 mmol, 1.0 eq.) and phosphorus oxychloride (4.37 g, 28.52 mmol, 2.0 eq.) were dissolved in acetonitrile (40 mL), and the mixture was reacted at 90° C. for 13.5 h. After the reaction was completed as monitored by TLC, the reaction solution was concentrated under reduced pressure, and the crude product was dissolved in water (50 mL), followed by addition of sodium carbonate to adjust the pH to 9, and extraction with EA (100 mL×2). The organic phase was dried and concentrated to give a mixture (3.6 g, yield: 84.5%) of 1-chloro-4-(2-methoxy-4-methylphenyl)-5-methylphthalazine and 4-chloro-1-(2-methoxy-4-methylphenyl)-5-methylphthalazine.

### Step 5: Synthesis of tert-butyl (R)-3-((4-(2-methoxy-4-methylphenyl)-5-methylphthalazin-1-yl)amino)piperidine-1-carboxylate and tert-butyl (R)-3-((4-(2-methoxy-4-methylphenyl)-8-methylphthalazin-1-yl)amino)piperidine-1-carboxylate

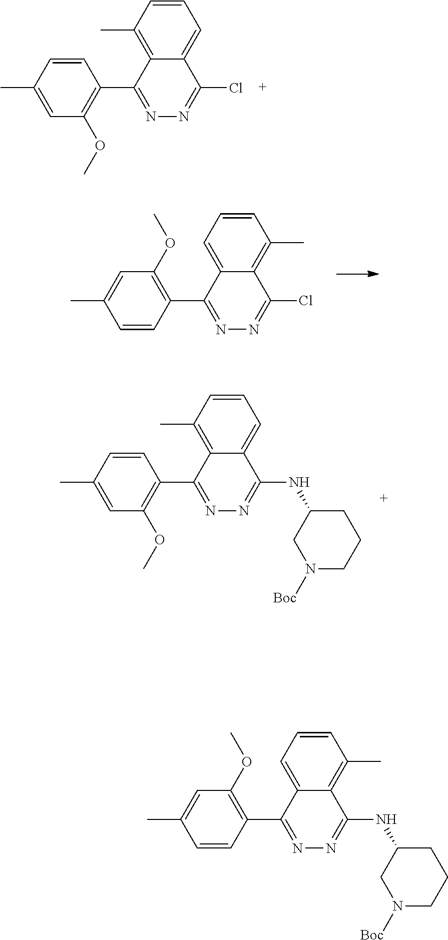

A mixture (3.6 g, 12.05 mmol, 1.0 eq.) of 1-chloro-4-(2-methoxy-4-methylphenyl)-5-methylphthalazine and 4-chloro-1-(2-methoxy-4-methylphenyl)-5-methylphthalazine, and tert-butyl (R)-3-aminopiperidine-1-carboxylate (4.83 g, 24.10 mmol, 2.0 eq.) were dissolved in DMAC (20 mL), and the resulting mixture was reacted at 120° C. for 11 h. After the reaction was completed as monitored by TLC, the reaction solution was poured into water (100 mL), and solids precipitated, followed by filtration under vacuum. The filter cake was dissolved in EA (100 mL), and washed with water (20 mL×3), and the organic phase was dried and concentrated to give a mixture (4.0 g, yield: 71.8%) of tert-butyl (R)-3-((4-(2-methoxy-4-methylphenyl)-5-methylphthalazin-1-yl)amino)piperidine-1-carboxylate and tert-butyl (R)-3-((4-(2-methoxy-4-methylphenyl)-8-methylphthalazin-1-yl)amino)piperidine-1-carboxylate.

### Step 6: Synthesis of (R)-5-methyl-2-(8-methyl-4-(piperidin-3-ylamino) phthalazin-1-yl)phenol and (R)-5-methyl-2-(5-methyl-4-(piperidin-3-ylamino)phthalazin-1-yl)phenol

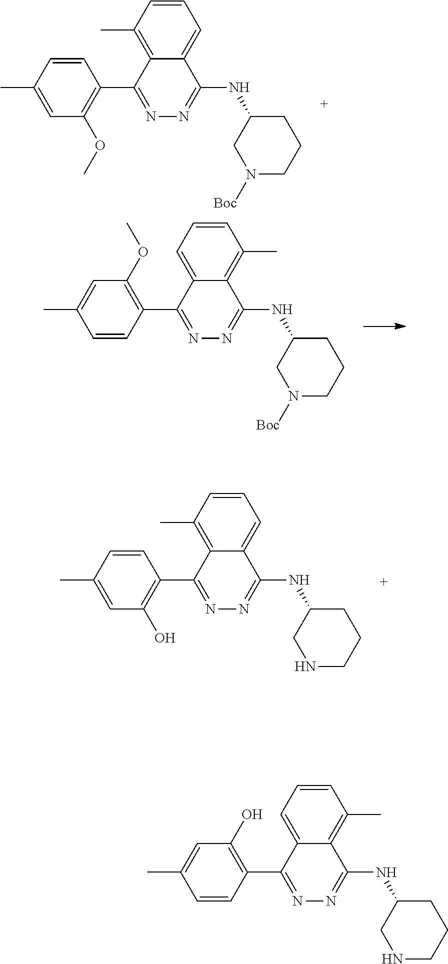

A mixture (4.0 g, 8.64 mmol, 1.0 eq.) of tert-butyl (R)-3-((4-(2-methoxy-4-methylphenyl)-5-methylphthalazin-1-yl)amino)piperidine-1-carboxylate and tert-butyl (R)-3-((4-(2-methoxy-4-methylphenyl)-8-methylphthalazin-1-yl)amino)piperidine-1-carboxylate was dissolved in DCM (20 mL), and the mixture was cooled to −20° C., followed by dropwise addition of a 1 mol/L solution of boron tribromide in dichloromethane (17 mL, 17.28 mmol, 2.0 eq.). After the completion of dropwise addition, the reaction solution was naturally warmed to room temperature and reacted for 1 h, and after the reaction was completed as detected by TLC, the reaction solution was poured into water (20 mL), followed by liquid separation. NaHCO₃ was added to the aqueous phase to adjust the pH to 9, and then extracted with DCM (30 mL×2), and the organic phase was dried and concentrated to give a mixture (2.5 g, yield: 83.3%) of (R)-5-methyl-2-(8-methyl-4-(piperidin-3-ylamino)phthalazin-1-yl)phenol and (R)-5-methyl-2-(5-methyl-4-(piperidin-3-ylamino)phthalazin-1-yl)phenol.

### Step 7: Synthesis of (R)-5-methyl-2-(8-methyl-4-((1-methylpiperidin-3-yl)amino)phthalazin-1-yl)phenol and (R)-5-methyl-2-(5-methyl-4-((1-methylpiperidin-3-yl)amino)phthalazin-1-yl)phenol

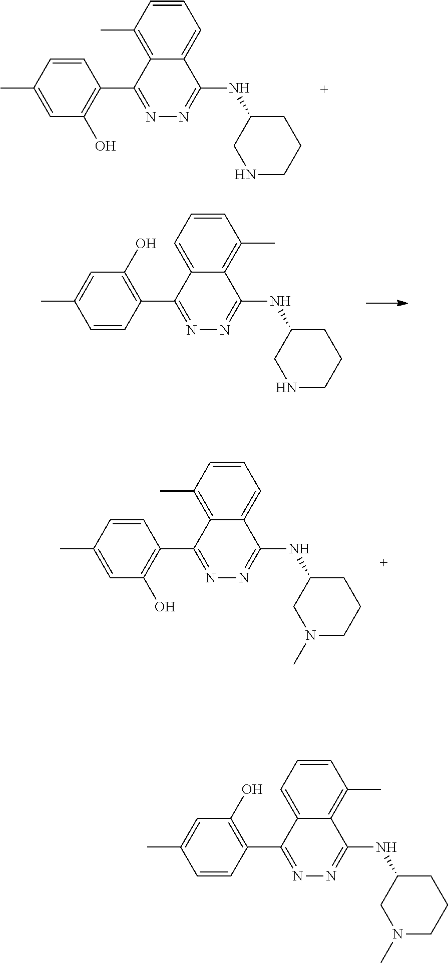 [SMILES: Cc1ccc(-c2nnc(N[C@@H]3CCCNC3)c3cccc(C)c23)c(O)c1 ; Cc1ccc(-c2nnc(N[C@@H]3CCCNC3)c3c(C)cccc23)c(O)c1 ; Cc1ccc(-c2nnc(N[C@@H]3CCCN(C)C3)c3cccc(C)c23)c(O)c1 ; Cc1ccc(-c2nnc(N[C@@H]3CCCN(C)C3)c3c(C)cccc23)c(O)c1]

A mixture (1.0 g, 2.87 mmol, 1.0 eq.) of (R)-5-methyl-2-(8-methyl-4-(piperidin-3-ylamino)phthalazin-1-yl)phenol and (R)-5-methyl-2-(5-methyl-4-(piperidin-3-ylamino)phthalazin-1-yl)phenol was dissolved in methanol (10 mL), followed by addition of an aqueous formaldehyde solution (37%) (256 mg, 3.16 mmol, 1.1 eq.). The resulting mixture was stirred at room temperature for 20 min, followed by addition of sodium cyanoborohydride (198 mg, 3.16 mmol, 1.1 eq.). The mixture was reacted at room temperature for 20 min. After the reaction was completed as monitored by LC-MS, the reaction solution was concentrated under reduced pressure, and water (20 mL) was added to the crude product, followed by extraction with DCM (20 mL×3). The organic phase was dried and concentrated, and the crude product was purified by preparative thin-layer chromatography (DCM:MeOH=8:1) to give the product (compound 2) (R)-5-methyl-2-(8-methyl-4-((1-methylpiperidin-3-yl)amino)phthalazin-1-yl)phenol having a smaller Rf value (70 mg, yield: 6.7%).

¹HNMR (400 MHz, DMSO-d6) δ (ppm): 9.31 (s, 1H), 8.31 (s, 1H), 7.74-7.72 (d, 1H), 7.60-7.58 (d, 1H), 7.31 (s, 1H), 7.06-7.03 (m, 1H), 6.73 (s, 2H), 4.68 (s, 1H), 2.57 (s, 4H), 2.50 (s, 3H), 2.31 (s, 3H), 2.06-1.99 (t, 5H), 1.83-1.81 (m, 2H).

Molecular formula: C₂₁H₂₆N₄O molecular weight: 362.48 LC-MS (Pos, m/z)=363.26 [M+H]⁺.

Simultaneously, the product (compound 5) (R)-5-methyl-2-(5-methyl-4-((1-methylpiperidin-3-yl)amino)phthalazin-1-yl)phenol with a larger Rf value (40 mg, yield: 3.8%) was also given.

¹HNMR (400 MHz, DMSO-d6) δ (ppm): 9.51 (s, 1H), 7.63-7.61 (d, 2H), 7.34-7.32 (t, 1H), 7.13-7.11 (d, 1H), 6.81 (s, 1H), 6.77-6.75 (d, 1H), 6.17 (s, 1H), 4.57 (s, 1H), 2.97 (s, 4H), 2.58 (s, 3H), 2.50 (s, 3H), 2.30 (s, 3H), 1.98-1.96 (d, 2H), 1.77 (s, 2H).

Molecular formula: C₂₁H₂₆N₄O molecular weight: 362.48 LC-MS (Pos, m/z)=363.29 [M+H]⁺.

### Example 3: Synthesis of (R)-5-methyl-2-(6-methyl-4-((1-methylpiperidin-3-yl)amino)phthalazin-1-yl)phenol (Compound 4) and (R)-5-methyl-2-(7-methyl-4-((1-methylpiperidin-3-yl)amino)phthalazin-1-yl)phenol (Compound 3)

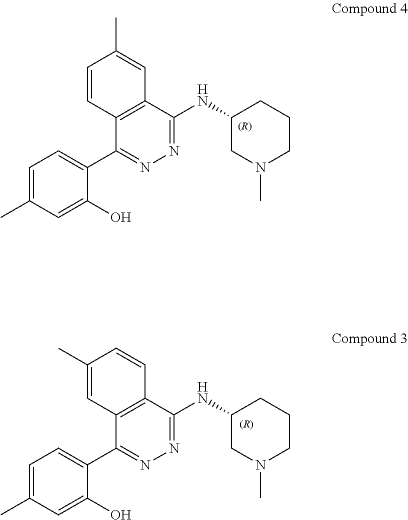

### Step 1: Synthesis of 2-(2-methoxy-4-methylbenzoyl)-5-methylbenzoic acid and 2-(2-methoxy-4-methylbenzoyl)-4-methylbenzoic acid

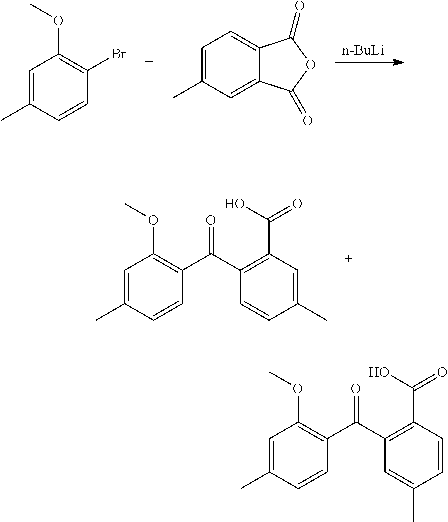

1-Bromo-2-methoxy-4-methylbenzene (13.64 g, 67.84 mmol, 1.1 eq.) was dissolved in THE (50 mL), the mixture was cooled to −50° C., followed by dropwise and slow addition of a solution (2.5 mol/L, 27 mL, 67.84 mmol, 1.1 eq.) of n-butyllithium in n-hexane, and the resulting mixture was stirred at −50° C. for 30 min. A solution of 5-methylisobenzofuran-1,3-dione (10 g, 61.68 mmol, 1.0 eq.) in THE (100 mL) was added dropwise and slowly to the above solution at −60° C., and the reaction solution was reacted at −60° C. for 30 min in nitrogen atmosphere. After the completion of dropwise addition, the reaction solution was naturally warmed to room temperature and reacted for 3 h, and poured into a dilute hydrochloric acid solution (200 mL), followed by addition of EA (100 mL×2) for extraction. The organic phase was washed with 0.2 mol/L NaOH solution (100 mL×2), and hydrochloric acid was added to the aqueous phase to adjust the pH to 4, followed by extraction with EA (100 mL×2). The organic phase was dried and concentrated to give a mixture (17.51 g, yield: 100%) of products.

### Step 2: Synthesis of methyl 2-(2-methoxy-4-methylbenzoyl)-5-methylbenzoate and methyl 2-(2-methoxy-4-methylbenzoyl)-4-methylbenzoate

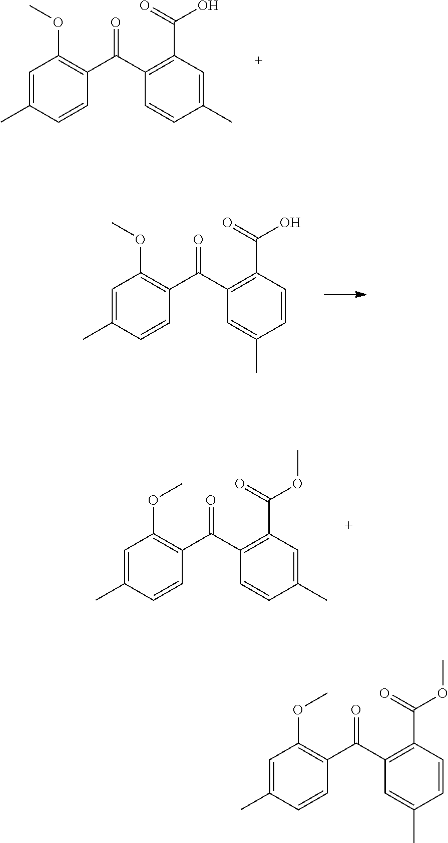

A mixture (17.54 g, 61.68 mmol, 1.0 eq.) of 2-(2-methoxy-4-methylbenzoyl)-5-methylbenzoic acid and 2-(2-methoxy-4-methylbenzoyl)-4-methylbenzoic acid, potassium carbonate (12.83 g, 92.97 mmol, 1.5 eq.) and iodomethane (13.20 g, 92.97 mmol, 1.5 eq.) were dissolved in DMF (100 mL), and the mixture was reacted at room temperature for 30 min. After the reaction was completed as monitored by TLC, the reaction solution was poured into water (200 mL), followed by extraction with methyl tert-butyl ether (150 mL×3). The organic phase was washed with a saturated aqueous NaCl solution (100 mL×2), dried, and concentrated to give a mixture (15.0 g, yield: 82.8%) of methyl 2-(2-methoxy-4-methylbenzoyl)-5-methylbenzoate and methyl 2-(2-methoxy-4-methylbenzoyl)-4-methylbenzoate.

### Step 3: Synthesis of 4-(2-methoxy-4-methylphenyl)-7-methylphthalazin-1(2H)-one and 4-(2-methoxy-4-methylphenyl)-6-methylphthalazin-1(2H)-one

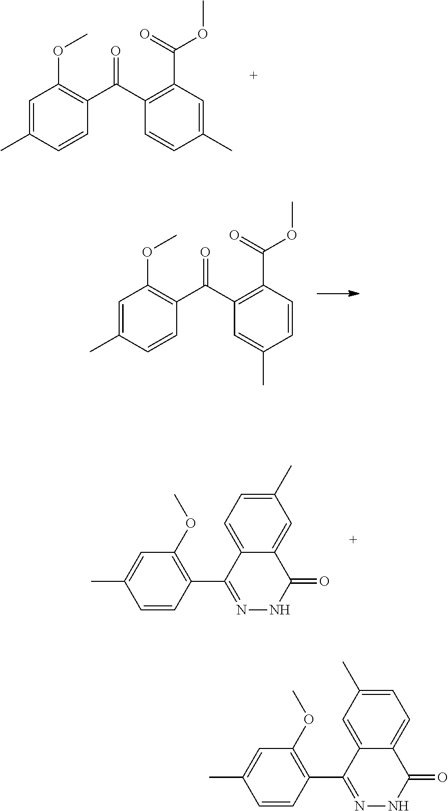

A mixture (15 g, 50.28 mmol, 1.0 eq.) of methyl 2-(2-methoxy-4-methylbenzoyl)-5-methylbenzoate and methyl 2-(2-methoxy-4-methylbenzoyl)-4-methylbenzoate, and 85% hydrazine hydrate (4.44 g, 75.42 mmol, 1.5 eq.) were dissolved in ethanol (150 mL), and the mixture was reacted at 80° C. for 6 h. After the reaction was completed as detected by LC-MS, the reaction solution was cooled to room temperature and filtered, and the filter cake was dried at 50° C. to give a mixture (11.0 g, yield: 78.0%) of 4-(2-methoxy-4-methylphenyl)-7-methylphthalazin-1(2H)-one and 4-(2-methoxy-4-methylphenyl)-6-methylphthalazin-1(2H)-one.

Step 4: Synthesis of 4-chloro-1-(2-methoxy-4-methylphenyl)-6-methylphthalazine and 1-chloro-4-(2-methoxy-4-methylphenyl)-6-methylphthalazine

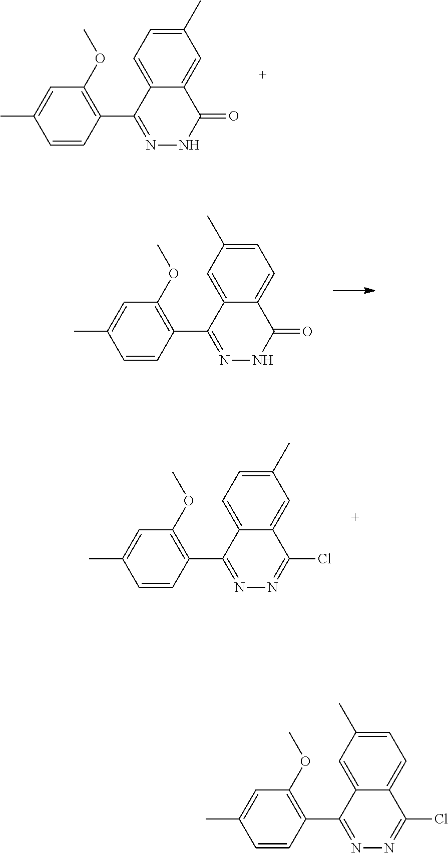 [SMILES: COc1cc(C)ccc1-c1n[nH]c(=O)c2cc(C)ccc12 ; COc1cc(C)ccc1-c1n[nH]c(=O)c2ccc(C)cc12 ; COc1cc(C)ccc1-c1nnc(Cl)c2cc(C)ccc12 ; COc1cc(C)ccc1-c1nnc(Cl)c2ccc(C)cc12]

The mixture (11.0 g, 39.24 mmol, 1.0 eq.) of 4-(2-methoxy-4-methylphenyl)-7-methylphthalazin-1(2H)-one and 4-(2-methoxy-4-methylphenyl)-6-methylphthalazin-1(2H)-one and phosphorus oxychloride (12.0 g, 78.48 mmol, 2.0 eq.) were dissolved in acetonitrile (100 mL), and the resulting mixture was reacted at 90° C. for 2 h. After the reaction was completed as monitored by TLC, the reaction solution was concentrated under reduced pressure, water (50 mL) was added, and EA (100 mL×2) was added for extraction. The organic phase was dried and concentrated to give a mixture (10.0 g, yield: 85.3%) of 4-chloro-1-(2-methoxy-4-methylphenyl)-6-methylphthalazine and 1-chloro-4-(2-methoxy-4-methylphenyl)-6-methylphthalazine.

### Step 5: Synthesis of tert-butyl (R)-3-((4-(2-methoxy-4-methylphenyl)-7-methylphthalazin-1-yl)amino)piperidine-1-carboxylate and tert-butyl (R)-3-((4-(2-methoxy-4-methylphenyl)-6-methylphthalazin-1-yl)amino)piperidine-1-carboxylate

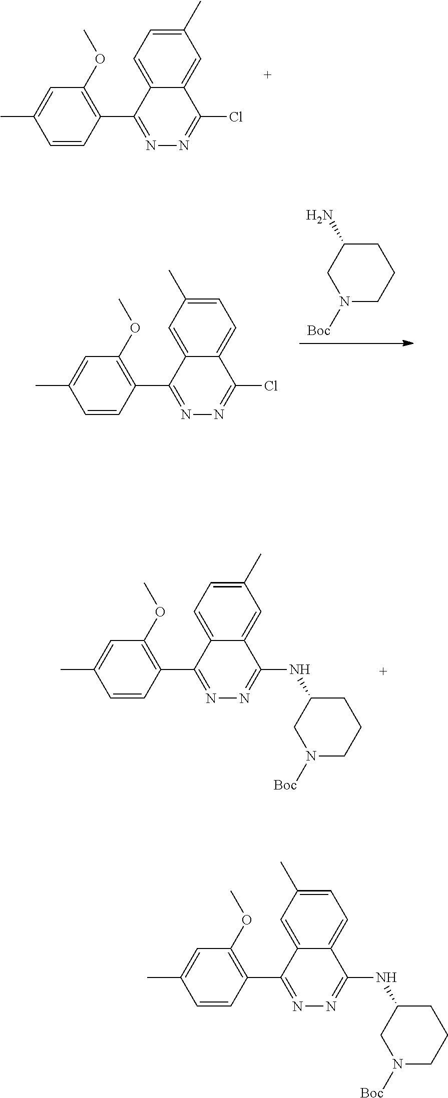 [SMILES: COc1cc(C)ccc1-c1nnc(Cl)c2cc(C)ccc12 ; COc1cc(C)ccc1-c1nnc(Cl)c2ccc(C)cc12 ; CC(C)(C)OC(=O)N1CCC[C@@H](N)C1 ; COc1cc(C)ccc1-c1nnc(N[C@@H]2CCCN(C(=O)OC(C)(C)C)C2)c2cc(C)ccc12 ; COc1cc(C)ccc1-c1nnc(N[C@@H]2CCCN(C(=O)OC(C)(C)C)C2)c2ccc(C)cc12]

The mixture (2.0 g, 6.69 mmol, 1.0 eq.) of 4-chloro-1-(2-methoxy-4-methylphenyl)-6-methylphthalazine and 1-chloro-4-(2-methoxy-4-methylphenyl)-6-methylphthalazine and tert-butyl (R)-3-aminopiperidine-1-carboxylate (2.68 g, 13.38 mmol, 2.0 eq.) were dissolved in DMAC (20 mL), and the resulting mixture was reacted at 120° C. for 19 h. After no materials were left as monitored by TLC, the reaction solution was poured into water (100 mL) and solids precipitated. The mixture was filtered under vacuum, and the filter cake was dried at 60° C. to give a mixture (2.0 g, yield: 64.7%) of tert-butyl (R)-3-((4-(2-methoxy-4-methylphenyl)-7-methylphthalazin-1-yl)amino)piperidine-1-carboxylate and tert-butyl (R)-3-((4-(2-methoxy-4-methylphenyl)-6-methylphthalazin-1-yl)amino)piperidine-1-carboxylate.

### Step 6: Synthesis of (R)-5-methyl-2-(6-methyl-4-(piperidin-3-ylamino)phthalazin-1-yl)phenol and (R)-5-methyl-2-(7-methyl-4-(piperidin-3-ylamino)phthalazin-1-yl)phenol

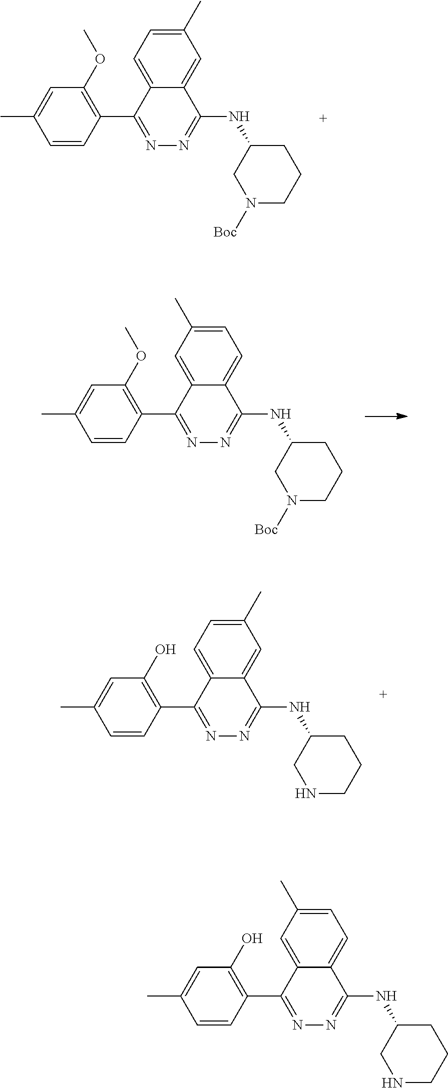 [SMILES: COc1cc(C)ccc1-c1nnc(N[C@@H]2CCCN(C(=O)OC(C)(C)C)C2)c2cc(C)ccc12 ; COc1cc(C)ccc1-c1nnc(N[C@@H]2CCCN(C(=O)OC(C)(C)C)C2)c2ccc(C)cc12 ; Cc1ccc(-c2nnc(N[C@@H]3CCCNC3)c3cc(C)ccc23)c(O)c1 ; Cc1ccc(-c2nnc(N[C@@H]3CCCNC3)c3ccc(C)cc23)c(O)c1]

The mixture (2.0 g, 4.32 mmol, 1.0 eq.) of tert-butyl (R)-3-((4-(2-methoxy-4-methylphenyl)-7-methylphthalazin-1-yl)amino)piperidine-1-carboxylate and tert-butyl (R)-3-((4-(2-methoxy-4-methylphenyl)-6-methylphthalazin-1-yl)amino)piperidine-1-carboxylate was dissolved in DCM (20 mL), and the resulting mixture was cooled to −20° C., followed by dropwise addition of a 1 mol/L solution of boron tribromide in dichloromethane (21.6 mL, 21.60 mmol, 5.0 eq.). After the completion of dropwise addition, the mixture was naturally warmed to room temperature and reacted for 2 h. After the reaction was completed as detected by LC-MS, an appropriate amount of methanol was added into the flask to quench the reaction. The reaction solution was stirred for 10 min and concentrated under reduced pressure. The crude product was purified by silica gel column chromatography (DCM:MeOH=15:1) to give a mixture (1.2 g, yield: 80.0%) of (R)-5-methyl-2-(6-methyl-4-(piperidin-3-ylamino)phthalazin-1-yl)phenol and (R)-5-methyl-2-(7-methyl-4-(piperidin-3-ylamino)phthalazin-1-yl)phenol.

### Step 7: Synthesis of (R)-5-methyl-2-(6-methyl-4-((1-methylpiperidin-3-yl)amino)phthalazin-1-yl)phenol (Compound 4) and (R)-5-methyl-2-(7-methyl-4-((1-methylpiperidin-3-yl)amino)phthalazin-1-yl)phenol (Compound 3)

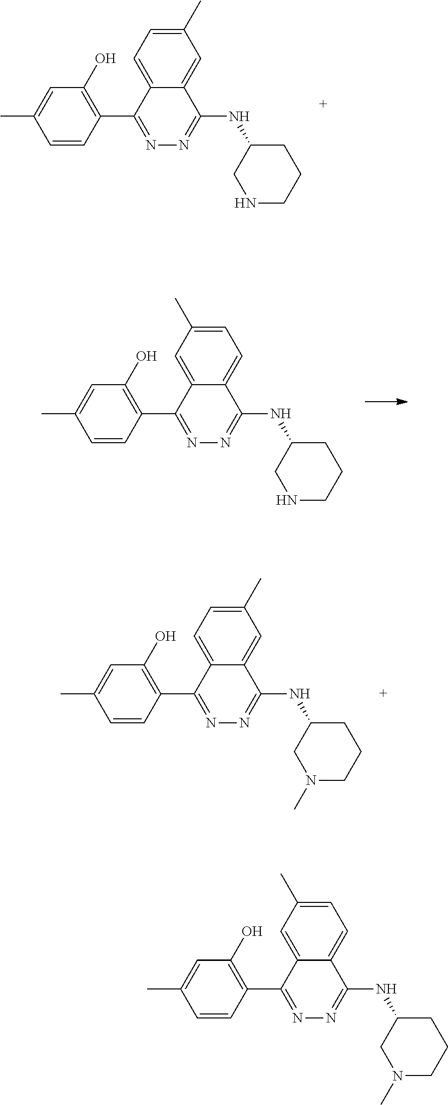 [SMILES: Cc1ccc(-c2nnc(N[C@@H]3CCCNC3)c3cc(C)ccc23)c(O)c1 ; Cc1ccc(-c2nnc(N[C@@H]3CCCNC3)c3ccc(C)cc23)c(O)c1 ; Cc1ccc(-c2nnc(N[C@@H]3CCCN(C)C3)c3cc(C)ccc23)c(O)c1 ; Cc1ccc(-c2nnc(N[C@@H]3CCCN(C)C3)c3ccc(C)cc23)c(O)c1]

The mixture (1.2 g, 3.44 mmol, 1.0 eq.) of (R)-5-methyl-2-(6-methyl-4-(piperidin-3-ylamino)phthalazin-1-yl)phenol and (R)-5-methyl-2-(7-methyl-4-(piperidin-3-ylamino)phthalazin-1-yl)phenol was dissolved in methanol (15 mL), followed by addition of an aqueous formaldehyde solution (37%) (838 mg, 10.32 mmol, 3.0 eq.). The resulting mixture was stirred at room temperature for 30 min, followed by addition of sodium cyanoborohydride (648 mg, 10.32 mmol, 3.0 eq.). The mixture was reacted at room temperature for 30 min. After the reaction was completed as monitored by LC-MS, the reaction solution was concentrated under reduced pressure. Water (20 mL) was added to the crude product, and DCM (20 mL×3) was added for extraction. The organic phase was dried and concentrated to give the crude product (1.0 g). 300 mg of the crude product was purified by preparative thin-layer chromatography (dichloromethane:methanol:ammonia solution=20:1:0.5) to give the product (compound 4) (R)-5-methyl-2-(6-methyl-4-((1-methylpiperidin-3-yl)amino)phthalazin-1-yl)phenol with a larger Rf value (35 mg, yield: 9.4%).

¹HNMR (400 MHz, DMSO-d6) δ (ppm): 9.68 (s, 1H), 8.18 (s, 1H), 7.61-7.58 (d, 1H), 7.46-7.43 (d, 1H), 7.17-7.15 (d, 1H), 6.91-6.89 (d, 1H), 6.79-6.75 (t, 2H), 4.40-4.38 (t, 1H), 3.08-3.06 (d, 1H), 2.72-2.69 (d, 1H), 2.52 (s, 3H), 2.32 (s, 3H), 2.21 (s, 3H), 1.99-1.92 (m, 3H), 1.75-1.74 (d, 1H), 1.65-1.56 (m, 1H), 1.49-1.43 (m, 1H).

Molecular formula: C₂₁H₂₆N₄O Molecular weight: 362.48 LC-MS (Pos, m/z)=363.32[M+H]⁺.

Simultaneously, the product (compound 3) (R)-5-methyl-2-(7-methyl-4-((1-methylpiperidin-3-yl)amino)phthalazin-1-yl)phenol with a smaller Rf value (100 mg, yield: 26.8%) was also given.

¹HNMR (400 MHz, DMSO-d6) δ (ppm): 9.62 (s, 1H), 8.29-8.27 (d, 1H), 7.68-7.66 (d, 1H), 7.30 (s, 1H), 7.16-7.14 (d, 1H), 7.00-6.99 (d, 1H), 6.80 (s, 1H), 6.77-6.75 (d, 1H), 4.43-4.42 (d, 1H), 3.13-3.15 (d, 1H), 2.79-2.76 (d, 1H), 2.42 (s, 3H), 2.33 (s, 3H), 2.28 (s, 3H), 2.03-1.97 (m, 3H), 1.80-1.76 (t, 1H), 1.64-1.61 (d, 1H), 1.50-1.47 (d, 1H).

Molecular formula: C₂₁H₂₆N₄O Molecular weight: 362.48 LC-MS (Pos, m/z)=363.32[M+H]⁺.

### Example 4: Synthesis of (R)-2-(5-bromo-4-((1-methylpiperidin-3-yl)amino)phthalazin-1-yl)-5-methylphenol (Compound 34)

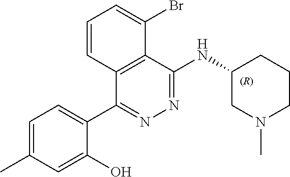

### Step 1: Synthesis of 2-bromo-6-(2-methoxy-4-methylbenzoyl)benzoic acid

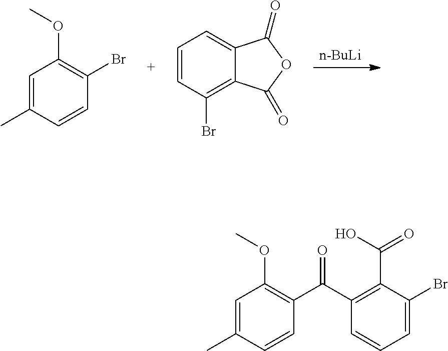

1-Bromo-2-methoxy-4-methylbenzene (4.87 g, 24.22 mmol, 1.1 eq.) was dissolved in THE (50 mL), and the mixture was cooled to −60° C., followed by dropwise and slow addition of a solution of n-butyllithium in n-hexane (2.5 mol/L, 9.7 mL, 24.22 mmol, 1.1 eq.). The reaction solution was stirred at −60° C. for 30 min and then added dropwise to a solution of 4-bromoisobenzofuran-1,3-dione (5 g, 22.02 mmol, 1.0 eq.) in THE (50 mL) at −60° C. in nitrogen atmosphere. After the completion of dropwise addition, the resulting mixture was naturally warmed to room temperature and reacted for 4 h. After the reaction was completed as detected by LC-MS, the reaction solution was added into a dilute hydrochloric acid solution (100 mL), and EA (50 mL×2) was added for extraction. The organic phase was washed with a 0.2 mol/L NaOH solution (50 mL×2), and the pH of the aqueous phase was adjusted to 4 with hydrochloric acid, and EA (100 mL×2) was added for extraction. The organic phase was dried and concentrated to give the product (5.0 g, yield: 65.1%).

### Step 2: Synthesis of methyl 2-bromo-6-(2-methoxy-4-methylbenzoyl)benzoate

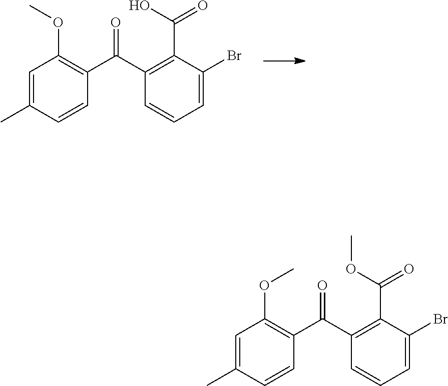 [SMILES: COc1cc(C)ccc1C(=O)c1cccc(Br)c1C(=O)O ; COC(=O)c1c(Br)cccc1C(=O)c1ccc(C)cc1OC]

2-Bromo-6-(2-methoxy-4-methylbenzoyl)benzoic acid (5.0 g, 14.32 mmol, 1.0 eq.), potassium carbonate (2.96 g, 21.48 mmol, 1.5 eq.) and iodomethane (3.05 g, 21.48 mmol, 1.5 eq.) were dissolved in DMF (50 mL), and the mixture was reacted at room temperature for 0.5 h. After the reaction was completed as monitored by LC-MS, the reaction solution was poured into water (100 mL), and methyl tert-butyl ether (100 mL×2) was added for extraction. The organic phase was washed with water (50 mL×2), dried and concentrated to give the product (4.0 g, yield: 76.9%).

### Step 3: Synthesis of 8-bromo-4-(2-methoxy-4-methylphenyl)phthalazin-1(2H)-one

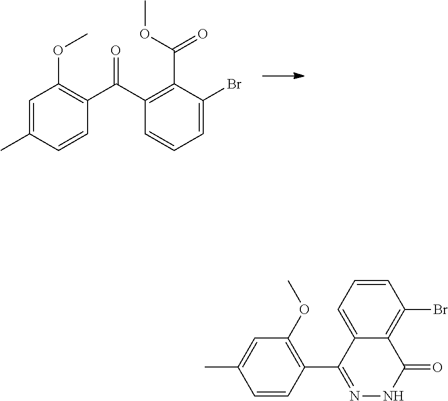 [SMILES: COC(=O)c1c(Br)cccc1C(=O)c1ccc(C)cc1OC ; COc1cc(C)ccc1-c1n[nH]c(=O)c2c(Br)cccc12]

Methyl 2-bromo-6-(2-methoxy-4-methylbenzoyl)benzoate (4.0 g, 11.01 mmol, 1.0 eq.) and 85% hydrazine hydrate (973 mg, 16.52 mmol, 1.5 eq.) were dissolved in ethanol (40 mL), and the mixture was reacted at 80° C. for 15 h. A large number of solids precipitated, and the mixture was filtered. The filter cake was dried to give the product (1.4 g, yield: 36.8%).

### Step 4: Synthesis of 5-bromo-4-chloro-1-(2-methoxy-4-methylphenyl)phthalazine

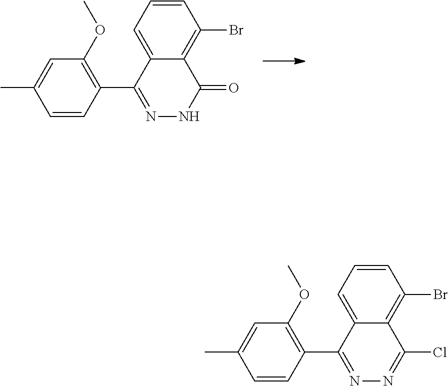 [SMILES: COc1cc(C)ccc1-c1n[nH]c(=O)c2c(Br)cccc12 ; COc1cc(C)ccc1-c1nnc(Cl)c2c(Br)cccc12]

Methyl 2-bromo-6-(2-methoxy-4-methylbenzoyl)benzoate (800 mg, 2.32 mmol, 1.0 eq.) and phosphorus oxychloride (711 mg, 28.52 mmol, 2.0 eq.) were dissolved in acetonitrile (10 mL), and the mixture was reacted at 90° C. for 1 h. After the reaction was completed as monitored by TLC, the reaction solution was concentrated under reduced pressure, the crude product was dissolved in EA (20 mL), and water (10 mL) was added. The mixture was stirred for 5 min, followed by liquid separation. The aqueous phase was extracted with EA (20 mL), and the organic phases were combined, dried and concentrated under reduced pressure to give the product (600 mg, yield: 71.2%).

### Step 5: Synthesis of tert-butyl (R)-3-((8-bromo-4-(2-methoxy-4-methylphenyl)phthalazin-1-yl)amino)piperidine-1-carboxylate

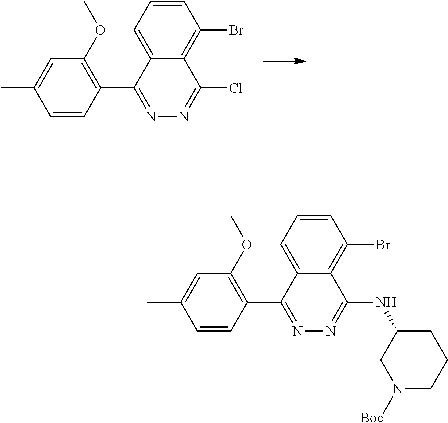 [SMILES: COc1cc(C)ccc1-c1nnc(Cl)c2c(Br)cccc12 ; COc1cc(C)ccc1-c1nnc(N[C@@H]2CCCN(C(=O)OC(C)(C)C)C2)c2c(Br)cccc12]

5-bromo-4-chloro-1-(2-methoxy-4-methylphenyl)phthalazine (600 mg, 1.65 mmol, 1.0 eq.) and tert-butyl (R)-3-aminopiperidine-1-carboxylate (660 mg, 3.30 mmol, 2.0 eq.) were dissolved in DMAC (5 mL), and the mixture was reacted at 120° C. for 20 h. After the reaction was completed as monitored by TLC, the reaction solution was cooled to room temperature and poured into water (30 mL). Solids precipitated, and the mixture was filtered under vacuum. The filter cake was dried to give the product (700 mg, yield: 80.4%).

### Step 6: Synthesis of (R)-2-(5-bromo-4-(piperidin-3-ylamino)phthalazin-1-yl)-5-methylphenol

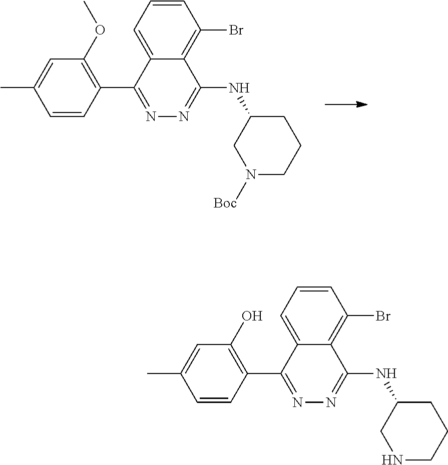 [SMILES: COc1cc(C)ccc1-c1nnc(N[C@@H]2CCCN(C(=O)OC(C)(C)C)C2)c2c(Br)cccc12 ; Cc1ccc(-c2nnc(N[C@@H]3CCCNC3)c3c(Br)cccc23)c(O)c1]

Tert-butyl (R)-3-((8-bromo-4-(2-methoxy-4-methylphenyl)phthalazin-1-yl)amino)piperidine-1-carboxylate (700 mg, 1.32 mmol, 1.0 eq.) was dissolved in DCM (10 mL), and the mixture was cooled to −20° C., followed by dropwise addition of a 1 mol/L solution of boron tribromide in dichloromethane (6.6 mL, 6.60 mmol, 5.0 eq.). After the completion of dropwise addition, the mixture was naturally warmed to room temperature and reacted for 3 h. After the reaction was completed as detected by LC-MS, an appropriate amount of methanol was added to quench the reaction. The reaction solution was concentrated under reduced pressure, and the crude product was dissolved in DCM (10 mL), followed by addition of water (10 mL) and liquid separation. The aqueous phase was extracted with DCM (10 mL×2), and the organic phases were combined, dried and concentrated to give the product (280 mg, yield: 51.4%).

### Step 7: Synthesis of (R)-2-(5-bromo-4-((1-methylpiperidin-3-yl)amino)phthalazin-1-yl)-5-methylphenol

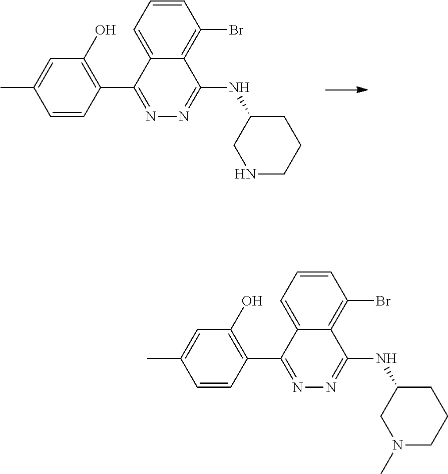 [SMILES: Cc1ccc(-c2nnc(N[C@@H]3CCCNC3)c3c(Br)cccc23)c(O)c1 ; Cc1ccc(-c2nnc(N[C@@H]3CCCN(C)C3)c3c(Br)cccc23)c(O)c1]

(R)-2-(5-bromo-4-(piperidin-3-ylamino)phthalazin-1-yl)-5-methylphenol (250 mg, 0.60 mmol, 1.0 eq.) was dissolved in methanol (5 mL), followed by addition of an aqueous formaldehyde solution (37%) (146 mg, 1.80 mmol, 3.0 eq.). The mixture was stirred at room temperature for 30 min, followed by addition of sodium cyanoborohydride (113 mg, 1.80 mmol, 3.0 eq.). The mixture was reacted at room temperature for 30 min. After no materials were left as monitored by LC-MS, the reaction solution was concentrated. The crude product was slurried with water (10 mL) and solids precipitated. The mixture was filtered, and the filter cake was dissolved in EA (10 mL), dried and filtered. The filtrate was concentrated under reduced pressure, and the crude product was purified by preparative thin-layer chromatography (DCM:MeOH=10:1) to give the product (120 mg, yield: 46.8%).

¹HNMR (300 MHz, DMSO-d6) δ (ppm): 9.50 (s, 1H), 8.15-8.13 (m, 1H), 7.66-7.61 (t, 1H), 7.50-7.47 (m, 2H), 7.16-7.13 (d, 1H), 6.80-6.76 (t, 2H), 4.53 (s, 1H), 2.80-2.75 (t, 1H), 2.58 (d, 2H), 2.33 (s, 7H), 1.79 (s, 3H), 1.65 (s, 1H).

Molecular formula: C₂₁H₂₃Br N₄O Molecular weight: 427.35 LC-MS (Pos, m/z)=427.25/429.25[M+H]⁺.

### Example 5: Synthesis of (R)-4,5-dimethyl-2-(4-((1-methylpiperidin-3-yl)amino)phthalazin-1-yl)phenol (Compound 35)

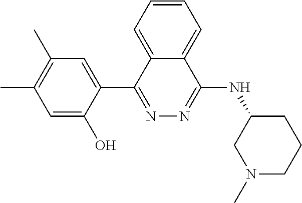 [SMILES: Cc1cc(O)c(-c2nnc(N[C@@H]3CCCN(C)C3)c3ccccc23)cc1C]

### Step 1: Synthesis of 2-bromo-4,5-dimethylphenol

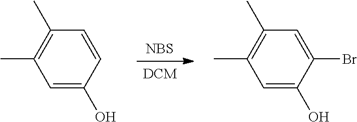

3,4-Dimethylphenol (5.0 g, 40.9 mmol, 1.0 eq.) was dissolved in DCM (50 mL), and NBS (7.28 g, 40.9 mmol, 1.0 eq.) was added portionwise under an ice-water bath. The mixture was reacted for 2 h. The reaction solution was washed with water, and the organic phase was dried over anhydrous sodium sulfate and filtered. The filtrate was concentrated under reduced pressure, and the crude product was purified by silica gel column chromatography (PE:EA=50:1 to 20:1) to give the product (7.20 g, yield: 87.5%).

### Step 2: Synthesis of 1-bromo-2-methoxy-4,5-dimethylbenzene

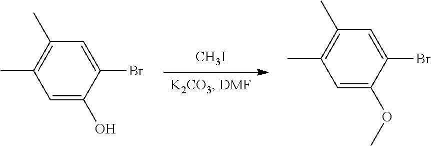

2-Bromo-4,5-dimethylphenol (7.20 g, 35.8 mmol, 1.0 eq.) was dissolved in DMF (80 mL), and then K₂CO₃ (9.90 g, 71.6 mmol, 2.0 eq.) and CH₃I (7.62 g, 53.7 mmol, 1.5 eq.) were added. The mixture was stirred at room temperature for 3 h. Water was added to quench the reaction, and EA (50 mL) was added for extraction. The organic phase was dried over anhydrous sodium sulfate and filtered, and the filtrate was concentrated under reduced pressure. The crude product was purified by silica gel column chromatography (PE:EA=50:1 to 20:1) to give the product (7.10 g, yield: 92.2%).

### Step 3: Synthesis of (2-methoxy-4,5-dimethylphenyl)boronic acid

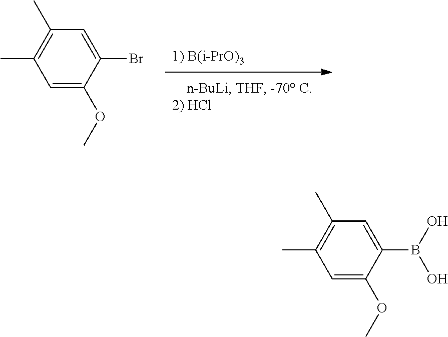

1-Bromo-2-methoxy-4,5-dimethylbenzene (5.00 g, 23.2 mmol, 1.0 eq.) and triisopropyl borate (8.73 g, 46.4 mmol, 2.0 eq.) were dissolved in THF (50 mL), followed by purge with nitrogen for protection. The mixture was cooled to −70° C., followed by dropwise addition of n-butyllithium (18.6 mL, 46.4 mmol, 2.0 eq.). The mixture was reacted for 3 h. Dilute hydrochloric acid was added to quench the reaction, the pH was adjusted to 2, and EA (50 mL) was added for extraction. The organic phase was dried over anhydrous sodium sulfate and filtered, and the filtrate was concentrated under reduced pressure and slurried with petroleum ether to give the product (2.60 g, yield: 62.1%).

### Step 4: Synthesis of (R)-4-(2-methoxy-4,5-dimethylphenyl)-N-(1-methylpiperidin-3-yl)phthalazin-1-amine

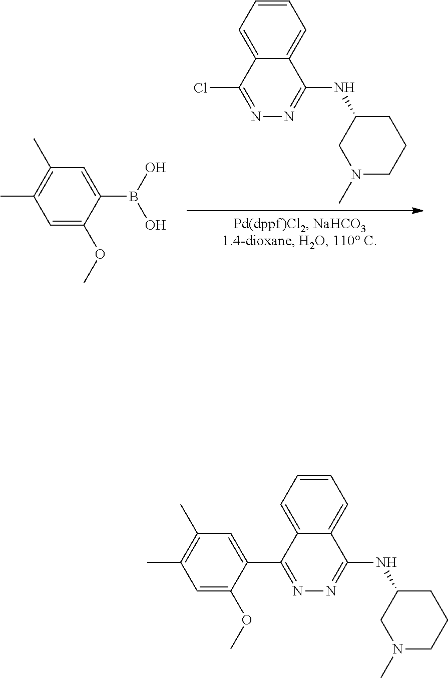 [SMILES: COc1cc(C)c(C)cc1B(O)O ; CN1CCC[C@@H](Nc2nnc(Cl)c3ccccc23)C1 ; "Pd(dppf)Cl2, NaHCO31.4-dioxane, H2O, 110° C." ; COc1cc(C)c(C)cc1-c1nnc(N[C@@H]2CCCN(C)C2)c2ccccc12]

(2-Methoxy-4,5-dimethylphenyl)boronic acid (194 mg, 1.08 mmol, 1.5 eq.), (R)-4-chloro-N-(1-methylpiperidin-3-yl)phthalazin-1-amine (200 mg, 0.723 mmol, 1.0 eq.), Pd(dppf)Cl₂ (26.5 mg, 0.0362 mmol, 0.05 eq.) and NaHCO₃ (122 mg, 1.45 mmol, 2.0 eq.) were added successively to 1,4-dioxane (4 mL), and H₂O (2 mL) was added, followed by purge with nitrogen for protection. The oil bath was heated to 110° C. and the mixture was reacted for 1 h. The reaction solution was cooled down, water was added to quench the reaction, and EA (20 mL) was added for extraction. The organic phase was dried over anhydrous sodium sulfate and filtered, and the filtrate was concentrated under reduced pressure. The crude product was purified by silica gel column chromatography (DCM:MeOH=50:1 to 20:1) to give the product (200 mg, yield: 73.5%).

### Step 5: Synthesis of (R)-4,5-dimethyl-2-(4-((1-methylpiperidin-3-yl)amino)phthalazin-1-yl)phenol

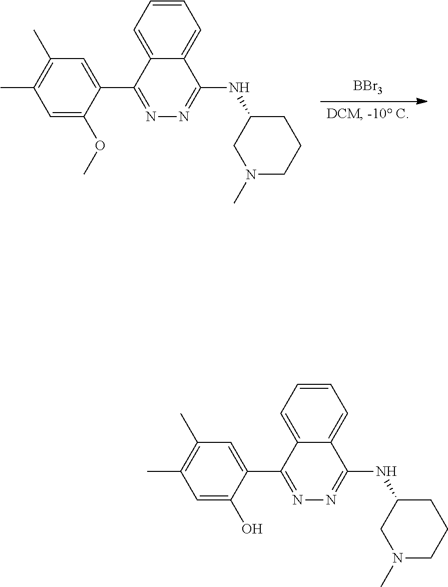 [SMILES: COc1cc(C)c(C)cc1-c1nnc(N[C@@H]2CCCN(C)C2)c2ccccc12 ; "BBr3" ; "DCM, -10° C." ; Cc1cc(O)c(-c2nnc(N[C@@H]3CCCN(C)C3)c3ccccc23)cc1C]

(R)-4-(2-methoxy-4,5-dimethylphenyl)-N-(1-methylpiperidin-3-yl)phthalazin-1-amine (200 mg, 0.531 mmol, 1.0 eq.) was dissolved in DCM (4 mL), and the mixture was cooled to −10° C., followed by dropwise addition of BBr₃ (266 mg, 1.06 mmol, 2.0 eq.). The mixture was reacted for 2 h. MeOH was added to quench the reaction, a saturated aqueous NaHCO₃ solution was added to adjust the pH to 8, and DCM (20 mL) was added for extraction. The organic phase was dried over anhydrous sodium sulfate and filtered, and the filtrate was concentrated under reduced pressure. The crude product was purified by preparative thin-layer chromatography (DCM:MeOH=10:1) to give the product (120 mg, yield: 62.3%).

¹HNMR (300 MHz, DMSO-d6) δ (ppm): 9.37 (s, 1H), 8.41 (d, J=8.1 Hz, 1H), 7.86-7.74 (m, 2H), 7.54 (d, J=7.8 Hz, 1H), 7.14 (s, 1H), 7.04 (s, 1H), 6.80 (s, 1H), 4.49 (s, 1H), 3.21 (d, J=8.4 Hz, 1H), 2.84 (d, J=11.1 Hz, 1H), 2.35 (s, 3H), 2.23-2.18 (m, 8H), 2.02-1.97 (m, 1H), 7.84-1.80 (m, 1H), 1.72-1.49 (m, 2H).

Molecular formula: C₂₂H₂₆N₄O Molecular weight: 362.48 LC-MS (Pos, m/z)=363.34[M+H]⁺.

### Example 6: Synthesis of (R)-5-methyl-2-(4-((1-methylpiperidin-3-yl)amino)thieno[3,4-d]pyridazin-1-yl)phenol (Compound 36)

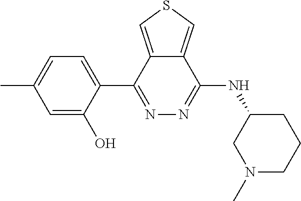 [SMILES: Cc1ccc(-c2nnc(N[C@@H]3CCCN(C)C3)c3cscc23)c(O)c1]

### Step 1: Synthesis of diethyl thiophene-3,4-dicarboxylate

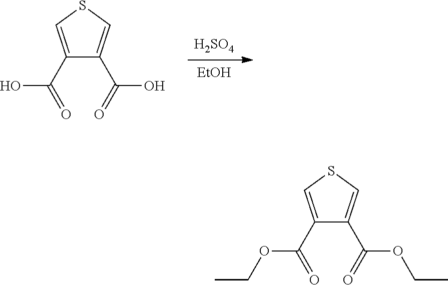

Thiophene-3,4-dicarboxylic acid (5.0 g, 29.0 mmol, 1.0 eq.) was dissolved in EtOH (50 mL), followed by addition of H₂SO₄ (5.69 g, 58.0 mmol, 2.0 eq.). The oil bath was heated to 80° C. and the mixture was reacted for 20 h. The reaction solution was concentrated to remove EtOH, water was added for dissolution, and EA (50 mL) was added for extraction. The organic phase was washed with a saturated aqueous NaHCO₃ solution (40 mL), dried over anhydrous sodium sulfate and filtered, and the filtrate was concentrated under reduced pressure to give the product (6.20 g, yield: 93.5%).

### Step 2: Synthesis of 2,3-dihydrothieno[3,4-d]pyridazine-1,4-dione

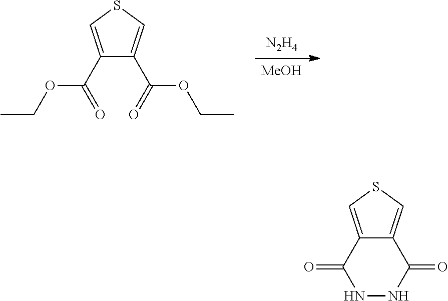

Diethyl thiophene-3,4-dicarboxylate (6.20 g, 27.2 mmol, 1.0 eq.) was dissolved in MeOH (30 mL), followed by addition of hydrazine hydrate (85%, 8.01 g, 136 mmol, 5.0 eq.). The oil bath was heated to 65° C. and the mixture was reacted for 4 h. The reaction solution was cooled to room temperature, filtered under vacuum and dried to give the product (3.48 g, yield: 76.2%).

### Step 3: Synthesis of 1,4-dichlorothieno[3,4-d]pyridazine

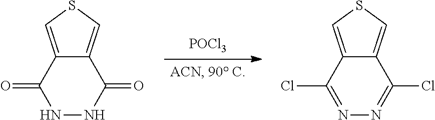 [SMILES: O=c1[nH][nH]c(=O)c2cscc12 ; "POCl3" ; "ACN, 90° C." ; Clc1nnc(Cl)c2cscc12]

2,3-Dihydrothieno[3,4-d]pyridazine-1,4-dione (1.00 g, 5.95 mmol, 1.0 eq.) was added to ACN (10 mL), followed by addition of POCl₃ (2.00 g, 13.1 mmol, 2.2 eq.). The oil bath was heated to 90° C. and the mixture was reacted for 1 h. The reaction solution was cooled to room temperature, water (20 mL) was added to quench the reaction, and EA (20 mL) was added for extraction. The organic phase was dried over anhydrous sodium sulfate and filtered, and the filtrate was concentrated under reduced pressure to give 1,4-dichlorothieno[3,4-d]pyridazine (780 mg, yield: 64.0%).

### Step 4: Synthesis of 4-(2-methoxy-4-methylphenyl)thieno[3,4-d]pyridazin-1-ol

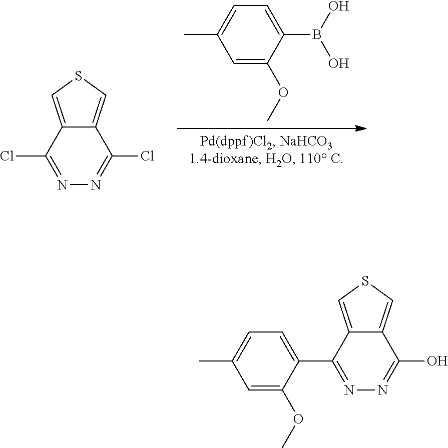

(2-Methoxy-4-methylphenyl)boronic acid (631 mg, 3.8 mmol, 1.5 eq.), 1,4-dichlorothieno[3,4-d]pyridazine (780 mg, 3.80 mmol, 1.0 eq.), Pd(dppf)Cl₂ (139 mg, 0.190 mmol, 0.05 eq.) and NaHCO₃ (1.28 g, 7.60 mmol, 2.0 eq.) were added successively to 1,4-dioxane (10 mL), and H₂O (5 mL) was added, followed by purge with nitrogen for protection. The oil bath was heated to 110° C. and the mixture was reacted for 1 h. Water was added to quench the reaction, and EA (30 mL) was added for extraction. The organic phase was dried over anhydrous sodium sulfate and filtered, and the filtrate was concentrated under reduced pressure. The crude product was separated by silica gel column chromatography (DCM:MeOH=100:1 to 20:1) to give the product (820 mg, yield: 79.2%).

### Step 5: Synthesis of 1-chloro-4-(2-methoxy-4-methylphenyl)thieno[3,4-d]pyridazine

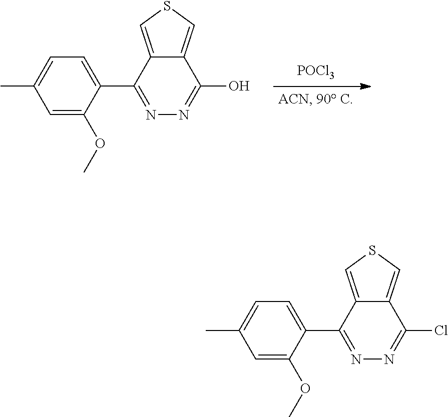 [SMILES: COc1cc(C)ccc1-c1nnc(O)c2cscc12 ; "POCl3" ; "ACN, 90° C." ; COc1cc(C)ccc1-c1nnc(Cl)c2cscc12]

4-(2-Methoxy-4-methylphenyl)thieno[3,4-d]pyridazin-1-ol (820 mg, 3.01 mmol, 1.0 eq.) was added to ACN (10 mL), followed by addition of POCl₃ (693 mg, 4.52 mmol, 1.5 eq.). The oil bath was heated to 90° C. and the mixture was reacted for 1 h. The reaction solution was cooled to room temperature, water was added to quench the reaction, and EA (20 mL) was added for extraction. The organic phase was dried over anhydrous sodium sulfate and filtered, and the filtrate was concentrated under reduced pressure to give the product (680 mg, yield: 77.7%).

### Step 6: Synthesis of tert-butyl (R)-3-((4-(2-methoxy-4-methylphenyl)thieno [3,4-d]pyridazin-1-yl)amino)piperidine-1-carboxylate

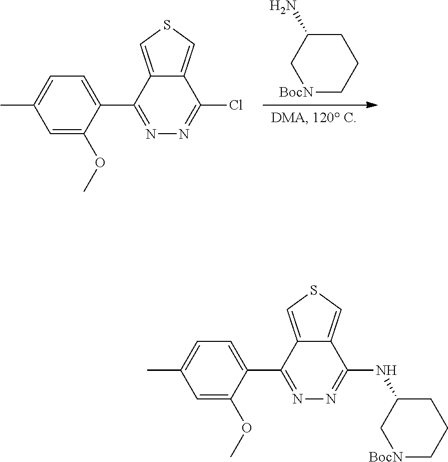

1-Chloro-4-(2-methoxy-4-methylphenyl)thieno[3,4-d]pyridazine (600 mg, 2.06 mmol, 1.0 eq.) and tert-butyl (R)-3-aminopiperidine-1-carboxylate (619 mg, 3.09 mmol, 1.5 eq.) were dissolved in DMA (10 mL), followed by purge with nitrogen for protection. The oil bath was heated to 120° C. and the mixture was reacted for 20 h. Water was added to quench the reaction, and EA (30 mL) was added for extraction. The organic phase was washed three times with water (30 mL×3), dried over anhydrous sodium sulfate and filtered, and the filtrate was concentrated under reduced pressure to give tert-butyl (R)-3-((4-(2-methoxy-4-methylphenyl)thieno[3,4-d]pyridazin-1-yl)amino)piperidine-1-carboxylate (420 mg, yield: 44.8%).

### Step 7: Synthesis of (R)-5-methyl-2-(4-(piperidin-3-ylamino)thieno[3,4-d]pyridazin-1-yl)phenol

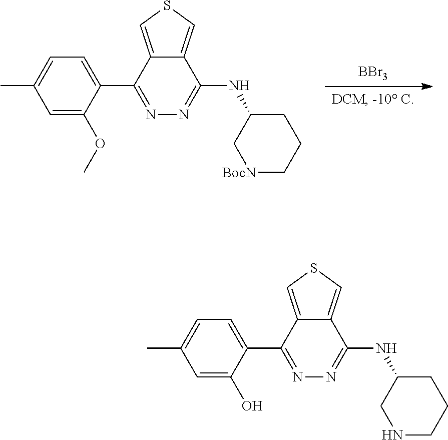 [SMILES: COc1cc(C)ccc1-c1nnc(N[C@@H]2CCCN(C(=O)OC(C)(C)C)C2)c2cscc12 ; "BBr3" ; "DCM, -10° C." ; Cc1ccc(-c2nnc(N[C@@H]3CCCNC3)c3cscc23)c(O)c1]

Tert-butyl (R)-3-((4-(2-methoxy-4-methylphenyl)thieno[3,4-d]pyridazin-1-yl)amino)piperidine-1-carboxylate (420 mg, 0.924 mmol, 1.0 eq.) was dissolved in DCM (8 mL), and the mixture was cooled to −10° C., followed by dropwise addition of BBr₃ (1.16 mg, 4.62 mmol, 5.0 eq.). The mixture was reacted for 2 h. MeOH was added to quench the reaction, and the reaction solution was concentrated. The crude product was dissolved in H₂O and extracted with EA. A saturated aqueous NaHCO₃ solution was added to the aqueous phase to adjust the pH to 8, and DCM (20 mL×3) was added for extraction. The organic phase was dried over anhydrous sodium sulfate and filtered, and the filtrate was concentrated under reduced pressure to give the product (182 mg, yield: 57.9%).

### Step 8: Synthesis of (R)-5-methyl-2-(4-((1-methylpiperidin-3-yl)amino)thieno[3,4-d]pyridazin-1-yl)phenol

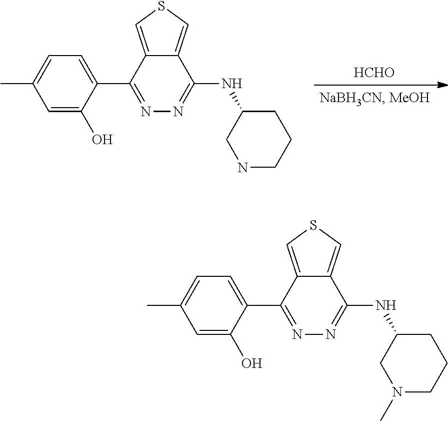

(R)-5-methyl-2-(4-(piperidin-3-ylamino)thieno[3,4-d]pyridazin-1-yl)phenol (180 mg, 0.529 mmol, 1.0 eq.) was dissolved in MeOH (4 mL), followed by addition of an aqueous formaldehyde solution (129 mg, 1.59 mmol, 3.0 eq.). The mixture was stirred at room temperature for 30 min. Sodium cyanoborohydride (100 mg, 1.59 mmol, 3.0 eq.) was then added and the mixture was reacted for 1 h. A saturated aqueous NaHCO₃ solution was added to quench the reaction, and EA (20 mL) was added for extraction. The organic phase was dried over anhydrous sodium sulfate and filtered, and the filtrate was concentrated under reduced pressure. The crude product was separated by preparative thin-layer chromatography to give the product (52.0 mg, yield: 27.7%).

¹HNMR (300 MHz, DMSO-d6) δ (ppm): 13.28 (s, 1H), 9.01 (s, 1H), 8.66 (d, J=2.7 Hz, 1H), 7.87 (d, J=7.8 Hz, 2H), 8.49 (d, J=8.4 Hz, 2H), 4.59 (s, 1H), 3.11 (s, 2H), 2.74 (s, 1H), 2.64 (s, 3H), 2.32 (s, 3H), 1.99-1.74 (m, 5H).

Molecular formula: C₁₉H₂₂N₄OS Molecular weight: 354.47 LC-MS (Pos, m/z)=355.19[M+H]⁺.

### Example 7: Synthesis of (R)-5-methyl-2-(5-((1-methylpiperidin-3-yl)amino)pyrido[2,3-d]pyridazin-8-yl)phenol (Compound 37)

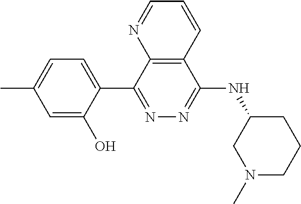 [SMILES: Cc1ccc(-c2nnc(N[C@@H]3CCCN(C)C3)c3cccnc23)c(O)c1]

### Step 1: Synthesis of Intermediate 2-(2-methoxy-4-methylbenzoyl)nicotinic acid

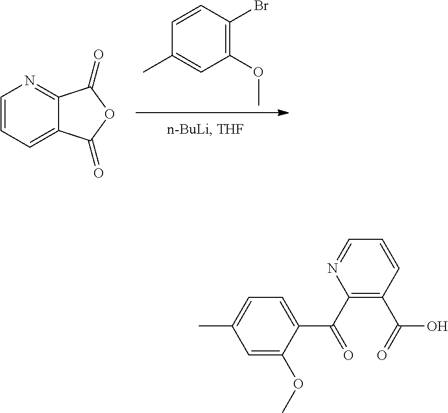

1-Bromo-2-methoxy-4-methylbenzene (14.15 g, 70.38 mmol, 1.05 eq.) was dissolved in anhydrous THE (100 mL), followed by dropwise addition of n-BuLi (33.8 mL, 80.48 mmol, 1.2 eq.) at −78° C. After the completion of dropwise addition, the mixture was reacted for 30 min. At −78° C., the reaction solution was added dropwise to a solution of 2,3-pyridine dicarboxylic anhydride (10 g, 67.07 mmol, 1.0 eq.) in THE (100 mL). After the completion of dropwise addition, the mixture was reacted at room temperature for 16 h. After the reaction was completed as detected by LC-MS, the reaction solution was poured into a mixed solution of ice water (50 mL) and 2 N HCl (50 mL), and ethyl acetate (200 mL) was added for extraction. The aqueous phase was extracted with dichloromethane (200 mL), and the organic phases were combined, dried and concentrated to give the crude product (26 g). The crude product was subjected to silica gel column chromatography (petroleum ether:ethyl acetate=2:1) to give the product (1.9 g, yield: 10.4%).

### Step 2: Synthesis of Intermediate methyl 2-(2-methoxy-4-methylbenzoyl) nicotinate

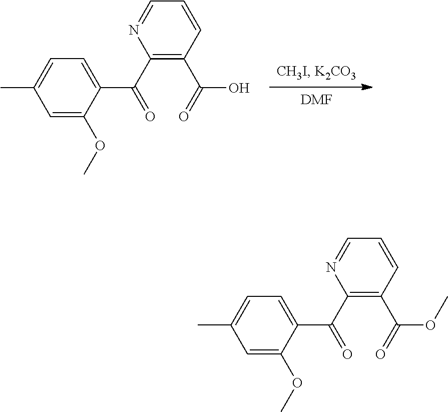

The 2-(2-methoxy-4-methylbenzoyl)nicotinic acid (1.9 g, 7 mmol, 1.0 eq.) obtained in the previous step was dissolved in DMF (15 mL), followed by addition of potassium carbonate (1.45 g, 10.5 mmol, 1.5 eq.) and iodomethane (1.19 g, 8.4 mmol, 1.2 eq.). The mixture was reacted at room temperature for 16 h. After the reaction was completed as detected by TLC, the reaction solution was poured into water (20 mL), and EA (20 mL) was added for extraction. The organic phase was washed with water (20 mL) and saturated brine (20 mL), dried over anhydrous sodium sulfate and concentrated to give the crude product (1.4 g). The crude product was purified by silica gel column chromatography (petroleum ether:ethyl acetate=5:1 to 3:1) to give the product (1.23 g, yield: 61.5%).

### Step 3: Synthesis of Intermediate 8-(2-methoxy-4-methylphenyl)pyrido[2,3-d]pyridazin-5-ol

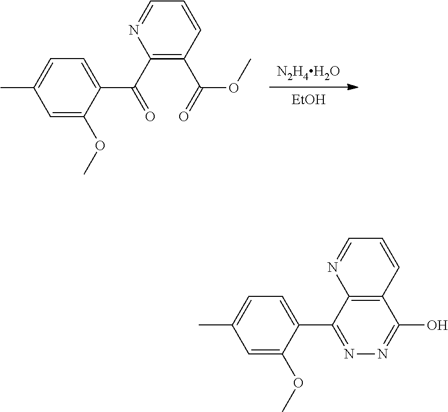

Methyl 2-(2-methoxy-4-methylbenzoyl)nicotinate (1.23 g, 4.31 mmol, 1.0 eq.) obtained in the previous step was dissolved in ethanol (10 mL), followed by addition of 85% hydrazine hydrate (324 mg, 4.47 mmol, 1.5 eq.). The mixture was heated to 85° C. and reacted for 2 h. After the reaction was completed as detected by TLC, the reaction solution was cooled to room temperature, and the product precipitated. The mixture was filtered to give the product (1.02 g, yield: 88.6%).

### Step 4: Synthesis of Intermediate 5-chloro-8-(2-methoxy-4-methylphenyl) pyrido[2,3-d]pyridazine

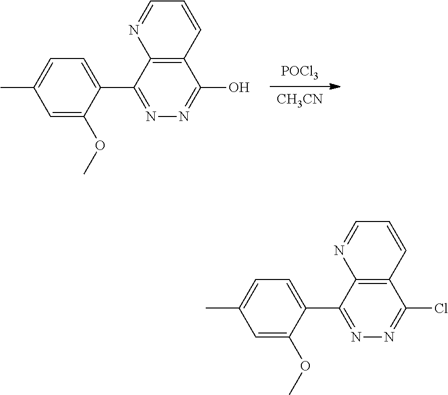

Acetonitrile (10 mL) was added to 8-(2-methoxy-4-methylphenyl)pyrido[2,3-d]pyridazin-5-ol (1.02 g, 3.82 mmol, 1.0 eq.), followed by addition of phosphorus oxychloride (1.17 g, 7.64 mmol, 2.0 eq.). The mixture was stirred at 90° C. for 6 h. After the reaction was completed as detected by TLC, the reaction solution was concentrated. The concentrated solution was poured into ice water (10 mL), and ethyl acetate (10 mL) was added for extraction. The organic phase was washed with water (10 mL) and saturated brine (10 mL), dried and concentrated to give the product (950 mg, yield: 87%).

### Step 5: Synthesis of Intermediate tert-butyl (R)-3-((8-(2-methoxy-4-methylphenyl)pyrido[2,3-d]pyridazin-5-yl)amino)piperidine-1-carboxylate

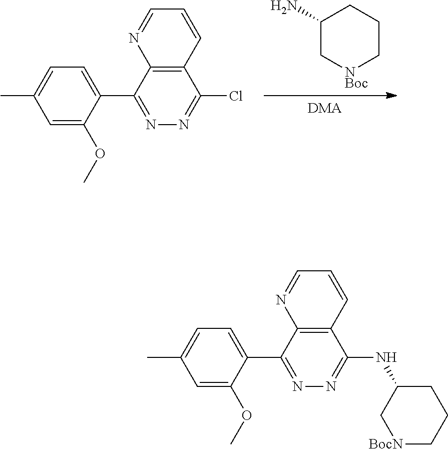

5-Chloro-8-(2-methoxy-4-methylphenyl)pyrido[2,3-d]pyridazine (950 mg, 3.325 mmol, 1.0 eq.) was dissolved in DMA (10 mL), followed by addition of (R)-1-tert-butoxycarbonyl-3-aminopiperidine (1.33 g, 6.65 mmol, 2.0 eq.). The mixture was refluxed at 120° C. for 64 h. After the reaction was completed as detected by TLC, the reaction solution was added dropwise into water (10 mL), and ethyl acetate (10 mL) was added for extraction. The organic phase was washed with water (10 mL) and saturated brine (10 mL), dried and concentrated, and the crude product was subjected to silica gel column chromatography (petroleum ether:ethyl acetate=1.5:1 to only ethyl acetate) to give the product (805 mg, yield: 54%).

### Step 6: Synthesis of Intermediate (R)-5-methyl-2-(5-((piperidin-3-yl)amino)pyrido[2,3-d]pyridazin-8-yl)phenol

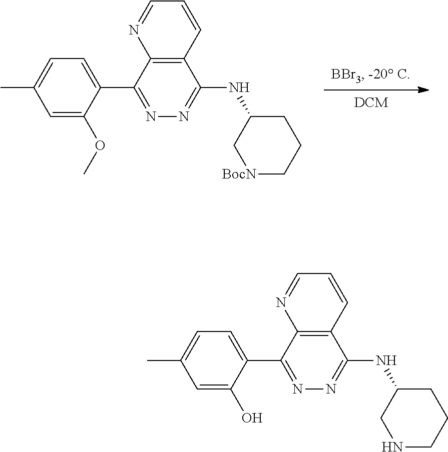

Tert-butyl (R)-3-((8-(2-methoxy-4-methylphenyl)pyrido[2,3-d]pyridazin-5-yl)amino)piperidine-1-carboxylate (805 mg, 1.79 mmol, 1.0 eq.) was dissolved in dichloromethane (5 mL), followed by dropwise addition of boron tribromide (1.35 g, 5.37 mmol, 3.0 eq.) at −20° C. The mixture was reacted at room temperature for 1 h. After the reaction was not completed as detected by LC-MS, the reaction solution was supplemented with boron tribromide (1.35 g, 5.37 mmol, 3.0 eq.). The resulting mixture was reacted at room temperature for 1 h. After the reaction was completed as detected by LC-MS, methanol (1 mL) and water (10 mL) were added dropwise and slowly to the reaction solution under an ice bath, followed by extraction with dichloromethane (10 mL). The organic phase was dried and concentrated to give the product (146 mg, yield: 24.3%).

### Step 7: Synthesis of compound (R)-5-methyl-2-(5-((1-methylpiperidin-3-yl)amino)pyrido[2,3-d]pyridazin-8-yl)phenol

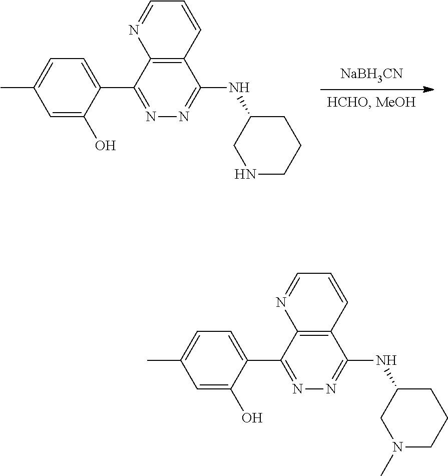

(R)-5-Methyl-2-(5-((piperidin-3-yl)amino)pyrido[2,3-d]pyridazin-8-yl)phenol (146 mg, 0.435 mmol, 1.0 eq.) was dissolved in methanol (2 mL), followed by addition of an aqueous formaldehyde solution (38.5%) (34 mg, 0.435 mmol, 1.0 eq.). The mixture was stirred for 5 min, followed by addition of sodium cyanoborohydride (30 mg, 0.479 mmol, 1.1 eq.). The resulting mixture was reacted at room temperature for 10 min. After the reaction was completed as detected by TLC, the reaction solution was concentrated, and the crude product was purified by preparative thin-layer chromatography (dichloromethane:methanol=7:1) to give the product (34 mg, yield: 22.3%).

¹H-NMR (300 MHz, DMSO-d6) δ (ppm): 11.92 (brs, 1H), 9.20-9.22 (m, 1H), 9.01 (s, 1H), 8.18-8.21 (d, 1H), 7.95-7.99 (m, 1H), 7.84 (s, 1H), 6.77 (s, 1H), 6.73 (s, 1H), 4.66 (s, 1H), 3.55 (s, 3H), 2.72 (s, 4H), 2.31 (s, 3H), 1.99-2.02 (d, 2H), 1.79-1.83 (d, 2H)

Molecular formula: C₂₀H₂₃N₅O Molecular weight: 349.44 LC-MS: 350.2 [M+H]⁺.

### Example 8: Synthesis of (R)-5-methyl-2-(8-((1-methylpiperidin-3-yl)amino)pyrido[2,3-d]pyridazin-5-yl)phenol (Compound 38)

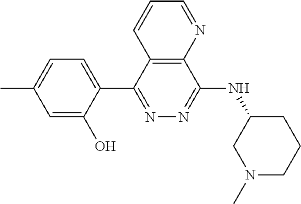 [SMILES: Cc1ccc(-c2nnc(N[C@@H]3CCCN(C)C3)c3ncccc23)c(O)c1]

### Step 1: Synthesis of Intermediate 3-(2-methoxy-4-methylbenzoyl)nicotinic acid

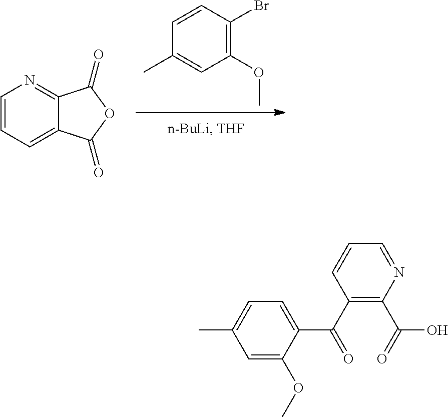

1-Bromo-2-methoxy-4-methylbenzene (14.15 g, 70.38 mmol, 1.05 eq.) was dissolved in anhydrous THE (100 mL), followed by dropwise addition of n-BuLi (33.8 mL, 80.48 mmol, 1.2 eq.) at −78° C. After the completion of dropwise addition, the mixture was reacted for 30 min. At −78° C., the reaction solution was added dropwise to a solution of 2,3-pyridine dicarboxylic anhydride (10 g, 67.07 mmol, 1.0 eq.) in THE (100 mL). After the completion of dropwise addition, the mixture was reacted at room temperature for 16 h. After the reaction was completed as detected by LC-MS, the reaction solution was poured into a mixed solution of ice water (50 mL) and 2 N HCl (50 mL), and ethyl acetate (200 mL) was added for extraction. The aqueous phase was extracted with dichloromethane (200 mL), and the organic phases were combined, dried and concentrated to give the crude product (26 g). The crude product was purified by silica gel column chromatography (petroleum ether: ethyl acetate=2:1, dichloromethane:methanol=10:1) to give the product (1 g, yield: 5.5%).

### Step 2: Synthesis of Intermediate methyl 3-(2-methoxy-4-methylbenzoyl)nicotinate

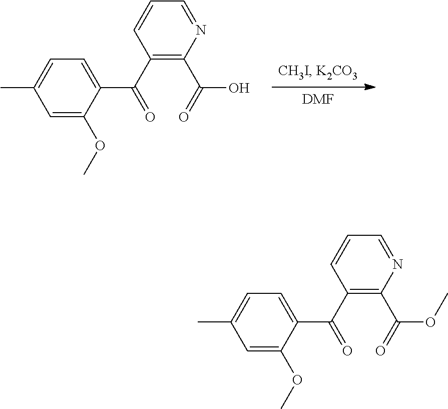

The 3-(2-methoxy-4-methylbenzoyl)nicotinic acid (1 g, 3.69 mmol, 1.0 eq.) obtained in the previous step was dissolved in DMF (15 mL), followed by addition of potassium carbonate (766 mg, 5.54 mmol, 1.5 eq.) and iodomethane (629 mg, 4.43 mmol, 1.2 eq.). The mixture was reacted at room temperature for 16 h. After the reaction was completed as detected by TLC, the reaction solution was poured into water (20 mL), and EA (20 mL) was added for extraction. The organic phase was washed with water (20 mL) and saturated brine (20 mL), dried over anhydrous sodium sulfate and concentrated to give the product (456 mg, yield: 43.4%).

### Step 3: Synthesis of Intermediate 5-(2-methoxy-4-methylphenyl)pyrido[2,3-d]pyridazin-8-ol

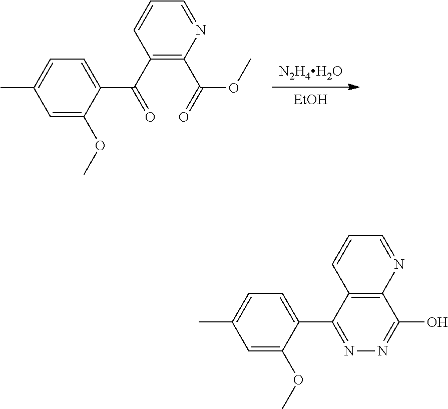

Methyl 3-(2-methoxy-4-methylbenzoyl)nicotinate (456 mg, 1.6 mmol, 1.0 eq.) obtained in the previous step was dissolved in ethanol (10 mL), followed by addition of 85% hydrazine hydrate (142 mg, 2.4 mmol, 1.5 eq.). The mixture was heated to 85° C. and reacted for 2 h. After the reaction was completed as detected by TLC, the reaction solution was concentrated. Water (10 mL) was added and dichloromethane (10 mL) was added for extraction. The organic phase was dried and concentrated to give the product (230 mg, yield: 53.8%).

### Step 4: Synthesis of Intermediate 8-chloro-5-(2-methoxy-4-methylphenyl) pyrido[2,3-d]pyridazine

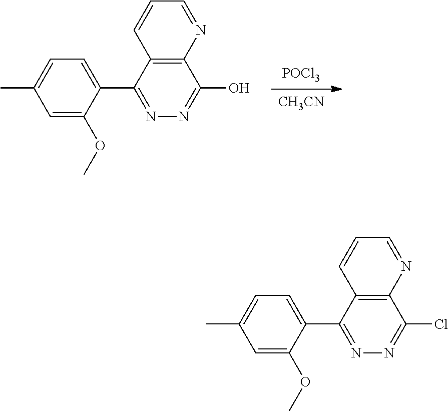

Acetonitrile (3 mL) was added to 5-(2-methoxy-4-methylphenyl)pyrido[2,3-d]pyridazin-8-ol (230 mg, 0.86 mmol, 1.0 eq.), followed by addition of phosphorus oxychloride (264 mg, 1.72 mmol, 2.0 eq.). The mixture was reacted at 85° C. for 6 h. After the reaction was completed as detected by TLC, the reaction solution was concentrated. The concentrated solution was poured into ice water (10 mL), and dichloromethane (10 mL) was added for extraction. The organic phase was washed with water (10 mL) and saturated brine (10 mL), dried and concentrated to give the product (112 mg, yield: 45.7%).

### Step 5: Synthesis of Intermediate tert-butyl (R)-3-((5-(2-methoxy-4-methylphenyl)pyrido[2,3-d]pyridazin-8-yl)amino)piperidine-1-carboxylate

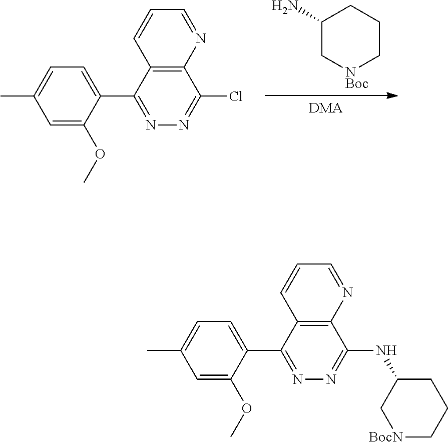

8-Chloro-5-(2-methoxy-4-methylphenyl)pyrido[2,3-d]pyridazine (112 mg, 0.392 mmol, 1.0 eq.) was dissolved in DMA (2 mL), followed by addition of (R)-1-tert-butoxycarbonyl-3-aminopiperidine (157 mg, 0.784 mmol, 2.0 eq.). The mixture was refluxed at 120° C. for 64 h. After the reaction was completed as detected by TLC, the reaction solution was added dropwise into water (5 mL), and ethyl acetate (5 mL) was added for extraction. The organic phase was washed with water (5 mL) and saturated brine (5 mL), dried and concentrated to give the crude product (280 mg).

### Step 6: Synthesis of Intermediate (R)-8-methyl-2-(5-((piperidin-3-yl)amino)pyrido[2,3-d]pyridazin-5-yl)phenol

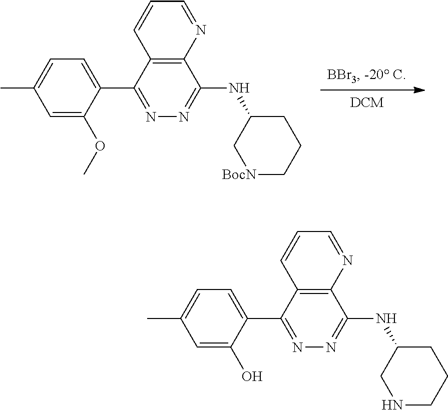

Tert-butyl (R)-3-((5-(2-methoxy-4-methylphenyl)pyrido[2,3-d]pyridazin-8-yl)amino)piperidine-1-carboxylate (280 mg, crude product, 0.392 mmol, 1.0 eq.) was dissolved in dichloromethane (2 mL), followed by dropwise addition of boron tribromide (295 mg, 1.176 mmol, 3.0 eq.) at −20° C. The mixture was reacted at room temperature for 1 h. After the reaction was not completed as detected by LC-MS, the reaction solution was supplemented with boron tribromide (295 mg, 1.176 mmol, 3.0 eq.). The resulting mixture was reacted at room temperature for 1 h. After the reaction was completed as detected by LC-MS, methanol (5 mL) and water (10 mL) were added dropwise and slowly to the reaction solution under an ice bath. Dichloromethane (10 mL) was added for extraction, and the product was in the aqueous phase. The pH of the aqueous phase was adjusted to be neutral with a saturated aqueous sodium bicarbonate solution, and dichloromethane (10 mL) was added for extraction. The organic phase was dried and concentrated to give the product (70 mg, two-step yield: 53%).

### Step 7: Synthesis of compound (R)-5-methyl-2-(8-((1-methylpiperidin-3-yl)amino)pyrido[2,3-d]pyridazin-5-yl)phenol

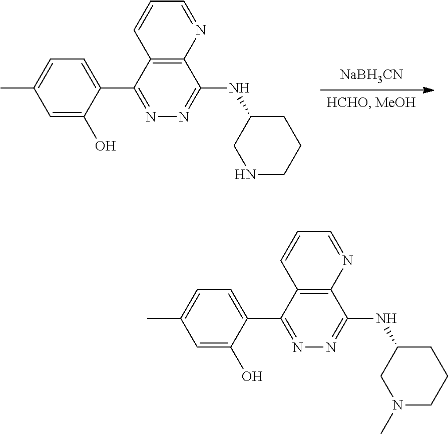

(R)-8-Methyl-2-(5-((piperidin-3-yl)amino)pyrido[2,3-d]pyridazin-5-yl)phenol (70 mg, 0.209 mmol, 1.0 eq.) was dissolved in methanol (1 mL), followed by addition of an aqueous formaldehyde solution (38.5%) (16.3 mg, 0.209 mmol, 1.0 eq.). The mixture was stirred for 5 min, followed by addition of sodium cyanoborohydride (14.5 mg, 0.230 mmol, 1.1 eq.). The resulting mixture was reacted at room temperature for 10 min. After the reaction was completed as detected by TLC, the reaction solution was concentrated, and the crude product was purified by preparative thin-layer chromatography (dichloromethane:methanol=7:1) to give the product (17 mg, yield: 23.2%).

¹H-NMR (300 MHz, DMSO-d6) δ (ppm): 9.71 (s, 1H), 9.11-9.13 (m, 1H), 7.85-7.98 (m, 2H), 7.54 (s, 1H), 7.22-7.24 (d, 1H), 6.79-6.83 (m, 2H), 4.59 (s, 1H), 2.92-3.04 (m, 2H), 2.74 (m, 2H), 2.59 (s, 3H), 2.34 (s, 3H), 1.78-1.92 (m, 4H).

Molecular formula: C₂₀H₂₃N₅O Molecular weight: 349.44 LC-MS: 350.2 [M+H]⁺.

### Example 9: Synthesis of (R)-4-(2-amino-4-methylphenyl)-N-(1-methylpiperidin-3-yl)phthalazin-1-amine (Compound 39)

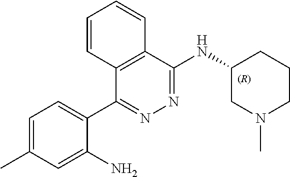

### Step 1: Synthesis of 2-bromo-5-methylaniline

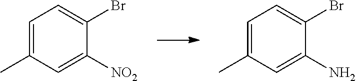

1-Bromo-4-methyl-2-nitrobenzene (20.0 g, 92.57 mmol, 1.0 eq.) was added into ethanol (200.0 mL), followed by addition of a saturated aqueous ammonium chloride solution (30.0 mL) and reduced iron powder (51.8 g, 925.7 mmol, 10.0 eq.). The mixture was stirred at 80° C. for 4 h. After the reaction was completed as monitored by TLC, the system was cooled to room temperature and filtered under vacuum. The filtrate was concentrated under reduced pressure, and dichloromethane (200.0 mL) was added, followed by washing with water (100.0 mL). The organic phase was dried over anhydrous sodium sulfate and filtered, and the filtrate was concentrated under reduced pressure. The crude product was purified by silica gel column chromatography (specification of silica gel: 100-200 mesh, petroleum ether:ethyl acetate=20:1) to give the product (16.0 g, yield: 92.9%).

### Step 2: Synthesis of 5-methyl-2-(4,4,5,5-tetramethyl-1,3,2-dioxaborolan-2-yl)aniline

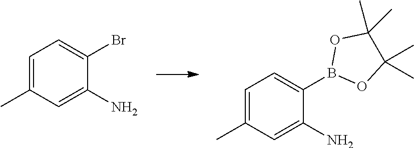

2-Bromo-5-methylaniline (16.0 g, 85.99 mmol, 1.0 eq.), bis(pinacolato)diboron (32.7 g, 128.99 mmol, 1.5 eq.), potassium acetate (16.8 g, 171.98 mmol, 2.0 eq.) and Pd(dppf)Cl₂ (3.1 g, 4.29 mmol, 0.05 eq.) were added to 1,4-dioxane (200.0 mL), and the mixture was reacted at 100° C. for 3 h in nitrogen atmosphere. After the reaction was completed as monitored by TLC, the system was concentrated, and the crude product was purified by silica gel column chromatography (specification of silica gel: 100-200 mesh, petroleum ether:ethyl acetate=50:1 to 30:1) to give the product (15.0 g, yield: 75.0%).

### Step 3: Synthesis of tert-butyl (5-methyl-2-(4,4,5,5-tetramethyl-1,3,2-dioxaborolan-2-yl)phenyl)carbamate

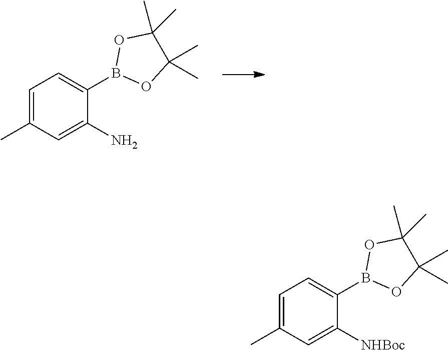

5-Methyl-2-(4,4,5,5-tetramethyl-1,3,2-dioxaborolan-2-yl)aniline (10.0 g, 42.89 mmol, 1.0 eq.), di-tert-butyl dicarbonate (18.7 g, 85.79 mmol, 2.0 eq.) and 4-dimethylaminopyridine (7.85 g, 64.33 mmol, 1.5 eq.) were added to dichloromethane (150.0 mL), and the mixture was reacted at room temperature for 3 h. After the reaction was completed as monitored by TLC, dichloromethane (300.0 mL) was added to the system, followed by washing with a saturated aqueous ammonium chloride solution (200.0 mL). The organic phase was dried over anhydrous sodium sulfate and filtered, and the filtrate was concentrated under reduced pressure. The crude product was purified by silica gel column chromatography (specification of silica gel: 100-200 mesh, petroleum ether:ethyl acetate=100:1 to 80:1 to 60:1) to give the product (8.0 g, yield: 57.1%).

### Step 4: Synthesis of tert-butyl (R)-3-((4-chlorophthalazin-1-yl)amino) piperidine-1-carboxylate

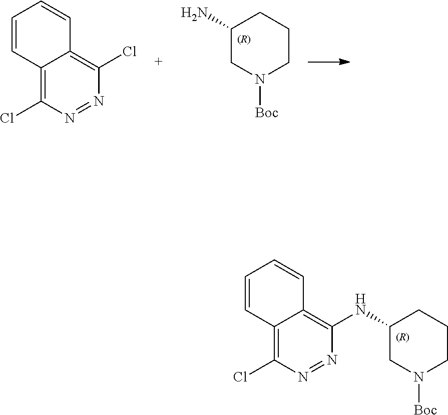

1,4-Dichlorophthalazine (3.67 g, 18.47 mmol, 1.0 eq.) and tert-butyl (R)-3-aminopiperidine-1-carboxylate (7.4 g, 36.94 mmol, 2.0 eq.) were added to N,N-dimethylacetamide (15.0 mL), and the mixture was stirred at 120° C. for 12 h. After the reaction was completed as monitored by TLC, ethyl acetate (200.0 mL) was added to the system, followed by washing with water (100.0 mL×3). The organic phase was dried over anhydrous sodium sulfate and filtered, and the filtrate was concentrated under reduced pressure. The crude product was purified by silica gel column chromatography (specification of silica gel: 100-200 mesh, petroleum ether:ethyl acetate=10:1 to 2:1) to give the product (1.5 g, yield: 22.4%).

### Step 5: Synthesis of (R)-4-chloro-N-(piperidin-3-yl)phthalazin-1-amine

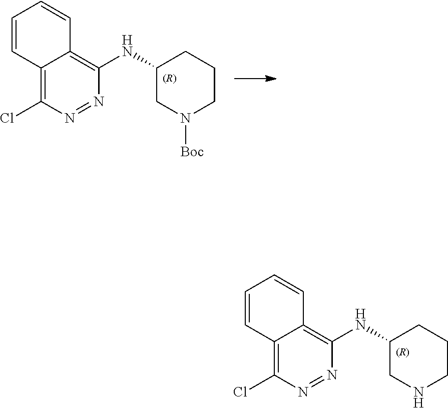

Tert-butyl (R)-3-((4-chlorophthalazin-1-yl)amino)piperidine-1-carboxylate (1.5 g, 4.13 mmol, 1.0 eq.) and trifluoroacetic acid (6.0 mL) were added to dichloromethane (6.0 mL), and the mixture was stirred at room temperature for 2 h. After the reaction was completed as monitored by TLC, the system was back-extracted with water (100.0 mL), and the pH of the aqueous phase was adjusted to 8-9, followed by addition of dichloromethane (100.0 mL×3) for extraction. The organic phases were combined, dried over anhydrous sodium sulfate and filtered, and the filtrate was concentrated under reduced pressure to give the product (1.0 g, yield: 92.1%).

### Step 6: Synthesis of (R)-4-chloro-N-(1-methylpiperidin-3-yl)phthalazin-1-amine

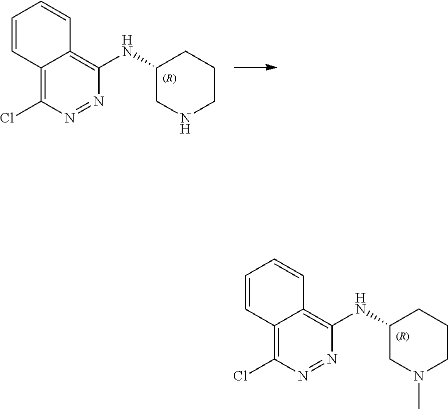

(R)-4-chloro-N-(piperidin-3-yl)phthalazin-1-amine (1.0 g, 3.80 mmol, 1.0 eq.), dichloromethane (3.0 mL) and an aqueous formaldehyde solution (37%) (308.9 mg, 3.80 mmol, 1.0 eq.) were added to methanol (10.0 mL), and the mixture was stirred at room temperature for 1 h, followed by addition of sodium cyanoborohydride (286.5 mg, 4.56 mmol, 1.2 eq.). The resulting mixture was reacted at room temperature for 1 h. After the reaction was completed as monitored by TLC, the system was concentrated under reduced pressure, followed by addition of a saturated aqueous sodium bicarbonate solution (100.0 mL). The system was stirred for 30 min and extracted with dichloromethane (100.0 mL). The organic phase was dried over anhydrous sodium sulfate and filtered, and the filtrate was concentrated under reduced pressure. The crude product was purified by silica gel column chromatography (specification of silica gel: 100-200 mesh, dichloromethane:methanol=50:1 to 30:1 to 10:1) to give the product (820.0 mg, yield: 78.0%).

### Step 7: Synthesis of tert-butyl (R)-(5-methyl-2-(4-((1-methylpiperidin-3-yl)amino)phthalazin-1-yl)phenyl)carbamate

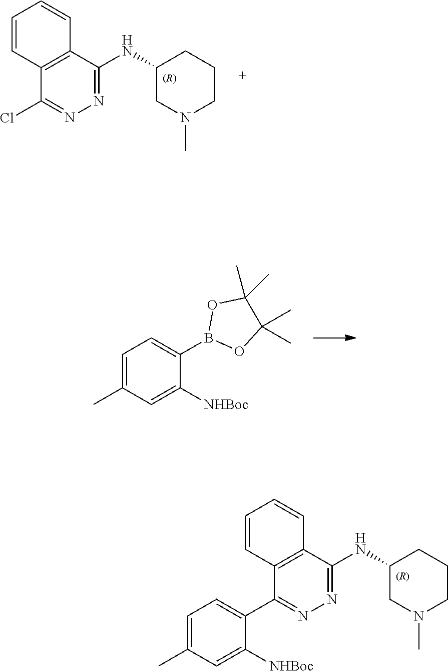

(R)-4-chloro-N-(1-methylpiperidin-3-yl)phthalazin-1-amine (274.0 mg, 0.98 mmol, 1.0 eq.), tert-butyl (5-methyl-2-(4,4,5,5-tetramethyl-1,3,2-dioxaborolan-2-yl)phenyl)carbamate (489.8 mg, 1.47 mmol, 1.5 eq.), sodium bicarbonate (164.6 mg, 1.96 mmol, 2.0 eq.), water (3.0 mL) and Pd(PPh₃)₄ (113.2 mg, 0.098 mmol, 0.1 eq.) were added to 1,4-dioxane (8.0 mL), and the mixture was reacted at 110° C. for 4 h in nitrogen atmosphere. After the reaction was completed as monitored by TLC, the system was cooled to room temperature and filtered, and ethyl acetate (100.0 mL) was added to the filtrate, followed by washing with water (100.0 mL). The organic phase was dried over anhydrous sodium sulfate and filtered, and the filtrate was concentrated under reduced pressure. The crude product was purified by preparative thin-layer chromatography (dichloromethane:methanol=10:1) to give the product (201.0 mg, yield: 45.8%).

### Step 8: Synthesis of (R)-4-(2-amino-4-methylphenyl)-N-(1-methylpiperidin-3-yl)phthalazin-1-amine

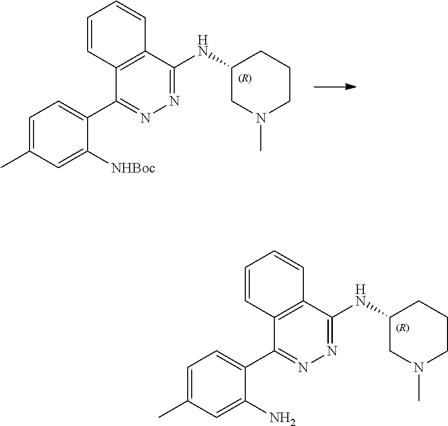

Tert-butyl (R)-(5-methyl-2-(4-((1-methylpiperidin-3-yl)amino)phthalazin-1-yl)phenyl)carbamate (201.0 mg, 0.44 mmol, 1.0 eq.) and trifluoroacetic acid (3.0 mL) were added to dichloromethane (3.0 mL), and the mixture was reacted at room temperature for 1 h. After the reaction was completed as monitored by TLC, the system was back-extracted with water (50.0 mL), the pH of the aqueous phase was adjusted to 8-9, and dichloromethane (50.0 mL×2) was added for extraction. The organic phase was dried over anhydrous sodium sulfate and filtered, and the filtrate was concentrated under reduced pressure to give the product (120.0 mg, yield: 76.9%).

¹HNMR (300 MHz, DMSO-d6) δ (ppm): 8.37-8.40 (d, J=9 Hz, 1H), 7.82-7.87 (m, 2H), 7.55-7.58 (d, J=9 Hz, 1H), 7.02-7.05 (d, J=9 Hz, 1H), 6.93-6.96 (d, J=9 Hz, 1H), 6.65 (s, 1H), 6.50-6.53 (d, J=9 Hz, 1H), 4.89 (s, 2H), 4.41-4.43 (d, J=6 Hz, 1H), 3.08-3.10 (d, J=6 Hz, 1H), 2.70-2.73 (d, J=9 Hz, 1H), 2.27 (s, 3H), 2.22 (s, 3H), 1.40-1.99 (m, 6H).

Molecular formula: C₂₁H₂₅N₅ Molecular weight: 347.47 LC-MS (Pos, m/z)=348.25[M+H]⁺.

### Example 10: Synthesis of (R)-4-(4-methyl-2-(methylamino)phenyl)-N-(1-methylpiperidin-3-yl)phthalazin-1-amine (Compound 40)

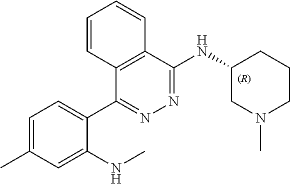

### Step 1: Synthesis of 2-bromo-N,5-dimethylaniline

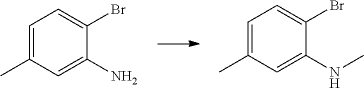

2-Bromo-5-methylaniline (5.0 g, 26.87 mmol, 1.0 eq.) was added to tetrahydrofuran (20.0 mL), and the mixture was cooled to −70° C. in nitrogen atmosphere, followed by dropwise addition of a solution of n-butyllithium in n-hexane (2.5 M) (10.7 mL, 26.87 mmol, 1.0 eq.). The resulting mixture was reacted for 1 h and then warmed to room temperature, followed by dropwise addition of iodomethane (3.8 g, 26.87 mmol, 1.0 eq.). The resulting mixture was reacted at room temperature for 3 h. After the reaction was completed as monitored by TLC, ethyl acetate (200.0 mL) was added to the system, followed by washing with a saturated aqueous ammonium chloride solution (100.0 mL). The organic phase was dried over anhydrous sodium sulfate and filtered, and the filtrate was concentrated under reduced pressure. The crude product was purified by silica gel column chromatography (specification of silica gel: 100-200 mesh, petroleum ether:ethyl acetate=100:1) to give the product (4.7 g, yield: 87.4%).

### Step 2: Synthesis of N,5-dimethyl-2-(4,4,5,5-tetramethyl-1,3,2-dioxaborolan-2-yl)aniline

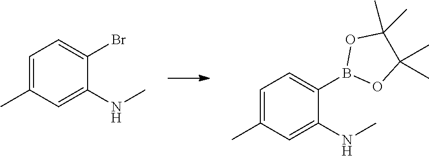

2-Bromo-N,5-dimethylaniline (1.0 g, 4.99 mmol, 1.0 eq.), bis(pinacolato)diboron (1.9 g, 7.49 mmol, 1.5 eq.), potassium acetate (979.4 mg, 9.98 mmol, 2.0 eq.) and Pd(PPh₃)₄ (288.3 mg, 0.24 mmol, 0.05 eq.) were added to 1,4-dioxane (10.0 mL), followed by purge with nitrogen for 5 min. The mixture was reacted at 100° C. for 12 h. After the reaction was completed as monitored by TLC, the reaction solution was concentrated and purified by silica gel column chromatography (specification of silica gel: 100-200 mesh, petroleum ether:ethyl acetate=1:1) to give the product (800.0 mg, yield: 64.3%).

### Step 3: Synthesis of (R)-4-(4-methyl-2-(methylamino)phenyl)-N-(1-methylpiperidin-3-yl)phthalazin-1-amine

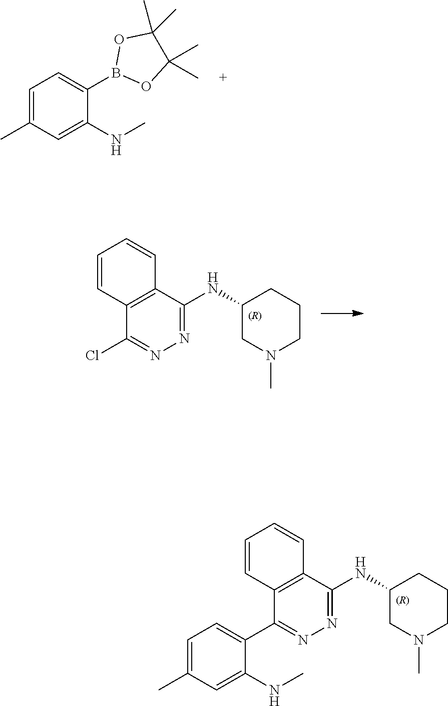

(R)-4-chloro-N-(1-methylpiperidin-3-yl)phthalazin-1-amine (160.0 mg, 0.57 mmol, 1.0 eq.), N,5-dimethyl-2-(4,4,5,5-tetramethyl-1,3,2-dioxaborolan-2-yl)aniline (211.3 mg, 0.85 mmol, 1.5 eq.), sodium bicarbonate (95.7 mg, 1.14 mmol, 2.0 eq.), water (2.0 mL) and Pd(PPh₃)₄ (32.9 mg, 0.02 mmol, 0.05 eq.) were added to 1,4-dioxane (6.0 mL), and the mixture was reacted at 110° C. for 12 h in nitrogen atmosphere. After the reaction was completed as monitored by TLC, the system was cooled to room temperature and filtered, and ethyl acetate (100.0 mL) was added to the filtrate, followed by washing with water (100.0 mL). The organic phase was dried over anhydrous sodium sulfate and filtered, and the filtrate was concentrated under reduced pressure. The crude product was purified by preparative thin-layer chromatography (dichloromethane:methanol=8:1) to give the product (70.0 mg, yield: 33.9%).

¹HNMR (300 MHz, DMSO-d6) δ (ppm): 8.42-8.45 (d, J=9 Hz, 1H), 7.75-7.88 (m, 2H), 7.49-7.51 (d, J=6 Hz, 1H), 7.24 (s, 1H), 6.93-6.96 (d, J=9 Hz, 1H), 6.53-6.56 (m, 2H), 5.02-5.03 (d, J=3 Hz, 1H), 4.56 (s, 1H), 2.95-2.98 (d, J=9 Hz, 1H), 2.62-2.63 (d, J=3 Hz, 3H), 2.46 (s, 3H), 2.34 (s, 3H), 1.60-2.28 (m, 6H).

Molecular formula: C₂₂H₂₇N₅ Molecular weight: 361.49 LC-MS (Pos, m/z)=362.26[M+H]⁺.

### Example 11: Synthesis of (R)-5-methyl-2-(4-(methyl(1-methylpiperidin-3-yl)amino)phthalazin-1-yl)phenol (Compound 41)

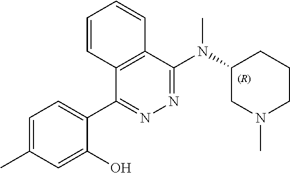

### Step 1: Synthesis of tert-butyl (R)-3-((4-(2-hydroxy-4-methylphenyl)phthalazin-1-yl)(methyl)amino)piperidine-1-carboxylate

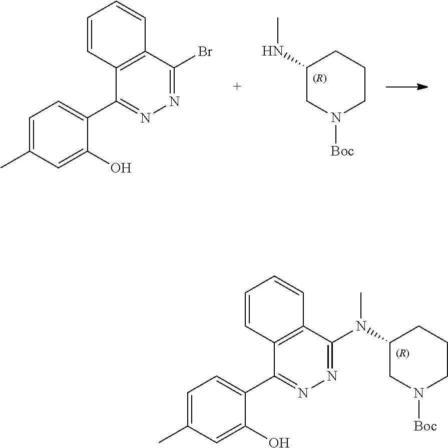

2-(4-Bromophthalazin-1-yl)-5-methylphenol (300.0 mg, 0.95 mmol, 1.0 eq.) and tert-butyl (R)-3-(methylamino)piperidine-1-carboxylate (407.9 mg, 1.90 mmol, 2.0 eq.) were added to N,N-dimethylacetamide (10.0 mL), and the mixture was stirred at 120° C. for 12 h. After the reaction was completed as monitored by TLC, the system was cooled to room temperature, and ethyl acetate (100.0 mL) was added, followed by washing with water (100.0 mL). The organic phase was dried over anhydrous sodium sulfate and filtered, and the filtrate was concentrated under reduced pressure. The crude product was purified by silica gel column chromatography (specification of silica gel: 100-200 mesh, petroleum ether:ethyl acetate=20:1 to 6:1) to give the product (160.0 mg, yield: 37.5%).

### Step 2: Synthesis of (R)-5-methyl-2-(4-(methyl(piperidin-3-yl)amino) phthalazin-1-yl)phenol

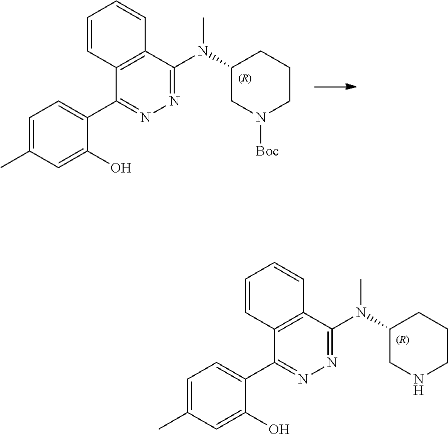

Tert-butyl (R)-3-((4-(2-hydroxy-4-methylphenyl)phthalazin-1-yl)(methyl) amino)piperidine-1-carboxylate (160.0 mg, 0.35 mmol, 1.0 eq.) and trifluoroacetic acid (5.0 mL) were added to dichloromethane (5.0 mL), and the mixture was stirred at room temperature for 2 h. After the reaction was completed as monitored by TLC, the system was back-extracted with water (80.0 mL), the pH of the aqueous phase was adjusted to 8-9, and dichloromethane (100.0 mL×2) was added for extraction. The organic phases were combined, dried over anhydrous sodium sulfate and filtered, and the filtrate was concentrated under reduced pressure to give the product (98.0 mg, yield: 80.3%).

### Step 3: Synthesis of (R)-5-methyl-2-(4-(methyl(1-methylpiperidin-3-yl)amino) phthalazin-1-yl)phenol

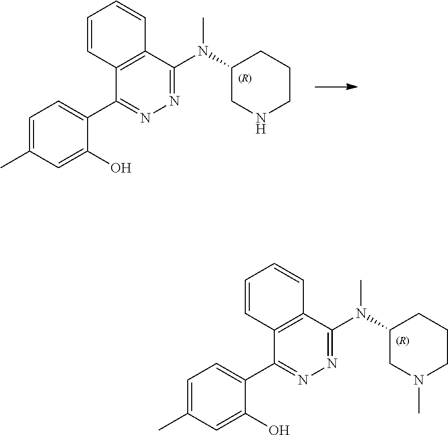

(R)-5-methyl-2-(4-(methyl(piperidin-3-yl)amino)phthalazin-1-yl)phenol (98.0 mg, 0.28 mmol, 1.0 eq.) and an aqueous formaldehyde solution (37%) (22.8 mg, 0.28 mmol, 1.0 eq.) were added to methanol (5.0 mL), and the mixture was stirred at room temperature for 1 h, followed by addition of sodium cyanoborohydride (19.3 mg, 0.30 mmol, 1.1 eq.). The resulting mixture was reacted at room temperature for 2 h. After the reaction was completed as monitored by TLC, a saturated aqueous sodium bicarbonate solution (50.0 mL) was added to the system, and the mixture was stirred for 30 min, followed by extraction with dichloromethane (100.0 mL). The organic phase was dried over anhydrous sodium sulfate and filtered, and the filtrate was concentrated under reduced pressure. The crude product was purified by preparative thin-layer chromatography (dichloromethane:methanol=10:1) to give the product (30.0 mg, yield: 29.5%).

¹HNMR (300 MHz, DMSO-d6) δ (ppm): 9.63 (s, 1H), 8.36-8.48 (m, 1H), 8.11-8.19 (m, 1H), 7.89-7.94 (m, 1H), 7.79-7.85 (m, 1H), 7.54-7.57 (d, J=9 Hz, 1H), 7.03 (s, 1H), 6.85-6.88 (d, J=9 Hz, 1H), 4.26 (s, 1H), 3.46-3.49 (m, 1H), 3.19-3.28 (m, 4H), 2.66 (s, 3H), 2.08 (m, 1H), 2.45 (s, 3H), 1.96-2.06 (m, 5H).

Molecular formula: C₂₂H₂₆N₄O Molecular weight: 362.48 LC-MS (Pos, m/z)=363.33[M+H]⁺.

### Example 12: Synthesis of (R)-3-hydroxy-4-(4-((1-methylpiperidin-3-yl)amino)phthalazin-1-yl)benzonitrile (Compound 42)

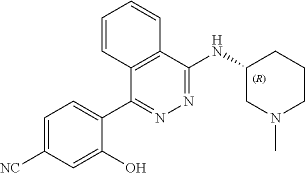

### Step 1: Synthesis of 3-methoxy-4-(4,4,5,5-tetramethyl-1,3,2-dioxaborolan-2-yl)benzonitrile

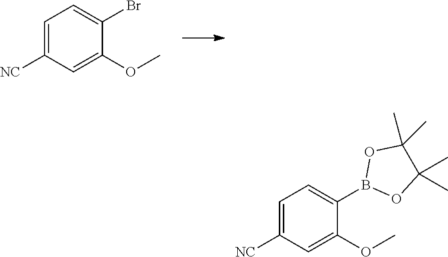 [SMILES: COc1cc(C#N)ccc1Br ; COc1cc(C#N)ccc1B1OC(C)(C)C(C)(C)O1]

4-Bromo-3-methoxybenzonitrile (5.0 g, 23.50 mmol, 1.0 eq.), bis(pinacolato)diboron (11.97 g, 47.15 mmol, 2.0 eq.), potassium acetate (6.94 g, 70.73 mmol, 3.0 eq.) and Pd(PPh₃)₄ (1.36 g, 1.17 mmol, 0.05 eq.) were added to 1,4-dioxane (100.0 mL), and the mixture was reacted at 100° C. for 12 h in nitrogen atmosphere. After the reaction was completed as monitored by TLC, the system was directly concentrated, and the crude product was purified by silica gel column chromatography (specification of silica gel: 100-200 mesh, petroleum ether:ethyl acetate=20:1) to give the product (5.1 g, yield: 85.0%).

### Step 2: Synthesis of (R)-3-methoxy-4-(4-((1-methylpiperidin-3-yl)amino) phthalazin-1-yl)benzonitrile

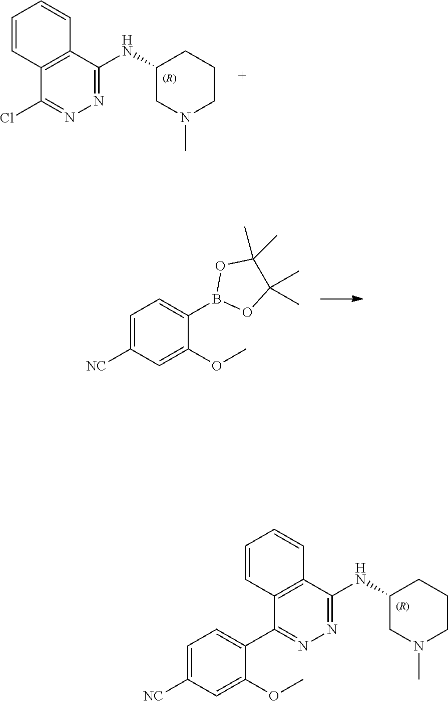

(R)-4-chloro-N-(1-methylpiperidin-3-yl)phthalazin-1-amine (154.0 mg, 0.55 mmol, 1.0 eq.), 3-methoxy-4-(4,4,5,5-tetramethyl-1,3,2-dioxaborolan-2-yl)benzonitrile (288.3 mg, 1.11 mmol, 2.0 eq.), sodium bicarbonate (140.1 mg, 1.66 mmol, 3.0 eq.), water (1.0 mL) and Pd(PPh₃)₄ (32.1 mg, 0.027 mmol, 0.05 eq.) were added to 1,4-dioxane (3.0 mL), and the mixture was reacted at 110° C. for 3 h in nitrogen atmosphere. After the reaction was completed as monitored by TLC, the system was cooled to room temperature and filtered, and ethyl acetate (100.0 mL) was added to the filtrate, followed by washing with water (100.0 mL). The organic phase was dried over anhydrous sodium sulfate and filtered, and the filtrate was concentrated under reduced pressure. The crude product was purified by silica gel column chromatography (specification of silica gel: 100-200 mesh, dichloromethane:methanol=50:1 to 10:1) to give the product (165.0 mg, yield: 79.7%).

### Step 3: Synthesis of (R)-5-methyl-2-(4-((1-methylpiperidin-3-yl)amino) phthalazin-1-yl)phenol

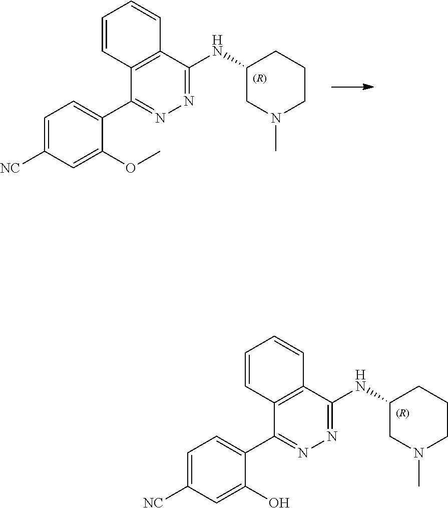

(R)-3-methoxy-4-(4-((1-methylpiperidin-3-yl)amino)phthalazin-1-yl)benzonitrile (165.0 mg, 0.44 mmol, 1.0 eq.) was added to dichloromethane (4.0 mL), and the mixture was cooled to −20° C. in nitrogen atmosphere, followed by dropwise addition of boron tribromide (330.6 mg, 1.32 mmol, 3.0 eq.). The resulting mixture was gradually warmed to room temperature and reacted for 2 h. After the reaction was completed as monitored by TLC, methanol (5.0 mL) was added dropwise and slowly to the system to quench the reaction, and the mixture was concentrated under reduced pressure, followed by slow addition of a saturated aqueous sodium bicarbonate solution (50.0 mL). The resulting mixture was stirred for 30 min, and the pH was adjusted to 7-8, followed by extraction with dichloromethane (50.0 mL). The organic phase was dried over anhydrous sodium sulfate and filtered, and the filtrate was concentrated under reduced pressure. The crude product was purified by preparative thin-layer chromatography (dichloromethane:methanol=5:1) to give the product (60.0 mg, yield 37.9%).

¹HNMR (300 MHz, DMSO-d6) δ (ppm): 10.55 (s, 1H), 8.49 (s, 1H), 7.79-7.92 (m, 2H), 7.37-7.50 (m, 5H), 4.64 (s, 1H), 3.12-3.16 (m, 2H), 2.63-2.68 (m, 4H), 1.75-2.01 (m, 5H).

Molecular formula: C₂₁H₂₁N₅O Molecular weight: 359.43 LC-MS (Pos, m/z)=360.22[M+H]⁺.

### Example 13: Synthesis of (R)-2-(8-fluoro-4-((1-methylpiperidin-3-yl)amino)phthalazin-1-yl)-5-methylphenol (Compound 6) and (R)-2-(5-fluoro-4-((1-methylpiperidin-3-yl)amino)phthalazin-1-yl)-5-methylphenol (Compound 9)

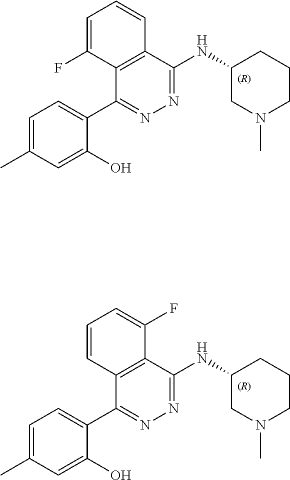

### Step 1: Synthesis of 3-fluoro-2-(2-methoxy-4-methylbenzoyl)benzoic acid and 2-fluoro-6-(2-methoxy-4-methylbenzoyl)benzoic acid

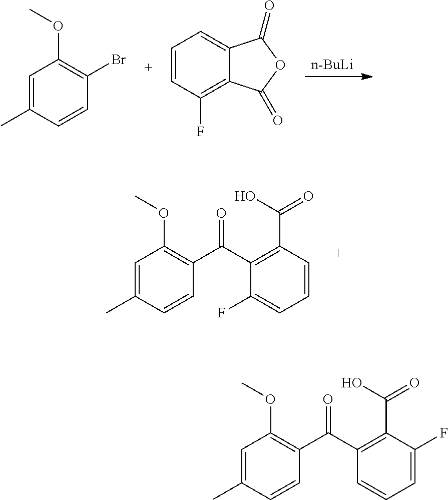

1-Bromo-2-methoxy-4-methylbenzene (6.66 g, 30.10 mmol, 1.1 eq.) was dissolved in THE (50 mL), and the mixture was cooled to −50° C., followed by dropwise and slow addition of a solution of n-butyllithium in n-hexane (2.5 mol/L, 13.2 mL, 30.10 mmol, 1.1 eq.). The reaction solution was stirred for 30 min and then added dropwise and slowly to a solution of 4-fluoroisobenzofuran-1,3-dione (5 g, 30.10 mmol, 1.0 eq.) in THE (50 mL) at −60° C., and the resulting mixture was reacted at −60° C. in nitrogen atmosphere. After the completion of dropwise addition, the reaction solution was naturally warmed to room temperature, reacted for 0.5 h and then poured into a dilute hydrochloric acid solution (100 mL), followed by addition of EA (100 mL×2) for extraction. The organic phase was dried and concentrated to give a mixture (8.67 g, yield: 100%) of 3-fluoro-2-(2-methoxy-4-methylbenzoyl)benzoic acid and 2-fluoro-6-(2-methoxy-4-methylbenzoyl)benzoic acid.

### Step 2: Synthesis of 5-fluoro-4-(2-methoxy-4-methylphenyl)phthalazin-1(2H)-one and 8-fluoro-4-(2-methoxy-4-methylphenyl)phthalazin-1(2H)-one

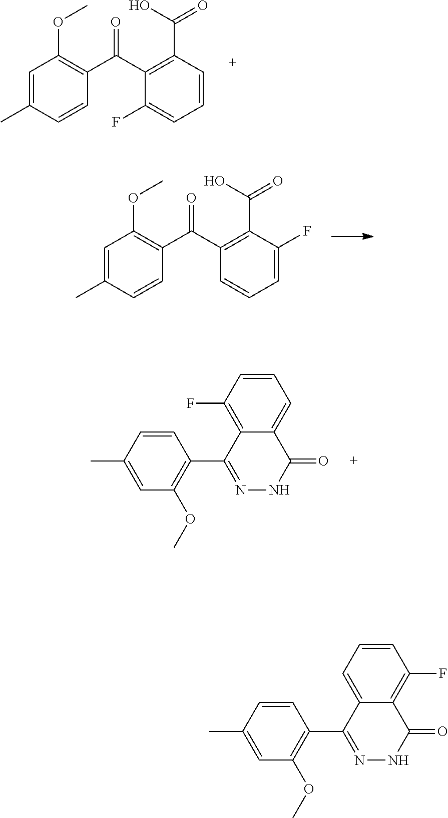 [SMILES: COc1cc(C)ccc1C(=O)c1c(F)cccc1C(=O)O ; COc1cc(C)ccc1C(=O)c1cccc(F)c1C(=O)O ; COc1cc(C)ccc1-c1n[nH]c(=O)c2cccc(F)c12 ; COc1cc(C)ccc1-c1n[nH]c(=O)c2c(F)cccc12]

The mixture (8.67 g, 30.10 mmol, 1.0 eq.) of 3-fluoro-2-(2-methoxy-4-methylbenzoyl)benzoic acid and 2-fluoro-6-(2-methoxy-4-methylbenzoyl)benzoic acid and 85% hydrazine hydrate (2.66 g, 45.15 mmol, 1.5 eq.) were dissolved in ethanol (100 mL), and the mixture was reacted at 80° C. for 3 h. After the generation of the target product as detected by LC-MS, the reaction solution was concentrated under reduced pressure, and the crude product was purified by column chromatography (PE:EA=3:1) to give a mixture (1.2 g, yield: 14.0%) of 5-fluoro-4-(2-methoxy-4-methylphenyl)phthalazin-1(2H)-one and 8-fluoro-4-(2-methoxy-4-methylphenyl)phthalazin-1(2H)-one.

### Step 3: Synthesis of 1-chloro-5-fluoro-4-(2-methoxy-4-methylphenyl phthalazine and 4-chloro-5-fluoro-1-(2-methoxy-4-methylphenyl)phthalazine

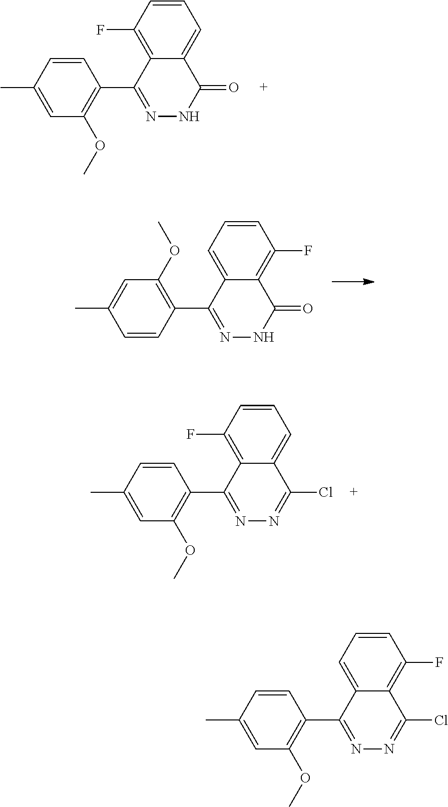 [SMILES: COc1cc(C)ccc1-c1n[nH]c(=O)c2cccc(F)c12 ; COc1cc(C)ccc1-c1n[nH]c(=O)c2c(F)cccc12 ; COc1cc(C)ccc1-c1nnc(Cl)c2cccc(F)c12 ; COc1cc(C)ccc1-c1nnc(Cl)c2c(F)cccc12]

5-Fluoro-4-(2-methoxy-4-methylphenyl)phthalazin-1(2H)-one, 8-fluoro-4-(2-methoxy-4-methylphenyl)phthalazin-1(2H)-one (1.2 g, 4.22 mmol, 1.0 eq.) and phosphorus oxychloride (1.29 g, 8.44 mmol, 2.0 eq.) were dissolved in acetonitrile (20 mL), and the mixture was reacted at 90° C. for 26 h. After the reaction was completed as monitored by TLC, the reaction solution was concentrated under reduced pressure, and the crude product was dissolved in EA (20 mL). Water (20 mL) was added, and the pH was adjusted to 9 with sodium carbonate, followed by liquid separation. The aqueous phase was extracted with EA (20 mL×2), and the organic phases were combined, dried and concentrated. The crude product was purified by silica gel column chromatography (PE:EA=6:1) to give a mixture (0.5 g, yield: 39.4%) of 1-chloro-5-fluoro-4-(2-methoxy-4-methylphenyl)phthalazine and 4-chloro-5-fluoro-1-(2-methoxy-4-methylphenyl)phthalazine.

### Step 4: Synthesis of tert-butyl (R)-3-((5-fluoro-4-(2-methoxy-4-methylphenyl)phthalazin-1-yl)amino)piperidine-1-carboxylate and tert-butyl (R)-3-((8-fluoro-4-(2-methoxy-4-methylphenyl)phthalazin-1-yl)amino)piperidine-1-carboxylate

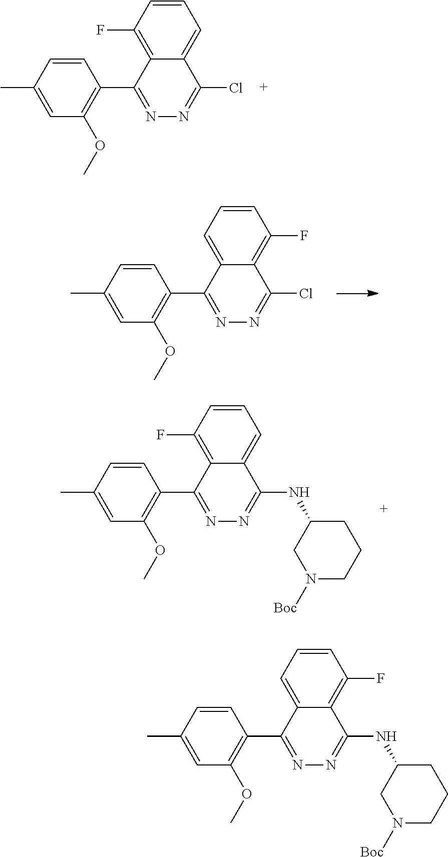

The mixture (0.5 g, 1.65 mmol, 1.0 eq.) of 1-chloro-5-fluoro-4-(2-methoxy-4-methylphenyl)phthalazine and 4-chloro-5-fluoro-1-(2-methoxy-4-methylphenyl)phthalazine, tert-butyl (R)-3-aminopiperidine-1-carboxylate (396 mg, 1.98 mmol, 1.2 eq.), Pd₂(dba)₃ (146 mg, 0.16 mmol, 0.1 eq.), BINAP (205 mg, 0.33 mmol, 0.2 eq.) and cesium carbonate (1.08 g, 3.30 mmol, 2.0 eq.) were dissolved in 1,4-dioxane (10 mL), and the mixture was reacted for 22 h at 80° C. in nitrogen atmosphere. After the reaction was completed as detected by LC-MS, the reaction solution was concentrated under reduced pressure, and the crude product was purified by silica gel column chromatography (MeOH:DCM=1:60) to give a mixture (400 mg, yield: 52.0%) of tert-butyl (R)-3-((5-fluoro-4-(2-methoxy-4-methylphenyl)phthalazin-1-yl)amino)piperidine-1-carboxylate and tert-butyl (R)-3-((8-fluoro-4-(2-methoxy-4-methylphenyl)phthalazin-1-yl)amino)piperidine-1-carboxylate.

### Step 5: Synthesis of (R)-2-(8-fluoro-4-(piperidin-3-ylamino)phthalazin-1-yl)-5-methylphenol and (R)-2-(5-fluoro-4-(piperidin-3-ylamino)phthalazin-1-yl)-5-methylphenol

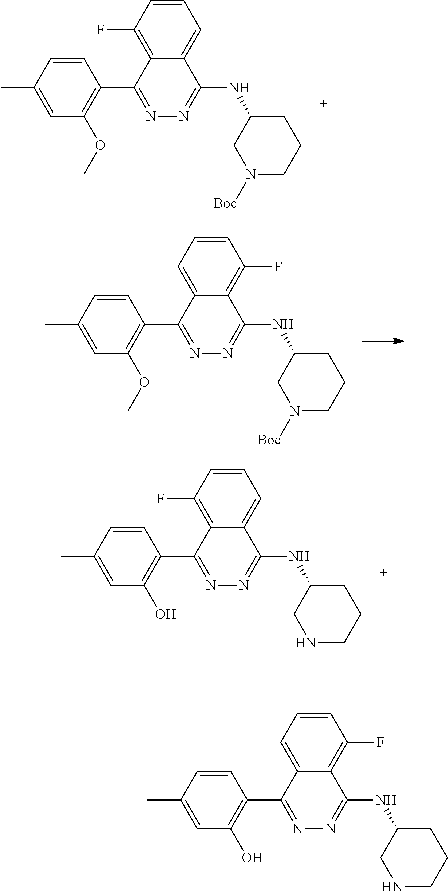

The mixture (0.4 g, 0.85 mmol, 1.0 eq.) of tert-butyl (R)-3-((5-fluoro-4-(2-methoxy-4-methylphenyl)phthalazin-1-yl)amino)piperidine-1-carboxylate and tert-butyl (R)-3-((8-fluoro-4-(2-methoxy-4-methylphenyl)phthalazin-1-yl)amino)piperidine-1-carboxylate was dissolved in DCM (20 mL), and the mixture was cooled to −20° C., followed by dropwise addition of a 1 mol/L solution of boron tribromide in dichloromethane (2.6 mL, 2.55 mmol, 3.0 eq.). After the completion of dropwise addition, the reaction solution was naturally warmed to room temperature and reacted for 1 h. After the reaction was completed as detected by LC-MS, an appropriate amount of methanol was added into the flask to quench the reaction. The reaction solution was concentrated under reduced pressure, and the crude product was dissolved completely in DCM (10 mL), followed by addition of water (20 mL) and extraction with DCM (10 mL×2). The pH of the aqueous phase was adjusted to 9 with NaHCO₃, followed by extraction with MeOH:DCM (1:10) (15 mL×2). The organic phase was dried and concentrated to give a mixture (80 mg, yield: 26.7%) of (R)-2-(8-fluoro-4-(piperidin-3-ylamino)phthalazin-1-yl)-5-methylphenol and (R)-2-(5-fluoro-4-(piperidin-3-ylamino)phthalazin-1-yl)-5-methylphenol.

### Step 6: Synthesis of (R)-2-(8-fluoro-4-((1-methylpiperidin-3-yl)amino)phthalazin-1-yl)-5-methylphenol (Compound 6) and (R)-2-(5-fluoro-4-((1-methylpiperidin-3-yl)amino)phthalazin-1-yl)-5-methylphenol (Compound 9)

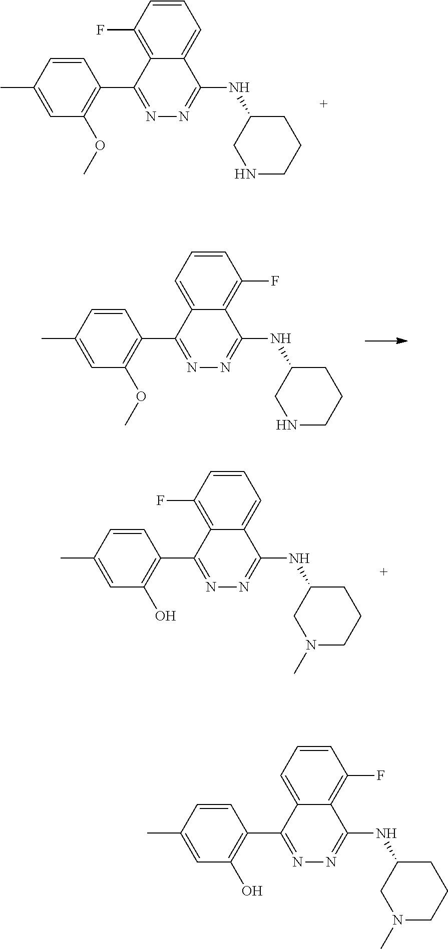 [SMILES: COc1cc(C)ccc1-c1nnc(N[C@@H]2CCCNC2)c2cccc(F)c12 ; COc1cc(C)ccc1-c1nnc(N[C@@H]2CCCNC2)c2c(F)cccc12 ; Cc1ccc(-c2nnc(N[C@@H]3CCCN(C)C3)c3cccc(F)c23)c(O)c1 ; Cc1ccc(-c2nnc(N[C@@H]3CCCN(C)C3)c3c(F)cccc23)c(O)c1]

The mixture (80 mg, 0.22 mmol, 1.0 eq.) of (R)-2-(8-fluoro-4-(piperidin-3-ylamino)phthalazin-1-yl)-5-methylphenol and (R)-2-(5-fluoro-4-(piperidin-3-ylamino)phthalazin-1-yl)-5-methylphenol was dissolved in methanol (2 mL), followed by addition of an aqueous formaldehyde solution (37%) (18 mg, 0.22 mmol, 1.0 eq.). The resulting mixture was stirred at room temperature for 5 min, followed by addition of sodium cyanoborohydride (16 mg, 0.22 mmol, 1.0 eq.). The mixture was reacted at room temperature for 5 min. After the generation of the target product as monitored by LC-MS, the reaction solution was concentrated under reduced pressure, and the crude product was purified by preparative thin-layer chromatography (DCM:MeOH=7:1) to give the product (compound 6) (R)-2-(8-fluoro-4-((1-methylpiperidin-3-yl)amino)phthalazin-1-yl)-5-methylphenol with a smaller Rf value (10 mg, yield: 12.5%).

¹HNMR (400 MHz, DMSO-d6) δ (ppm): 9.26 (s, 1H), 8.24-8.22 (d, 1H), 7.90-7.85 (m, 1H), 7.58-7.54 (m, 1H), 7.27 (s, 1H), 7.15-7.13 (d, 1H), 6.70-6.69 (d, 1H), 4.52 (s, 1H), 2.97 (s, 1H), 2.51 (s, 3H), 2.50 (s, 2H), 2.30 (s, 3H), 2.02-2.00 (d, 3H), 1.86 (s, 1H), 1.71-1.69 (d, 1H).

Molecular formula: C₂₁H₂₃FN₄O Molecular weight: 366.44 LC-MS (Pos, m/z)=367.20[M+H]⁺.

Simultaneously, the product (compound 9) (R)-2-(5-fluoro-4-((1-methylpiperidin-3-yl)amino)phthalazin-1-yl)-5-methylphenol with a larger Rf value (15 mg, yield: 18.8%) is also given.

¹HNMR (400 MHz, DMSO-d6) δ (ppm): 9.51 (s, 1H), 7.82-7.76 (m, 1H), 7.70-7.65 (m, 1H), 7.33-7.31 (d, 1H), 7.15-7.13 (d, 1H), 6.81 (s, 1H), 6.78-6.76 (d, 1H), 6.69-6.65 (m, 1H), 4.56 (s, 1H), 2.95 (s, 1H), 2.52 (s, 1H), 2.50 (s, 3H), 2.42-2.40 (d, 2H), 2.33 (s, 3H), 1.80 (s, 3H), 1.66 (s, 1H).

The two-dimensional NOE spectrum showed coupling signals between 7.33-7.31 (d, 1H) and 7.15-7.13 (d, 1H) and coupling signals between 7.33-7.31 (d, 1H) and 9.51 (s, 1H).

Molecular formula: C₂₁H₂₃FN₄O Molecular weight: 366.44 LC-MS (Pos, m/z)=367.22[M+H]⁺.

### Example 14: Synthesis of (R)-2-(6-methoxy-4-((1-methylpiperidin-3-yl)amino)phthalazin-1-yl)-5-methylphenol (Compound 16)

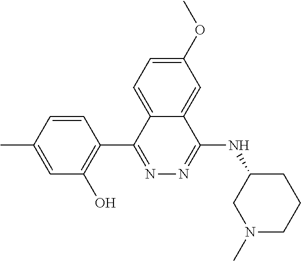 [SMILES: COc1ccc2c(-c3ccc(C)cc3O)nnc(N[C@@H]3CCCN(C)C3)c2c1]

### Step 1: Synthesis of (R)-2-(6-methoxy-4-((1-methylpiperidin-3-yl)amino)phthalazin-1-yl)-5-methylphenol

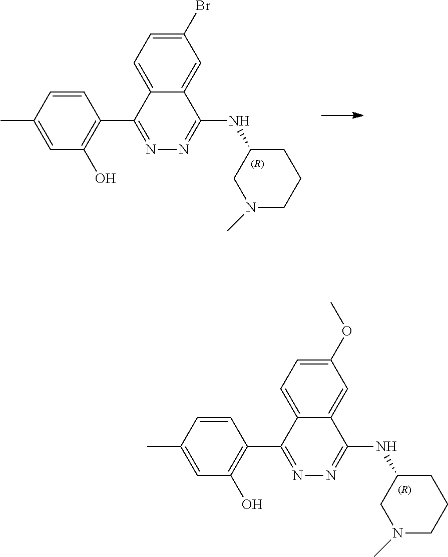

(R)-2-(6-bromo-4-((1-methylpiperidin-3-yl)amino)phthalazin-1-yl)-5-methylphenol (100 mg, 0.23 mmol, 1.0 eq.), 2-di-tert-butylphosphino-2′,4′,6′-triisopropylbiphenyl (21 mg, 0.05 mmol, 0.2 eq.), cesium carbonate (150 mg, 0.46 mmol, 2.0 eq.) and allylpalladium chloride dimer (18 mg, 0.05 mmol, 0.2 eq.) were added to a mixed solution of MeOH (1 mL) and dimethylbenzene (2 mL), and the mixture was reacted at 80° C. for 1 h in nitrogen atmosphere. After the reaction was completed as monitored by LC-MS, the reaction solution was concentrated under reduced pressure, and the crude product was purified by preparative thin-layer chromatography (MeOH:DCM=1:7) to give the product (15 mg, yield: 17.2%).

¹HNMR (300 MHz, DMSO-d6) δ (ppm): 7.74 (s, 1H), 7.50-7.47 (d, 1H), 7.40-7.37 (d, 1H), 7.17-7.15 (d, 1H), 6.80-6.75 (d, 2H), 4.48 (s, 1H), 3.96 (s, 3H), 3.42 (s, 3H), 2.88 (s, 1H), 2.38 (s, 3H), 2.33 (s, 4H), 2.19-2.02 (d, 2H), 1.70-1.54 (d, 2H).

Molecular formula: C₂₂H₂₆N₄O₂ Molecular weight: 378.48 LC-MS (Pos, m/z): 379.17[M+H]⁺.

### Example 15: Synthesis of (R)-1-(2-hydroxy-4-methylphenyl)-4-((1-methylpiperidin-3-yl)amino)phthalazine-6-carbonitrile (Compound 20)

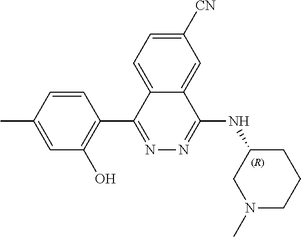

### Step 1: Synthesis of (R)-1-(2-hydroxy-4-methylphenyl)-4-((1-methylpiperidin-3-yl)amino)phthalazine-6-carbonitrile

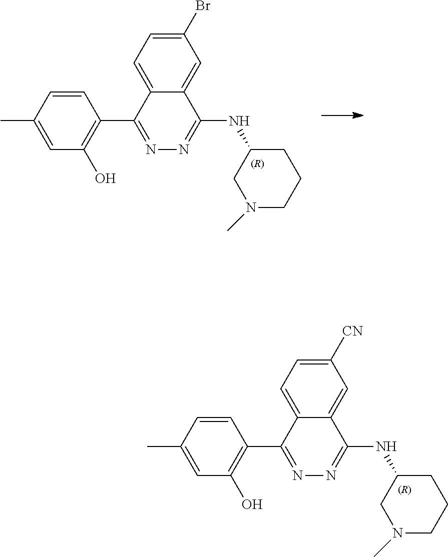

(R)-2-(6-bromo-4-((1-methylpiperidin-3-yl)amino)phthalazin-1-yl)-5-methylphenol (120 mg, 0.28 mmol, 1.0 eq.), zinc cyanide (33 mg, 0.28 mmol, 1.0 eq.) and tetrakis(triphenylphosphine)palladium (65 mg, 0.026 mmol, 0.2 eq.) were dissolved in DMAC (2 mL), and the mixture was reacted at 120° C. for 16 h in nitrogen atmosphere. After the reaction was completed as monitored by LC-MS, the reaction solution was cooled to room temperature and then poured into water (10 mL), followed by extraction with EA (10 mL×3). The organic phases were combined, washed with water (15 mL×3) and concentrated under reduced pressure, and the crude product was purified by preparative thin-layer chromatography (MeOH:DCM=1:8) to give the product (50 mg, yield: 48.1%).

¹HNMR (300 MHz, DMSO-d6) δ (ppm): 9.61 (s, 1H), 9.08 (s, 1H), 8.13-8.11 (d, 1H), 7.61-7.58 (d, 1H), 7.50-7.52 (d, 1H), 7.19-7.17 (d, 1H), 6.83-6.78 (t, 2H), 4.53 (s, 1H), 3.41 (s, 4H), 2.96-2.92 (d, 1H), 2.45 (s, 2H), 2.34 (s, 3H), 2.02-1.88 (d, 2H), 1.73-1.59 (m, 2H).

Molecular formula: C₂₂H₂₃N₅O Molecular weight: 373.46 LC-MS (Pos, m/z): 374.17[M+H]⁺.

### Example 16: Synthesis of (R)-2-(6-cyclopropyl-4-((1-methylpiperidin-3-yl)amino)phthalazin-1-yl)-5-methylphenol (Compound 32)

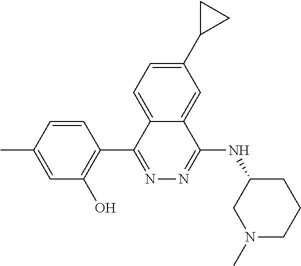 [SMILES: Cc1ccc(-c2nnc(N[C@@H]3CCCN(C)C3)c3cc(C4CC4)ccc23)c(O)c1]

### Step 1: Synthesis of tert-butyl (R)-3-((7-cyclopropyl-4-(2-methoxy-4-methylphenyl)phthalazin-1-yl)amino)piperidine-1-carboxylate

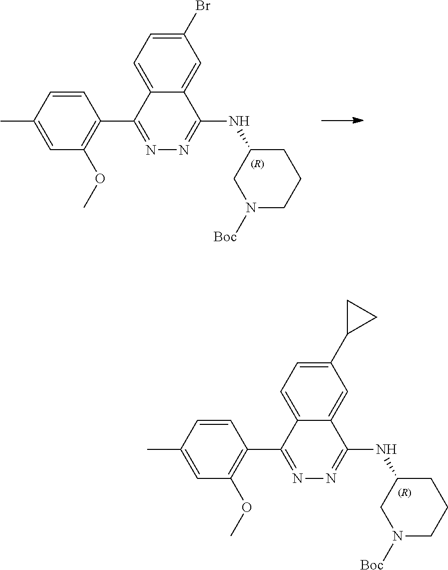

Tert-butyl (R)-3-((7-bromo-4-(2-methoxy-4-methylphenyl)phthalazin-1-yl)amino)piperidine-1-carboxylate (0.5 g, 0.94 mmol, 1.0 eq.), cyclopropylboronic acid (242 mg, 2.82 mmol, 3.0 eq.), tricyclohexylphosphorus (131 mg, 0.47 mmol, 0.5 eq.), palladium acetate (43 mg, 0.19 mmol, 0.2 eq.) and potassium phosphate (399 mg, 1.88 mmol, 2.0 eq.) were added to a mixed solution of toluene (10 mL) and water (2 mL), and the mixture was reacted at 100° C. for 2 h in nitrogen atmosphere. After the reaction was completed as detected by TLC, the reaction solution was poured into water (10 mL), and EA (10 mL×3) was added for extraction. The organic phase was dried and concentrated to give the product (400 mg, yield: 87.1%).

### Step 2: Synthesis of (R)-7-cyclopropyl-4-(2-methoxy-4-methylphenyl)-N-(piperidin-3-yl)phthalazin-1-amine

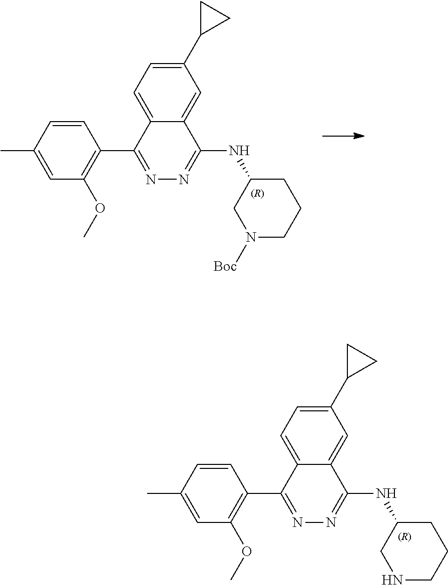

Tert-butyl (R)-3-((7-cyclopropyl-4-(2-methoxy-4-methylphenyl)phthalazin-1-yl)amino)piperidine-1-carboxylate (400 mg, 0.81 mmol, 1.0 eq.) was dissolved in DCM (10 mL), followed by dropwise addition of a 1 mol/L solution of boron tribromide in dichloromethane (2.4 mL, 2.43 mmol, 3.0 eq.) at room temperature. The mixture was reacted at room temperature for 20 min. After the reaction was completed as detected by LC-MS, an appropriate amount of methanol was added to the system to quench the reaction. The reaction solution was concentrated under reduced pressure, and the crude product was dissolved in water (10 mL). The aqueous phase was extracted with DCM (10 mL×2), and the aqueous phase was retained. The pH was adjusted to 9 with NaHCO₃, followed by extraction with DCM (10 mL×4). The organic phases were combined, dried and concentrated to give the product (150 mg, yield: 47.7%).

### Step 3: Synthesis of (R)-2-(6-cyclopropyl-4-((1-methylpiperidin-3-yl)amino)phthalazin-1-yl)-5-methylphenol

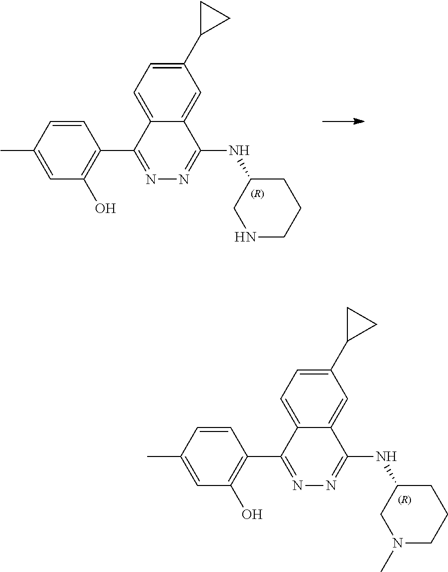

(R)-7-cyclopropyl-4-(2-methoxy-4-methylphenyl)-N-(piperidin-3-yl) phthalazin-1-amine (150 mg, 0.40 mmol, 1.0 eq.) was dissolved in methanol (3 mL), followed by addition of an aqueous formaldehyde solution (37%) (32 mg, 0.40 mmol, 1.0 eq.). The resulting mixture was stirred at room temperature for 5 min, followed by addition of sodium cyanoborohydride (25 mg, 0.40 mmol, 1.0 eq.). The mixture was reacted at room temperature for 5 min. After the reaction was completed as monitored by LC-MS, the reaction solution was concentrated under reduced pressure, and water (5 mL) and DCM (10 mL) were added to the system, followed by liquid separation. The aqueous phase was extracted with DCM (10 mL×2), and the organic phases were combined, dried and concentrated. The crude product was purified by preparative thin-layer chromatography (DCM:MeOH=7:1) to give the product (45 mg, yield: 29.0%).

¹HNMR (300 MHz, DMSO-d6) δ (ppm): 8.03 (s, 1H), 7.54-7.51 (d, 1H), 7.43-7.41 (d, 1H), 7.16-7.14 (d, 1H), 6.80-6.75 (t, 2H), 4.54 (s, 1H), 3.43 (s, 5H), 2.99 (s, 1H), 2.51 (s, 4H), 2.33 (s, 3H), 2.16-2.10 (m, 1H), 2.02-1.88 (t, 2H), 1.74-1.62 (m, 2H), 1.13-1.10 (t, 2H), 0.92-0.91 (d, 2H).

Molecular formula: C₂₄H₂₈N₄O Molecular weight: 388.52 LC-MS (Pos, m/z): 389.23[M+H]⁺.

### Example 17: Synthesis of (R)-5-methyl-2-(4-((1-methylpiperidin-3-yl)amino)thieno[2,3-d]pyridazin-7-yl)phenol (Compound 48)

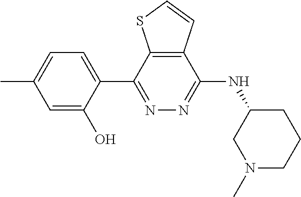 [SMILES: Cc1ccc(-c2nnc(N[C@@H]3CCCN(C)C3)c3ccsc23)c(O)c1]

### Step 1: Synthesis of tert-butyl (R)-3-((7-chlorothieno[2,3-d]pyridazin-4-yl)amino)piperidine-1-carboxylate

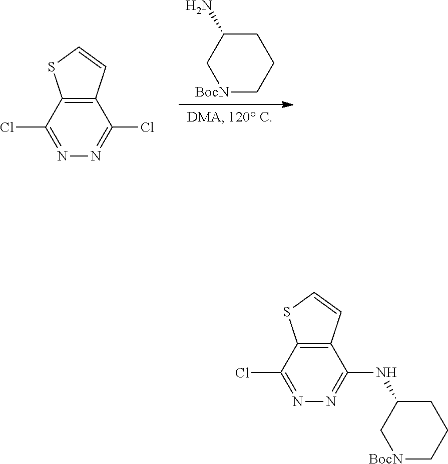

4,7-Dichlorothieno[2,3-d]pyridazine (1.0 g, 4.88 mmol, 1.0 eq.) and tert-butyl (R)-3-aminopiperidine-1-carboxylate (1.17 g, 5.86 mmol, 1.2 eq.) were dissolved in DMA (10 mL), and the mixture was heated to 120° C. and reacted for 20 h in nitrogen atmosphere. Water (50 mL) was added to quench the reaction, and EA (50 mL) was added for extraction. The organic phase was washed with water (30 mL×3), dried over anhydrous sodium sulfate and filtered, and the filtrate was concentrated under reduced pressure. The crude product was separated by silica gel column chromatography (DCM:MeOH=100:1 to 50:1) to give the product (1.40 g, yield: 77.8%).

### Step 2: Synthesis of tert-butyl (R)-3-((7-(2-methoxy-4-methylphenyl)thieno[2,3-d]pyridazin-4-yl)amino)piperidine-1-carboxylate

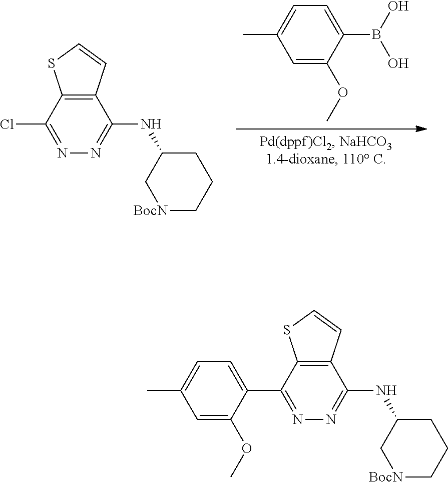

(2-Methoxy-4-methylphenyl)boronic acid (0.630 g, 3.80 mmol, 1.0 eq.), tert-butyl (R)-3-((7-chlorothieno[2,3-d]pyridazin-4-yl)amino)piperidine-1-carboxylate (1.40 g, 3.80 mmol, 1.0 eq.), Pd(dppf)Cl₂ (0.139 g, 0.190 mmol, 0.05 eq.) and NaHCO₃ (0.638 g, 7.60 mmol, 2.0 eq.) were added successively to 1,4-dioxane (20 mL), followed by addition of H₂O (10 mL). The mixture was heated to 110° C. and reacted for 6 h in nitrogen atmosphere. Water (60 mL) was added, and EA (50 mL) was added for extraction. The organic phase was dried over anhydrous sodium sulfate and filtered, and the filtrate was concentrated under reduced pressure. The crude product was separated by silica gel column chromatography (DCM:MeOH=100:1 to 20:1) to give the product (920 mg, yield: 53.3%).

### Step 3: Synthesis of (R)-5-methyl-2-(4-(piperidin-3-ylamino)thieno[2,3-d]pyridazin-7-yl)phenol

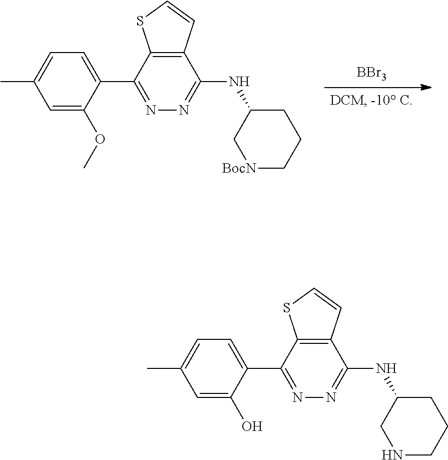 [SMILES: COc1cc(C)ccc1-c1nnc(N[C@@H]2CCCN(C(=O)OC(C)(C)C)C2)c2ccsc12 ; "BBr3" ; "DCM, -10° C." ; Cc1ccc(-c2nnc(N[C@@H]3CCCNC3)c3ccsc23)c(O)c1]

Tert-butyl (R)-3-((7-(2-methoxy-4-methylphenyl)thieno[2,3-d]pyridazin-4-yl)amino)piperidine-1-carboxylate (900 mg, 1.98 mmol, 1.0 eq.) was dissolved in DCM (18 mL), and the mixture was cooled to −10° C., followed by dropwise addition of BBr₃ (1.98 g, 7.92 mmol, 4.0 eq.). The mixture was reacted for 4 h. MeOH (5 mL) was added to quench the reaction, a saturated aqueous NaHCO₃ solution was added to adjust the pH to 8, and DCM (30 mL×2) was added for extraction. The organic phase was dried over anhydrous sodium sulfate and filtered, and the filtrate was concentrated under reduced pressure. The crude product was purified by silica gel column chromatography (DCM:MeOH=50:1 to 10:1) to give the product (490 mg, yield: 72.7%).

### Step 4: Synthesis of (R)-5-methyl-2-(4-((1-methylpiperidin-3-yl)amino)thieno[2,3-d]pyridazin-7-yl)phenol

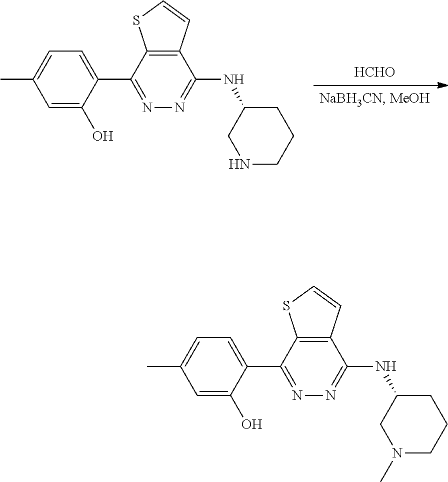

(R)-5-methyl-2-(4-(piperidin-3-ylamino)thieno[2,3-d]pyridazin-7-yl)phenol (490 mg, 1.44 mmol, 1.0 eq.) was dissolved in MeOH (4 mL), followed by addition of an aqueous formaldehyde solution (37%) (351 mg, 4.32 mmol, 3.0 eq.). The mixture was stirred at room temperature for 30 min, followed by addition of NaBH₃CN (271 mg, 4.32 mmol, 3.0 eq.). The resulting mixture was reacted for 1 h. A saturated aqueous NaHCO₃ solution was added to quench the reaction, and DCM (30 mL×2) was added for extraction. The organic phase was dried over anhydrous sodium sulfate and filtered, and the filtrate was concentrated under reduced pressure. The crude product was separated by preparative thin-layer chromatography (DCM:MeOH=15:1) to give the product (120 mg, yield: 23.5%).

¹HNMR (400 MHz, DMSO-d6) δ (ppm): 12.26 (s, 1H), 8.25 (d, J=5.2 Hz, 1H), 8.07 (d, J=5.2 Hz, 1H), 7.78 (d, J=8.4 Hz, 1H), 7.32 (d, J=6.8 Hz, 1H), 6.84 (d, J=6.4 Hz, 2H), 4.40 (s, 1H), 3.16 (d, J=9.6 Hz, 1H), 2.81 (d, J=10.3 Hz, 1H), 2.32 (s, 6H), 2.09-1.99 (m, 3H), 1.82-1.79 (m, 1H), 1.69-1.60 (m, 1H), 1.50-1.45 (m, 1H).

Molecular formula: C₁₉H₂₂N₄OS Molecular weight: 354.47 LC-MS (Pos, m/z)=355.13[M+H]⁺.

### Example 18: Synthesis of (R)-5-methyl-2-(4-((1-methylpiperidin-3-yl)amino)pyrido[3,4-d]pyridazin-1-yl)phenol (Compound 50)

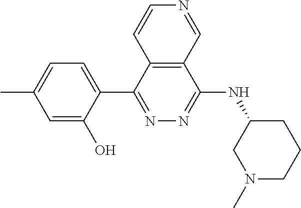 [SMILES: Cc1ccc(-c2nnc(N[C@@H]3CCCN(C)C3)c3cnccc23)c(O)c1]

### Step 1: Synthesis of Intermediate 4-(2-methoxy-4-methylbenzoyl)nicotinic acid

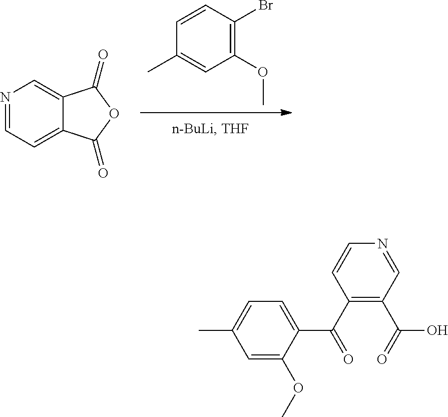

1-Bromo-2-methoxy-4-methylbenzene (14.16 g, 70.41 mmol, 1.05 eq.) was dissolved in anhydrous THE (100 mL), followed by dropwise addition of n-BuLi (32.2 mL, 80.47 mmol, 1.2 eq.) at −78° C. After the completion of dropwise addition, the mixture was reacted for 30 min. At −78° C., the reaction solution was added dropwise to a solution of 3,4-pyridine dicarboxylic anhydride (10 g, 67.06 mmol, 1.0 eq.) in THE (100 mL). After the completion of dropwise addition, the mixture was reacted at room temperature for 1 h. After the reaction was completed as detected by TLC, the reaction solution was poured into a mixed solution of ice water (50 mL) and 2 mol/L hydrochloric acid (50 mL), followed by extraction with dichloromethane (200 mL×3). The organic phases were combined, dried and concentrated to give the crude product (20 g). 5 g of the crude product was subjected to silica gel column chromatography (petroleum ether:ethyl acetate=1:3 (containing 0.5% AcOH), dichloromethane:methanol=10:1 (containing 0.5% AcOH)) to give the product (2.5 g, yield: 55.6%).

### Step 2: Synthesis of Intermediate methyl 4-(2-methoxy-4-methylbenzoyl)nicotinate

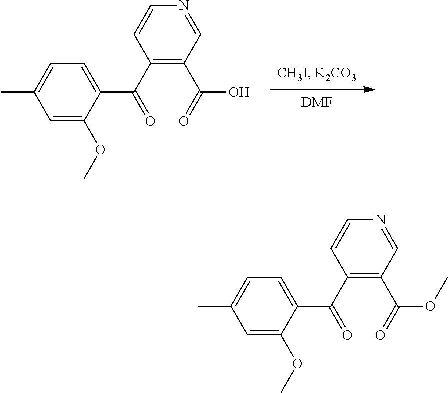

4-(2-Methoxy-4-methylbenzoyl)nicotinic acid (2.5 g, 9.22 mmol, 1.0 eq.) was dissolved in DMF (30 mL), followed by addition of potassium carbonate (1.91 g, 13.83 mmol, 1.5 eq.) and iodomethane (1.31 g, 9.22 mmol, 1.0 eq.). The mixture was reacted at room temperature for 2 h. After the reaction was completed as detected by TLC, the reaction solution was poured into water (20 mL), followed by extraction with EA (50 mL×3). The organic phases were combined, dried over anhydrous sodium sulfate and concentrated to give the product (1.1 g, yield: 41.8%).

### Step 3: Synthesis of Intermediate 1-(2-methoxy-4-methylphenyl)pyrido[3,4-d]pyridazin-4-ol

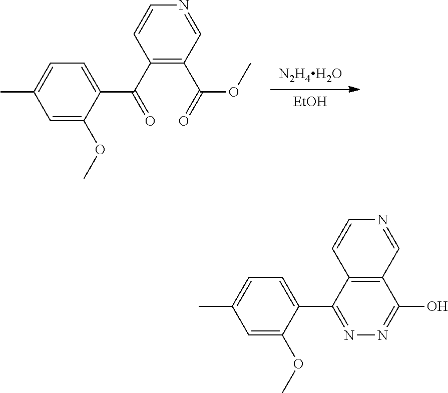

Methyl 4-(2-methoxy-4-methylbenzoyl)nicotinate (1.1 g, 3.86 mmol, 1.0 eq.) was dissolved in ethanol (10 mL), followed by addition of 85% hydrazine hydrate (341 mg, 5.79 mmol, 1.5 eq.). The mixture was heated to 85° C. and reacted for 10 min. After the reaction was completed as detected by LC-MS, the reaction solution was cooled to room temperature, and solids precipitated. The mixture was filtered to give the product (640 mg, yield: 62.1%).

### Step 4: Synthesis of Intermediate 4-chloro-1-(2-methoxy-4-methylphenyl)pyrido[3,4-d]pyridazine

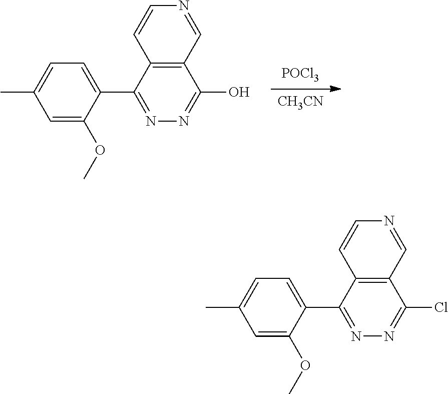

Acetonitrile (8 mL) was added to 1-(2-methoxy-4-methylphenyl)pyrido[3,4-d]pyridazin-4-ol (640 mg, 2.39 mmol, 1.0 eq.), followed by addition of phosphorus oxychloride (733 mg, 4.78 mmol, 2.0 eq.). The mixture was reacted at 90° C. for 16 h. After the reaction was completed as detected by TLC, the reaction solution was concentrated, and the residue was poured into ice water (10 mL), followed by extraction with dichloromethane (20 mL). The organic phase was dried and concentrated, and the crude product was subjected to silica gel column chromatography (petroleum ether:ethyl acetate=3:1) to give the product (536 mg, yield: 78.4%).

### Step 5: Synthesis of Intermediate tert-butyl (R)-3-((1-(2-methoxy-4-methylphenyl)pyrido[3,4-d]pyridazin-4-yl)amino)piperidine-1-carboxylate

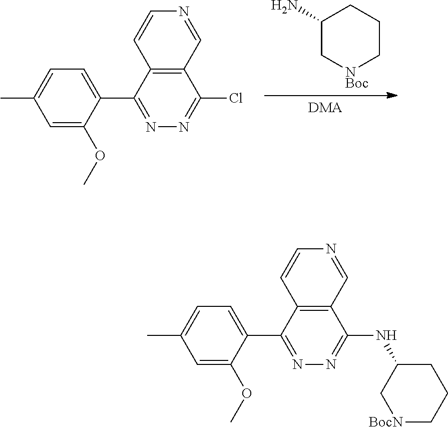

4-Chloro-1-(2-methoxy-4-methylphenyl)pyrido[3,4-d]pyridazine (536 mg, 1.876 mmol, 1.0 eq.) was dissolved in DMA (5 mL), followed by addition of (R)-1-tert-butoxycarbonyl-3-aminopiperidine (752 mg, 3.752 mmol, 2.0 eq.). The mixture was refluxed at 120° C. for 16 h. After the reaction was completed as detected by TLC, the reaction solution was added dropwise into water (5 mL), ethyl acetate (10 mL) was added for extraction, and DCM (10 mL×3) was added for extraction. The organic phases were combined, dried and concentrated to give the product (647 mg, yield: 76.7%).

### Step 6: Synthesis of Intermediate (R)-5-methyl-2-(4-((piperidin-3-yl)amino)pyrido[3,4-d]pyridazin-1-yl)phenol

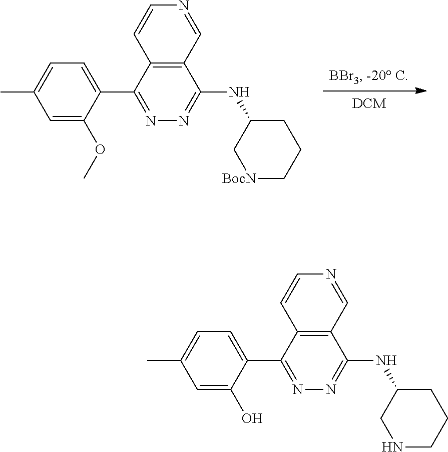

Tert-butyl (R)-3-((1-(2-methoxy-4-methylphenyl)pyrido[3,4-d]pyridazin-4-yl)amino)piperidine-1-carboxylate (647 mg, 1.44 mmol, 1.0 eq.) was dissolved in DCM (5 mL), followed by dropwise addition of boron tribromide (1.08 g, 4.32 mmol, 3.0 eq.) at −20° C. The mixture was reacted at room temperature for 1 h. After there remained some of the materials as detected by LC-MS, the reaction solution was supplemented with boron tribromide (1.08 g, 4.32 mmol, 3.0 eq.). The resulting mixture was reacted at room temperature for 1 h. After the reaction was completed as detected by LC-MS, methanol (3 mL) and water (10 mL) were added dropwise and slowly to the reaction solution under an ice bath, followed by back-extraction with DCM (10 mL). The pH of the aqueous phase was adjusted to be neutral with a saturated aqueous sodium bicarbonate solution, followed by back-extraction with DCM (10 mL). The aqueous phase was concentrated, dissolved in a mixed solvent of DCM/MeOH, dried over anhydrous magnesium sulfate and concentrated, and the crude product was directly used in the next step.

### Step 7: Synthesis of compound (R)-5-methyl-2-(4-((1-methylpiperidin-3-yl)amino)pyrido[3,4-d]pyridazin-1-yl)phenol

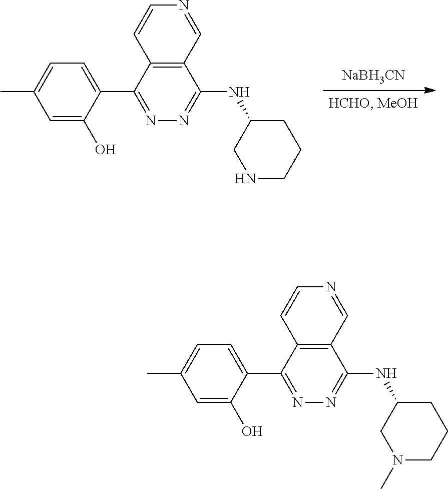

(R)-5-methyl-2-(4-((piperidin-3-yl)amino)pyrido[3,4-d]pyridazin-1-yl)phenol (1.44 mmol, 1.0 eq.) was dissolved in methanol (5 mL), followed by addition of an aqueous acetaldehyde solution (38.5%) (112 mg, 1.44 mmol, 1.0 eq.). The mixture was stirred at room temperature for 5 min, followed by addition of sodium cyanoborohydride (100 mg, 1.584 mmol, 1.1 eq.). The resulting mixture was reacted at room temperature for 5 min. After the reaction was completed as detected by TLC, the reaction solution was concentrated, and the crude product was purified by preparative thin-layer chromatography (dichloromethane:methanol=7:1) to give the product (12 mg, two-step yield: 2.3%).

¹H-NMR (300 MHz, DMSO-d6) δ (ppm): 9.86 (brs, 1H), 9.67 (s, 1H), 8.86-8.88 (d, 1H), 7.84 (s, 1H), 7.33-7.35 (d, 1H), 7.19-7.21 (d, 1H), 6.79-6.84 (m, 2H), 4.66 (s, 1H), 3.13 (s, 3H), 2.64-2.74 (m, 4H), 2.34 (s, 3H), 1.77-2.02 (m, 4H).

Molecular formula: C₂₀H₂₃N₅O Molecular weight: 349.44 LC-MS=350.23[M+H]⁺

### Example 19: Synthesis of Compound (R)-3-hydroxy-4-(8-((1-methylpiperidin-3-yl)amino)pyrido[2,3-d]pyridazin-5-yl)benzonitrile (Compound 59)

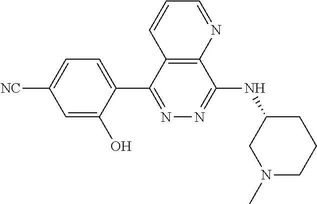 [SMILES: CN1CCC[C@@H](Nc2nnc(-c3ccc(C#N)cc3O)c3cccnc23)C1]

### Step 1: Synthesis of Intermediate 3-(4-bromo-2-methoxybenzoyl)nicotinic acid

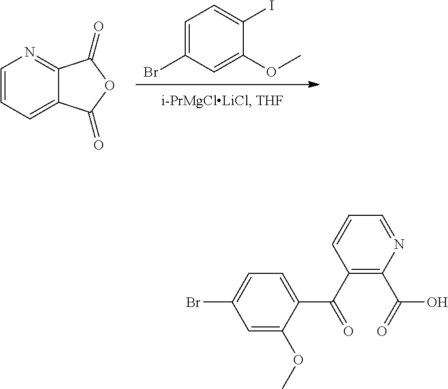

4-Bromo-1-iodo-2-methoxybenzene (10 g, 31.96 mmol, 1.0 eq.) was dissolved in anhydrous THE (100 mL), followed by dropwise addition of a solution of i-PrMgCl LiCl in THE (1.3 mol/L, 26 mL, 33.6 mmol, 1.05 eq.) at −20° C. After the completion of dropwise addition, the mixture was reacted for 30 min. At −20° C., the above reaction solution was added dropwise to a solution of 2,3-pyridine dicarboxylic anhydride (4.77 g, 31.96 mmol, 1.0 eq.) in THE (100 mL). After the completion of dropwise addition, the mixture was reacted at room temperature for 16 h. After the reaction was completed as detected by TLC, the reaction solution was poured into a saturated aqueous ammonium chloride solution (200 mL), followed by concentration and extraction with dichloromethane (200 mL×5). The organic phases were combined, dried over anhydrous magnesium sulfate and concentrated, and the crude product was purified by silica gel column chromatography (petroleum ether:ethyl acetate=2:1, v/v, with 5% acetic acid added) to give the product (3.9 g, yield: 36.3%).

### Step 2: Synthesis of Intermediate 5-(4-bromo-2-methoxyphenyl)pyrido[2,3-d]pyridazin-8-ol

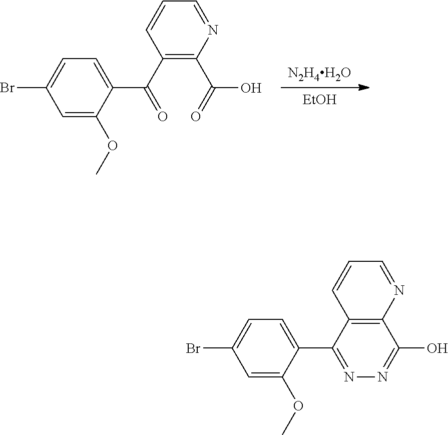 [SMILES: "EtOH" ; COc1cc(Br)ccc1C(=O)c1cccnc1C(=O)O ; "N2H4·H2O" ; COc1cc(Br)ccc1-c1nnc(O)c2ncccc12]

3-(4-Bromo-2-methoxybenzoyl)nicotinic acid (3.9 g, 11.6 mmol, 1.0 eq.) was added to EtOH (40 mL), followed by addition of 85% hydrazine hydrate (1.37 g, 23.2 mmol, 2.0 eq.). The mixture was heated to 90° C. and reacted for 8 h. After the reaction was completed as detected by TLC, the reaction solution was concentrated and filtered, and the filter cake was washed with ethanol to give the product (2.02 g, yield: 52.4%).

### Step 3: Synthesis of Intermediate 5-(4-bromo-2-methoxyphenyl)-8-chloropyrido[2,3-d]pyridazine

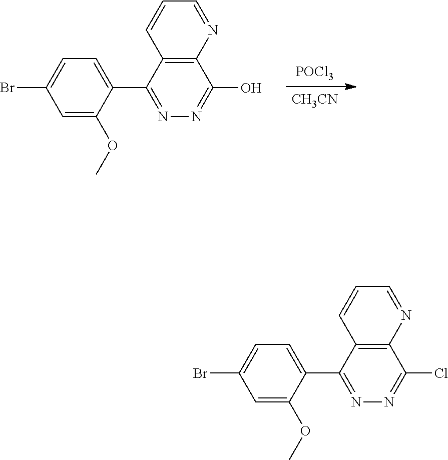

5-(4-Bromo-2-methoxyphenyl)pyrido[2,3-d]pyridazin-8-ol (2.02 g, 6.08 mmol, 1.0 eq.) was added to acetonitrile (20 mL), followed by addition of phosphorus oxychloride (1.86 g, 12.16 mmol, 2.0 eq.). The mixture was reacted at 100° C. for 16 h. After the reaction was completed as detected by TLC, the reaction solution was concentrated, and the concentrated solution was poured into ice water (10 mL). The pH was adjusted to alkalinity with sodium bicarbonate, followed by extraction with dichloromethane (30 mL×3). The organic phases were combined, dried and concentrated, and the crude product was purified by silica gel column chromatography (petroleum ether:ethyl acetate=5:1, v/v) to give the product (2.0 g, yield: 93.8%).

### Step 4: Synthesis of Intermediate 5-bromo-2-(8-chloropyrido[2,3-d]pyridazin-5-yl)phenol

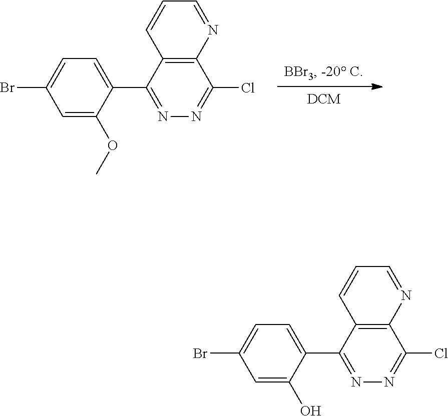

5-(4-Bromo-2-methoxyphenyl)-8-chloropyrido[2,3-d]pyridazine (2 g, 5.7 mmol, 1.0 eq.) was dissolved in dichloromethane (20 mL), followed by dropwise and slow addition of a solution of boron tribromide in n-heptane (1 mol/L, 8.6 mL, 8.6 mmol, 1.5 eq.) at −20° C. After the completion of dropwise addition, the mixture was reacted at room temperature for 10 min. After the reaction was completed as detected by TLC, a saturated aqueous sodium bicarbonate solution (10 mL) was added dropwise and slowly to the reaction solution under an ice bath, followed by extraction with DCM (20 mL×3). The organic phases were combined, dried and concentrated, and the crude product was purified by silica gel column chromatography (petroleum ether:ethyl acetate=2:1, v/v) to give the product (900 mg, yield: 47.3%).

### Step 5: Synthesis of Intermediate tert-butyl (R)-3-((5-(4-bromo-2-methoxyphenyl)pyrido[2,3-d]pyridazin-8-yl)amino)piperidine-1-carboxylate

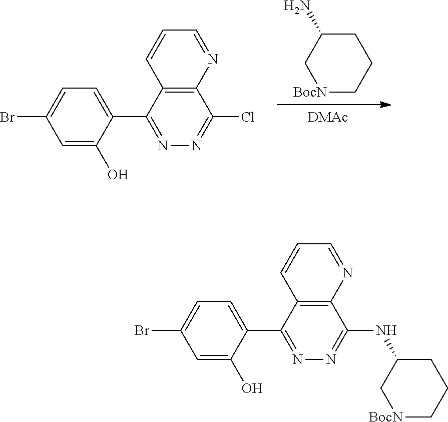

5-Bromo-2-(8-chloropyrido[2,3-d]pyridazin-5-yl)phenol (900 mg, 2.7 mmol, 1.0 eq.) was dissolved in DMAc (10 mL), followed by addition of (R)-1-tert-butoxycarbonyl-3-aminopiperidine (1.08 g, 5.4 mmol, 2.0 eq.). The mixture was refluxed at 120° C. for 1 h. After the reaction was completed as detected by TLC, the reaction solution was added dropwise into water (5 mL), followed by extraction with ethyl acetate (10 mL×2). The organic phases were combined, dried and concentrated, and the crude product was purified by silica gel column chromatography (dichloromethane:methanol=200:1) to give the product (1.05 g, yield: 78.3%). (1900326-179)

### Step 6: Synthesis of Intermediate tert-butyl (R)-3-((5-(4-cyano-2-hydroxyphenyl)pyrido[2,3-d]pyridazin-8-yl)amino)piperidine-1-carboxylate

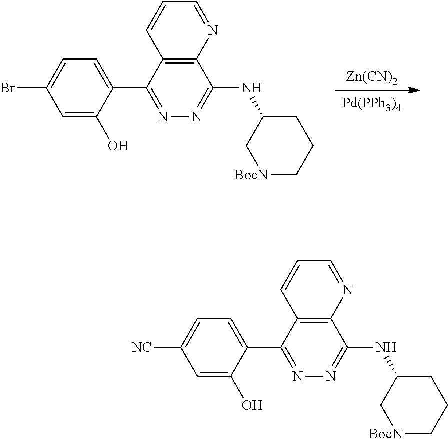 [SMILES: CC(C)(C)OC(=O)N1CCC[C@@H](Nc2nnc(-c3ccc(Br)cc3O)c3cccnc23)C1 ; "Zn(CN)2" ; CC(C)(C)OC(=O)N1CCC[C@@H](Nc2nnc(-c3ccc(C#N)cc3O)c3cccnc23)C1 ; "Pd(PPh3)4"]

Tert-butyl (R)-3-((5-(4-bromo-2-methoxyphenyl)pyrido[2,3-d]pyridazin-8-yl)amino)piperidine-1-carboxylate (500 mg, 1 mmol, 1.0 eq.) was dissolved in DMAc (5 mL), followed by addition of zinc cyanide (94 mg, 0.8 mmol, 0.8 eq.) and tetrakis(triphenylphosphine)palladium (112 mg, 0.1 mmol, 0.1 eq.) in nitrogen atmosphere. The mixture was reacted at 120° C. for 45 min. After the reaction was completed as detected by LC-MS, the reaction solution was poured into water, followed by extraction with EA (10 mL×2). The organic phases were combined, dried and concentrated, and the crude product was purified by silica gel column chromatography (dichloromethane:methanol=200:1 to 100:1, v/v) to give the product (480 mg, crude product).

### Step 7: Synthesis of Intermediate (R)-3-hydroxy-4-(8-(piperidin-3-ylamino)pyrido[2,3-d]pyridazin-5-yl)benzonitrile

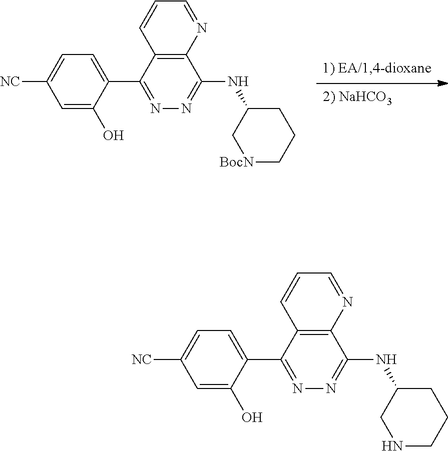

Tert-butyl (R)-3-((5-(4-cyano-2-hydroxyphenyl)pyrido[2,3-d]pyridazin-8-yl)amino)piperidine-1-carboxylate (1 mmol, 1.0 eq.) was dissolved in dichloromethane (2 mL), and the solution was added dropwise into a 4 mol/L solution of hydrogen chloride in 1,4-dioxane (4 mL). After the completion of dropwise addition, a large number of solids precipitated. The mixture was reacted at room temperature for 5 min. After the reaction was completed as detected by TLC, water (5 mL) was added to the reaction solution, the pH was adjusted to alkalinity with sodium bicarbonate, and the aqueous phase was concentrated to give the crude product. The crude product was dissolved in dichloromethane/methanol (20:1, 20 mL), and the solution was filtered. The filtrate was dried and concentrated to give the product (279 mg, two-step yield: 80.6%).

### Step 8: Synthesis of compound (R)-3-hydroxy-4-(8-((1-methylpiperidin-3-yl)amino)pyrido[2,3-d]pyridazin-5-yl)benzonitrile

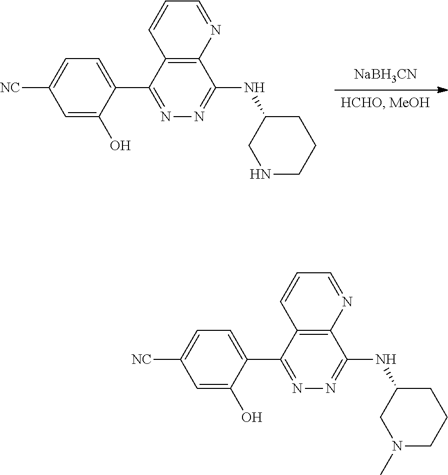

(R)-3-hydroxy-4-(8-(piperidin-3-ylamino)pyrido[2,3-d]pyridazin-5-yl)benzonitrile (279 mg, 0.832 mmol, 1.0 eq.) was dissolved in methanol (3 mL), followed by addition of an aqueous formaldehyde solution (37%) (68 mg, 0.832 mmol, 1.0 eq.). The mixture was stirred at room temperature for 5 min, followed by addition of sodium cyanoborohydride (58 mg, 0.915 mmol, 1.1 eq.). The resulting mixture was reacted at room temperature for 5 min. After the reaction was completed as detected by TLC, the reaction solution was concentrated, saturated brine (1 mL) was added, and the pH was adjusted to alkalinity with sodium bicarbonate, followed by extraction with a mixed solvent (dichloromethane:methanol:ammonia solution=20:1:0.5, 20 mL×3). The organic phases were combined, dried and concentrated, and the crude product was purified by silica gel column chromatography (dichloromethane:methanol=50:1 to 20:1, v/v) and lyophilized to give the product (112 mg, yield: 37.3%).

¹H-NMR (400 MHz, DMSO-d6) δ (ppm): 11.93 (brs, 1H), 9.14 (s, 1H), 8.92-8.90 (d, 1H), 8.20-8.18 (d, 1H), 7.93 (s, 1H), 7.61-7.60 (d, 1H), 7.38-7.31 (m, 2H), 4.43 (s, 1H), 3.11-3.09 (d, 1H), 2.75-2.73 (d, 1H), 2.24 (s, 3H), 1.99-1.76 (m, 4H), 1.63-1.45 (m, 2H).

Molecular formula: C₂₀H₂₀N₆O Precise molecular weight: 360.17 LC-MS (m/z)=361.11 [M+H]⁺

### Example 20: Synthesis of Compound (R)-2-(6-bromo-4-((1-methylpiperidin-3-yl)amino)phthalazin-1-yl)-5-methylphenol (Compound 60)

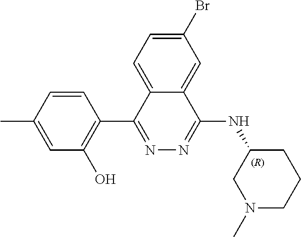

### Step 1: Synthesis of 2-methoxy-4-methylbenzoyl chloride

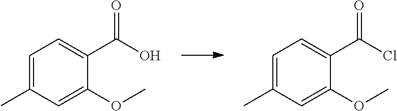

2-Methoxy-4-methylbenzoic acid (5.0 g, 30.08 mmol, 1.0 eq.) and DMF (0.1 mL) were dissolved in DCM (100 mL), followed by addition of oxalyl chloride (2.30 g, 18.05 mmol, 0.6 eq.). The mixture was reacted at room temperature for 15 h, and the reaction solution was concentrated under reduced pressure to give the product (5.55 g, yield: 100%).

### Step 2: Synthesis of methyl 5-bromo-2-(2-methoxy-4-methylbenzoyl)benzoate

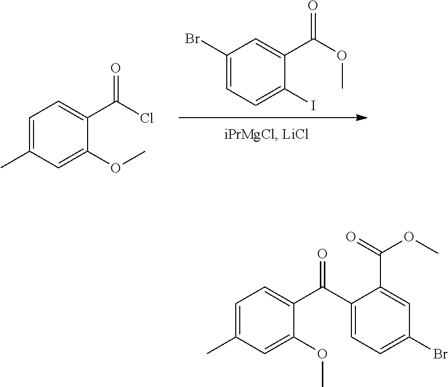

Methyl 5-bromo-2-iodobenzoate (10.3 g, 30.06 mmol, 1.0 eq.) was dissolved in THE (50 mL), and the mixture was cooled to −30° C., followed by dropwise and slow addition of a solution of an isopropylmagnesium chloride lithium chloride complex in tetrahydrofuran (1.3 mol/L, 25.4 mL, 33.07 mmol, 1.1 eq.). The resulting mixture was stirred for 30 min. The above reaction solution was added dropwise and slowly to a solution of 2-methoxy-4-methylbenzoyl chloride (5.55 g, 30.06 mmol, 1.0 eq.) in THE (50 mL) at −60° C., and the mixture was reacted at −70° C. After the completion of dropwise addition, the mixture was naturally warmed to room temperature and reacted for 2.5 h. The reaction solution was poured into water (100 mL), followed by extraction with EA (100 mL×2). The organic phase was dried and concentrated, and the crude product was purified by silica gel column chromatography (PE:EA=20:1) to give the product (3.5 g, yield: 32.1%).

### Step 3: Synthesis of 7-bromo-4-(2-methoxy-4-methylphenyl)phthalazin-1-ol

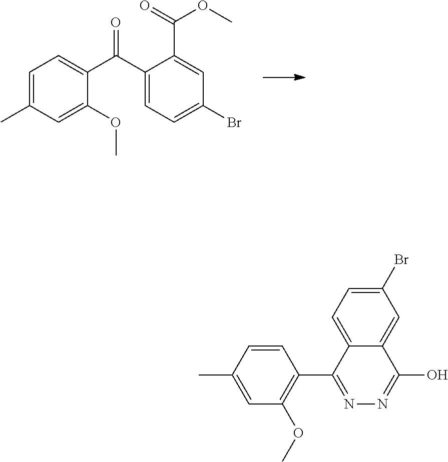

Methyl 5-bromo-2-(2-methoxy-4-methylbenzoyl)benzoate (3.5 g, 9.63 mmol, 1.0 eq.) and 85% hydrazine hydrate (0.85 g, 14.44 mmol, 1.5 eq.) were dissolved in ethanol (35 mL), and the mixture was reacted at 80° C. for 1.5 h. After the reaction was completed as monitored by TLC, the reaction solution was cooled to room temperature, and a large number of solids precipitated. The mixture was filtered under vacuum to give the product (1.8 g, yield: 54.2%).

### Step 4: Synthesis of 6-bromo-4-chloro-1-(2-methoxy-4-methylphenyl)phthalazine

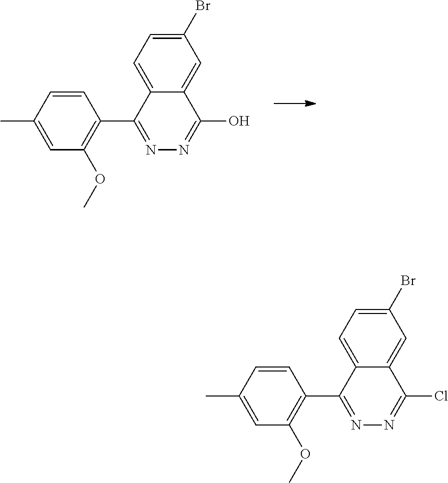 [SMILES: COc1cc(C)ccc1-c1nnc(O)c2cc(Br)ccc12 ; COc1cc(C)ccc1-c1nnc(Cl)c2cc(Br)ccc12]

7-Bromo-4-(2-methoxy-4-methylphenyl)phthalazin-1-ol (1.8 g, 5.21 mmol, 1.0 eq.) and phosphorus oxychloride (1.6 g, 10.42 mmol, 2.0 eq.) were dissolved in acetonitrile (20 mL), and the mixture was reacted at 90° C. for 2 h. After the reaction was completed as monitored by LC-MS, the reaction solution was concentrated under reduced pressure, and the crude product was dissolved in EA (30 mL), followed by addition of water (10 mL). The pH was adjusted to 9 with a saturated aqueous sodium bicarbonate solution, followed by liquid separation. The aqueous phase was extracted with EA (20 mL), and the organic phases were combined, dried and concentrated to give the product (1.6 g, yield: 84.6%).

### Step 5: Synthesis of tert-butyl (R)-3-((7-bromo-4-(2-methoxy-4-methylphenyl)phthalazin-1-yl)amino)piperidine-1-carboxylate

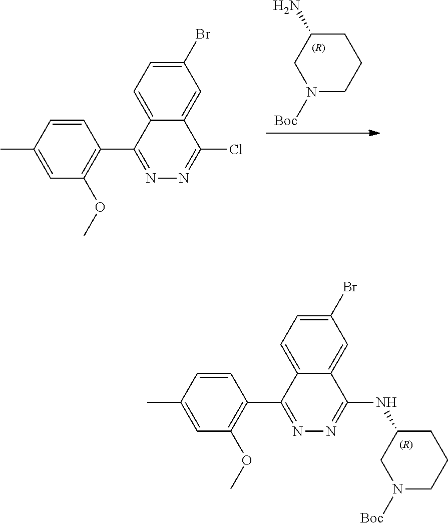

6-Bromo-4-chloro-1-(2-methoxy-4-methylphenyl)phthalazine (1.6 g, 4.40 mmol, 1.0 eq.) and tert-butyl (R)-3-aminopiperidine-1-carboxylate (1.76 g, 8.80 mmol, 2.0 eq.) were dissolved in DMAC (16 mL), and the mixture was reacted at 120° C. for 26 h. After the reaction was completed as detected by LC-MS, the reaction solution was poured into water (50 mL), and solids precipitated. The mixture was filtered under vacuum, and the filter cake was dissolved in EA (50 mL) and dried. The organic phase was concentrated under reduced pressure to give the product (1.2 g, yield: 51.7%).

### Step 6: Synthesis of (R)-2-(6-bromo-4-(piperidin-3-ylamino)phthalazin-1-yl)-5-methylphenol

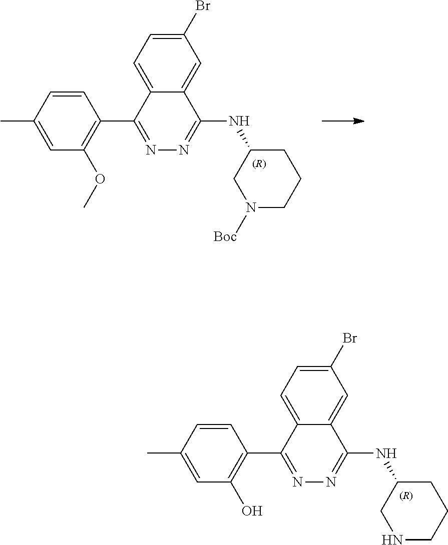

Tert-butyl (R)-3-((7-bromo-4-(2-methoxy-4-methylphenyl)phthalazin-1-yl)amino)piperidine-1-carboxylate (1.0 g, 1.89 mmol, 1.0 eq.) was dissolved in DCM (10 mL), and the mixture was cooled to −20° C., followed by dropwise addition of a 1 mol/L solution of boron tribromide in dichloromethane (5.7 mL, 5.67 mmol, 3.0 eq.). After the completion of dropwise addition, the resulting mixture was naturally warmed to room temperature and reacted for 1 h. After the reaction was completed as detected by LC-MS, an appropriate amount of methanol was added to the flask to quench the reaction. The reaction solution was concentrated under reduced pressure, and the crude product was dissolved in water (20 mL). EA (20 mL×2) was added for extraction, and the aqueous phase was retained. The pH was adjusted to 9 with NaHCO₃, followed by extraction with DCM (20 mL×2). The organic phases were combined, dried and concentrated to give the product (400 mg, yield: 51.2%).

### Step 7: Synthesis of (R)-2-(6-bromo-4-((1-methylpiperidin-3-yl)amino)phthalazin-1-yl)-5-methylphenol

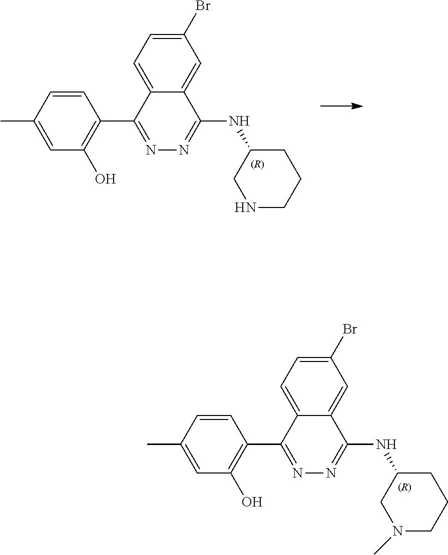

(R)-2-(6-bromo-4-(piperidin-3-ylamino)phthalazin-1-yl)-5-methylphenol (400 mg, 0.96 mmol, 1.0 eq.) was dissolved in methanol (4 mL), followed by addition of an aqueous formaldehyde solution (37%) (78 mg, 0.96 mmol, 1.0 eq.). The mixture was stirred at room temperature for 5 min, followed by addition of sodium cyanoborohydride (60 mg, 0.96 mmol, 1.0 eq.). The mixture was reacted at room temperature for 5 min. After the generation of the target product as monitored by LC-MS, the reaction solution was concentrated under reduced pressure, and the crude product was slurried with water (10 mL) and filtered under vacuum. The filter cake was purified by silica gel column chromatography (DCM:MeOH=30:1) to give the product (200 mg, yield: 48.8%).

¹HNMR (300 MHz, DMSO-d6) δ (ppm): 9.60 (s, 1H), 8.75 (s, 1H), 7.97-7.94 (d, 1H), 7.45-7.42 (d, 1H), 7.35 (s, 1H), 7.17-7.15 (d, 1H), 6.82-6.77 (t, 2H), 4.53 (s, 1H), 3.03 (s, 2H), 2.51 (s, 4H), 2.33 (s, 4H), 2.02-1.92 (d, 2H), 1.74-1.62 (m, 2H).

Molecular formula: C₂₁H₂₃BrN₄O Molecular weight: 427.35 LC-MS (Pos, m/z): 427.05/429.08[M+H]⁺.

### Example 21: Synthesis of Compound (R)-5-methyl-2-(7-((1-methylpiperidin-3-yl)amino)thieno[2,3-d]pyridazin-4-yl)phenol (Compound 70)

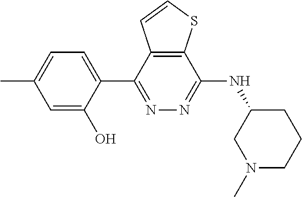 [SMILES: Cc1ccc(-c2nnc(N[C@@H]3CCCN(C)C3)c3sccc23)c(O)c1]

### Step 1: Synthesis of 7-chloro-4-(2-methoxy-4-methylphenyl)thieno[2,3-d]pyridazine

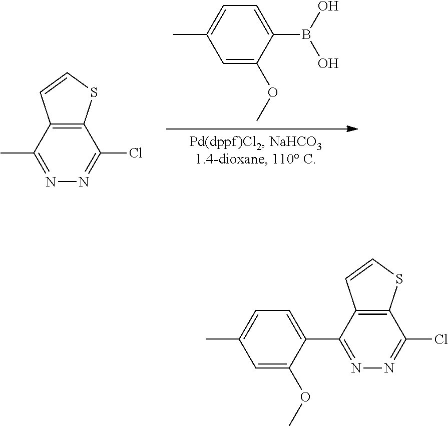

(2-Methoxy-4-methylphenyl)boronic acid (0.809 g, 4.88 mmol, 1.0 eq.), 4,7-dichlorothieno[2,3-d]pyridazine (1.00 g, 4.88 mmol, 1.0 eq.), Pd(dppf)Cl₂ (0.179 g, 0.244 mmol, 0.05 eq.) and NaHCO₃ (0.820 g, 9.76 mmol, 2.0 eq.) were added successively to 1,4-dioxane (20 mL), followed by addition of H₂O (10 mL). The mixture was heated to 110° C. and reacted for 2 h in nitrogen atmosphere. Water (60 mL) was added, and EA (50 mL) was added for extraction. The organic phase was dried over anhydrous sodium sulfate and filtered, and the filtrate was concentrated under reduced pressure. The crude product was separated by silica gel column chromatography (PE:EA=5:1 to 1:1) to give the product (820 mg, yield: 57.9%).

### Step 2: Synthesis of tert-butyl (R)-3-((4-(2-methoxy-4-methylphenyl)thieno[2,3-d]pyridazin-7-yl)amino)piperidine-1-carboxylate

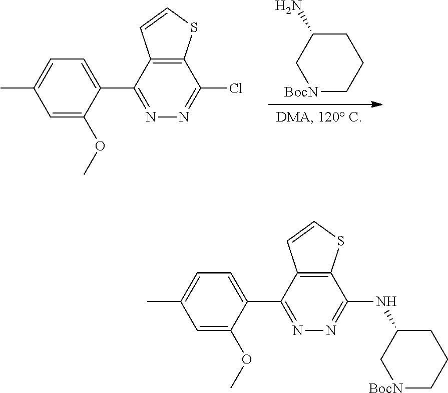

7-Chloro-4-(2-methoxy-4-methylphenyl)thieno[2,3-d]pyridazine (820 mg, 2.92 mmol, 1.0 eq.) and tert-butyl (R)-3-aminopiperidine-1-carboxylate (877 mg, 4.38 mmol, 1.5 eq.) were dissolved in DMA (10 mL), and the mixture was heated to 120° C. and reacted for 16 h in nitrogen atmosphere. Water (50 mL) was added to quench the reaction, and EA (50 mL) was added for extraction. The organic phase was washed with water (20 mL×3), dried over anhydrous sodium sulfate and filtered, and the filtrate was concentrated under reduced pressure. The crude product was separated by silica gel column chromatography (DCM:MeOH=100:1 to 50:1) to give the product (610 mg, yield: 47.6%).

### Step 3: Synthesis of (R)-5-methyl-2-(7-(piperidin-3-ylamino)thieno[2,3-d]pyridazin-4-yl)phenol

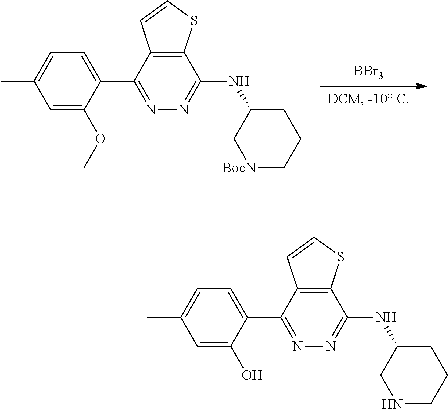 [SMILES: COc1cc(C)ccc1-c1nnc(N[C@@H]2CCCN(C(=O)OC(C)(C)C)C2)c2sccc12 ; "BBr3" ; "DCM, -10° C." ; Cc1ccc(-c2nnc(N[C@@H]3CCCNC3)c3sccc23)c(O)c1]

Tert-butyl (R)-3-((4-(2-methoxy-4-methylphenyl)thieno[2,3-d]pyridazin-7-yl)amino)piperidine-1-carboxylate (610 mg, 1.34 mmol, 1.0 eq.) was dissolved in DCM (12 mL), and the mixture was cooled to −10° C., followed by dropwise addition of BBr₃ (1.34 g, 5.36 mmol, 4.0 eq.). The mixture was reacted for 4 h. MeOH (5 mL) was added to quench the reaction, a saturated aqueous NaHCO₃ solution was added to adjust the pH to 8, and DCM (30 mL×2) was added for extraction. The organic phase was dried over anhydrous sodium sulfate and filtered, and the filtrate was concentrated under reduced pressure to give the product (380 mg, yield: 83.2%).

### Step 4: Synthesis of (R)-5-methyl-2-(7-((1-methylpiperidin-3-yl)amino)thieno[2,3-d]pyridazin-4-yl)phenol

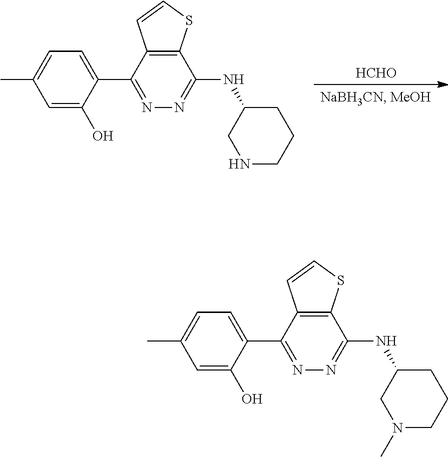

(R)-5-methyl-2-(7-(piperidin-3-ylamino)thieno[2,3-d]pyridazin-4-yl)phenol (380 mg, 1.12 mmol, 1.0 eq.) was dissolved in MeOH (4 mL), followed by addition of an aqueous formaldehyde solution (37%) (273 mg, 3.36 mmol, 3.0 eq.). The mixture was stirred at room temperature for 30 min. NaBH₃CN (211 mg, 3.36 mmol, 3.0 eq.) was then added and the mixture was reacted for 1 h. A saturated aqueous NaHCO₃ solution was added to quench the reaction, and DCM (30 mL×2) was added for extraction. The organic phase was dried over anhydrous sodium sulfate and filtered, and the filtrate was concentrated under reduced pressure. The crude product was separated by preparative thin-layer chromatography (DCM:MeOH=15:1) to give the product (70 mg, yield: 17.7%).

¹HNMR (400 MHz, DMSO-d6) δ (ppm): 11.43 (s, 1H), 8.12 (d, J=5.2 Hz, 1H), 7.56 (d, J=5.6 Hz, 1H), 7.52 (d, J=8.0 Hz, 1H), 7.07 (d, J=7.6 Hz, 1H), 6.82 (s, 1H), 6.79 (d, J=8.0 Hz, 1H), 4.40-4.38 (m, 1H), 3.09 (d, J=8.8 Hz, 1H), 2.75 (d, J=10.4 Hz, 1H), 2.32 (s, 3H), 2.26 (s, 3H), 1.99-1.96 (m, 3H), 1.77-1.74 (m, 1H), 1.66-1.57 (m, 1H), 1.50-1.41 (m, 1H).

Molecular formula: C₁₉H₂₂N₄OS Molecular weight: 354.47 LC-MS (Pos, m/z)=355.13[M+H]⁺.

### Example 22: Synthesis of Compound (R)-5-bromo-2-(4-((1-methylpiperidin-3-yl)amino)phthalazin-1-yl)phenol (Compound 73)

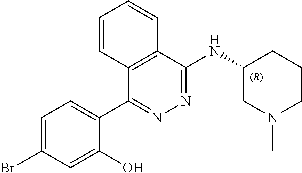

### Step 1: Synthesis of 2-(4-bromo-2-methoxybenzoyl)benzoic acid

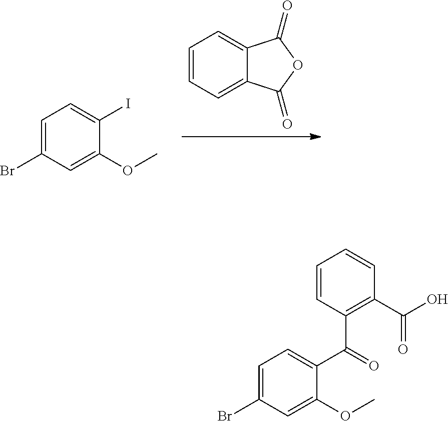

4-Bromo-1-iodo-2-methoxybenzene (5.0 g, 15.97 mmol, 1.0 eq.) was added to tetrahydrofuran (50.0 mL), and the mixture was cooled to −20° C. in nitrogen atmosphere, followed by dropwise addition of a solution of isopropylmagnesium chloride-lithium chloride in tetrahydrofuran (1.3 mol/L, 14.7 mL, 19.17 mmol, 1.2 eq.). The resulting mixture was reacted at −20° C. for 0.5 h, and then the reaction solution was added dropwise to a solution of phthalic anhydride (2.6 g, 17.56 mmol, 1.1 eq.) in tetrahydrofuran (20.0 mL) cooled to −20° C. The mixture was warmed to room temperature and reacted for 2 h. After the reaction was completed as monitored by TLC, ethyl acetate (200.0 mL) was added, followed by washing with a saturated aqueous ammonium chloride solution (100.0 mL). The organic phase was dried over anhydrous sodium sulfate and filtered, and the filtrate was concentrated under reduced pressure to give the product (5.0 g, yield: 93.4%).

### Step 2: Synthesis of methyl 2-(4-bromo-2-methoxybenzoyl)benzoate

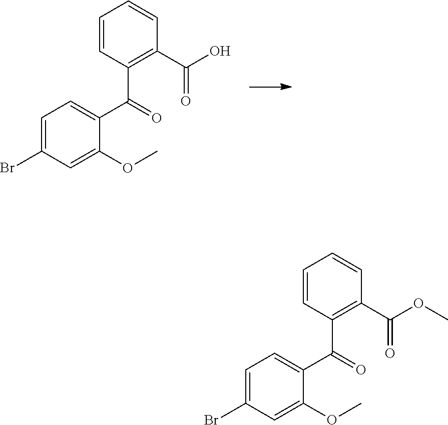 [SMILES: COc1cc(Br)ccc1C(=O)c1ccccc1C(=O)O ; COC(=O)c1ccccc1C(=O)c1ccc(Br)cc1OC]

2-(4-Bromo-2-methoxybenzoyl)benzoic acid (5.0 g, 14.91 mmol, 1.0 eq.), potassium carbonate (3.0 g, 22.36 mmol, 1.5 eq.) and iodomethane (2.7 g, 19.39 mmol, 1.3 eq.) were added to N,N-dimethylformamide (30.0 mL), and the mixture was reacted at room temperature for 4 h. After the reaction was completed as monitored by TLC, ethyl acetate (200.0 mL) was added, followed by washing with water (100.0 mL×3). The organic phase was dried over anhydrous sodium sulfate and filtered, and the filtrate was concentrated under reduced pressure. The crude product was purified by silica gel column chromatography (specification of silica gel: 100-200 mesh, petroleum ether:ethyl acetate=30:1 to 10:1) to give the product (3.68 g, yield: 70.7%).

### Step 3: Synthesis of 4-(4-bromo-2-methoxyphenyl)phthalazin-1-ol

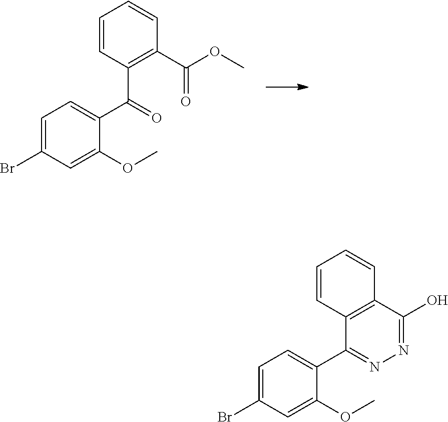 [SMILES: COC(=O)c1ccccc1C(=O)c1ccc(Br)cc1OC ; COc1cc(Br)ccc1-c1nnc(O)c2ccccc12]

Methyl 2-(4-bromo-2-methoxybenzoyl)benzoate (2.0 g, 5.72 mmol, 1.0 eq.) was added to ethanol (20.0 mL) and hydrazine hydrate (85%) (505.9 mg, 8.59 mmol, 1.5 eq.), and the mixture was reacted at room temperature for 1 h. After the reaction was completed as monitored by LC-MS, the reaction solution was filtered, and the filter cake was washed with ethanol (50.0 mL) and dried to give the product (1.25 g, yield: 66.1%).

### Step 4: Synthesis of 1-(4-bromo-2-methoxyphenyl)-4-chlorophthalazine

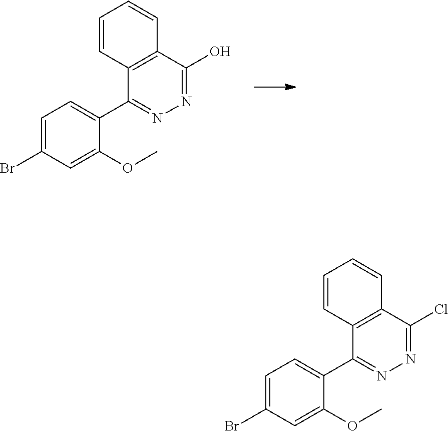 [SMILES: COc1cc(Br)ccc1-c1nnc(O)c2ccccc12 ; COc1cc(Br)ccc1-c1nnc(Cl)c2ccccc12]

4-(4-Bromo-2-methoxyphenyl)phthalazin-1-ol (1.25 g, 3.77 mmol, 1.0 eq.) and phosphorus oxychloride (3.47 g, 22.64 mmol, 6.0 eq.) were added to acetonitrile (20.0 mL), and the mixture was refluxed for 1 h. After the reaction was completed as monitored by TLC, the reaction solution was poured into ice water (200.0 mL), and ethyl acetate (100.0 mL) was added for extraction. The organic phase was dried over anhydrous sodium sulfate and filtered, and the filtrate was concentrated under reduced pressure to give the product (1.1 g, yield: 83.9%).

### Step 5: Synthesis of tert-butyl (R)-3-((4-(4-bromo-2-methoxyphenyl)phthalazin-1-yl)amino)piperidine-1-carboxylate

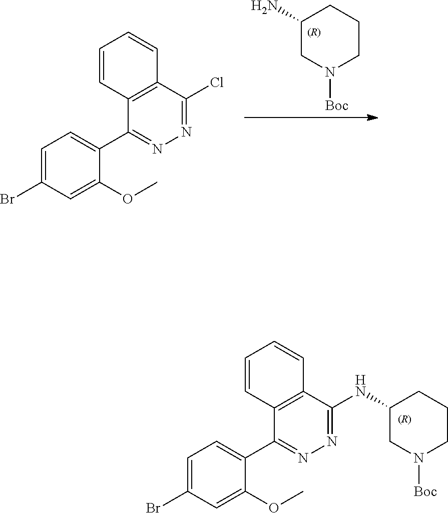

1-(4-Bromo-2-methoxyphenyl)-4-chlorophthalazine (1.1 g, 3.14 mmol, 1.0 eq.) and tert-butyl (R)-3-aminopiperidine-1-carboxylate (1.26 g, 6.29 mmol, 2.0 eq.) were added to N,N-dimethylacetamide (20.0 mL), and the mixture was stirred at 120° C. for 12 h. After the reaction was completed as monitored by TLC, the reaction solution was cooled to room temperature, and ethyl acetate (100.0 mL) was added, followed by washing with water (100.0 mL). The organic phase was dried over anhydrous sodium sulfate and filtered, and the filtrate was concentrated under reduced pressure. The crude product was purified by silica gel column chromatography (specification of silica gel: 100-200 mesh, petroleum ether:ethyl acetate=4:1 to 1:1) to give the product (920.0 mg, yield: 57.1%).

### Step 6: Synthesis of (R)-4-(4-bromo-2-methoxyphenyl)-N-(piperidin-3-yl)phthalazin-1-amine

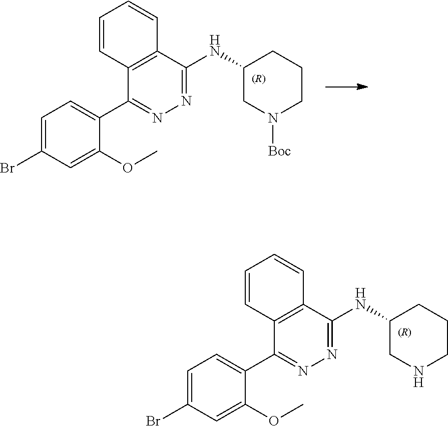

Tert-butyl (R)-3-((4-(4-bromo-2-methoxyphenyl)phthalazin-1-yl)amino)piperidine-1-carboxylate (920.0 mg, 1.79 mmol, 1.0 eq.) was added to dichloromethane (6.0 mL) and trifluoroacetic acid (6.0 mL), and the mixture was stirred at room temperature for 1 h. After the reaction was completed as monitored by TLC, the pH was adjusted to 7-8 with a saturated aqueous sodium bicarbonate solution, followed by extraction with dichloromethane (100.0 mL). The organic phase was dried over anhydrous sodium sulfate and filtered, and the filtrate was concentrated under reduced pressure to give the product (720 mg, yield: 97.3%).

### Step 7: Synthesis of (R)-4-(4-bromo-2-methoxyphenyl)-N-(1-methylpiperidin-3-yl)phthalazin-1-amine

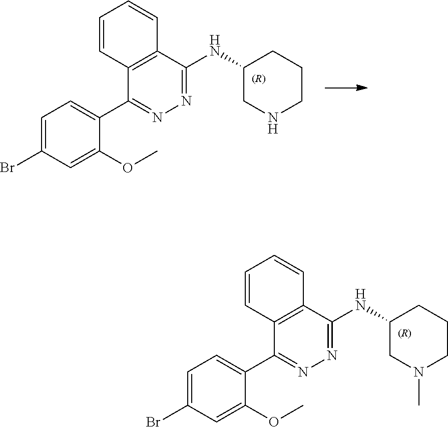

(R)-4-(4-bromo-2-methoxyphenyl)-N-(piperidin-3-yl) phthalazin-1-amine (720.0 mg, 1.74 mmol, 1.0 eq.) and an aqueous formaldehyde solution (37%) (169.6 mg, 2.09 mmol, 1.2 eq.) were added to methanol (8.0 mL), and the mixture was stirred at room temperature for 0.5 h, followed by addition of sodium cyanoborohydride (153.0 mg, 2.43 mmol, 1.4 eq.). The resulting mixture was reacted at room temperature for 1 h. After the reaction was completed as monitored by TLC, a saturated aqueous sodium bicarbonate solution (50.0 mL) was added, and the mixture was stirred for 30 min, followed by adjustment of the pH to 7-8 and extraction with dichloromethane (100.0 mL). The organic phase was dried over anhydrous sodium sulfate and filtered, and the filtrate was concentrated under reduced pressure. The crude product was purified by silica gel column chromatography (dichloromethane:methanol=40:1 to 10:1) to give the product (520.0 mg, yield: 69.9%).

### Step 8: Synthesis of (R)-5-bromo-2-(4-((1-methylpiperidin-3-yl)amino)phthalazin-1-yl)phenol

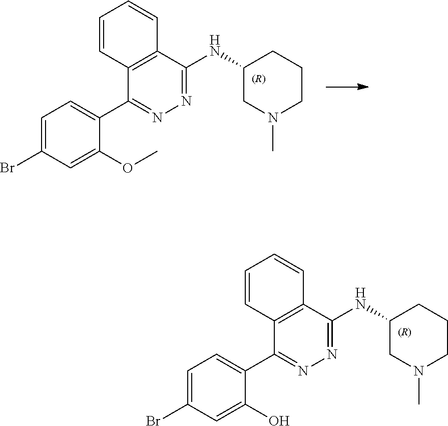

(R)-4-(4-bromo-2-methoxyphenyl)-N-(1-methylpiperidin-3-yl)phthalazin-1-amine (260.0 mg, 0.60 mmol, 1.0 eq.) was added to dichloromethane (5.0 mL), and the mixture was cooled to −20° C. in nitrogen atmosphere, followed by dropwise addition of boron tribromide (300.6 mg, 1.20 mmol, 2.0 eq.). The resulting mixture was gradually warmed to room temperature and reacted for 2 h. After the reaction was completed as monitored by TLC, methanol (5.0 mL) was added dropwise and slowly under an ice bath to quench the reaction, and the mixture was concentrated under reduced pressure, followed by addition of a saturated aqueous sodium bicarbonate solution (50.0 mL). The resulting mixture was stirred for 30 min, and the pH was adjusted to 7-8, followed by extraction with dichloromethane (50.0 mL). The organic phase was dried over anhydrous sodium sulfate and filtered, and the filtrate was concentrated under reduced pressure. The crude product was purified by preparative thin-layer chromatography to give the product (90.0 mg, yield 36.2%).

¹HNMR (300 MHz, DMSO-d6) δ (ppm): 10.15 (s, 1H), 8.40-8.37 (d, J=9 Hz, 1H), 7.87-7.75 (m, 2H), 7.48-7.45 (d, J=9 Hz, 1H), 7.26-7.23 (d, J=9 Hz, 1H), 7.17-7.07 (m, 3H), 4.41 (s, 1H), 3.09-3.07 (d, J=6 Hz, 1H), 2.73-2.69 (d, J=12 Hz, 1H), 2.22 (s, 3H), 1.99-1.89 (m, 3H), 1.78-1.43 (m, 3H).

Molecular formula: C₂₀H₂₁BrN₄O Precise molecular weight: 412.09 LC-MS (m/z)=413.01/415.03[M+H]⁺.

### Example 23: Synthesis of Compound (R)-5-methoxy-2-(4-((1-methylpiperidin-3-yl)amino)phthalazin-1-yl)phenol (Compound 74)

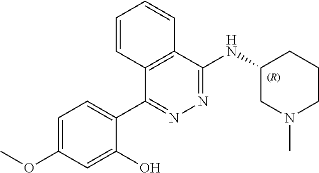

### Step 1: Synthesis of 1-bromo-2-(ethoxymethyl)-4-methoxybenzene

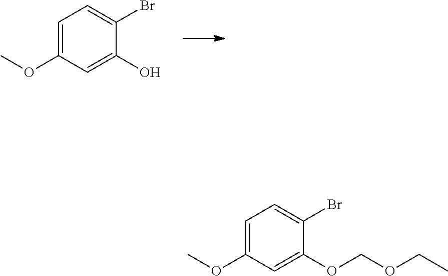

2-Bromo-5-methoxyphenol (1.8 g, 8.86 mmol, 1.0 eq.) was added into dry tetrahydrofuran (20.0 mL), and the mixture was cooled to 0° C. in nitrogen atmosphere, followed by slow addition of sodium hydride (60%) (531.6 mg, 13.29 mmol, 1.5 eq.). The resulting mixture was reacted for 0.5 h, followed by addition of chloromethoxy ethane (1.17 g, 12.40 mmol, 1.4 eq.). The mixture was warmed to room temperature and reacted for 12 h. After the reaction was completed as monitored by TLC, water (20.0 mL) was added to the system, followed by extraction with ethyl acetate (100.0 mL). The organic phase was dried over anhydrous sodium sulfate and filtered, and the filtrate was concentrated under reduced pressure. The crude product was purified by silica gel column chromatography (specification of silica gel: 100-200 mesh, petroleum ether:ethyl acetate=10:1) to give the product (2.1 g, yield: 91.3%).

### Step 2: Synthesis of 2-(2-(ethoxymethoxy)-4-methoxyphenyl)-4,4,5,5-tetramethyl-1,3,2-dioxaborolane

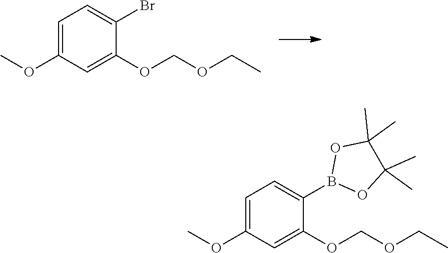 [SMILES: CCOCOc1cc(OC)ccc1Br ; CCOCOc1cc(OC)ccc1B1OC(C)(C)C(C)(C)O1]

1-Bromo-2-(ethoxymethyl)-4-methoxybenzene (2.1 g, 8.04 mmol, 1.0 eq.), bis(pinacolato)diboron (3.06 g, 12.06 mmol, 1.5 eq.), potassium acetate (1.57 g, 16.08 mmol, 2.0 eq.) and Pd(dppf)Cl₂ (294.0 mg, 0.40 mmol, 0.05 eq.) were added to 1,4-dioxane (20.0 mL), followed by purge with nitrogen for 5 min. The mixture was reacted at 100° C. for 2 h. After the reaction was completed as monitored by TLC, the reaction solution was purified by silica gel column chromatography (specification of silica gel: 100-200 mesh, petroleum ether: ethyl acetate=10:1) to give the product (2.1 g, yield: 87.5%).

### Step 3: Synthesis of (R)-4-(2-(ethoxymethoxy)-4-methoxyphenyl)-N-(1-methylpiperidin-3-yl)phthalazin-1-amine

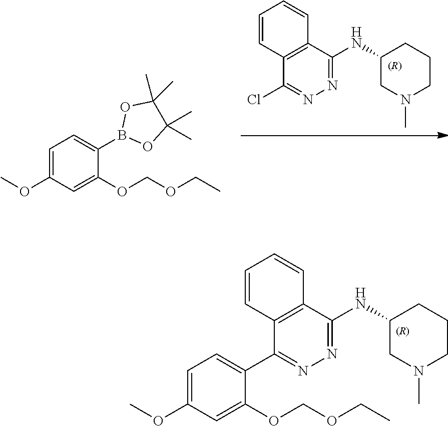

(R)-4-chloro-N-(1-methylpiperidin-3-yl)phthalazin-1-amine (505.0 mg, 1.82 mmol, 1.0 eq.), 2-(2-(ethoxymethoxy)-4-methoxyphenyl)-4,4,5,5-tetramethyl-1,3,2-dioxaborolane (843.4 mg, 2.73 mmol, 1.5 eq.), sodium bicarbonate (305.7 mg, 3.64 mmol, 2.0 eq.), water (5.0 mL) and tetrakis(triphenylphosphine)palladium (105.1 mg, 0.09 mmol, 0.05 eq.) were added to 1,4-dioxane (10.0 mL), and the mixture was reacted at 110° C. for 4 h in nitrogen atmosphere. After the reaction was completed as monitored by TLC, water (100.0 mL) was added, followed by extraction with ethyl acetate (100.0 mL). The organic phase was dried and filtered, and the filtrate was concentrated under reduced pressure. The crude product was purified by silica gel column chromatography (specification of silica gel: 100-200 mesh, dichloromethane:methanol=40:1 to 10:1) to give the product (470.0 mg, yield: 61.1%).

### Step 4: Synthesis of (R)-5-methoxy-2-(4-((1-methylpiperidin-3-yl)amino)phthalazin-1-yl)phenol

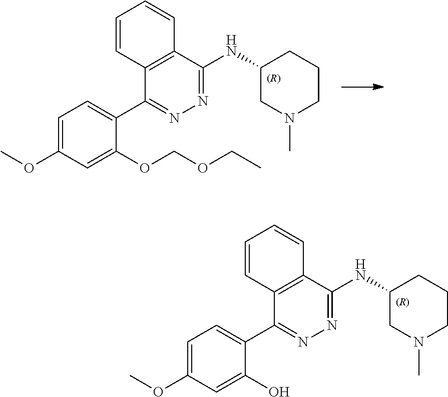

(R)-4-(2-(ethoxymethoxy)-4-methoxyphenyl)-N-(1-methylpiperidin-3-yl)phthalazin-1-amine (470.0 mg, 1.11 mmol, 1.0 eq.) was added to dichloromethane (5.0 mL), followed by dropwise addition of trifluoroacetic acid (3.0 mL). The mixture was reacted at room temperature for 2 h. After the reaction was completed as monitored by TLC, the pH was adjusted to 7-8 with a saturated aqueous sodium bicarbonate solution under an ice bath, followed by extraction with dichloromethane (50.0 mL). The organic phase was dried over anhydrous sodium sulfate and filtered, and the filtrate was concentrated under reduced pressure. The crude product was purified by preparative thin-layer chromatography (dichloromethane:methanol=10:1) to give the product (50.0 mg, yield: 12.3%).

¹HNMR (300 MHz, DMSO-d6) δ (ppm): 9.90 (s, 1H), 8.47 (s, 1H), 7.77-7.88 (m, 2H), 7.55-7.58 (m, 1H), 7.36 (s, 1H), 7.20-7.23 (d, J=6 Hz, 1H), 6.54-6.59 (m, 2H), 4.61 (s, 1H), 3.79 (s, 3H), 3.36 (m, 1H), 3.05 (m, 2H), 2.58 (s, 3H), 1.74-2.04 (m, 5H).

Molecular formula: C₂₁H₂₄N₄O₂ Molecular weight: 364.45 LC-MS (Pos, m/z): 365.15[M+H]⁺.

### Example 24: Synthesis of Compound (R)-3-hydroxy-4-(4-((1-methylpiperidin-3-yl)amino)pyrido[3,4-d]pyridazin-1-yl)benzonitrile (Compound 79)

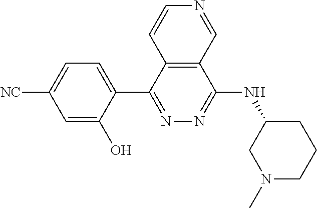 [SMILES: CN1CCC[C@@H](Nc2nnc(-c3ccc(C#N)cc3O)c3ccncc23)C1]

### Step 1: Synthesis of Intermediate 4-(4-bromo-2-methoxybenzoyl)nicotinic acid

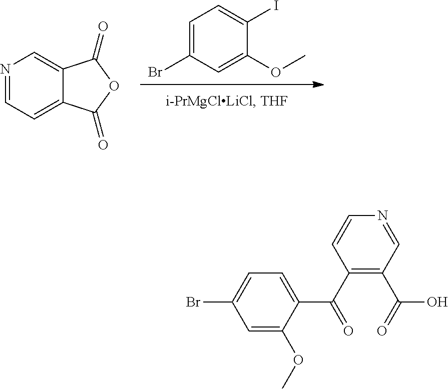

4-Bromo-1-iodo-2-methoxybenzene (21 g, 67.06 mmol, 1.0 eq.) was dissolved in anhydrous THE (200 mL), followed by dropwise addition of a solution of i-PrMgCl LiCl in THE (1.3 mol/L, 55 mL, 70.41 mmol, 1.05 eq.) at −20° C. After the completion of dropwise addition, the mixture was reacted for 30 min. At −20° C., the above reaction solution was added dropwise to a solution of 3,4-pyridine dicarboxylic anhydride (10 g, 67.06 mmol, 1.0 eq.) in THE (100 mL). After the completion of dropwise addition, the mixture was reacted at room temperature for 1 h. After the reaction was completed as detected by LC-MS, the reaction solution was poured into a saturated aqueous ammonium chloride solution (200 mL), followed by concentration. A large number of solids precipitated, and the mixture was filtered. The filter cake was dried to give an isomer-containing mixed product (18.67 g, yield: 82.8%).

### Step 2: Synthesis of Intermediate 1-(4-bromo-2-methoxyphenyl)pyrido[3,4-d]pyridazin-4-ol

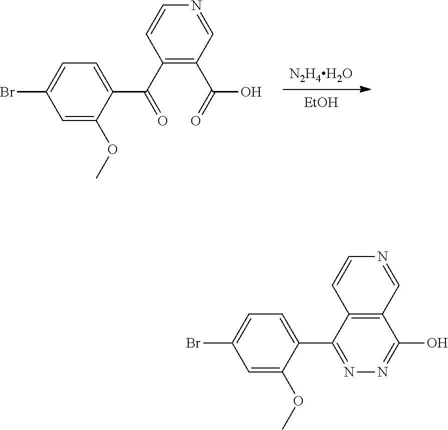 [SMILES: "EtOH" ; COc1cc(Br)ccc1C(=O)c1ccncc1C(=O)O ; "N2H4·H2O" ; COc1cc(Br)ccc1-c1nnc(O)c2cnccc12]

The mixture obtained in the previous step (5 g, 14.87 mmol, 1.0 eq.) was added to EtOH (30 mL), followed by addition of 85% hydrazine hydrate (1.76 g, 29.74 mmol, 2.0 eq.). The mixture was heated to 90° C. and reacted for 2 h. After the reaction was completed as detected by TLC, the reaction solution was concentrated, and the crude product was purified by silica gel column chromatography (dichloromethane:methanol=1200:1 to 300:1, v/v) to give the product (3.6 g, yield: 72.8%).

### Step 3: Synthesis of Intermediate 1-(4-bromo-2-methoxyphenyl)-4-chloropyrido[3,4-d]pyridazine

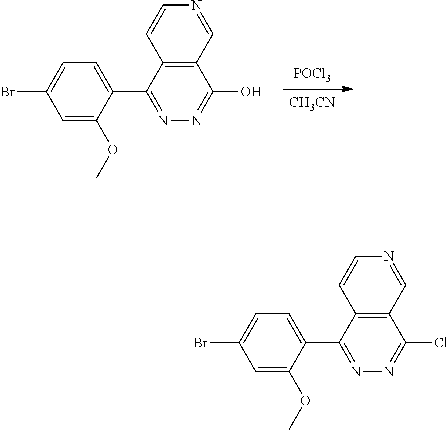

1-(4-Bromo-2-methoxyphenyl)pyrido[3,4-d]pyridazin-4-ol (3 g, 9.03 mmol, 1.0 eq.) was added to acetonitrile (50 mL), followed by addition of phosphorus oxychloride (2.77 g, 18.06 mmol, 2.0 eq.). The mixture was reacted at 100° C. for 16 h. After the reaction was completed as detected by TLC, the reaction solution was concentrated, and the concentrated solution was poured into ice water (10 mL). The pH was adjusted to alkalinity with sodium bicarbonate, followed by extraction with dichloromethane (30 mL×3). The organic phases were combined, dried and concentrated, and the crude product was purified by silica gel column chromatography (petroleum ether:ethyl acetate=10:1 to 5:1, v/v) to give the product (2.1 g, yield: 66%).

### Step 4: Synthesis of Intermediate 5-bromo-2-(4-chloropyrido[3,4-d]pyridazin-1-yl)phenol

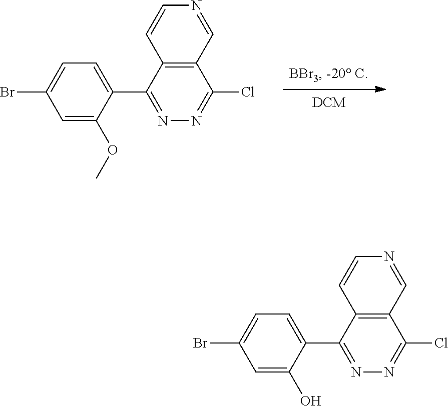

1-(4-Bromo-2-methoxyphenyl)-4-chloropyrido[3,4-d]pyridazine (2.1 g, 5.99 mmol, 1.0 eq.) was dissolved in dichloromethane (20 mL), followed by dropwise and slow addition of a solution of boron tribromide in n-heptane (1 mol/L, 9 mL, 8.96 mmol, 1.5 eq.) at −20° C. After the completion of dropwise addition, the mixture was reacted at room temperature for 10 min. After the reaction was completed as detected by TLC, a saturated sodium bicarbonate solution (10 mL) was added dropwise and slowly to the reaction solution under an ice bath, followed by extraction with dichloromethane (20 mL×3). The organic phases were combined, dried and concentrated, and the crude product was purified by silica gel column chromatography (petroleum ether:ethyl acetate=2:1, v/v) to give the product (1.52 g, yield: 76%).

### Step 5: Synthesis of Intermediate tert-butyl (R)-3-((1-(4-bromo-2-methoxyphenyl)pyrido[3,4-d]pyridazin-4-yl)amino)piperidine-1-carboxylate

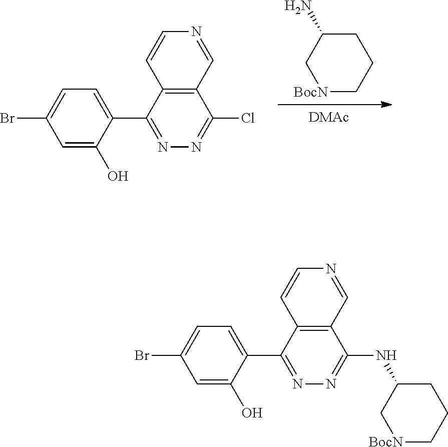

5-Bromo-2-(4-chloropyrido[3,4-d]pyridazin-1-yl)phenol (520 mg, 1.55 mmol, 1.0 eq.) was dissolved in DMAc (5 mL), followed by addition of (R)-1-tert-butoxycarbonyl-3-aminopiperidine (621 mg, 3.1 mmol, 2.0 eq.). The mixture was refluxed at 120° C. for 1 h. After the reaction was completed as detected by TLC, the reaction solution was added dropwise into water (5 mL), followed by extraction with ethyl acetate (10 mL×2). The organic phases were combined, dried and concentrated, and the crude product was purified by silica gel column chromatography (dichloromethane:methanol=200:1 to 50:1) to give the product (500 mg, yield: 64.5%).

### Step 6: Synthesis of Intermediate tert-butyl (R)-3-((1-(4-cyano-2-hydroxyphenyl)pyrido[3,4-d]pyridazin-4-yl)amino)piperidine-1-carboxylate

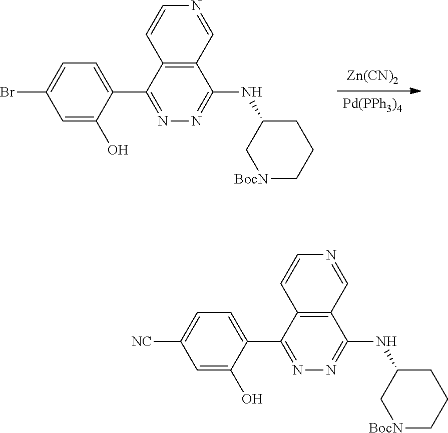 [SMILES: CC(C)(C)OC(=O)N1CCC[C@@H](Nc2nnc(-c3ccc(Br)cc3O)c3ccncc23)C1 ; "Zn(CN)2" ; CC(C)(C)OC(=O)N1CCC[C@@H](Nc2nnc(-c3ccc(C#N)cc3O)c3ccncc23)C1 ; "Pd(PPh3)4"]

Tert-butyl (R)-3-((1-(4-bromo-2-methoxyphenyl)pyrido[3,4-d]pyridazin-4-yl)amino)piperidine-1-carboxylate (500 mg, 1 mmol, 1.0 eq.) was dissolved in DMAc (5 mL), followed by addition of zinc cyanide (94 mg, 0.8 mmol, 0.8 eq.) and tetrakis(triphenylphosphine)palladium (112 mg, 0.1 mmol, 0.1 eq.) in nitrogen atmosphere. The mixture was reacted at 120° C. for 45 min. After the reaction was completed as detected by LC-MS, the reaction solution was poured into water, followed by extraction with EA (10 mL×2). The organic phases were combined, dried and concentrated, and the crude product was purified by silica gel column chromatography (dichloromethane:methanol=200:1, v/v) to give the product (350 mg, yield: 78.5%).

### Step 7: Synthesis of Intermediate (R)-3-hydroxy-4-(4-(piperidin-3-ylamino)pyrido[3,4-d]pyridazin-1-yl)benzonitrile

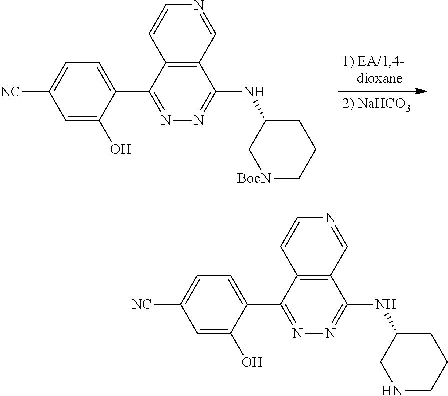

Tert-butyl (R)-3-((1-(4-cyano-2-hydroxyphenyl)pyrido[3,4-d]pyridazin-4-yl)amino)piperidine-1-carboxylate (350 mg, 0.784 mmol, 1.0 eq.) was dissolved in EA (1 mL), and the solution was added dropwise into a 4 mol/L solution of hydrogen chloride in 1,4-dioxane (4 mL). After the completion of dropwise addition, a large number of solids precipitated. The mixture was reacted at room temperature for 5 min. After the reaction was completed as detected by TLC, water (2 mL) was added to the reaction solution, the pH was adjusted to alkalinity with sodium bicarbonate, and the aqueous phase was concentrated to give the crude product. The crude product was dissolved in dichloromethane/methanol (20:1, 20 mL), and the solution was filtered. The filtrate was dried and concentrated, and the crude product was directly used in the next step.

### Step 8: Synthesis of compound (R)-3-hydroxy-4-(4-((1-methylpiperidin-3-yl)amino)pyrido[3,4-d]pyridazin-1-yl)benzonitrile

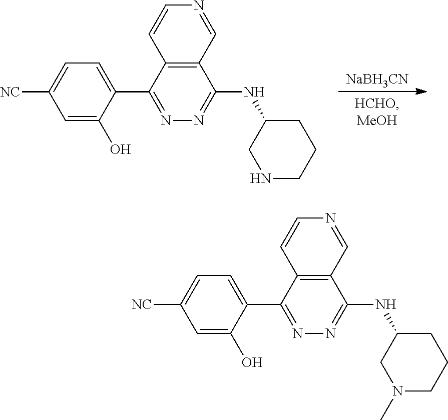

(R)-3-hydroxy-4-(4-(piperidin-3-ylamino)pyrido[3,4-d]pyridazin-1-yl)benzonitrile (0.783 mmol, 1.0 eq.) was dissolved in methanol (2 mL), followed by addition of an aqueous formaldehyde solution (37%) (63 mg, 0.783 mmol, 1.0 eq.). The mixture was stirred at room temperature for 5 min, followed by addition of sodium cyanoborohydride (55 mg, 0.862 mmol, 1.1 eq.). The resulting mixture was reacted at room temperature for 5 min. After the reaction was completed as detected by TLC, the reaction solution was concentrated, saturated brine (1 mL) was added, and the pH was adjusted to alkalinity with sodium bicarbonate, followed by extraction with a mixed solvent (dichloromethane:methanol:ammonia solution=20:1:0.5, 20 mL×2). The organic phases were combined, dried and concentrated, and the crude product was purified by silica gel column chromatography (dichloromethane:methanol=50:1 to 10:1, v/v) and lyophilized to give the product (91 mg, two-step yield: 32.2%).

¹H-NMR (400 MHz, DMSO-d6) δ (ppm): 10.62 (brs, 1H), 9.84 (s, 1H), 8.87-8.86 (d, 1H), 7.83 (s, 1H), 7.54-7.52 (d, 1H), 7.45-7.43 (m, 1H), 7.38-7.37 (d, 1H), 7.28-7.26 (d, 1H), 4.55 (s, 1H), 2.40 (s, 2H), 2.24 (m, 2H), 2.02 (s, 3H), 1.85 (m, 2H), 1.67 (m, 2H).

Molecular formula: C₂₀H₂₀N₆O Precise molecular weight: 360.17 LC-MS (m/z)=361.10 [M+H]⁺

### Example 25: Synthesis of Compound (R)-5-(difluoromethyl)-2-(4-((1-methylpiperidin-3-yl)amino)phthalazin-1-yl)phenol (Compound 87)

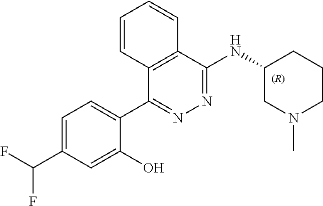

### Step 1: Synthesis of 3-methoxy-4-(4,4,5,5-tetramethyl-1,3,2-dioxaborolan-2-yl)benzaldehyde

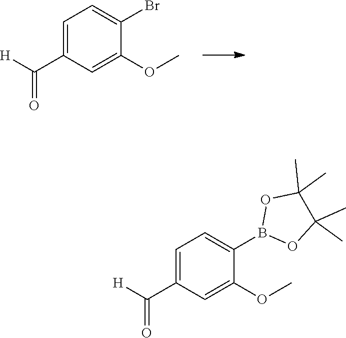 [SMILES: COc1cc(C=O)ccc1Br ; COc1cc(C=O)ccc1B1OC(C)(C)C(C)(C)O1]

4-Bromo-3-methoxybenzaldehyde (5.0 g, 23.25 mmol, 1.0 eq.), bis(pinacolato)diboron (8.8 g, 34.87 mmol, 1.5 eq.), potassium acetate (4.5 g, 46.50 mmol, 2.0 eq.) and Pd(dppf)Cl₂ (850.6 mg, 1.16 mmol, 0.05 eq.) were added to 1,4-dioxane (30.0 mL), and the mixture was reacted at 100° C. for 4 h in nitrogen atmosphere. After the reaction was completed as monitored by TLC, the crude product was purified by silica gel column chromatography (specification of silica gel: 100-200 mesh, petroleum ether:ethyl acetate=5:1) to give the product (5.5 g, yield: 91.6%).

### Step 2: Synthesis of (R)-3-methoxy-4-(4-((1-methylpiperidin-3-yl)amino)phthalazin-1-yl)benzaldehyde

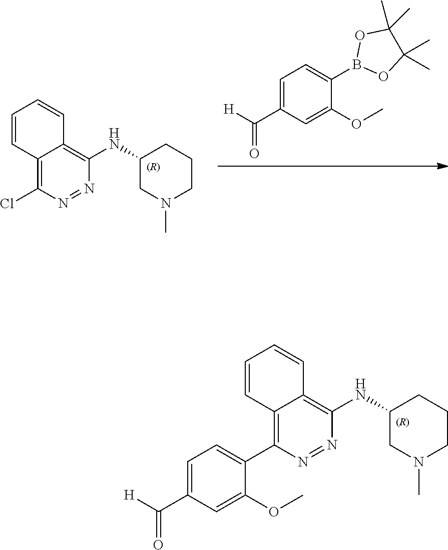

(R)-4-chloro-N-(1-methylpiperidin-3-yl)phthalazin-1-amine (443.7 mg, 1.60 mmol, 1.0 eq.), 3-methoxy-4-(4,4,5,5-tetramethyl-1,3,2-dioxaborolan-2-yl)benzaldehyde (630.3 mg, 2.40 mmol, 1.5 eq.), sodium bicarbonate (268.8 mg, 3.20 mmol, 2.0 eq.) and tetrakis(triphenylphosphine)palladium (92.4 mg, 0.08 mmol, 0.05 eq.) were added to 1,4-dioxane (10.0 mL) and water (5.0 mL), and the mixture was reacted at 110° C. for 4 h in nitrogen atmosphere. After the reaction was completed as monitored by TLC, ethyl acetate (200.0 mL) was added, followed by washing with a saturated aqueous sodium chloride solution (100.0 mL). The organic phase was dried over anhydrous sodium sulfate and filtered, and the filtrate was concentrated under reduced pressure. The crude product was purified by silica gel column chromatography (specification of silica gel: 100-200 mesh, dichloromethane:methanol=40:1 to 10:1) to give the product (410.0 mg, yield: 68.1%).

### Step 3: Synthesis of (R)-3-hydroxy-4-(4-((1-methylpiperidin-3-yl)amino)phthalazin-1-yl)benzaldehyde

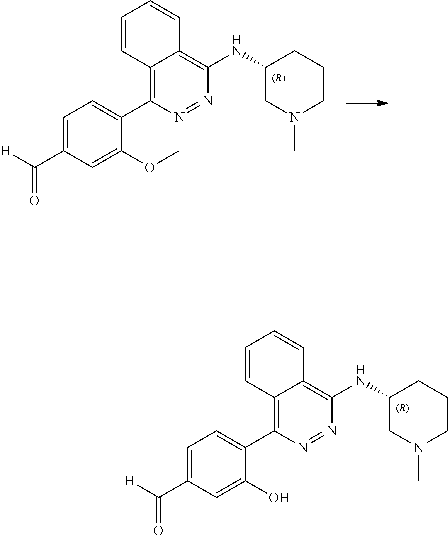

(R)-3-methoxy-4-(4-((1-methylpiperidin-3-yl)amino)phthalazin-1-yl)benzaldehyde (410.0 mg, 1.08 mmol, 1.0 eq.) was added to dichloromethane (10.0 mL), followed by dropwise addition of boron tribromide (818.5 mg, 3.26 mmol, 3.0 eq.) under an ice bath. The mixture was warmed to room temperature and stirred for 4 h. After the reaction was completed as monitored by TLC, methanol (5.0 mL) was added dropwise under an ice bath to quench the reaction, followed by concentration under reduced pressure, addition of dichloromethane (100.0 mL) and back extraction with water (50.0 mL). The pH of the aqueous phase was adjusted to 7-8 with a saturated aqueous sodium bicarbonate solution, followed by extraction with dichloromethane (100.0 mL). The organic phase was dried over anhydrous sodium sulfate and filtered, and the filtrate was concentrated under reduced pressure. The crude product was purified by preparative thin-layer chromatography to give the product (260.0 mg, yield: 66.4%).

### Step 4: Synthesis of (R)-5-(difluoromethyl)-2-(4-((1-methylpiperidin-3-yl)amino)phthalazin-1-yl)phenol

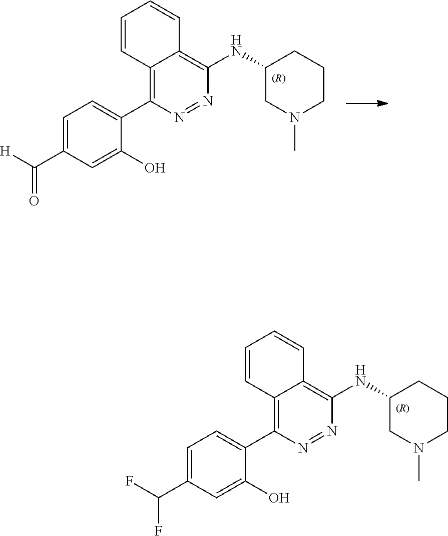

(R)-3-hydroxy-4-(4-((1-methylpiperidin-3-yl)amino)phthalazin-1-yl)benzaldehyde (260.0 mg, 0.71 mmol, 1.0 eq.) and diethylaminosulfur trifluoride (1.15 g, 7.1 mmol, 10.0 eq.) were added to dichloromethane (10.0 mL), and the mixture was stirred at room temperature for 12 h. After the reaction was completed as monitored by TLC, the reaction solution was added dropwise and slowly to ice water, and the pH was adjusted to 7-8 with a saturated aqueous sodium bicarbonate solution, followed by extraction with dichloromethane (100.0 mL). The organic phase was dried over anhydrous sodium sulfate and filtered, and the filtrate was concentrated under reduced pressure. The crude product was purified by preparative thin-layer chromatography to give the product (70.0 mg, yield: 25.6%).

¹HNMR (300 MHz, DMSO-d6) δ (ppm): 10.10 (s, 1H), 8.41-8.38 (d, J=9 Hz, 1H), 7.87-7.75 (m, 2H), 7.46-7.41 (m, 2H), 7.26 (s, 0.29H), 7.17-7.09 (m, 3H), 7.07 (s, 0.47H), 6.88 (s, 0.25H), 4.44-4.41 (m, 1H), 3.10-3.08 (d, J=6 Hz, 1H), 2.74-2.70 (d, J=12 Hz, 1H), 2.22 (s, 3H), 2.00-1.90 (m, 3H), 1.78-1.44 (m, 3H).

Molecular formula: C₂₁H₂₂F₂N₄O Precise molecular weight: 384.18 LC-MS (m/z)=385.12[M+H]⁺.

### Example 26: Synthesis of Compound (R)-5-bromo-2-(6-methyl-4-((1-methylpiperidin-3-yl)amino)phthalazin-1-yl)phenol_(Compound 88)

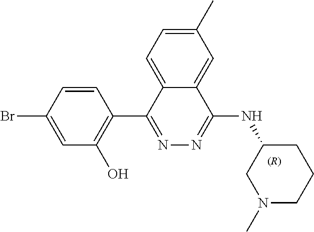

### Step 1: Synthesis of methyl 2-iodo-5-methylbenzoate

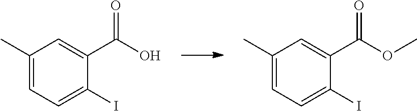

2-Iodo-5-methylbenzoic acid (20.0 g, 76.32 mmol, 1.0 eq.), iodomethane (16.25 g, 114.48 mmol, 1.5 eq.) and potassium carbonate (15.82 g, 114.48 mmol, 1.5 eq.) were dissolved in DMF (200 mL), and the mixture was reacted at room temperature for 0.5 h. After the reaction was completed as monitored by LC-MS, the reaction solution was poured into water (200 mL), followed by extraction with MTBE (200 mL×2). The organic phase was dried and concentrated to give the product (20 g, yield: 94.9%).

### Step 2: Synthesis of 4-bromo-2-methoxybenzoyl chloride

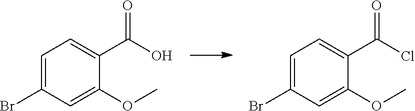

4-Bromo-2-methoxybenzoic acid (40.0 g, 173.12 mmol, 1.0 eq.) and DMF (0.5 mL) were dissolved in DCM (400 mL), followed by dropwise addition of oxalyl chloride (13.18 g, 103.87 mmol, 0.6 eq.). The mixture was reacted for 1 h. After the reaction was completed as monitored by TLC, the reaction solution was concentrated under reduced pressure to give the product (40 g, yield: 92.6%).

### Step 3: Synthesis of methyl 2-(4-bromo-2-methoxybenzoyl)-5-methylbenzoate

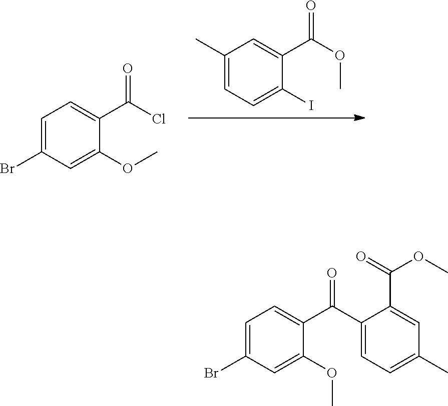

Methyl 2-iodo-5-methylbenzoate (20 g, 72.44 mmol, 1.0 eq.) was dissolved in THE (200 mL), and the mixture was cooled to −20° C., followed by addition of a solution of an isopropylmagnesium chloride lithium chloride complex in tetrahydrofuran (1.3 mol/L, 61.3 mL, 79.68 mmol, 1.1 eq.). The resulting mixture was stirred for 1 h, and a solution of 4-bromo-2-methoxybenzoyl chloride (18.07 g, 72.44 mmol, 1.0 eq.) in THE (200 mL) was added dropwise to the system. The mixture was reacted for 20 min. After the reaction was completed as monitored by LC-MS, the reaction solution was poured into water (200 mL), followed by extraction with MTBE (200 mL×2). The organic phase was dried and concentrated to give the product (20 g, yield: 76.0%).

### Step 4: Synthesis of 4-(4-bromo-2-methoxyphenyl)-7-methylphthalazin-1-ol

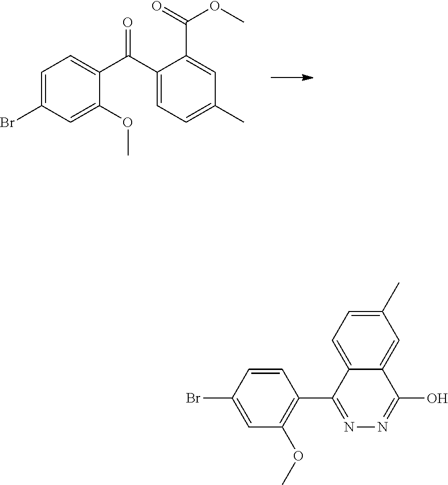 [SMILES: COC(=O)c1cc(C)ccc1C(=O)c1ccc(Br)cc1OC ; COc1cc(Br)ccc1-c1nnc(O)c2cc(C)ccc12]

Methyl 2-(4-bromo-2-methoxybenzoyl)-5-methylbenzoate (20.0 g, 55.06 mmol, 1.0 eq.) and 85% hydrazine hydrate (4.13 g, 82.59 mmol, 1.5 eq.) were dissolved in ethanol (100 mL), and the mixture was reacted at 80° C. for 1 h. After the reaction was completed as monitored by LC-MS, the reaction solution was cooled to room temperature and filtered, and the filter cake was dried under reduced pressure to give the product (11 g, yield: 57.8%).

### Step 5: Synthesis of 1-(4-bromo-2-methoxyphenyl)-4-chloro-6-methylphthalazine

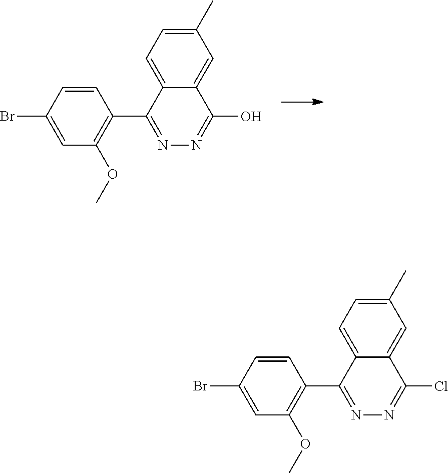 [SMILES: COc1cc(Br)ccc1-c1nnc(O)c2cc(C)ccc12 ; COc1cc(Br)ccc1-c1nnc(Cl)c2cc(C)ccc12]

4-(4-Bromo-2-methoxyphenyl)-7-methylphthalazin-1-ol (11.0 g, 31.86 mmol, 1.0 eq.) and phosphorus oxychloride (9.77 g, 63.72 mmol, 2.0 eq.) were dissolved in acetonitrile (110 mL), and the mixture was reacted at 90° C. for 1 h. After the reaction was completed as monitored by LC-MS, the reaction solution was concentrated under reduced pressure, and the crude product was completely dissolved in DCM (300 mL). Water (300 mL) was added, and the pH was adjusted to 9 with NaHCO₃, followed by liquid separation. The aqueous phase was extracted with DCM (100 mL), and the organic phases were combined, dried and concentrated to give the product (10 g, yield: 86.3%).

### Step 6: Synthesis of tert-butyl (R)-3-((4-(4-bromo-2-methoxyphenyl)-7-methylphthalazin-1-yl)amino)piperidine-1-carboxylate

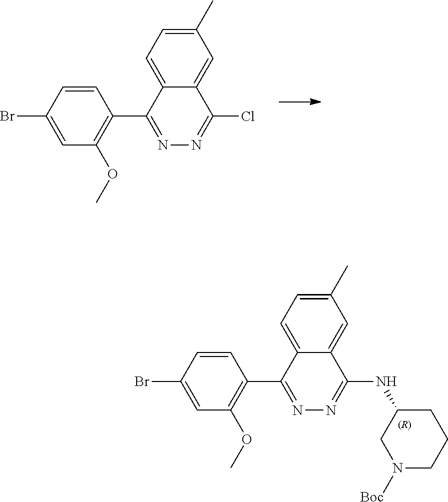

1-(4-Bromo-2-methoxyphenyl)-4-chloro-6-methylphthalazine (10 g, 27.50 mmol, 1.0 eq.), tert-butyl (R)-3-aminopiperidine-1-carboxylate (8.26 g, 41.25 mmol, 1.5 eq.) and DIPEA (10.66 g, 82.50 mmol, 3.0 eq.) were dissolved in DMAC (100 mL), and the mixture was reacted at 120° C. for 12 h. After the reaction was completed as detected by TLC, the reaction solution was poured into water (200 mL), followed by extraction with EA (200 mL×2). The organic phases were combined, washed with water (200 mL×2), dried and concentrated, and the crude product was purified by silica gel column chromatography (PE:EA=2:1) to give the product (11 g, yield: 75.8%).

### Step 7: Synthesis of (R)-5-bromo-2-(6-methyl-4-(piperidin-3-ylamino)phthalazin-1-yl) phenol

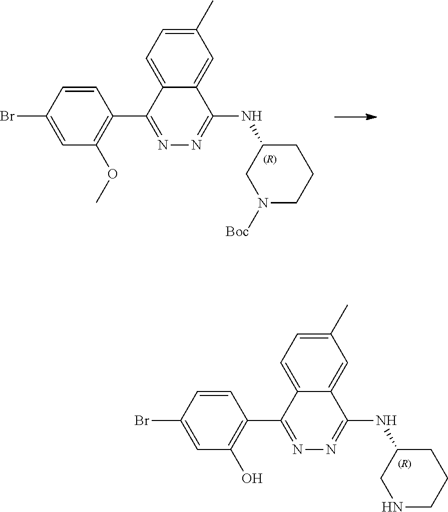

Tert-butyl (R)-3-((4-(4-bromo-2-methoxyphenyl)-7-methylphthalazin-1-yl)amino)piperidine-1-carboxylate (11 g, 20.85 mmol, 1.0 eq.) was dissolved in DCM (220 mL), followed by dropwise addition of boron tribromide (15.7 g, 62.55 mmol, 3.0 eq.). The mixture was reacted for 1 h. After the reaction was completed as detected by LC-MS, an appropriate amount of methanol was added to the system, and the reaction solution was concentrated under reduced pressure. The crude product was completely dissolved in water (50 mL), followed by liquid separation. The pH of the aqueous phase was adjusted to 9 with NaHCO₃, followed by extraction with a mixed solvent of dichloromethane and methanol (10:1, 100 mL×3). The organic phase was dried and concentrated to give the product (6.0 g, yield: 69.6%).

### Step 8: Synthesis of (R)-5-bromo-2-(6-methyl-4-((1-methylpiperidin-3-yl)amino)phthalazin-1-yl)phenol

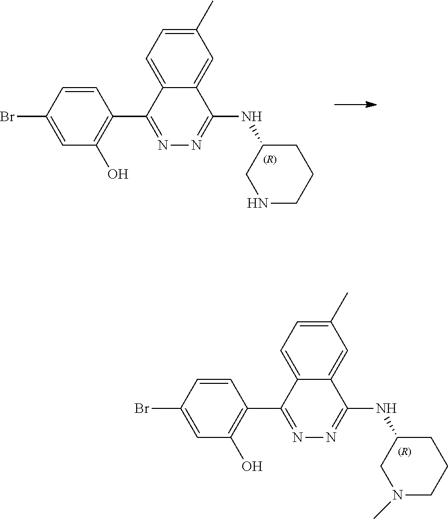

(R)-5-bromo-2-(6-methyl-4-(piperidin-3-ylamino)phthalazin-1-yl)phenol (6.0 g, 14.51 mmol, 1.0 eq.) was dissolved in methanol (60 mL), followed by addition of an aqueous formaldehyde solution (37%) (1.18 g, 14.51 mmol, 1.0 eq.). The mixture was stirred at room temperature for 5 min, followed by addition of sodium cyanoborohydride (911 mg, 14.51 mmol, 1.0 eq.). The mixture was reacted at room temperature for 5 min. After the reaction was completed as monitored by TLC, the reaction solution was concentrated under reduced pressure. The crude product was purified by silica gel column chromatography (DCM:MeOH=10:1) to give the product (4.5 g, yield: 72.5%).

¹HNMR (300 MHz, DMSO-d6) δ (ppm): 10.20-10.16 (d, 1H), 8.20 (s, 1H), 7.63-7.60 (d, 1H), 7.40-7.37 (d, 1H), 7.25-7.23 (d, 1H), 7.17-7.12 (m, 2H), 7.00-6.97 (d, 1H), 4.41 (s, 1H), 3.11-3.08 (d, 1H), 2.75-2.71 (d, 1H), 2.53 (s, 3H), 2.24 (s, 3H), 2.00-1.96 (d, 3H), 1.80-1.75 (d, 1H), 1.68-1.60 (m, 1H), 1.52-1.45 (m, 1H).

Molecular formula: C₂₁H₂₃BrN₄O Molecular weight: 426.11 LC-MS (m/z)=427.01/429.01[M+H]⁺.

### Example 27: Synthesis of Compound (R)-1-(4-cyclopropyl-2-hydroxyphenyl)-4-((1-methylpiperidin-3-yl)amino)phthalazine-6-carbonitrile (Compound 89)

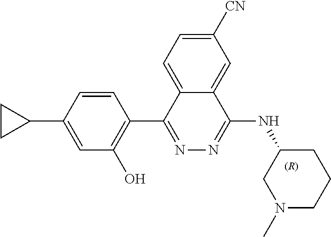

### Step 1: Synthesis of methyl 4-bromo-2-methoxybenzoate

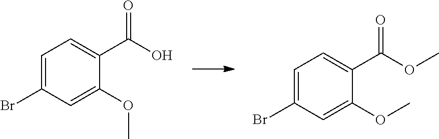

4-Bromo-2-methoxybenzoic acid (25.0 g, 108.20 mmol, 1.0 eq.), iodomethane (23.04 g, 162.30 mmol, 1.5 eq.) and potassium carbonate (22.40 g, 162.30 mmol, 1.5 eq.) were dissolved in DMF (250 mL), and the mixture was reacted at room temperature for 1 h. After the reaction was completed as monitored by TLC, the reaction solution was poured into water (500 mL), followed by extraction with MTBE (500 mL×2). The organic phase was washed with water (500 mL×2), dried and concentrated to give the product (26.5 g, yield: 100%).

### Step 2: Synthesis of methyl 4-cyclopropyl-2-methoxybenzoate

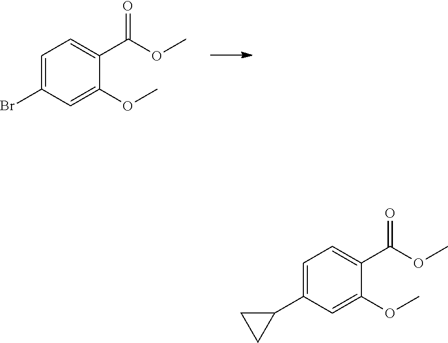

Methyl 4-bromo-2-methoxybenzoate (26.5 g, 108.20 mmol, 1.0 eq.), cyclopropylboronic acid (18.59 g, 216.40 mmol, 2.0 eq.), tricyclohexylphosphorus (15.20 g, 54.10 mmol, 0.5 eq.), palladium acetate (2.43 g, 10.82 mmol, 0.1 eq.) and potassium phosphate (45.94 g, 216.40 mmol, 2.0 eq.) were dissolved in toluene (520 mL) and water (100 mL), and the mixture was reacted at 100° C. for 23 h in nitrogen atmosphere. After the reaction was completed as detected by TLC, the reaction solution was concentrated under reduced pressure, the aqueous phase was extracted with EA (200 mL×3), and the organic phase was dried and concentrated under reduced pressure. The crude product was purified by silica gel column chromatography (PE:EA=20:1) to give the product (18 g, yield: 80.7%).

### Step 3: Synthesis of 4-cyclopropyl-2-methoxybenzoic acid

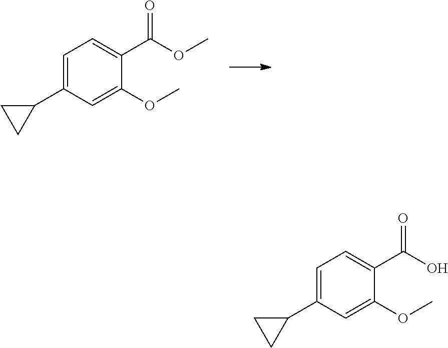

Methyl 4-cyclopropyl-2-methoxybenzoate (18 g, 87.27 mmol, 1.0 eq.) and lithium hydroxide monohydrate (7.32 g, 174.54 mmol, 2.0 eq.) were dissolved in MeOH (180 mL) and H₂O (90 mL), and the mixture was reacted at 40° C. for 3 h. After the reaction was completed as monitored by TLC, the reaction solution was concentrated under reduced pressure, and the crude product was completely dissolved in water (100 mL). The pH was adjusted to 3 with dilute hydrochloric acid, and the aqueous phase was extracted with DCM (200 mL×2), and the organic phase was washed with a 0.2 mol/L aqueous sodium hydroxide solution (300 mL). The aqueous phases were combined, adjusted to pH of 3 with dilute hydrochloric acid and extracted with DCM (200 mL×2). The organic phase was dried and concentrated to give the product (14 g, yield: 83.4%).

### Step 4: Synthesis of 4-cyclopropyl-2-methoxybenzoyl chloride

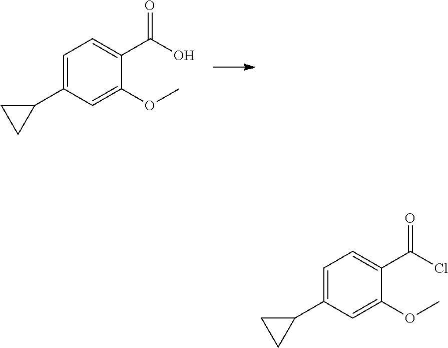

4-Cyclopropyl-2-methoxybenzoic acid (14 g, 72.83 mmol, 1.0 eq.) and DMF (0.15 mL) were dissolved in DCM (280 mL), followed by dropwise addition of oxalyl chloride (5.55 g, 43.70 mmol, 0.6 eq.). The mixture was reacted for 1 h, and the reaction solution was concentrated under reduced pressure to give the product (15.34 g, yield: 100%).

### Step 5: Synthesis of methyl 5-bromo-2-(4-cyclopropyl-2-methoxybenzoyl)benzoate

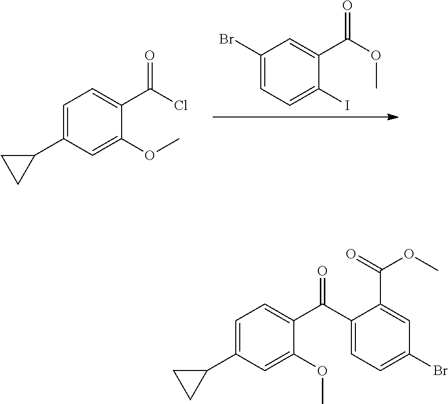 [SMILES: COc1cc(C2CC2)ccc1C(=O)Cl ; COC(=O)c1cc(Br)ccc1I ; COC(=O)c1cc(Br)ccc1C(=O)c1ccc(C2CC2)cc1OC]

Methyl 5-bromo-2-iodobenzoate (20 g, 58.66 mmol, 1.0 eq.) was dissolved in THE (200 mL), and the mixture was cooled to −30° C., followed by addition of a solution of an isopropylmagnesium chloride lithium chloride complex in tetrahydrofuran (1.3 mol/L, 49.6 mL, 64.53 mmol, 1.1 eq.). The resulting mixture was stirred for 0.5 h, and a solution of 4-cyclopropyl-2-methoxybenzoyl chloride (13.6 g, 64.53 mmol, 1.1 eq.) in THE (100 mL) was added dropwise to the system. The mixture was reacted for 20 min. After the reaction was completed as monitored by TLC, the reaction solution was poured into water (200 mL), followed by extraction with EA (200 mL×2). The organic phase was dried and concentrated to give the product (22.83 g, yield: 100%).

### Step 6: Synthesis of 7-bromo-4-(4-cyclopropyl-2-methoxyphenyl)phthalazin-1-ol

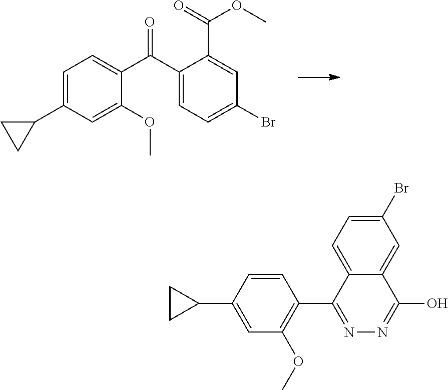 [SMILES: COC(=O)c1cc(Br)ccc1C(=O)c1ccc(C2CC2)cc1OC ; COc1cc(C2CC2)ccc1-c1nnc(O)c2cc(Br)ccc12]

Methyl 5-bromo-2-(4-cyclopropyl-2-methoxybenzoyl)benzoate (22.83 g, 58.66 mmol, 1.0 eq.) and 85% hydrazine hydrate (4.99 g, 117.32 mmol, 2.0 eq.) were dissolved in ethanol (150 mL), and the mixture was reacted at 80° C. for 0.5 h. After the reaction was completed as monitored by TLC, the reaction solution was cooled to room temperature and filtered, and the filter cake was dried to give the product (9 g, yield: 41.5%).

### Step 7: Synthesis of 6-bromo-4-chloro-1-(4-cyclopropyl-2-methoxyphenyl)phthalazine

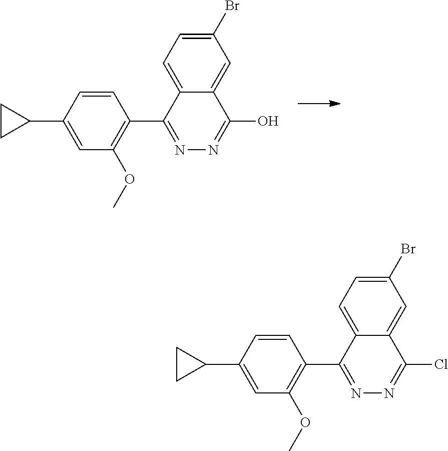 [SMILES: COc1cc(C2CC2)ccc1-c1nnc(O)c2cc(Br)ccc12 ; COc1cc(C2CC2)ccc1-c1nnc(Cl)c2cc(Br)ccc12]

7-Bromo-4-(4-cyclopropyl-2-methoxyphenyl)phthalazin-1-ol (9.0 g, 24.24 mmol, 1.0 eq.) and phosphorus oxychloride (7.43 g, 48.48 mmol, 2.0 eq.) were dissolved in acetonitrile (90 mL), and the mixture was reacted at 90° C. for 3 h. After the reaction was completed as monitored by TLC, the reaction solution was concentrated under reduced pressure, and the crude product was dissolved in EA (300 mL). The pH was adjusted to 9 with an aqueous NaHCO₃ solution, followed by liquid separation. The aqueous phase was extracted with EA (50 mL), and the organic phases were combined, dried and concentrated. The crude product was slurried with PE:EA (20:1, 100 mL) and filtered under vacuum, and the filter cake was dried to give the product (8.0 g, yield: 84.7%).

### Step 8: Synthesis of 2-(6-bromo-4-chlorophthalazin-1-yl)-5-cyclopropylphenol

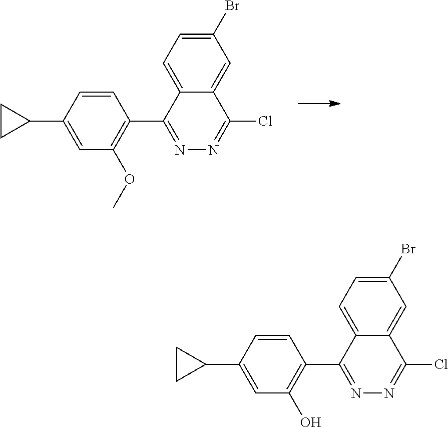 [SMILES: COc1cc(C2CC2)ccc1-c1nnc(Cl)c2cc(Br)ccc12 ; Oc1cc(C2CC2)ccc1-c1nnc(Cl)c2cc(Br)ccc12]

6-Bromo-4-chloro-1-(4-cyclopropyl-2-methoxyphenyl)phthalazine (8.0 g, 20.53 mmol, 1.0 eq.) was dissolved in DCM (160 mL), followed by dropwise addition of a 1 mol/L solution of boron tribromide in dichloromethane (30.8 mL, 30.80 mmol, 1.5 eq.). The mixture was reacted for 1 h. After the reaction was completed as detected by TLC, the reaction solution was poured into saturated NaHCO₃ (200 mL), and the mixture was stirred for 10 min, followed by liquid separation. The aqueous phase was extracted with DCM (100 mL), and the organic phases were combined, dried and concentrated to give the product (6.0 g, yield: 77.8%).

### Step 9: Synthesis of tert-butyl (R)-3-((7-bromo-4-(4-cyclopropyl-2-hydroxyphenyl)phthalazin-1-yl)amino)piperidine-1-carboxylate

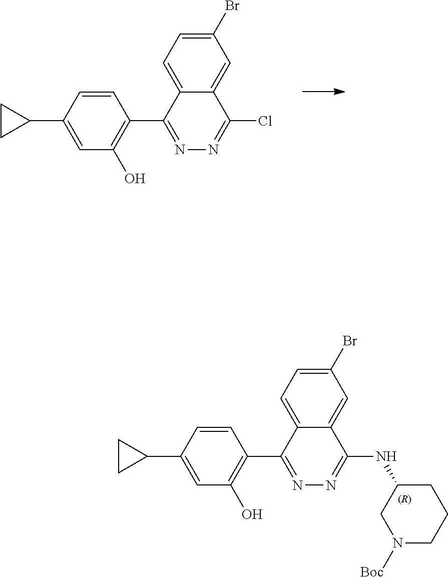

2-(6-Bromo-4-chlorophthalazin-1-yl)-5-cyclopropylphenol (6.0 g, 15.97 mmol, 1.0 eq.) and tert-butyl (R)-3-aminopiperidine-1-carboxylate (6.40 g, 91.94 mmol, 2.0 eq.) were dissolved in toluene (500 mL), and the mixture was reacted at 100° C. for 18 h. After the reaction was completed as detected by TLC, the reaction solution was concentrated under reduced pressure, and the crude product was purified by silica gel column chromatography (PE:EA=2:1) to give the product (2.3 g, yield: 26.7%).

### Step 10: Synthesis of (R)-2-(6-bromo-4-(piperidin-3-ylamino)phthalazin-1-yl)-5-cyclopropylphenol

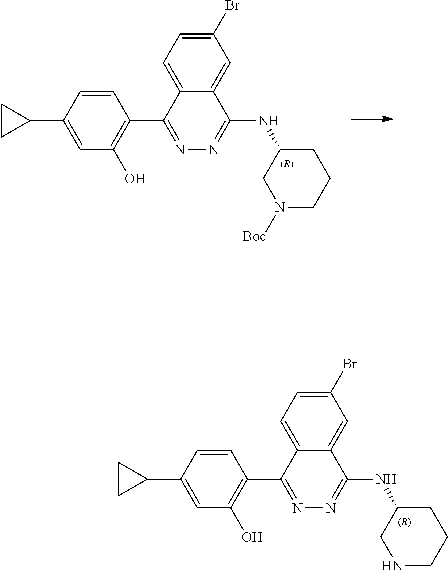

Tert-butyl (R)-3-((7-bromo-4-(4-cyclopropyl-2-hydroxyphenyl)phthalazin-1-yl)amino)piperidine-1-carboxylate (2.0 g, 3.71 mmol, 1.0 eq.) was dissolved in EA (20 mL), followed by dropwise addition of a solution of hydrogen chloride in 1,4-dioxane (4 mol/L, 20 mL). The mixture was reacted at room temperature for 2 h. After the reaction was completed as monitored by LC-MS, the reaction solution was concentrated under reduced pressure, and the crude product was completely dissolved in water (20 mL). The aqueous phase was extracted with EA (20 mL), and the pH of the aqueous phase was adjusted to 9 with NaHCO₃, followed by extraction with DCM (20 mL×3). The organic phase was dried and concentrated to give the product (1.3 g, yield: 79.7%).

### Step 11: Synthesis of (R)-2-(6-bromo-4-((1-methylpiperidin-3-yl)amino)phthalazin-1-yl)-5-cyclopropylphenol

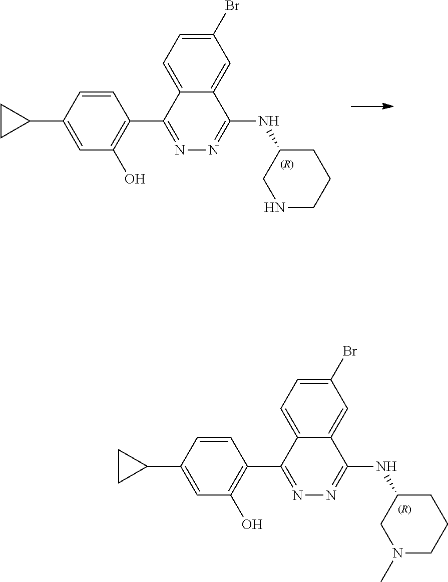

(R)-2-(6-bromo-4-(piperidin-3-ylamino)phthalazin-1-yl)-5-cyclopropylphenol (1.3 g, 2.95 mmol, 1.0 eq.) was dissolved in methanol (13 mL), followed by addition of an aqueous formaldehyde solution (37%) (240 mg, 2.95 mmol, 1.0 eq.). The mixture was stirred at room temperature for 10 min, followed by addition of sodium cyanoborohydride (186 mg, 2.95 mmol, 1.0 eq.). The mixture was reacted at room temperature for 10 min. After the reaction was completed as monitored by TLC, the reaction solution was concentrated under reduced pressure, and the crude product was dispersed with water (20 mL) and extracted with DCM (20 mL×2). The organic phase was dried and concentrated, and the crude product was purified by silica gel column chromatography (DCM:MeOH=50:1) to give the product (1.0 g, yield: 74.6%).

### Step 12: Synthesis of (R)-1-(4-cyclopropyl-2-hydroxyphenyl)-4-((1-methylpiperidin-3-yl)amino)phthalazine-6-carbonitrile

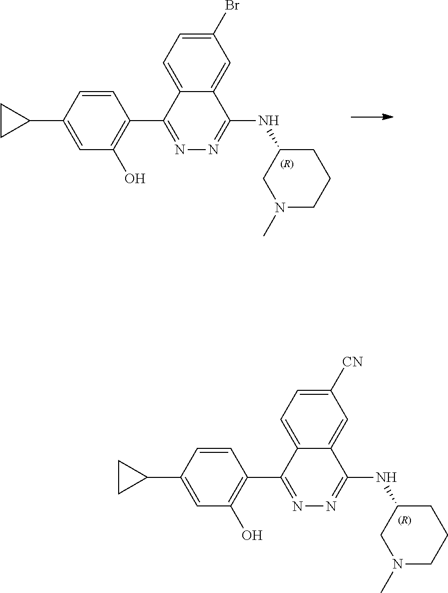

(R)-2-(6-bromo-4-((1-methylpiperidin-3-yl)amino)phthalazin-1-yl)-5-cyclopropylphenol (500 mg, 1.10 mmol, 1.0 eq.), zinc cyanide (130 mg, 1.10 mmol, 1.0 eq.) and tetrakis(triphenylphosphine)palladium (127 mg, 0.11 mmol, 0.1 eq.) were added to DMAC (10 mL), and the mixture was reacted at 120° C. for 3 h in nitrogen atmosphere. After the reaction was completed as monitored by LC-MS, the reaction solution was poured into water (20 mL), and the aqueous phase was extracted with EA (20 mL×2). The organic phases were combined, washed with water (20 mL×3), dried and concentrated, and the crude product was purified by preparative thin-layer chromatography (MeOH:DCM=1:10) to give the product (150 mg, yield: 34.2%).

¹HNMR (400 MHz, DMSO-d6) δ (ppm): 9.6 (s, 1H), 9.08 (s, 1H), 8.13-8.11 (m, 1H), 7.61-7.59 (d, 1H), 7.53 (s, 1H), 7.18-7.16 (d, 1H), 6.71-6.68 (t, 2H), 4.54 (s, 1H), 2.96 (s, 1H), 2.47 (s, 4H), 2.01-1.90 (m, 3H), 1.72-1.59 (t, 3H), 1.23 (s, 1H), 1.01-0.98 (m, 2H), 0.71-0.69 (m, 2H).

Molecular formula: C₂₄H₂₅N₅O Precise molecular weight: 399.21 LC-MS (m/z)=400.15[M+H]⁺.

### Example 28: Synthesis of Compound (R)-5-cyclopropyl-2-(6-methyl-4-((1-methylpiperidin-3-yl)amino)phthalazin-1-yl)phenol (Compound 90)

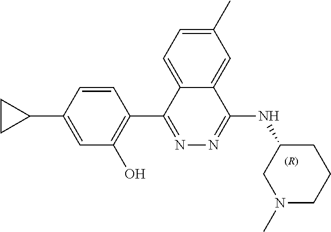

### Step 1: Synthesis of (R)-5-cyclopropyl-2-(6-methyl-4-((1-methylpiperidin-3-yl)amino)phthalazin-1-yl)phenol

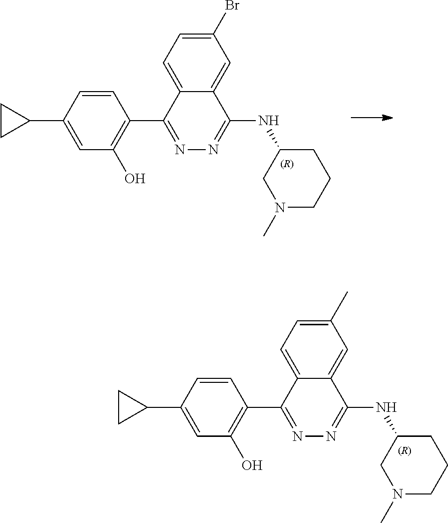

(R)-2-(6-bromo-4-((1-methylpiperidin-3-yl)amino)phthalazin-1-yl)-5-cyclopropylphenol (500 mg, 1.10 mmol, 1.0 eq.), 50% trimethylboroxine (1.10 g, 4.40 mmol, 4.0 eq.), cesium carbonate (717 mg, 2.20 mmol, 2.0 eq.) and [1,1′-bis(diphenylphosphino)ferrocene]palladium dichloride (80 mg, 0.11 mmol, 0.1 eq.) were dissolved in H₂O (2 mL) and 1,4-dioxane (10 mL), and the mixture was reacted at 100° C. for 18 h in nitrogen atmosphere. After the reaction was completed as monitored by LC-MS, the reaction solution was poured into water (20 mL), followed by extraction with EA (20 mL×2). The organic phase was dried and concentrated, and the crude product was purified by preparative thin-layer chromatography (MeOH:DCM=1:10) to give the product (110 mg, yield: 25.7%).

¹HNMR (400 MHz, DMSO-d6) δ (ppm): 9.72 (s, 1H), 8.27 (s, 1H), 7.65-7.62 (m, 1H), 7.48-7.46 (d, 1H), 7.27 (s, 1H), 7.17-7.15 (d, 1H), 6.70-6.66 (m, 2H), 4.62 (s, 1H), 3.48 (s, 1H), 3.14 (s, 1H), 2.65 (s, 3H), 2.54 (s, 3H), 1.99-1.91 (m, 3H), 1.83-1.77 (m, 3H), 1.01-0.97 (m, 2H), 0.72-0.69 (m, 2H).

Molecular formula: C₂₄H₂₈N₄O₁ Precise molecular weight: 388.23 LC-MS (m/z)=389.16[M+H]⁺.

### Example 29: Synthesis of Compound (R)-2-(4-((1-methylpiperidin-3-yl)amino)phthalazin-1-yl)-5-(trifluoromethyl)phenol (Compound 93)

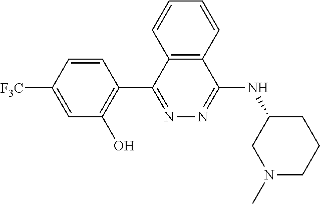 [SMILES: CN1CCC[C@@H](Nc2nnc(-c3ccc(C(F)(F)F)cc3O)c3ccccc23)C1]

### Step 1: Synthesis of 1-bromo-2-(ethoxymethoxy)-4-(trifluoromethyl)benzene

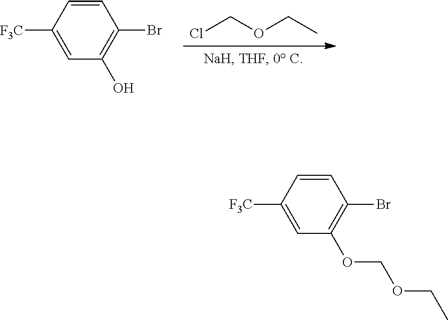

2-Bromo-5-trifluoromethylphenol (5.00 g, 20.7 mmol, 1.0 eq.) was dissolved in THF (50 mL), and the mixture was cooled to 0° C., followed by portionwise addition of NaH (60%, 2.94 g, 31.1 mmol, 1.5 eq.). The resulting mixture was stirred for 20 min, and chloromethyl ethyl ether (1.24 g, 31.1 mmol, 1.5 eq.) was added. After 4 h of reaction, saturated aqueous NH₄Cl solution was added to quench the reaction, followed by extraction with EA (50 mL×2). The organic phase was dried over anhydrous sodium sulfate and filtered, and the filtrate was concentrated under reduced pressure to give the product (6.08 g, yield: 98.0%).

### Step 2: Synthesis of (2-(ethoxymethoxy)-4-(trifluoromethyl)phenyl)boronic acid

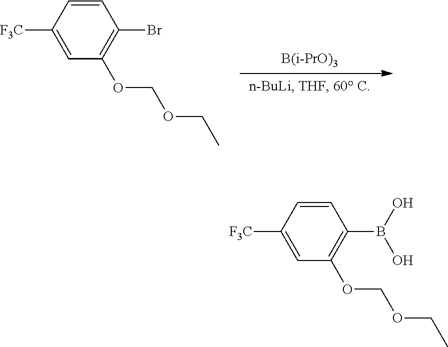 [SMILES: CCOCOc1cc(C(F)(F)F)ccc1Br ; "n-BuLi, THF, 60° C." ; CCOCOc1cc(C(F)(F)F)ccc1B(O)O ; "B(i-PrO)3"]

1-Bromo-2-(ethoxymethoxy)-4-(trifluoromethyl)benzene (3.00 g, 10.0 mmol, 1.0 eq.) and triisopropyl borate (2.82 g, 15.0 mmol, 1.5 eq.) were dissolved in THF (30 mL), and the mixture was cooled to −60° C. in nitrogen atmosphere, followed by dropwise addition of n-butyllithium (1.6 mol/L THF solution, 9.4 mL, 15.0 mmol, 1.5 eq.). The mixture was reacted for 3 h. a saturated aqueous NH₄Cl solution was added to quench the reaction, followed by extraction with EA (50 mL). The organic phase was dried over anhydrous sodium sulfate and filtered, and the filtrate was concentrated under reduced pressure. The crude product was slurried with petroleum ether to give the product (2.20 g, yield: 83.1%).

### Step 3: Synthesis of (R)-4-(2-(ethoxymethoxy)-4-(trifluoromethyl)phenyl)-N-(1-methylpiperidin-3-yl)phthalazin-1-amine

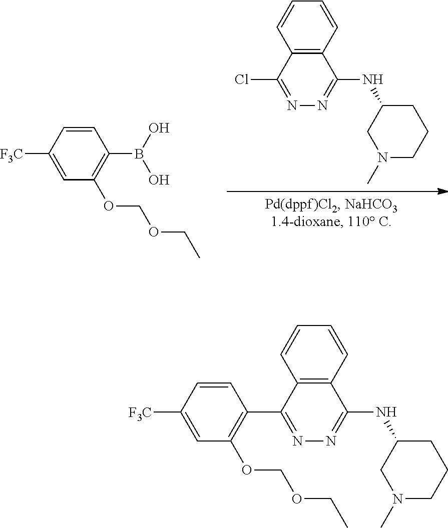 [SMILES: CCOCOc1cc(C(F)(F)F)ccc1B(O)O ; CN1CCC[C@@H](Nc2nnc(Cl)c3ccccc23)C1 ; "Pd(dppf)Cl2, NaHCO31.4-dioxane, 110° C." ; CCOCOc1cc(C(F)(F)F)ccc1-c1nnc(N[C@@H]2CCCN(C)C2)c2ccccc12]

(2-(Ethoxymethoxy)-4-(trifluoromethyl)phenyl)boronic acid (229 mg, 0.867 mmol, 1.2 eq.), (R)-4-chloro-N-(1-methylpiperidin-3-yl)phthalazin-1-amine (200 mg, 0.723 mmol, 1.0 eq.), Pd(dppf)Cl₂ (26.5 mg, 0.0362 mmol, 0.05 eq.) and NaHCO₃ (122 mg, 1.45 mmol, 2.0 eq.) were added successively to 1,4-dioxane (4 mL), followed by addition of H₂O (2 mL). The mixture was heated to 110° C. and reacted for 3 h in nitrogen atmosphere. The reaction solution was cooled to room temperature, and water (30 mL) was added to quench the reaction, followed by extraction with EA (30 mL×2). The organic phase was dried over anhydrous sodium sulfate and filtered, and the filtrate was concentrated under reduced pressure. The crude product was purified by silica gel column chromatography (DCM:MeOH=50:1 to 20:1) to give the product (245 mg, yield: 73.6%).

### Step 4: Synthesis of (R)-2-(4-((1-methylpiperidin-3-yl)amino)phthalazin-1-yl)-5-(trifluoromethyl)phenol

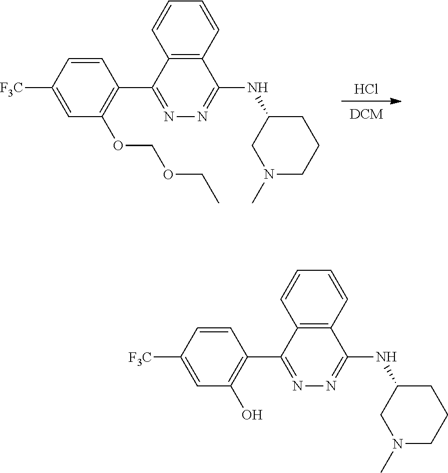

(R)-4-(2-(ethoxymethoxy)-4-(trifluoromethyl)phenyl)-N-(1-methylpiperidin-3-yl)phthalazin-1-amine (245 mg, 0.532 mmol, 1.0 eq.) was dissolved in DCM (4 mL), followed by dropwise addition of a solution of hydrogen chloride in 1,4-dioxane (4 mol/L, 1.3 mL, 5.32 mmol, 10.0 eq.). The mixture was stirred at room temperature for 1 h. Then water (20 mL) was added to quench the reaction, followed by washing with DCM (20 mL×2). The pH of the aqueous phase was adjusted to 8 with a saturated aqueous NaHCO₃ solution, followed by extraction with DCM (20 mL×2). The organic phase was dried over anhydrous sodium sulfate and filtered, and the filtrate was concentrated under reduced pressure to give the product (166 mg, yield: 77.5%).

¹HNMR (400 MHz, DMSO-d6) δ (ppm): 10.51 (s, 1H), 8.55 (s, 1H), 7.91-7.88 (m, 1H), 7.83-7.80 (m, 1H), 7.59 (s, 1H), 7.51 (d, J=7.8 Hz, 1H), 7.46 (d, J=8.0 Hz, 1H), 7.35 (s, 1H), 7.31 (d, J=7.9 Hz, 1H), 4.73 (s, 1H), 3.56 (s, 2H), 3.22 (s, 2H), 2.73 (s, 3H), 1.99 (s, 2H), 1.83 (d, J=9.2 Hz, 2H).

Molecular formula: C₂₁H₂₁F₃N₄O Precise molecular weight: 402.17 LC-MS (m/z)=403.11[M+H]⁺.

### Example 30: Synthesis of Compound (R)-5-methyl-2-(4-((1-methylpiperidin-3-yl)amino)phthalazin-1-yl)pyridin-3-ol (Compound 108)

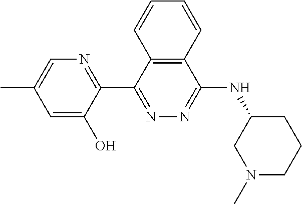 [SMILES: Cc1cnc(-c2nnc(N[C@@H]3CCCN(C)C3)c3ccccc23)c(O)c1]

### Step 1: Synthesis of 5-methyl-3-hydroxypyridine

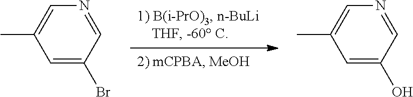

3-Bromo-5-methylpyridine (20.0 g, 0.116 mol, 1.0 eq.) and triisopropyl borate (26.1 g, 0.139 mol, 1.2 eq.) were dissolved in THF (200 mL), and the mixture was cooled to −60° C. in nitrogen atmosphere, followed by dropwise addition of n-butyllithium (2.5 mol/L THF solution, 55.6 mL, 0.139 mol, 1.2 eq.). The mixture was reacted for 2 h. Dilute hydrochloric acid was added to quench the reaction, and the pH was adjusted to 3, followed by liquid separation. The aqueous phase was extracted with EA (100 mL×3), the organic phases were combined, dried over anhydrous sodium sulfate and filtered, and the filtrate was concentrated under reduced pressure. The resulting crude product was dissolved in MeOH (100 mL), followed by addition of meta-chloroperoxybenzoic acid (16.0 g, 0.0928 mol, 0.8 eq.). After the mixture was stirred at room temperature for 1 h, a saturated aqueous sodium thiosulfate solution was added to quench the reaction, followed by extraction with DCM (100 mL×3). The organic phase was dried over anhydrous sodium sulfate and concentrated, and the crude product was purified by silica gel column chromatography (DCM:MeOH=100:1 to 20:1) to give the product (10.8 g, yield: 85.1%).

### Step 2: Synthesis of 2-iodo-5-methyl-3-hydroxypyridine

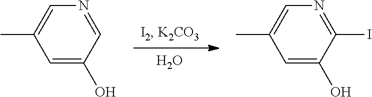

5-Methyl-3-hydroxypyridine (10.0 g, 0.0916 mol, 1.0 eq.) and KOH (10.3 g, 0.183 mol, 2.0 eq.) were added to water (500 mL), followed by addition of iodine (23.2 g, 0.0916 mol, 1.0 eq.). The mixture was reacted at room temperature for 16 h. The pH was adjusted to 7 with a saturated aqueous NH₄Cl solution, followed by extraction with EA (100 mL×3). The organic phase was dried over anhydrous sodium sulfate and filtered, and the filtrate was concentrated under reduced pressure. The crude product was purified by silica gel column chromatography (DCM:MeOH=100:1 to 50:1) to give the product (11.0 g, yield: 51.1%).

### Step 3: Synthesis of 2-iodo-3-methoxy-5-methylpyridine

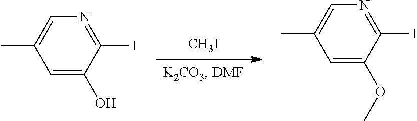

2-Iodo-5-methyl-3-hydroxypyridine (5.0 g, 21.3 mmol, 1.0 eq.) was dissolved in DMF (50 mL), followed by addition of iodomethane (4.53 g, 31.9 mmol, 1.5 eq.) and K₂CO₃ (5.89 g, 42.6 mmol, 2.0 eq.). The mixture was reacted at room temperature for 2 h. Water (250 mL) was added to quench the reaction, followed by extraction with EA (100 mL). The organic phase was washed with water (50 mL×3), dried over anhydrous sodium sulfate and filtered, and the filtrate was concentrated under reduced pressure. The crude product was purified by silica gel column chromatography (DCM:MeOH=100:1 to 50:1) to give the product (4.20 g, yield: 79.3%).

### Step 4: Synthesis of 2-(3-methoxy-5-methylpyridinoyl)benzoic acid

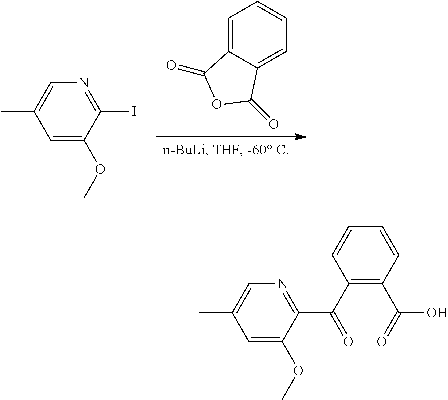

2-Iodo-3-methoxy-5-methylpyridine (4.00 g, 16.1 mmol, 1.0 eq.) was dissolved in THE (40 mL), and the mixture was cooled to −60° C. in nitrogen atmosphere, followed by dropwise addition of n-butyllithium (2.5 mol/L THE solution, 6.44 mL, 16.1 mmol, 1.0 eq.). The resulting mixture was stirred for 30 min. Phthalic anhydride (2.38 g, 16.1 mmol, 1.0 eq.) was dissolved in THE (40 mL), and the mixture was cooled to −60° C. and then added dropwise to the above reaction solution. After the completion of dropwise addition, the resulting mixture was reacted for 1 h. The pH was adjusted to 6 with dilute hydrochloric acid, followed by extraction with EA. The organic phase was dried over anhydrous sodium sulfate and filtered, and the filtrate was concentrated under reduced pressure to give the product (3.20 g, crude product).

### Step 5: Synthesis of methyl 2-(3-methoxy-5-methylpyridinoyl)benzoate

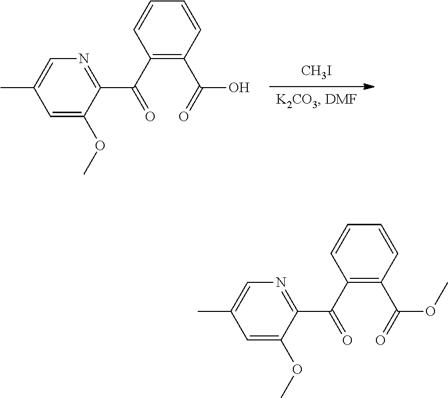

2-(3-Methoxy-5-methylpyridinoyl)benzoic acid (3.2 g, 11.8 mmol, 1.0 eq.) was dissolved in DMF (50 mL), followed by addition of K₂CO₃ (3.26 g, 23.6 mmol, 2.0 eq.) and iodomethane (2.51 g, 17.7 mmol, 1.5 eq.). The mixture was reacted at room temperature for 1 h. Water (200 mL) was added to quench the reaction, followed by extraction with EA (100 mL). The organic phase was washed with water (50 mL×3), dried over anhydrous sodium sulfate and filtered, and the filtrate was concentrated under reduced pressure. The crude product was purified by silica gel column chromatography (DCM:MeOH=100:1 to 50:1) to give the product (1.30 g, two-step yield: 28.4%).

### Step 6: Synthesis of 4-(3-methoxy-5-methylpyridin-2-yl)phthalazin-1-ol

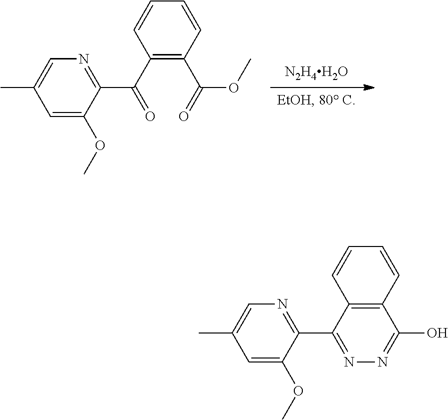

Methyl 2-(3-methoxy-5-methylpyridinoyl)benzoate (1.20 g, 4.21 mmol, 1.0 eq.) was dissolved in EtOH (12 mL), followed by addition of hydrazine hydrate (85%, 496 mg, 8.42 mmol, 2.0 eq.). The mixture was heated to 80° C. and reacted for 1 h. The reaction solution was cooled to room temperature and concentrated, and the crude product was purified by silica gel column chromatography (DCM:MeOH=100:1 to 20:1) to give the product (920 mg, yield: 81.8%).

### Step 7: Synthesis of 1-chloro-4-(3-methoxy-5-methylpyridin-2-yl)phthalazine

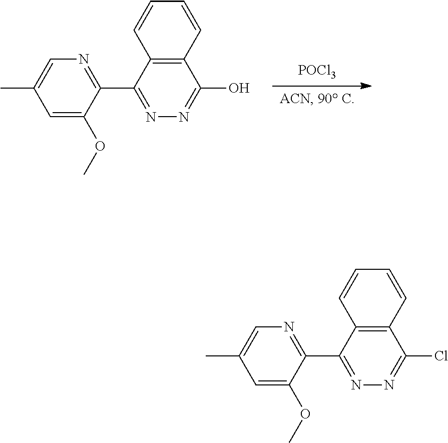 [SMILES: COc1cc(C)cnc1-c1nnc(O)c2ccccc12 ; "POCl3" ; "ACN, 90° C." ; COc1cc(C)cnc1-c1nnc(Cl)c2ccccc12]

4-(3-Methoxy-5-methylpyridin-2-yl)phthalazin-1-ol (900 mg, 3.37 mmol, 1.0 eq.) was added to ACN (20 mL), followed by addition of POCl₃ (5.17 g, 33.7 mmol, 10.0 eq.). The mixture was heated to 90° C. and reacted for 4 h. The reaction solution was cooled to room temperature, water was added to quench the reaction, and the pH was adjusted to 8 with a saturated aqueous NaHCO₃ solution, followed by extraction with EA (50 mL×3). The organic phase was dried over anhydrous sodium sulfate and filtered, and the filtrate was concentrated under reduced pressure. The crude product was purified by silica gel column chromatography (DCM:MeOH=100:1 to 50:1) to give the product (610 mg, yield: 63.4%).

### Step 8: Synthesis of tert-butyl (R)-3-((4-(3-methoxy-5-methylpyridin-2-yl)phthalazin-1-yl)amino)piperidine-1-carboxylate

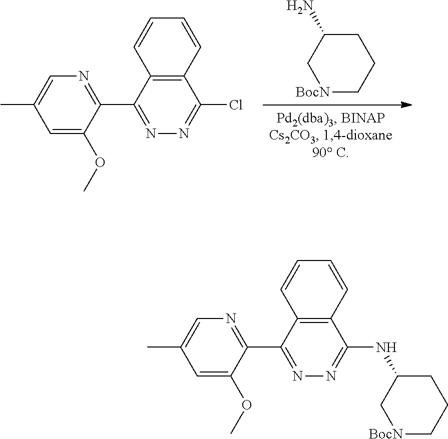

1-Chloro-4-(3-methoxy-5-methylpyridin-2-yl)phthalazine (600 mg, 2.10 mmol, 1.0 eq.), tert-butyl (R)-3-aminopiperidine-1-carboxylate (631 mg, 3.15 mmol, 1.5 eq.), Pd₂(dba)₃ (385 mg, 0.420 mmol, 0.2 eq.), BINAP (523 mg, 0.840 mmol, 0.4 eq.) and Cs₂CO₃ (1.37 g, 4.20 mmol, 2.0 eq.) were added successively to 1,4-dioxane (20 mL), and the mixture was heated to 90° C. and reacted for 6 h in nitrogen atmosphere. The reaction solution was cooled to room temperature, and water (50 mL) was added to quench the reaction, followed by extraction with EA (30 mL×2). The organic phase was dried over anhydrous sodium sulfate and filtered, and the filtrate was concentrated under reduced pressure. The crude product was purified by silica gel column chromatography (DCM:MeOH=100:1 to 20:1) to give the product (485 mg, yield: 51.4%).

### Step 9: Synthesis of (R)-5-methyl-2-(4-(piperidin-3-ylamino)phthalazin-1-yl)pyridin-3-ol

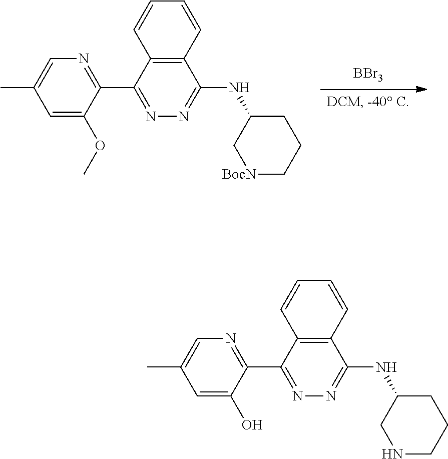 [SMILES: COc1cc(C)cnc1-c1nnc(N[C@@H]2CCCN(C(=O)OC(C)(C)C)C2)c2ccccc12 ; "BBr3" ; "DCM, -40° C." ; Cc1cnc(-c2nnc(N[C@@H]3CCCNC3)c3ccccc23)c(O)c1]

Tert-butyl (R)-3-((4-(3-methoxy-5-methylpyridin-2-yl)phthalazin-1-yl)amino)piperidine-1-carboxylate (485 mg, 1.08 mmol, 1.0 eq.) was dissolved in DCM (15 mL), and the mixture was cooled to −40° C., followed by dropwise addition of BBr₃ (2.71 g, 10.8 mmol, 10.0 eq.). The mixture was naturally warmed to room temperature and reacted for 20 h. The reaction solution was concentrated, MeOH was added to quench the reaction, and a saturated aqueous NaHCO₃ solution was added to adjust the pH to 8, and DCM (30 mL×3) was added for extraction. The organic phase was dried over anhydrous sodium sulfate and filtered, and the filtrate was concentrated under reduced pressure. The crude product was purified by silica gel column chromatography (DCM:MeOH=100:1 to 10:1) to give the product (160 mg, yield: 44.2%).

### Step 10: Synthesis of (R)-5-methyl-2-(4-((1-methylpiperidin-3-yl)amino)phthalazin-1-yl)pyridin-3-ol

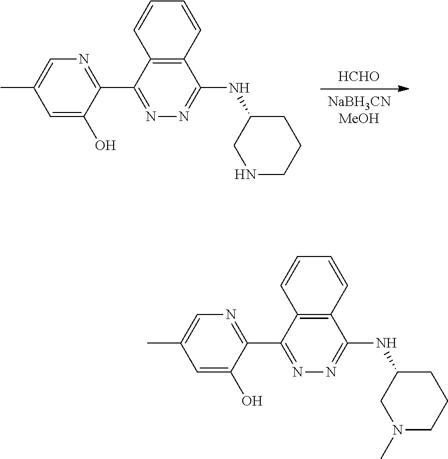 [SMILES: Cc1cnc(-c2nnc(N[C@@H]3CCCNC3)c3ccccc23)c(O)c1 ; "HCHO" ; "NaBH3CNMeOH" ; Cc1cnc(-c2nnc(N[C@@H]3CCCN(C)C3)c3ccccc23)c(O)c1]

(R)-5-methyl-2-(4-(piperidin-3-ylamino)phthalazin-1-yl)pyridin-3-ol (160 mg, 0.477 mmol, 1.0 eq.) was dissolved in MeOH (4 mL), followed by addition of an aqueous formaldehyde solution (37%, 116 mg, 1.43 mmol, 3.0 eq.). The mixture was stirred at room temperature for 30 min. NaBH₃CN (90.0 mg, 1.43 mmol, 3.0 eq.) was then added and the mixture was reacted for 1 h. A saturated aqueous NaHCO₃ solution was added to quench the reaction, followed by extraction with DCM (20 mL×3). The organic phase was dried over anhydrous sodium sulfate and filtered, and the filtrate was concentrated under reduced pressure. The crude product was purified by preparative thin-layer chromatography (DCM:MeOH=10:1) to give the product (70 mg, yield: 42.0%).

¹HNMR (400 MHz, DMSO-d6) δ (ppm): 14.29 (s, 1H), 9.63-9.61 (m, 1H), 8.62 (s, 1H), 8.11 (d, J=1.4 Hz, 1H), 7.99-7.94 (m, 2H), 7.82 (s, 1H), 7.25 (d, J=1.1 Hz, 1H), 4.64 (s, 1H), 3.39 (d, J=10.0 Hz, 2H), 3.05 (d, J=9.0 Hz, 1H), 2.57 (s, 4H), 2.34 (s, 3H), 2.01-1.92 (m, 2H), 1.79-1.74 (m, 2H).

Molecular formula: C₂₀H₂₃N₅O Precise molecular weight: 349.19 LC-MS (m/z)=350.14[M+H]⁺.

### Example 31: Synthesis of (R)-2-(6-fluoro-4-((1-methylpiperidin-3-yl)amino)phthalazin-1-yl)-5-methylphenol (Compound 8)

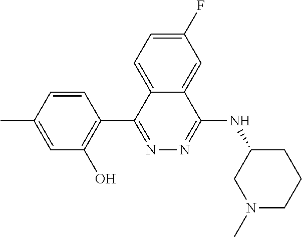 [SMILES: Cc1ccc(-c2nnc(N[C@@H]3CCCN(C)C3)c3cc(F)ccc23)c(O)c1]

### Step 1: Synthesis of methyl 5-fluoro-2-(2-methoxy-4-methylbenzoyl)benzoate

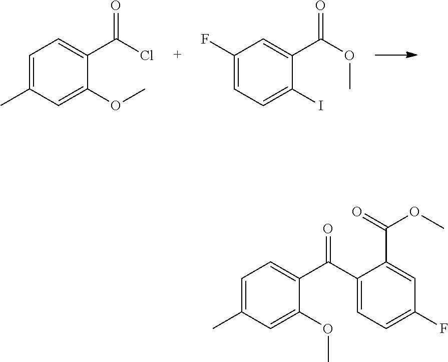 [SMILES: COc1cc(C)ccc1C(=O)Cl ; COC(=O)c1cc(F)ccc1I ; COC(=O)c1cc(F)ccc1C(=O)c1ccc(C)cc1OC]

Methyl 5-fluoro-2-iodobenzoate (5.0 g, 17.85 mmol, 1.0 eq.) was dissolved in THE (50 mL), and the mixture was cooled to −30° C., followed by dropwise and slow addition of a solution of an isopropylmagnesium chloride lithium chloride complex in tetrahydrofuran (1.3 mol/L, 16.5 mL, 21.42 mmol, 1.2 eq.). The resulting mixture was stirred for 30 min. The above reaction solution was added dropwise and slowly to a solution of 2-methoxy-4-methylbenzoyl chloride (3.30 g, 17.85 mmol, 1.0 eq.) in THE (30 mL) at −60° C. After the completion of dropwise addition, the mixture was naturally warmed to room temperature and reacted for 1 h. After the generation of the product as monitored by LC-MS, the reaction solution was poured into water (100 mL), followed by extraction with EA (50 mL×2). The organic phase was dried and concentrated to give the product (5.4 g, yield: 100%).

### Step 2: Synthesis of 7-fluoro-4-(2-methoxy-4-methylphenyl)phthalazin-1-ol

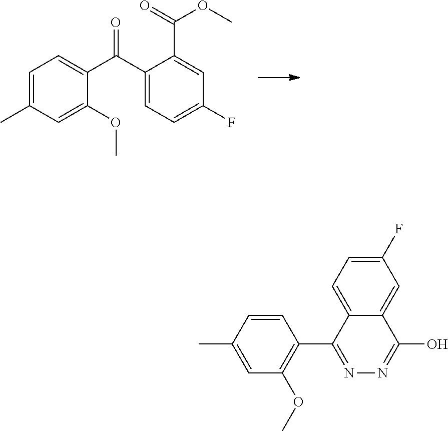 [SMILES: COC(=O)c1cc(F)ccc1C(=O)c1ccc(C)cc1OC ; COc1cc(C)ccc1-c1nnc(O)c2cc(F)ccc12]

Methyl 5-fluoro-2-(2-methoxy-4-methylbenzoyl)benzoate (5.4 g, 17.85 mmol, 1.0 eq.) and 85% hydrazine hydrate (1.05 g, 17.85 mmol, 1.0 eq.) were dissolved in ethanol (50 mL), and the mixture was reacted at room temperature for 12 h. After the generation of the product as monitored by LC-MS, the reaction solution was filtered to give the product (1.0 g, yield: 19.7%).

### Step 3: Synthesis of 4-chloro-6-fluoro-1-(2-methoxy-4-methylphenyl)phthalazine

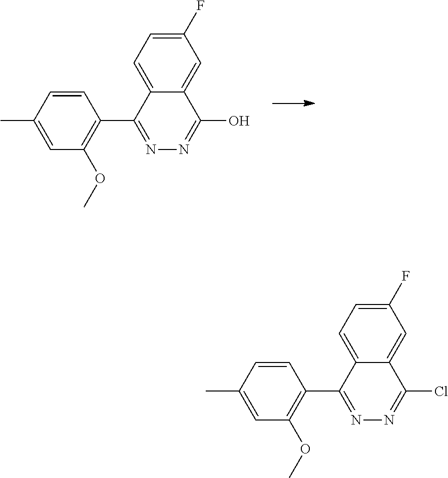 [SMILES: COc1cc(C)ccc1-c1nnc(O)c2cc(F)ccc12 ; COc1cc(C)ccc1-c1nnc(Cl)c2cc(F)ccc12]

7-Fluoro-4-(2-methoxy-4-methylphenyl)phthalazin-1-ol (1.0 g, 3.51 mmol, 1.0 eq.) and phosphorus oxychloride (1.07 g, 7.02 mmol, 2.0 eq.) were dissolved in acetonitrile (10 mL), and the mixture was reacted at 90° C. for 0.5 h. After the reaction was completed as monitored by LC-MS, the reaction solution was concentrated under reduced pressure, and the crude product was dissolved in EA (20 mL). The pH was adjusted to 9 with a saturated aqueous sodium bicarbonate solution, followed by liquid separation. The aqueous phase was extracted with EA (20 mL), and the organic phases were combined, dried and concentrated to give the product (0.9 g, yield: 84.9%).

### Step 4: Synthesis of tert-butyl (R)-3-((7-fluoro-4-(2-methoxy-4-methylphenyl)phthalazin-1-yl)amino)piperidine-1-carboxylate

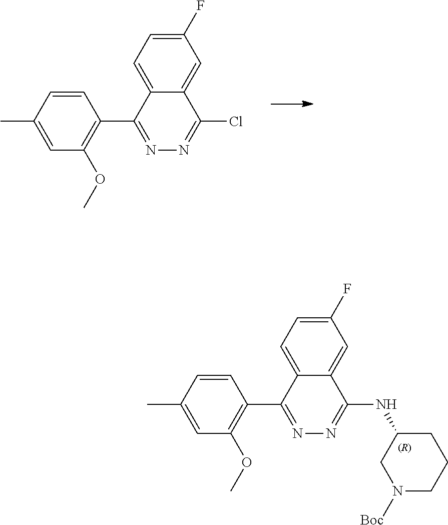

4-Chloro-6-fluoro-1-(2-methoxy-4-methylphenyl)phthalazine (0.9 g, 2.97 mmol, 1.0 eq.), tert-butyl (R)-3-aminopiperidine-1-carboxylate (894 mg, 4.46 mmol, 1.5 eq.), Pd₂(dba)₃ (274 mg, 0.30 mmol, 0.1 eq.), BINAP (367 mg, 0.59 mmol, 0.2 eq.) and cesium carbonate (1.94 g, 5.94 mmol, 2.0 eq.) were added to 1,4-dioxane (20 mL), and the mixture was reacted at 90° C. for 21 h in nitrogen atmosphere. After the reaction was completed as detected by LC-MS, the reaction solution was concentrated under reduced pressure, and the crude product was purified by silica gel column chromatography (MeOH:DCM=1:80) to give the product (0.8 g, yield: 57.9%).

### Step 5: Synthesis of (R)-2-(6-fluoro-4-(piperidin-3-ylamino)phthalazin-1-yl)-5-methylphenol

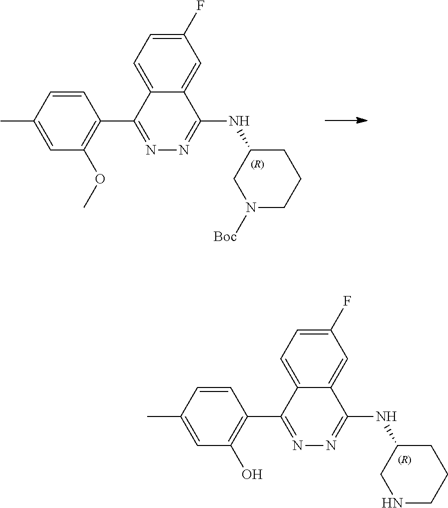

Tert-butyl (R)-3-((7-fluoro-4-(2-methoxy-4-methylphenyl)phthalazin-1-yl)amino)piperidine-1-carboxylate (0.8 g, 1.71 mmol, 1.0 eq.) was dissolved in DCM (10 mL), followed by dropwise addition of a 1 mol/L solution of boron tribromide in dichloromethane (5.2 mL, 5.13 mmol, 3.0 eq.). The mixture was reacted for 4 h. After the reaction was completed as detected by LC-MS, an appropriate amount of methanol was added to the flask to quench the reaction, and water (5 mL) was added, followed by liquid separation. The pH of the aqueous phase was adjusted to 9 with a saturated aqueous sodium bicarbonate solution, the aqueous phase was extracted with DCM (15 mL×3), and the organic phase was dried and concentrated to give the product (300 mg, yield: 49.8%).

### Step 6: Synthesis of (R)-2-(6-fluoro-4-((1-methylpiperidin-3-yl)amino)phthalazin-1-yl)-5-methylphenol

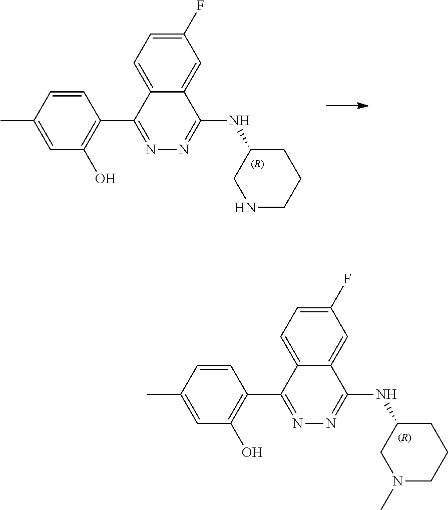

(R)-2-(6-fluoro-4-(piperidin-3-ylamino)phthalazin-1-yl)-5-methylphenol (300 mg, 0.85 mmol, 1.0 eq.) was dissolved in methanol (5 mL), followed by addition of an aqueous formaldehyde solution (37%) (69 mg, 0.85 mmol, 1.0 eq.). The mixture was stirred at room temperature for 10 min, followed by addition of sodium cyanoborohydride (53 mg, 0.85 mmol, 1.0 eq.). The mixture was reacted at room temperature for 10 min. After the reaction was completed as monitored by LC-MS, the reaction solution was concentrated under reduced pressure. The crude product was purified by silica gel column chromatography (DCM:MeOH=20:1) to give the product (160 mg, yield: 51.4%).

¹HNMR (400 MHz, DMSO-d6) δ (ppm): 9.59 (s, 1H), 8.28-8.25 (m, 1H), 7.69-7.64 (m, 1H), 7.61-7.57 (m, 1H), 7.18-7.16 (d, 1H), 7.03-7.01 (d, 1H), 6.81 (s, 1H), 6.79-6.77 (d, 1H), 4.41-4.39 (d, 1H), 3.14-3.12 (d, 1H), 2.78-2.75 (d, 1H), 2.33 (s, 3H), 2.26 (s, 1H), 2.02-1.99 (d, 3H), 1.79-1.76 (d, 1H), 1.64-1.58 (t, 1H), 1.49-1.41 (m, 1H).

Molecular formula: C₂₁H₂₃FN₄O Precise molecular weight: 366.19 LC-MS (Pos, m/z)=367.14[M+H]⁺.

### Example 32: Synthesis of (R)-3-hydroxy-4-(6-methyl-4-((1-methylpiperidin-3-yl)amino)phthalazin-1-yl)benzonitrile (Compound 55) and (R)-3-hydroxy-4-(7-methyl-4-((1-methylpiperidin-3-yl)amino)phthalazin-1-yl)benzonitrile (Compound 54)

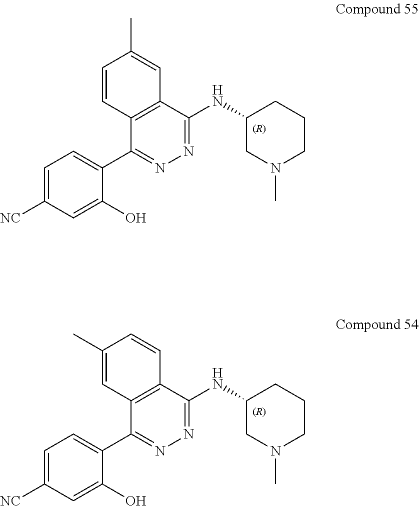

### Step 1: Synthesis of 6-methyl-2,3-dihydro-phthalazine-1,4-dione

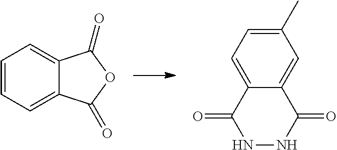

5-Methylisobenzofuran-1,3-dione (5 g, 30.83 mmol, 1.0 eq.) and 85% hydrazine hydrate (2.73 g, 46.25 mmol, 1.5 eq.) were added to 1,4-dioxane (100 mL) solution, and the mixture was reacted at 80° C. for 1 h. After the reaction was completed as monitored by LC-MS, the reaction solution was filtered under vacuum, and the filter cake was dried to give the product (5.43 g, yield: 100%).

### Step 2: Synthesis of 1,4-dichloro-6-methylphthalazine

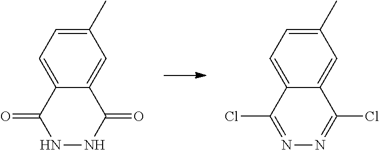

6-Methyl-2,3-dihydro-phthalazine-1,4-dione (5.43 g, 30.83 mmol, 1.0 eq.) and phosphorus oxychloride (18.91 g, 123.32 mmol, 4.0 eq.) were dissolved in acetonitrile (100 mL), and the mixture was reacted at 90° C. for 18 h. The reaction solution was concentrated under reduced pressure, and water (50 mL) and EA (50 mL) were added to the crude product, followed by liquid separation. The aqueous phase was extracted with EA (50 mL×2), and the organic phases were combined, dried and concentrated. The crude product was purified by silica gel column chromatography (PE:EA=10:1) to give the product (2.4 g, yield: 36.4%).

### Step 3: Synthesis of 4-(4-hydroxy-6-methylphthalazin-1-yl)-3-methoxybenzonitrile and 4-(4-hydroxy-7-methylphthalazin-1-yl)-3-methoxybenzonitrile

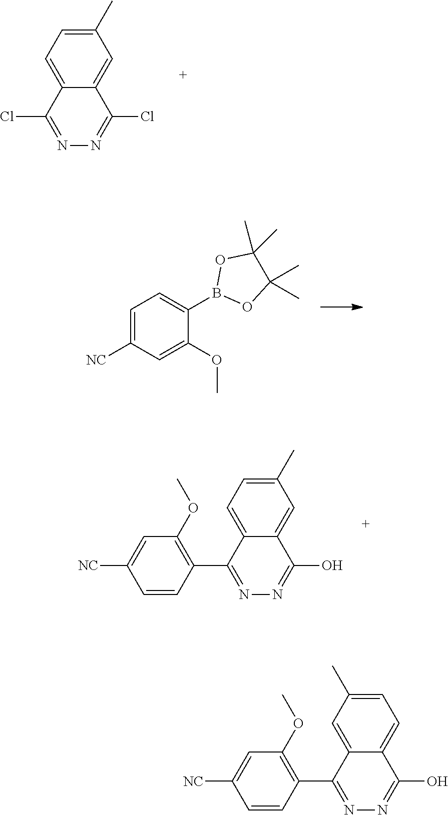 [SMILES: Cc1ccc2c(Cl)nnc(Cl)c2c1 ; COc1cc(C#N)ccc1B1OC(C)(C)C(C)(C)O1 ; COc1cc(C#N)ccc1-c1nnc(O)c2cc(C)ccc12 ; COc1cc(C#N)ccc1-c1nnc(O)c2ccc(C)cc12]

1,4-Dichloro-6-methylphthalazine (1.0 g, 4.69 mmol, 1.0 eq.), 3-methoxy-4-(4,4,5,5-tetramethyl-1,3,2-dioxaborolan-2-yl)benzonitrile (1.46 g, 5.63 mmol, 1.2 eq.), PdCl₂(dppf) (344 mg, 0.47 mmol, 0.1 eq.) and sodium bicarbonate (788 mg, 9.38 mmol, 2.0 eq.) were dissolved in 1,4-dioxane (20 mL) and H₂O (4 mL), and the mixture was reacted at 100° C. for 3 h in nitrogen atmosphere. After the reaction was completed as detected by LC-MS, the reaction solution was concentrated under reduced pressure, and the crude product was purified by silica gel column chromatography (PE:EA=3:1) to give the product (0.4 g, yield: 29.4%).

### Step 4: Synthesis of 4-(4-chloro-6-methylphthalazin-1-yl)-3-methoxybenzonitrile and 4-(4-chloro-7-methylphthalazin-1-yl)-3-methoxybenzonitrile

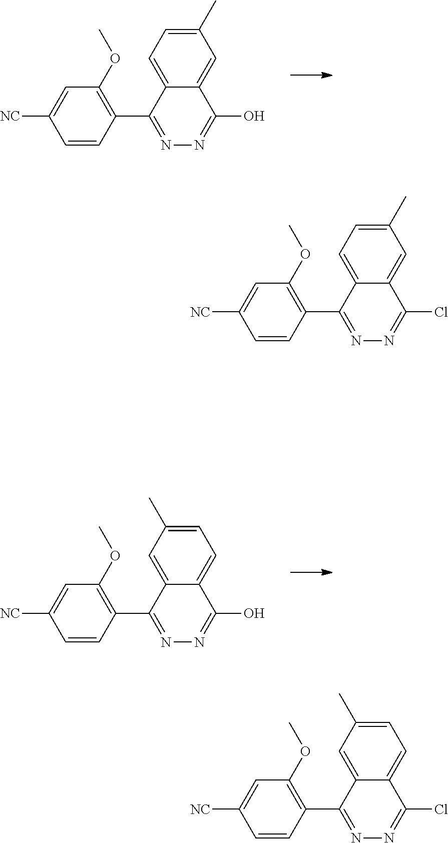 [SMILES: COc1cc(C#N)ccc1-c1nnc(O)c2cc(C)ccc12 ; COc1cc(C#N)ccc1-c1nnc(Cl)c2cc(C)ccc12 ; COc1cc(C#N)ccc1-c1nnc(O)c2ccc(C)cc12 ; COc1cc(C#N)ccc1-c1nnc(Cl)c2ccc(C)cc12]

The mixture (0.4 g, 1.37 mmol, 1.0 eq.) of 4-(4-hydroxy-6-methylphthalazin-1-yl)-3-methoxybenzonitrile and 4-(4-hydroxy-7-methylphthalazin-1-yl)-3-methoxybenzonitrile and phosphorus oxychloride (0.42 g, 2.74 mmol, 1.0 eq.) were dissolved in acetonitrile (100 mL), and the mixture was reacted at 90° C. for 1.5 h. After the reaction was completed as monitored by TLC, the reaction solution was concentrated under reduced pressure, and the crude product was dissolved in EA (10 mL). A saturated sodium bicarbonate solution was added to adjust the pH to 9, followed by liquid separation. The organic phase was dried and concentrated to give the product (0.4 g, yield: 94.3%).

### Step 5: Synthesis of tert-butyl (R)-3-((4-(4-cyano-2-methoxyphenyl)-7-methylphthalazin-1-yl)amino)piperidine-1-carboxylate and tert-butyl (R)-3-((4-(4-cyano-2-methoxyphenyl)-6-methylphthalazin-1-yl)amino)piperidine-1-carboxylate

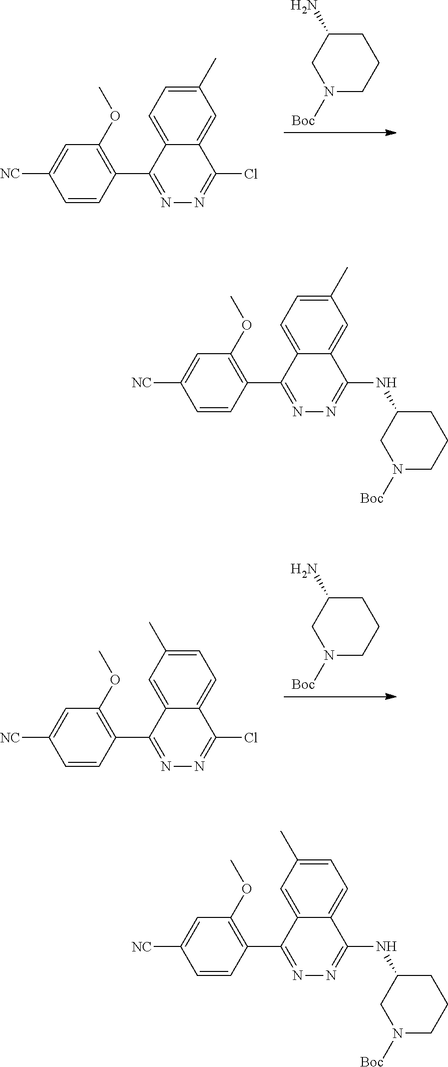 [SMILES: COc1cc(C#N)ccc1-c1nnc(Cl)c2cc(C)ccc12 ; CC(C)(C)OC(=O)N1CCC[C@@H](N)C1 ; COc1cc(C#N)ccc1-c1nnc(N[C@@H]2CCCN(C(=O)OC(C)(C)C)C2)c2cc(C)ccc12 ; COc1cc(C#N)ccc1-c1nnc(Cl)c2ccc(C)cc12 ; CC(C)(C)OC(=O)N1CCC[C@@H](N)C1 ; COc1cc(C#N)ccc1-c1nnc(N[C@@H]2CCCN(C(=O)OC(C)(C)C)C2)c2ccc(C)cc12]

The mixture (380 mg, 1.23 mmol, 1.0 eq.) of 4-(4-chloro-6-methylphthalazin-1-yl)-3-methoxybenzonitrile and 4-(4-chloro-7-methylphthalazin-1-yl)-3-methoxybenzonitrile, tert-butyl (R)-3-aminopiperidine-1-carboxylate (368 mg, 1.84 mmol, 1.5 eq.), Pd₂(dba)₃ (110 mg, 0.12 mmol, 0.1 eq.), BINAP (174 mg, 0.25 mmol, 0.2 eq.) and cesium carbonate (802 mg, 2.46 mmol, 2.0 eq.) were added to 1,4-dioxane (10 mL), and the mixture was reacted at 90° C. for 17 h in nitrogen atmosphere. After the reaction was completed as detected by LC-MS, the reaction solution was concentrated under reduced pressure, and the crude product was purified by silica gel column chromatography (MeOH:DCM=1:50) to give the product (0.3 g, yield: 78.9%).

### Step 6: Synthesis of (R)-3-hydroxy-4-(6-methyl-4-(piperidin-3-ylamino)phthalazin-1-yl)benzonitrile and (R)-3-hydroxy-4-(7-methyl-4-(piperidin-3-ylamino)phthalazin-1-yl)benzonitrile

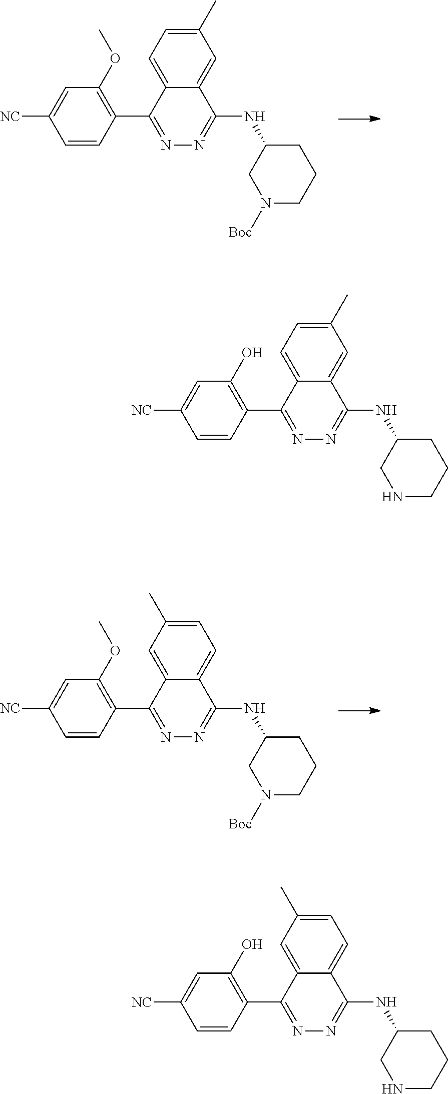 [SMILES: COc1cc(C#N)ccc1-c1nnc(N[C@@H]2CCCN(C(=O)OC(C)(C)C)C2)c2cc(C)ccc12 ; Cc1ccc2c(-c3ccc(C#N)cc3O)nnc(N[C@@H]3CCCNC3)c2c1 ; COc1cc(C#N)ccc1-c1nnc(N[C@@H]2CCCN(C(=O)OC(C)(C)C)C2)c2ccc(C)cc12 ; Cc1ccc2c(N[C@@H]3CCCNC3)nnc(-c3ccc(C#N)cc3O)c2c1]

The mixture (300 mg, 0.63 mmol, 1.0 eq.) of tert-butyl (R)-3-((4-(4-cyano-2-methoxyphenyl)-7-methylphthalazin-1-yl)amino)piperidine-1-carboxylate and tert-butyl (R)-3-((4-(4-cyano-2-methoxyphenyl)-6-methylphthalazin-1-yl)amino)piperidine-1-carboxylate was dissolved in DCM (5 mL), followed by dropwise addition of a 1 mol/L solution of boron tribromide in dichloromethane (1.9 mL, 1.89 mmol, 3.0 eq.) at room temperature. The mixture was reacted at room temperature for 3 h. After the reaction was completed as detected by LC-MS, an appropriate amount of methanol was added to the system to quench the reaction, and water (10 mL) was added, followed by liquid separation. The pH of the aqueous phase was adjusted to 9 with sodium bicarbonate, the aqueous phase was extracted with DCM (15 mL×3), and the organic phase was dried and concentrated to give the product (150 mg, yield: 66.3%).

### Step 7: Synthesis of (R)-3-hydroxy-4-(6-methyl-4-((1-methylpiperidin-3-yl)amino)phthalazin-1-yl)benzonitrile (Compound 55) and (R)-3-hydroxy-4-(7-methyl-4-((1-methylpiperidin-3-yl)amino)phthalazin-1-yl)benzonitrile (Compound 54)

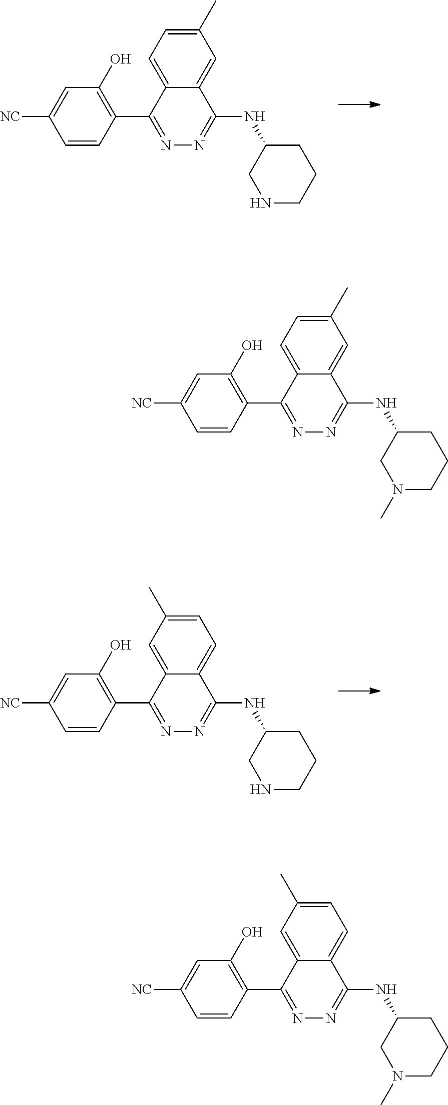 [SMILES: Cc1ccc2c(-c3ccc(C#N)cc3O)nnc(N[C@@H]3CCCNC3)c2c1 ; Cc1ccc2c(-c3ccc(C#N)cc3O)nnc(N[C@@H]3CCCN(C)C3)c2c1 ; Cc1ccc2c(N[C@@H]3CCCNC3)nnc(-c3ccc(C#N)cc3O)c2c1 ; Cc1ccc2c(N[C@@H]3CCCN(C)C3)nnc(-c3ccc(C#N)cc3O)c2c1]

The mixture (150 mg, 0.42 mmol, 1.0 eq.) of (R)-3-hydroxy-4-(6-methyl-4-(piperidin-3-ylamino)phthalazin-1-yl)benzonitrile and (R)-3-hydroxy-4-(7-methyl-4-(piperidin-3-ylamino)phthalazin-1-yl)benzonitrile and an aqueous formaldehyde solution (37%) (34 mg, 0.42 mmol, 1.0 eq.) were dissolved in methanol (3 mL), and the mixture was stirred at room temperature for 5 min, followed by addition of sodium cyanoborohydride (26 mg, 0.42 mmol, 1.0 eq.). The resulting mixture was reacted at room temperature for 5 min. After the reaction was completed as monitored by LC-MS, the reaction solution was concentrated under reduced pressure, the crude product was dissolved in DCM (10 mL), and water (10 mL) was added, followed by liquid separation. The organic phase was dried and concentrated, and the crude product was purified by preparative thin-layer chromatography (dichloromethane:methanol:ammonia solution=10:1:0.5) to give the product (R)-3-hydroxy-4-(6-methyl-4-((1-methylpiperidin-3-yl)amino)phthalazin-1-yl)benzonitrile with a larger Rf value (23 mg, yield: 14.7%) (compound 55).

¹HNMR (400 MHz, DMSO-d6) δ (ppm): 10.48 (s, 1H), 8.21 (s, 1H), 7.62-7.60 (d, 1H), 7.49-7.47 (d, 1H), 7.41-7.39 (t, 1H), 7.34-7.32 (d, 2H), 7.04-7.02 (d, 1H), 4.42-4.40 (t, 1H), 3.08-3.06 (d, 1H), 2.72-2.69 (d, 1H), 2.53 (s, 3H), 2.21 (s, 3H), 1.99-1.90 (m, 3H), 1.78-1.74 (m, 1H), 1.62-1.59 (d, 1H), 1.49-1.43 (m, 1H).

The two-dimensional NOE spectrum showed coupling signals between 7.49-7.47 (d, 1H) and 7.34-7.32 (d, 1H) and coupling signals between 8.21 (s, 1H) and 7.04-7.02 (d, 1H).

Molecular formula: C₂₂H₂₆N₄O Precise molecular weight: 373.19 LC-MS (Pos, m/z)=374.14[M+H]⁺.

Simultaneously, the product (R)-3-hydroxy-4-(7-methyl-4-((1-methylpiperidin-3-yl)amino)phthalazin-1-yl)benzonitrile with a smaller Rf value (20 mg, yield: 12.8%) (compound 54) was also given.

¹HNMR (400 MHz, DMSO-d6) δ (ppm): 10.43 (s, 1H), 8.31-8.29 (d, 1H), 7.70-7.68 (d, 1H), 7.48-7.46 (d, 1H), 7.42-7.39 (m, 1H), 7.32 (s, 1H), 7.19 (s, 1H), 7.08-7.06 (d, 1H), 4.41-4.39 (t, 1H), 3.08-3.06 (d, 1H), 2.72-2.69 (d, 1H), 2.42 (s, 3H), 2.21 (s, 3H), 1.98-1.89 (m, 3H), 1.76-1.73 (t, 1H), 1.62-1.59 (d, 1H), 1.49-1.43 (m, 1H).

The two-dimensional NOE spectrum showed coupling signals between 10.43 (s, 1H) and 7.19 (s, 1H), between 8.31-8.29 (d, 1H) and 7.08-7.06 (d, 1H), and between 8.31-8.29 (d, 1H) and 7.70-7.68 (d, 1H).

Molecular formula: C₂₂H₂₆N₄O Precise molecular weight: 373.19 LC-MS (Pos, m/z)=374.13[M+H]⁺.

### Example 33: Synthesis of (R)-5-methyl-2-(4-((1-methylpiperidin-3-yl)amino)-6-(trifluoromethyl)phthalazin-1-yl)phenol (Compound 24)

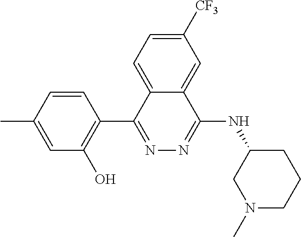 [SMILES: Cc1ccc(-c2nnc(N[C@@H]3CCCN(C)C3)c3cc(C(F)(F)F)ccc23)c(O)c1]

### Step 1: Synthesis of methyl 2-bromo-5-(trifluoromethyl)benzoate

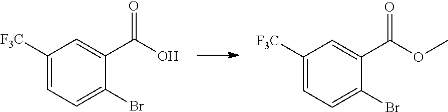

2-Bromo-5-(trifluoromethyl)benzoic acid (10.0 g, 37.17 mmol, 1.0 eq.), iodomethane (7.91 g, 55.76 mmol, 1.5 eq.) and potassium carbonate (7.69 g, 55.76 mmol, 1.5 eq.) were dissolved in DMF (50 mL), and the mixture was reacted at room temperature for 1 h. After no materials were left as monitored by TLC, the reaction solution was poured into water (50 mL), followed by extraction with MTBE (50 mL×2). The organic phase was dried and concentrated to give the product (10.52 g, yield: 100%).

### Step 2: Synthesis of methyl 2-(2-methoxy-4-methylbenzoyl)-5-(trifluoromethyl)benzoate

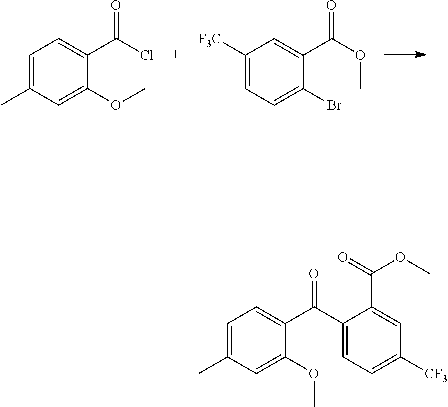

Methyl 2-bromo-5-(trifluoromethyl)benzoate (8.0 g, 28.26 mmol, 1.0 eq.) was dissolved in THE (50 mL), and the mixture was cooled to −30° C., followed by dropwise and slow addition of a solution of an isopropylmagnesium chloride lithium chloride complex in tetrahydrofuran (1.3 mol/L, 26 mL, 33.91 mmol, 1.2 eq.). The resulting mixture was stirred for 2 h. The system was cooled to −60° C., and a solution of 2-methoxy-4-methylbenzoyl chloride (5.21 g, 28.26 mmol, 1.0 eq.) in THE (50 mL) was added dropwise to the system. The mixture was reacted for 1 h. After the generation of the product as monitored by LC-MS, the reaction solution was poured into a saturated aqueous potassium carbonate solution (200 mL), followed by extraction with EA (100 mL×2). The organic phase was dried and concentrated to give the product (5.0 g, yield: 50.2%).

### Step 3: Synthesis of 4-(2-methoxy-4-methylphenyl)-7-(trifluoromethyl)phthalazin-1-ol

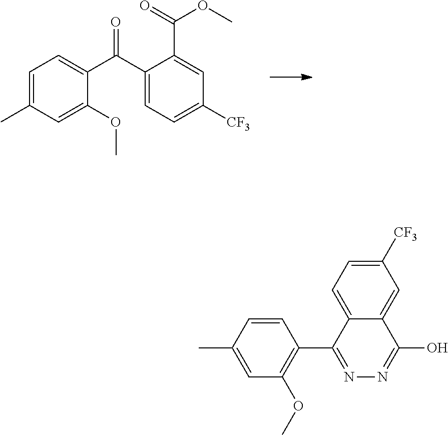 [SMILES: COC(=O)c1cc(C(F)(F)F)ccc1C(=O)c1ccc(C)cc1OC ; COc1cc(C)ccc1-c1nnc(O)c2cc(C(F)(F)F)ccc12]

Methyl 2-(2-methoxy-4-methylbenzoyl)-5-(trifluoromethyl)benzoate (5.0 g, 14.19 mmol, 1.0 eq.) and 85% hydrazine hydrate (1.25 g, 21.28 mmol, 1.5 eq.) were dissolved in ethanol (50 mL), and the mixture was reacted at 80° C. for 17 h. After the reaction was completed as monitored by LC-MS, the reaction solution was concentrated under reduced pressure, and the crude product was purified by silica gel column chromatography (MEOH:DCM=1:100) to give the product (1.1 g, yield: 23.2%).

### Step 4: Synthesis of 4-chloro-1-(2-methoxy-4-methylphenyl)-6-(trifluoromethyl)phthalazine

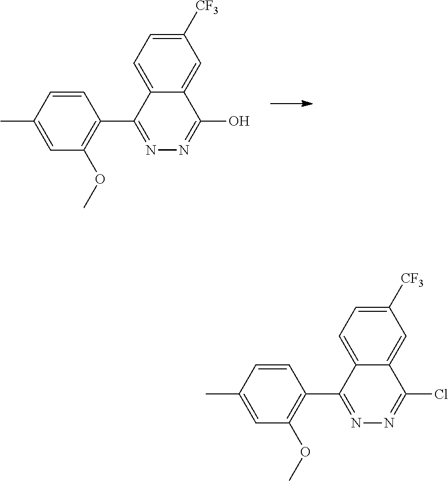 [SMILES: COc1cc(C)ccc1-c1nnc(O)c2cc(C(F)(F)F)ccc12 ; COc1cc(C)ccc1-c1nnc(Cl)c2cc(C(F)(F)F)ccc12]

4-(2-Methoxy-4-methylphenyl)-7-(trifluoromethyl)phthalazin-1-ol (1.0 g, 2.99 mmol, 1.0 eq.) and phosphorus oxychloride (917 mg, 5.98 mmol, 2.0 eq.) were dissolved in acetonitrile (20 mL), and the mixture was reacted at 90° C. for 1 h. After the reaction was completed as monitored by TLC, the reaction solution was concentrated under reduced pressure, and the crude product was purified by silica gel column chromatography (PE:EA=10:1) to give the product (0.5 g, yield: 47.6%).

### Step 5: Synthesis of tert-butyl (R)-3-((4-(2-methoxy-4-methylphenyl)-7-(trifluoromethyl)phthalazin-1-yl)amino)piperidine-1-carboxylate

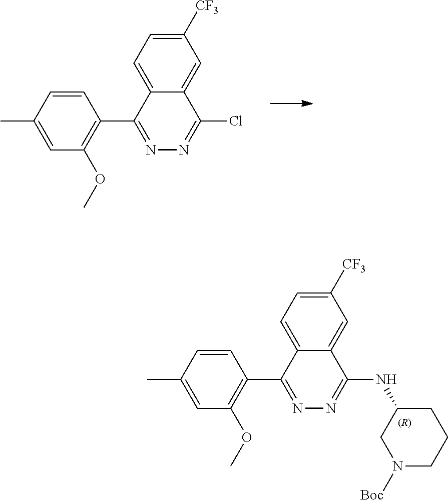

4-Chloro-1-(2-methoxy-4-methylphenyl)-6-(trifluoromethyl)phthalazine (0.5 g, 1.41 mmol, 1.0 eq.), tert-butyl (R)-3-aminopiperidine-1-carboxylate (424 mg, 2.12 mmol, 1.5 eq.), Pd₂(dba)₃ (128 mg, 0.14 mmol, 0.1 eq.), BINAP (174 mg, 0.28 mmol, 0.2 eq.) and cesium carbonate (919 mg, 2.82 mmol, 2.0 eq.) were added to 1,4-dioxane (10 mL), and the mixture was reacted at 90° C. for 21 h in nitrogen atmosphere. After the reaction was completed as detected by LC-MS, the reaction solution was concentrated under reduced pressure, and the crude product was purified by silica gel column chromatography (PE:EA=2:1s) to give the product (0.3 g, yield: 41.2%).

### Step 6: Synthesis of (R)-5-methyl-2-(4-(piperidin-3-ylamino)-6-(trifluoromethyl)phthalazin-1-yl)phenol

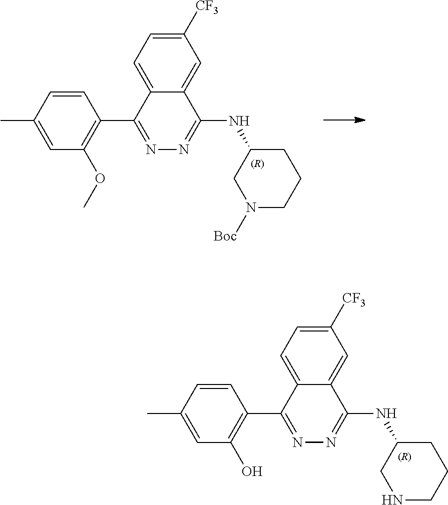

Tert-butyl (R)-3-((4-(2-methoxy-4-methylphenyl)-7-(trifluoromethyl)phthalazin-1-yl)amino)piperidine-1-carboxylate (0.3 g, 0.58 mmol, 1.0 eq.) was dissolved in DCM (5 mL), followed by dropwise addition of a 1 mol/L solution of boron tribromide in dichloromethane (1.7 mL, 1.74 mmol, 3.0 eq.). The mixture was reacted for 1 h. After the reaction was completed as detected by LC-MS, an appropriate amount of methanol was added to the system to quench the reaction, and water (5 mL) was added, followed by liquid separation. The pH of the aqueous phase was adjusted to 9 with sodium bicarbonate, followed by extraction with DCM (10 mL×3). The organic phase was dried and concentrated to give the product (100 mg, yield: 42.9%).

### Step 7: Synthesis of (R)-5-methyl-2-(4-((1-methylpiperidin-3-yl)amino)-6-(trifluoromethyl)phthalazin-1-yl)phenol

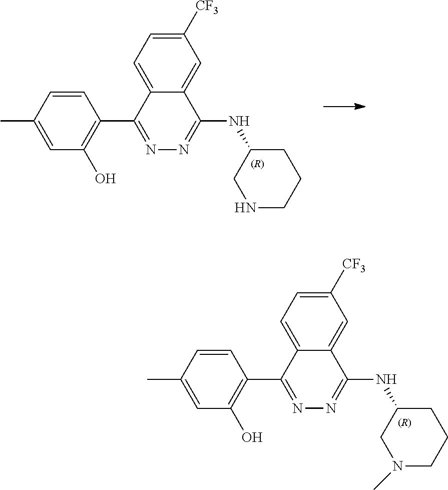

(R)-5-methyl-2-(4-(piperidin-3-ylamino)-6-(trifluoromethyl)phthalazin-1-yl)phenol (100 mg, 0.24 mmol, 1.0 eq.) was dissolved in methanol (2 mL), followed by addition of an aqueous formaldehyde solution (37%) (19 mg, 0.24 mmol, 1.0 eq.). The mixture was stirred at room temperature for 5 min, followed by addition of sodium cyanoborohydride (15 mg, 0.24 mmol, 1.0 eq.). The mixture was reacted at room temperature for 5 min. After the reaction was completed as monitored by LC-MS, the reaction solution was concentrated under reduced pressure. The crude product was purified by preparative thin-layer chromatography (DCM:MeOH=8:1) to give the product (20 mg, yield: 20%).

¹HNMR (300 MHz, DMSO-d6) δ (ppm): 9.56 (s, 1H), 8.90 (s, 1H), 8.09-8.06 (d, 1H), 7.68-7.66 (d, 1H), 7.50-7.47 (d, 1H), 7.20-7.18 (d, 1H), 6.82-6.78 (d, 1H), 4.46-4.44 (d, 1H), 3.17-3.13 (d, 1H), 2.34 (s, 3H), 2.26 (s, 1H), 2.02-1.98 (d, 3H), 1.82-1.77 (d, 1H), 1.65-1.62 (d, 1H), 1.51-1.45 (m, 1H).

Molecular formula: C₂₂H₂₃F₃N₄O Precise molecular weight: 416.18 LC-MS (Pos, m/z)=417.13[M+H]⁺.

### Example 34: Synthesis of (R)-5-methyl-2-(4-((1-methylpiperidin-3-yl)oxy)phthalazin-1-yl)phenol (Compound 130)

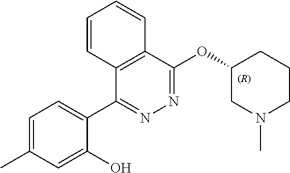

### Step 1: Synthesis of tert-butyl (R)-3-((4-(2-hydroxy-4-methylphenyl)phthalazin-1-yl)oxy)piperidine-1-carboxylate

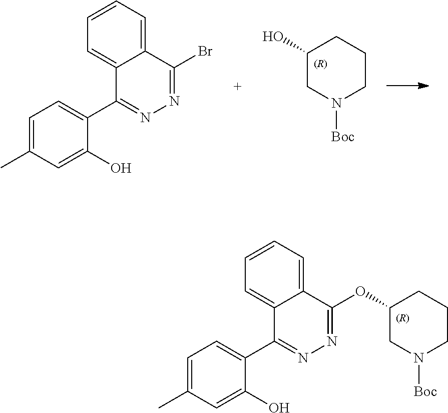

Tert-butyl (R)-3-hydroxypiperidine-1-carboxylate (287.3 mg, 1.42 mmol, 1.5 eq.) was added to anhydrous N,N-dimethylformamide (3.0 mL), followed by addition of sodium hydride (60%) (56.8 mg, 1.42 mmol, 1.5 eq.) under an ice bath. The mixture was reacted for 0.5 h in nitrogen atmosphere, followed by dropwise addition of a solution of 2-(4-bromophthalazin-1-yl)-5-methylphenol (300.0 mg, 0.95 mmol, 1.0 eq.) in N,N-dimethylformamide (5.0 mL). The resulting mixture was reacted at room temperature for 12 h. After the reaction was completed as monitored by TLC, water (50.0 mL) was added to the system to quench the reaction, followed by extraction with ethyl acetate (100.0 mL). The organic phase was dried over anhydrous sodium sulfate and filtered, and the filtrate was concentrated under reduced pressure to give the crude product (360.0 mg, used directly in the next step).

### Step 2: Synthesis of (R)-5-methyl-2-(4-(piperidin-3-yloxy)phthalazin-1-yl)phenol

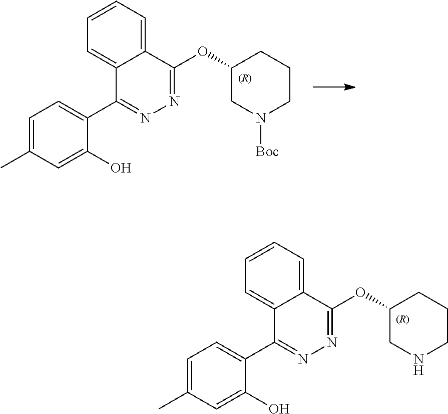

Tert-butyl (R)-3-((4-(2-hydroxy-4-methylphenyl)phthalazin-1-yl)oxy)piperidine-1-carboxylate (360.0 mg, crude product, 0.82 mmol, 1.0 eq.) was added to dichloromethane (5.0 mL), followed by dropwise addition of trifluoroacetic acid (5.0 mL). The mixture was reacted at room temperature for 2 h. After the reaction was completed as monitored by TLC, a saturated aqueous sodium bicarbonate solution was added to the system to adjust the pH to 7-8, followed by extraction with dichloromethane (50.0 mL). The organic phase was dried over anhydrous sodium sulfate and filtered, and the filtrate was concentrated under reduced pressure. The crude product was purified by preparative thin-layer chromatography (specification of silica gel: 100-200 mesh, dichloromethane:methanol=10:1) to give the product (51.0 mg, yield: 18.5%).

### Step 3: Synthesis of (R)-5-methyl-2-(4-((1-methylpiperidin-3-yl)oxy)phthalazin-1-yl)phenol

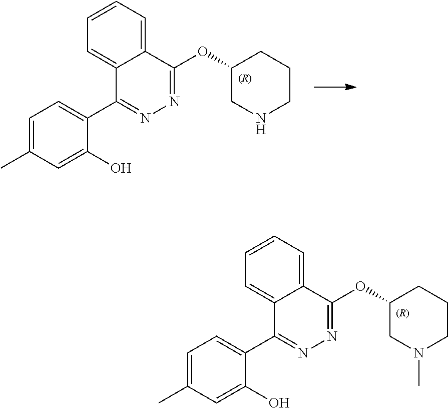

(R)-5-methyl-2-(4-(piperidin-3-yloxy)phthalazin-1-yl)phenol (51.0 mg, 0.15 mmol, 1.0 eq.) and an aqueous formaldehyde solution (37%) (14.8 mg, 0.18 mmol, 1.2 eq.) were added to methanol (5.0 mL), and the mixture was stirred at room temperature for 0.5 h, followed by addition of sodium cyanoborohydride (13.2 mg, 0.21 mmol, 1.4 eq.). The resulting mixture was reacted at room temperature for 2 h. After the reaction was completed as monitored by TLC, the reaction solution was concentrated under reduced pressure, followed by addition of a saturated aqueous sodium bicarbonate solution (100.0 mL). The mixture was stirred for 0.5 h and extracted with dichloromethane (100.0 mL). The organic phase was dried over anhydrous sodium sulfate and filtered, and the filtrate was concentrated under reduced pressure. The crude product was purified by preparative thin-layer chromatography (dichloromethane:methanol=10:1) to give the product (30.0 mg, yield: 57.2%).

¹HNMR (300 MHz, DMSO-d6) δ (ppm): 9.58 (s, 1H), 8.22-8.20 (d, J=6 Hz, 1H), 7.98-7.87 (m, 2H), 7.61-7.58 (m, 1H), 7.20-7.18 (m, 1H), 6.84-6.79 (m, 2H), 5.49 (s, 1H), 3.36-3.34 (m, 1H), 3.04-3.01 (m, 1H), 2.55-2.49 (m, 2H), 2.35 (s, 3H), 2.26 (s, 3H), 1.87-1.66 (m, 4H).

Molecular formula: C₂₁H₂₃N₃O₂ Precise molecular weight: 349.18 LC-MS (m/z)=350.17[M+H]⁺.

### Example 35: Synthesis of (R)-5-cyclopropyl-2-(4-((1-methylpiperidin-3-yl)amino)phthalazin-1-yl)phenol (Compound 72)

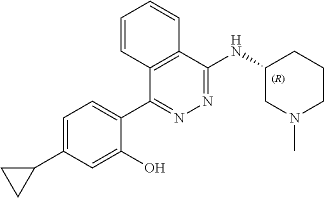

### Step 1: Synthesis of (R)-4-(4-cyclopropyl-2-methoxyphenyl)-N-(1-methylpiperidin-3-yl)phthalazin-1-amine

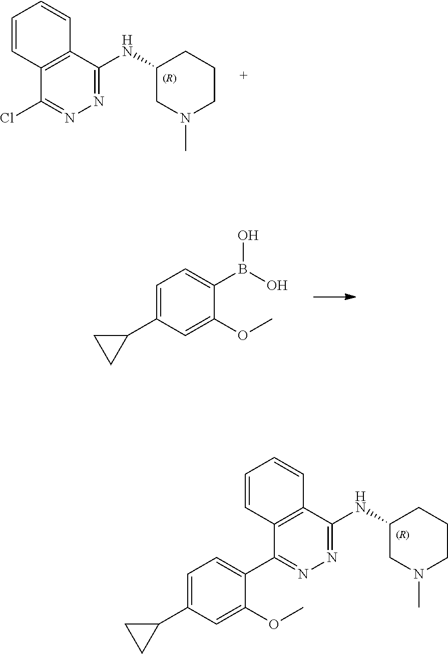

(R)-4-chloro-N-(1-methylpiperidin-3-yl)phthalazin-1-amine (350.0 mg, 1.26 mmol, 1.0 eq.), (4-cyclopropyl-2-methoxyphenyl)boronic acid (364.2 mg, 1.89 mmol, 1.5 eq.), sodium bicarbonate (317.5 mg, 3.78 mmol, 3.0 eq.) and Pd(PPh₃)₄ (72.8 mg, 0.06 mmol, 0.05 eq.) were added to a mixed solution of 1,4-dioxane (12.0 mL) and water (6.0 mL), and the mixture was reacted at 110° C. for 12 h in nitrogen atmosphere. After the reaction was completed as monitored by TLC, the reaction solution was washed with water (50.0 mL), followed by extraction with ethyl acetate (100.0 mL). The organic phase was dried over anhydrous sodium sulfate and filtered, and the filtrate was concentrated under reduced pressure. The crude product was purified by silica gel column chromatography (specification of silica gel: 100-200 mesh, dichloromethane:methanol=40:1 to 10:1) to give the product (387.0 mg, yield: 79.1%).

### Step 2: Synthesis of (R)-5-cyclopropyl-2-(4-((1-methylpiperidin-3-yl)amino)phthalazin-1-yl)phenol

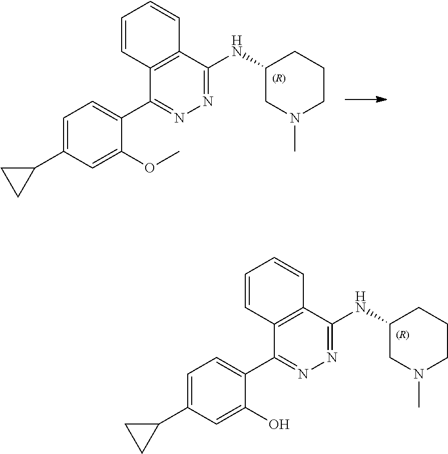

(R)-4-(4-cyclopropyl-2-methoxyphenyl)-N-(1-methylpiperidin-3-yl)phthalazin-1-amine (387.0 mg, 0.99 mmol, 1.0 eq.) was added to dichloromethane (5.0 mL), followed by dropwise addition of boron tribromide (748.6 mg, 2.98 mmol, 3.0 eq.) under an ice bath. The reaction mixture was warmed to room temperature and reacted for 2 h. After the reaction was completed as monitored by TLC, methanol (10.0 mL) was added to the system under an ice bath to carefully quench the reaction, followed by concentration under reduced pressure, addition of dichloromethane (100.0 mL) and back extraction with water (50.0 mL). The pH of the aqueous phase was adjusted to 7-8 with a saturated aqueous sodium bicarbonate solution, followed by extraction with dichloromethane (100.0 mL). The organic phase was dried over anhydrous sodium sulfate and filtered, and the filtrate was concentrated under reduced pressure. The crude product was purified by preparative thin-layer chromatography to give the product (160.0 mg, yield: 43.2%).

¹HNMR (300 MHz, DMSO-d6) δ (ppm): 9.66 (s, 1H), 8.47-8.45 (d, J=6 Hz, 1H), 7.88-7.76 (m, 2H), 7.55-7.53 (m, 1H), 7.32 (s, 1H), 7.17-7.15 (m, 1H), 6.70-6.66 (m, 2H), 4.59 (s, 1H), 3.06-3.03 (m, 2H), 2.55 (s, 3H), 2.01-1.70 (m, 6H), 1.02-0.96 (m, 2H), 0.88-0.84 (m, 1H), 0.72-0.67 (m, 2H).

Molecular formula: C₂₃H₂₆N₄O Precise molecular weight: 374.21 LC-MS (m/z)=375.16[M+H]⁺.

### Example 36: Synthesis of Compound (R)-5-cyclopropyl-2-(4-((1-methylpiperidin-3-yl)amino)pyrido[3,4-d]pyridazin-1-yl)phenol (Compound 95)

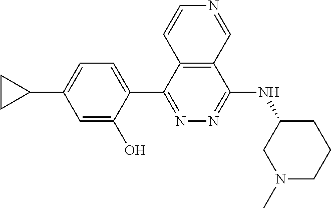 [SMILES: CN1CCC[C@@H](Nc2nnc(-c3ccc(C4CC4)cc3O)c3ccncc23)C1]

### Step 1: Synthesis of Intermediate 4-cyclopropyl-2-methoxybenzoyl chloride

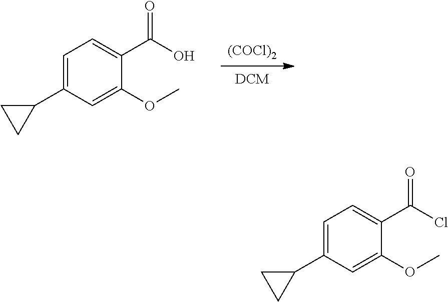

4-Cyclopropyl-2-methoxybenzoic acid (5 g, 26.01 mmol, 1.0 eq.) was dissolved in dichloromethane (50 mL), and N,N-dimethylformamide (0.05 mL) was added dropwise, followed by dropwise and slow addition of oxalyl chloride (9.9 g, 78.03 mmol, 3.0 eq.). After the completion of dropwise addition, the mixture was reacted at room temperature for 20 min. After the reaction was completed as detected by TLC, the reaction solution was concentrated and directly used in the next step.

### Step 2: Synthesis of Intermediate methyl 2-chloro-4-(4-cyclopropyl-2-methoxybenzoyl)nicotinate

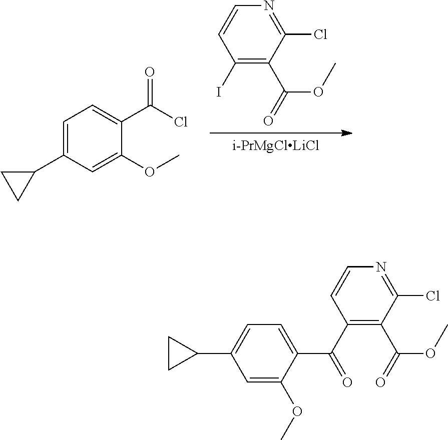 [SMILES: "i-PrMgCl·LiCl" ; COc1cc(C2CC2)ccc1C(=O)Cl ; COC(=O)c1c(I)ccnc1Cl ; COC(=O)c1c(C(=O)c2ccc(C3CC3)cc2OC)ccnc1Cl]

4-Cyclopropyl-2-methoxybenzoyl chloride (7.74 g, 26.01 mmol, 1.0 eq.) was dissolved in tetrahydrofuran (80 mL), followed by purge with nitrogen. A 1.3 mol/L solution of an isopropylmagnesium chloride lithium chloride complex in tetrahydrofuran (22 mL, 28.61 mmol, 1.1 eq.) was added dropwise and slowly at −40° C. After the completion of dropwise addition, the mixture was stirred at −40° C. for 20 min. Cuprous cyanide (2.33 g, 26.01 mmol, 1.0 eq.) was then added, and the mixture was reacted at −40° C. for 30 min. A solution of 4-cyclopropyl-2-methoxybenzoyl chloride (26.01 mmol, 1.0 eq.) in tetrahydrofuran (50 mL) was then added dropwise. After the completion of dropwise addition, the mixture was reacted at 5° C. for 1 h. After the reaction was completed as detected by TLC, saturated brine (100 mL) and a saturated aqueous sodium bicarbonate solution (50 mL) were added to the reaction solution, followed by extraction with ethyl acetate (50 mL×3). The organic phases were combined, dried over anhydrous sodium sulfate and concentrated to give the product (7.37 g, two-step yield: 82%).

### Step 3: Synthesis of Intermediate methyl 4-(4-cyclopropyl-2-methoxybenzoyl)nicotinate

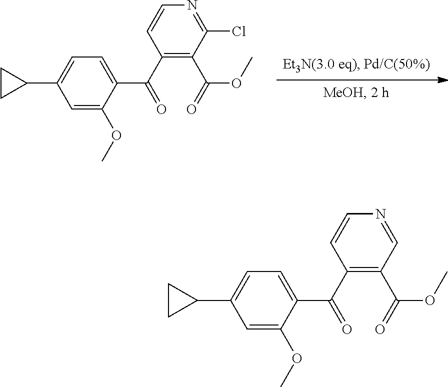 [SMILES: COC(=O)c1c(C(=O)c2ccc(C3CC3)cc2OC)ccnc1Cl ; "Et3N(3.0 eq), Pd/C(50%)" ; "MeOH, 2 h" ; COC(=O)c1cnccc1C(=O)c1ccc(C2CC2)cc1OC]

Methyl 2-chloro-4-(4-cyclopropyl-2-methoxybenzoyl)nicotinate (7.37 g, 21.32 mmol, 1.0 eq.) was dissolved in methanol (100 mL), followed by addition of triethylamine (6.47 g, 63.96 mmol, 2.0 eq.) and 5% Pd/C (3.6 g) and purge with hydrogen. The mixture was reacted at room temperature of 25° C. for 1 h. After the reaction was completed as detected by TLC, the reaction solution was filtered through celite, and the filtrate was concentrated and purified by silica gel column chromatography (ethyl acetate:petroleum ether=1:5) to give the product (4.06 g, yield: 61.1%).

### Step 4: Synthesis of Intermediate 1-(4-cyclopropyl-2-methoxyphenyl)pyrido[3,4-d]pyridazin-4(3H)-one

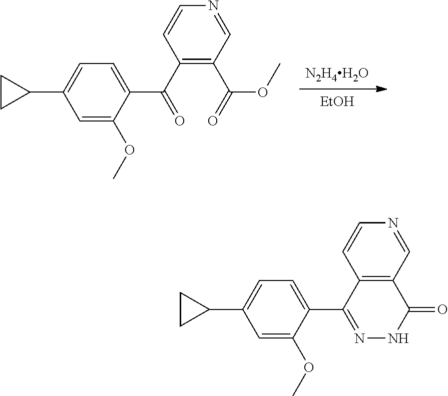

Methyl 4-(4-cyclopropyl-2-methoxybenzoyl)nicotinate (4.06 g, 13.01 mmol, 1.0 eq.) was added to ethanol (50 mL), followed by addition of 85% hydrazine hydrate (1.53 g, 26.02 mmol, 2.0 eq.). The mixture was heated to 90° C. and reacted for 2 h. After the reaction was completed as detected by TLC, the reaction solution was filtered, and the filter cake was rinsed with ethanol (5 mL) and dried to give the product (2.44 g, yield: 64%). (2100054-002).

### Step 5: Synthesis of Intermediate 4-chloro-1-(4-cyclopropyl-2-methoxyphenyl)pyrido[3,4-d]pyridazine

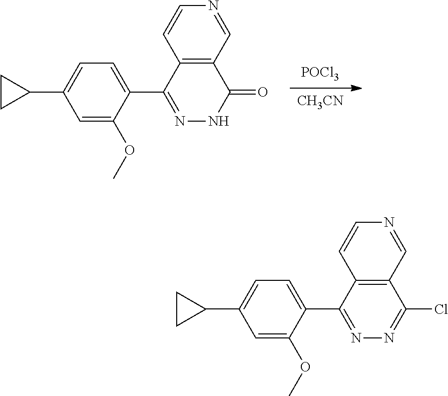

1-(4-Cyclopropyl-2-methoxyphenyl)pyrido[3,4-d]pyridazin-4(3H)-one (2.44 g, 8.32 mmol, 1.0 eq.) was added to acetonitrile (30 mL), followed by addition of phosphorus oxychloride (2.55 g, 16.64 mmol, 2.0 eq.). The mixture was reacted at 100° C. for 16 h. After the reaction was completed as detected by TLC, the reaction solution was concentrated and dissolved in dichloromethane (20 mL). The solution was added dropwise to a saturated aqueous sodium bicarbonate solution (100 mL), followed by extraction. The aqueous phase was extracted with dichloromethane (50 mL×3), and the organic phases were combined, washed with saturated brine (50 mL), dried over anhydrous magnesium sulfate and concentrated. The crude product was purified by silica gel column chromatography (ethyl acetate:petroleum ether=1:10 to 1:5) to give the product (1.94 g, yield: 74.9%).

### Step 6: Synthesis of Intermediate 2-(4-chloropyrido[3,4-d]pyridazin-1-yl)-5-cyclopropylphenol

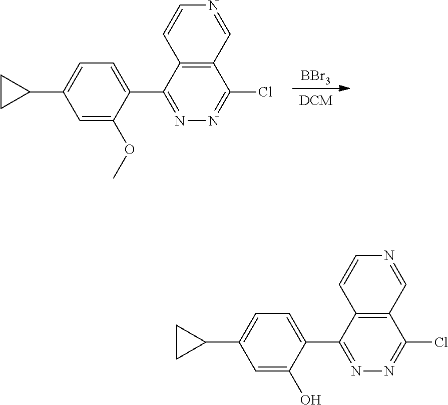

4-Chloro-1-(4-cyclopropyl-2-methoxyphenyl)pyrido[3,4-d]pyridazine (1.94 g, 6.22 mmol, 1.0 eq.) was dissolved in dichloromethane (20 mL), followed by dropwise and slow addition of boron tribromide (4.67 g, 18.66 mmol, 3.0 eq.) at 0° C. After the completion of dropwise addition, the mixture was reacted at room temperature of 25° C. for 30 min. After the reaction was completed as detected by TLC, the reaction solution was added dropwise and slowly to a saturated aqueous sodium bicarbonate solution (50 mL), followed by extraction with dichloromethane (20 mL×3). The organic phases were combined, dried over anhydrous magnesium sulfate and concentrated to give the product (1.2 g).

### Step 7: Synthesis of Intermediate tert-butyl (R)-3-((1-(4-cyclopropyl-2-methoxyphenyl)pyrido[3,4-d]pyridazin-4-yl)amino)piperidine-1-carboxylate

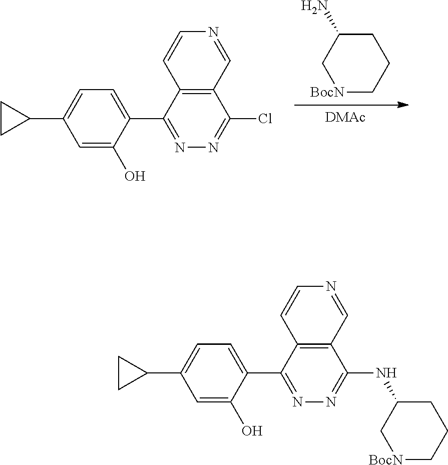

2-(4-Chloropyrido[3,4-d]pyridazin-1-yl)-5-cyclopropyl-phenol (1.2 g, 4.03 mmol, 1.0 eq.) was dissolved in N,N-dimethylacetamide (12 mL), followed by addition of (R)-1-tert-butoxycarbonyl-3-aminopiperidine (1.61 g, 8.06 mmol, 2.0 eq.). The mixture was reacted at 120° C. for 16 h. After the reaction was completed as detected by TLC, the reaction solution was poured into water (20 mL), followed by extraction with ethyl acetate (20 mL). The organic phase was dried over anhydrous sodium sulfate and concentrated. The crude product was subjected to silica gel column chromatography (ethyl acetate:petroleum ether=1:3 to 1:1) to give the product (1.19 g, two-step yield: 41.4%).

### Step 8: Synthesis of Intermediate (R)-5-cyclopropyl-2-(4-(piperidin-3-ylamino)pyrido[3,4-d]pyridazin-1-yl)phenol

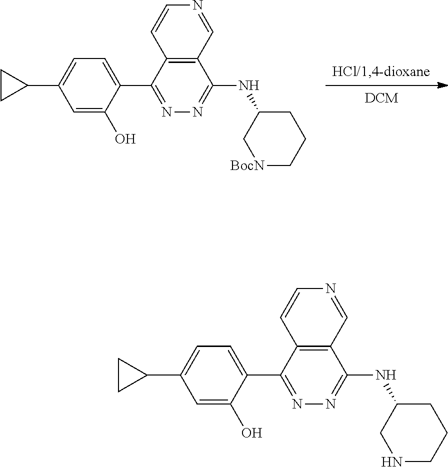

Tert-butyl (R)-3-((1-(4-cyclopropyl-2-methoxyphenyl)pyrido[3,4-d]pyridazin-4-yl)amino)piperidine-1-carboxylate (1.19 g, 2.58 mmol, 1.0 eq.) was dissolved in dichloromethane (3 mL), and the resulting solution was added dropwise to a 4 moL/L solution of hydrogen chloride in 1,4-dioxane (15 mL). After the completion of dropwise addition, the mixture was reacted at room temperature for 5 min. After the reaction was completed as detected by TLC, water (15 mL) was added to the reaction solution, and the pH was adjusted to low alkalinity with sodium bicarbonate, followed by extraction with a mixed solvent of methanol and dichloromethane (1:5, 30 mL×3). The organic phases were combined, dried over anhydrous magnesium sulfate and concentrated, and the crude product was directly used in the next step.

### Step 9: Synthesis of compound (R)-5-cyclopropyl-2-(4-((1-methylpiperidin-3-yl)amino)pyrido[3,4-d]pyridazin-1-yl)phenol

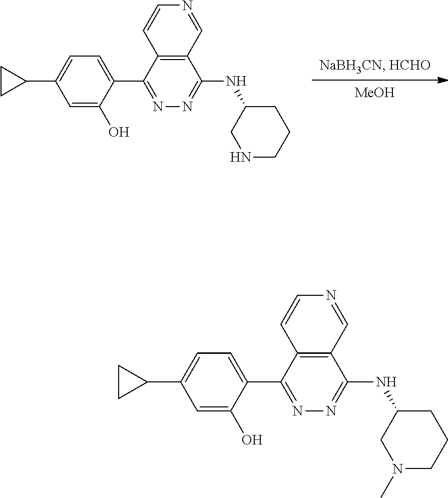

(R)-5-cyclopropyl-2-(4-(piperidin-3-ylamino)pyrido[3,4-d]pyridazin-1-yl)phenol (2.58 mmol, 1.0 eq.) was dissolved in methanol (10 mL), followed by addition of an aqueous formaldehyde solution (37%) (210 mg, 2.58 mmol, 1.0 eq.). The mixture was stirred at room temperature for 5 min, followed by addition of sodium cyanoborohydride (179 mg, 2.84 mmol, 1.1 eq.). The resulting mixture was reacted at room temperature for 5 min. After the reaction was completed as detected by TLC, the reaction solution was concentrated, and saturated brine (2 mL) was added, followed by extraction with a 7 mol/L solution of ammonia in methanol/dichloromethane (1:7, 10 mL×3). The organic phases were combined, dried and concentrated, and the crude product was purified by preparative thin-layer chromatography to give the product (265 mg, two-step yield: 27%).

¹H-NMR (400 MHz, DMSO-d6) δ (ppm): 9.79 (s, 1H), 9.65 (s, 1H), 8.85-8.84 (d, 1H), 7.63-7.61 (d, 1H), 7.34-7.32 (d, 1H), 7.20-7.18 (d, 1H), 6.71-6.68 (m, 2H), 4.51 (s, 1H), 3.22 (s, 1H), 2.85-2.83 (d, 1H), 2.30 (s, 3H), 2.12-2.01 (m, 2H), 1.97-1.90 (m, 1H), 1.81-1.52 (m, 4H), 1.02-0.97 (m, 2H), 0.72-0.68 (m, 2H).

Molecular formula: C₂₂H₂₅N₅O Precise molecular weight: 375.21 LC-MS (m/z): 376.12 [M+H]⁺

### Example 37: Synthesis of (R)-4-(4-((1-cyclopropylpiperidin-3-yl)amino)phthalazin-1-yl)-3-hydroxybenzonitrile (Compound 111)

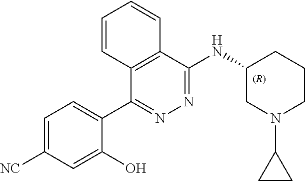

### Step 1: Synthesis of (R)-4-chloro-N-(1-cyclopropylpiperidin-3-yl)phthalazin-1-amine

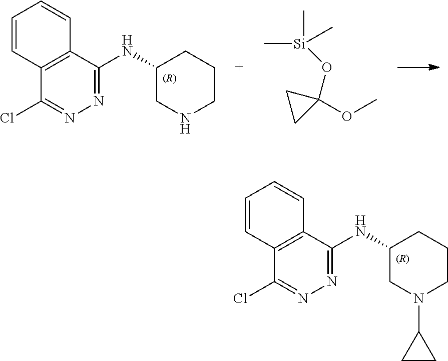

(R)-4-chloro-N-(piperidin-3-yl)phthalazin-1-amine (505.0 mg, 1.92 mmol, 1.0 eq.), (1-methoxycyclopropoxy)trimethylsilane (1.34 g, 7.68 mmol, 4.0 eq.), acetic acid (1.0 mL), cesium fluoride (291.9 mg, 1.92 mmol, 1.5 eq.) and sodium cyanoborohydride (603.8 mg, 9.61 mmol, 5.0 eq.) were added to methanol (15.0 mL), and the mixture was reacted at 60° C. for 12 h. After the reaction was completed as monitored by TLC, a saturated aqueous sodium bicarbonate solution (50.0 mL) was added to the system, followed by extraction with dichloromethane (100.0 mL). The organic phase was dried over anhydrous sodium sulfate and filtered, and the filtrate was concentrated under reduced pressure. The crude product was purified by silica gel column chromatography (dichloromethane:methanol=80:1 to 50:1) to give the product (350.0 mg, yield: 60.1%).

### Step 2: Synthesis of (R)-4-(4-((1-cyclopropylpiperidin-3-yl)amino)phthalazin-1-yl)-3-(ethoxymethoxy)benzonitrile

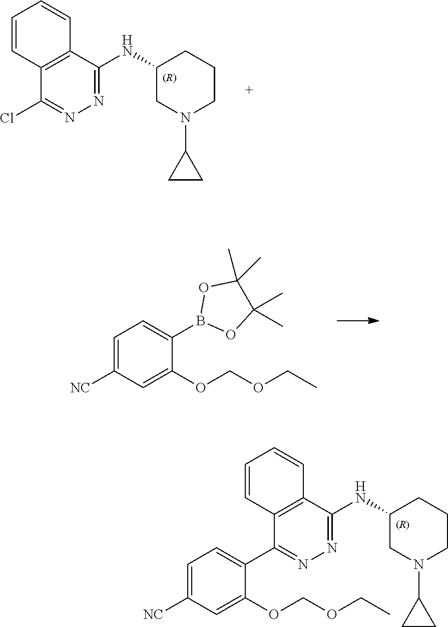

(R)-4-chloro-N-(1-cyclopropylpiperidin-3-yl)phthalazin-1-amine (350.0 mg, 1.15 mmol, 1.0 eq.), 3-(ethoxymethoxy)-4-(4,4,5,5-tetramethyl-1,3,2-dioxaborolan-2-yl)benzonitrile (700.8 mg, 2.31 mmol, 2.0 eq.), sodium bicarbonate (291.0 mg, 3.46 mmol, 3.0 eq.) and Pd(dppf)Cl₂ (42.2 mg, 0.05 mmol, 0.05 eq.) were added to 1,4-dioxane (10.0 mL) and water (5.0 mL), and the mixture was reacted at 110° C. for 5 h in nitrogen atmosphere. After the reaction was completed as monitored by TLC, the system was cooled to room temperature, and water (50.0 mL) was added, followed by extraction with ethyl acetate (100.0 mL). The organic phase was dried over anhydrous sodium sulfate and filtered, and the filtrate was concentrated under reduced pressure. The crude product was purified by silica gel column chromatography (100-200 mesh silica gel, dichloromethane:methanol=80:1 to 60:1) to give the product (300.0 mg, yield: 58.5%).

### Step 3: Synthesis of (R)-4-(4-((1-cyclopropylpiperidin-3-yl)amino)phthalazin-1-yl)-3-hydroxybenzonitrile

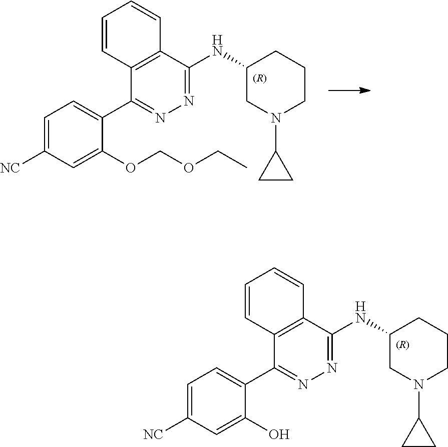

(R)-4-(4-((1-cyclopropylpiperidin-3-yl)amino)phthalazin-1-yl)-3-(ethoxymethoxy)benzonitrile (290.0 mg, 0.65 mmol, 1.0 eq.) was added to ethanol (5.0 mL) and a hydrogen chloride-ethanol solution (2.0 mol/L, 10.0 mL), and the mixture was reacted at room temperature for 1 h. After the reaction was completed as monitored by TLC, the pH of the system was adjusted to 7-8 with a saturated aqueous sodium bicarbonate solution, followed by extraction with dichloromethane (50.0 mL). The organic phase was dried over anhydrous sodium sulfate and filtered, and the filtrate was concentrated under reduced pressure. The crude product was purified by silica gel column chromatography (100-200 mesh silica gel, dichloromethane:methanol=80:1 to 40:1) to give the product (190.0 mg, yield: 75.3%).

¹HNMR (400 MHz, DMSO-d6) δ (ppm): 10.47 (s, 1H), 8.40-8.38 (d, J=8 Hz, 1H), 7.86 (t, J=8 Hz, 1H), 7.78 (t, J=8 Hz, 1H), 7.51-7.49 (d, J=8 Hz, 1H), 7.43 (s, 1H), 7.41-7.33 (m, 2H), 7.16-7.14 (d, J=8 Hz, 1H), 4.36-4.34 (m, 1H), 3.29-3.27 (m, 1H), 2.92-2.89 (m, 1H), 2.22-2.17 (m, 2H), 2.01-1.99 (m, 1H), 1.74-1.66 (m, 2H), 1.57-1.44 (m, 2H), 0.46-0.34 (m, 4H).

Molecular formula: C₂₃H₂₃N₅O Precise molecular weight: 385.19 LC-MS (Pos, m/z)=386.11[M+H]⁺.

### Example 38: Synthesis of (R)-2-(4-((1-methylpiperidin-3-yl)amino)phthalazin-1-yl)-5-(methylthio)phenol (Compound 114)

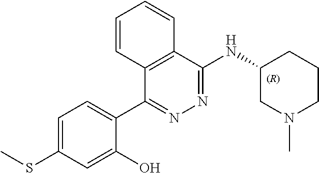

### Step 1: Synthesis of 2-bromo-5-iodophenol

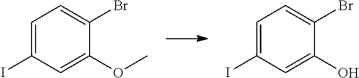

1-Bromo-4-iodo-2-methoxybenzene (6.0 g, 19.17 mmol, 1.0 eq.) was added to dichloromethane (20.0 mL), followed by dropwise and slow addition of boron tribromide (14.4 g, 57.50 mmol, 3.0 eq.). The mixture was reacted at room temperature for 5 h. After the reaction was completed as monitored by TLC, methanol was added to quench the reaction under an ice bath, and the reaction solution was concentrated under reduced pressure, followed by addition of water (50 mL) and extraction with dichloromethane (200.0 mL). The organic phase was dried over anhydrous sodium sulfate and filtered, and the filtrate was concentrated under reduced pressure to give the product (5.3 g, yield: 92.4%).

### Step 2: Synthesis of 1-bromo-2-(ethylmethoxy)-4-iodobenzene

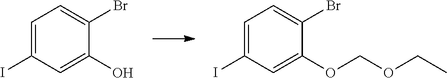

2-Bromo-5-iodophenol (5.3 g, 17.73 mmol, 1.0 eq.) was added into anhydrous tetrahydrofuran (20.0 mL), followed by addition of sodium hydride (60% by mass, 1.0 g, 26.59 mmol, 1.5 eq.) under an ice bath. The mixture was reacted for 0.5 h, and then chloromethyl ethyl ether (2.5 g, 26.59 mmol, 1.5 eq.) was added. The mixture was warmed to room temperature and reacted for 12 h. After the reaction was completed as monitored by TLC, a saturated aqueous ammonium chloride solution (100.0 mL) was added to the system, followed by extraction with ethyl acetate (200.0 mL). The organic phase was dried over anhydrous sodium sulfate and filtered, and the filtrate was concentrated under reduced pressure. The crude product was purified by silica gel column chromatography (100-200 mesh silica gel, petroleum ether:ethyl acetate=100:1) to give the product (6.15 g, yield: 97.3%).

### Step 3: Synthesis of (4-bromo-3-(ethoxymethoxy)phenyl)(methyl)sulfane

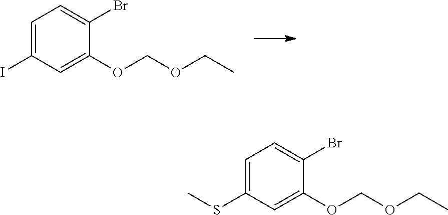

1-Bromo-2-(ethylmethoxy)-4-iodobenzene (6.15 g, 17.22 mmol, 1.0 eq.) was added to anhydrous tetrahydrofuran (20.0 mL), and the mixture was cooled to −30° C., followed by dropwise addition of a solution of an isopropylmagnesium chloride lithium chloride complex in tetrahydrofuran (1.3 mol/L, 15.9 mL, 20.67 mmol, 1.2 eq.). The resulting mixture was reacted for 0.5 h, and then 1,2-dimethyldisulfane (1.78 g, 18.95 mmol, 1.1 eq.) was added. The mixture was warmed to room temperature and stirred for 2 h. After the reaction was completed as monitored by TLC, a saturated aqueous ammonium chloride solution (100.0 mL) was added to the system, followed by extraction with ethyl acetate (200.0 mL). The organic phase was dried over anhydrous sodium sulfate and filtered, and the filtrate was concentrated under reduced pressure to give the product (4.5 g, yield: 94.3%).

### Step 4: Synthesis of 2-(2-(ethoxymethoxy)-4-(methylthio)phenyl)-4,4,5,5-tetramethyl-1,3,2-dioxaborolane

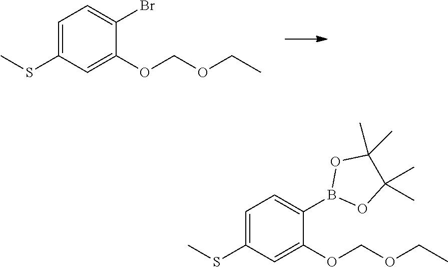 [SMILES: CCOCOc1cc(SC)ccc1Br ; CCOCOc1cc(SC)ccc1B1OC(C)(C)C(C)(C)O1]

(4-Bromo-3-(ethoxymethoxy)phenyl)(methyl)sulfane (4.5 g, 16.23 mmol, 1.0 eq.), 4,4,5,5-tetramethyl-1,3,2-dioxaborolane (6.18 g, 24.35 mmol, 1.5 eq.), potassium acetate (3.18 g, 32.46 mmol, 2.0 eq.) and Pd(dppf)Cl₂ (593.9 mg, 0.81 mmol, 0.05 eq.) were added to 1,4-dioxane (25.0 mL), and the mixture was stirred at 110° C. for 6 h in nitrogen atmosphere. After the reaction was completed as monitored by TLC, water (200.0 mL) was added, followed by extraction with ethyl acetate (200.0 mL). The organic phase was dried over anhydrous sodium sulfate and filtered, and the filtrate was concentrated under reduced pressure. The crude product was purified by silica gel column chromatography (100-200 mesh silica gel, petroleum ether:ethyl acetate=10:1) to give the product (4.8 g, yield: 91.2%).

### Step 5: Synthesis of (R)-4-(2-(ethoxymethoxy)-4-(methylthio)phenyl)-N-(1-methylpiperidin-3-yl)phthalazin-1-amine

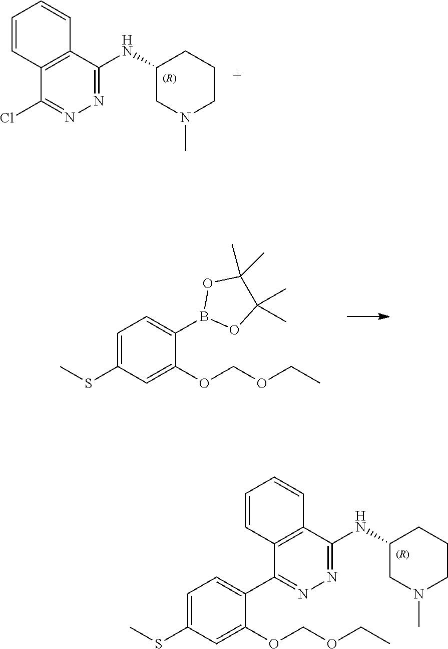

(R)-4-chloro-N-(1-methylpiperidin-3-yl)phthalazin-1-amine (1.0 g, 3.61 mmol, 1.0 eq.), 2-(2-(ethoxymethoxy)-4-(methylthio)phenyl)-4,4,5,5-tetramethyl-1,3,2-dioxaborolane (1.75 g, 5.41 mmol, 1.5 eq.), sodium bicarbonate (607.0 mg, 7.22 mmol, 2.0 eq.) and Pd(dppf)Cl₂ (132.1 mg, 0.18 mmol, 0.05 eq.) were added to a mixed solution of 1,4-dioxane (15.0 mL) and water (7.0 mL), and the mixture was stirred at 110° C. for 6 h in nitrogen atmosphere. After the reaction was completed as monitored by TLC, water (100.0 mL) was added, followed by extraction with ethyl acetate (100.0 mL). The organic phase was dried over anhydrous sodium sulfate and filtered, and the filtrate was concentrated under reduced pressure. The crude product was purified by silica gel column chromatography (100-200 mesh silica gel, dichloromethane:methanol=40:1 to 10:1) to give the product (1.2 g, yield: 75.9%).

### Step 6: Synthesis of (R)-2-(4-((1-methylpiperidin-3-yl)amino)phthalazin-1-yl)-5-(methylthio)phenol

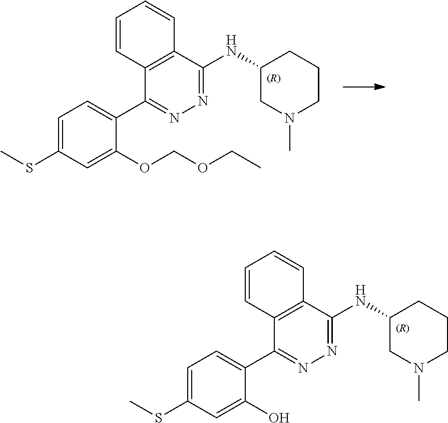

(R)-4-(2-(ethoxymethoxy)-4-(methylthio)phenyl)-N-(1-methylpiperidin-3-yl)phthalazin-1-amine (100.0 mg, 0.22 mmol, 1.0 eq.) and a hydrogen chloride-1,4-dioxane solution (4.0 mol/L, 3.0 mL) were added to dichloromethane (1.0 mL), and the mixture was reacted at room temperature for 2 h. After the reaction was completed as monitored by TLC, the pH of the system was adjusted to 7-8 with a saturated aqueous sodium bicarbonate solution, followed by extraction with dichloromethane (50.0 mL). The organic phase was dried over anhydrous sodium sulfate and filtered, and the filtrate was concentrated under reduced pressure. The crude product was purified by preparative thin-layer chromatography to give the product (60.0 mg, yield: 69.7%).

¹HNMR (400 MHz, DMSO-d6) δ (ppm): 9.92 (s, 1H), 8.38-8.36 (d, J=8 Hz, 1H), 7.84 (t, J=8 Hz, 1H), 7.77 (t, J=8 Hz, 1H), 7.54-7.52 (d, J=8 Hz, 1H), 7.25-7.23 (d, J=8 Hz, 1H), 7.04-7.02 (d, J=8 Hz, 1H), 6.86-6.83 (m, 2H), 4.40 (s, 1H), 3.08-3.06 (d, J=8 Hz, 1H), 2.72-2.69 (d, J=12 Hz, 1H), 2.51 (s, 3H), 2.21 (s, 3H), 1.99-1.88 (m, 3H), 1.77-1.41 (m, 3H).

Molecular formula: C₂₁H₂₄N₄OS Precise molecular weight: 380.17 LC-MS (Pos, m/z)=381.03[M+H]⁺.

### Example 39: Synthesis of (R)-3-hydroxy-4-(4-((tetrahydro-2H-pyran-3-yl)amino)phthalazin-1-yl)benzonitrile (Compound 112)

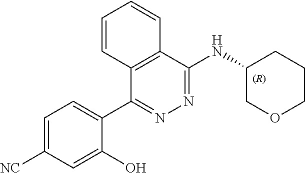

### Step 1: Synthesis of (R)-4-chloro-N-(tetrahydro-2H-pyran-3-yl)phthalazin-1-amine

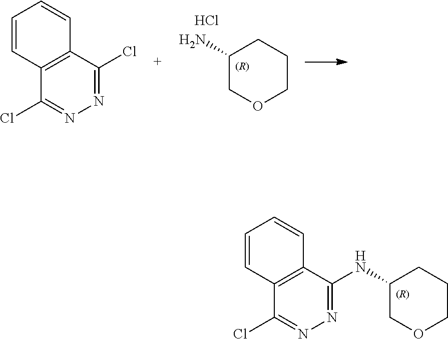

1,4-Dichlorophthalazine (157.0 mg, 0.78 mmol, 1.0 eq.), (R)-tetrahydro-2H-pyran-3-amine hydrochloride (162.8 mg, 1.18 mmol, 1.5 eq.) and diisopropylethylamine (203.6 mg, 1.57 mmol, 2.0 eq.) were added to N,N-dimethylacetamide (3.0 mL), and the mixture was stirred at 120° C. for 12 h. After the reaction was completed as monitored by TLC, the system was cooled to room temperature, and water (100.0 mL) was added, followed by extraction with EA (100.0 mL). The organic phase was dried over anhydrous sodium sulfate and filtered, and the filtrate was concentrated under reduced pressure. The crude product was purified by preparative thin-layer chromatography to give the product (67.0 mg, yield: 32.2%).

### Step 2: Synthesis of (R)-3-(ethoxymethoxy)-4-(4-((tetrahydro-2H-pyran-3-yl)amino)phthalazin-1-yl)benzonitrile

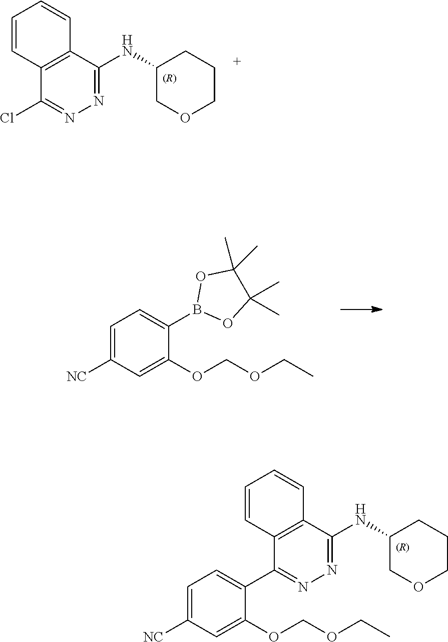

(R)-4-chloro-N-(tetrahydro-2H-pyran-3-yl)phthalazin-1-amine (67.0 mg, 0.25 mmol, 1.0 eq.), 3-(ethoxymethoxy)-4-(4,4,5,5-tetramethyl-1,3,2-dioxaborolan-2-yl)benzonitrile (154.0 mg, 0.50 mmol, 2.0 eq.), sodium bicarbonate (64.0 mg, 0.76 mmol, 3.0 eq.) and Pd(dppf)Cl₂ (9.29 mg, 0.01 mmol, 0.05 eq.) were added to 1,4-dioxane (4.0 mL) and water (2.0 mL), and the mixture was reacted at 110° C. for 5 h in nitrogen atmosphere. After the reaction was completed as monitored by TLC, the system was cooled to room temperature, and water (50.0 mL) was added, followed by extraction with ethyl acetate (100.0 mL). The organic phase was dried over anhydrous sodium sulfate and filtered, and the filtrate was concentrated under reduced pressure. The crude product was purified by preparative thin-layer chromatography to give the product (60.0 mg, yield: 58.4%).

### Step 3: Synthesis of (R)-3-hydroxy-4-(4-((tetrahydro-2H-pyran-3-yl)amino)phthalazin-1-yl)benzonitrile

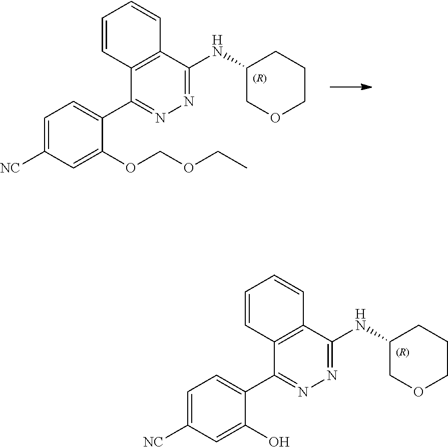

(R)-3-(ethoxymethoxy)-4-(4-((tetrahydro-2H-pyran-3-yl)amino)phthalazin-1-yl)benzonitrile (60.0 mg, 0.14 mmol, 1.0 eq.) and a hydrogen chloride-ethanol solution (2.0 mol/L, 4.0 mL) were added to ethanol (2.0 mL), and the mixture was reacted at room temperature for 2 h. After the reaction was completed as monitored by TLC, the pH of the system was adjusted to 7-8 with saturated sodium bicarbonate, followed by extraction with dichloromethane (50.0 mL). The organic phase was dried over anhydrous sodium sulfate and filtered, and the filtrate was concentrated under reduced pressure to give the product (30.0 mg, yield: 58.5%).

¹HNMR (400 MHz, DMSO-d6) δ (ppm): 10.47 (s, 1H), 8.43-8.40 (d, J=12 Hz, 1H), 7.87 (t, J=8 Hz, 1H), 7.79 (t, J=8 Hz, 1H), 7.51-7.49 (d, J=8 Hz, 1H), 7.43 (s, 1H), 7.41 (s, 1H), 7.33 (s, 1H), 7.23-7.21 (d, J=8 Hz, 1H), 4.44-4.39 (m, 1H), 4.09-4.06 (m, 1H), 3.85-3.82 (d, J=12 Hz, 1H), 3.28-3.16 (m, 2H), 2.11 (s, 1H), 1.79-1.65 (m, 3H).

Molecular formula: C₂₀H₁₈N₄O₂ Precise molecular weight: 346.14 LC-MS (Pos, m/z)=347.02[M+H]⁺.

### Example 40: Synthesis of 4-(4-(((3R,5R)-5-fluoro-1-methylpiperidin-3-yl)amino)phthalazin-1-yl)-3-hydroxybenzonitrile (Compound 123)

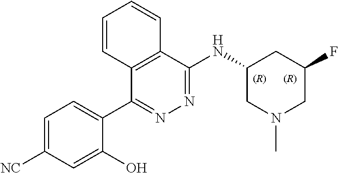

### Step 1: Synthesis of tert-butyl (3R,5R)-3-((4-(4-cyano-2-(ethoxymethoxy)phenyl)phthalazin-1-yl)amino)-5-fluoropiperidine-1-carboxylate

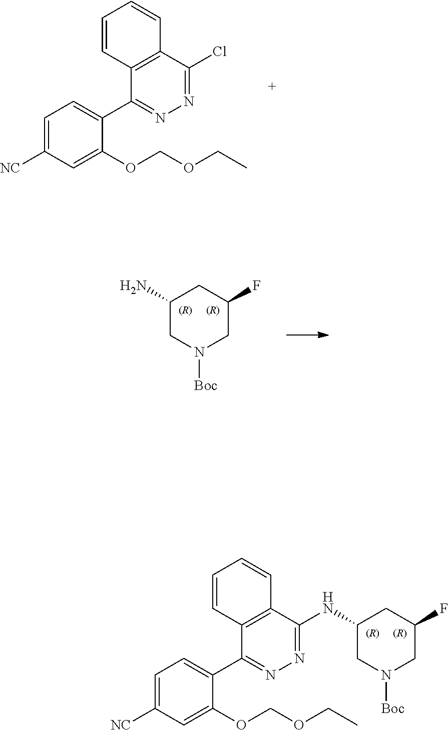

4-(4-Chlorophthalazin-1-yl)-3-(ethoxymethoxy)benzonitrile (292.0 mg, 0.85 mmol, 1.0 eq.), tert-butyl (3R,5R)-3-amino-5-fluoropiperidine-1-carboxylate (206.3 mg, 0.94 mmol, 1.1 eq.), cesium carbonate (559.7 mg, 1.71 mmol, 2.0 eq.), Pd₂(dba)₃ (78.6 mg, 0.08 mmol, 0.1 eq.) and 1,1′-binaphthyl-2,2′-bisdiphenylphosphine (106.9 mg, 0.17 mmol, 0.2 eq.) were added to 1,4-dioxane (15.0 mL), and the mixture was reacted at 90° C. for 12 h in nitrogen atmosphere. After the reaction was completed as monitored by TLC, water (100.0 mL) was added, followed by extraction with ethyl acetate (100.0 mL). The organic phase was dried over anhydrous sodium sulfate and filtered, and the filtrate was concentrated under reduced pressure. The crude product was purified by silica gel column chromatography (100-200 mesh silica gel, dichloromethane:methanol=100:1 to 60:1) to give the product (220.0 mg, yield: 49.1%).

### Step 2: Synthesis of 4-(4-(((3R,5R)-5-fluoropiperidin-3-yl)amino)phthalazin-1-yl)-3-hydroxybenzonitrile

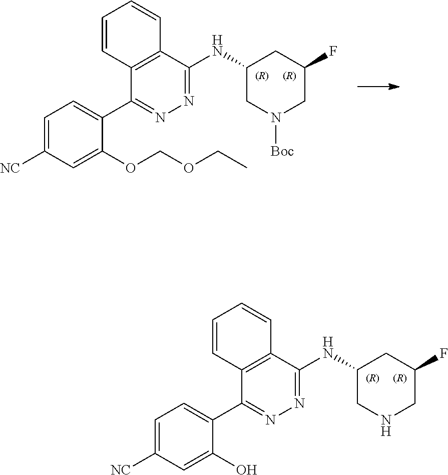

Tert-butyl (3R,5R)-3-((4-(4-cyano-2-(ethoxymethoxy)phenyl)phthalazin-1-yl)amino)-5-fluoropiperidine-1-carboxylate (220.0 mg, 0.42 mmol, 1.0 eq.) and a hydrogen chloride-1,4-dioxane solution (4.0 mol/L, 5.0 mL) were added to dichloromethane (3.0 mL), and the mixture was stirred at room temperature for 1 h. After the reaction was completed as monitored by TLC, the pH of the system was adjusted to 7-8 with a saturated aqueous sodium bicarbonate solution, followed by extraction with dichloromethane (100.0 mL). The organic phase was dried over anhydrous sodium sulfate and filtered, and the filtrate was concentrated under reduced pressure to give the product (150.0 mg, yield: 98.0%).

### Step 3: Synthesis of 4-(4-(((3R,5R)-5-fluoro-1-methylpiperidin-3-yl)amino)phthalazin-1-yl)-3-hydroxybenzonitrile

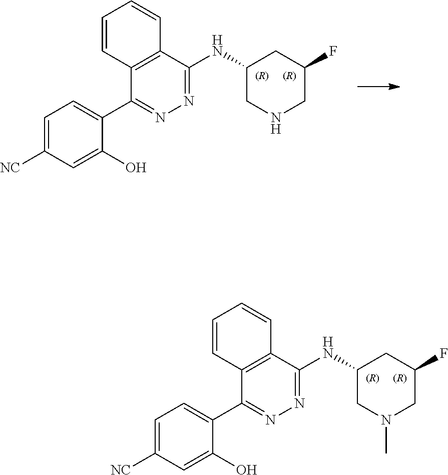

4-(4-(((3R,5R)-5-fluoropiperidin-3-yl)amino)phthalazin-1-yl)-3-hydroxybenzonitrile (150.0 mg, 0.41 mmol, 1.0 eq.) and an aqueous formaldehyde solution (37% by mass, 50.2 mg, 0.61 mmol, 1.5 eq.) were added to methanol (6.0 mL), and the mixture was stirred at room temperature for 0.5 h, followed by addition of sodium cyanoborohydride (38.8 mg, 0.61 mmol, 1.5 eq.). The resulting mixture was reacted at room temperature for 2 h. After the reaction was completed as monitored by TLC, a saturated aqueous sodium bicarbonate solution (50.0 mL) was added, followed by extraction with dichloromethane (100.0 mL). The organic phase was dried over anhydrous sodium sulfate and filtered, and the filtrate was concentrated under reduced pressure. The crude product was purified by preparative thin-layer chromatography to give the product (80.0 mg, yield: 51.6%).

¹HNMR (400 MHz, DMSO-d6) δ (ppm): 10.47 (s, 1H), 8.40-8.38 (d, J=8 Hz, 1H), 7.90-7.86 (m, 1H), 7.81-7.78 (m, 1H), 7.51-7.49 (d, J=8 Hz, 1H), 7.44-7.41 (m, 2H), 7.33 (s, 1H), 7.25-7.23 (d, J=8 Hz, 1H), 5.07-4.96 (m, 1H), 4.75-4.74 (m, 1H), 3.11-3.09 (d, J=8 Hz, 1H), 2.97-2.92 (m, 1H), 2.24 (s, 3H), 2.21 (s, 1H), 2.14-2.11 (m, 1H), 2.02-1.73 (m, 2H).

Molecular formula: C₂₁H₂₀FN₅O Precise molecular weight: 377.17 LC-MS (Pos, m/z)=378.11[M+H]⁺.

### Example 41: Synthesis of (R)-5-chloro-2-(4-((1-methylpiperidin-3-yl)amino)phthalazin-1-yl)phenol (Compound 101)

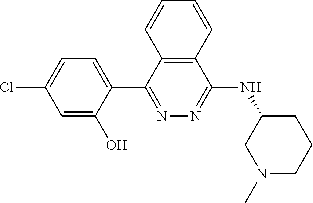 [SMILES: CN1CCC[C@@H](Nc2nnc(-c3ccc(Cl)cc3O)c3ccccc23)C1]

### Step 1: Synthesis of (R)-4-(4-chloro-2-methoxyphenyl)-N-(1-methylpiperidin-3-yl)phthalazin-1-amine

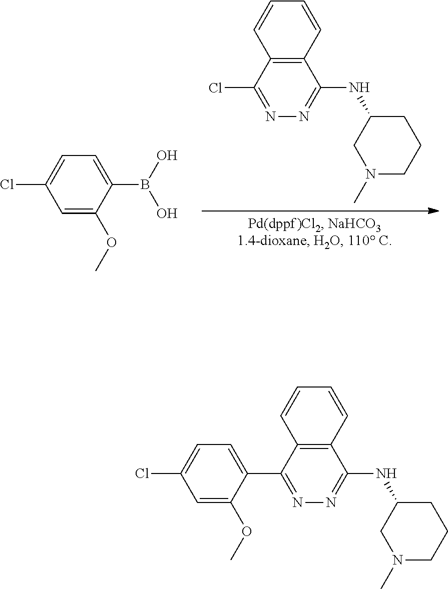 [SMILES: COc1cc(Cl)ccc1B(O)O ; CN1CCC[C@@H](Nc2nnc(Cl)c3ccccc23)C1 ; "Pd(dppf)Cl2, NaHCO31.4-dioxane, H2O, 110° C." ; COc1cc(Cl)ccc1-c1nnc(N[C@@H]2CCCN(C)C2)c2ccccc12]

4-Chloro-2-methoxyphenylboronic acid (2.02 g, 10.8 mmol, 1.0 eq.), (R)-4-chloro-N-(1-methylpiperidin-3-yl)phthalazin-1-amine (3.00 g, 10.8 mmol, 1.0 eq.), Pd(dppf)Cl₂ (790 mg, 1.08 mmol, 0.1 eq.), and NaHCO₃ (1.81 g, 21.6 mmol, 2.0 eq.) were added successively to 1,4-dioxane (30 mL) and water (15 mL), and the mixture was heated to 110° C. and reacted for 2 h in nitrogen atmosphere. The reaction solution was cooled to room temperature, and water (60 mL) was added to quench the reaction, followed by extraction with EA (30 mL×2). The organic phase was dried over anhydrous sodium sulfate and filtered, and the filtrate was concentrated under reduced pressure. The crude product was separated by silica gel column chromatography (DCM:MeOH=50:1 to 10:1) to give the product (3.50 g, yield: 84.3%).

### Step 2: Synthesis of (R)-5-chloro-2-(4-((1-methylpiperidin-3-yl)amino)phthalazin-1-yl)phenol

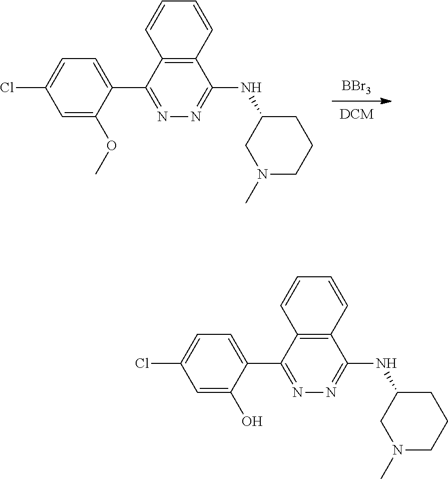

(R)-4-(4-chloro-2-methoxyphenyl)-N-(1-methylpiperidin-3-yl)phthalazin-1-amine (3.50 g, 9.14 mmol, 1.0 eq.) was dissolved in DCM (30 mL), and the mixture was cooled to 0° C., followed by dropwise addition of BBr₃ (4.58 g, 18.3 mmol, 2.0 eq.). The resulting mixture was reacted for 6 h. MeOH was added to quench the reaction, and a saturated aqueous NaHCO₃ solution was added to adjust the pH to 8, followed by extraction with DCM (30 mL×2). The organic phase was dried over anhydrous sodium sulfate and filtered, and the filtrate was concentrated under reduced pressure. The crude product was separated by silica gel column chromatography (DCM:MeOH=50:1 to 10:1) to give the product (1.44 g, yield: 42.7%).

¹HNMR (400 MHz, CDCl₃) δ(ppm): 8.26-8.24 (s, 1H), 8.01 (d, J=8.0 Hz, 1H), 7.91-7.82 (m, 2H), 7.55 (d, J=8.4 Hz, 1H), 7.19 (d, J=2.4 Hz, 1H), 7.00-6.98 (m, 1H), 6.29 (s, 1H), 4.71-4.69 (m, 1H), 2.82 (s, 2H), 2.52 (d, J=9.6 Hz, 1H), 2.35 (s, 3H), 2.18-2.10 (m, 2H), 1.89-1.80 (m, 1H), 1.68-1.64 (m, 2H).

Molecular formula: C₂₀H₂₁ClN₄O Precise molecular weight: 368.14 LC-MS (Pos, m/z)=369.08[M+H]⁺.

### Example 42: Synthesis of (R)-5-cyclopropyl-2-(4-((1-methylpiperidin-3-yl)amino)phthalazin-1-yl)pyridin-3-ol (Compound 110)

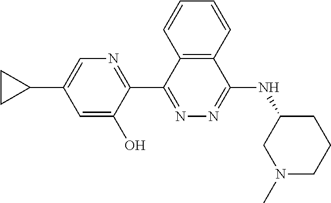 [SMILES: CN1CCC[C@@H](Nc2nnc(-c3ncc(C4CC4)cc3O)c3ccccc23)C1]

### Step 1: Synthesis of 3-bromo-5-(ethoxymethoxy)pyridine

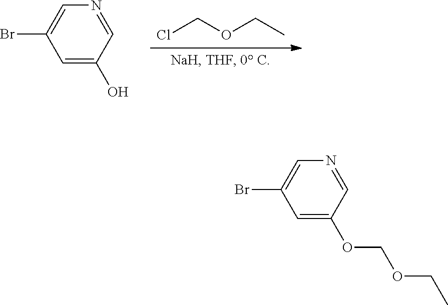

3-Bromo-5-hydroxypyridine (10.0 g, 57.5 mmol, 1.0 eq.) was dissolved in THF (150 mL), and the mixture was cooled to 0° C., followed by portionwise addition of NaH (60% by mass, 3.45 g, 86.2 mmol, 1.5 eq.). The resulting mixture was stirred for 20 min, and chloromethyl ethyl ether (8.15 g, 86.2 mmol, 1.5 eq.) was added. After 1 h of reaction, a saturated aqueous NH₄Cl solution was added to quench the reaction, followed by liquid separation. The aqueous phase was extracted with EA (30 mL×2), and the organic phase was dried over anhydrous sodium sulfate and filtered. The filtrate was concentrated under reduced pressure, and the crude product was separated by silica gel column chromatography (DCM:MeOH=100:1 to 50:1) to give the product (10.8 g, yield: 81.0%).

### Step 2: Synthesis of 3-cyclopropyl-5-(ethoxymethoxy)pyridine

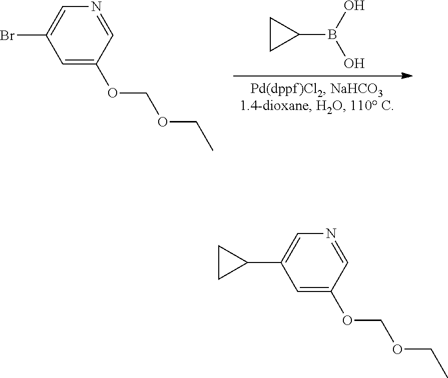

3-Bromo-5-(ethoxymethoxy)pyridine (10.0 g, 43.1 mmol, 1.0 eq.), cyclopropylboronic acid (4.44 g, 51.7 mmol, 1.2 eq.), Pd(dppf)Cl₂ (3.15 g, 4.31 mmol, 0.1 eq.) and NaHCO₃ (7.24 g, 86.2 mmol, 2.0 eq.) were added successively to 1,4-dioxane (100 mL) and water (20 mL), and the mixture was heated to 110° C. and reacted for 6 h in nitrogen atmosphere. The reaction solution was cooled to room temperature, and water (50 mL) was added to quench the reaction, followed by extraction with EA (50 mL×3). The organic phase was dried over anhydrous sodium sulfate and filtered, and the filtrate was concentrated under reduced pressure. The crude product was separated by silica gel column chromatography (DCM:MeOH=100:1 to 50:1) to give the product (6.20 g, yield: 74.5%).

### Step 3: Synthesis of 3-hydroxy-5-cyclopropylpyridine

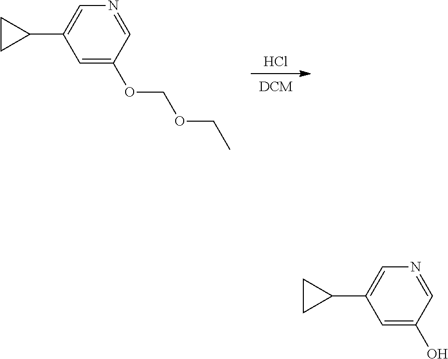

3-Cyclopropyl-5-(ethoxymethoxy)pyridine (6.00 g, 31.0 mmol, 1.0 eq.) was dissolved in DCM (50 mL), followed by dropwise addition of a solution of hydrogen chloride in 1,4-dioxane (4 mol/L, 15.5 mL, 62.0 mmol, 2.0 eq.). The mixture was stirred at room temperature for 6 h. The pH was adjusted to 8 with a saturated aqueous NaHCO₃ solution, followed by extraction with a mixed solvent (DCM:MeOH=10:1) (50 mL×2). The organic phase was dried over anhydrous sodium sulfate and filtered, and the filtrate was concentrated under reduced pressure. The crude product was separated by silica gel column chromatography (DCM:MeOH=100:1 to 20:1) to give the product (3.30 g, yield: 78.6%).

### Step 4: Synthesis of 5-cyclopropyl-2-iodo-3-hydroxypyridine

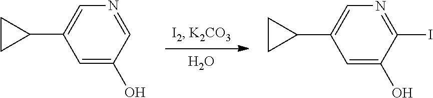

3-Hydroxy-5-cyclopropylpyridine (3.20 g, 23.7 mmol, 1.0 eq.) was added to H₂O (50 mL), followed by addition of K₂CO₃ (6.55 g, 47.4 mmol, 2.0 eq.). The mixture was stirred for dissolution, and then 12 (6.01 g, 23.7 mmol, 1.0 eq.) was added. The mixture was reacted at room temperature for 20 h. The pH was adjusted to 7 with dilute hydrochloric acid, followed by extraction with EA (50 mL×3). The organic phase was dried over anhydrous sodium sulfate and filtered, and the filtrate was concentrated under reduced pressure. The crude product was separated by silica gel column chromatography (DCM:MeOH=100:1 to 20:1) to give the product (4.70 g, yield: 76.0%).

### Step 5: Synthesis of 5-cyclopropyl-3-(ethoxymethoxy)-2-iodopyridine

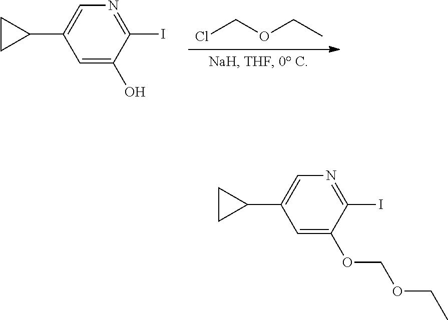

5-Cyclopropyl-2-iodo-3-hydroxypyridine (4.00 g, 15.3 mmol, 1.0 eq.) was dissolved in THF (80 mL), and the mixture was cooled to 0° C., followed by portionwise addition of NaH (60% by mass, 0.920 g, 23.0 mmol, 1.5 eq.). The resulting mixture was stirred for 30 min, and chloromethyl ethyl ether (2.17 g, 23.0 mmol, 1.5 eq.) was added. The mixture was reacted for 4 h. A saturated aqueous NH₄Cl solution was added to quench the reaction, followed by extraction with EA (50 mL×2). The organic phase was dried over anhydrous sodium sulfate and filtered, and the filtrate was concentrated under reduced pressure. The crude product was separated by silica gel column chromatography (PE:EA=20:1 to 5:1) to give the product (4.20 g, yield: 85.9%).

### Step 6: Synthesis of 5-cyclopropyl-3-(ethoxymethoxy)-2-(tributylstannyl)pyridine

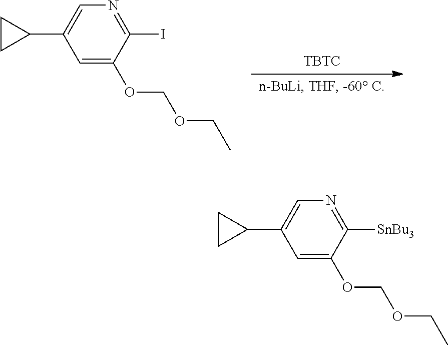

5-Cyclopropyl-3-(ethoxymethoxy)-2-iodopyridine (2.00 g, 6.27 mmol, 1.0 eq.) was dissolved in THF (40 mL), and the mixture was cooled to −60° C. in nitrogen atmosphere, followed by dropwise addition of n-butyllithium (1.6 mol/L tetrahydrofuran solution, 4.7 mL, 7.52 mmol, 1.2 eq.). The mixture was stirred for 1 h, and tributyltin chloride (2.45 g, 7.52 mmol, 1.2 eq.) was added. The mixture was reacted for 1 h. A saturated aqueous potassium fluoride solution was added to quench the reaction, followed by extraction with EA (30 mL×3). The organic phase was dried over anhydrous sodium sulfate and filtered, and the filtrate was concentrated under reduced pressure. The crude product was separated by silica gel column chromatography (PE:EA=10:1) to give the product (2.40 g, yield: 79.4%).

### Step 7: Synthesis of (R)-4-(5-cyclopropyl-3-(ethoxymethoxy)pyridin-2-yl)-N-(1-methylpiperidin-3-yl)phthalazin-1-amine

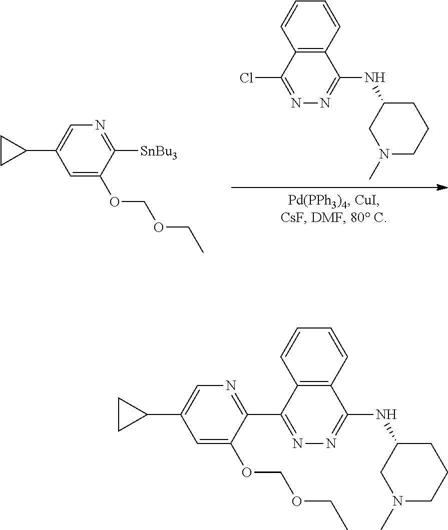

5-Cyclopropyl-3-(ethoxymethoxy)-2-(tributylstannyl)pyridine (1.05 g, 2.17 mmol, 1.5 eq.), (R)-4-chloro-N-(1-methylpiperidin-3-yl)phthalazin-1-amine (400 mg, 1.45 mmol, 1.0 eq.), tetrakis(triphenylphosphine)palladium (167 mg, 0.145 mmol, 0.1 eq.), cuprous iodide (27.5 mg, 0.145 mmol, 0.1 eq.) and cesium fluoride (440 mg, 2.90 mmol, 2.0 eq.) were added successively to DMF (15 mL), and the mixture was heated to 80° C. and reacted for 2 h in nitrogen atmosphere. The reaction solution was cooled to room temperature, and a saturated aqueous potassium fluoride solution was added to quench the reaction, followed by extraction with EA (30 mL×3). The organic phase was dried over anhydrous sodium sulfate and filtered, and the filtrate was concentrated under reduced pressure. The crude product was separated by silica gel column chromatography (DCM:MeOH=20:1 to 10:1) to give the product (530 mg, yield: 84.6%).

### Step 8: Synthesis of (R)-5-cyclopropyl-2-(4-((1-methylpiperidin-3-yl)amino)phthalazin-1-yl)pyridin-3-ol

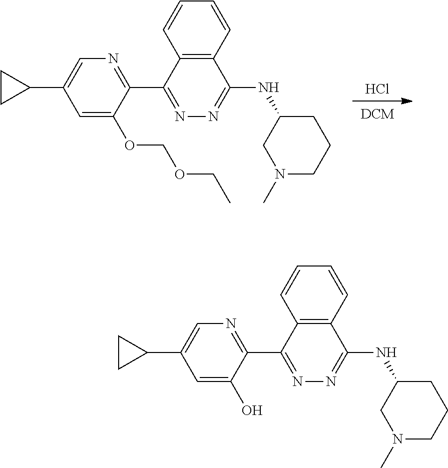

(R)-4-(5-cyclopropyl-3-(ethoxymethoxy)pyridin-2-yl)-N-(1-methylpiperidin-3-yl)phthalazin-1-amine (530 mg, 1.22 mmol, 1.0 eq.) was dissolved in DCM (8 mL), followed by dropwise addition of a solution of hydrogen chloride in 1,4-dioxane (4 mol/L, 1.53 mL, 6.10 mmol, 5.0 eq.). The mixture was stirred at room temperature for 1 h. The pH was adjusted to 9 with a saturated aqueous Na₂CO₃ solution, followed by extraction with DCM (20 mL×3). The organic phase was dried over anhydrous sodium sulfate and filtered, and the filtrate was concentrated under reduced pressure. The crude product was separated by preparative thin-layer chromatography (DCM:MeOH=50:1) to give the product (432 mg, yield: 94.1%).

¹HNMR (400 MHz, DMSO-d6) δ (ppm): 14.55 (s, 1H), 9.73-9.71 (m, 1H), 8.60 (s, 1H), 8.11 (d, J=2.0 Hz, 1H), 7.97-7.94 (m, 2H), 7.75 (s, 1H), 7.01 (d, J=2.0 Hz, 1H), 4.59 (s, 1H), 3.31 (d, J=8.8 Hz, 1H), 2.96 (d, J=11.2 Hz, 1H), 2.48 (s, 4H), 2.00-1.97 (m, 2H), 1.87 (s, 1H), 1.75-1.69 (m, 2H), 1.06-1.03 (m, 2H), 0.84-0.82 (m, 1H).

Molecular formula: C₂₂H₂₅N₅O Precise molecular weight: 375.21 LC-MS (Pos, m/z)=376.11[M+H]⁺.

### Example 43: Synthesis of (R)-3-hydroxy-4-(4-((1-(tetrahydro-2H-pyran-4-yl)piperidin-3-yl)amino)phthalazin-1-yl)benzonitrile (Compound 124)

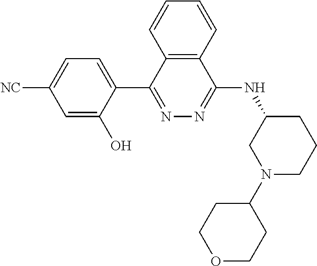 [SMILES: N#Cc1ccc(-c2nnc(N[C@@H]3CCCN(C4CCOCC4)C3)c3ccccc23)c(O)c1]

### Step 1: Synthesis of (R)-4-chloro-N-(1-(tetrahydro-2H-pyran-4-yl)piperidin-3-yl)phthalazin-1-amine

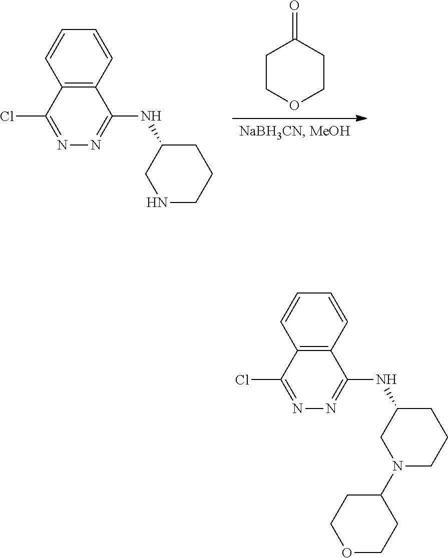

(R)-4-chloro-N-(piperidin-3-yl)phthalazin-1-amine (300 mg, 1.14 mmol, 1.0 eq.) and tetrahydro-4H-pyran-4-one (171 mg, 1.71 mmol, 1.5 eq.) were dissolved in MeOH (5 mL), and the mixture was stirred at room temperature for 1 h. NaBH₃CN (107 mg, 3.36 mmol, 1.5 eq.) was added, and the resulting mixture was reacted for 25 h. A saturated aqueous NaHCO₃ solution was added to quench the reaction, followed by extraction with DCM (20 mL×2). The organic phase was dried over anhydrous sodium sulfate and filtered, and the filtrate was concentrated under reduced pressure. The crude product was separated by silica gel column chromatography (DCM:MeOH=50:1 to 20:1) to give the product (312 mg, yield: 78.8%).

### Step 2: Synthesis of (R)-3-(ethoxymethoxy)-4-(4-((1-(tetrahydro-2H-pyran-4-yl)piperidin-3-yl)amino)phthalazin-1-yl)benzonitrile

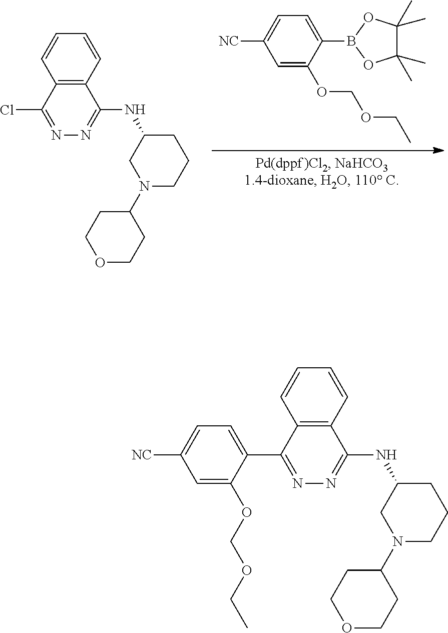 [SMILES: Clc1nnc(N[C@@H]2CCCN(C3CCOCC3)C2)c2ccccc12 ; CCOCOc1cc(C#N)ccc1B1OC(C)(C)C(C)(C)O1 ; "Pd(dppf)Cl2, NaHCO31.4-dioxane, H2O, 110° C." ; CCOCOc1cc(C#N)ccc1-c1nnc(N[C@@H]2CCCN(C3CCOCC3)C2)c2ccccc12]

3-(Ethoxymethoxy)-4-(4,4,5,5-tetramethyl-1,3,2-dioxaborolan-2-yl)benzonitrile (324 mg, 1.07 mmol, 1.2 eq.), (R)-4-chloro-N-(1-(tetrahydro-2H-pyran-4-yl)piperidin-3-yl)phthalazin-1-amine (310 mg, 0.894 mmol, 1.0 eq.), Pd(dppf)Cl₂ (65.4 mg, 0.0894 mmol, 0.1 eq.) and NaHCO₃ (150 mg, 1.79 mmol, 2.0 eq.) were added successively to 1,4-dioxane (6 mL) and water (3 mL), and the mixture was heated to 110° C. and reacted for 2 h in nitrogen atmosphere. The reaction solution was cooled to room temperature, and water (30 mL) was added to quench the reaction, followed by extraction with EA (30 mL×2). The organic phase was dried over anhydrous sodium sulfate and filtered, and the filtrate was concentrated under reduced pressure. The crude product was separated by silica gel column chromatography (DCM:MeOH=50:1 to 20:1) to give the product (290 mg, yield: 67.7%).

### Step 3: Synthesis of (R)-3-hydroxy-4-(4-((1-(tetrahydro-2H-pyran-4-yl)piperidin-3-yl)amino)phthalazin-1-yl)benzonitrile

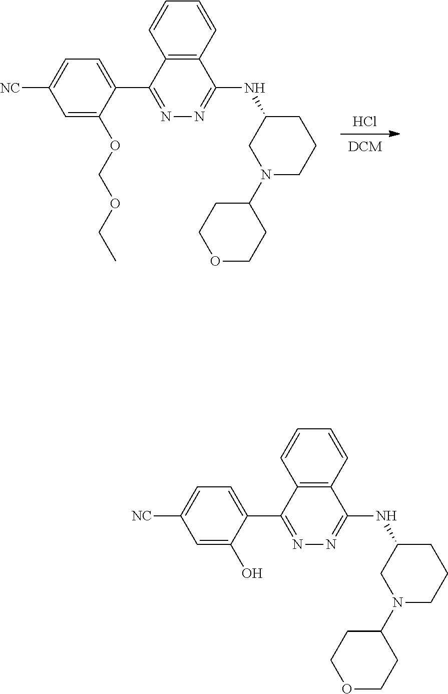

(R)-3-(ethoxymethoxy)-4-(4-((1-(tetrahydro-2H-pyran-4-yl)piperidin-3-yl)amino)phthalazin-1-yl)benzonitrile (290 mg, 0.595 mmol, 1.0 eq.) was dissolved in DCM (3 mL), followed by dropwise addition of a solution of hydrogen chloride in 1,4-dioxane (4 mol/L, 0.75 mL, 2.98 mmol, 5.0 eq.). The mixture was stirred at room temperature for 2 h. The pH was adjusted to 9 with a saturated aqueous Na₂CO₃ solution, followed by extraction with DCM (20 mL×3). The organic phase was dried over anhydrous sodium sulfate and filtered, and the filtrate was concentrated under reduced pressure. The crude product was slurried with a mixed solvent (PE:EA=10:1) to give the product (165 mg, yield: 64.6%).

¹HNMR (400 MHz, DMSO-d6) δ (ppm): 10.61 (s, 1H), 8.39 (s, 1H), 7.86 (s, 1H), 7.79 (s, 1H), 7.49 (d, J=6.8 Hz, 1H), 7.41 (s, 2H), 7.31 (s, 1H), 7.15 (s, 1H), 4.39 (s, 1H), 3.88 (d, J=9.2 Hz, 2H), 3.30-3.24 (m, 4H), 2.84 (d, J=8.0 Hz, 1H), 2.17 (d, J=6.8 Hz, 2H), 2.01 (s, 1H), 1.75 (s, 1H), 1.66 (s, 2H), 1.52-1.49 (m, 4H).

Molecular formula: C₂₅H₂₇N₅O₂ Precise molecular weight: 429.22 LC-MS (Pos, m/z)=430.17[M+H]⁺.

### Example 44: Synthesis of (R)-3-hydroxy-4-(4-((1-isopropylpiperidin-3-yl)amino)phthalazin-1-yl)benzonitrile (Compound 125)

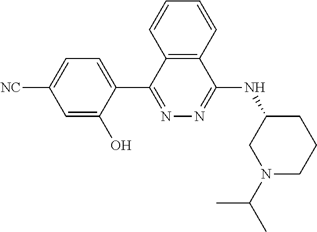 [SMILES: CC(C)N1CCC[C@@H](Nc2nnc(-c3ccc(C#N)cc3O)c3ccccc23)C1]

### Step 1: Synthesis of (R)-4-chloro-N-(1-isopropylpiperidin-3-yl)phthalazin-1-amine

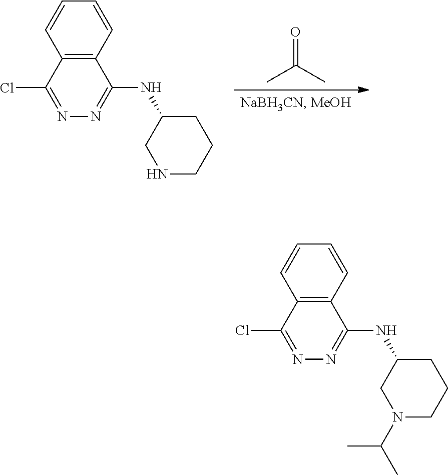

(R)-4-chloro-N-(piperidin-3-yl)phthalazin-1-amine (300 mg, 1.14 mmol, 1.0 eq.) was dissolved in MeOH (5 mL), followed by addition of acetone (99.3 mg, 1.71 mmol, 1.5 eq.). The mixture was stirred at room temperature for 2 h. NaBH₃CN (107 mg, 3.36 mmol, 1.5 eq.) was added, and the resulting mixture was reacted for 25 h. A saturated aqueous NaHCO₃ solution was added to quench the reaction, followed by extraction with DCM (20 mL×2). The organic phase was dried over anhydrous sodium sulfate and filtered, and the filtrate was concentrated under reduced pressure. The crude product was separated by silica gel column chromatography (DCM:MeOH=50:1 to 20:1) to give the product (280 mg, yield: 80.4%).

### Step 2: Synthesis of (R)-3-(ethoxymethoxy)-4-(4-((1-isopropylpiperidin-3-yl)amino)phthalazin-1-yl)benzonitrile

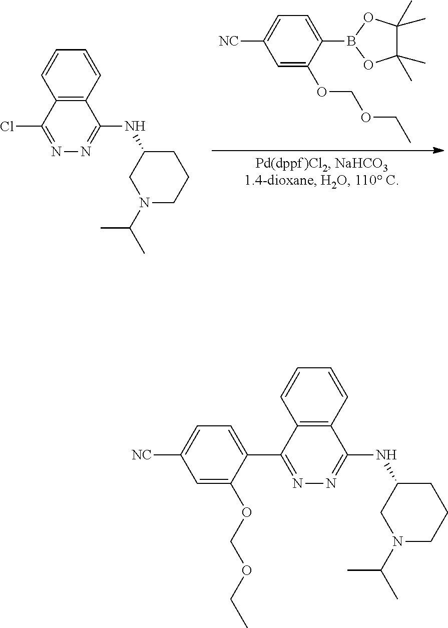 [SMILES: CC(C)N1CCC[C@@H](Nc2nnc(Cl)c3ccccc23)C1 ; CCOCOc1cc(C#N)ccc1B1OC(C)(C)C(C)(C)O1 ; "Pd(dppf)Cl2, NaHCO31.4-dioxane, H2O, 110° C." ; CCOCOc1cc(C#N)ccc1-c1nnc(N[C@@H]2CCCN(C(C)C)C2)c2ccccc12]

3-(Ethoxymethoxy)-4-(4,4,5,5-tetramethyl-1,3,2-dioxaborolan-2-yl)benzonitrile (334 mg, 1.10 mmol, 1.2 eq.), (R)-4-chloro-N-(1-isopropylpiperidin-3-yl)phthalazin-1-amine (280 mg, 0.919 mmol, 1.0 eq.), Pd(dppf)Cl₂ (67.2 mg, 0.0917 mmol, 0.1 eq.) and NaHCO₃ (155 mg, 1.84 mmol, 2.0 eq.) were added successively to 1,4-dioxane (6 mL) and water (3 mL), and the mixture was heated to 110° C. and reacted for 2 h in nitrogen atmosphere. The reaction solution was cooled to room temperature, and water (30 mL) was added to quench the reaction, followed by extraction with EA (30 mL×2). The organic phase was dried over anhydrous sodium sulfate and filtered, and the filtrate was concentrated under reduced pressure. The crude product was separated by silica gel column chromatography (DCM:MeOH=50:1 to 10:1) to give the product (295 mg, yield: 65.5%).

### Step 3: Synthesis of (R)-3-hydroxy-4-(4-((1-isopropylpiperidin-3-yl)amino)phthalazin-1-yl)benzonitrile

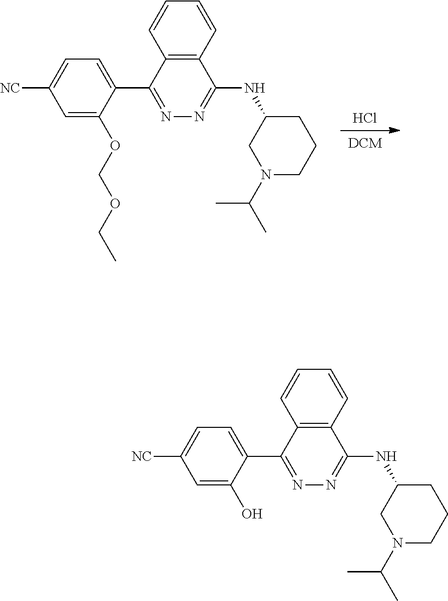

(R)-3-(ethoxymethoxy)-4-(4-((1-isopropylpiperidin-3-yl)amino)phthalazin-1-yl)benzonitrile (295 mg, 0.662 mmol, 1.0 eq.) was dissolved in DCM (3 mL), followed by dropwise addition of a solution of hydrogen chloride in 1,4-dioxane (4 mol/L, 0.83 mL, 3.31 mmol, 5.0 eq.). The mixture was stirred at room temperature for 2 h. The pH was adjusted to 9 with a saturated aqueous Na₂CO₃ solution, followed by extraction with DCM (20 mL×3). The organic phase was dried over anhydrous sodium sulfate and filtered, and the filtrate was concentrated under reduced pressure. The crude product was separated by preparative thin-layer chromatography (DCM:MeOH=10:1) to give the product (152 mg, yield: 59.2%).

¹HNMR (400 MHz, DMSO-d6) δ (ppm): 10.51 (s, 1H), 8.39 (d, J=8.4 Hz, 1H), 7.88-7.84 (m, 1H), 7.80-7.77 (m, 1H), 7.50 (d, J=7.6 Hz, 1H), 7.42 (d, J=7.6 Hz, 2H), 7.32 (d, J=1.2 Hz, 1H), 7.15 (d, J=8.0 Hz, 1H), 4.41-4.39 (m, 1H), 3.11 (d, J=8.0 Hz, 1H), 2.76 (s, 2H), 2.17-2.15 (m, 2H), 2.01-2.99 (m, 1H), 1.77-1.74 (m, 1H), 1.57-1.46 (m, 2H), 1.01-0.98 (m, 6H).

Molecular formula: C₂₃H₂₅N₅O Precise molecular weight: 387.21 LC-MS (Pos, m/z)=388.14[M+H]⁺.

### Example 45: Synthesis of (R)-6-methyl-3-(4-((1-methylpiperidin-3-yl)amino)phthalazin-1-yl)pyridin-2-ol (Compound 106)

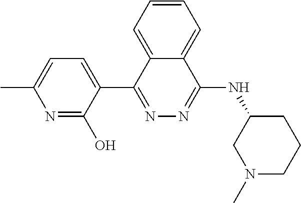 [SMILES: Cc1ccc(-c2nnc(N[C@@H]3CCCN(C)C3)c3ccccc23)c(O)n1]

### Step 1: Synthesis of (2-methoxy-6-methylpyridin-3-yl)boronic acid

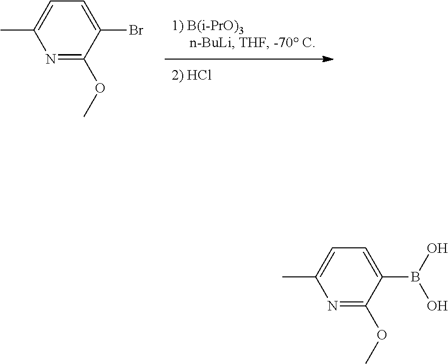

3-Bromo-2-methoxy-6-methylpyridine (6.00 g, 29.7 mmol, 1.0 eq.) and triisopropyl borate (11.3 g, 59.4 mmol, 2.0 eq.) were dissolved in THF (60 mL), followed by purge with nitrogen for protection. The mixture was cooled to −70° C., followed by dropwise addition of n-butyllithium (23.8 mL, 59.4 mmol, 2.0 eq.). The mixture was reacted for 2 h. Dilute hydrochloric acid was added to quench the reaction, and the pH was adjusted to 3. The mixture was stirred at room temperature for 30 min and then extracted with a mixed solvent of DCM:MeOH (10:1) (50 mL×5). The organic phase was dried over anhydrous sodium sulfate and filtered, and the filtrate was concentrated under reduced pressure and slurried with petroleum ether to give (2-methoxy-6-methylpyridin-3-yl)boronic acid (4.20 g, yield: 84.7%).

### Step 2: Synthesis of 1-chloro-4-(2-methoxy-6-methylpyridin-3-yl)phthalazine

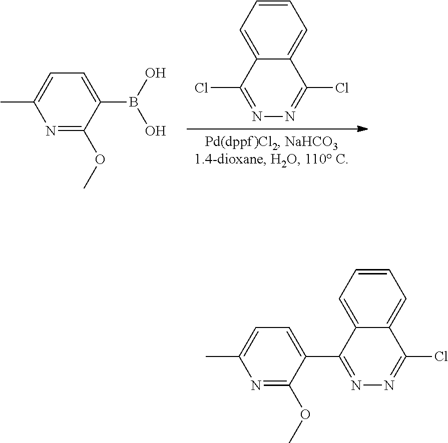

(2-Methoxy-6-methylpyridin-3-yl)boronic acid (3.0 g, 18.0 mmol, 1.0 eq.), 1,4-dichlorophthalazine (4.30 g, 21.6 mmol, 1.2 eq.), Pd(dppf)Cl₂ (659 mg, 0.900 mmol, 0.05 eq.) and NaHCO₃ (3.02 g, 36.0 mmol, 2.0 eq.) were added successively to 1,4-dioxane (30 mL), and H₂O (15 mL) was added, followed by purge with nitrogen for protection. The oil bath was heated to 110° C. and the mixture was reacted for 2 h. The reaction solution was cooled down, and water was added to quench the reaction, followed by extraction with EA (40 mL). The organic phase was dried over anhydrous sodium sulfate and filtered, and the filtrate was concentrated under reduced pressure. The crude product was purified by silica gel column chromatography (PE:EA=10:1) to give the product (2.20 g, yield: 42.9%).

### Step 3: Synthesis of tert-butyl (R)-3-((4-(2-methoxy-6-methylpyridin-3-yl)phthalazin-1-yl)amino)piperidine-1-carboxylate

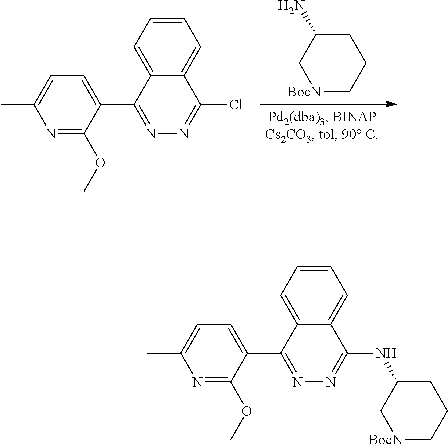

1-Chloro-4-(2-methoxy-6-methylpyridin-3-yl)phthalazine (2.00 g, 7.00 mmol, 1.0 eq.), tert-butyl (R)-3-aminopiperidine-1-carboxylate (1.54 g, 7.70 mmol, 1.1 eq.), Pd₂(dba)₃ (641 mg, 0.700 mmol, 0.1 eq.), BINAP (871 mg, 1.40 mmol, 0.2 eq.) and Cs₂CO₃ (4.56 g, 14.0 mmol, 2.0 eq.) were added successively to toluene (40 mL), followed by purge with nitrogen for protection. The oil bath was heated to 90° C. and the mixture was reacted for 20 h. The reaction solution was cooled down, and water was added to quench the reaction, followed by extraction with EA. The organic phase was dried over anhydrous sodium sulfate and filtered, and the filtrate was concentrated under reduced pressure. The crude product was purified by silica gel column chromatography (DCM:MeOH=100:1 to 40:1) to give the product (1.50 g, yield: 47.7%).

### Step 4: Synthesis of (R)-6-methyl-3-(4-(piperidin-3-ylamino)phthalazin-1-yl)pyridin-2-ol

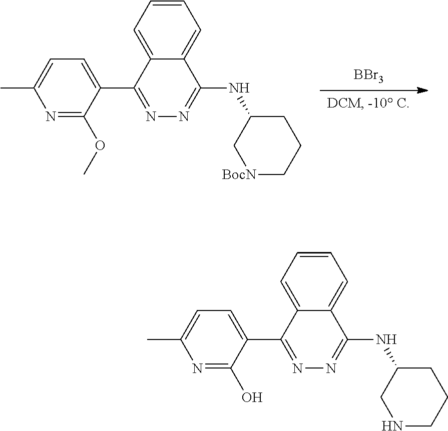 [SMILES: COc1nc(C)ccc1-c1nnc(N[C@@H]2CCCN(C(=O)OC(C)(C)C)C2)c2ccccc12 ; "BBr3" ; "DCM, -10° C." ; Cc1ccc(-c2nnc(N[C@@H]3CCCNC3)c3ccccc23)c(O)n1]

Tert-butyl (R)-3-((4-(2-methoxy-6-methylpyridin-3-yl)phthalazin-1-yl)amino)piperidine-1-carboxylate (1.00 g, 2.22 mmol, 1.0 eq.) was dissolved in DCM (20 mL), and the mixture was cooled to −10° C., followed by dropwise addition of BBr₃ (2.78 g, 11.1 mmol, 5.0 eq.). The mixture was reacted for 4 h. MeOH was added to quench the reaction, and the reaction solution was concentrated. The crude product was dissolved in H₂O, followed by extraction with EA. A saturated aqueous NaHCO₃ solution was added to the aqueous phase to adjust the pH to 8, and DCM (20 mL×3) was added for extraction. The organic phase was dried over anhydrous sodium sulfate and filtered, and the filtrate was concentrated under reduced pressure to give the product (480 mg, yield: 64.3%)

### Step 5: Synthesis of (R)-6-methyl-3-(4-((1-methylpiperidin-3-yl)amino)phthalazin-1-yl)pyridin-2-ol

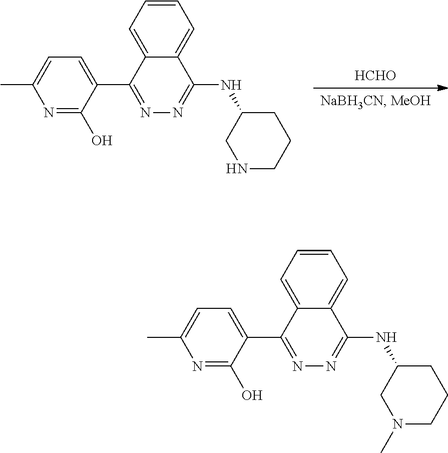

(R)-6-methyl-3-(4-(piperidin-3-ylamino)phthalazin-1-yl)pyridin-2-ol (400 mg, 1.19 mmol, 1.0 eq.) was dissolved in MeOH (10 mL), followed by addition of an aqueous formaldehyde solution (37%, 143 mg, 3.57 mmol, 3.0 eq.). The mixture was stirred at room temperature for 30 min. NaBH₃CN (224 mg, 3.57 mmol, 3.0 eq.) was then added and the mixture was reacted for 2 h. Water was added to quench the reaction, followed by extraction with EA (30 mL×3). The aqueous phase was concentrated under reduced pressure, and the crude product was separated by reversed phase preparative column chromatography (acetonitrile: 0.1% ammonia solution=4:1) to give the product (180 mg, yield: 43.2%).

¹HNMR (300 MHz, DMSO-d6) δ (ppm): 11.95 (s, 1H), 8.50 (s, 1H), 7.89-7.77 (m, 2H), 7.60-7.52 (m, 3H), 6.19 (d, J=6.9 Hz, 1H), 4.74 (s, 1H), 3.63-3.59 (m, 2H), 3.17 (s, 1H), 2.78 (s, 4H), 2.30 (s, 3H), 2.08 (s, 1H), 1.84 (s, 3H).

Molecular formula: C₂₀H₂₃N₅O Molecular weight: 349.44 LC-MS (Pos, m/z)=350.28[M+H]⁺.

### Example 46: Synthesis of 3-hydroxy-4-(4-(((cis)-3-hydroxy-3-methylcyclobutyl)amino)phthalazin-1-yl)benzonitrile (Compound 116)

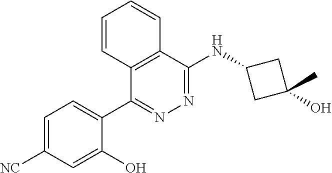 [SMILES: C[C@]1(O)C[C@@H](Nc2nnc(-c3ccc(C#N)cc3O)c3ccccc23)C1]

### Step 1: Synthesis of 4-(4-chlorophthalazin-1-yl)-3-(ethoxymethoxy)benzonitrile

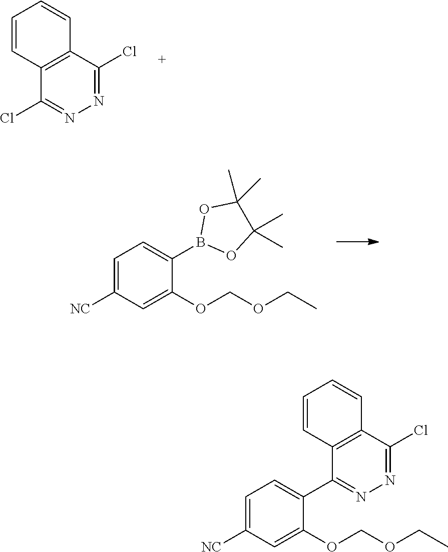 [SMILES: Clc1nnc(Cl)c2ccccc12 ; CCOCOc1cc(C#N)ccc1B1OC(C)(C)C(C)(C)O1 ; CCOCOc1cc(C#N)ccc1-c1nnc(Cl)c2ccccc12]

1,4-Dichlorophthalazine (1.0 g, 5.02 mmol, 1.0 eq.), 3-(ethoxymethoxy)-4-(4,4,5,5-tetramethyl-1,3,2-dioxaborolan-2-yl)benzonitrile (1.82 g, 6.02 mmol, 1.2 eq.), sodium bicarbonate (844.1 mg, 10.04 mmol, 2.0 eq.) and Pd(dppf)Cl₂ (183.8 mg, 0.25 mmol, 0.05 eq.) were added to a mixed solution of 1,4-dioxane (15.0 mL) and water (7.0 mL), and the mixture was reacted at 95° C. for 12 h in nitrogen atmosphere. After the reaction was completed as monitored by TLC, water (100.0 mL) was added, followed by extraction with ethyl acetate (100.0 mL). The organic phase was dried over anhydrous sodium sulfate and filtered, and the filtrate was concentrated under reduced pressure. The crude product was purified by silica gel column chromatography (petroleum ether:ethyl acetate=20:1 to 2:1) to give the product (680.4 mg, yield: 40.0%).

### Step 2: Synthesis of 3-(ethoxymethoxy)-4-(4-(((cis)-3-hydroxy-3-methylcyclobutyl)amino)phthalazin-1-yl)benzonitrile

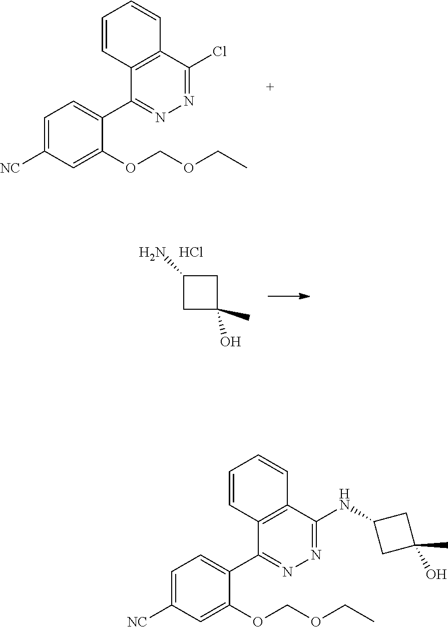

4-(4-Chlorophthalazin-1-yl)-3-(ethoxymethoxy)benzonitrile (200.0 mg, 0.58 mmol, 1.0 eq.), (cis)-3-amino-1-methylcyclobutan-1-ol hydrochloride (161.9 mg, 1.17 mmol, 2.0 eq.) and N,N-diisopropylethylamine (303.9 mg, 2.35 mmol, 4.0 eq.) were added to N,N-dimethylacetamide (6.0 mL), and the mixture was reacted at 110° C. for 5 h. After the reaction was completed as monitored by TLC, water (50.0 mL) was added, followed by extraction with ethyl acetate (100.0 mL). The organic phase was dried over anhydrous sodium sulfate and filtered, and the filtrate was concentrated under reduced pressure to give the crude product (315.0 mg, used directly in the next step).

### Step 3: Synthesis of 3-hydroxy-4-(4-(((cis)-3-hydroxy-3-methylcyclobutyl)amino)phthalazin-1-yl)benzonitrile

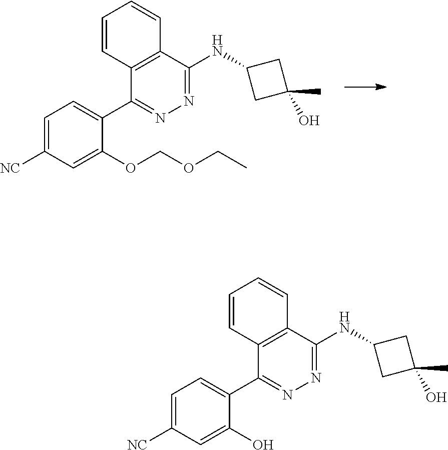 [SMILES: CCOCOc1cc(C#N)ccc1-c1nnc(N[C@H]2C[C@@](C)(O)C2)c2ccccc12 ; C[C@]1(O)C[C@@H](Nc2nnc(-c3ccc(C#N)cc3O)c3ccccc23)C1]

3-(Ethoxymethoxy)-4-(4-(((cis)-3-hydroxy-3-methylcyclobutyl)amino)phthalazin-1-yl)benzonitrile (315.0 mg crude product, 0.58 mmol, 1.0 eq.) was added to dichloromethane (2.0 mL), followed by dropwise addition of a hydrogen chloride-1,4-dioxane solution (4.0 mol/L, 3.0 mL). The mixture was reacted at room temperature for 2 h. After the reaction was completed as monitored by TLC, the pH of the system was adjusted to 7-8 with a saturated aqueous sodium bicarbonate solution, followed by extraction with dichloromethane (100.0 mL). The organic phase was dried over anhydrous sodium sulfate and filtered, and the filtrate was concentrated under reduced pressure. The crude product was purified by preparative thin-layer chromatography (dichloromethane:methanol=10:1) to give the product (20.0 mg, yield: 9.8%).

¹HNMR (400 MHz, DMSO-d6) δ (ppm): 10.47 (s, 1H), 8.44-8.42 (d, J=8 Hz, 1H), 7.86 (t, J=8 Hz, 1H), 7.78 (t, J=8 Hz, 1H), 7.67-7.65 (d, J=8 Hz, 1H), 7.50-7.48 (d, J=8 Hz, 1H), 7.42-7.40 (d, J=8 Hz, 2H), 7.33-7.32 (d, J=4 Hz, 1H), 5.02 (s, 1H), 4.27-4.22 (m, 1H), 2.50-2.46 (m, 2H), 2.20-2.15 (m, 2H), 1.34 (s, 3H).

Molecular formula: C₂₀H₁₈N₄O₂ Precise molecular weight: 346.14 LC-MS (m/z)=347.10[M+H]⁺.

### Example 47: Synthesis of (R)-3-methyl-4-(4-((1-methylpiperidin-3-yl)amino)phthalazin-1-yl)benzonitrile (Compound 118)

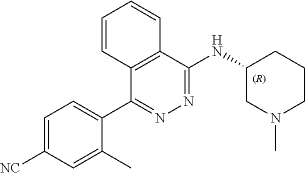

### Step 1: Synthesis of 3-methyl-4-(4,4,5,5-tetramethyl-1,3,2-dioxaborolan-2-yl)benzonitrile

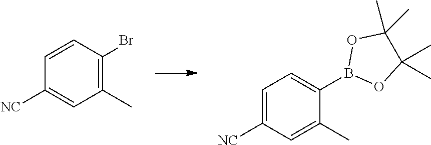

4-Bromo-3-methylbenzonitrile (1.0 g, 5.10 mmol, 1.0 eq.), 4,4,5,5-tetramethyl-1,3,2-dioxaborolane (1.94 g, 7.65 mmol, 1.5 eq.), potassium acetate (1.0 g, 10.20 mmol, 2.0 eq.) and Pd(dppf)Cl₂ (186.5 mg, 0.25 mmol, 0.05 eq.) were added to 1,4-dioxane (20.0 mL), and the mixture was reacted at 100° C. for 4 h in nitrogen atmosphere. After the reaction was completed as monitored by TLC, water (100.0 mL) was added, followed by extraction with ethyl acetate (100.0 mL). The organic phase was dried over anhydrous sodium sulfate and filtered, and the filtrate was concentrated under reduced pressure. The crude product was purified by silica gel column chromatography (specification of silica gel: 100-200 mesh, petroleum ether:ethyl acetate=10:1) to give the product (1.0 g, yield: 80.7%).

### Step 2: Synthesis of (R)-3-methyl-4-(4-((1-methylpiperidin-3-yl)amino)phthalazin-1-yl)benzonitrile

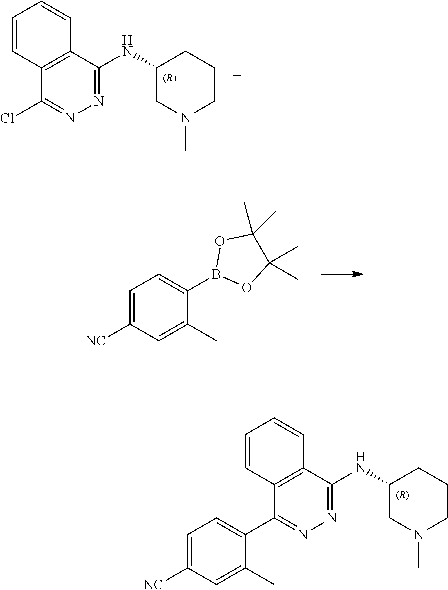

(R)-4-chloro-N-(1-methylpiperidin-3-yl)phthalazin-1-amine (100.0 mg, 0.36 mmol, 1.0 eq.), 3-methyl-4-(4,4,5,5-tetramethyl-1,3,2-dioxaborolan-2-yl)benzonitrile (131.7 mg, 0.54 mmol, 1.5 eq.), sodium bicarbonate (60.6 mg, 0.72 mmol, 2.0 eq.) and Pd(dppf)Cl₂ (13.2 mg, 0.01 mmol, 0.05 eq.) were added to a mixed solution of 1,4-dioxane (10.0 mL) and water (5.0 mL), and the mixture was reacted at 110° C. for 3 h in nitrogen atmosphere. After the reaction was completed as monitored by TLC, water (100.0 mL) was added, followed by extraction with ethyl acetate (100.0 mL). The organic phase was dried over anhydrous sodium sulfate and filtered, and the filtrate was concentrated under reduced pressure. The crude product was purified by preparative thin-layer chromatography (dichloromethane:methanol=10:1) to give the product (40.0 mg, yield: 31.0%).

¹HNMR (400 MHz, DMSO-d6) δ (ppm): 8.47-8.45 (d, J=8 Hz, 1H), 7.91-7.87 (m, 2H), 7.83-7.78 (m, 2H), 7.51-7.49 (d, J=8 Hz, 1H), 7.26-7.25 (d, J=4 Hz, 1H), 7.21-7.19 (d, J=8 Hz, 1H), 4.44-4.42 (m, 1H), 3.09-3.07 (d, J=8 Hz, 1H), 2.72-2.70 (d, J=8 Hz, 1H), 2.21 (s, 3H), 2.06 (s, 3H), 2.01-1.89 (m, 3H), 1.77-1.40 (m, 3H).

Molecular formula: C₂₂H₂₃N₅ Precise molecular weight: 357.20 LC-MS (m/z)=358.12[M+H]⁺.

### Example 48: Synthesis of 2-(4-(((cis)-3-hydroxy-3-methylcyclobutyl) amino)phthalazin-1-yl)-5-(trifluoromethyl)phenol (Compound 129)

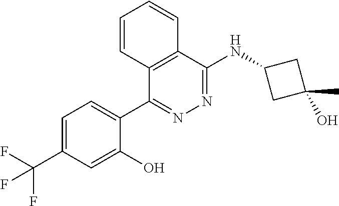 [SMILES: C[C@]1(O)C[C@@H](Nc2nnc(-c3ccc(C(F)(F)F)cc3O)c3ccccc23)C1]

### Step 1: Synthesis of (cis)-3-((4-chlorophthalazin-1-yl)amino)-1-methylcyclobutan-1-ol

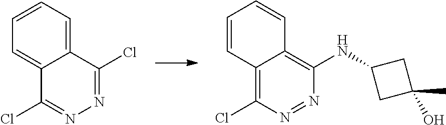 [SMILES: Clc1nnc(Cl)c2ccccc12 ; C[C@]1(O)C[C@@H](Nc2nnc(Cl)c3ccccc23)C1]

1,4-Dichlorophthalazine (1.92 g, 9.68 mmol, 1.0 eq.), (cis)-3-amino-1-methylcyclobutan-1-ol hydrochloride (1.6 g, 11.62 mmol, 1.2 eq.) and N,N-diisopropylethylamine (1.87 g, 14.53 mmol, 1.5 eq.) were added to N,N-dimethylacetamide (20.0 mL), and the mixture was reacted at 120° C. for 12 h. After the reaction was completed as monitored by TLC, water (100.0 mL) was added, followed by extraction with ethyl acetate (100.0 mL). The organic phase was dried over anhydrous sodium sulfate and filtered, and the filtrate was concentrated under reduced pressure. The crude product was purified by silica gel column chromatography (specification of silica gel: 100-200 mesh, dichloromethane:methanol=100:1 to 40:1) to give the product (900.0 mg, yield: 36.0%).

### Step 2: Synthesis of (cis)-3-((4-(2-(ethoxymethoxy)-4-(trifluoromethyl)phenyl)phthalazin-1-yl)amino)-1-methylcyclobutan-1-ol

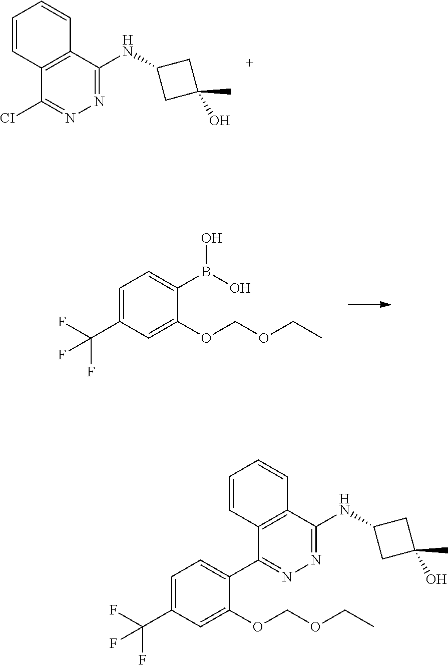

(Cis)-3-((4-chlorophthalazin-1-yl)amino)-1-methylcyclobutan-1-ol (250.0 mg, 0.94 mmol, 1.0 eq.), (2-(ethoxymethoxy)-4-(trifluoromethyl)phenyl)boronic acid (350.3 mg, 1.32 mmol, 1.4 eq.), sodium bicarbonate (159.1 mg, 1.89 mmol, 2.0 eq.) and Pd(dppf)Cl₂ (34.6 mg, 0.04 mmol, 0.05 eq.) were added to a mixed solution of 1,4-dioxane (10.0 mL) and water (5.0 mL), and the mixture was reacted at 110° C. for 2 h in nitrogen atmosphere. After the reaction was completed as monitored by TLC, water (100.0 mL) was added, followed by extraction with ethyl acetate (100.0 mL). The organic phase was dried over anhydrous sodium sulfate and filtered, and the filtrate was concentrated under reduced pressure. The crude product was purified by silica gel column chromatography (dichloromethane:methanol=80:1 to 30:1) to give the product (320.0 mg, yield: 75.0%).

### Step 3: Synthesis of 2-(4-(((cis)-3-hydroxy-3-methylcyclobutyl)amino) phthalazin-1-yl)-5-(trifluoromethyl)phenol

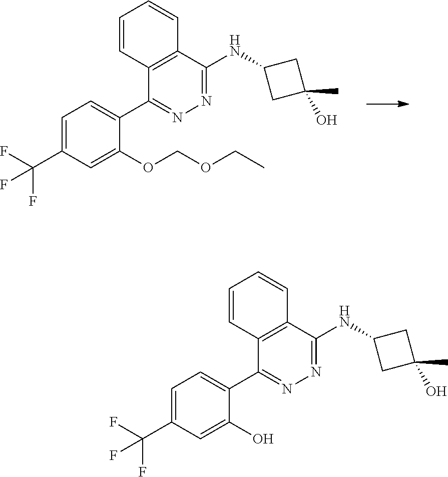 [SMILES: CCOCOc1cc(C(F)(F)F)ccc1-c1nnc(N[C@H]2C[C@@](C)(O)C2)c2ccccc12 ; C[C@]1(O)C[C@@H](Nc2nnc(-c3ccc(C(F)(F)F)cc3O)c3ccccc23)C1]

(Cis)-3-((4-(2-(ethoxymethoxy)-4-(trifluoromethyl)phenyl)phthalazin-1-yl)amino)-1-methylcyclobutan-1-ol (310.0 mg, 0.69 mmol, 1.0 eq.) was added to dichloromethane (2.0 mL), followed by dropwise addition of a hydrogen chloride-1,4-dioxane solution (4.0 mol/L, 4.0 mL). The mixture was reacted at room temperature for 1 h. After the reaction was completed as monitored by TLC, the pH of the system was adjusted to 7-8 with a saturated aqueous sodium bicarbonate solution, followed by extraction with dichloromethane (50.0 mL). The organic phase was dried over anhydrous sodium sulfate and filtered, and the filtrate was concentrated under reduced pressure. The crude product was purified by silica gel column chromatography (specification of silica gel: 100-200 mesh, dichloromethane:methanol=40:1 to 30:1) to give the product (180.0 mg, yield: 66.9%).

¹HNMR (400 MHz, DMSO-d6) δ (ppm): 10.37 (s, 1H), 8.44-8.42 (d, J=8 Hz, 1H), 7.86 (t, J=8 Hz, 1H), 7.77 (t, J=8 Hz, 1H), 7.64-7.62 (d, J=8 Hz, 1H), 7.52-7.50 (d, J=8 Hz, 1H), 7.45-7.43 (d, J=8 Hz, 1H), 7.31-7.28 (m, 2H), 5.02 (s, 1H), 4.27-4.22 (m, 1H), 2.48-2.47 (m, 2H), 2.20-2.15 (m, 2H), 1.34 (s, 3H).

Molecular formula: C₂₀H₁₈F₃N₃O₂ Precise molecular weight: 389.14 LC-MS (m/z)=390.06[M+H]⁺.

### Example 49: Synthesis of (2S,5R)-5-((4-(4-cyano-2-hydroxyphenyl)phthalazin-1-yl)amino)-1-methylpiperidine-2-carboxylic acid (Compound 132)

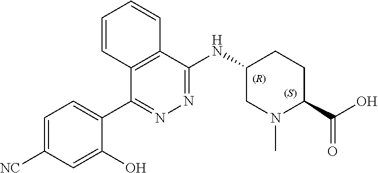

### Step 1: Synthesis of 1-(tert-butyl) 2-ethyl (2S,5R)-5-((4-(4-cyano-2-(ethoxymethoxy)phenyl)phthalazin-1-yl)amino)piperidine-1,2-dicarboxylate

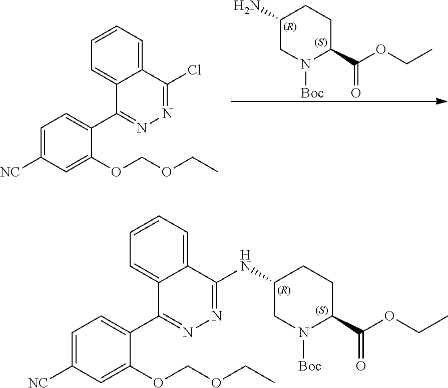

4-(4-Chlorophthalazin-1-yl)-3-(ethoxymethoxy)benzonitrile (310.0 mg, 0.91 mmol, 1.0 eq.), 1-(tert-butyl) 2-ethyl (2S,5R)-5-aminopiperidine-1,2-dicarboxylate (298.1 mg, 1.09 mmol, 1.2 eq.), cesium carbonate (594.2 mg, 1.82 mmol, 2.0 eq.), Pd₂(dba)₃ (83.5 mg, 0.09 mmol, 0.1 eq.) and 1,1′-binaphthyl-2,2′-bisdiphenylphosphine (113.5 mg, 0.18 mmol, 0.2 eq.) were added to 1,4-dioxane (15.0 mL), and the mixture was reacted at 90° C. for 12 h in nitrogen atmosphere. After the reaction was completed as monitored by TLC, water (100.0 mL) was added, followed by extraction with ethyl acetate (100.0 mL). The organic phase was dried over anhydrous sodium sulfate and filtered, and the filtrate was concentrated under reduced pressure. The crude product was purified by silica gel column chromatography (specification of silica gel: 100-200 mesh, dichloromethane:methanol=100:1 to 60:1) to give the product (262.5 mg, yield: 50%).

### Step 2: Synthesis of ethyl (2S,5R)-5-((4-(4-cyano-2-hydroxyphenyl)phthalazin-1-yl)amino)piperidine-2-carboxylate

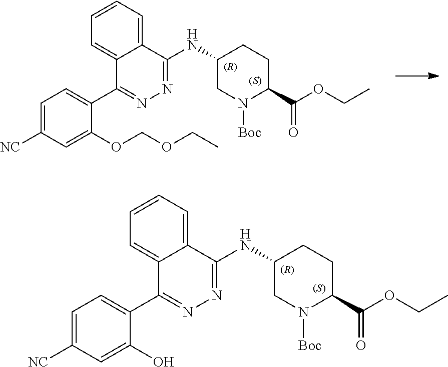

1-(Tert-butyl) 2-ethyl (2S,5R)-5-((4-(4-cyano-2-(ethoxymethoxy)phenyl)phthalazin-1-yl)amino)piperidine-1,2-dicarboxylate (260.0 mg, 0.45 mmol, 1.0 eq.) was added to dichloromethane (2.0 mL), followed by dropwise addition of a hydrogen chloride-1,4-dioxane solution (4.0 mol/L, 4.0 mL). The mixture was reacted at room temperature for 2 h. After the reaction was completed as monitored by TLC, the pH of the system was adjusted to 7-8 with a saturated aqueous sodium bicarbonate solution, followed by extraction with dichloromethane (100.0 mL). The organic phase was dried over anhydrous sodium sulfate and filtered, and the filtrate was concentrated under reduced pressure to give the product (159.4 mg, yield: 90%).

### Step 3: Synthesis of ethyl (2S,5R)-5-((4-(4-cyano-2-hydroxyphenyl)phthalazin-1-yl)amino)-1-methylpiperidine-2-carboxylate

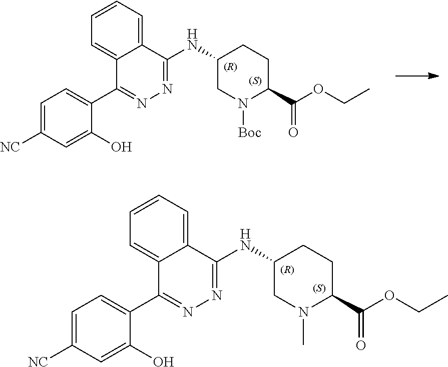

Ethyl (2S,5R)-5-((4-(4-cyano-2-hydroxyphenyl)phthalazin-1-yl)amino)piperidine-2-carboxylate (169.0 mg, 0.40 mmol, 1.0 eq.), an aqueous formaldehyde solution (37%) (46.0 mg, 0.56 mmol, 1.4 eq.) and acetic acid (0.5 mL) were added to methanol (10.0 mL), and the mixture was stirred at room temperature for 0.5 h, followed by addition of sodium cyanoborohydride (38.0 mg, 0.60 mmol, 1.5 eq.). The resulting mixture was reacted at room temperature for 2 h. After the reaction was completed as monitored by TLC, the system was concentrated under reduced pressure, followed by addition of a saturated aqueous sodium bicarbonate solution (100.0 mL). The mixture was stirred for 0.5 h and extracted with dichloromethane (100.0 mL). The organic phase was dried over anhydrous sodium sulfate and filtered, and the filtrate was concentrated under reduced pressure. The crude product was purified by preparative thin-layer chromatography (dichloromethane:methanol=10:1) to give the product (139.4 mg, yield: 80.0%).

### Step 4: Synthesis of (2S,5R)-5-((4-(4-cyano-2-hydroxyphenyl)phthalazin-1-yl)amino)-1-methylpiperidine-2-carboxylic acid

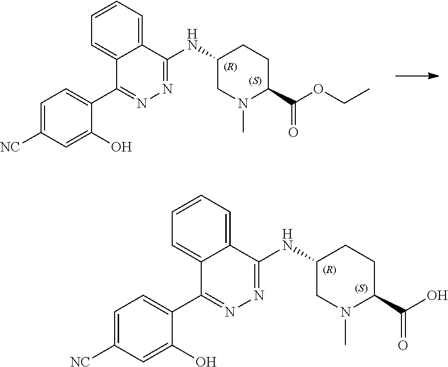

Ethyl (2S,5R)-5-((4-(4-cyano-2-hydroxyphenyl)phthalazin-1-yl)amino)-1-methylpiperidine-2-carboxylate (139.4 mg, 0.32 mmol, 1.0 eq.) and lithium hydroxide (135.5 mg, 3.23 mmol, 10.0 eq.) were added to a mixed solution of methanol (10.0 mL) and water (4.0 mL), and the mixture was stirred at room temperature for 72 h. After the reaction was completed as monitored by TLC, the system was concentrated under reduced pressure and purified by reversed phase column chromatography (acetonitrile:0.1% ammonia solution=4:6) to give the ammonium salt of the product (84.0 mg, yield: 64.4%).

¹HNMR (400 MHz, CD₃OD) δ (ppm): 8.39-8.37 (d, J=8 Hz, 1H), 8.01-7.97 (m, 1H), 7.90 (t, J=8 Hz, 1H), 7.67-7.65 (d, J=8 Hz, 1H), 7.56-7.54 (d, J=8 Hz, 1H), 7.41-7.39 (m, 1H), 7.32 (s, 1H), 4.74-4.68 (m, 1H), 3.99-3.96 (m, 1H), 3.54-3.51 (m, 1H), 3.02-2.93 (m, 4H), 2.52-2.33 (m, 2H), 2.06-1.91 (m, 2H).

Molecular formula: C₂₂H₂₁N₅O₃ Precise molecular weight: 403.16 LC-MS (m/z)=404.13[M+H]⁺.

### Example 50: Synthesis of (R)-3-hydroxy-4-(4-((1-(2-methylpropyl)piperidin-3-yl)amino)phthalazin-1-yl)benzonitrile (Compound 126)

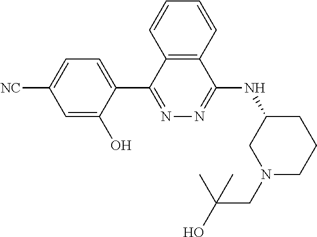 [SMILES: CC(C)(O)CN1CCC[C@@H](Nc2nnc(-c3ccc(C#N)cc3O)c3ccccc23)C1]

### Step 1: Synthesis of (R)-2-(3-((4-chlorophthalazin-1-yl)amino)piperidin-1-yl)-2-methyl-2-propanol

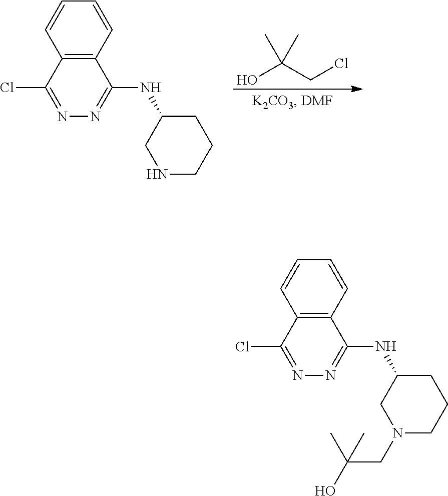

(R)-4-chloro-N-(piperidin-3-yl)phthalazin-1-amine (300 mg, 1.14 mmol, 1.0 eq.), 1-chloro-2-methyl-2-propanol (248 mg, 2.28 mmol, 2.0 eq.) and K₂CO₃ (315 mg, 2.28 mmol, 2.0 eq.) were added to DMF (5 mL), and the mixture was heated to 90° C. and reacted for 24 h. The reaction solution was cooled to room temperature, and water (30 mL) was added to quench the reaction, followed by extraction with EA (30 mL). The organic phase was washed with water (20 mL×3), dried over anhydrous sodium sulfate and filtered, and the filtrate was concentrated under reduced pressure and separated by silica gel column chromatography (DCM:MeOH=100:1 to 20:1) to give the product (240 mg, yield: 62.8%).

### Step 2: Synthesis of (R)-3-(ethoxymethoxy)-4-(4-((1-(2-hydroxy-2-methylpropyl)piperidin-3-yl)amino)phthalazin-1-yl)benzonitrile

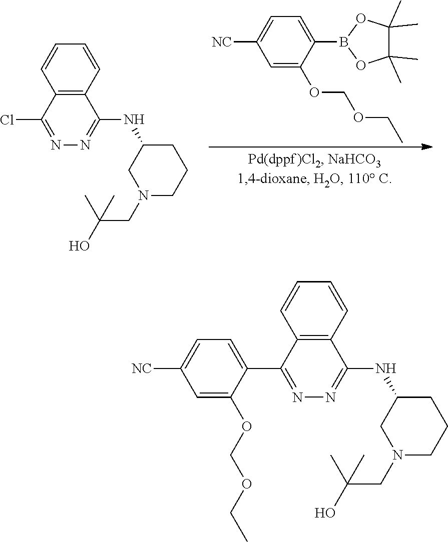 [SMILES: CC(C)(O)CN1CCC[C@@H](Nc2nnc(Cl)c3ccccc23)C1 ; CCOCOc1cc(C#N)ccc1B1OC(C)(C)C(C)(C)O1 ; CCOCOc1cc(C#N)ccc1-c1nnc(N[C@@H]2CCCN(CC(C)(C)O)C2)c2ccccc12]

3-(Ethoxymethoxy)-4-(4,4,5,5-tetramethyl-1,3,2-dioxaborolan-2-yl)benzonitrile (261 mg, 0.860 mmol, 1.2 eq.), (R)-2-(3-((4-chlorophthalazin-1-yl)amino)piperidin-1-yl)-2-methyl-2-propanol (240 mg, 0.717 mmol, 1.0 eq.), Pd(dppf)Cl₂ (52.5 mg, 0.0717 mmol, 0.1 eq.) and NaHCO₃ (120 mg, 1.43 mmol, 2.0 eq.) were added successively to 1,4-dioxane (6 mL), followed by addition of H₂O (3 mL). The mixture was heated to 110° C. and reacted for 2 h in nitrogen atmosphere. The reaction solution was cooled to room temperature, and water (30 mL) was added to quench the reaction, followed by extraction with EA (30 mL×2). The organic phase was dried over anhydrous sodium sulfate and filtered, and the filtrate was concentrated under reduced pressure and separated by silica gel column chromatography (DCM:MeOH=50:1 to 20:1) to give the product (285 mg, yield: 83.6%).

### Step 3: Synthesis of (R)-3-hydroxy-4-(4-((1-(2-methylpropyl)piperidin-3-yl)amino)phthalazin-1-yl)benzonitrile

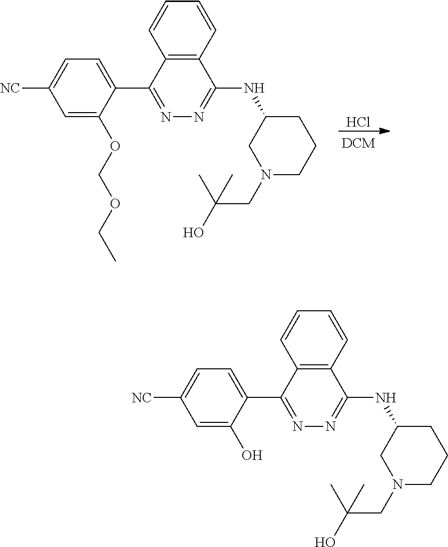 [SMILES: CCOCOc1cc(C#N)ccc1-c1nnc(N[C@@H]2CCCN(CC(C)(C)O)C2)c2ccccc12 ; Cl ; CC(C)(O)CN1CCC[C@@H](Nc2nnc(-c3ccc(C#N)cc3O)c3ccccc23)C1]

(R)-3-(ethoxymethoxy)-4-(4-((1-(2-hydroxy-2-methylpropyl)piperidin-3-yl)amino)phthalazin-1-yl)benzonitrile (285 mg, 0.599 mmol, 1.0 eq.) was dissolved in DCM (3 mL), followed by dropwise addition of a solution of hydrogen chloride in 1,4-dioxane (4 mol/L, 0.75 mL, 3.00 mmol, 5.0 eq.). The mixture was stirred at room temperature for 1 h. The pH was adjusted to 9 with a saturated aqueous Na₂CO₃ solution, followed by extraction with DCM (20 mL×3). The organic phase was dried over anhydrous sodium sulfate and filtered, and the filtrate was concentrated under reduced pressure and separated by silica gel column chromatography (DCM:MeOH=50:1 to 10:1) to give the product (146 mg, yield: 58.4%).

¹HNMR (400 MHz, DMSO-d6) δ (ppm): 10.53 (s, 1H), 8.40 (d, J=8.0 Hz, 1H), 7.89-7.85 (m, 1H), 7.81-7.77 (m, 1H), 7.50 (d, J=8.0 Hz, 1H), 7.43-7.40 (m, 2H), 7.37 (d, J=0.8 Hz, 1H), 7.18 (s, 1H), 4.51 (s, 1H), 4.13 (s, 1H), 3.20 (s, 1H), 2.90 (s, 1H), 2.28 (s, 3H), 1.96 (s, 1H), 1.75-1.51 (m, 3H), 1.14 (s, 6H).

Molecular formula: C₂₄H₂₇N₅O₂ Precise molecular weight: 417.22 LC-MS (Pos, m/z)=418.13 [M+H]⁺.

### Example 51: Synthesis of (R)-3-hydroxy-4-(4-((1-(2-hydroxyethyl)piperidin-3-yl)amino)phthalazin-1-yl)benzonitrile (Compound 127)

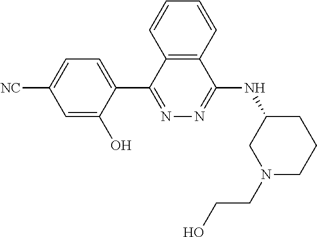 [SMILES: N#Cc1ccc(-c2nnc(N[C@@H]3CCCN(CCO)C3)c3ccccc23)c(O)c1]

### Step 1: Synthesis of (R)-2-(3-((4-chlorophthalazin-1-yl)amino)piperidin-1-yl)-1-ethanol

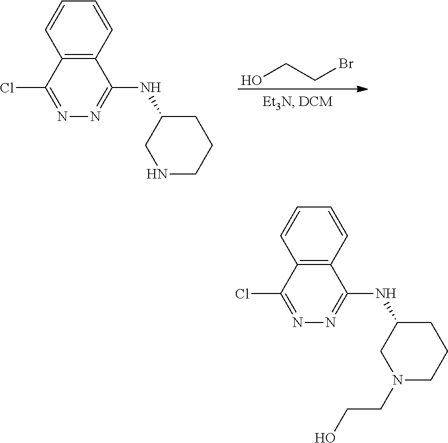

(R)-4-chloro-N-(piperidin-3-yl)phthalazin-1-amine (300 mg, 1.14 mmol, 1.0 eq.), 2-bromoethanol (712 mg, 5.70 mmol, 5.0 eq.) and triethylamine (577 mg, 5.70 mmol, 5.0 eq.) were added to DCM (10 mL), and the mixture was reacted at room temperature for 25 h. Water (20 mL) was added to quench the reaction, followed by extraction with DCM (20 mL). The organic phase was dried over anhydrous sodium sulfate and filtered, and the filtrate was concentrated under reduced pressure and separated by silica gel column chromatography (DCM:MeOH=50:1 to 15:1) to give the product (212 mg, yield: 60.5%).

### Step 2: Synthesis of (R)-3-(ethoxymethoxy)-4-(4-((1-(2-hydroxyethyl)piperidin-3-yl)amino)phthalazin-1-yl)benzonitrile

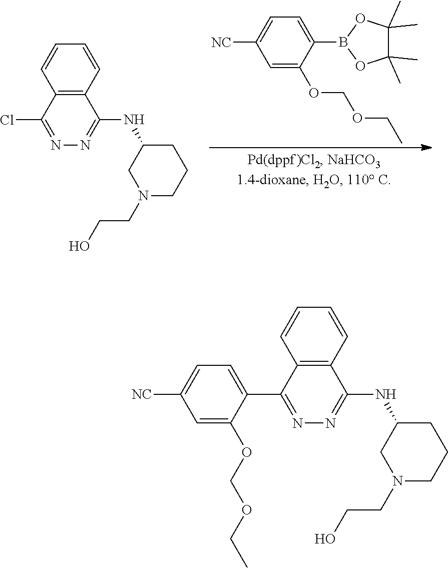 [SMILES: OCCN1CCC[C@@H](Nc2nnc(Cl)c3ccccc23)C1 ; CCOCOc1cc(C#N)ccc1B1OC(C)(C)C(C)(C)O1 ; CCOCOc1cc(C#N)ccc1-c1nnc(N[C@@H]2CCCN(CCO)C2)c2ccccc12]

3-(Ethoxymethoxy)-4-(4,4,5,5-tetramethyl-1,3,2-dioxaborolan-2-yl)benzonitrile (249 mg, 0.822 mmol, 1.2 eq.), (R)-2-(3-((4-chlorophthalazin-1-yl)amino)piperidin-1-yl)-1-ethanol (210 mg, 0.685 mmol, 1.0 eq.), Pd(dppf)Cl₂ (50.1 mg, 0.0685 mmol, 0.1 eq.) and NaHCO₃ (115 mg, 1.37 mmol, 2.0 eq.) were added successively to 1,4-dioxane (6 mL), followed by addition of H₂O (3 mL). The mixture was heated to 110° C. and reacted for 2 h in nitrogen atmosphere. The reaction solution was cooled to room temperature, and water (30 mL) was added to quench the reaction, followed by extraction with EA (30 mL×2). The organic phase was dried over anhydrous sodium sulfate and filtered, and the filtrate was concentrated under reduced pressure and separated by silica gel column chromatography (DCM:MeOH=50:1 to 10:1) to give the product (180 mg, yield: 58.8%).

### Step 3: Synthesis of (R)-3-hydroxy-4-(4-((1-(2-hydroxyethyl)piperidin-3-yl)amino)phthalazin-1-yl)benzonitrile

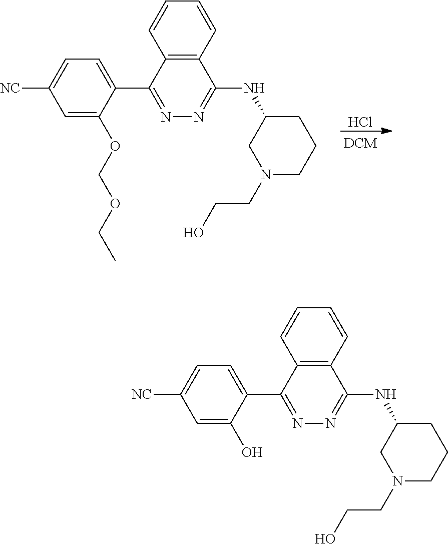 [SMILES: CCOCOc1cc(C#N)ccc1-c1nnc(N[C@@H]2CCCN(CCO)C2)c2ccccc12 ; Cl ; N#Cc1ccc(-c2nnc(N[C@@H]3CCCN(CCO)C3)c3ccccc23)c(O)c1]

(R)-3-(ethoxymethoxy)-4-(4-((1-(2-hydroxyethyl)piperidin-3-yl)amino)phthalazin-1-yl)benzonitrile (180 mg, 0.402 mmol, 1.0 eq.) was dissolved in DCM (3 mL), followed by dropwise addition of a solution of hydrogen chloride in 1,4-dioxane (4 mol/L, 0.50 mL, 2.01 mmol, 5.0 eq.). The mixture was stirred at room temperature for 1 h. The pH was adjusted to 9 with a saturated aqueous Na₂CO₃ solution, followed by extraction with DCM (20 mL×3). The organic phase was dried over anhydrous sodium sulfate and filtered, and the filtrate was concentrated under reduced pressure and separated by reversed phase column chromatography (acetonitrile:0.1% ammonia solution=2:5) to give the product (72.0 mg, yield: 46.0%).

¹HNMR (400 MHz, DMSO-d6) δ (ppm): 10.50 (s, 1H), 8.40 (d, J=8.0 Hz, 1H), 7.88-7.84 (m, 1H), 7.80-7.77 (m, 1H), 7.50 (d, J=8.0 Hz, 1H), 7.43-7.42 (m, 2H), 7.33 (s, 1H), 7.15 (d, J=7.6 Hz, 1H), 4.43 (s, 2H), 3.54-3.51 (m, 2H), 3.17-3.14 (m, 1H), 2.81 (d, J=8.0 Hz, 1H), 2.47-2.44 (m, 2H), 2.11-2.03 (m, 2H), 1.98 (d, J=8.0 Hz, 1H), 1.75 (d, J=12.0 Hz, 1H), 1.61-1.52 (m, 2H).

Molecular formula: C₂₂H₂₃N₅O₂ Precise molecular weight: 389.19 LC-MS (Pos, m/z)=390.12[M+H]⁺.

### Example 52: Synthesis of (S)—N-(4-(4-cyano-2-hydroxyphenyl)phthalazin-1-yl)-1-methylpiperidine-2-carboxamide (Compound 131)

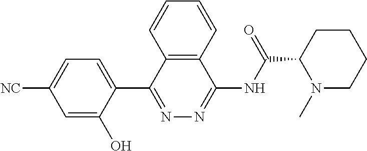 [SMILES: CN1CCCC[C@H]1C(=O)Nc1nnc(-c2ccc(C#N)cc2O)c2ccccc12]

### Step 1: Synthesis of (S)—N-(4-chlorophthalazin-1-yl)-1-methylpiperidine-2-carboxamide

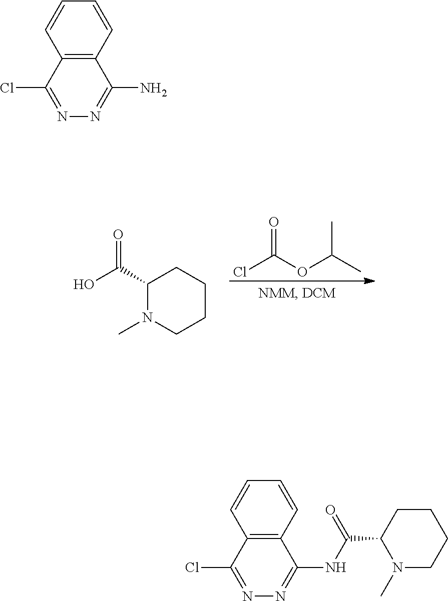

(S)-1-methylpiperidine-2-carboxylic acid (180 mg, 1.26 mmol, 1.0 eq.) was dissolved in DCM (5 mL), followed by addition of N-methylmorpholine (191 mg, 1.89 mmol, 1.5 eq.) and isopropyl chloroformate (232 mg, 1.89 mmol, 1.5 eq.). The mixture was stirred at room temperature for 2 h, followed by addition of 4-chlorophthalazin-1-amine (226 mg, 1.26 mmol, 1.0 eq.). The resulting mixture was stirred at room temperature for 20 h. H₂O (20 mL) was added, followed by extraction with DCM (20 mL). The organic phase was dried over anhydrous sodium sulfate and filtered, and the filtrate was concentrated under reduced pressure and separated by silica gel column chromatography (DCM:MeOH=100:1 to 20:1) to give the product (160 mg, yield: 41.8%).

### Step 2: Synthesis of (S)—N-(4-(4-cyano-2-(ethoxymethoxy)phenyl)phthalazin-1-yl)-1-methylpiperidine-2-carboxamide

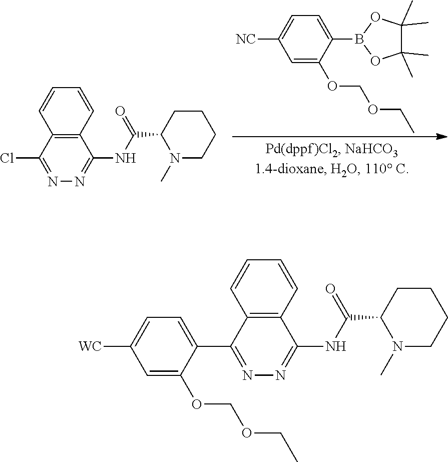

3-(Ethoxymethoxy)-4-(4,4,5,5-tetramethyl-1,3,2-dioxaborolan-2-yl)benzonitrile (191 mg, 0.630 mmol, 1.2 eq.), (S)—N-(4-chlorophthalazin-1-yl)-1-methylpiperidine-2-carboxamide (160 mg, 0.525 mmol, 1.0 eq.), Pd(dppf)Cl₂ (38.4 mg, 0.0525 mmol, 0.1 eq.) and NaHCO₃ (88.2 mg, 1.05 mmol, 2.0 eq.) were added successively to 1,4-dioxane (6 mL), followed by addition of H₂O (3 mL). The mixture was heated to 110° C. and reacted for 2 h in nitrogen atmosphere. The reaction solution was cooled to room temperature, and water (20 mL) was added to quench the reaction, followed by extraction with EA (20 mL×2). The organic phase was dried over anhydrous sodium sulfate and filtered, and the filtrate was concentrated under reduced pressure and separated by silica gel column chromatography (DCM:MeOH=100:1 to 20:1) to give the product (80.0 mg, yield: 34.2%).

### Step 3: Synthesis of (S)—N-(4-(4-cyano-2-hydroxyphenyl)phthalazin-1-yl)-1-methylpiperidine-2-carboxamide

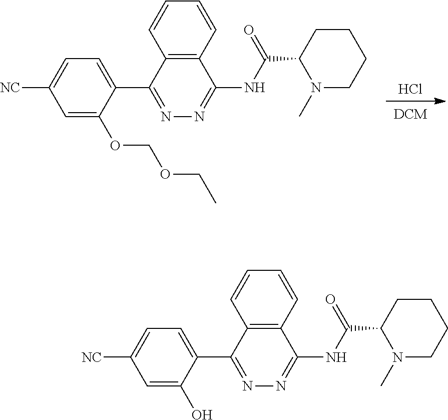

(S)—N-(4-(4-cyano-2-(ethoxymethoxy)phenyl)phthalazin-1-yl)-1-methylpiperidine-2-carboxamide (80.0 mg, 0.182 mmol, 1.0 eq.) was dissolved in DCM (2 mL), followed by dropwise addition of a solution of hydrogen chloride in 1,4-dioxane (4 mol/L, 0.23 mL, 0.90 mmol, 5.0 eq.). The mixture was stirred at room temperature for 1 h. H₂O (5 mL) was added, followed by washing with DCM (5 mL). The aqueous phase was adjusted to pH of 9 with a saturated aqueous Na₂CO₃ solution and extracted with a mixed solvent (DCM:MeOH=10:1) (10 mL×5). The organic phase was dried over anhydrous sodium sulfate and filtered, and the filtrate was concentrated under reduced pressure and separated by preparative thin-layer chromatography (DCM:MeOH=10:1) to give the product (38.2 mg, yield: 54.9%).

¹HNMR (400 MHz, DMSO-d6) δ (ppm): 8.39 (s, 1H), 8.07-7.98 (m, 2H), 7.76 (d, J=8.0 Hz, 1H), 7.58 (d, J=8.0 Hz, 1H), 7.42-7.40 (m, 1H), 7.33 (d, J=1.2 Hz, 1H), 3.25-3.22 (m, 2H), 2.63 (s, 3H), 2.53 (s, 1H), 2.28 (d, J=11.2 Hz, 1H), 1.96-1.89 (m, 2H), 1.87-1.77 (m, 2H), 1.60-1.53 (m, 1H).

Molecular formula: C₂₂H₂₁N₅O₂ Precise molecular weight: 387.17 LC-MS (Pos, m/z)=388.13[M+H]⁺.

### Example 53: Synthesis of (R)-3-hydroxy-4-(7-((1-methylpiperidin-3-yl)amino)thieno[2,3-d]pyridazin-4-yl)benzonitrile (Compound 77) and (R)-3-hydroxy-4-(4-((1-methylpiperidin-3-yl)amino)thieno[2,3-d]pyridazin-7-yl)benzonitrile (Compound 78)

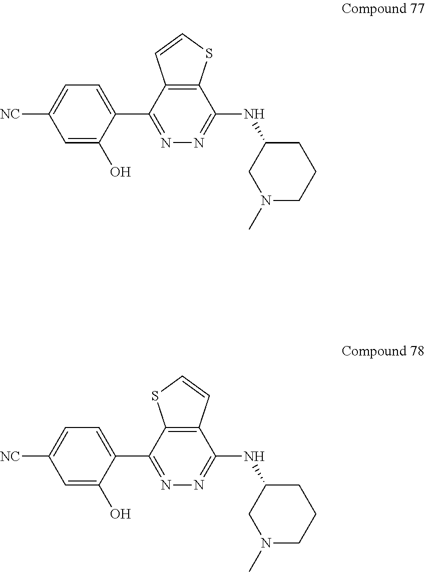 [SMILES: CN1CCC[C@@H](Nc2nnc(-c3ccc(C#N)cc3O)c3ccsc23)C1 ; CN1CCC[C@@H](Nc2nnc(-c3ccc(C#N)cc3O)c3sccc23)C1]

### Step 1: Synthesis of 4-(7-chlorothieno[2,3-d]pyridazin-4-yl)-3-methoxybenzonitrile and 4-(4-chlorothieno[2,3-d]pyridazin-7-yl)-3-methoxybenzonitrile

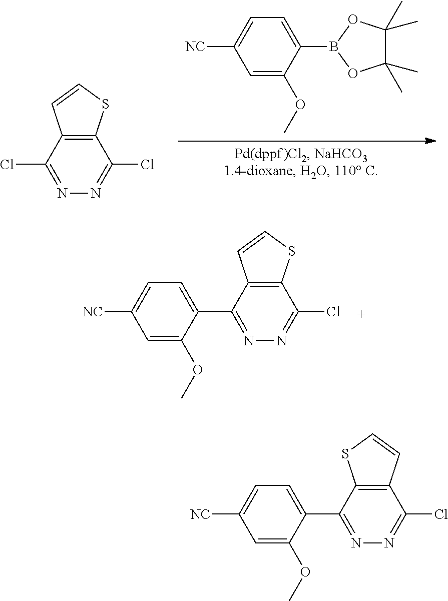

3-Methoxy-4-(4,4,5,5-tetramethyl-1,3,2-dioxaborolan-2-yl)benzonitrile (1.26 g, 4.86 mmol, 1.0 eq.), 4,7-dichlorothieno[2,3-d]pyridazine (997 mg, 4.86 mmol, 1.0 eq.), Pd(dppf)Cl₂ (178 mg, 0.243 mmol, 0.05 eq.) and NaHCO₃ (817 g, 9.72 mmol, 2.0 eq.) were added successively to 1,4-dioxane (16 mL), followed by addition of H₂O (8 mL). The mixture was heated to 110° C. and reacted for 2 h in nitrogen atmosphere. Water (30 mL) was added, followed by extraction with EA (40 mL×2). The organic phase was dried over anhydrous sodium sulfate and filtered, and the filtrate was concentrated under reduced pressure. The crude product was purified and separated by silica gel column chromatography (PE:EA=5:1 to 1:1) to give the product (500 mg, yield: 34.1%).

### Step 2: synthesis of tert-butyl (R)-3-((4-(4-cyano-2-methoxyphenyl)thieno[2,3-d]pyridazin-7-yl)amino)piperidine-1-carboxylate and tert-butyl (R)-3-((7-(4-cyano-2-methoxyphenyl)thieno[2,3-d]pyridazin-4-yl)amino)piperidine-1-carboxylate

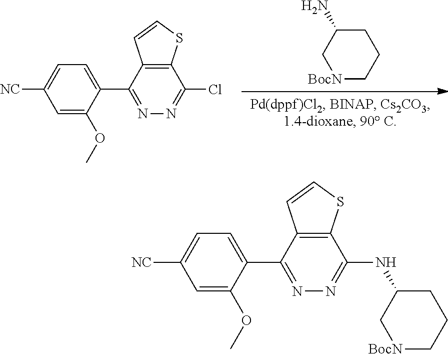

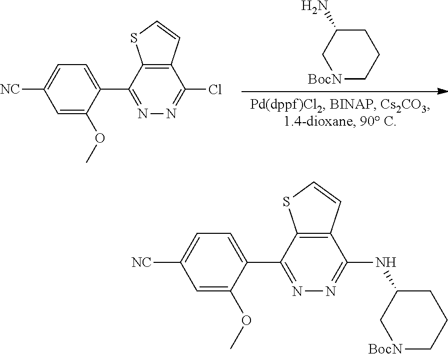

The mixture (500 g, 1.16 mmol, 1.0 eq.) of 4-(7-chlorothieno[2,3-d]pyridazin-4-yl)-3-methoxybenzonitrile and 4-(4-chlorothieno[2,3-d]pyridazin-7-yl)-3-methoxybenzonitrile, tert-butyl (R)-3-aminopiperidine-1-carboxylate (499 mg, 2.49 mmol, 1.5 eq.), Pd₂(dba)₃ (152 mg, 0.166 mmol, 0.1 eq.), BINAP (207 mg, 0.332 mmol, 0.2 eq.) and Cs₂CO₃ (1.05 g, 3.22 mmol, 2.0 eq.) were added successively to 1,4-dioxane (20 mL), and the mixture was heated to 90° C. and reacted for 20 h in nitrogen atmosphere. The reaction solution was cooled to room temperature, and water (40 mL) was added to quench the reaction, followed by extraction with EA (30 mL×2). The organic phase was dried over anhydrous sodium sulfate and filtered, and the filtrate was concentrated under reduced pressure and purified and separated by silica gel column chromatography (DCM:MeOH=100:1 to 20:1) to give the product (610 mg, yield: 79.1%).

### Step 3: Synthesis of (R)-3-hydroxy-4-(7-(piperidin-3-ylamino)thieno[2,3-d]pyridazin-4-yl)benzonitrile and (R)-3-hydroxy-4-(4-(piperidin-3-ylamino)thieno[2,3-d]pyridazin-7-yl)benzonitrile

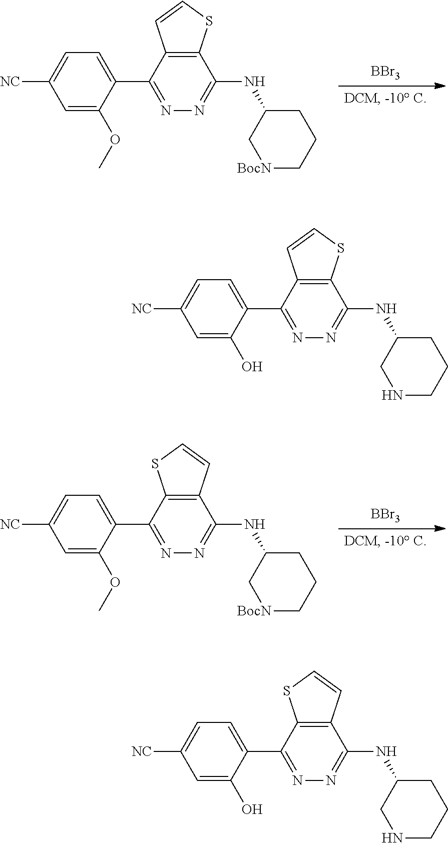

The mixture (600 mg, 1.29 mmol, 1.0 eq.) of tert-butyl (R)-3-((4-(4-cyano-2-methoxyphenyl)thieno[2,3-d]pyridazin-7-yl)amino)piperidine-1-carboxylate and tert-butyl (R)-3-((7-(4-cyano-2-methoxyphenyl)thieno[2,3-d]pyridazin-4-yl)amino)piperidine-1-carboxylate was dissolved in DCM (15 mL), and the mixture was cooled to −10° C., followed by dropwise addition of BBr₃ (1.29 g, 5.16 mmol, 4.0 eq.). The resulting mixture was reacted for 5 h. MeOH (5 mL) was added to quench the reaction, and the pH was adjusted to 8 with a saturated aqueous NaHCO₃ solution, followed by extraction with DCM (30 mL×2). The organic phase was dried over anhydrous sodium sulfate and filtered, and the filtrate was concentrated under reduced pressure to give the product (280 mg, yield: 61.8%).

### Step 4: Synthesis of (R)-3-hydroxy-4-(7-((1-methylpiperidin-3-yl)amino)thieno[2,3-d]pyridazin-4-yl)benzonitrile and (R)-3-hydroxy-4-(4-((1-methylpiperidin-3-yl)amino)thieno[2,3-d]pyridazin-7-yl)benzonitrile

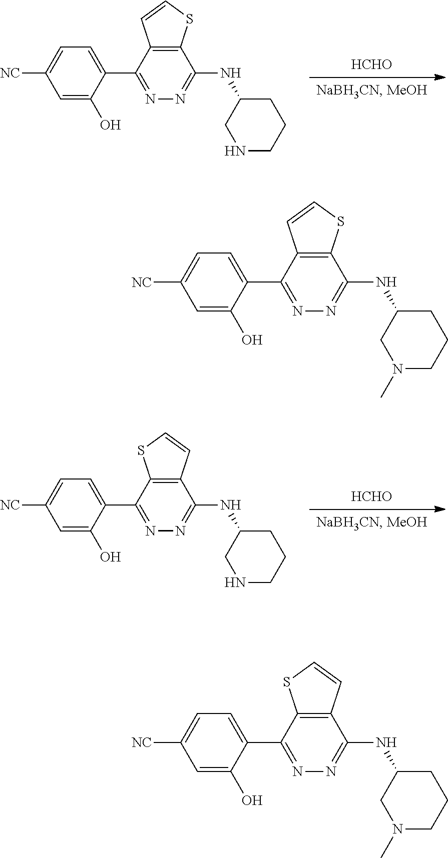

The mixture (200 mg, 0.596 mmol, 1.0 eq.) of (R)-3-hydroxy-4-(7-(piperidin-3-ylamino)thieno[2,3-d]pyridazin-4-yl)benzonitrile and (R)-3-hydroxy-4-(4-(piperidin-3-ylamino)thieno[2,3-d]pyridazin-7-yl)benzonitrile was dissolved in MeOH (4 mL), followed by addition of an aqueous formaldehyde solution (37%) (139 mg, 1.71 mmol, 3.0 eq.). The mixture was stirred at room temperature for 30 min. NaBH₃CN (107 mg, 1.71 mmol, 3.0 eq.) was then added and the mixture was reacted for 1 h. A saturated aqueous NaHCO₃ solution was added to quench the reaction, followed by extraction with DCM (20 mL×3). The organic phase was dried over anhydrous sodium sulfate and filtered, and the filtrate was concentrated under reduced pressure. The crude product was separated by preparative thin-layer chromatography (DCM:MeOH=10:1) to give (R)-3-hydroxy-4-(7-((1-methylpiperidin-3-yl)amino)thieno[2,3-d]pyridazin-4-yl)benzonitrile (22.0 mg, yield: 10.6%) and (R)-3-hydroxy-4-(4-((1-methylpiperidin-3-yl)amino)thieno[2,3-d]pyridazin-7-yl)benzonitrile (23.0 mg, yield: 11.1%).

### (R)-3-hydroxy-4-(7-((1-methylpiperidin-3-yl)amino)thieno[2,3-d]pyridazin-4-yl)benzonitrile

¹HNMR (400 MHz, DMSO-d6) δ (ppm): 8.11 (d, J=5.2 Hz, 1H), 7.71 (d, J=8.4 Hz, 1H), 7.41-7.38 (m, 3H), 7.19 (d, J=7.6 Hz, 1H), 4.41-4.39 (m, 1H), 3.05 (d, J=7.6 Hz, 1H), 2.70 (d, J=10.8 Hz, 1H), 2.21 (s, 3H), 1.97-1.92 (m, 3H), 1.75-1.71 (m, 1H), 1.64-1.55 (m, 1H), 1.49-1.35 (m, 1H).

Molecular formula: C₁₉H₁₉N₅OS Precise molecular weight: 365.13 LC-MS (m/z): 366.08[M+H]⁺

### (R)-3-hydroxy-4-(4-((1-methylpiperidin-3-yl)amino)thieno[2,3-d]pyridazin-7-yl)benzonitrile

¹HNMR (400 MHz, DMSO-d6) δ (ppm): 8.25 (d, J=5.2 Hz, 1H), 8.12 (d, J=4.8 Hz, 1H), 7.92 (d, J=8.4 Hz, 1H), 7.60 (s, 1H), 7.45 (d, J=6.8 Hz, 2H), 4.48 (s, 1H), 3.20 (d, J=8.8 Hz, 1H), 2.86 (d, J=10.4 Hz, 1H), 2.37 (s, 3H), 2.23 (s, 2H), 2.01-1.98 (m, 3H), 1.82 (s, 1H), 1.72-1.63 (m, 1H), 1.54 (s, 1H).

Molecular formula: C₁₉H₁₉N₅OS Precise molecular weight: 365.13 LC-MS (m/z): 366.14[M+H]⁺

### Example 54: Synthesis of Compound (R)-5-methyl-2-(1-((1-methylpiperidin-3-yl)amino)pyrido[3,4-d]pyridazin-4-yl)phenol (Compound 49)

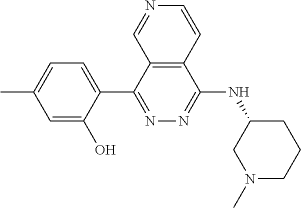 [SMILES: Cc1ccc(-c2nnc(N[C@@H]3CCCN(C)C3)c3ccncc23)c(O)c1]

### Step 1: Synthesis of Intermediate 4-(2-methoxy-4-methylbenzoyl)nicotinic acid

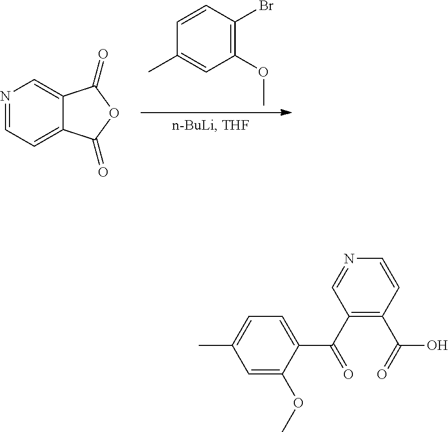

1-Bromo-2-methoxy-4-methylbenzene (20.96 g, 104.22 mmol, 1.05 eq.) was dissolved in anhydrous tetrahydrofuran (150 mL), followed by dropwise addition of n-butyllithium (48 mL, 119.11 mmol, 1.2 eq.) at −78° C. After the completion of dropwise addition, the reaction solution was added dropwise to a solution of 3,4-pyridine dicarboxylic anhydride (14.8 g, 99.26 mmol, 1.0 eq.) in tetrahydrofuran (150 mL) at −78° C. After the completion of dropwise addition, the mixture was reacted at room temperature for 1 h. After the reaction was completed as detected by TLC, the reaction solution was poured into a mixed solution of ice water (200 mL) and 2 mol/L hydrochloric acid (200 mL). The mixture was concentrated under reduced pressure and extracted with dichloromethane (200 mL×3). The organic phases were combined, dried and concentrated to give the crude product (13.7 g), which was directly used in the next step.

### Step 2: Synthesis of Intermediate 4-(2-methoxy-4-methylphenyl)pyrido[3,4-d]pyridazin-1-ol

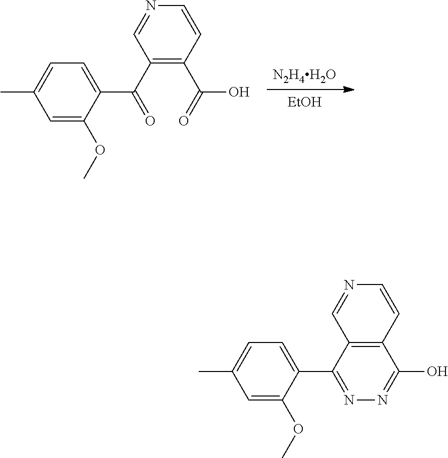 [SMILES: "EtOH" ; COc1cc(C)ccc1C(=O)c1cnccc1C(=O)O ; "N2H4·H2O" ; COc1cc(C)ccc1-c1nnc(O)c2ccncc12]

The mixture obtained in the previous step was dissolved in ethanol (120 mL), followed by addition of 85% hydrazine hydrate (3.97 g, 67.4 mmol, 1.5 eq.). The mixture was heated to 80° C. and reacted for 2 h. After the reaction was completed as detected by LC-MS, the reaction solution was cooled to room temperature, and solids precipitated. The mixture was filtered, and the filtrate was concentrated and purified by silica gel column chromatography (dichloromethane:methanol=1200:1 to 200:1) to give the product (2.35 g, two-step yield: 8.8%).

### Step 3: Synthesis of Intermediate 1-chloro-4-(2-methoxy-4-methylphenyl)pyrido[3,4-d]pyridazine

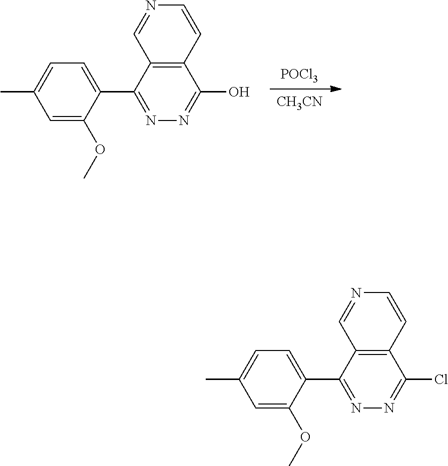

Acetonitrile (20 mL) and phosphorus oxychloride (2.29 g, 14.96 mmol, 2.0 eq.) were added to 4-(2-methoxy-4-methylphenyl)pyrido[3,4-d]pyridazin-1-ol (2 g, 7.48 mmol, 1.0 eq.), and the mixture was reacted at 100° C. for 16 h. After there remained some of the materials as detected by TLC, the reaction solution was supplemented with phosphorus oxychloride (2.29 g, 14.96 mmol, 2.0 eq.) and reacted at 100° C. for 1 h. After the reaction was almost completed as detected by TLC, the reaction solution was concentrated, and the concentrated solution was poured into ice water (10 mL), followed by addition of dichloromethane (20 mL). The pH was adjusted to low alkalinity with sodium bicarbonate, followed by extraction with dichloromethane (20 mL×3). The organic phases were combined, dried, concentrated and purified by silica gel column chromatography (petroleum ether:ethyl acetate=6:1) to give the product (955 mg, yield: 44.6%).

### Step 4: Synthesis of Intermediate tert-butyl (R)-3-((4-(2-methoxy-4-methylphenyl)pyrido[3,4-d]pyridazin-1-yl)amino)piperidine-1-carboxylate

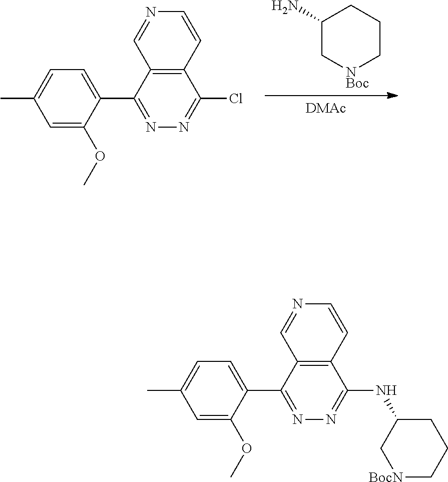

1-Chloro-4-(2-methoxy-4-methylphenyl)pyrido[3,4-d]pyridazine (955 mg, 3.34 mmol, 1.0 eq.) was dissolved in DMAc (10 mL), followed by addition of (R)-1-tert-butoxycarbonyl-3-aminopiperidine (1.34 g, 6.68 mmol, 2.0 eq.). The mixture was reacted at 120° C. for 20 h. After the reaction was completed as detected by TLC, the reaction solution was poured into water (20 mL), followed by extraction with ethyl acetate (20 mL×2). The organic phases were combined, dried and concentrated, and the crude product was purified by silica gel column chromatography (petroleum ether:ethyl acetate=5:1 to 1:1) to give the product (980 mg, yield: 65.3%).

### Step 5: Synthesis of Intermediate (R)-5-methyl-2-(1-((piperidin-3-yl)amino)pyrido[3,4-d]pyridazin-4-yl)phenol

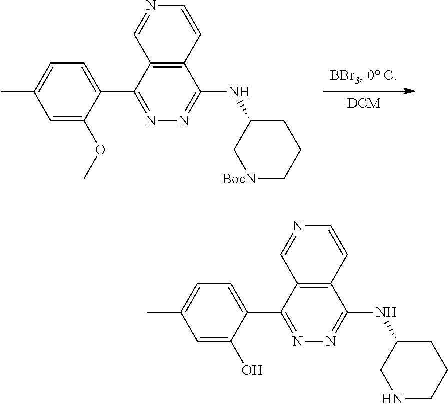

Tert-butyl (R)-3-((4-(2-methoxy-4-methylphenyl)pyrido[3,4-d]pyridazin-1-yl)amino)piperidine-1-carboxylate (980 mg, 2.18 mmol, 1.0 eq.) was dissolved in dichloromethane (10 mL), followed by dropwise addition of boron tribromide (1.64 g, 6.54 mmol, 3.0 eq.) at 0° C. The mixture was reacted at room temperature for 2 h. After there remained some of the materials as detected by TLC, the reaction solution was supplemented with boron tribromide (1.64 g, 6.54 mmol, 3.0 eq.). The resulting mixture was reacted at room temperature for 2 h. After the reaction was almost completed as detected by TLC, methanol (5 mL) and water (10 mL) were added dropwise, slowly and successively to the reaction solution under an ice bath, followed by back-extraction with dichloromethane (20 mL). The pH of the aqueous phase was adjusted to alkalinity with a saturated aqueous sodium bicarbonate solution, followed by extraction with dichloromethane (10 mL). The aqueous phase was concentrated, dissolved in a mixed solution of dichloromethane/methanol, dried over anhydrous magnesium sulfate and concentrated, and the crude product was directly used in the next step.

### Step 6: Synthesis of compound (R)-5-methyl-2-(1-((1-methylpiperidin-3-yl)amino)pyrido[3,4-d]pyridazin-4-yl)phenol

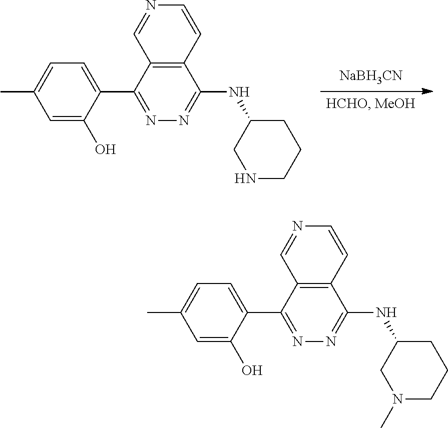

(R)-5-methyl-2-(1-((piperidin-3-yl)amino)pyrido[3,4-d]pyridazin-4-yl)phenol (474 mg, 1.41 mmol, 1.0 eq.) was dissolved in methanol (5 mL), followed by addition of an aqueous formaldehyde solution (37%) (112 mg, 1.41 mmol, 1.0 eq.) and sodium cyanoborohydride (97 mg, 1.55 mmol, 1.1 eq.). The mixture was reacted at room temperature for 5 min. After the reaction was completed as detected by TLC, the reaction solution was concentrated, and the crude product was purified by preparative thin-layer chromatography (dichloromethane:methanol=7:1) to give the product (110 mg, two-step yield: 22.3%).

¹H-NMR (300 MHz, DMSO-d6) δ (ppm): 9.71 (brs, 1H), 8.94-8.92 (d, 1H), 8.87 (s, 1H), 8.30-8.27 (d, 1H), 7.42-7.40 (d, 1H), 7.27-7.25 (d, 1H), 6.83-6.80 (d, 2H), 4.47 (s, 1H), 3.27-3.18 (m, 2H), 2.90-2.88 (m, 1H), 2.39-2.20 (m, 7H), 2.04-1.83 (m, 2H), 1.73-1.52 (m, 2H).

Molecular formula: C₂₀H₂₃N₅O Precise molecular weight: 349.19 LC-MS (m/z): 350.10 [M+H]⁺

### Example 55: Synthesis of Compound (R)-2-(6-chloro-4-((1-methylpiperidin-3-yl)amino)phthalazin-1-yl)-5-methylphenol (Compound 12)

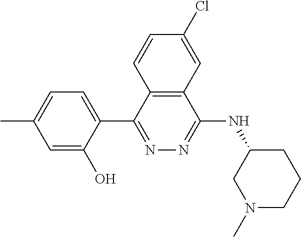 [SMILES: Cc1ccc(-c2nnc(N[C@@H]3CCCN(C)C3)c3cc(Cl)ccc23)c(O)c1]

### Step 1: Synthesis of Intermediate 4-(2-methoxy-4-methylbenzoyl)nicotinic acid

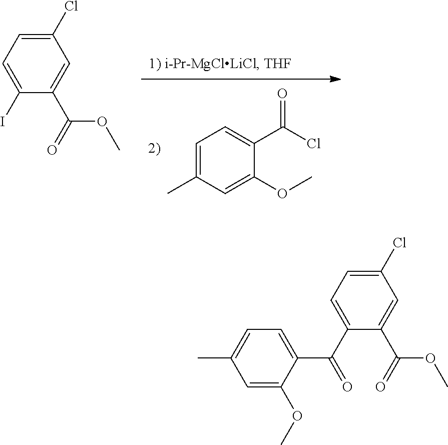

Methyl 5-chloro-2-iodobenzoate (10.3 g, 34.67 mmol, 1.0 eq.) was dissolved in anhydrous tetrahydrofuran (100 mL), followed by dropwise addition of a 1.3 mol/L solution of an isopropylmagnesium chloride lithium chloride complex in tetrahydrofuran (32 mL, 41.6 mmol, 1.2 eq.) at −28° C. After the completion of dropwise addition, the mixture was reacted at this temperature for 30 min. At −78° C., the reaction solution was added dropwise to a solution of 2-methoxy-4-methylbenzoyl chloride (9.6 g, 52 mmol, 1.5 eq.) in tetrahydrofuran (100 mL). After the completion of dropwise addition, the mixture was reacted at room temperature for 1 h. After the reaction was completed as detected by TLC, the reaction solution was concentrated and poured into ice water (100 mL), and potassium carbonate was added, followed by extraction with dichloromethane (200 mL×3). The organic phases were combined, dried and concentrated to give the product (10.3 g, yield: 93.2%).

### Step 2: Synthesis of Intermediate 7-chloro-4-(2-methoxy-4-methylphenyl)phthalazin-1(2H)-one

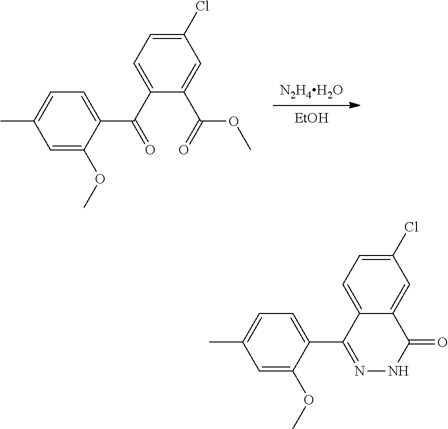

4-(2-Methoxy-4-methylbenzoyl)nicotinic acid (10.3 g, 32.3 mmol, 1.0 eq.) was dissolved in ethanol (100 mL), followed by addition of 85% hydrazine hydrate (2.86 g, 48.45 mmol, 1.5 eq.). The mixture was heated to 90° C. and reacted for 3 h. After the reaction was completed as detected by TLC, the reaction solution was cooled to room temperature, and solids precipitated. The mixture was filtered, and the filter cake was the product (1.5 g, yield: 15.4%).

### Step 3: Synthesis of Intermediate 4,6-dichloro-1-(2-methoxy-4-methylphenyl)phthalazine

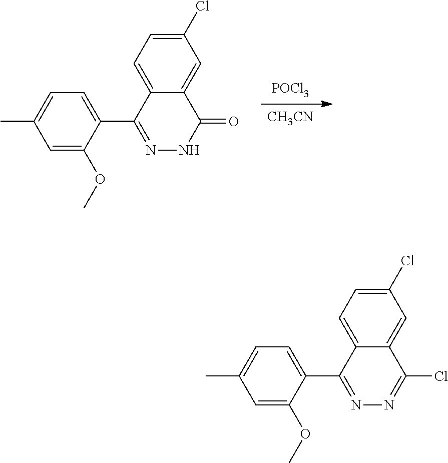

Acetonitrile (10 mL) and phosphorus oxychloride (1.53 g, 9.98 mmol, 2.0 eq.) were added to 7-chloro-4-(2-methoxy-4-methylphenyl)phthalazin-1(2H)-one (1.5 g, 4.99 mmol, 1.0 eq.), and the mixture was reacted at 90° C. for 3 h. After the reaction was completed as detected by TLC, the reaction solution was concentrated, the concentrated solution was poured into ice water (10 mL), and sodium bicarbonate was added to adjust the pH to low alkalinity, followed by extraction with dichloromethane (10 mL×3). The organic phases were combined, dried and concentrated to give the product (1.31 g, yield: 82.3%).

### Step 4: Synthesis of Intermediate tert-butyl (R)-3-((7-(2-methoxy-4-methylphenyl)phthalazin-1-yl)amino)piperidine-1-carboxylate

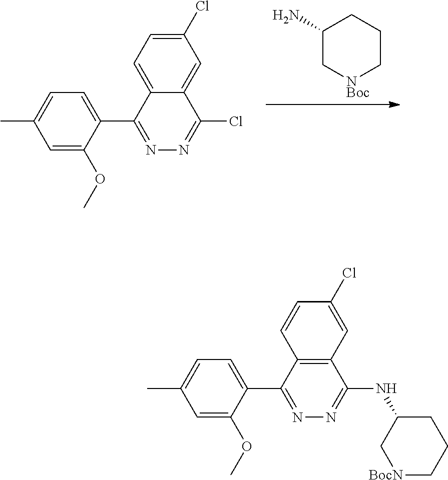 [SMILES: COc1cc(C)ccc1-c1nnc(Cl)c2cc(Cl)ccc12 ; CC(C)(C)OC(=O)N1CCC[C@@H](N)C1 ; COc1cc(C)ccc1-c1nnc(N[C@@H]2CCCN(C(=O)OC(C)(C)C)C2)c2cc(Cl)ccc12]

4,6-Dichloro-1-(2-methoxy-4-methylphenyl)phthalazine (1.31 g, 4.1 mmol, 1.0 eq.) was dissolved in DMAc (10 mL), (R)-1-tert-butyloxycarbonyl-3-aminopiperidine (1.64 g, 8.2 mmol, 2.0 eq.) was added, and the reaction solution was reacted at 120° C. for 24 h. After the reaction was completed as detected by TLC, the reaction solution was poured into water (20 mL), followed by extraction with ethyl acetate (20 mL×2). The organic phases were combined, dried, and concentrated, and the crude product was purified by silica gel column chromatography (petroleum ether:ethyl acetate=10:1 to 1:1) to give the product (870 mg, yield: 40.9%).

### Step 5: Synthesis of Intermediate (R)-2-(6-chloro-4-((piperidin-3-yl)amino)phthalazin-1-yl)-5-methylphenol

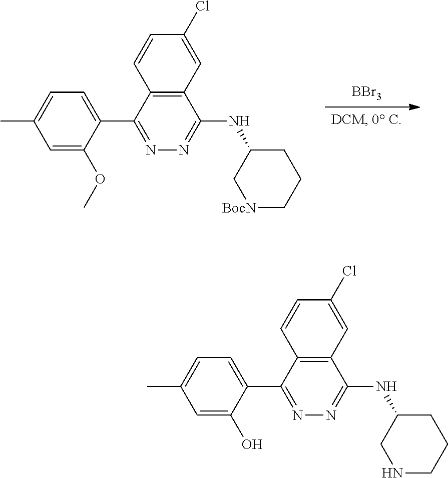 [SMILES: COc1cc(C)ccc1-c1nnc(N[C@@H]2CCCN(C(=O)OC(C)(C)C)C2)c2cc(Cl)ccc12 ; "BBr3" ; "DCM, 0° C." ; Cc1ccc(-c2nnc(N[C@@H]3CCCNC3)c3cc(Cl)ccc23)c(O)c1]

Tert-butyl (R)-3-((7-(2-methoxy-4-methylphenyl)phthalazin-1-yl)amino)piperidine-1-carboxylate (300 mg, 0.621 mmol, 1.0 eq.) was dissolved in dichloromethane (5 mL), boron tribromide (467 mg, 1.863 mmol, 3.0 eq.) was added dropwise at 0° C., and the mixture was reacted at room temperature for 30 min. After the reaction was completed as detected by TLC, methanol (5 mL) and water (5 mL) were added dropwise and slowly to the reaction solution under an ice bath, followed by back-extraction with dichloromethane (20 mL×1, 10 mL×3). Sodium bicarbonate was added to the aqueous phase to adjust the pH to alkalinity, followed by extraction with dichloromethane:methanol=10:1 (10 mL×3), and the organic phases were combined, dried over anhydrous magnesium sulfate, and concentrated to give the product (78 mg, yield: 34%).

### Step 6: Synthesis of compound (R)-2-(6-chloro-4-((1-methylpiperidin-3-yl)amino)phthalazin-1-yl)-5-methylphenol

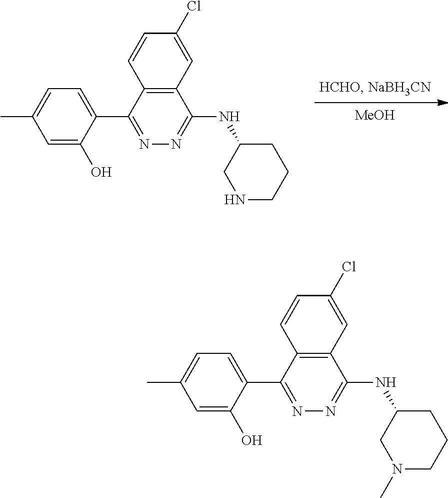

(R)-2-(6-chloro-4-((piperidin-3-yl)amino)phthalazin-1-yl)-5-methylphenol (78 mg, 0.211 mmol, 1.0 eq.) was dissolved in methanol (1 mL), followed by addition of an aqueous formaldehyde solution (37%) (17 mg, 0.211 mmol, 1.0 eq.) and sodium cyanoborohydride (15 mg, 0.232 mmol, 1.1 eq.), and the resulting mixture was reacted at room temperature for 5 min. After the reaction was completed as detected by TLC, the reaction solution was concentrated, and the crude product was purified by preparative thin-layer chromatography (dichloromethane:methanol=7:1) to give the product (27 mg, yield: 33.3%).

¹H-NMR (300 MHz, DMSO-d6) δ (ppm): 9.61 (brs, 1H), 8.62 (s, 1H), 7.85-7.82 (dd, 1H), 7.53-7.50 (d, 2H), 7.14-7.17 (d, 1H), 6.82-6.76 (m, 2H), 4.65 (s, 1H), 3.60-3.56 (m, 3H), 2.73 (s, 4H), 2.32 (s, 3H), 2.03-2.01 (m, 2H), 1.83-1.80 (m, 2H).

Molecular formula: C₂₁H₂₃C₁N₄O Precise molecular weight: 382.16 LC-MS (m/z): 383.09/385.03 [M+H]⁺.

### Example 56: Synthesis of (R)-5-methyl-2-(4-((1-methylpiperidin-3-yl)amino)-5,6,7,8-tetrahydrophthalazin-1-yl)phenol (Compound 1a)

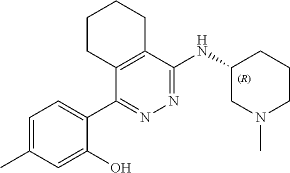

### Step 1: Synthesis of 2-(2-methoxy-4-methylbenzoyl)cyclohex-1-ene-1-carboxylic acid

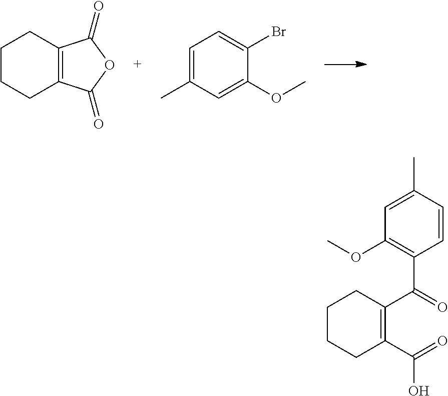

1-Bromo-2-methoxy-4-methylbenzene (10.0 g, 49.73 mmol, 1.0 eq.) was added to tetrahydrofuran (30.0 mL), and the mixture was cooled to −65° C. in nitrogen atmosphere, followed by dropwise and slow addition of a 2.5 mol/L solution of n-butyllithium in n-hexane (19.9 mL, 49.73 mmol, 1.0 eq.). The reaction solution was reacted for 1 h, and added slowly and dropwise to a solution of 4,5,6,7-tetrahydroisobenzofuran-1,3-dione (7.56 g, 49.73 mmol, 1.0 eq.) in tetrahydrofuran (30.0 mL) at −65° C., and the solution was warmed to room temperature and reacted for 12 h. After the reaction was completed as monitored by TLC, a 2 mol/L aqueous HCl solution was added to the system with stirring for 0.5 h, followed by extraction with ethyl acetate (200.0 mL). The organic phase was dried over anhydrous sodium sulfate and filtered, the filtrate was concentrated under reduced pressure, and the crude product was slurried with petroleum ether (100.0 mL) for 2 h, and filtered under vacuum. The filter cake was dried to give the product (5.88 g, yield: 43.1%).

### Step 2: Synthesis of methyl 2-(2-methoxy-4-methylbenzoyl)cyclohex-1-ene-1-carboxylate

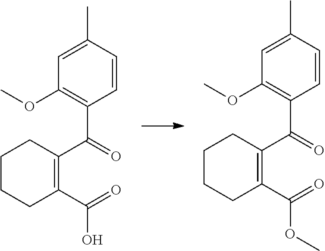

2-(2-Methoxy-4-methylbenzoyl)cyclohex-1-ene-1-carboxylic acid (5.88 g, 21.43 mmol, 1.0 eq.) was added to N,N-dimethylformamide (20.0 mL), followed by addition of potassium carbonate (3.85 g, 27.85 mmol, 1.3 eq.) and iodomethane (3.65 g, 25.71 mmol, 1.2 eq.), and the resulting mixture was reacted at room temperature for 2 h. After the reaction was completed as monitored by TLC, ethyl acetate (200.0 mL) was added, followed by washing with water (100.0 mL×4). The organic phase was dried over anhydrous sodium sulfate and filtered, and the filtrate was concentrated under reduced pressure to give the product (5.24 g, yield: 84.7%).

### Step 3: Synthesis of 4-(2-methoxy-4-methylphenyl)-5,6,7,8-tetrahydrophthalazin-1-ol

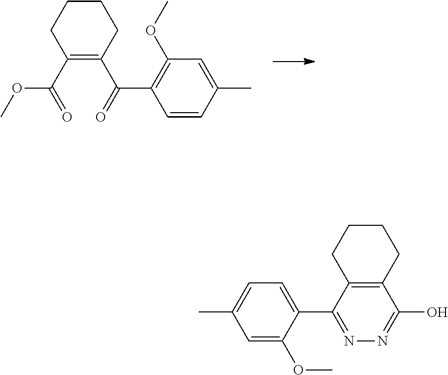

Methyl 2-(2-methoxy-4-methylbenzoyl)cyclohex-1-ene-1-carboxylate (5.24 g, 18.17 mmol, 1.0 eq.) was added to ethanol (80.0 mL), followed by addition of hydrazine hydrate (9.0 g, 181.7 mmol, 10.0 eq.), and the mixture was reacted at 80° C. for 12 h. After the reaction was completed as monitored by TLC, the system was concentrated under reduced pressure, and the crude product was separated by silica gel column chromatography (specification of silica gel: 100-200 mesh, petroleum ether:ethyl acetate=10:1 to 2:1) to give the product (820.0 mg, yield: 16.7%).

### Step 4: Synthesis of 1-chloro-4-(2-methoxy-4-methylphenyl)-5,6,7,8-tetrahydrophthalazine

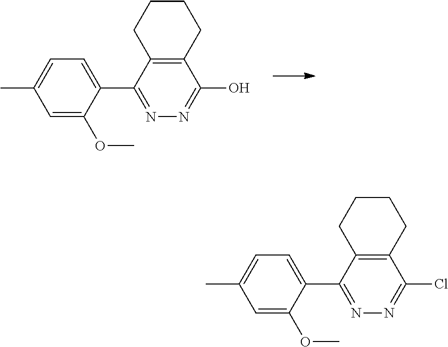 [SMILES: COc1cc(C)ccc1-c1nnc(O)c2c1CCCC2 ; COc1cc(C)ccc1-c1nnc(Cl)c2c1CCCC2]

4-(2-Methoxy-4-methylphenyl)-5,6,7,8-tetrahydrophthalazin-1-ol (820.0 mg, 3.03 mmol, 1.0 eq.) was added to acetonitrile (10.0 mL), followed by addition of phosphorus oxychloride (2.78 g, 18.19 mmol, 6.0 eq.), and the mixture was stirred at 88° C. for 6 h. After the reaction was completed as monitored by TLC, the reaction solution was cooled to room temperature, and a saturated aqueous sodium bicarbonate solution was added to quench the reaction, followed by addition of ethyl acetate (100.0 mL) for extraction. The organic phase was dried over anhydrous sodium sulfate and filtered, the filtrate was concentrated under reduced pressure, and the crude product was purified by silica gel column chromatography (specification of silica gel: 100-200 mesh, petroleum ether:ethyl acetate=2:1) to give the product (600.0 mg, yield: 68.5%).

### Step 5: Synthesis of 2-(4-chloro-5,6,7,8-tetrahydrophthalazin-1-yl)-5-methylphenol

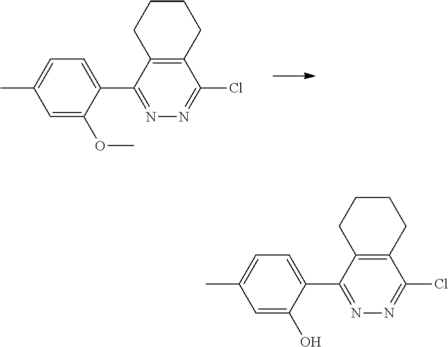 [SMILES: COc1cc(C)ccc1-c1nnc(Cl)c2c1CCCC2 ; Cc1ccc(-c2nnc(Cl)c3c2CCCC3)c(O)c1]

1-Chloro-4-(2-methoxy-4-methylphenyl)-5,6,7,8-tetrahydrophthalazine (600.0 mg, 4.13 mmol, 1.0 eq.) was added to dichloromethane (10.0 mL), the mixture was cooled to −65° C., followed by dropwise and slow addition of boron tribromide (1.56 g, 6.23 mmol, 3.0 eq.), and the reaction solution was warmed to room temperature and stirred for 3 h. After the reaction was completed as monitored by TLC, methanol was added to the system to quench the reaction, followed by concentration under reduced pressure, and a saturated aqueous sodium bicarbonate solution was added to adjust the pH to 7-8, followed by extraction with dichloromethane (100.0 mL). The organic phase was dried over anhydrous sodium sulfate and filtered, and the filtrate was concentrated under reduced pressure to give the product (400.0 mg, yield: 70.4%).

### Step 6: Synthesis of tert-butyl (R)-3-((4-(2-hydroxy-4-methylphenyl)-5,6,7,8-tetrahydrophthalazin-1-yl)amino)piperidine-1-carboxylate

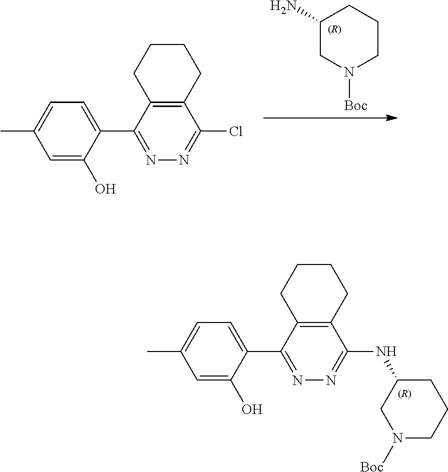

2-(4-Chloro-5,6,7,8-tetrahydrophthalazin-1-yl)-5-methylphenol (370.0 mg, 1.34 mmol, 1.0 eq.), tert-butyl (R)-3-aminopiperidine-1-carboxylate (539.4 mg, 2.68 mmol, 2.0 eq.), sodium tert-butoxide (386.3 mg, 4.02 mmol, 3.0 eq.), Pd₂(dba)₃ (368.1 mg, 0.40 mmol, 0.3 eq.) and 1,1′-binaphthyl-2,2′-bisdiphenylphosphine (375.1 mg, 0.60 mmol, 0.45 eq.) were added to 1,4-dioxane (15.0 mL), followed by purge with nitrogen for 3 min, and the mixture was reacted at 90° C. for 12 h. After the reaction was completed as monitored by TLC, the system was cooled to room temperature and filtered, and the filtrate was concentrated under reduced pressure to give a crude product (600.0 mg, used directly in the next step).

### Step 7: Synthesis of (R)-5-methyl-2-(4-(piperidin-3-ylamino)-5,6,7,8-tetrahydrophthalazin-1-yl)phenol

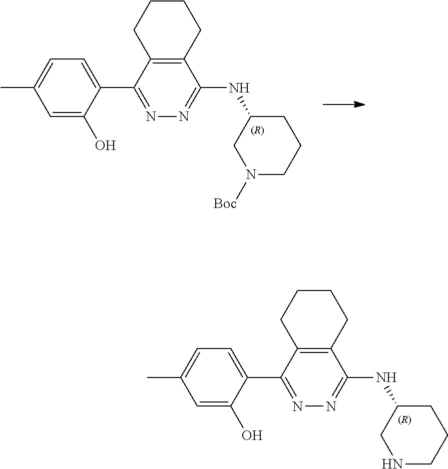

Crude tert-butyl (R)-3-((4-(2-hydroxy-4-methylphenyl)-5,6,7,8-tetrahydrophthalazin-1-yl)amino)piperidine-1-carboxylate (600.0 mg, 1.34 mmol, 1.0 eq.) was added to a mixture of dichloromethane (6.0 mL) and trifluoroacetic acid (6 mL), and the mixture was reacted at room temperature for 1 h. After the reaction was completed as monitored by TLC, water (50.0 mL) was added for back-extraction, and the pH of the aqueous phase was adjusted to 8-9, followed by extraction with dichloromethane (50.0 mL×2). The organic phase was dried over anhydrous sodium sulfate and filtered, and the filtrate was concentrated under reduced pressure to give the product (130.0 mg, two-step yield: 28.6%).

### Step 8: Synthesis of (R)-5-methyl-2-(4-((1-methylpiperidin-3-yl)amino)-5,6,7,8-tetrahydrophthalazin-1-yl)phenol

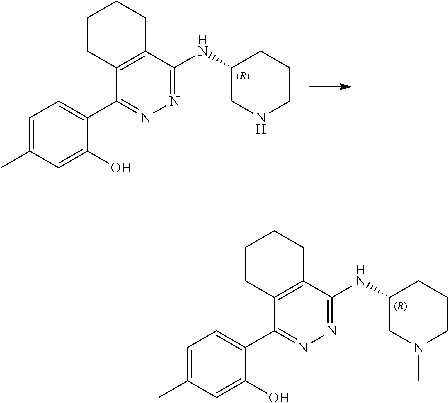

(R)-5-methyl-2-(4-(piperidin-3-ylamino)-5,6,7,8-tetrahydrophthalazin-1-yl)phenol (130.0 mg, 0.38 mmol, 1.0 eq.) was added to methanol (3.0 mL), and an aqueous formaldehyde solution (37%) (34.2 mg, 0.42 mmol, 1.1 eq.) was added. The mixture was stirred at room temperature for 1 h, followed by addition of sodium cyanoborohydride (31.0 mg, 0.49 mmol, 1.3 eq.), and the resulting mixture was reacted at room temperature for 2 h. After the reaction was completed as monitored by TLC, the system was concentrated under reduced pressure, and a saturated aqueous sodium bicarbonate solution (100.0 mL) was added, with stirring for 0.5 h, followed by extraction with dichloromethane (100.0 mL). The organic phase was dried over anhydrous sodium sulfate and filtered, the filtrate was concentrated under reduced pressure, and the crude product was purified by preparative thin-layer chromatography (dichloromethane:methanol=8:1) to give the product (30.0 mg, yield: 22.4%).

¹HNMR (300 MHz, DMSO-d6) δ (ppm): 9.63 (s, 1H), 6.98-7.00 (m, 1H), 6.67-6.72 (m, 2H), 5.71 (s, 1H), 4.37 (s, 1H), 2.89-3.20 (m, 3H), 2.37-2.45 (m, 6H), 2.27 (s, 3H), 1.59-1.85 (m, 7H), 1.24 (s, 3H).

Molecular formula: C₂₁H₂₈N₄O Molecular weight: 352.48 LC-MS (Pos, m/z)=353.26 [M+H]⁺.

### Example 57: Synthesis of Compound (R)-5-methyl-2-(4-((1-methylpiperidin-3-yl)amino)-6,7-dihydro-5H-cyclopenta[d]pyridazin-1-yl)phenol (Compound 3a)

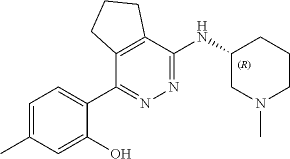

### Step 1: Synthesis of 2-(2-methoxy-4-methylbenzoyl)cyclopent-1-ene-1-carboxylic acid

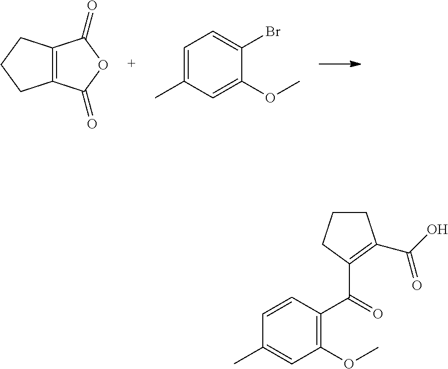

1-Bromo-2-methoxy-4-methylbenzene (7.27 g, 36.2 mmol, 1.0 eq.) was added to dry tetrahydrofuran (30.0 mL), and the mixture was cooled to −65° C. in nitrogen atmosphere, followed by dropwise and slow addition of a 2.5 mol/L solution of n-butyllithium in n-hexane (14.5 mL, 36.2 mmol, 1.0 eq.). After the reaction was carried out for 1 h, the reaction solution was added dropwise and slowly to a solution of 5,6-dihydro-1H-cyclopenta[c]furan-1,3(4H)-dione (5.0 g, 36.2 mmol, 1.0 eq.) in tetrahydrofuran (20.0 mL) at −65° C., and the solution was warmed to room temperature for 12 h. After the reaction was completed as monitored by TLC, a 2 mol/L aqueous HCl solution was added to the system with stirring for 0.5 h, followed by extraction with ethyl acetate (200.0 mL). The organic phase was dried over anhydrous sodium sulfate and filtered, and the filtrate was concentrated under reduced pressure to give the crude product (7.0 g) which was used directly in the next step.

### Step 2: Synthesis of methyl 2-(2-methoxy-4-methylbenzoyl)cyclopent-1-ene-1-carboxylate

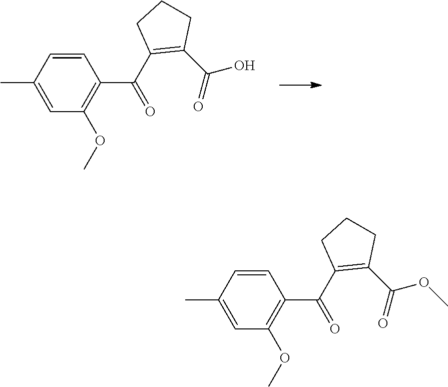

2-(2-Methoxy-4-methylbenzoyl)cyclopent-1-ene-1-carboxylic acid (7.0 g, 26.9 mmol, 1.0 eq.), potassium carbonate (5.57 g, 40.35 mmol, 1.5 eq.) and iodomethane (4.97 g, 35.0 mmol, 1.3 eq.) were added to N,N-dimethylformamide (20.0 mL), and the mixture was reacted at room temperature for 12 h. After the reaction was completed as monitored by TLC, ethyl acetate (200.0 mL) was added to the system, followed by washing with water (100.0 mL×4). The organic phase was dried over anhydrous sodium sulfate and filtered, the filtrate was concentrated under reduced pressure, and the crude product was purified by silica gel column chromatography (specification of silica gel: 100-200 mesh, petroleum ether:ethyl acetate=20:1 to 2:1) to give the product (5.0 g, two-step yield: 68.4%).

### Step 3: Synthesis of 4-(2-methoxy-4-methylphenyl)-6,7-dihydro-5H-cyclopenta[d]pyridazin-1-ol

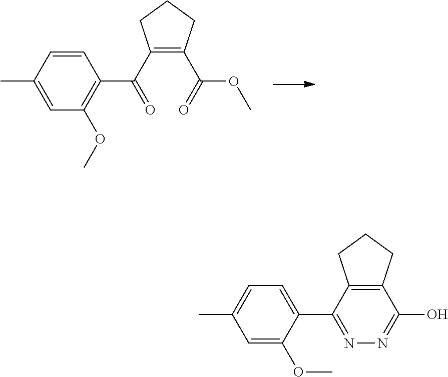

Methyl 2-(2-methoxy-4-methylbenzoyl)cyclopent-1-ene-1-carboxylate (4.75 g, 17.31 mmol, 1.0 eq.) was added to ethanol (50.0 mL), followed by addition of hydrazine hydrate (5.2 g, 103.89 mmol, 6.0 eq.), and the mixture was reacted at room temperature for 12 h and then reacted at 80° C. for 8 h. After the reaction was completed as monitored by TLC, the system was concentrated under reduced pressure, and the crude product was purified by silica gel column chromatography (specification of silica gel: 100-200 mesh, dichloromethane:methanol=20:1 to 10:1) to give the product (3.2 g, yield: 72.2%).

### Step 4: Synthesis of 1-chloro-4-(2-methoxy-4-methylphenyl)-6,7-dihydro-5H-cyclopenta[d]pyridazine

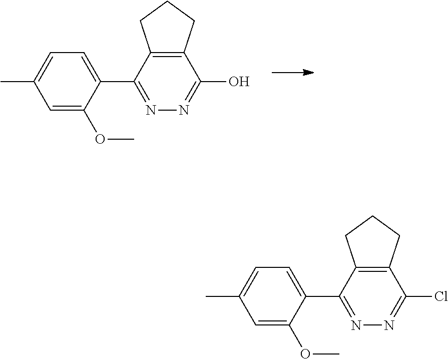 [SMILES: COc1cc(C)ccc1-c1nnc(O)c2c1CCC2 ; COc1cc(C)ccc1-c1nnc(Cl)c2c1CCC2]

4-(2-Methoxy-4-methylphenyl)-6,7-dihydro-5H-cyclopenta[d]pyridazin-1-ol (3.2 g, 12.48 mmol, 1.0 eq.) and phosphorus oxychloride (11.4 g, 74.88 mmol, 6.0 eq.) were added to acetonitrile (20.0 mL), and the mixture was stirred at 80° C. for 2 h. After the reaction was completed as monitored by TLC, the reaction solution was cooled to room temperature, a saturated aqueous sodium bicarbonate solution was added to carefully quench the reaction, and ethyl acetate (100.0 mL) was added to the system for extraction. The organic phase was dried over anhydrous sodium sulfate and filtered, and the filtrate was concentrated under reduced pressure to give the product (2.8 g, yield: 81.8%).

### Step 5: Synthesis of tert-butyl (R)-3-((4-(2-methoxy-4-methylphenyl)-6,7-dihydro-5H-cyclopenta[d]pyridazin-1-yl)amino)piperidine-1-carboxylate

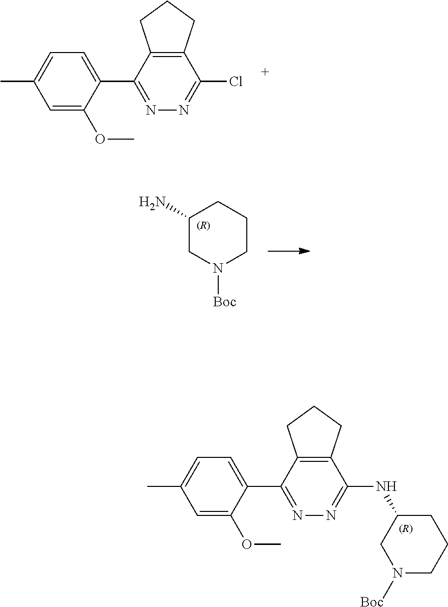

1-Chloro-4-(2-methoxy-4-methylphenyl)-6,7-dihydro-5H-cyclopenta[d]pyridazine (2.2 g, 8.0 mmol, 1.0 eq.) was added to 1,4-dioxane (20.0 mL), then tert-butyl (R)-3-aminopiperidine-1-carboxylate (3.2 g, 16.0 mmol, 2.0 eq.), cesium carbonate (7.8 g, 24.0 mmol, 3.0 eq.), Pd₂(dba)₃ (732.5 mg, 0.80 mmol, 0.1 eq.) and 1,1′-binaphthyl-2,2′-bisdiphenylphosphine (996.2 mg, 1.6 mmol, 0.2 eq.) were added successively, and the resulting mixture was reacted at 90° C. for 12 h in nitrogen atmosphere. After the reaction was completed as monitored by TLC, the system was cooled to room temperature and filtered, the filtrate was concentrated under reduced pressure, and the crude product was purified by silica gel column chromatography (specification of silica gel: 100-200 mesh, petroleum ether:ethyl acetate=10:1 to 1:1) to give the product (2.8 g, yield: 80%).

### Step 6: Synthesis of (R)-4-(2-methoxy-4-methylphenyl)-N-(piperidin-3-yl)-6,7-dihydro-5H-cyclopenta[d]pyridazin-1-amine

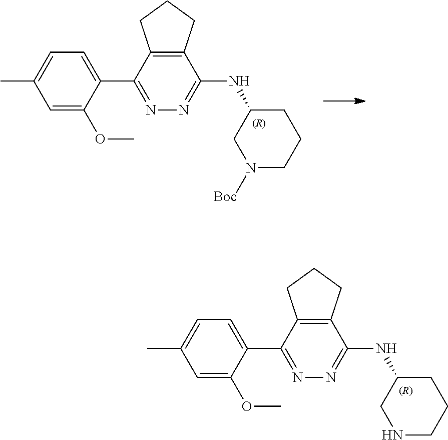

Tert-butyl (R)-3-((4-(2-methoxy-4-methylphenyl)-6,7-dihydro-5H-cyclopenta[d]pyridazin-1-yl)amino)piperidine-1-carboxylate (2.8 g, 6.38 mmol, 1.0 eq.) and trifluoroacetic acid (10.0 mL) were added to dichloromethane (10.0 mL), and the mixture was reacted at room temperature for 2 h. After the reaction was completed as monitored by TLC, a saturated aqueous sodium carbonate solution was added to the system to adjust the pH to 8-9, followed by extraction with dichloromethane (100.0 mL×2). The organic phase was dried over anhydrous sodium sulfate and filtered, and the filtrate was concentrated under reduced pressure to give the product (2.0 g, yield: 93.0%).

### Step 7: Synthesis of (R)-4-(2-methoxy-4-methylphenyl)-N-(1-methylpiperidin-3-yl)-6,7-dihydro-5H-cyclopenta[d]pyridazin-1-amine

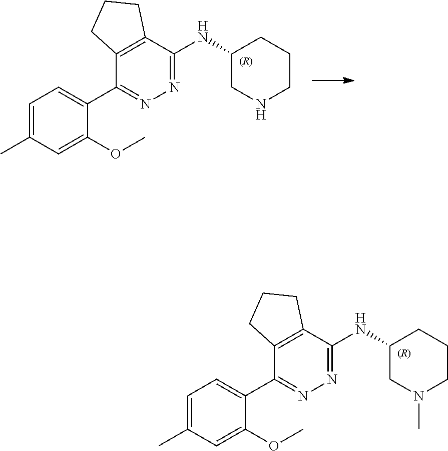

(R)-4-(2-methoxy-4-methylphenyl)-N-(piperidin-3-yl)-6,7-dihydro-5H-cyclopenta[d]pyridazin-1-amine (500.0 mg, 1.47 mmol, 1.0 eq.) and an aqueous formaldehyde solution (37%) (143.8 mg, 1.77 mmol, 1.2 eq.) were added to methanol (5.0 mL), and after stirring at room temperature for 1 h, sodium cyanoborohydride (129.3 mg, 2.05 mmol, 1.4 eq.) was added, and the resulting mixture was reacted at room temperature for 1 h. After the reaction was completed as monitored by TLC, the system was concentrated under reduced pressure, and a saturated aqueous sodium bicarbonate solution (100.0 mL) was added, with stirring for 0.5 h, followed by extraction with dichloromethane (100.0 mL). The organic phase was dried over anhydrous sodium sulfate and filtered, the filtrate was concentrated under reduced pressure, and the crude product was purified by silica gel column chromatography (specification of silica gel: 100-200 mesh, dichloromethane:methanol=50:1 to 10:1) to give the product (370.0 mg, yield: 71.4%).

### Step 8: Synthesis of (R)-5-methyl-2-(4-((1-methylpiperidin-3-yl)amino)-6,7-dihydro-5H-cyclopenta[d]pyridazin-1-yl)phenol

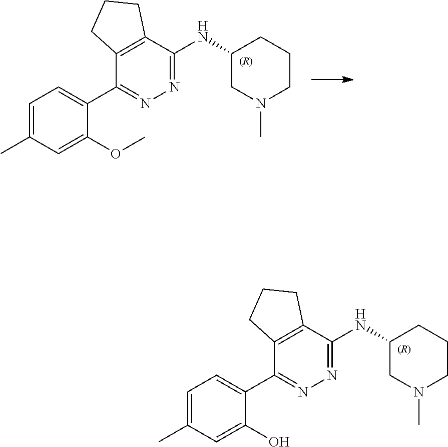

(R)-4-(2-methoxy-4-methylphenyl)-N-(1-methylpiperidin-3-yl)-6,7-dihydro-5H-cyclopenta[d]pyridazin-1-amine (370.0 mg, 1.04 mmol, 1.0 eq.) was added to dichloromethane (50.0 mL), the mixture was cooled to −50° C., followed by dropwise and slow addition of boron tribromide (788.9 mg, 3.14 mmol, 3.0 eq.), and the reaction solution was warmed to room temperature and stirred for 12 h. After the reaction was completed as monitored by TLC, methanol was added to the system to quench the reaction, followed by concentration under reduced pressure, and a saturated aqueous sodium bicarbonate solution was added to adjust the pH to 7-8, followed by extraction with dichloromethane (100.0 mL). The organic phase was dried over anhydrous sodium sulfate and filtered, the filtrate was concentrated under reduced pressure, and the crude product was purified by preparative thin-layer chromatography (dichloromethane:methanol=8:1) to give the product (165.0 mg, yield: 46.8%).

¹HNMR (300 MHz, DMSO-d6) δ (ppm): 7.48-7.51 (d, J=9 Hz, 1H), 6.91 (s, 1H), 6.70-6.73 (m, 1H), 4.70 (s, 1H), 3.26-3.31 (m, 2H), 2.86-2.98 (m, 4H), 2.71-2.74 (m, 1H), 2.48 (s, 3H), 2.40-2.42 (m, 1H), 2.35 (s, 3H), 2.17-2.26 (m, 2H), 1.69-2.06 (m, 4H).

Molecular formula: C₂₀H₂₆N₄O Molecular weight: 338.46 LC-MS (Pos, m/z)=339.17 [M+H]⁺.

### Example 58: Synthesis of Compound (R)-5-cyclopropyl-2-(4-((1-methylpiperidin-3-yl)amino)-5,6,7,8-tetrahydrophthalazin-1-yl)phenol (Compound 5a)

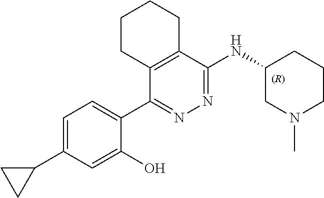

### Step 1: Synthesis of 1-chloro-4-(4-cyclopropyl-2-methoxyphenyl)-5,6,7,8-tetrahydrophthalazine

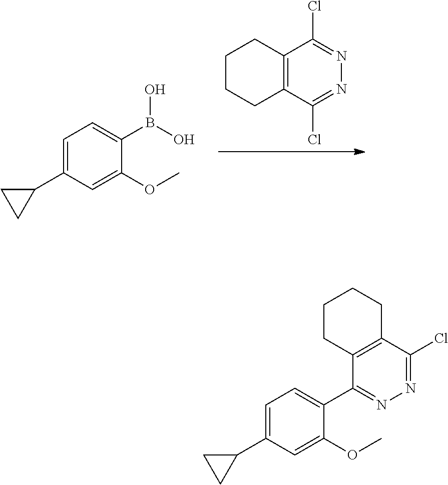 [SMILES: COc1cc(C2CC2)ccc1B(O)O ; Clc1nnc(Cl)c2c1CCCC2 ; COc1cc(C2CC2)ccc1-c1nnc(Cl)c2c1CCCC2]

1,4-Dichloro-5,6,7,8-tetrahydrophthalazine (613.3 mg, 3.02 mmol, 1.0 eq.), (4-cyclopropyl-2-methoxyphenyl)boronic acid (580.0 mg, 3.02 mmol, 1.0 eq.), sodium bicarbonate (507.5 mg, 6.04 mmol, 2.0 eq.) and Pd(pph₃)₄ (348.9 mg, 0.30 mmol, 0.1 eq.) were added to 1,4-dioxane (10.0 mL) and water (5.0 mL), and the mixture was reacted at 110° C. for 2 h in nitrogen atmosphere. After the reaction was completed as monitored by TLC, the system was cooled to room temperature, followed by addition of ethyl acetate (100.0 mL) and washing with water (50.0 mL). The organic phase was dried over anhydrous sodium sulfate and filtered, the filtrate was concentrated under reduced pressure, and the crude product was purified by silica gel column chromatography (specification of silica gel: 100-200 mesh, petroleum ether:ethyl acetate=10:1 to 4:1) to give the product (500.0 mg, yield: 52.6%).

### Step 2: Synthesis of tert-butyl (R)-3-((4-(4-cyclopropyl-2-methoxyphenyl)-5,6,7,8-tetrahydrophthalazin-1-yl)amino)piperidine-1-carboxylate

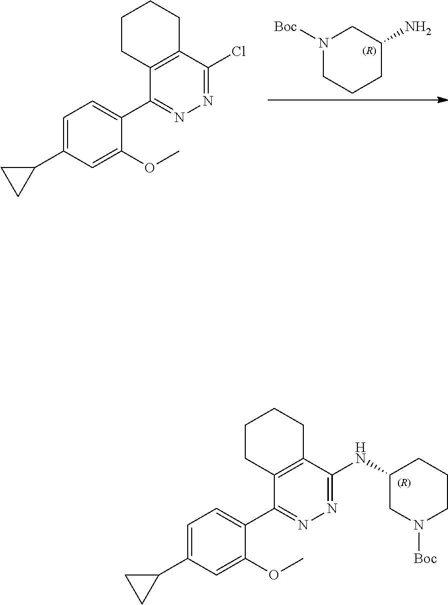

1-Chloro-4-(4-cyclopropyl-2-methoxyphenyl)-5,6,7,8-tetrahydrophthalazine (500.0 mg, 1.58 mmol, 1.0 eq.), tert-butyl (R)-3-aminopiperidine-1-carboxylate (636.1 mg, 3.17 mmol, 2.0 eq.), cesium carbonate (1.54 g, 4.74 mmol, 3.0 eq.), Pd₂(dba)₃ (144.6 mg, 0.15 mmol, 0.1 eq.) and BINAP (196.7 mg, 0.31 mmol, 0.2 eq.) were added to 1,4-dioxane (15.0 mL), and the mixture was reacted at 90° C. for 12 h in nitrogen atmosphere. After the reaction was completed as monitored by TLC, the reaction solution was cooled, followed by addition of ethyl acetate (200.0 mL) and washing with water (100.0 mL). The organic phase was dried over anhydrous sodium sulfate and filtered, the filtrate was concentrated under reduced pressure, and the crude product was purified by silica gel column chromatography (specification of silica gel: 100-200 mesh, dichloromethane:methanol=100:1 to 80:1) to give the product (680.0 mg, yield: 89.9%).

### Step 3: Synthesis of (R)-4-(4-cyclopropyl-2-methoxyphenyl)-N-(piperidin-3-yl)-5,6,7,8-tetrahydrophthalazin-1-amine

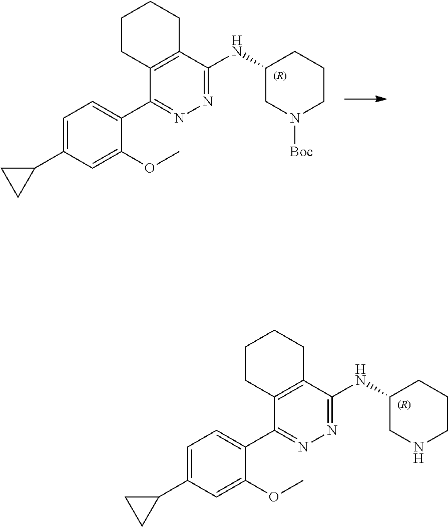

Tert-butyl (R)-3-((4-(4-cyclopropyl-2-methoxyphenyl)-5,6,7,8-tetrahydrophthalazin-1-yl)amino)piperidine-1-carboxylate (680.0 mg, 1.42 mmol, 1.0 eq.) and trifluoroacetic acid (5.0 mL) were added to dichloromethane (10.0 mL), and the mixture was stirred at room temperature for 2 h. After the reaction was completed as monitored by TLC, a saturated aqueous sodium bicarbonate solution was added to the system to adjust the pH to 7-8, followed by extraction with dichloromethane (100.0 mL). The organic phase was dried over anhydrous sodium sulfate and filtered, and the filtrate was concentrated under reduced pressure to give the product (530.0 mg, yield: 98.6%).

### Step 4: Synthesis of (R)-4-(4-cyclopropyl-2-methoxyphenyl)-N-(1-methylpiperidin-3-yl)-5,6,7,8-tetrahydrophthalazin-1-amine

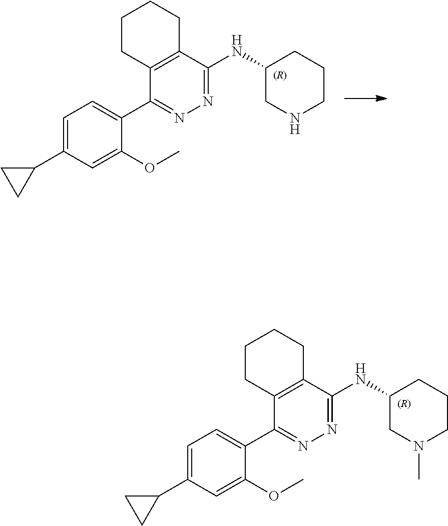

(R)-4-(4-cyclopropyl-2-methoxyphenyl)-N-(piperidin-3-yl)-5,6,7,8-tetrahydrophthalazin-1-amine (530.0 mg, 1.40 mmol, 1.0 eq.) and an aqueous formaldehyde solution (37%) (136.3 mg, 1.68 mmol, 1.2 eq.) were added to methanol (10.0 mL), the mixture was stirred at room temperature for 0.5 h, followed by addition of sodium cyanoborohydride (123.1 mg, 1.96 mmol, 1.4 eq.), and the reaction solution was then reacted at room temperature for 2 h. After the reaction was completed as monitored by TLC, the system was concentrated under reduced pressure, followed by addition of a saturated aqueous sodium bicarbonate solution (100.0 mL). The system was stirred for 30 min and extracted with dichloromethane (50.0 mL). The organic phase was dried over anhydrous sodium sulfate and filtered, and the filtrate was concentrated under reduced pressure. The crude product was purified by silica gel column chromatography (specification of silica gel: 100-200 mesh, dichloromethane:methanol=50:1 to 10:1) to give the product (412.1 mg, yield: 75.0%).

### Step 5: Synthesis of (R)-5-cyclopropyl-2-(4-((1-methylpiperidin-3-yl)amino)-5,6,7,8-tetrahydrophthalazin-1-yl)phenol

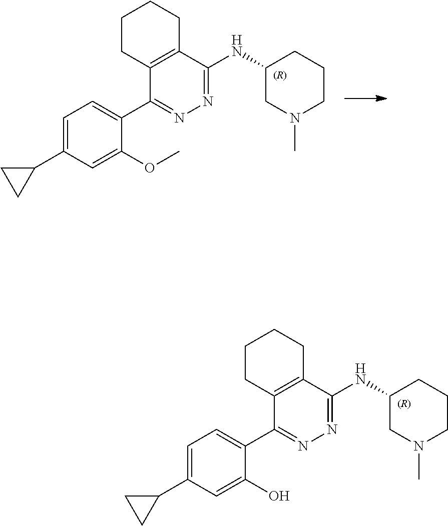

(R)-4-(4-cyclopropyl-2-methoxyphenyl)-N-(1-methylpiperidin-3-yl)-5,6,7,8-tetrahydrophthalazin-1-amine (400.0 mg, 1.01 mmol, 1.0 eq.) was added to dichloromethane (4.0 mL), boron tribromide (765.8 mg, 3.03 mmol, 3.0 eq.) was added dropwise under an ice bath, and the reaction solution was warmed to room temperature and stirred for 4 h. After the reaction was completed as monitored by TLC, methanol (5.0 mL) was added under an ice bath to quench the reaction, followed by concentration under reduced pressure, addition of dichloromethane (100.0 mL) and back extraction with water (50.0 mL). A saturated aqueous sodium bicarbonate solution was added to the aqueous phase to adjust the pH to 7-8, followed by extraction with dichloromethane (100.0 mL). The organic phase was dried over anhydrous sodium sulfate and filtered, the filtrate was concentrated under reduced pressure, and the crude product was purified by preparative thin-layer chromatography to give the product (170.0 mg, yield: 44.4%).

¹HNMR (300 MHz, DMSO-d6) δ (ppm): 9.66 (s, 1H), 7.08-6.98 (m, 1H), 6.87-6.56 (m, 2H), 5.53-5.50 (m, 1H), 4.26 (s, 1H), 2.95-2.93 (m, 1H), 2.65-2.62 (m, 1H), 2.35-2.33 (m, 4H), 2.23 (s, 3H), 2.04-2.01 (m, 2H), 1.87-1.69 (m, 6H), 1.58-1.47 (m, 4H), 0.96-0.93 (m, 2H), 0.86-0.83 (m, 1H), 0.66-0.63 (m, 1H).

Molecular formula: C₂₃H₃₀N₄O Precise molecular weight: 378.24 LC-MS (m/z)=379.17 [M+H]⁺.

### Example 59: Synthesis of Compound (R)-5-methyl-2-(4-((1-methylpiperidin-3-yl)amino)-5,6,7,8-tetrahydro-5,8-ethanophthalazin-1-yl)phenol (Compound 6a)

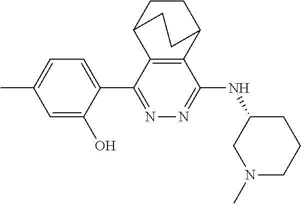 [SMILES: Cc1ccc(-c2nnc(N[C@@H]3CCCN(C)C3)c3c2C2CCC3CC2)c(O)c1]

### Step 1: Synthesis of tert-butyl (R)-3-((4-chloro-5,6,7,8-tetrahydro-5,8-ethanophthalazin-1-yl)amino)piperidine-1-carboxylate

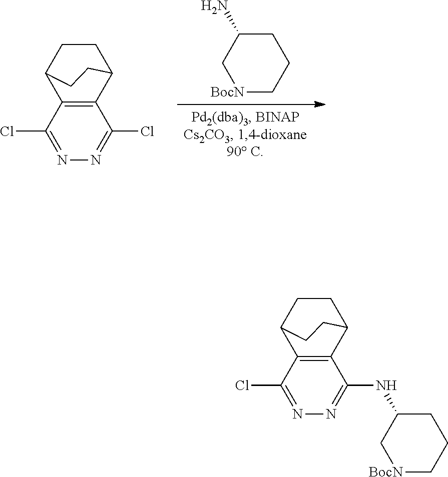

1,4-Dichloro-5,6,7,8-tetrahydro-5,8-ethanophthalazine (4.50 g, 19.6 mmol, 1.0 eq.), tert-butyl (R)-3-aminopiperidine-1-carboxylate (4.32 g, 21.6 mmol, 1.1 eq.), Pd₂(dba)₃ (1.79 g, 1.96 mmol, 0.1 eq.), BINAP (2.44 g, 3.92 mmol, 0.2 eq.) and Cs₂CO₃ (12.8 g, 39.2 mmol, 2.0 eq.) were added successively to 1,4-dioxane (100 mL), and the mixture was heated to 90° C. and reacted for 20 h in nitrogen atmosphere. The system was cooled to room temperature, and water was added to quench the reaction, followed by extraction with EA (100 mL×2). The organic phase was dried over anhydrous sodium sulfate and filtered, the filtrate was concentrated under reduced pressure, and the crude product was purified by silica gel column chromatography (PE:EA=10:1 to 1:1) to give the product (5.90 g, yield: 76.4%).

### Step 2: Synthesis of (R)-4-chloro-N-(piperidin-3-yl)-5,6,7,8-tetrahydro-5,8-ethanophthalazin-1-amine

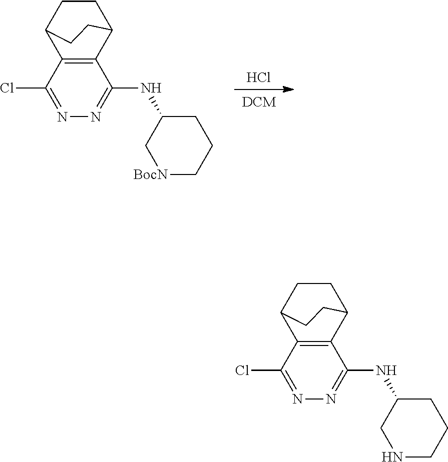

Tert-butyl (R)-3-((4-chloro-5,6,7,8-tetrahydro-5,8-ethanophthalazin-1-yl)amino)piperidine-1-carboxylate (5.90 g, 15.0 mmol, 1.0 eq.) was dissolved in DCM (50 mL), then a solution of hydrogen chloride in 1,4-dioxane (4 mol/L, 37.5 mL, 150 mmol, 10.0 eq.) was added dropwise, and the reaction solution was stirred at room temperature for 4 h. Then water (80 mL) was added, followed by liquid separation. The organic phase was removed, and a saturated aqueous NaHCO₃ solution was added to the aqueous phase to adjust the pH to 8, followed by extraction with DCM (50 mL×5). The organic phase was dried over anhydrous sodium sulfate and filtered, and the filtrate was concentrated under reduced pressure to give the product (3.60 g, yield: 81.9%).

### Step 3: Synthesis of (R)-4-chloro-N-(1-methylpiperidin-3-yl)-5,6,7,8-tetrahydro-5,8-ethanophthalazin-1-amine

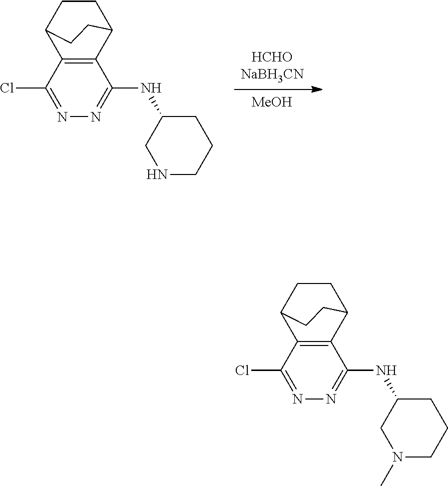 [SMILES: "MeOH" ; Clc1nnc(N[C@@H]2CCCNC2)c2c1C1CCC2CC1 ; "HCHONaBH3CN" ; CN1CCC[C@@H](Nc2nnc(Cl)c3c2C2CCC3CC2)C1]

(R)-4-chloro-N-(piperidin-3-yl)-5,6,7,8-tetrahydro-5,8-ethanophthalazin-1-amine (3.60 g, 12.3 mmol, 1.0 eq.) was dissolved in MeOH (40 mL), an aqueous formaldehyde solution (37%, 1.20 g, 14.8 mmol, 1.2 eq.) was added, and the reaction solution was stirred at room temperature for 30 min. NaBH₃CN (930 mg, 14.8 mmol, 1.2 eq.) was then added, and the reaction solution was reacted at room temperature for 1 h. MeOH was removed by concentration, the system was then dissolved in DCM, and a saturated aqueous NaHCO₃ solution was added for washing. The aqueous phase was extracted with DCM (50 mL×2), the organic phases were combined, dried over anhydrous sodium sulfate, and filtered, and the filtrate was concentrated under reduced pressure. The crude product was purified by silica gel column chromatography (DCM:MeOH=50:1 to 20:1) to give the product (3.20 g, yield: 84.8%).

### Step 4: Synthesis of (R)-4-(2-methoxy-4-methylphenyl)-N-(1-methylpiperidin-3-yl)-5,6,7,8-tetrahydro-5,8-ethanophthalazin-1-amine

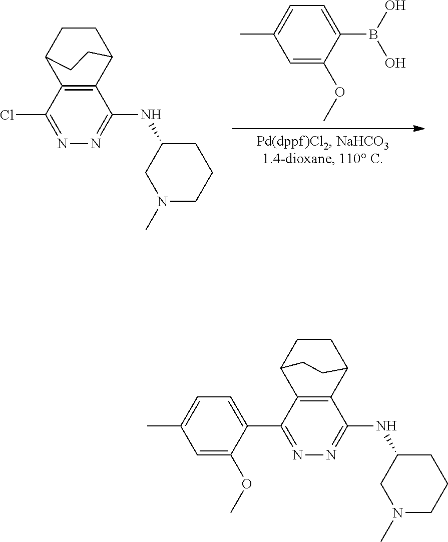

(2-Methoxy-4-methylphenyl)boronic acid (200 mg, 1.20 mmol, 1.0 eq.), (R)-4-chloro-N-(1-methylpiperidin-3-yl)-5,6,7,8-tetrahydro-5,8-ethanophthalazin-1-amine (370 mg, 1.20 mmol, 1.0 eq.), Pd(dppf)Cl₂ (43.9 mg, 0.060 mmol, 0.05 eq.) and NaHCO₃ (202 mg, 2.40 mmol, 2.0 eq.) were added successively to 1,4-dioxane (6 mL), then H₂O (3 mL) was added, and the reaction solution was heated to 110° C. for 2 h in nitrogen atmosphere. The system was cooled to room temperature, and water (30 mL) was added to quench the reaction, followed by extraction with DCM (30 mL×2). The organic phase was dried over anhydrous sodium sulfate and filtered, the filtrate was concentrated under reduced pressure, and the crude product was purified by silica gel column chromatography (DCM:MeOH=50:1 to 10:1) to give the product (320 mg, yield: 67.7%).

### Step 5: Synthesis of (R)-5-methyl-2-(4-((1-methylpiperidin-3-yl)amino)-5,6,7,8-tetrahydro-5,8-ethanophthalazin-1-yl)phenol

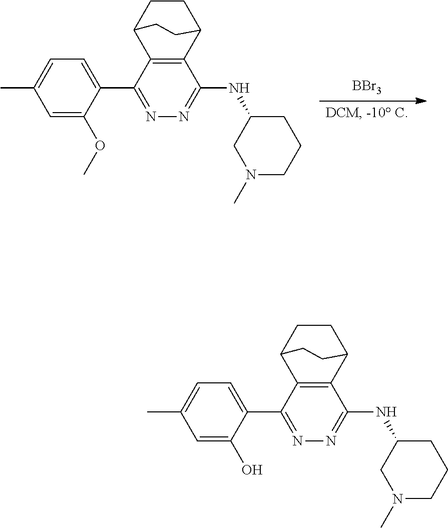 [SMILES: COc1cc(C)ccc1-c1nnc(N[C@@H]2CCCN(C)C2)c2c1C1CCC2CC1 ; "BBr3" ; "DCM, -10° C." ; Cc1ccc(-c2nnc(N[C@@H]3CCCN(C)C3)c3c2C2CCC3CC2)c(O)c1]

(R)-4-(2-methoxy-4-methylphenyl)-N-(1-methylpiperidin-3-yl)-5,6,7,8-tetrahydro-5,8-ethanophthalazin-1-amine (320 mg, 0.815 mmol, 1.0 eq.) was dissolved in DCM (10 mL), the reaction solution was cooled to −10° C., followed by dropwise addition of BBr₃ (1.02 g, 4.07 mmol, 5.0 eq.), and the solution was naturally warmed to room temperature and reacted for 16 h. MeOH (10 mL) was added to quench the reaction, a saturated aqueous NaHCO₃ solution was added to adjust the pH to 8, and DCM (30 mL×3) was added for extraction. The organic phase was dried over anhydrous sodium sulfate and filtered, and the filtrate was concentrated under reduced pressure. The crude product was purified by silica gel column chromatography (DCM:MeOH=50:1 to 10:1) to give the product (160 mg, yield: 51.9%).

¹HNMR (400 MHz, DMSO-d6) δ (ppm): 9.90 (s, 1H), 7.10 (d, J=7.6 Hz, 1H), 6.76 (s, 1H), 6.71 (d, J=7.7 Hz, 1H), 6.41 (s, 1H), 4.43 (s, 1H), 3.45 (s, 1H), 3.06 (s, 1H), 2.83 (s, 1H), 2.59 (s, 4H), 2.28 (s, 3H), 1.92 (d, J=3.3 Hz, 2H), 1.74-1.66 (m, 7H), 1.74-1.24 (s, 5H).

Molecular formula: C₂₃H₃₀N₄O Molecular weight: 378.24 LC-MS (m/z)=379.19 [M+H]⁺.

### Example 60: Synthesis of Compound (R)-3-hydroxy-4-(4-((1-methylpiperidin-3-yl)amino)-5,6,7,8-tetrahydro-5,8-ethanophthalazin-1-yl)benzonitrile (Compound 7a)

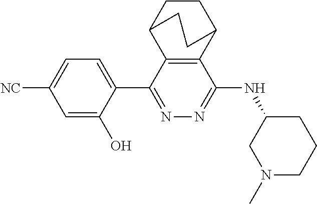 [SMILES: CN1CCC[C@@H](Nc2nnc(-c3ccc(C#N)cc3O)c3c2C2CCC3CC2)C1]

### Step 1: Synthesis of (R)-3-(ethoxymethoxy)-4-(4-((1-methylpiperidin-3-yl)amino)-5,6,7,8-tetrahydro-5,8-ethanophthalazin-1-yl)benzonitrile

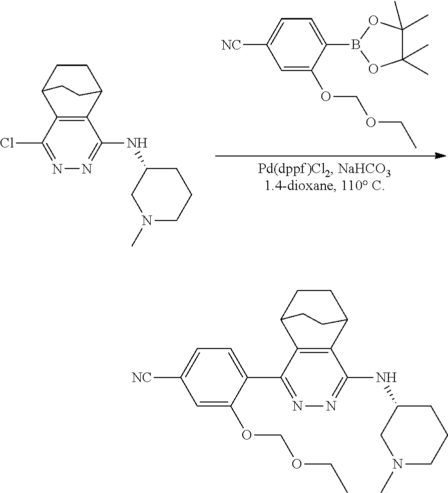 [SMILES: CN1CCC[C@@H](Nc2nnc(Cl)c3c2C2CCC3CC2)C1 ; CCOCOc1cc(C#N)ccc1B1OC(C)(C)C(C)(C)O1 ; "Pd(dppf)Cl2, NaHCO31.4-dioxane, 110° C." ; CCOCOc1cc(C#N)ccc1-c1nnc(N[C@@H]2CCCN(C)C2)c2c1C1CCC2CC1]

3-(Ethoxymethoxy)-4-(4,4,5,5-tetramethyl-1,3,2-dioxaborolan-2-yl)benzonitrile (300 mg, 0.990 mmol, 1.0 eq.), (R)-4-chloro-N-(1-methylpiperidin-3-yl)-5,6,7,8-tetrahydro-5,8-ethanophthalazin-1-amine (304 mg, 0.990 mmol, 1.0 eq.), Pd(dppf)Cl₂ (36.2 mg, 0.0495 mmol, 0.05 eq.) and NaHCO₃ (166 mg, 1.98 mmol, 2.0 eq.) were added successively to 1,4-dioxane (6 mL) and H₂O (3 mL), and the mixture was heated to 110° C. for 2 h in nitrogen atmosphere. The system was cooled to room temperature, and water (30 mL) was added to quench the reaction, followed by extraction with DCM (30 mL×2). The organic phase was dried over anhydrous sodium sulfate and filtered, the filtrate was concentrated under reduced pressure, and the crude product was purified by silica gel column chromatography (DCM:MeOH=50:1 to 10:1) to give the product (265 mg, yield: 59.8%).

### Step 2: Synthesis of (R)-3-hydroxy-4-(4-((1-methylpiperidin-3-yl)amino)-5,6,7,8-tetrahydro-5,8-ethanophthalazin-1-yl)benzonitrile

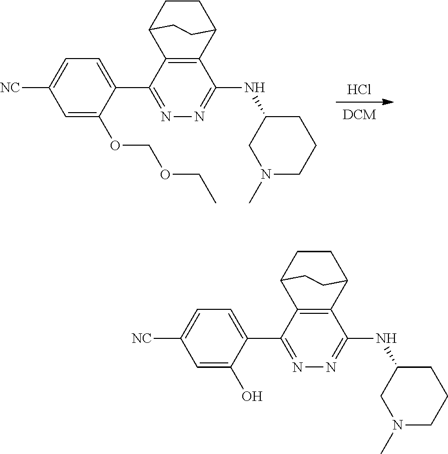

(R)-3-(ethoxymethoxy)-4-(4-((1-methylpiperidin-3-yl)amino)-5,6,7,8-tetrahydro-5,8-ethanophthalazin-1-yl)benzonitrile (265 mg, 0.592 mmol, 1.0 eq.) was dissolved in DCM (4 mL), then a solution of hydrogen chloride in 1,4-dioxane (4 mol/L, 0.74 mL, 2.96 mmol, 5.0 eq.) was added dropwise, and the reaction solution was stirred at room temperature for 1 h. Water (20 mL) was added to quench the reaction, followed by washing with DCM (20 mL×2), and a saturated aqueous NaHCO₃ solution was added to the aqueous phase to adjust the pH to 8, followed by extraction with DCM (20 mL×2). The organic phase was dried over anhydrous sodium sulfate and filtered, and the filtrate was concentrated under reduced pressure and slurried with EA to give the product (172 mg, yield: 74.6%).

¹HNMR (400 MHz, DMSO-d6) δ (ppm): 7.40 (d, J=7.5 Hz, 1H), 7.29 (s, 1H), 7.21 (s, 1H), 6.14 (s, 1H), 4.20 (s, 1H), 3.57 (s, 1H), 2.99 (s, 1H), 2.72 (s, 1H), 2.65 (s, 1H), 2.18 (s, 3H), 1.88-1.57 (m, 9H), 1.35-1.18 (m, 5H).

Molecular formula: C₂₃H₂₇N₅O Precise molecular weight: 389.22 LC-MS (m/z)=390.14 [M+H]⁺.

### Example 61: Synthesis of Compound (R)-2-(4-((1-methylpiperidin-3-yl)amino)-5,6,7,8-tetrahydrophthalazin-1-yl)-5-(trifluoromethyl)phenol (Compound 8a)

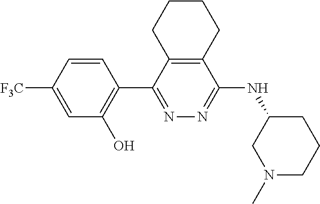 [SMILES: CN1CCC[C@@H](Nc2nnc(-c3ccc(C(F)(F)F)cc3O)c3c2CCCC3)C1]

### Step 1: Synthesis of 1-chloro-4-(2-(ethoxymethoxy)-4-(trifluoromethyl)phenyl)-5,6,7,8-tetrahydrophthalazine

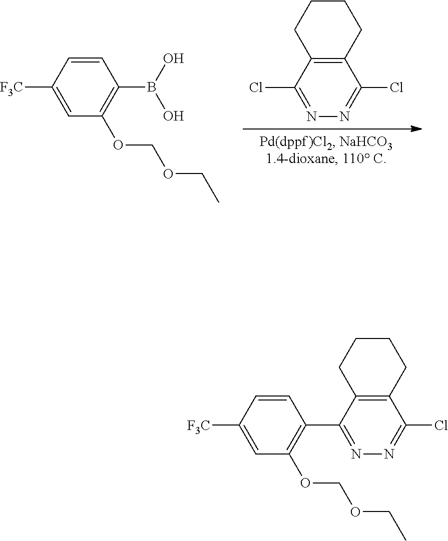

(2-(Ethoxymethoxy)-4-(trifluoromethyl)phenyl)boronic acid (1.00 g, 3.79 mmol, 1.0 eq.), 1,4-dichloro-5,6,7,8-tetrahydrophthalazine (769 mg, 3.79 mmol, 1.0 eq.), Pd(dppf)Cl₂ (139 mg, 0.190 mmol, 0.05 eq.) and NaHCO₃ (637 mg, 7.58 mmol, 2.0 eq.) were added successively to 1,4-dioxane (10 mL), then H₂O (5 mL) was added, and the reaction solution was heated to 110° C. and reacted for 3 h in nitrogen atmosphere. The system was cooled to room temperature, and water (40 mL) was added to quench the reaction, followed by extraction with EA (30 mL×2). The organic phase was dried over anhydrous sodium sulfate and filtered, the filtrate was concentrated under reduced pressure, and the crude product was purified by silica gel column chromatography (PE:EA=20:1 to 5:1) to give the product (890 mg, yield: 60.7%).

### Step 2: Synthesis of tert-butyl (R)-3-((4-(2-(ethoxymethoxy)-4-(trifluoromethyl)phenyl)-5,6,7,8-tetrahydrophthalazin-1-yl)amino)piperidine-1-carboxylate

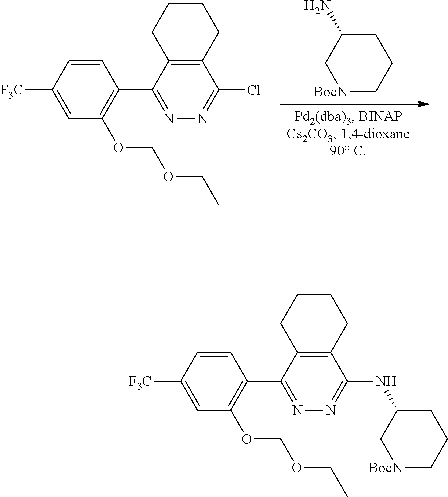

1-Chloro-4-(2-(ethoxymethoxy)-4-(trifluoromethyl)phenyl)-5,6,7,8-tetrahydrophthalazine (890 mg, 2.30 mmol, 1.0 eq.), tert-butyl (R)-3-aminopiperidine-1-carboxylate (691 mg, 2.45 mmol, 1.5 eq.), Pd₂(dba)₃ (211 mg, 0.230 mmol, 0.1 eq.), BINAP (286 mg, 0.460 mmol, 0.2 eq.) and Cs₂CO₃ (1.50 g, 4.60 mmol, 2.0 eq.) were added successively to 1,4-dioxane (20 mL), and the reaction solution was heated to 90° C. and reacted for 4 h in nitrogen atmosphere. The system was cooled to room temperature, and water (50 mL) was added to quench the reaction, followed by extraction with EA (40 mL×2). The organic phase was dried over anhydrous sodium sulfate and filtered, the filtrate was concentrated under reduced pressure, and the crude product was purified by silica gel column chromatography (DCM:MeOH=100:1 to 50:1) to give the product (1.10 g, yield: 86.8%).

### Step 3: Synthesis of (R)-2-(4-(piperidin-3-ylamino)-5,6,7,8-tetrahydrophthalazin-1-yl)-5-(trifluoromethyl)phenol

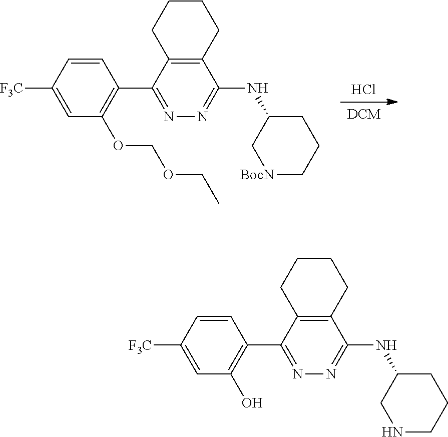

Tert-butyl (R)-3-((4-(2-(ethoxymethoxy)-4-(trifluoromethyl)phenyl)-5,6,7,8-tetrahydrophthalazin-1-yl)amino)piperidine-1-carboxylate (1.10 g, 2.00 mmol, 1.0 eq.) was dissolved in DCM (10 mL), then a solution of hydrogen chloride in 1,4-dioxane (4 mol/L, 5.0 mL, 20.0 mmol, 10.0 eq.) was added dropwise, and the reaction solution was stirred at room temperature for 1 h. Then water (30 mL) was added to quench the reaction, followed by washing with DCM (30 mL×2), and a saturated aqueous NaHCO₃ solution was added to the aqueous phase to adjust the pH to 8, followed by extraction with DCM (30 mL×2). The organic phase was dried over anhydrous sodium sulfate and filtered, and the filtrate was concentrated under reduced pressure to give the product (620 mg, yield: 79.1%).

### Step 4: Synthesis of (R)-2-(4-((1-methylpiperidin-3-yl)amino)-5,6,7,8-tetrahydrophthalazin-1-yl)-5-(trifluoromethyl)phenol

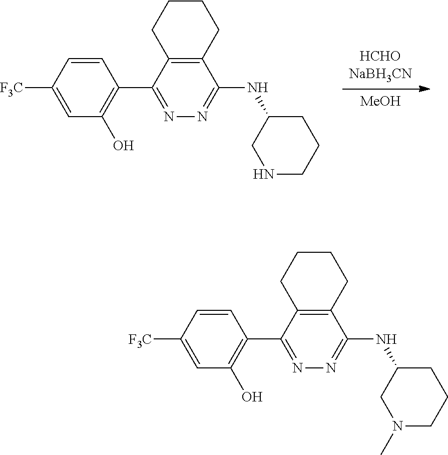 [SMILES: "MeOH" ; Oc1cc(C(F)(F)F)ccc1-c1nnc(N[C@@H]2CCCNC2)c2c1CCCC2 ; "HCHONaBH3CN" ; CN1CCC[C@@H](Nc2nnc(-c3ccc(C(F)(F)F)cc3O)c3c2CCCC3)C1]

(R)-2-(4-(piperidin-3-ylamino)-5,6,7,8-tetrahydrophthalazin-1-yl)-5-(trifluoromethyl)phenol (620 mg, 1.58 mmol, 1.0 eq.) was dissolved in MeOH (10 mL), then an aqueous formaldehyde solution (37%, 154 mg, 1.90 mmol, 1.2 eq.) was added, and the reaction solution was stirred at room temperature for 20 min. NaBH₃CN (119 mg, 1.90 mmol, 1.2 eq.) was then added, and the reaction solution was reacted at room temperature for 1 h. After concentration, the system was then dissolved in DCM, and a saturated aqueous NaHCO₃ solution was added for washing. The aqueous phase was extracted with DCM (20 mL×2), the organic phases were combined, dried over anhydrous sodium sulfate, and filtered, and the filtrate was concentrated under reduced pressure. The crude product was purified by silica gel column chromatography (DCM:MeOH=50:1 to 20:1) to give the product (410 mg, yield: 63.9%).

¹HNMR (400 MHz, DMSO-d6) δ (ppm): 10.43 (s, 1H), 7.34 (d, J=7.5 Hz, 1H), 7.21 (d, J=7.5 Hz, 2H), 5.63 (d, J=7.8 Hz, 1H), 4.30-4.24 (m, 1H), 2.93 (d, J=8.4 Hz, 1H), 2.61 (s, 1H), 2.38-2.35 (m, 2H), 2.32-2.30 (m, 2H), 2.21 (s, 3H), 1.99 (d, J=8.8 Hz, 2H), 1.83 (d, J=8.8 Hz, 1H), 1.76-1.69 (m, 3H), 1.61-1.54 (m, 3H), 1.48-1.43 (m, 1H).

Molecular formula: C₂₁H₂₅F₃N₄O Precise molecular weight: 406.20 LC-MS (m/z)=407.12 [M+H]⁺.

### Example 62: Synthesis of Compound (R)-3-hydroxy-4-(4-((1-(methylsulfonyl)piperidin-3-yl)amino)-5,6,7,8-tetrahydrophthalazin-1-yl)benzonitrile (Compound 13a)

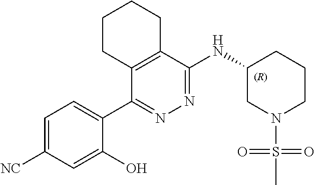

### Step 1: Synthesis of (R)-3-(ethoxymethoxy)-4-(4-((1-(methylsulfonyl)piperidin-3-yl)amino)-5,6,7,8-tetrahydrophthalazin-1-yl)benzonitrile

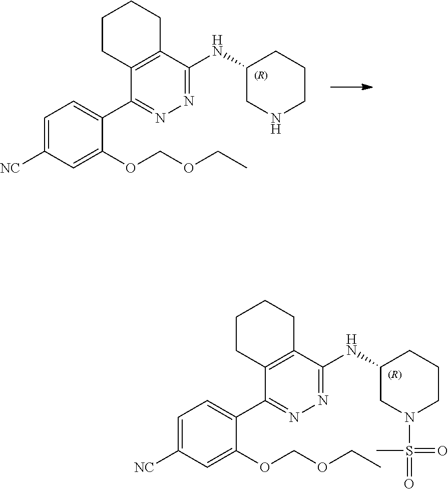

(R)-3-(ethoxymethoxy)-4-(4-(piperidin-3-ylamino)-5,6,7,8-tetrahydrophthalazin-1-yl)benzonitrile (317.0 mg, 0.77 mmol, 1.0 eq.), triethylamine (116.8 mg, 1.15 mmol, 1.5 eq.) and methanesulfonic anhydride (176.1 mg, 1.01 mmol, 1.3 eq.) were added to dichloromethane (10.0 mL), and the reaction solution was reacted at room temperature for 12 h. After the reaction was completed as monitored by TLC, a saturated aqueous ammonium chloride solution (50.0 mL) was added, then dichloromethane (50.0 mL) was added for extraction, and the organic phase was dried over anhydrous sodium sulfate and filtered. The filtrate was concentrated under reduced pressure to give the product (343.0 mg, yield: 91.9%).

### Step 2: Synthesis of (R)-3-hydroxy-4-(4-((1-(methylsulfonyl)piperidin-3-yl)amino)-5,6,7,8-tetrahydrophthalazin-1-yl)benzonitrile

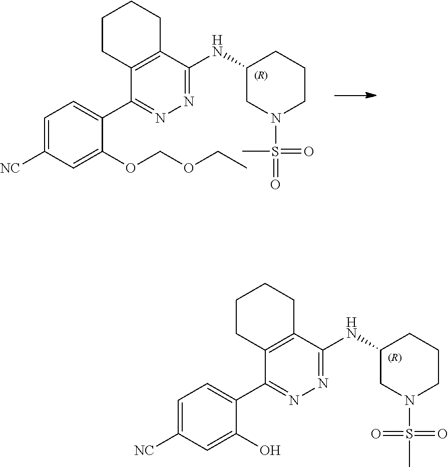

(R)-3-(ethoxymethoxy)-4-(4-((1-(methylsulfonyl)piperidin-3-yl)amino)-5,6,7,8-tetrahydrophthalazin-1-yl)benzonitrile (343.0 mg, 0.70 mmol, 1.0 eq.) and a solution of hydrogen chloride in 1,4-dioxane (4.0 mol/L, 3.0 mL) were added to dichloromethane (3.0 mL), and the reaction solution was reacted at room temperature for 2 h. After the reaction was completed as monitored by TLC, a saturated aqueous sodium bicarbonate solution was added to the system to adjust the pH to 7-8, followed by extraction with dichloromethane (50.0 mL). The organic phase was dried over anhydrous sodium sulfate and filtered, the filtrate was concentrated under reduced pressure, and the crude product was purified by preparative thin-layer chromatography to give the product (207.0 mg, yield: 69.2%).

¹HNMR (400 MHz, DMSO-d6) δ (ppm): 10.50 (s, 1H), 7.33 (s, 2H), 7.24 (s, 1H), 5.84-5.82 (d, J=4 Hz, 1H), 4.26-4.24 (m, 1H), 3.88-3.84 (m, 1H), 3.52-3.47 (m, 1H), 2.88 (s, 3H), 2.75-2.70 (m, 1H), 2.58-2.53 (m, 1H), 2.40-2.37 (m, 2H), 2.31-2.28 (m, 2H), 1.97-1.95 (m, 1H), 1.88-1.86 (m, 1H), 1.77-1.75 (m, 2H), 1.61-1.60 (m, 4H).

Molecular formula: C₂₁H₂₅N₅O₃S Precise molecular weight: 427.17 LC-MS (m/z)=428.1 [M+H]⁺.

### Example 63: Synthesis of (R)-3-hydroxy-4-(4-((1-methylpiperidin-3-yl)amino)-5,6,7,8-tetrahydrophthalazin-1-yl)benzonitrile (Compound 2a)

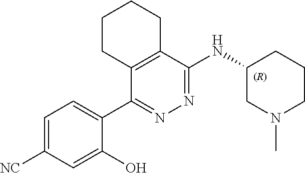

### Step 1: Synthesis of 4-(4-chloro-5,6,7,8-tetrahydrophthalazin-1-yl)-3-methoxybenzonitrile

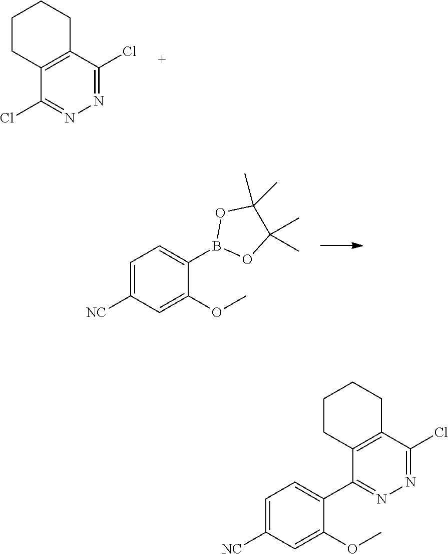 [SMILES: Clc1nnc(Cl)c2c1CCCC2 ; COc1cc(C#N)ccc1B1OC(C)(C)C(C)(C)O1 ; COc1cc(C#N)ccc1-c1nnc(Cl)c2c1CCCC2]

1,4-Dichloro-5,6,7,8-tetrahydrophthalazine (3.0 g, 14.77 mmol, 1.0 eq.), 3-methoxy-4-(4,4,5,5-tetramethyl-1,3,2-dioxaborolan-2-yl)benzonitrile (3.0 g, 11.81 mmol, 0.8 eq.), sodium bicarbonate (2.48 g, 29.54 mmol, 2.0 eq.) and Pd(PPh₃)₄ (853.3 mg, 0.73 mmol, 0.05 eq.) were added to a mixed solution of 1,4-dioxane (20.0 mL) and water (10.0 mL), and the reaction solution was reacted at 110° C. for 12 h in nitrogen atmosphere. After the reaction was completed as monitored by TLC, water (100.0 mL) was added, and ethyl acetate (100.0 mL) was added for extraction. The organic phase was dried over anhydrous sodium sulfate and filtered, the filtrate was concentrated under reduced pressure, and the crude product was purified by silica gel column chromatography (petroleum ether:ethyl acetate=15:1 to 3:1) to give the product (2.2 g, yield: 49.7%).

### Step 2: Synthesis of tert-butyl (R)-3-((4-(4-cyano-2-methoxyphenyl)-5,6,7,8-tetrahydrophthalazin-1-yl)amino)piperidine-1-carboxylate

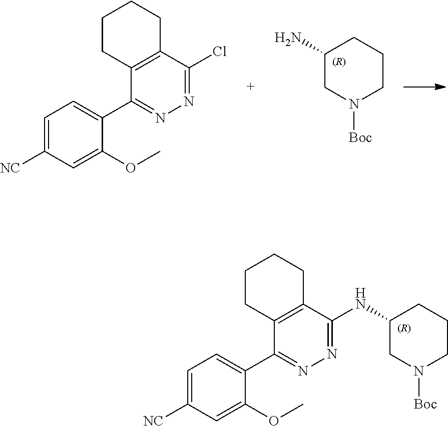

4-(4-Chloro-5,6,7,8-tetrahydrophthalazin-1-yl)-3-methoxybenzonitrile (1.5 g, 5.00 mmol, 1.0 eq.), tert-butyl (R)-3-aminopiperidine-1-carboxylate (2.0 g, 10.00 mmol, 2.0 eq.), cesium carbonate (4.88 g, 15.00 mmol, 3.0 q.), Pd₂(dba)₃ (457.8 mg, 0.50 mmol, 0.1 eq.) and 1,1′-binaphthyl-2,2′-bisdiphenylphosphine (622.67 mg, 1.00 mmol, 0.2 eq.) were added to 1,4-dioxane (30.0 mL), and the reaction solution was reacted at 90° C. for 12 h in nitrogen atmosphere. After the reaction was completed as monitored by TLC, water (100.0 mL) was added, and ethyl acetate (100.0 mL) was added for extraction. The organic phase was dried over anhydrous sodium sulfate and filtered, the filtrate was concentrated under reduced pressure, and the crude product was purified by silica gel column chromatography (specification of silica gel: 100-200 mesh, dichloromethane:methanol=100:1 to 80:1) to give the product (1.1 g, yield: 47.6%).

### Step 3: Synthesis of (R)-3-methoxy-4-(4-(piperidin-3-ylamino)-5,6,7,8-tetrahydrophthalazin-1-yl)benzonitrile

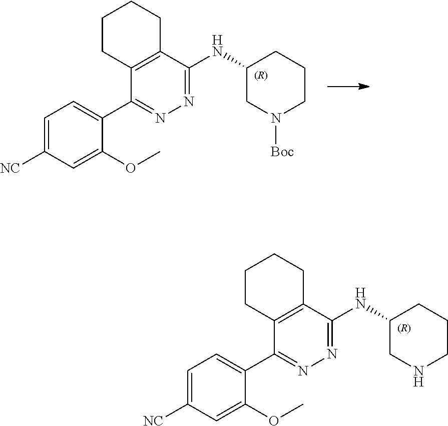

Tert-butyl (R)-3-((4-(4-cyano-2-methoxyphenyl)-5,6,7,8-tetrahydrophthalazin-1-yl)amino)piperidine-1-carboxylate (1.0 g, 2.15 mmol, 1.0 eq.) was added to dichloromethane (10.0 mL), then trifluoroacetic acid (5.0 mL) was added dropwise, and the reaction solution was reacted at room temperature for 2 h. After the reaction was completed as monitored by TLC, a saturated aqueous sodium bicarbonate solution was added to the system to adjust the pH to 7-8, followed by extraction with dichloromethane (100.0 mL). The organic phase was dried over anhydrous sodium sulfate and filtered, and the filtrate was concentrated under reduced pressure to give the product (750.0 mg, yield: 96.0%).

### Step 4: Synthesis of (R)-3-methoxy-4-(4-((1-methylpiperidin-3-yl)amino)-5,6,7,8-tetrahydrophthalazin-1-yl)benzonitrile

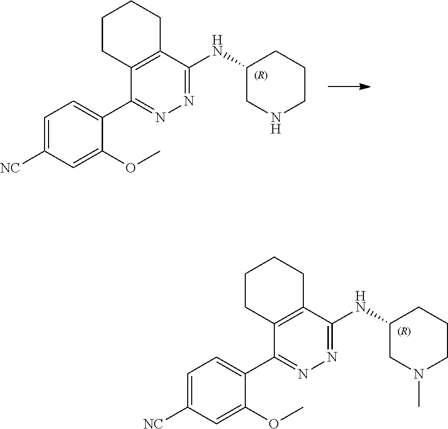

(R)-3-methoxy-4-(4-(piperidin-3-ylamino)-5,6,7,8-tetrahydrophthalazin-1-yl)benzonitrile (750.0 mg, 2.06 mmol, 1.0 eq.) and an aqueous formaldehyde solution (37%) (167.4 mg, 2.06 mmol, 1.0 eq.) were added to methanol (10.0 mL), the reaction solution was stirred at room temperature for 0.5 h, followed by addition of sodium cyanoborohydride (168.2 mg, 2.67 mmol, 1.3 eq.), and then the solution was reacted at room temperature for 2 h. After the reaction was completed as monitored by TLC, the system was concentrated under reduced pressure, and a saturated aqueous sodium bicarbonate solution (100.0 mL) was added, with stirring for 0.5 h, followed by extraction with dichloromethane (100.0 mL). The organic phase was dried over anhydrous sodium sulfate and filtered, the filtrate was concentrated under reduced pressure, and the crude product was purified by silica gel column chromatography (dichloromethane:methanol=40:1 to 30:1) to give the product (600.0 mg, yield: 77.2%).

### Step 5: Synthesis of (R)-3-hydroxy-4-(4-((1-methylpiperidin-3-yl)amino)-5,6,7,8-tetrahydrophthalazin-1-yl)benzonitrile

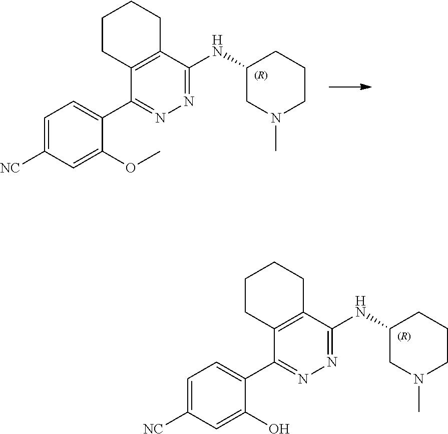

(R)-3-methoxy-4-(4-((1-methylpiperidin-3-yl)amino)-5,6,7,8-tetrahydrophthalazin-1-yl)benzonitrile (247.0 mg, 0.65 mmol, 1.0 eq.) was added to dichloromethane (10.0 mL), then boron tribromide (491.7 mg, 1.96 mmol, 3.0 eq.) was added dropwise under an ice bath, and the reaction solution was heated to room temperature for 4 h. After the reaction was completed as monitored by TLC, methanol (10.0 mL) was added to the system under an ice bath to quench the reaction, followed by concentration under reduced pressure, addition of dichloromethane (50.0 mL), and back extraction with water (50.0 mL). A saturated aqueous sodium bicarbonate solution was added to the aqueous phase to adjust the pH to 7-8, followed by extraction with dichloromethane (100.0 mL). The organic phase was dried over anhydrous sodium sulfate and filtered, the filtrate was concentrated under reduced pressure, and the crude product was purified by preparative thin-layer chromatography to give the product (130.0 mg, yield: 55.0%).

¹HNMR (300 MHz, DMSO-d6) δ (ppm): 10.59 (s, 1H), 7.33-7.30 (m, 3H), 5.94 (s, 1H), 4.46 (s, 1H), 2.99 (s, 1H), 2.55-2.51 (s, 3H), 2.42-2.29 (m, 6H), 1.88-1.69 (m, 5H), 1.61-1.60 (m, 4H).

Molecular formula: C₂₁H₂₅N₅O Precise molecular weight: 363.21 LC-MS (m/z)=364.14 [M+H]⁺.

### Example 64: Synthesis of 3-hydroxy-4-(4-(((1R,2R)-2-hydroxycyclohexyl)amino)-5,6,7,8-tetrahydrophthalazin-1-yl)benzonitrile (Compound 34a)

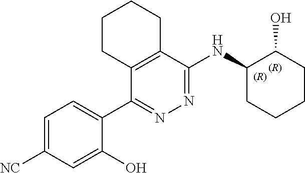

### Step 1: Synthesis of 3-(ethoxymethoxy)-4-(4-(((1R,2R)-2-hydroxycyclohexyl)amino)-5,6,7,8-tetrahydrophthalazin-1-yl)benzonitrile

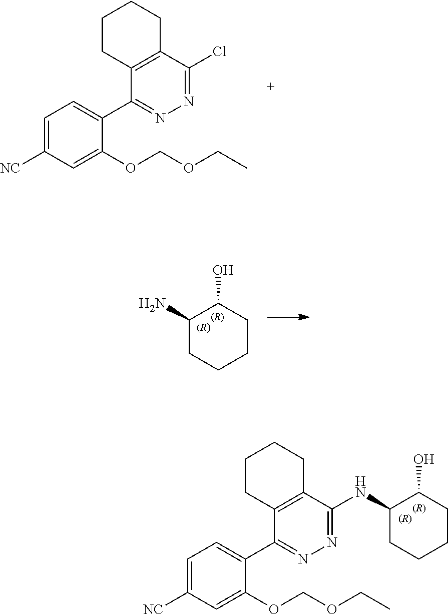

4-(4-Chloro-5,6,7,8-tetrahydrophthalazin-1-yl)-3-(ethoxymethoxy)benzonitrile (400.0 mg, 1.16 mmol, 1.0 eq.), (1R,2R)-2-aminocyclohexan-1-ol (267.9 mg, 2.32 mmol, 2.0 eq.), cesium carbonate (1.22 g, 3.48 mmol, 3.0 q.), Pd₂(dba)₃ (106.2 mg, 0.11 mmol, 0.1 eq.) and 1,1′-binaphthyl-2,2′-bisdiphenylphosphine (144.4 mg, 0.22 mmol, 0.2 eq.) were added to 1,4-dioxane (20.0 mL), and the reaction solution was reacted at 90° C. for 12 h in nitrogen atmosphere. After the reaction was completed as monitored by TLC, water (100.0 mL) was added, and ethyl acetate (100.0 mL) was added for extraction. The organic phase was dried over anhydrous sodium sulfate and filtered, the filtrate was concentrated under reduced pressure, and the crude product was purified by silica gel column chromatography (specification of silica gel: 100-200 mesh, dichloromethane:methanol=80:1 to 50:1) to give the product (251.0 mg, yield: 51.2%).

### Step 2: Synthesis of 3-hydroxy-4-(4-(((1R,2R)-2-hydroxycyclohexyl)amino)-5,6,7,8-tetrahydrophthalazin-1-yl)benzonitrile

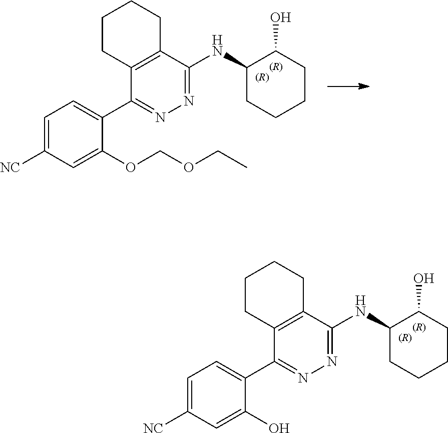

3-(Ethoxymethoxy)-4-(4-(((1R,2R)-2-hydroxycyclohexyl)amino)-5,6,7,8-tetrahydrophthalazin-1-yl)benzonitrile (251.0 mg, 0.59 mmol, 1.0 eq.) was added to ethanol (3.0 mL), a hydrogen chloride-ethanol solution (2.0 mol/L, 8.0 mL) was added dropwise, and the reaction solution was reacted at room temperature for 2 h. After the reaction was completed as monitored by TLC, a saturated aqueous sodium bicarbonate solution was added to the system to adjust the pH to 7-8, followed by extraction with dichloromethane (100.0 mL). The organic phase was dried over anhydrous sodium sulfate and filtered, the filtrate was concentrated under reduced pressure, and the crude product was purified by preparative thin-layer chromatography (dichloromethane:methanol=10:1) to give the product (77.0 mg, yield: 35.5%).

¹HNMR (400 MHz, DMSO-d6) δ (ppm): 10.55 (s, 1H), 7.33-7.32 (m, 2H), 7.25-7.24 (m, 1H), 5.62-5.60 (d, J=8 Hz, 1H), 4.81 (s, 1H), 3.93-3.87 (m, 1H), 3.52-3.47 (m, 1H), 2.42-2.29 (m, 5H), 2.11-2.08 (m, 1H), 1.99-1.92 (m, 1H), 1.77-1.59 (m, 8H).

Molecular formula: C₂₁H₂₄N₄O₂ Precise molecular weight: 364.19 LC-MS (m/z)=365.12 [M+H]⁺.

### Example 65: Synthesis of Compound (R)-5-fluoro-2-(4-((1-methylpiperidin-3-yl)amino)-5,6,7,8-tetrahydrophthalazin-1-yl)phenol (Compound 24a)

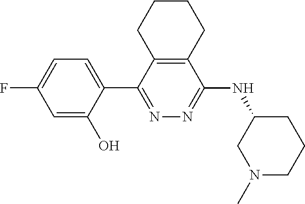 [SMILES: CN1CCC[C@@H](Nc2nnc(-c3ccc(F)cc3O)c3c2CCCC3)C1]

### Step 1: Synthesis of Intermediate 2,3,5,6,7,8-hexahydrophthalazine-1,4-dione

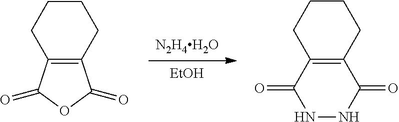 [SMILES: "EtOH" ; O=C1OC(=O)C2=C1CCCC2 ; "N2H4·H2O" ; O=c1[nH][nH]c(=O)c2c1CCCC2]

4,5,6,7-Tetrahydroisobenzofuran-1,3-dione (50 g, 329 mmol, 1.0 eq.) was dissolved in ethanol (500 mL), then hydrazine hydrate (20.64 g, 413 mmol, 1.25 eq.) was added, and the reaction solution was stirred at room temperature overnight. After the reaction was completed as detected by TLC, the reaction solution was filtered under vacuum, and the filter cake was dried to give the product (47 g, yield: 86.0%).

### Step 2: Synthesis of Intermediate 1,4-dichloro-5,6,7,8-tetrahydrophthalazine

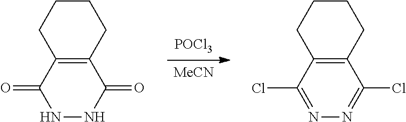 [SMILES: "MeCN" ; O=c1[nH][nH]c(=O)c2c1CCCC2 ; "POCl3" ; Clc1nnc(Cl)c2c1CCCC2]

2,3,5,6,7,8-Hexahydrophthalazine-1,4-dione (25 g, 150 mmol, 1.0 eq.) obtained in the previous step was dissolved in acetonitrile (250 mL), then phosphorus oxychloride (138 g, 904 mmol, 6.0 eq.) was added, and the reaction solution was stirred at 80° C. for 4 h. After the reaction was completed as detected by TLC, the reaction solution was slowly poured into ice water, and a large number of solids precipitated. After stirring for 1 h, the solution was filtered under vacuum, and the filter cake was dried to give the product (24 g, yield: 78.8%).

### Step 3: Synthesis of Intermediate tert-butyl (R)-3-((4-chloro-5,6,7,8-tetrahydrophthalazin-1-yl)amino)piperidine-1-carboxylate

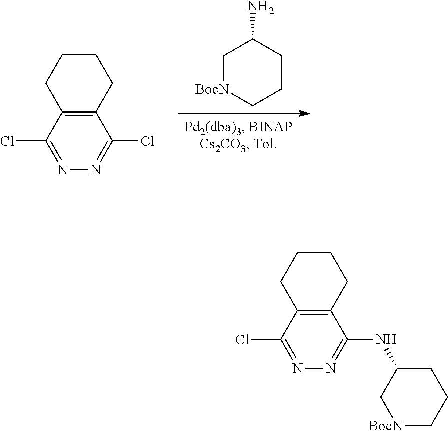

1,4-Dichloro-5,6,7,8-tetrahydrophthalazine (15 g, 73.9 mmol, 1.0 eq.) obtained in the previous step was dissolved in toluene (300 mL), then tert-butyl (R)-3-aminopiperidine-1-carboxylate (29.6 g, 147.8 mmol, 2.0 eq.), BINAP (9.2 g, 14.78 mmol, 0.2 eq.), cesium carbonate (72.3 g, 221.7 mmol, 3.0 eq.) and Pd₂(dba)₃ (6.8 g, 7.39 mmol, 0.1 eq.) were added successively, and the reaction solution was stirred at 90° C. overnight in nitrogen atmosphere. After the reaction was completed as detected by TLC, the reaction solution was filtered through celite under vacuum, the filtrate was concentrated, and the crude product was purified by silica gel column chromatography (dichloromethane:methanol=50:1 to 30:1) to give the product (13 g, yield: 43.9%).

### Step 4: Synthesis of Intermediate (R)-4-chloro-N-(piperidin-3-yl)-5,6,7,8-tetrahydrophthalazin-1-amine

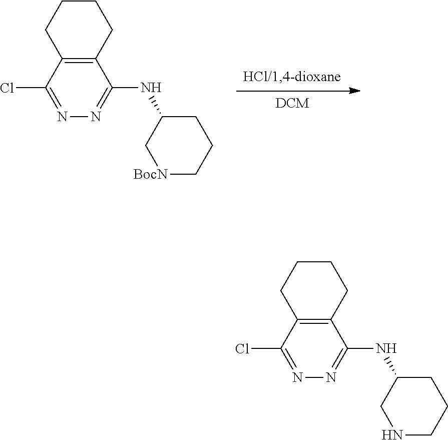

Tert-butyl (R)-3-((4-chloro-5,6,7,8-tetrahydrophthalazin-1-yl)amino)piperidine-1-carboxylate (13 g, 35.5 mmol, 1.0 eq.) obtained in the previous step was dissolved in dichloromethane (100 mL), then a 4 mol/L HCl/1,4-dioxane solution (45 mL, 5.0 eq.) was added, and the reaction solution was stirred at room temperature for 30 min. After the reaction was completed as detected by TLC, the reaction solution was poured into water, followed by liquid separation. Sodium bicarbonate solid was added to the aqueous phase to adjust the pH to 8, followed by concentration. The crude product was dissolved in DCM, filtered under vacuum, and concentrated to give the product (8 g, yield: 84.4%).

### Step 5: Synthesis of Intermediate (R)-4-chloro-N-(1-methylpiperidin-3-yl)-5,6,7,8-tetrahydrophthalazin-1-amine

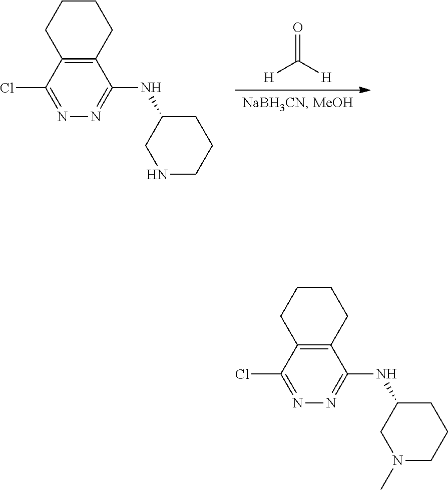

(R)-4-chloro-N-(piperidin-3-yl)-5,6,7,8-tetrahydrophthalazin-1-amine (8 g, 30 mmol, 1.0 eq.) obtained in the previous step was dissolved in methanol (100 mL), then an aqueous formaldehyde solution (37%) (2.43 g, 30 mmol, 1.0 eq.) was added, and the reaction solution was stirred at room temperature for 30 min. NaBH₃CN (2.45 g, 39 mmol, 1.3 eq.) was added, and the resulting solution was stirred at room temperature for 1 h. After the reaction was completed as detected by TLC, the reaction solution was concentrated, dissolved in DCM (100 mL), and washed with a saturated aqueous sodium bicarbonate solution (100 mL×2). The organic phase was dried and filtered under vacuum, the filtrate was concentrated, and the residue was purified by silica gel column chromatography (dichloromethane:methanol=50:1 to 30:1) to give the product (5 g, yield: 59.3%).

### Step 6: Synthesis of compound (R)-5-fluoro-2-(4-((1-methylpiperidin-3-yl)amino)-5,6,7,8-tetrahydrophthalazin-1-yl)phenol

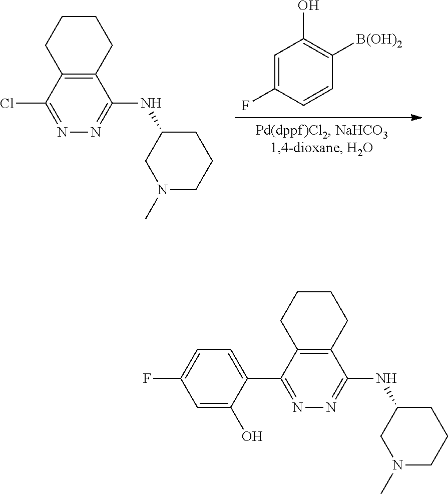

(R)-4-chloro-N-(1-methylpiperidin-3-yl)-5,6,7,8-tetrahydrophthalazin-1-amine (500 mg, 1.78 mmol, 1.0 eq.) obtained in the previous step was dissolved in 1,4-dioxane (4 mL) and water (2 mL), then (4-fluoro-2-hydroxyphenyl)boronic acid (333 mg, 2.14 mmol, 1.2 eq.), sodium bicarbonate (300 mg, 3.56 mmol, 2.0 eq.) and Pd(dppf)Cl₂ (130 mg, 0.178 mmol, 0.1 eq.) were added successively, and the reaction solution was stirred at 110° C. overnight in nitrogen atmosphere. After the reaction was completed as detected by TLC, the reaction solution was concentrated and extracted with dichloromethane (5 mL×3). The organic phase was dried and filtered under vacuum, the filtrate was concentrated, and the crude product was purified by silica gel column chromatography (dichloromethane:methanol=20:1 to 10:1) to give a crude product (600 mg), and the crude product (200 mg) was purified by preparative thin-layer chromatography to give the product (150 mg, yield: 71.0%).

¹H-NMR (400 MHz, DMSO-d6) δ (ppm): 10.26 (s, 1H), 7.13 (t, J=7.96 Hz, 1H), 6.74-6.69 (m, 2H), 5.69 (s, 1H), 4.34 (s, 1H), 3.35 (s, 1H), 3.08 (s, 1H), 2.79 (s, 1H), 2.38-2.31 (m, 8H), 1.87-1.52 (m, 8H).

Molecular formula: C₂₀H₂₅FN₄O Precise molecular weight: 356.20 LC-MS (m/z): 357.12 [M+H]⁺.

### Example 66: Synthesis of Compound (R)-2-fluoro-3-methyl-6-(4-((1-methylpiperidin-3-yl)amino)-5,6,7,8-tetrahydrophthalazin-1-yl)phenol (Compound 29a)

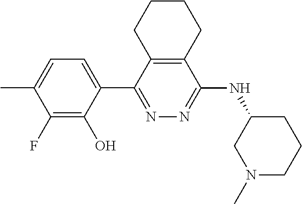 [SMILES: Cc1ccc(-c2nnc(N[C@@H]3CCCN(C)C3)c3c2CCCC3)c(O)c1F]

### Step 1: Synthesis of Intermediate 6-bromo-2-fluoro-3-methylphenol

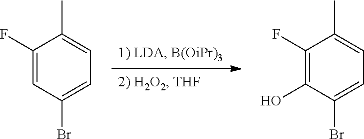

4-Bromo-2-fluoro-1-methylbenzene (2 g, 10.58 mmol, 1.0 eq.) was dissolved in dry tetrahydrofuran (15 mL), and the reaction solution was cooled to −70° C., followed by dropwise addition of a 2 mol/L solution of LDA in n-hexane (6 mL, 12 mmol, 1.13 eq.). After stirring for 2 h, triisopropyl borate (2.07 g, 11 mmol, 1.05 eq.) was added dropwise, with stirring for 20 min, then a 2 mol/L solution of LDA in n-hexane (2 mL, 4 mmol, 0.38 eq.) was added dropwise, with stirring for 20 min, and then triisopropyl borate (0.6 g, 3.2 mmol, 0.3 eq.) was added dropwise, with stirring for 1 h. The resulting solution was heated to 0° C., a solution of acetic acid (2 mL) in water (15 mL) was poured into the reaction solution, an aqueous hydrogen peroxide solution (30%, 5 mL) was added dropwise to the system, and the reaction was carried out overnight at room temperature. After the reaction was completed as detected by TLC, liquid separation was carried out, and the pH of an aqueous phase was adjusted to 6, followed by extraction with ethyl acetate (15 mL). The organic phases were combined, washed successively with a saturated aqueous sodium sulfite solution (15 mL) and a 1 mol/L aqueous hydrochloric acid solution (15 mL), and extracted with a 1 mol/L aqueous sodium hydroxide solution (15 mL×2). The pH of the aqueous phase was adjusted to 6, followed by extraction with dichloromethane (15 mL×2). The organic phases were combined, dried, and concentrated to give the product (2.1 g, yield: 96.8%).

### Step 2: Synthesis of Intermediate 1-bromo-2-(ethoxymethoxy)-3-fluoro-4-methylbenzene

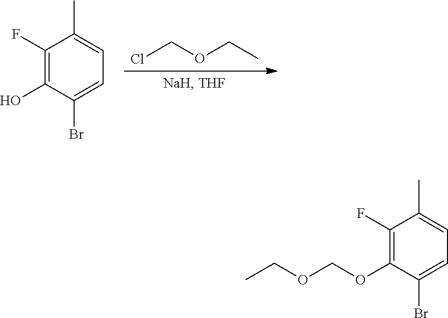

6-Bromo-2-fluoro-3-methylphenol (1 g, 4.88 mmol, 1.0 eq.) obtained in the previous step was dissolved in dry tetrahydrofuran (6 mL), 60% (mass fraction) NaH (293 mg, 7.32 mmol, 1.5 eq.) was added portionwise under an ice-water bath, with stirring for 30 min, followed by addition of chloromethyl ethyl ether (599 mg, 634 mmol, 1.3 eq.), and the reaction solution was stirred at room temperature overnight. After the reaction was completed as detected by TLC, water was added to the reaction solution to quench the reaction, followed by concentration, and then water (5 mL) and ethyl acetate (10 mL) were added, followed by liquid separation. The organic phase was dried and concentrated to give the product (1.1 g, yield: 85.7%).

### Step 3: Synthesis of Intermediate (2-(ethoxymethoxy)-3-fluoro-4-methylphenyl)boric acid

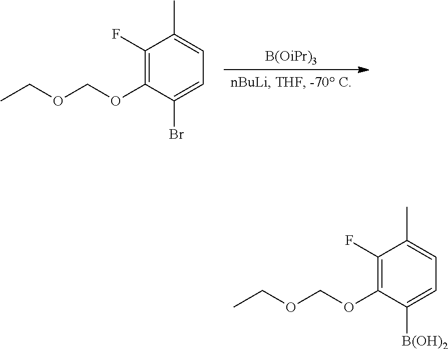 [SMILES: CCOCOc1c(Br)ccc(C)c1F ; "nBuLi, THF, -70° C." ; CCOCOc1c(B(O)O)ccc(C)c1F ; "B(OiPr)3"]

1-Bromo-2-(ethoxymethoxy)-3-fluoro-4-methylbenzene (526 mg, 2 mmol, 1.0 eq.) obtained in the previous step was dissolved in dry tetrahydrofuran (6 mL), and the reaction solution was cooled to −70° C., followed by dropwise addition of a 1.6 mol/L solution of n-butyllithium in n-hexane (1.9 mL, 3 mmol, 1.5 eq.). After stirring for 2 h, triisopropyl borate (489 mg, 2.6 mmol, 1.3 eq.) was added dropwise, and the resulting solution was stirred at room temperature for 3 h. After the reaction was completed as detected by TLC, water was added to the reaction solution to quench the reaction, followed by concentration. A 2 mol/L aqueous hydrochloric acid solution was added to adjust the pH to 6, followed by addition of water (5 mL) and ethyl acetate (10 mL), and liquid separation. The organic phase was dried and concentrated to give the product (450 mg, yield: 98.7%).

### Step 4: Synthesis of Intermediate (R)-4-(2-(ethoxymethoxy)-3-fluoro-4-methylphenyl)-N-(1-methylpiperidin-3-yl)-5,6,7,8-tetrahydrophthalazin-1-amine

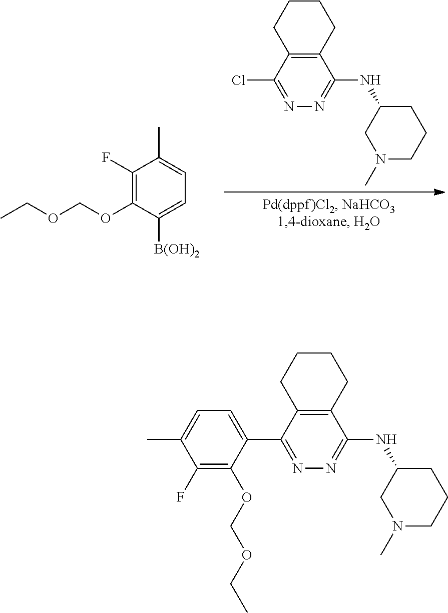 [SMILES: CCOCOc1c(B(O)O)ccc(C)c1F ; CN1CCC[C@@H](Nc2nnc(Cl)c3c2CCCC3)C1 ; "Pd(dppf)Cl2, NaHCO31,4-dioxane, H2O" ; CCOCOc1c(-c2nnc(N[C@@H]3CCCN(C)C3)c3c2CCCC3)ccc(C)c1F]

(R)-4-chloro-N-(1-methylpiperidin-3-yl)-5,6,7,8-tetrahydrophthalazin-1-amine (250 mg, 0.89 mmol, 1.0 eq.) was dissolved in 1,4-dioxane (2 mL) and water (1 mL), then (2-(ethoxymethoxy)-3-fluoro-4-methylphenyl)boronic acid (406 mg, 1.78 mmol, 2 eq.), sodium bicarbonate (150 mg, 1.78 mmol, 2.0 eq.) and Pd(dppf)Cl₂ (65 mg, 0.089 mmol, 0.1 eq.) were added successively, and the reaction solution was stirred at 110° C. overnight in nitrogen atmosphere. After the reaction was completed as detected by TLC, the reaction solution was concentrated, followed by addition of water (5 mL) and extraction with dichloromethane (5 mL×3). The organic phase was dried and filtered under vacuum, the filtrate was concentrated, and the crude product was purified by silica gel column chromatography (dichloromethane:methanol=20:1 to 10:1) to give the product (300 mg, yield: 78.7%).

### Step 5: Synthesis of compound (R)-2-fluoro-3-methyl-6-(4-((1-methylpiperidin-3-yl)amino)-5,6,7,8-tetrahydrophthalazin-1-yl)phenol

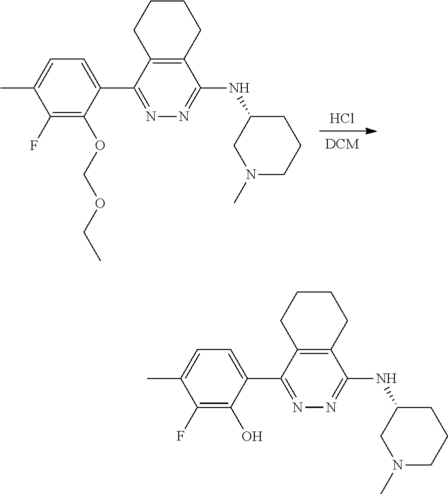

(R)-4-(2-(ethoxymethoxy)-3-fluoro-4-methylphenyl)-N-(1-methylpiperidin-3-yl)-5,6,7,8-tetrahydrophthalazin-1-amine (300 mg, 0.7 mmol, 1.0 eq.) obtained in the previous step was dissolved in dichloromethane (3 mL), then a 4 mol/L HCl/1,4-dioxane solution (0.88 mL, 5.0 eq.) was added, and the reaction solution was stirred at room temperature for 3 h. After the reaction was completed as detected by TLC, the reaction solution was poured into water, and the pH was adjusted to 8 with sodium bicarbonate solid, followed by liquid separation and extraction with dichloromethane (5 mL×2). The organic phase was dried, filtered under vacuum, and concentrated, and the crude product was purified by preparative thin-layer chromatography to give the product (200 mg, yield: 77.1%).

¹H-NMR (400 MHz, DMSO-d6) δ (ppm): 9.84 (s, 1H), 6.85 (d, J=7.84 Hz, 1H), 6.75 (t, J=7.4 Hz, 1H), 5.72 (s, 1H), 4.33 (s, 1H), 3.35 (s, 1H), 3.06 (s, 1H), 2.77 (s, 1H), 2.38-2.33 (m, 7H), 2.26 (s, 3H), 1.87-1.52 (m, 9H).

Molecular formula: C₂₁H₂₇FN₄O Precise molecular weight: 370.22 LC-MS (m/z): 371.20 [M+H]⁺.

### Example 67: Synthesis of (R)-4-(4-((1-cyclopropylpiperidin-3-yl)amino)-5,6,7,8-tetrahydrophthalazin-1-yl)-3-hydroxybenzonitrile (Compound 21a)

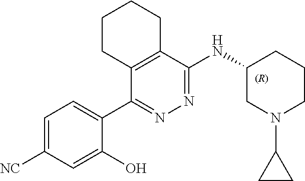

### Step 1: Synthesis of (R)-4-(4-((1-cyclopropylpiperidin-3-yl)amino)-5,6,7,8-tetrahydrophthalazin-1-yl)-3-(ethoxymethoxy)benzonitrile

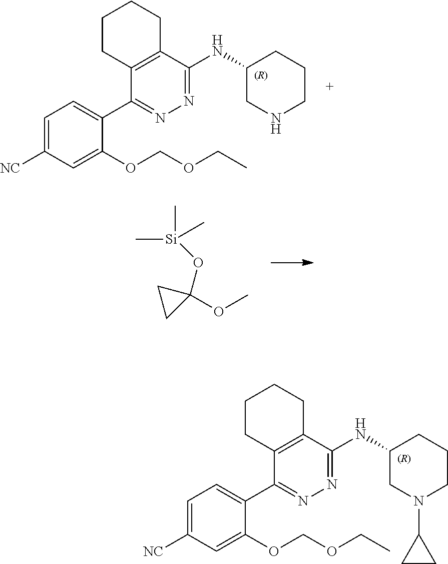

(R)-3-(ethoxymethoxy)-4-(4-(piperidin-3-ylamino)-5,6,7,8-tetrahydrophthalazin-1-yl)benzonitrile (370.0 mg, 0.90 mmol, 1.0 eq.), (1-methoxycyclopropoxy)trimethylsilane (627.5 mg, 3.60 mmol, 4.0 eq.), acetic acid (1.0 mL), cesium fluoride (205.0 mg, 1.35 mmol, 1.5 eq.) and sodium cyanoborohydride (282.7 mg, 4.50 mmol, 5.0 eq.) were added to methanol (15.0 mL), and the reaction solution was reacted at 60° C. for 12 h. After the reaction was completed as monitored by TLC, a saturated aqueous sodium bicarbonate solution (50.0 mL) was added, followed by extraction with dichloromethane (50.0 mL). The organic phase was dried over anhydrous sodium sulfate and filtered, the filtrate was concentrated under reduced pressure, and the crude product was purified by silica gel column chromatography (dichloromethane:methanol=40:1 to 30:1) to give the product (310.0 mg, yield: 77.1%).

### Step 2: Synthesis of (R)-4-(4-((1-cyclopropylpiperidin-3-yl)amino)-5,6,7,8-tetrahydrophthalazin-1-yl)-3-hydroxybenzonitrile

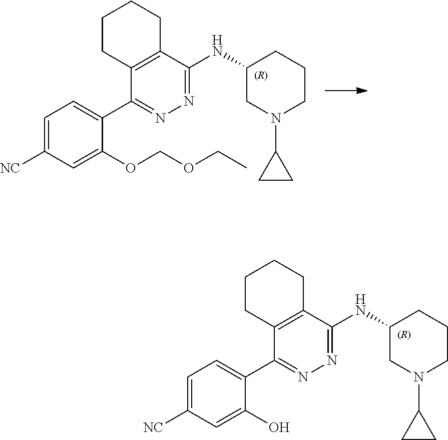

(R)-4-(4-((1-cyclopropylpiperidin-3-yl)amino)-5,6,7,8-tetrahydrophthalazin-1-yl)-3-(ethoxymethoxy)benzonitrile (280.0 mg, 0.62 mmol, 1.0 eq.) was added to ethanol (3.0 mL) and a hydrogen chloride-ethanol solution (2.0 mol/L) (5.0 mL), and the reaction solution was reacted at room temperature for 4 h. After the reaction was completed as monitored by TLC, the pH was adjusted to 7-8 with a saturated aqueous sodium bicarbonate solution, followed by extraction with dichloromethane (50.0 mL). The organic phase was dried over anhydrous sodium sulfate and filtered, the filtrate was concentrated under reduced pressure, and the crude product was purified by preparative thin-layer chromatography to give the product (130.0 mg, yield: 53.4%).

¹HNMR (400 MHz, DMSO-d6) δ (ppm): 10.50 (s, 1H), 7.33 (s, 2H), 7.24 (s, 1H), 5.61-5.59 (d, J=8 Hz, 1H), 4.19-4.17 (m, 1H), 3.10-3.08 (d, J=8 Hz, 1H), 2.80-2.78 (d, J=8 Hz, 1H), 2.39-2.16 (m, 5H), 1.84-1.45 (m, 9H), 0.87-0.84 (m, 1H), 0.41-0.37 (m, 2H), 0.34-0.30 (m, 2H).

Molecular formula: C₂₃H₂₇N₅O Precise molecular weight: 389.22 LC-MS (Pos, m/z)=390.15 [M+H]⁺.

### Example 68: Synthesis of (R)-5-methoxy-2-(4-((1-methylpiperidin-3-yl)amino)-5,6,7,8-tetrahydrophthalazin-1-yl)phenol (Compound 23a)

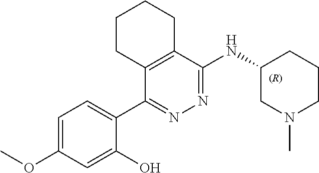

### Step 1: Synthesis of (R)-4-(2-(ethoxymethoxy)-4-methoxyphenyl)-N-(piperidin-3-yl)-5,6,7,8-tetrahydrophthalazin-1-amine

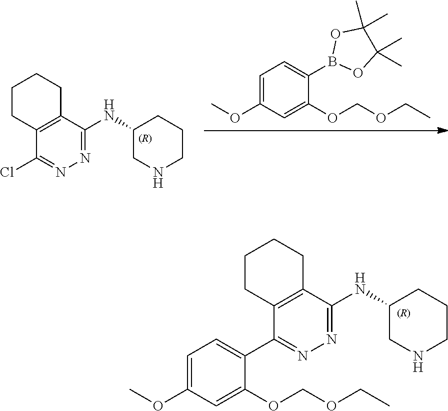

(R)-4-chloro-N-(piperidin-3-yl)-5,6,7,8-tetrahydrophthalazin-1-amine (504.0 mg, 1.88 mmol, 1.0 eq.), 2-(2-(ethoxymethoxy)-4-methoxyphenyl)-4,4,5,5-tetramethyl-1,3,2-dioxaborolane (1.74 g, 5.66 mmol, 3.0 eq.), sodium bicarbonate (631.7 mg, 7.52 mmol, 4.0 eq.) and Pd(dppf)Cl₂ (137.5 mg, 0.18 mmol, 0.1 eq.) were added to 1,4-dioxane (15.0 mL) and water (7.0 mL), and the reaction solution was reacted at 110° C. for 2 h in nitrogen atmosphere. After the reaction was completed as monitored by TLC, the system was cooled to room temperature, and water (50.0 mL) was added, followed by extraction with ethyl acetate (100.0 mL). The organic phase was dried over anhydrous sodium sulfate and filtered, the filtrate was concentrated under reduced pressure, and the crude product was purified by silica gel column chromatography (100-200 mesh silica gel, dichloromethane:methanol=40:1 to 10:1) to give the product (503.0 mg, yield: 64.8%).

### Step 2: Synthesis of (R)-4-(2-(ethoxymethoxy)-4-methoxyphenyl)-N-(1-methylpiperidin-3-yl)-5,6,7,8-tetrahydrophthalazin-1-amine

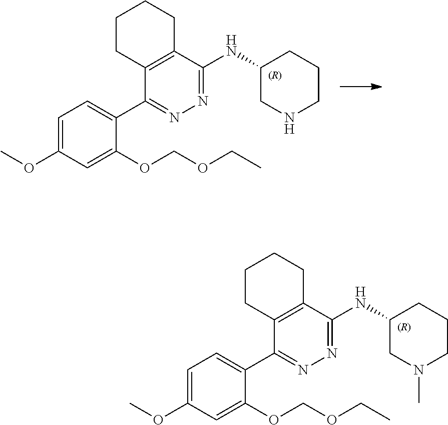

(R)-4-(2-(ethoxymethoxy)-4-methoxyphenyl)-N-(piperidin-3-yl)-5,6,7,8-tetrahydrophthalazin-1-amine (342.0 mg, 0.82 mmol, 1.0 eq.) was added to methanol (12.0 mL), then an aqueous formaldehyde solution (mass fraction: 37%, 80.7 mg, 0.99 mmol, 1.2 eq.) was added, and the reaction solution was reacted at room temperature for 0.5 h. Sodium cyanoborohydride (67.7 mg, 1.07 mmol, 1.3 eq.) was added, and the resulting solution was reacted for 1 h. After the reaction was completed as monitored by TLC, a saturated aqueous sodium bicarbonate solution was added to the system to adjust the pH to 7-8, followed by extraction with dichloromethane (50.0 mL). The organic phase was dried over anhydrous sodium sulfate and filtered, the filtrate was concentrated under reduced pressure, and the crude product was purified by silica gel column chromatography (100-200 mesh silica gel, dichloromethane:methanol=40:1 to 20:1) to give the product (270.0 mg, yield: 76.3%).

### Step 3: Synthesis of (R)-5-methoxy-2-(4-((1-methylpiperidin-3-yl)amino)-5,6,7,8-tetrahydrophthalazin-1-yl)phenol

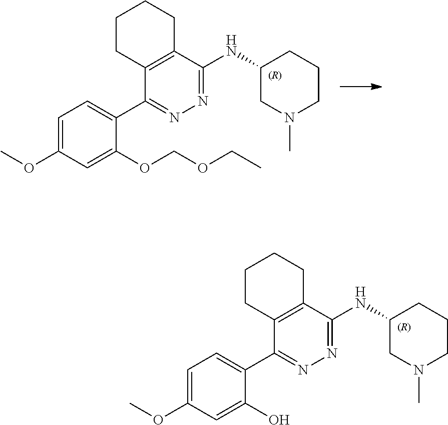

(R)-4-(2-(ethoxymethoxy)-4-methoxyphenyl)-N-(1-methylpiperidin-3-yl)-5,6,7,8-tetrahydrophthalazin-1-amine (270.0 mg, 0.63 mmol, 1.0 eq.) and a hydrogen chloride-ethanol solution (2.0 mol/L, 10.0 mL) were added to ethanol (10.0 mL), and the reaction solution was reacted at room temperature for 2 h. After the reaction was completed as monitored by TLC, a saturated aqueous sodium bicarbonate solution was added to the system to adjust the pH to 7-8, followed by extraction with dichloromethane (50.0 mL). The organic phase was dried over anhydrous sodium sulfate and filtered, the filtrate was concentrated under reduced pressure, and the crude product was purified by preparative thin-layer chromatography to give the product (130.0 mg, yield: 56.0%).

¹HNMR (400 MHz, DMSO-d6) δ (ppm): 9.94 (s, 1H), 7.05-7.03 (d, J=8 Hz, 1H), 6.46-6.44 (m, 2H), 5.59-5.58 (d, J=4 Hz, 1H), 4.28 (s, 1H), 3.74 (s, 3H), 3.00 (s, 1H), 2.70 (s, 1H), 2.37-2.34 (m, 4H), 2.28 (s, 3H), 2.09 (s, 1H), 1.85-1.82 (m, 1H), 1.75-1.73 (m, 3H), 1.59-1.47 (m, 5H).

Molecular formula: C₂₁H₂₈N₄O₂ Precise molecular weight: 368.22 LC-MS (Pos, m/z)=369.18 [M+H]⁺.

### Example 69: Synthesis of (R)-5-(difluoromethyl)-2-(4-((1-methylpiperidin-3-yl)amino)-5,6,7,8-tetrahydrophthalazin-1-yl)phenol (Compound 15a)

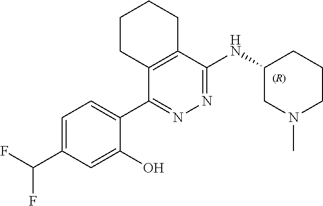

### Step 1: Synthesis of (R)-3-(ethoxymethoxy)-4-(4-((1-methylpiperidin-3-yl)amino)-5,6,7,8-tetrahydrophthalein-1-yl)benzaldehyde

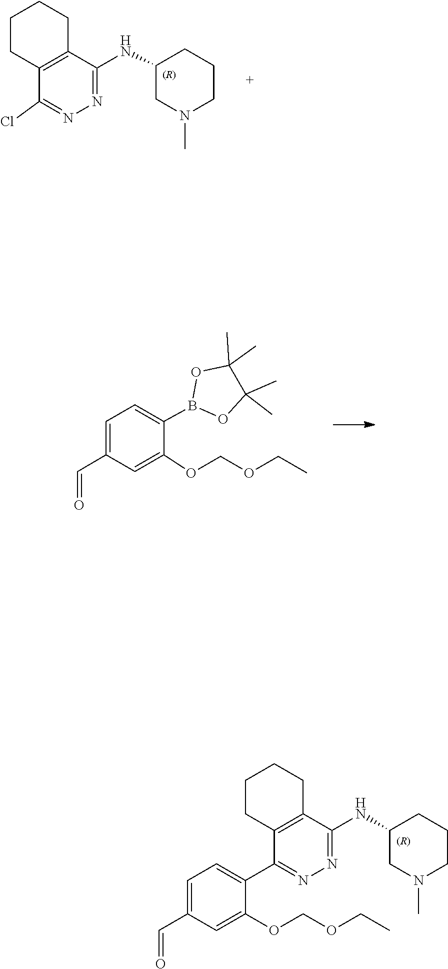

(R)-4-chloro-N-(1-methylpiperidin-3-yl)-5,6,7,8-tetrahydrophthalazin-1-amine (500.0 mg, 1.78 mmol, 1.0 eq.), 3-(ethoxymethoxy)-4-(4,4,5,5-tetramethyl-1,3,2-dioxaborolan-2-yl)benzaldehyde (817.7 mg, 2.67 mmol, 1.5 eq.), sodium bicarbonate (299.0 mg, 3.56 mmol, 2.0 eq.) and Pd(dppf)Cl₂ (65.1 mg, 0.08 mmol, 0.05 eq.) were added to 1,4-dioxane (10.0 mL) and water (5.0 mL), and the reaction solution was reacted at 110° C. for 4 h in nitrogen atmosphere. After the reaction was completed as monitored by TLC, water (100.0 mL) was added, and ethyl acetate (200.0 mL) was added for extraction. The organic phase was dried over anhydrous sodium sulfate and filtered, the filtrate was concentrated under reduced pressure, and the crude product was purified by silica gel column chromatography (100-200 mesh silica gel, dichloromethane:methanol=40:1 to 10:1) to give the product (430.0 mg, yield: 56.9%).

### Step 2: Synthesis of (R)-4-(4-(difluoromethyl)-2-(ethoxymethoxy)phenyl)-N-(1-methylpiperidin-3-yl)-5,6,7,8-tetrahydrophthalazin-1-amine

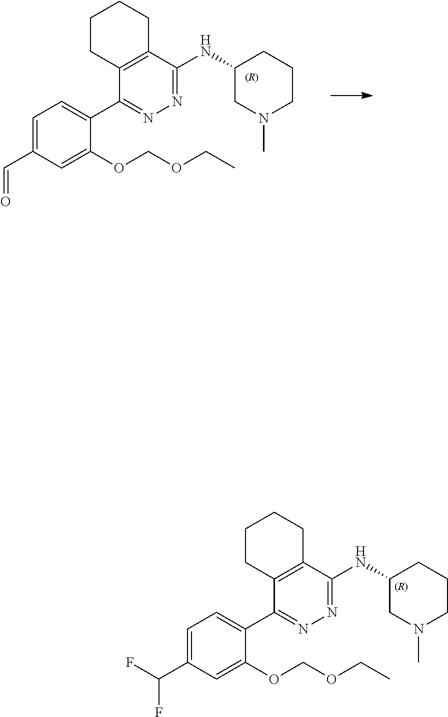

(R)-3-(ethoxymethoxy)-4-(4-((1-methylpiperidin-3-yl)amino)-5,6,7,8-tetrahydrophthalein-1-yl)benzaldehyde (430.0 mg, 1.01 mmol, 1.0 eq.) was added to dichloromethane (10.0 mL), then diethylaminosulfur trifluoride (244.8 mg, 1.51 mmol, 1.5 eq.) was added dropwise under an ice bath, and the reaction solution was warmed to room temperature and stirred for 12 h. After the reaction was completed as monitored by TLC, a saturated aqueous sodium carbonate solution was added to adjust the pH to 8-9 under an ice bath, followed by extraction with dichloromethane (100.0 mL). The organic phase was dried over anhydrous sodium sulfate and filtered, the filtrate was concentrated under reduced pressure, and the crude product was purified by preparative thin-layer chromatography to give the product (220.0 mg, yield: 48.7%).

### Step 3: Synthesis of (R)-5-(difluoromethyl)-2-(4-((1-methylpiperidin-3-yl)amino)-5,6,7,8-tetrahydrophthalazin-1-yl)phenol

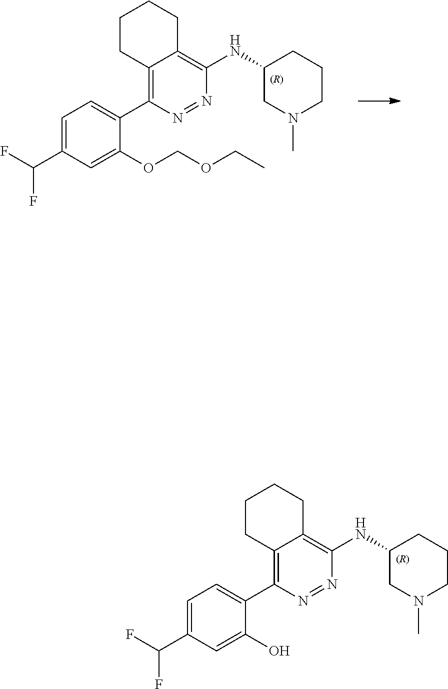

(R)-4-(4-(difluoromethyl)-2-(ethoxymethoxy)phenyl)-N-(1-methylpiperidin-3-yl)-5,6,7,8-tetrahydrophthalazin-1-amine (180.0 mg, 0.40 mmol, 1.0 eq.) and a hydrogen chloride-1,4-dioxane solution (4.0 mol/L, 3.0 mL) were added to dichloromethane (1.0 mL), and the reaction solution was stirred at room temperature for 2 h. After the reaction was completed as monitored by TLC, a saturated aqueous sodium bicarbonate solution was added to the system to adjust the pH to 7-8, followed by extraction with dichloromethane (100.0 mL). The organic phase was dried over anhydrous sodium sulfate and filtered, the filtrate was concentrated under reduced pressure, and the crude product was purified by preparative thin-layer chromatography to give the product (30.0 mg, yield: 19.2%).

¹HNMR (400 MHz, DMSO-d6) δ (ppm): 10.16 (s, 1H), 7.25-7.23 (d, J=8 Hz, 1H), 7.14 (s, 0.20H), 7.06-7.03 (m, 2H), 7.00 (s, 0.43H), 6.86 (s, 0.24H), 5.54-5.52 (d, J=8 Hz, 1H), 4.28-4.22 (m, 1H), 2.90-2.88 (d, J=8 Hz, 1H), 2.59-2.57 (d, J=8 Hz, 1H), 2.38-2.31 (m, 4H), 2.18 (s, 3H), 1.99-1.91 (m, 2H), 1.84-1.40 (m, 8H).

Molecular formula: C₂₁H₂₆F₂N₄O Precise molecular weight: 388.21 LC-MS (Pos, m/z)=389.14 [M+H]⁺.

### Example 70: Synthesis of 3-hydroxy-4-(4-(((R)-1-methylpiperidin-3-yl)amino)-5,8-dihydro-5,8-ethanophthalazin-1-yl)benzonitrile (Compound 9a)

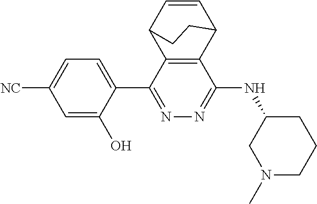 [SMILES: CN1CCC[C@@H](Nc2nnc(-c3ccc(C#N)cc3O)c3c2C2C=CC3CC2)C1]

### Step 1: Synthesis of dimethyl bicyclo[2.2.2]octa-2,5-diene-2,3-dicarboxylate

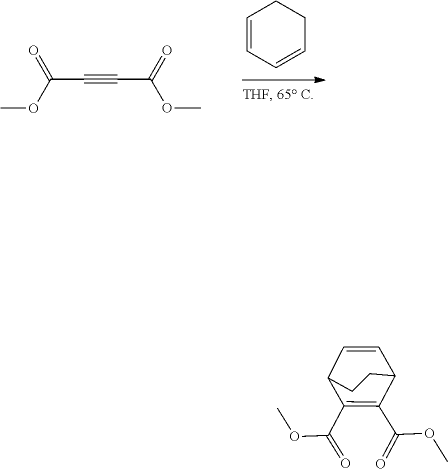

Dimethyl acetylenedicarboxylate (10.0 g, 70.4 mmol, 1.0 eq.) and 1,3-cyclohexadiene (6.77 g, 84.5 mmol, 1.2 eq.) were dissolved in THF (50 mL), and the reaction solution was heated to 65° C. in nitrogen atmosphere and reacted for 20 h, then cooled to room temperature, and concentrated under reduced pressure. The crude product was separated by silica gel column chromatography (PE:EA=50:1) to give the product (12.5 g, yield: 79.9%).

### Step 2: Synthesis of 5,8-dihydro-5,8-ethanophthalazine-1,4-diol

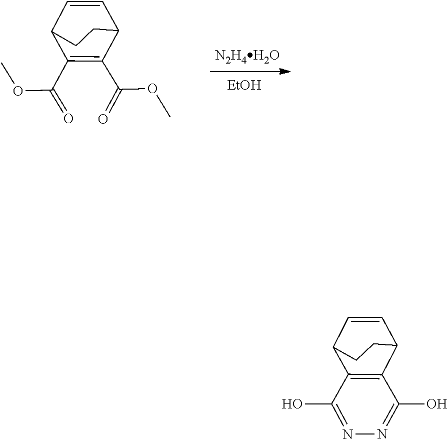

Dimethyl bicyclo[2.2.2]octa-2,5-diene-2,3-dicarboxylate (12.0 g, 54.0 mmol, 1.0 eq.) was dissolved in EtOH (50 mL), followed by addition of hydrazine hydrate (85%, 32.0 g, 540 mmol, 10.0 eq.), and the reaction solution was reacted at room temperature for 30 h, and concentrated to give the product (10.0 g, yield: 97.4%).

### Step 3: Synthesis of 1,4-dichloro-5,8-dihydro-5,8-ethanophthalazine

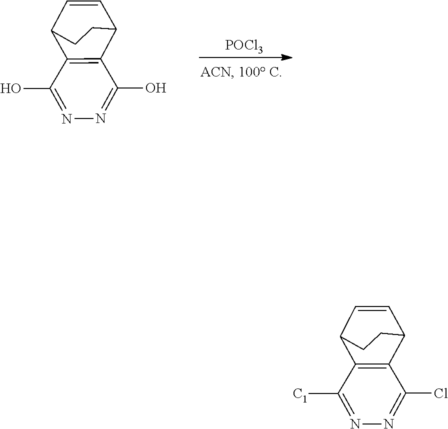

5,8-Dihydro-5,8-ethanophthalazine-1,4-diol (10.0 g, 52.6 mmol, 1.0 eq.) was added to ACN (50 mL), then POCl₃ (50 mL) was added, and the reaction solution was heated to 100° C. under an oil bath and reacted for 5 h, then cooled to room temperature, concentrated, washed with water (50 mL), and filtered under vacuum to give the product (2.60 g, yield: 21.8%).

### Step 4: Synthesis of tert-butyl (3R)-3-((4-chloro-5,8-dihydro-5,8-ethanophthalazin-1-yl)amino)piperidine-1-carboxylate

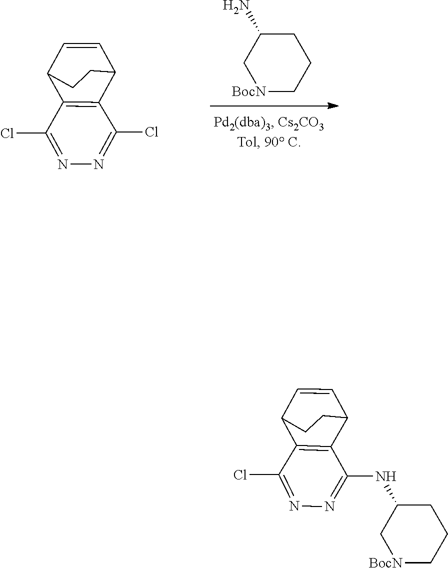 [SMILES: Clc1nnc(Cl)c2c1C1C=CC2CC1 ; CC(C)(C)OC(=O)N1CCC[C@@H](N)C1 ; CC(C)(C)OC(=O)N1CCC[C@@H](Nc2nnc(Cl)c3c2C2C=CC3CC2)C1]

1,4-Dichloro-5,8-dihydro-5,8-ethanophthalazine (2.60 g, 11.4 mmol, 1.0 eq.), tert-butyl (R)-3-aminopiperidine-1-carboxylate (2.52 g, 12.6 mmol, 1.1 eq.), Pd₂(dba)₃ (1.05 g, 1.14 mmol, 0.1 eq.), BINAP (1.42 g, 2.28 mmol, 0.2 eq.) and Cs₂CO₃ (7.46 g, 22.4 mmol, 2.0 eq.) were added successively to toluene (50 mL), and the reaction solution was heated to 90° C. in nitrogen atmosphere and reacted for 2 h, and then cooled to room temperature. Water was added to quench the reaction, followed by extraction with EA (30 mL×2). The organic phase was dried over anhydrous sodium sulfate and filtered, the filtrate was concentrated under reduced pressure, and the crude product was separated by silica gel column chromatography (DCM:MeOH=100:1 to 50:1) to give the product (2.70 g, yield: 60.3%).

### Step 5: Synthesis of tert-butyl (3R)-3-((4-(4-cyano-2-(ethoxymethoxy)phenyl)-5,8-dihydro-5,8-ethanophthalazin-1-yl)amino)piperidine-1-carboxylate

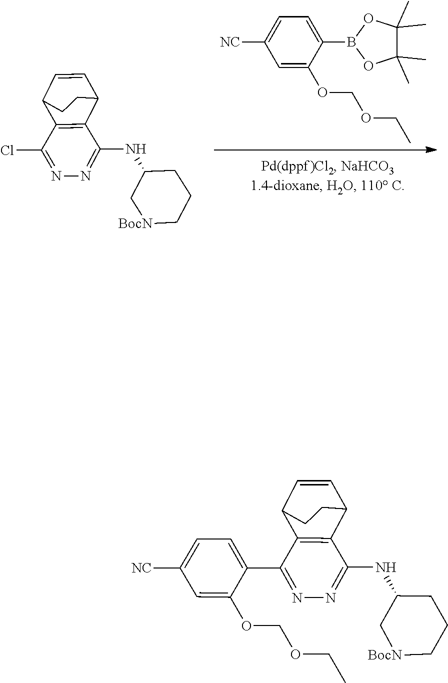 [SMILES: CC(C)(C)OC(=O)N1CCC[C@@H](Nc2nnc(Cl)c3c2C2C=CC3CC2)C1 ; CCOCOc1cc(C#N)ccc1B1OC(C)(C)C(C)(C)O1 ; CCOCOc1cc(C#N)ccc1-c1nnc(N[C@@H]2CCCN(C(=O)OC(C)(C)C)C2)c2c1C1C=CC2CC1]

3-(Ethoxymethoxy)-4-(4,4,5,5-tetramethyl-1,3,2-dioxaborolan-2-yl)benzonitrile (349 mg, 1.15 mmol, 1.5 eq.), (R)-4-chloro-N-(1-(tetrahydro-2H-pyran-4-yl)piperidin-3-yl)phthalazin-1-amine (300 mg, 0.767 mmol, 1.0 eq.), Pd(dppf)Cl₂ (28.1 mg, 0.0384 mmol, 0.05 eq.) and NaHCO₃ (128 mg, 1.53 mmol, 2.0 eq.) were added successively to 1,4-dioxane (6 mL) and water (3 mL), and the reaction solution was heated to 110° C. in nitrogen atmosphere and reacted for 2 h, and then cooled to room temperature. Water was added to quench the reaction, followed by extraction with EA (20 mL×2). The organic phase was dried over anhydrous sodium sulfate and filtered, the filtrate was concentrated under reduced pressure, and the crude product was separated by silica gel column chromatography (DCM:MeOH=50:1) to give the product (280 mg, yield: 68.6%).

### Step 6: Synthesis of 3-hydroxy-4-(4-(((R)-piperidin-3-yl)amino)-5,8-dihydro-5,8-ethanophthalazin-1-yl)benzonitrile

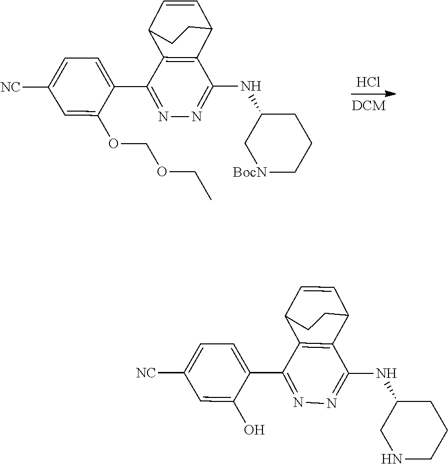 [SMILES: CCOCOc1cc(C#N)ccc1-c1nnc(N[C@@H]2CCCN(C(=O)OC(C)(C)C)C2)c2c1C1C=CC2CC1 ; Cl ; N#Cc1ccc(-c2nnc(N[C@@H]3CCCNC3)c3c2C2C=CC3CC2)c(O)c1]

Tert-butyl (3R)-3-((4-(4-cyano-2-(ethoxymethoxy)phenyl)-5,8-dihydro-5,8-ethanophthalazin-1-yl)amino)piperidine-1-carboxylate (270 mg, 0.508 mmol, 1.0 eq.) was dissolved in DCM (3 mL), then a solution of hydrogen chloride in 1,4-dioxane (4 mol/L, 1.27 mL, 5.08 mmol, 10.0 eq.) was added dropwise, and the reaction solution was stirred at room temperature for 2 h. Water (20 mL) was added to quench the reaction, followed by washing with DCM (15 mL×2), and a saturated aqueous NaHCO₃ solution was added to the aqueous phase to adjust the pH to 8, followed by extraction with DCM (20 mL×3). The organic phase was dried over anhydrous sodium sulfate and filtered, and the filtrate was concentrated under reduced pressure to give the product (168 mg, yield: 88.6%).

### Step 7: Synthesis of 3-hydroxy-4-(4-(((R)-1-methylpiperidin-3-yl)amino)-5,8-dihydro-5,8-ethanophthalazin-1-yl)benzonitrile

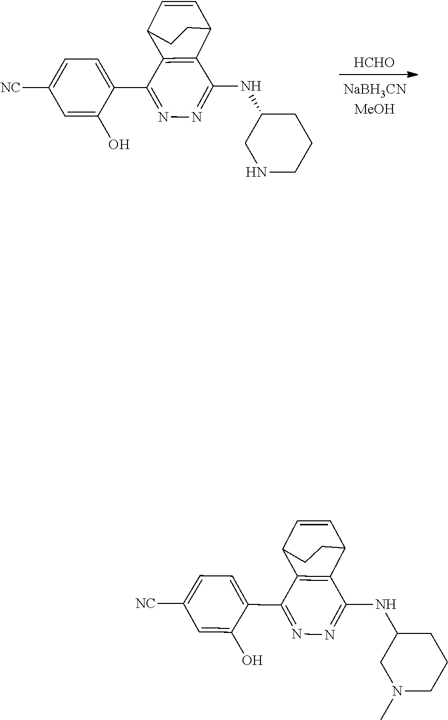 [SMILES: N#Cc1ccc(-c2nnc(N[C@@H]3CCCNC3)c3c2C2C=CC3CC2)c(O)c1 ; "HCHO" ; CN1CCCC(Nc2nnc(-c3ccc(C#N)cc3O)c3c2C2C=CC3CC2)C1]

3-Hydroxy-4-(4-(((R)-piperidin-3-yl)amino)-5,8-dihydro-5,8-ethanophthalazin-1-yl)benzonitrile (168 mg, 0.434 mmol, 1.0 eq.) was dissolved in MeOH (4 mL), then an aqueous formaldehyde solution (37%, 42.3 mg, 0.521 mmol, 1.2 eq.) was added, and the reaction solution was stirred at room temperature for 20 min. NaBH₃CN (32.7 mg, 0.521 mmol, 1.2 eq.) was then added, and the resulting solution was reacted for 1 h, concentrated, and dissolved in DCM. A saturated aqueous NaHCO₃ solution was added to adjust the pH to 8, followed by extraction with DCM (20 mL×2). The organic phase was dried over anhydrous sodium sulfate and filtered, the filtrate was concentrated under reduced pressure, and the crude product was separated by preparative thin-layer chromatography (DCM:MeOH=10:1) to give the product (36.8 mg, yield: 21.1%).

¹HNMR (400 MHz, DMSO-d6) δ (ppm): 10.75 (s, 1H), 7.44 (d, J=8.0 Hz, 1H), 7.39-7.36 (m, 1H), 7.33 (s, 1H), 6.49-6.41 (m, 3H), 4.48 (s, 1H), 4.29 (s, 1H), 3.76 (d, J=5.6 Hz, 1H), 3.15 (s, 2H), 2.83 (s, 1H), 2.34 (s, 3H), 2.08 (s, 1H), 1.92 (s, 1H), 1.78 (s, 1H), 1.64-1.61 (m, 1H), 1.47-1.39 (m, 4H), 1.34-1.31 (m, 1H).

Molecular formula: C₂₃H₂₅N₅O Precise molecular weight: 387.21 LC-MS (Pos, m/z)=388.13 [M+H]⁺.

### Example 71: Synthesis of (R)-5-cyclopropyl-2-(4-((1-methylpiperidin-3-yl)amino)-5,6,7,8-tetrahydro-5,8-ethanophthalazin-1-yl)phenol (Compound 10a)

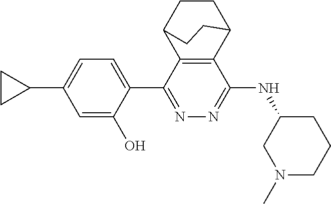 [SMILES: CN1CCC[C@@H](Nc2nnc(-c3ccc(C4CC4)cc3O)c3c2C2CCC3CC2)C1]

### Step 1: Synthesis of (R)-4-(4-cyclopropyl-2-methoxyphenyl)-N-(1-methylpiperidin-3-yl)-5,6,7,8-tetrahydro-5,8-ethanophthalazin-1-amine

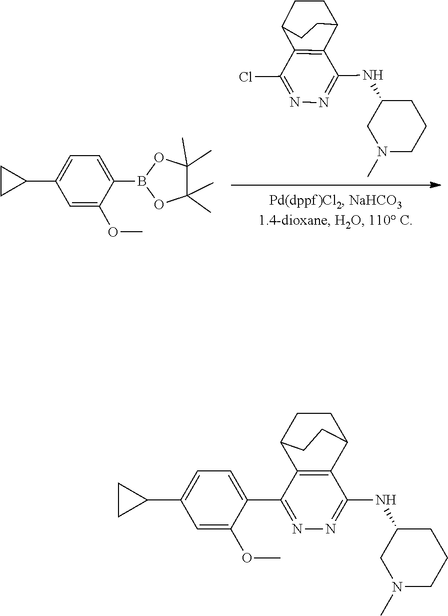 [SMILES: COc1cc(C2CC2)ccc1B1OC(C)(C)C(C)(C)O1 ; CN1CCC[C@@H](Nc2nnc(Cl)c3c2C2CCC3CC2)C1 ; COc1cc(C2CC2)ccc1-c1nnc(N[C@@H]2CCCN(C)C2)c2c1C1CCC2CC1]

2-(4-Cyclopropyl-2-methoxyphenyl)-4,4,5,5-tetramethyl-1,3,2-dioxaborolane (313 mg, 1.14 mmol, 1.1 eq.), (R)-4-chloro-N-(1-methylpiperidin-3-yl)-5,6,7,8-tetrahydro-5,8-ethanophthalazin-1-amine (320 mg, 1.04 mmol, 1.0 eq.), Pd(dppf)Cl₂ (76.1 mg, 0.104 mmol, 0.1 eq.) and NaHCO₃ (175 mg, 2.08 mmol, 2.0 eq.) were added successively to 1,4-dioxane (6 mL) and H₂O (3 mL), and the reaction solution was heated to 110° C. in nitrogen atmosphere and reacted for 2 h, and then cooled to room temperature. Water was added to quench the reaction, followed by extraction with EA (20 mL×3). The organic phase was dried over anhydrous sodium sulfate and filtered, the filtrate was concentrated under reduced pressure, and the crude product was separated by silica gel column chromatography (DCM:MeOH=50:1 to 10:1) to give the product (360 mg, yield: 82.5%).

### Step 2: Synthesis of (R)-5-cyclopropyl-2-(4-((1-methylpiperidin-3-yl)amino)-5,6,7,8-tetrahydro-5,8-ethanophthalazin-1-yl)phenol

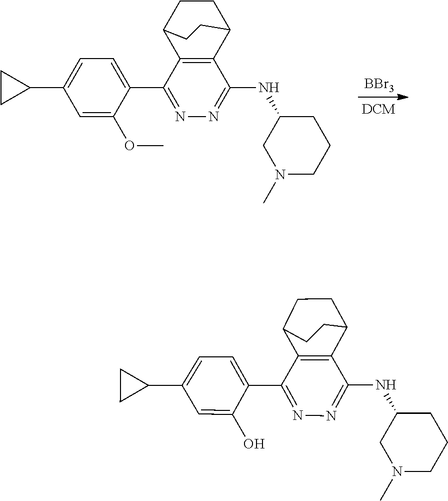 [SMILES: COc1cc(C2CC2)ccc1-c1nnc(N[C@@H]2CCCN(C)C2)c2c1C1CCC2CC1 ; "BBr3" ; CN1CCC[C@@H](Nc2nnc(-c3ccc(C4CC4)cc3O)c3c2C2CCC3CC2)C1]

((R)-4-(4-cyclopropyl-2-methoxyphenyl)-N-(1-methylpiperidin-3-yl)-5,6,7,8-tetrahydro-5,8-ethanophthalazin-1-amine (340 mg, 0.812 mmol, 1.0 eq.) was dissolved in DCM (10 mL), and the reaction solution was cooled to −10° C. BBr₃ (611 mg, 2.44 mmol, 3.0 eq.) was added dropwise, and the reaction was continued for 2 h. MeOH was added to quench the reaction, and a saturated aqueous NaHCO₃ solution was added to adjust the pH to 8, followed by extraction with DCM (20 mL×2). The organic phase was dried over anhydrous sodium sulfate and filtered, and the filtrate was concentrated under reduced pressure. The crude product was separated by preparative thin-layer chromatography (DCM:MeOH=8:1) to give the product (38.0 mg, yield: 11.6%).

¹HNMR (400 MHz, CDCl₃) δ(ppm): 11.75 (s, 1H), 7.19 (d, J=8.0 Hz, 1H), 6.79 (d, J=1.6 Hz, 1H), 6.69-6.67 (m, 1H), 5.24 (s, 1H), 4.48 (s, 1H), 3.59 (s, 1H), 5.10 (s, 1H), 2.66 (s, 1H), 2.50 (s, 1H), 2.29 (s, 3H), 2.14 (m, 1H), 1.93-1.82 (m, 6H), 1.76-1.70 (m, 2H), 1.46-1.39 (m, 4H), 1.02-1.97 (m, 2H), 0.91-0.88 (m, 1H), 0.79-0.85 (m, 2H).

Molecular formula: C₂₅H₃₂N₄O Precise molecular weight: 404.26 LC-MS (Pos, m/z)=405.17 [M+H]⁺.

### Example 72: Synthesis of 2-(4-(((cis)-3-hydroxy-3-methylcyclobutyl)amino)-5,6,7,8-tetrahydrophthalazin-1-yl)-5-(trifluoromethyl)phenol (Compound 32a)

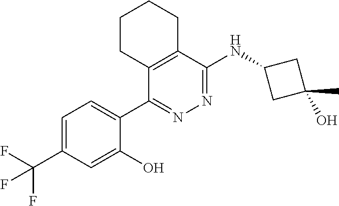 [SMILES: C[C@]1(O)C[C@@H](Nc2nnc(-c3ccc(C(F)(F)F)cc3O)c3c2CCCC3)C1]

### Step 1: Synthesis of 1-chloro-4-(2-(ethoxymethoxy)-4-(trifluoromethyl)phenyl)-5,6,7,8-tetrahydrophthalazine

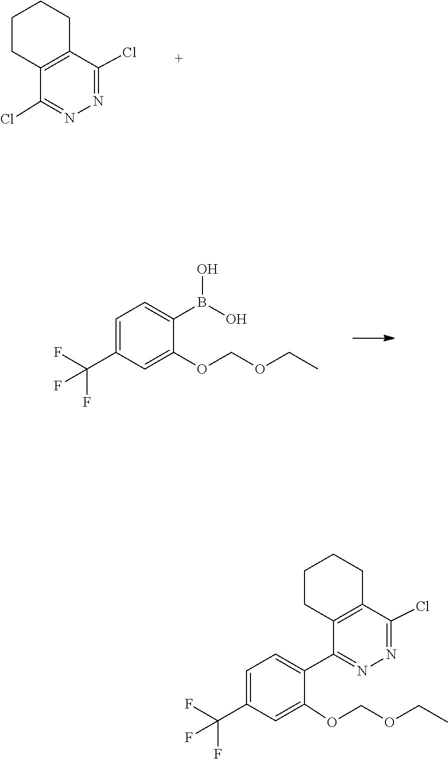 [SMILES: Clc1nnc(Cl)c2c1CCCC2 ; CCOCOc1cc(C(F)(F)F)ccc1B(O)O ; CCOCOc1cc(C(F)(F)F)ccc1-c1nnc(Cl)c2c1CCCC2]

1,4-Dichloro-5,6,7,8-tetrahydrophthalazine (518.0 mg, 2.55 mmol, 1.0 eq.), (2-(ethoxymethoxy)-4-(trifluoromethyl)phenyl) boronic acid (740.7 mg, 2.80 mmol, 1.1 eq.), sodium bicarbonate (428.0 mg, 5.10 mmol, 2.0 eq.) and Pd(dppf)Cl₂ (93.2 mg, 0.12 mmol, 0.05 eq.) were added to a mixed solution of 1,4-dioxane (10.0 mL) and water (4.0 mL), and the reaction solution was reacted at 110° C. for 3 h in nitrogen atmosphere. After the reaction was completed as monitored by TLC, water (100.0 mL) was added, and ethyl acetate (100.0 mL) was added for extraction. The organic phase was dried over anhydrous sodium sulfate and filtered, the filtrate was concentrated under reduced pressure, and the crude product was purified by silica gel column chromatography (petroleum ether:ethyl acetate=20:1 to 8:1) to give the product (450.0 mg, yield: 45.6%).

### Step 2: Synthesis of (cis)-3-((4-(2-(ethoxymethoxy)-4-(trifluoromethyl)phenyl)-5,6,7,8-tetrahydrophthalazin-1-yl)amino)-1-methylcyclobutan-1-ol

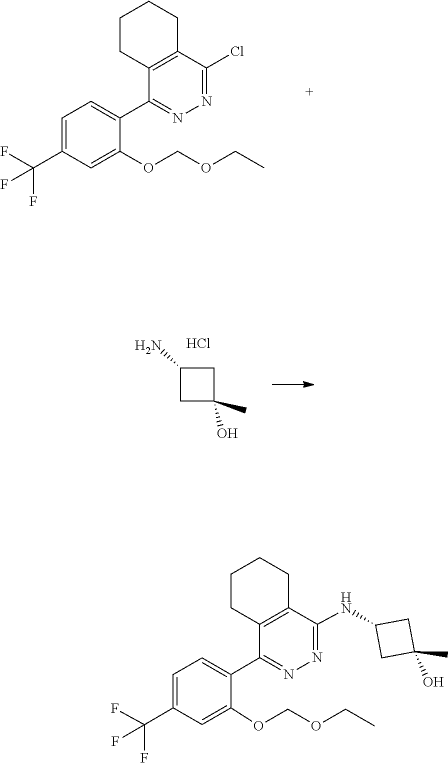

1-Chloro-4-(2-(ethoxymethoxy)-4-(trifluoromethyl)phenyl)-5,6,7,8-tetrahydrophthalazine (400.0 mg, 1.03 mmol, 1.0 eq.), (cis)-3-amino-1-methylcyclobutan-1-ol hydrochloride (213.4 mg, 1.55 mmol, 1.5 eq.), cesium carbonate (673.7 mg, 2.06 mmol, 2.0 ethoxyq.), Pd₂(dba)₃ (94.6 mg, 0.10 mmol, 0.1 eq.) and 1,1′-binaphthyl-2,2′-bisdiphenylphosphine (128.7 mg, 0.20 mmol, 0.2 eq.) were added to 1,4-dioxane (15.0 mL), and the reaction solution was reacted at 90° C. for 12 h in nitrogen atmosphere. After the reaction was completed as monitored by TLC, water (100.0 mL) was added, and ethyl acetate (100.0 mL) was added for extraction. The organic phase was dried over anhydrous sodium sulfate and filtered, the filtrate was concentrated under reduced pressure, and the crude product was purified by silica gel column chromatography (specification of silica gel: 100-200 mesh, dichloromethane:methanol=80:1 to 50:1) to give the product (380.0 mg, yield: 81.5%).

### Step 3: Synthesis of 2-(4-(((cis)-3-hydroxy-3-methylcyclobutyl)amino)-5,6,7,8-tetrahydrophthalazin-1-yl)-5-(trifluoromethyl)phenol

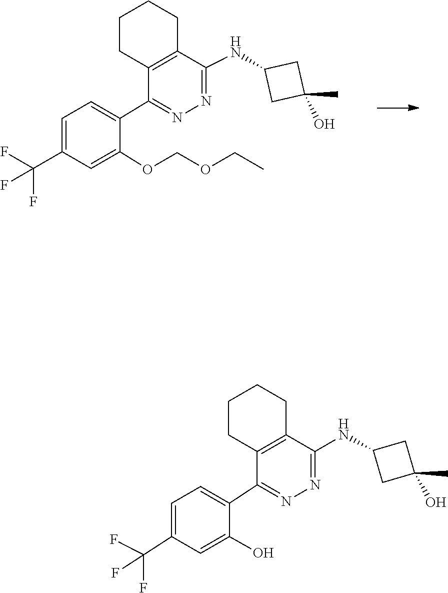 [SMILES: CCOCOc1cc(C(F)(F)F)ccc1-c1nnc(N[C@H]2C[C@@](C)(O)C2)c2c1CCCC2 ; C[C@]1(O)C[C@@H](Nc2nnc(-c3ccc(C(F)(F)F)cc3O)c3c2CCCC3)C1]

(Cis)-3-((4-(2-(ethoxymethoxy)-4-(trifluoromethyl)phenyl)-5,6,7,8-tetrahydrophthalazin-1-yl)amino)-1-methylcyclobutan-1-ol (380.0 mg, 0.84 mmol, 1.0 eq.) was added to dichloromethane (2.0 mL), then a hydrogen chloride-1,4-dioxane solution (4.0 mol/L, 6.0 mL) was added dropwise, and the reaction solution was reacted at room temperature for 2 h. After the reaction was completed as monitored by TLC, a saturated aqueous sodium bicarbonate solution was added to the system to adjust the pH to 7-8, followed by extraction with dichloromethane (100.0 mL). The organic phase was dried over anhydrous sodium sulfate and filtered, and the filtrate was concentrated under reduced pressure to give the product (222.0 mg, yield: 67.2%).

¹HNMR (400 MHz, DMSO-d6) δ (ppm): 11.14 (s, 1H), 8.28 (s, 1H), 7.48-7.46 (d, J=8 Hz, 1H), 7.42 (s, 1H), 7.30-7.28 (d, J=8 Hz, 1H), 5.16 (s, 1H), 4.05-4.00 (m, 1H), 3.57 (s, 1H), 2.61-2.59 (m, 2H), 2.42-2.41 (m, 2H), 2.30-2.25 (m, 2H), 1.79-1.65 (m, 4H), 1.30 (s, 3H).

Molecular formula: C₂₀H₂₂F₃N₃O₂ Precise molecular weight: 393.17 LC-MS (m/z)=394.11 [M+H]⁺.

### Example 73: Synthesis of 3-hydroxy-4-(4-(((cis)-3-hydroxy-3-methylcyclobutyl)amino)-5,6,7,8-tetrahydrophthalazin-1-yl)benzonitrile (Compound 33a)

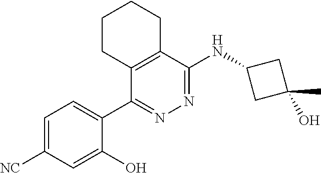 [SMILES: C[C@]1(O)C[C@@H](Nc2nnc(-c3ccc(C#N)cc3O)c3c2CCCC3)C1]

### Step 1: Synthesis of 3-(ethoxymethoxy)-4-(4-(((cis)-3-hydroxy-3-methylcyclobutyl)amino)-5,6,7,8-tetrahydrophthalazin-1-yl)benzonitrile

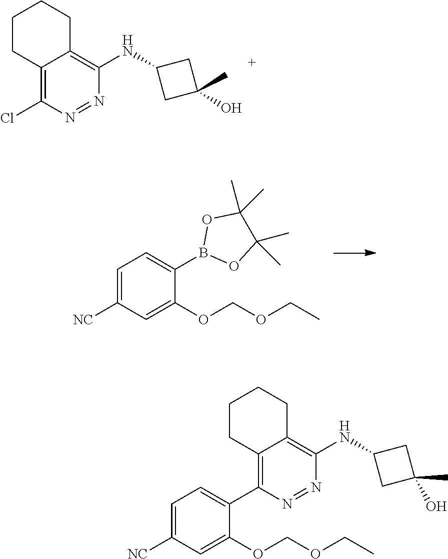 [SMILES: C[C@]1(O)C[C@@H](Nc2nnc(Cl)c3c2CCCC3)C1 ; CCOCOc1cc(C#N)ccc1B1OC(C)(C)C(C)(C)O1 ; CCOCOc1cc(C#N)ccc1-c1nnc(N[C@H]2C[C@@](C)(O)C2)c2c1CCCC2]

(Cis)-3-((4-chloro-5,6,7,8-tetrahydrophthalazin-1-yl)amino)-1-methylcyclobutan-1-ol (180.0 mg, 0.67 mmol, 1.0 eq.), 3-(ethoxymethoxy)-4-(4,4,5,5-tetramethyl-1,3,2-dioxaborolan-2-yl)benzonitrile (305.5 mg, 1.00 mmol, 1.5 eq.), sodium bicarbonate (112.9 mg, 1.34 mmol, 2.0 eq.) and Pd(dppf)Cl₂ (24.5 mg, 0.03 mmol, 0.05 eq.) were added to a mixed solution of 1,4-dioxane (10.0 mL) and water (5.0 mL), and the reaction solution was reacted at 110° C. for 4 h in nitrogen atmosphere. After the reaction was completed as monitored by TLC, water (100.0 mL) was added, and ethyl acetate (100.0 mL) was added for extraction. The organic phase was dried over anhydrous sodium sulfate and filtered, the filtrate was concentrated under reduced pressure, and the crude product was purified by silica gel column chromatography (dichloromethane:methanol=80:1 to 30:1) to give the product (130.0 mg, yield: 47.3%).

### Step 2: Synthesis of 3-hydroxy-4-(4-(((cis)-3-hydroxy-3-methylcyclobutyl)amino)-5,6,7,8-tetrahydrophthalazin-1-yl)benzonitrile

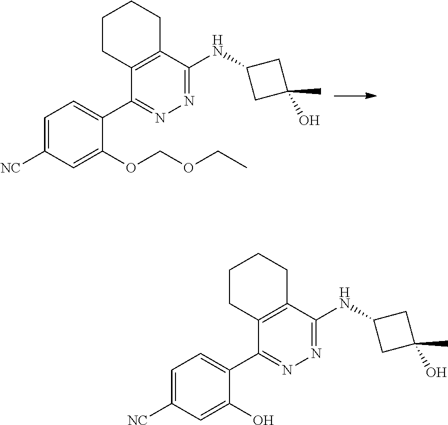 [SMILES: CCOCOc1cc(C#N)ccc1-c1nnc(N[C@H]2C[C@@](C)(O)C2)c2c1CCCC2 ; C[C@]1(O)C[C@@H](Nc2nnc(-c3ccc(C#N)cc3O)c3c2CCCC3)C1]

3-(Ethoxymethoxy)-4-(4-(((cis)-3-hydroxy-3-methylcyclobutyl)amino)-5,6,7,8-tetrahydrophthalazin-1-yl)benzonitrile (130.0 mg, 0.31 mmol, 1.0 eq.) was added to dichloromethane (2.0 mL), then a hydrogen chloride-1,4-dioxane solution (4.0 mol/L, 4.0 mL) was added dropwise, and the reaction solution was reacted at room temperature for 2 h. After the reaction was completed as monitored by TLC, a saturated aqueous sodium bicarbonate solution was added to the system to adjust the pH to 7-8, followed by extraction with dichloromethane (50.0 mL). The organic phase was dried over anhydrous sodium sulfate and filtered, and the filtrate was concentrated under reduced pressure to give the product (100.0 mg, yield: 90.0%).

¹HNMR (400 MHz, DMSO-d6) δ (ppm): 10.53 (s, 1H), 7.32 (s, 2H), 7.24 (s, 1H), 6.17-6.16 (t, J=4 Hz, 1H), 4.93 (s, 1H), 4.11-4.06 (m, 1H), 2.39-2.29 (m, 6H), 2.09-2.05 (m, 2H), 1.75-1.59 (m, 4H), 1.29 (s, 3H).

Molecular formula: C₂₀H₂₂N₄O₂ Precise molecular weight: 350.17 LC-MS (m/z)=351.08 [M+H]⁺.

### Example 74: Synthesis of Compound (R)-5-methyl-2-(4-((1-methylpiperidin-3-yl)amino)-6,7,8,9-tetrahydro-5H-cyclohepta[d]pyridazin-1-yl)phenol (Compound 26a)

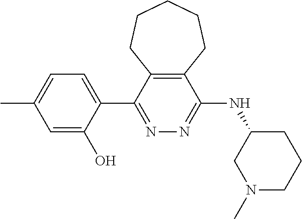 [SMILES: Cc1ccc(-c2nnc(N[C@@H]3CCCN(C)C3)c3c2CCCCC3)c(O)c1]

### Step 1: Synthesis of Intermediate methyl 2-(((trifluoromethyl)sulfonyl)oxy)cyclohept-1-ene-1-carboxylate

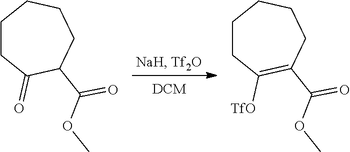 [SMILES: COC(=O)C1CCCCCC1=O ; COC(=O)C1=C(OS(=O)(=O)C(F)(F)F)CCCCC1]

NaH (6.64 g, 166 mmol, 1.2 eq.) was suspended in dichloromethane (600 mL), then a solution of methyl 2-oxocycloheptane-1-carboxylate (23.5 g, 138 mmol, 1.0 eq.) in dichloromethane (50 mL) was added dropwise at 0° C., and the reaction solution was stirred at 0° C. for 30 min. A solution of trifluoromethanesulfonic anhydride (47 g, 166 mmol, 1.2 eq.) in dichloromethane (100 mL) was added dropwise, the resulting solution was reacted at 25° C. for 30 min, followed by supplementation with dichloromethane (450 mL), and then the reaction was carried out at 25° C. for 16 h. Water was added dropwise to the reaction solution to quench the reaction, followed by liquid separation. The organic phase was concentrated, and the crude product was purified by silica gel column chromatography (petroleum ether:ethyl acetate=30:1) to give a crude product (35 g, yield: 83%).

### Step 2: Synthesis of Intermediate 5,6,7,8-tetrahydro-1H-cyclohepta[c]furan-1,3(4H)-dione

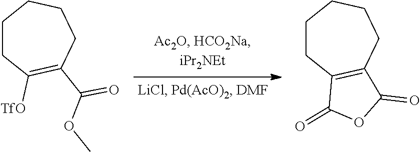 [SMILES: COC(=O)C1=C(OS(=O)(=O)C(F)(F)F)CCCCC1 ; O=C1OC(=O)C2=C1CCCCC2]

Sodium formate (4.1 g, 59.60 mmol, 3.0 eq.), DIEA (5.12 g, 39.74 mmol, 2.0 eq.) and acetic anhydride (4 g, 39.74 mmol, 2.0 eq.) were dissolved in DMF (20 mL) with stirring for 30 min, then DMF (40 mL), methyl 2-(((trifluoromethyl)sulfonyl)oxy)cyclohept-1-ene-1-carboxylate (6 g, 19.87 mmol, 1.0 eq.), palladium acetate (220 mg, 0.99 mmol, 0.05 eq.) and lithium chloride (2.5 g, 59.60 mmol, 3.0 eq.) were added, and the reaction solution was reacted at 25° C. for 12 h. After the materials reacted completely as monitored by TLC, the reaction solution was poured into a solution of ethyl acetate and 2 mol/L hydrochloric acid, and after stirring for 5 min, liquid separation was carried out. The organic phase was dried and concentrated, and the crude product was purified by silica gel column chromatography (petroleum ether:ethyl acetate=5:1) to give a crude product (3 g, yield: 91%).

### Step 3: Synthesis of Intermediate 2,3,6,7,8,9-hexahydro-1H-cyclohepta[d]pyridazine-1,4(5H)-dione

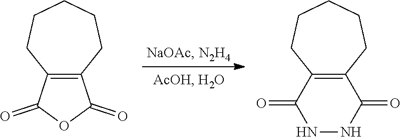 [SMILES: O=C1OC(=O)C2=C1CCCCC2 ; O=c1[nH][nH]c(=O)c2c1CCCCC2]

5,6,7,8-Tetrahydro-1H-cyclohepta[c]furan-1,3(4H)-dione (3 g, 18.07 mmol, 1.0 eq.) was added to acetic acid (18 mL) and water (20 mL), then sodium acetate (2.2 g, 27.1 mmol, 1.5 eq.) and hydrazine hydrate (1.35 g, 27.1 mmol, 1.5 eq.) were added, and the reaction solution was reacted at 110° C. for 18 h. The solvent was removed under reduced pressure, the crude product was slurried with MTBE and filtered under vacuum, and the filter cake was washed with ethyl acetate to give a crude product (3.5 g).

### Step 4: Synthesis of Intermediate 1,4-dichloro-6,7,8,9-tetrahydro-5H-cyclohepta[d]pyridazine

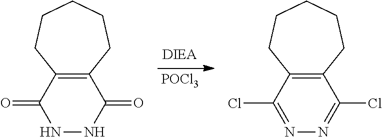

2,3,6,7,8,9-Hexahydro-1H-cyclohepta[d]pyridazine-1,4(5H)-dione (3.5 g, 19.4 mmol, 1.0 eq.) and DIPEA (2.5 g, 19.4 mmol, 1.0 eq.) were added to phosphorus oxychloride (20 mL), and the reaction solution was reacted at 110° C. for 40 min. The solvent was removed under reduced pressure, the crude product was diluted with dichloromethane, and a saturated aqueous sodium bicarbonate solution was added to adjust the pH to alkalinity, followed by liquid separation. The organic phase was dried and concentrated, and the crude product was purified by silica gel column chromatography (petroleum ether:ethyl acetate=5:1) to give the product (800 mg, two-step yield: 20%).

### Step 5: Synthesis of Intermediate tert-butyl (R)-3-((4-chloro-6,7,8,9-tetrahydro-5H-cyclohepta[d]pyridazin-1-yl)amino)piperidine-1-carboxylate

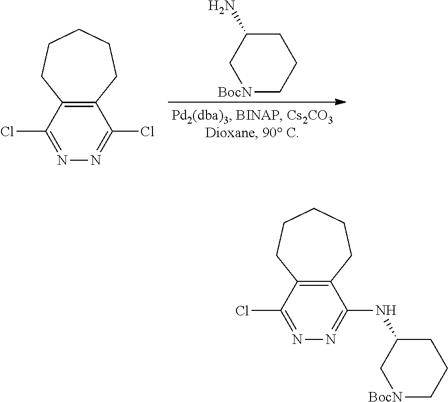 [SMILES: Clc1nnc(Cl)c2c1CCCCC2 ; CC(C)(C)OC(=O)N1CCC[C@@H](N)C1 ; CC(C)(C)OC(=O)N1CCC[C@@H](Nc2nnc(Cl)c3c2CCCCC3)C1]

1,4-Dichloro-6,7,8,9-tetrahydro-5H-cyclohepta[d]pyridazine (800 mg, 3.69 mmol, 1.0 eq.) was added to dioxane (15 mL), then Pd₂(dba)₃ (337 mg, 0.37 mmol, 0.1 eq.), BINAP (458 mg, 0.74 mmol, 0.2 eq.), cesium carbonate (3.6 g, 11.06 mmol, 3.0 eq.) and tert-butyl (R)-3-aminopiperidine-1-carboxylate (737 mg, 3.69 mmol, 1.0 eq.) were added, and the reaction solution was reacted at 110° C. for 3 h in nitrogen atmosphere. After there remained a small number of materials as detected by TLC, the reaction solution was filtered through celite under vacuum, the filtrate was concentrated, and the crude product was purified by silica gel column chromatography (petroleum ether:ethyl acetate=5:1 to 1:1) to give the product (1 g, yield: 71%).

### Step 6: Synthesis of Intermediate tert-butyl (R)-3-((4-(2-methoxy-4-methylphenyl)-6,7,8,9-tetrahydro-5H-cyclohepta[d]pyridazin-1-yl)amino)piperidine-1-carboxylate

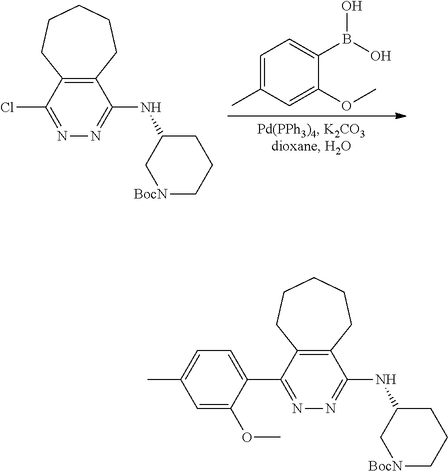

Tert-butyl (R)-3-((4-chloro-6,7,8,9-tetrahydro-5H-cyclohepta[d]pyridazin-1-yl)amino)piperidine-1-carboxylate (600 mg, 1.58 mmol, 1.0 eq.) was added to dioxane (10 mL) and water (2 mL), then Pd(PPh₃)₄ (182 mg, 0.16 mmol, 0.1 eq.), potassium carbonate (653 mg, 4.74 mmol, 2.5 eq.) and (2-methoxy-4-methylphenyl)boronic acid (288 mg, 1.74 mmol, 1.1 eq.) were added, and the reaction solution was reacted at 90° C. for 5 h in nitrogen atmosphere. After the materials reacted completely as monitored by TLC, the reaction solution was filtered through celite under vacuum, the filtrate was concentrated, and the crude product was purified by silica gel column chromatography (petroleum ether:ethyl acetate=3:1 to 1:1) to give the product (660 mg, yield: 90%).

### Step 7: Synthesis of Intermediate (R)-5-methyl-2-(4-(piperidin-3-ylamino)-6,7,8,9-tetrahydro-5H-cyclohepta[d]pyridazin-1-yl)phenol

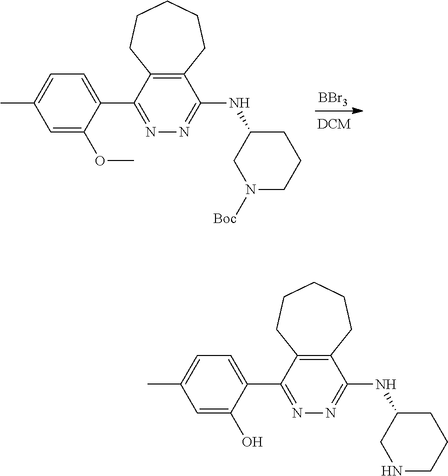 [SMILES: COc1cc(C)ccc1-c1nnc(N[C@@H]2CCCN(C(=O)OC(C)(C)C)C2)c2c1CCCCC2 ; "BBr3" ; Cc1ccc(-c2nnc(N[C@@H]3CCCNC3)c3c2CCCCC3)c(O)c1]

Tert-butyl (R)-3-((4-(2-methoxy-4-methylphenyl)-6,7,8,9-tetrahydro-5H-cyclohepta[d]pyridazin-1-yl)amino)piperidine-1-carboxylate (660 mg, 1.43 mmol, 1.0 eq.) was dissolved in dichloromethane (12 mL), then boron tribromide (1.8 g, 7.08 mmol, 5.0 eq.) was added, and the reaction solution was reacted at 25° C. for 1 h. After the materials reacted completely as monitored by TLC, water was added to the reaction solution at 0° C. to quench the reaction, followed by liquid separation. A saturated aqueous sodium bicarbonate solution was added to the aqueous phase to adjust the pH to alkalinity, followed by extraction with dichloromethane. The organic phase was dried and concentrated to give the product (380 mg, yield: 76%).

### Step 8: Synthesis of (R)-5-methyl-2-(4-((1-methylpiperidin-3-yl)amino)-6,7,8,9-tetrahydro-5H-cyclohepta[d]pyridazin-1-yl)phenol

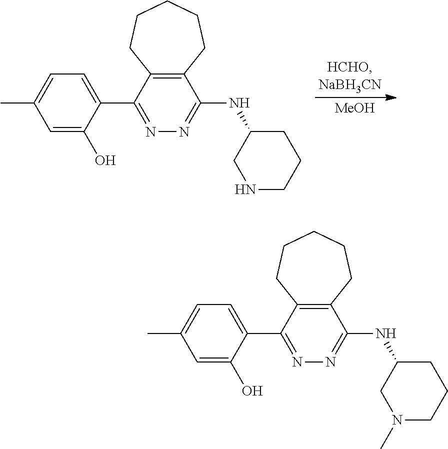

(R)-5-methyl-2-(4-(piperidin-3-ylamino)-6,7,8,9-tetrahydro-5H-cyclohepta[d]pyridazin-1-yl)phenol (380 mg, 1.08 mmol, 1.0 eq.) was dissolved in methanol (8 mL), then an aqueous formaldehyde solution (37%) (87 mg, 1.08 mmol, 1.0 eq.) and sodium cyanoborohydride (75 mg, 1.19 mmol, 1.1 eq.) were added, and the reaction solution was reacted at 25° C. for 20 min. After the materials reacted completely as monitored by TLC, the reaction solution was concentrated, and dichloromethane and saturated sodium bicarbonate were added, followed by liquid separation. The organic phase was dried, concentrated, and purified by preparative thin-layer chromatography (dichloromethane:methanol=7:1) to give the product (212 mg, yield: 54%).

¹H-NMR (400 MHz, DMSO-d6) δ (ppm): 9.49 (s, 1H), 7.99-7.97 (d, J=7.6 Hz, 1H), 6.73 (s, 1H), 6.68-6.66 (d, J=7.6 Hz, 1H), 6.29 (s, 1H), 4.50 (s, 1H), 3.68-3.51 (m, 1H), 3.45-3.43 (m, 1H), 3.26 (m, 1H), 2.74 (m, 6H), 2.48 (s, 1H), 2.27 (s, 3H), 2.08-1.89 (m, 2H), 1.77-1.76 (m, 4H), 1.52-1.46 (m, 5H).

Molecular formula: C₂₂H₃₀N₄O Precise molecular weight: 366.24 LC-MS (m/z): 367.17 [M+H]⁺.

### Example 75: Synthesis of (R)-2,3-difluoro-6-(4-((1-methylpiperidin-3-yl)amino)-5,6,7,8-tetrahydrophthalazin-1-yl)phenol (Compound 31a)

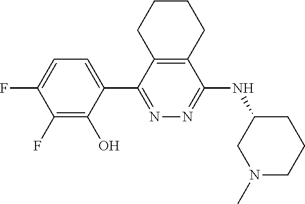 [SMILES: CN1CCC[C@@H](Nc2nnc(-c3ccc(F)c(F)c3O)c3c2CCCC3)C1]

### Step 1: Synthesis of Intermediate 1-bromo-2-(ethoxymethoxy)-3,4-difluorobenzene

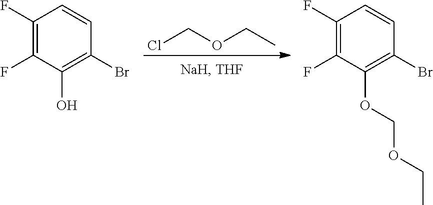

6-bromo-2,3-difluorophenol (1 g, 4.78 mmol, 1.0 eq.) was dissolved in anhydrous tetrahydrofuran (6 mL), 60% (mass fraction) NaH (287 mg, 7.17 mmol, 1.5 eq.) was added portionwise under an ice-water bath, with stirring for 30 min, followed by addition of chloromethyl ethyl ether (587 mg, 6.21 mmol, 1.3 eq.), and the reaction solution was stirred at room temperature for 2 h. After the reaction was completed as detected by TLC, water was added to the reaction solution to quench the reaction, followed by concentration, and then water (5 mL) and ethyl acetate (10 mL) were added, followed by liquid separation. The organic phase was washed successively with water (15 mL×2) and saturated brine (15 mL×2), dried, and concentrated to give the product (1.1 g, yield: 86.2%).

### Step 2: Synthesis of Intermediate (2-(ethoxymethoxy)-3,4-difluorophenyl)boric acid

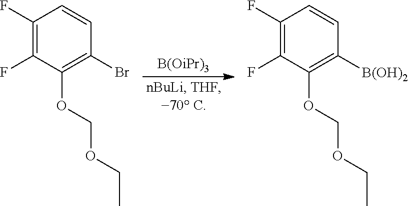 [SMILES: CCOCOc1c(Br)ccc(F)c1F ; "nBuLi, THF,-70° C." ; CCOCOc1c(B(O)O)ccc(F)c1F ; "B(OiPr)3"]

1-Bromo-2-(ethoxymethoxy)-3,4-difluorobenzene (1.06 g, 4 mmol, 1.0 eq.) was dissolved in anhydrous tetrahydrofuran (12 mL), and the reaction solution was cooled to −70° C. A 1.6 mol/L solution of n-butyllithium in n-hexane (3.8 mL, 6 mmol, 1.5 eq.) was added dropwise, with stirring for 2 h, then triisopropyl borate (978 mg, 5.2 mmol, 1.3 eq.) was added dropwise, and the reaction solution was stirred at room temperature for 3 h. After the reaction was completed as detected by TLC, water was added to the reaction solution to quench the reaction, followed by concentration. A 2 mol/L aqueous hydrochloric acid solution was added to adjust the pH to 6, followed by addition of water (5 mL) and ethyl acetate (10 mL), and liquid separation. The organic phase was dried and concentrated to give the product (750 mg, yield: 80.8%).

### Step 3: Synthesis of Intermediate (R)-4-(2-(ethoxymethoxy)-3,4-difluorophenyl)-N-(1-methylpiperidin-3-yl)-5,6,7,8-tetrahydrophthalazin-1-amine

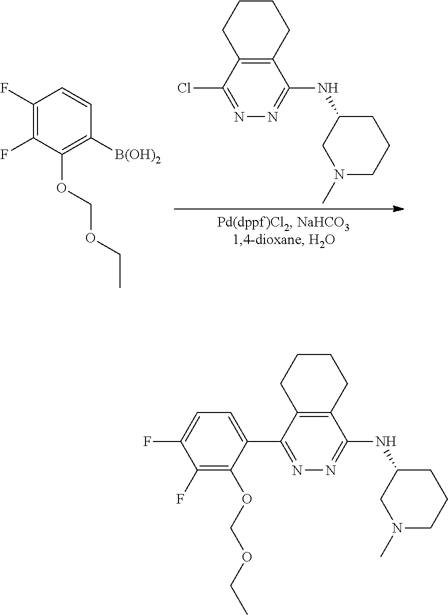 [SMILES: CCOCOc1c(B(O)O)ccc(F)c1F ; CN1CCC[C@@H](Nc2nnc(Cl)c3c2CCCC3)C1 ; CCOCOc1c(-c2nnc(N[C@@H]3CCCN(C)C3)c3c2CCCC3)ccc(F)c1F]

(R)-4-chloro-N-(1-methylpiperidin-3-yl)-5,6,7,8-tetrahydrophthalazin-1-amine (250 mg, 0.89 mmol, 1.0 eq.) was dissolved in 1,4-dioxane (2 mL) and water (1 mL), then (2-(ethoxymethoxy)-3,4-difluorophenyl)boronic acid (413 mg, 1.78 mmol, 2.0 eq.), sodium bicarbonate (150 mg, 1.78 mmol, 2.0 eq.) and Pd(dppf)Cl₂ (65 mg, 0.089 mmol, 0.1 eq.) were added successively, and the reaction solution was stirred at 110° C. overnight in nitrogen atmosphere. After there remained a small number of materials as detected by TLC, the reaction solution was concentrated, followed by addition of water (5 mL) and extraction with dichloromethane (5 mL×3). The organic phase was dried and filtered under vacuum, the filtrate was concentrated, and the crude product was purified by silica gel column chromatography (dichloromethane:methanol=20:1 to 10:1) to give the product (80 mg, yield: 20.8%).

### Step 4: Synthesis of compound (R)-2,3-difluoro-6-(4-((1-methylpiperidin-3-yl)amino)-5,6,7,8-tetrahydrophthalazin-1-yl)phenol

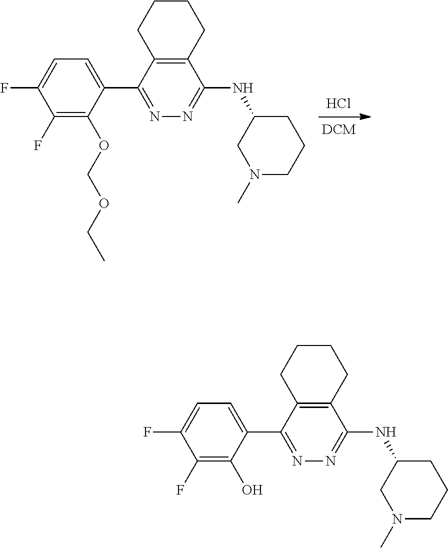 [SMILES: CCOCOc1c(-c2nnc(N[C@@H]3CCCN(C)C3)c3c2CCCC3)ccc(F)c1F ; Cl ; CN1CCC[C@@H](Nc2nnc(-c3ccc(F)c(F)c3O)c3c2CCCC3)C1]

(R)-4-(2-(ethoxymethoxy)-3,4-difluorophenyl)-N-(1-methylpiperidin-3-yl)-5,6,7,8-tetrahydrophthalazin-1-amine (80 mg, 0.19 mmol, 1.0 eq.) was dissolved in dichloromethane (1 mL), then a 4 mol/L solution of hydrogen chloride in 1,4-dioxane (0.23 mL, 5.0 eq.) was added, and the reaction solution was stirred at room temperature for 3 h. After the reaction was completed as detected by TLC, the reaction solution was poured into water, and the pH was adjusted to 8 with sodium bicarbonate, followed by liquid separation and extraction with dichloromethane (5 mL×2). The organic phase was dried, filtered under vacuum, and concentrated, and the crude product was purified by preparative thin-layer chromatography to give the product (37 mg, yield: 53.4%).

¹H-NMR (400 MHz, DMSO-d6) δ (ppm): 10.47 (s, 1H), 7.00-6.89 (m, 2H), 5.82 (s, 1H), 4.38 (s, 1H), 3.15 (s, 1H), 2.87 (s, 1H), 2.77 (s, 1H), 2.40-2.31 (m, 6H), 1.85-1.60 (m, 10H).

Molecular formula: C₂₀H₂₄F₂N₄O Precise molecular weight: 374.19 LC-MS (m/z): 375.26 [M+H]⁺.

### Example 76: Synthesis of (R)-6-methyl-3-(4-((1-methylpiperidin-3-yl)amino)phthalazin-1-yl)pyridin-2-ol (Compound 106) Referring to the Aforementioned

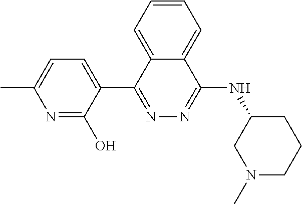 [SMILES: Cc1ccc(-c2nnc(N[C@@H]3CCCN(C)C3)c3ccccc23)c(O)n1]

¹HNMR (300 MHz, DMSO-d6) δ (ppm): 11.95 (s, 1H), 8.50 (s, 1H), 7.89-7.77 (m, 2H), 7.60-7.52 (m, 3H), 6.19 (d, J=6.9 Hz, 1H), 4.74 (s, 1H), 3.63-3.59 (m, 2H), 3.17 (s, 1H), 2.78 (s, 4H), 2.30 (s, 3H), 2.08 (s, 1H), 1.84 (s, 3H).

Molecular formula: C₂₀H₂₃N₅O Precise molecular weight: 349.19 LC-MS (m/z): 350.28 [M+H]⁺.

### Example 77: Synthesis of (R)-4,5-dimethyl-2-(4-((1-methylpiperidin-3-yl)amino)phthalazin-1-yl)phenol (Compound 35) Referring to the Aforementioned Examples

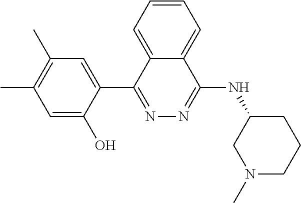 [SMILES: Cc1cc(O)c(-c2nnc(N[C@@H]3CCCN(C)C3)c3ccccc23)cc1C]

¹HNMR (300 MHz, DMSO-d6) δ (ppm): 9.37 (s, 1H), 8.41 (d, J=8.1 Hz, 1H), 7.86-7.74 (m, 2H), 7.54 (d, J=7.8 Hz, 1H), 7.14 (s, 1H), 7.04 (s, 1H), 6.80 (s, 1H), 4.49 (s, 1H), 3.21 (d, J=8.4 Hz, 1H), 2.84 (d, J=11.1 Hz, 1H), 2.35 (s, 3H), 2.23-2.18 (m, 8H), 2.02-1.97 (m, 1H), 7.84-1.80 (m, 1H), 1.72-1.49 (m, 2H).

Molecular formula: C₂₂H₂₆N₄O Precise molecular weight: 362.21 LC-MS (m/z): 363.34 [M+H]⁺.

### Example 78: Synthesis of (R)-5-methyl-2-(4-((1-methylpiperidin-3-yl)amino)furo[3,4-d]pyridazin-1-yl)phenol (Compound 44) Referring to the Aforementioned Examples

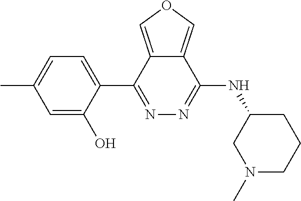 [SMILES: Cc1ccc(-c2nnc(N[C@@H]3CCCN(C)C3)c3cocc23)c(O)c1]

¹HNMR (300 MHz, DMSO-d6) δ (ppm): 15.0 (s, 1H), 9.20 (d, J=1.2 Hz, 1H), 8.96 (s, 2H), 8.03 (s, 1H), 7.82 (d, J=8.4 Hz, 1H), 6.77 (d, J=5.7 Hz, 1H), 4.47 (s, 1H), 3.34 (d, J=8.4 Hz, 3H), 3.01 (d, J=11.7 Hz, 1H), 2.54 (s, 3H), 2.31 (s, 3H), 1.95-1.89 (m, 2H), 1.75-1.59 (m, 2H).

Molecular formula: C₁₉H₂₂N₄O₂ Precise molecular weight: 338.17 LC-MS (m/z): 339.15 [M+H]⁺.

### Example 79: Synthesis of (R)-3-hydroxy-4-(4-((1-methylpiperidin-3-yl)amino)thieno[3,4-d]pyridazin-1-yl)benzonitrile (Compound 58) Referring to the Aforementioned Examples

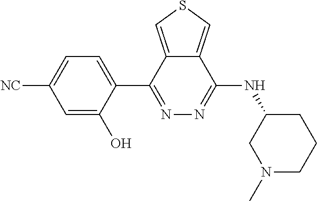 [SMILES: CN1CCC[C@@H](Nc2nnc(-c3ccc(C#N)cc3O)c3cscc23)C1]

¹HNMR (300 MHz, DMSO-d6) δ (ppm): 9.00 (s, 1H), 8.58 (d, J=2.7 Hz, 1H), 8.05 (d, J=8.1 Hz, 2H), 7.42-7.38 (m, 2H), 4.62 (s, 1H), 3.18 (s, 2H), 2.79-2.73 (m, 1H), 2.68 (s, 3H), 2.04-1.99 (m, 3H), 1.81-1.77 (m, 2H).

Molecular formula: C₁₉H₁₉N₅OS Precise molecular weight: 365.13 LC-MS (m/z): 366.14[M+H]⁺

### Example 80: Synthesis of (R)-2-methyl-5-(4-((1-methylpiperidin-3-yl)amino)phthalazin-1-yl)pyridin-4-ol (Compound 107) Referring to the Aforementioned Examples

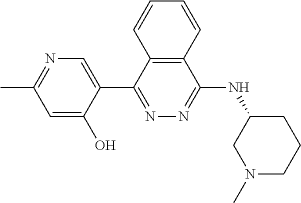 [SMILES: Cc1cc(O)c(-c2nnc(N[C@@H]3CCCN(C)C3)c3ccccc23)cn1]

¹HNMR (300 MHz, DMSO-d6) δ (ppm): 8.30 (d, J=7.5 Hz, 1H), 7.94 (s, 1H), 7.90-7.83 (m, 2H), 7.73 (d, J=7.8 Hz, 1H), 6.51 (s, 1H), 4.63 (s, 1H), 3.78 (s, 3H), 3.43 (d, J=9.6 Hz, 1H), 3.09 (s, 2H), 2.90 (s, 3H), 2.47 (s, 3H), 2.15 (s, 2H), 1.91 (s, 2H).

Molecular formula: C₂₀H₂₃N₅O Precise molecular weight: 349.19 LC-MS (m/z): 350.12 [M+H]⁺.

### Example 81: Synthesis of 5-(1-hydroxyethyl)-2-(4-(((R)-1-methylpiperidin-3-yl)amino)phthalazin-1-yl)phenol (Compound 92) Referring to the Aforementioned Examples

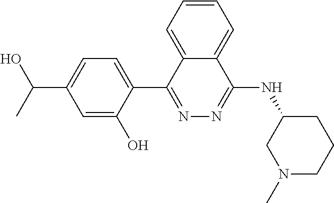 [SMILES: CC(O)c1ccc(-c2nnc(N[C@@H]3CCCN(C)C3)c3ccccc23)c(O)c1]

¹HNMR (400 MHz, DMSO-d6) δ (ppm): 9.75 (s, 1H), 8.53 (s, 1H), 7.91-7.83 (m, 2H), 7.55 (d, J=8.0 Hz, 1H), 7.46-7.35 (m, 3H), 7.23 (d, J=7.6 Hz, 2H), 7.05 (s, 1H), 6.94-6.91 (m, 1H), 5.26 (d, J=3.2 Hz, 1H), 4.75-4.73 (m, 2H), 3.72 (s, 1H), 2.94 (s, 2H), 2.81 (s, 3H), 2.20-1.78 (m, 4H), 1.38 (d, J=6.4 Hz, 3H).

Molecular formula: C₂₂H₂₆N₄O₂ Precise molecular weight: 378.21 LC-MS (m/z): 379.16 [M+H]⁺.

### Example 82: Synthesis of (R)-5-(2-hydroxypropan-2-yl)-2-(4-((1-methylpiperidin-3-yl)amino)phthalazin-1-yl)phenol (Compound 94) Referring to the Aforementioned Examples

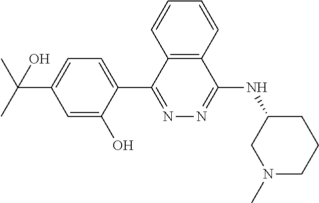 [SMILES: CN1CCC[C@@H](Nc2nnc(-c3ccc(C(C)(C)O)cc3O)c3ccccc23)C1]

¹HNMR (400 MHz, DMSO-d6) δ (ppm): 9.61 (s, 1H), 8.44 (d, J=6.8 Hz, 1H), 7.88-7.78 (m, 2H), 7.53 (d, J=8.0 Hz, 1H), 7.28 (s, 1H), 7.21 (d, J=8.0 Hz, 1H), 7.15 (d, J=1.2 Hz, 1H), 7.03-7.01 (m, 1H), 5.07 (s, 1H), 4.57 (s, 1H), 3.02 (s, 2H), 2.01-1.89 (m, 3H), 1.74-1.63 (m, 3H), 1.47 (s, 6H).

Molecular formula: C₂₃H₂₈N₄O₂ Precise molecular weight: 392.22 LC-MS (m/z): 393.13 [M+H]⁺.

### Example 83: Synthesis of (R)-5-hydroxy-6-(4-((1-methylpiperidin-3-yl)amino)phthalazin-1-yl)nicotinonitrile (Compound 109) Referring to the Aforementioned Examples

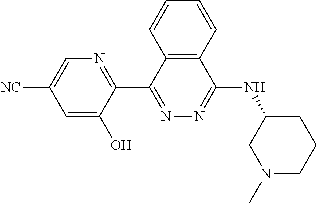 [SMILES: CN1CCC[C@@H](Nc2nnc(-c3ncc(C#N)cc3O)c3ccccc23)C1]

¹HNMR (400 MHz, DMSO-d6) δ (ppm): 15.41 (s, 1H), 9.46 (d, J=6.0 Hz, 1H), 8.57 (s, 1H), 8.49 (d, J=6.4 Hz, 1H), 7.96 (s, 2H), 7.83 (s, 1H), 7.71 (d, J=6.8 Hz, 1H), 4.42 (s, 1H), 3.05 (d, J=8.0 Hz, 1H), 2.72 (d, J=10.0 Hz, 1H), 2.22 (s, 3H), 1.99-1.92 (m, 3H), 1.77-1.74 (m, 1H), 1.63-1.48 (m, 2H).

Molecular formula: C₂₀H₂₀N₆O Precise molecular weight: 360.17 LC-MS (m/z): 361.09 [M+H]⁺.

### Example 84: Synthesis of (R)-4-(4-((1-methylpiperidin-3-yl)amino)phthalazin-1-yl)pyridin-3-ol (Compound 115) Referring to the Aforementioned Examples

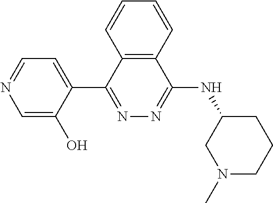 [SMILES: CN1CCC[C@@H](Nc2nnc(-c3ccncc3O)c3ccccc23)C1]

¹HNMR (400 MHz, DMSO-d6) δ (ppm): 10.22 (s, 1H), 8.58 (s, 1H), 8.40 (s, 1H), 8.21 (d, J=4.8 Hz, 1H), 7.92-7.88 (m, 1H), 7.85-7.81 (m, 1H), 7.67 (s, 1H), 7.47 (d, J=8.0 Hz, 1H), 7.47 (d, J=4.8 Hz, 1H), 4.75 (s, 1H), 3.56 (s, 2H), 3.23 (s, 1H), 2.74 (s, 4H), 2.01 (s, 2H), 1.83 (s, 2H).

Molecular formula: C₁₉H₂₁N₅O Precise molecular weight: 335.17 LC-MS (m/z): 336.12 [M+H]⁺.

### Example 85: Synthesis of (R)-2-(4-((1-methylpiperidin-3-yl)amino)phthalazin-1-yl)-5-(oxazol-2-yl)phenol (Compound 119) Referring to the Aforementioned Examples

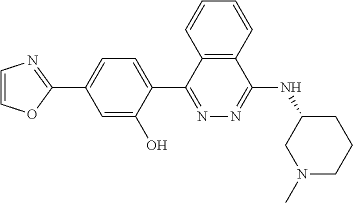 [SMILES: CN1CCC[C@@H](Nc2nnc(-c3ccc(-c4ncco4)cc3O)c3ccccc23)C1]

¹HNMR (400 MHz, DMSO-d6) δ (ppm): 11.18 (d, J=5.2 Hz, 1H), 10.24 (s, 1H), 8.59 (s, 1H), 7.88-7.86 (m, 1H), 7.82-7.78 (m, 2H), 7.62 (s, 2H), 7.46 (d, J=7.6 Hz, 1H), 7.04-7.01 (m, 1H), 6.53 (d, J=7.2 Hz, 1H), 4.75 (s, 1H), 3.51 (s, 2H), 3.17 (s, 1H), 2.69 (s, 4H), 2.00 (s, 2H), 1.82 (s, 2H).

Molecular formula: C₂₃H₂₃N₅O₂ Precise molecular weight: 401.19 LC-MS (m/z): 402.13 [M+H]⁺.

### Example 86: Synthesis of (R)-3-hydroxy-4-(4-((1-(oxetan-3-yl)piperidin-3-yl)amino)phthalazin-1-yl)benzonitrile (Compound 128) Referring to the Aforementioned Examples

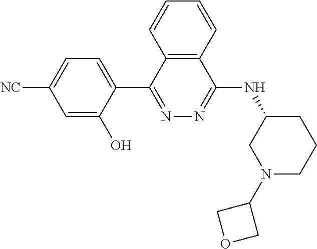 [SMILES: N#Cc1ccc(-c2nnc(N[C@@H]3CCCN(C4COC4)C3)c3ccccc23)c(O)c1]

¹HNMR (400 MHz, DMSO-d6) δ (ppm): 10.48 (s, 1H), 8.39 (d, J=8.0 Hz, 1H), 7.88-7.84 (m, 1H), 7.81-7.77 (m, 1H), 7.50 (d, J=7.6 Hz, 1H), 7.43-7.41 (m, 1H), 7.33 (d, J=1.2 Hz, 1H), 7.19 (d, J=7.6 Hz, 1H), 4.56-4.52 (m, 2H), 4.49-4.44 (m, 3H), 3.48-3.45 (m, 1H), 3.04-3.01 (m, 1H), 2.67 (d, J=10.8 Hz, 1H), 2.05 (d, J=8.8 Hz, 1H), 1.88-1.77 (m, 3H), 1.67-1.61 (m, 1H), 1.55-1.49 (m, 1H).

Molecular formula: C₂₃H₂₃N₅O₂ Precise molecular weight: 401.19 LC-MS (m/z): 402.14 [M+H]⁺.

### Example 87: Synthesis of (R)-5-methyl-2-(5-((1-methylpiperidin-3-yl)amino)-pyrido[2,3-d]pyridazin-8-yl)phenol (Compound 37) Referring to the Aforementioned Examples

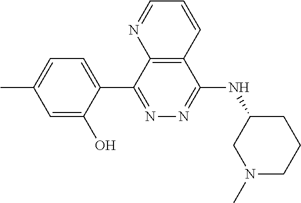 [SMILES: Cc1ccc(-c2nnc(N[C@@H]3CCCN(C)C3)c3cccnc23)c(O)c1]

¹H-NMR (300 MHz, DMSO-d6) δ (ppm): 11.92 (brs, 1H), 9.20-9.22 (m, 1H), 9.01 (s, 1H), 8.18-8.21 (d, 1H), 7.95-7.99 (m, 1H), 7.84 (s, 1H), 6.77 (s, 1H), 6.73 (s, 1H), 4.66 (s, 1H), 3.55 (s, 3H), 2.72 (s, 4H), 2.31 (s, 3H), 1.99-2.02 (d, 2H), 1.79-1.83 (d, 2H).

Molecular formula: C₂₀H₂₃N₅O Precise molecular weight: 349.19 LC-MS (m/z): 350.22 [M+H]⁺.

### Example 88: Synthesis of (R)-5-methyl-2-(8-methyl-4-((1-methylpiperidin-3-yl)amino)phthalazin-1-yl)phenol (Compound 2) Referring to the Aforementioned Examples

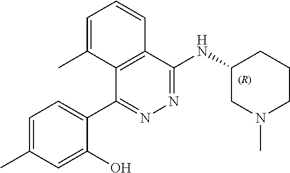

¹HNMR (400 MHz, DMSO-d6) δ (ppm): 9.31 (s, 1H), 8.31 (s, 1H), 7.74-7.72 (d, 1H), 7.60-7.58 (d, 1H), 7.31 (s, 1H), 7.06-7.03 (m, 1H), 6.73 (s, 2H), 4.68 (s, 1H), 2.57 (s, 4H), 2.50 (s, 3H), 2.31 (s, 3H), 2.06-1.99 (t, 5H), 1.83-1.81 (m, 2H).

The two-dimensional NOE spectrum showed coupling signals between 7.60-7.58 (d, 1H) and 7.74-7.72 (d, 1H) and coupling signals between 7.31 (s, 1H) and 8.31 (s, 1H).

Molecular formula: C₂₁H₂₆N₄O Precise molecular weight: 362.21 LC-MS (m/z): 363.26 [M+H]⁺.

### Example 89: Synthesis of (R)-5-methyl-2-(7-((1-methylpiperidin-3-yl)amino)-1H-imidazo[4,5-d]pyridazin-4-yl)phenol (Compound 134) Referring to the Aforementioned Examples

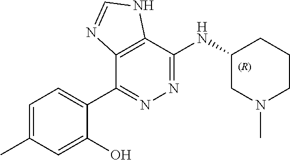

¹HNMR (300 MHz, DMSO-d6) δ (ppm): 9.03 (s, 1H), 8.60 (s, 1H), 7.45-7.43 (d, 1H), 6.79-6.77 (d, 2H), 4.44 (s, 1H), 3.33 (s, 3H), 2.95 (s, 1H), 2.66 (s, 2H), 2.55 (s, 3H), 2.31 (s, 3H), 1.97-1.92 (d, 2H), 1.75-1.59 (m, 2H).

Molecular formula: C₁₈H₂₂N₆O Precise molecular weight: 338.19 LC-MS (m/z): 339.27 [M+H]⁺.

### Example 90: Synthesis of (R)-5-methyl-2-(4-((1-methylpiperidin-3-yl)amino)-6-(methylsulfonyl)phthalazin-1-yl)phenol (Compound 82) Referring to the Aforementioned Examples

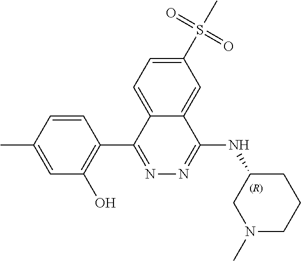

¹HNMR (300 MHz, DMSO-d6) δ (ppm): 9.66 (s, 1H), 9.11 (s, 1H), 8.29-8.26 (d, 1H), 7.72-7.70 (d, 1H), 7.20-7.17 (d, 1H), 6.85-6.79 (t, 2H), 4.73 (s, 1H), 3.38 (s, 5H), 2.80 (s, 3H), 2.55 (s, 2H), 2.34 (s, 3H), 2.08-2.02 (t, 2H), 1.88-1.84 (d, 2H).

Molecular formula: C₂₂H₂₆N₄O₃S Precise molecular weight: 426.17 LC-MS (m/z): 427.13 [M+H]⁺.

### Example 91: Synthesis of (R)-2-(6-(furan-2-yl)-4-((1-methylpiperidin-3-yl)amino)phthalazin-1-yl)-5-methylphenol (Compound 83) Referring to the Aforementioned Examples

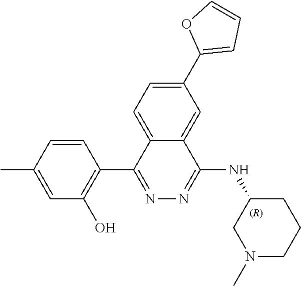

¹HNMR (300 MHz, CDCl₃) δ(ppm): 8.43 (s, 1H), 8.28-8.25 (d, 1H), 8.08-8.05 (d, 1H), 7.62-7.59 (d, 1H), 7.51-7.48 (d, 1H), 7.09-7.02 (d, 2H), 6.85-6.83 (d, 1H), 6.60 (s, 2H), 4.79 (s, 1H), 2.95-2.93 (d, 2H), 2.68 (s, 1H), 2.44-2.42 (d, 6H), 2.14 (s, 1H), 2.00 (s, 2H), 1.73 (s, 2H).

Molecular formula: C₂₅H₂₆N₄O₂ Precise molecular weight: 414.21 LC-MS (m/z): 415.21 [M+H]⁺.

### Example 92: Synthesis of (R)-2-(6-(dimethylamino)-4-((1-methylpiperidin-3-yl)amino)phthalazin-1-yl)-5-methylphenol (Compound 84) Referring to the Aforementioned Examples

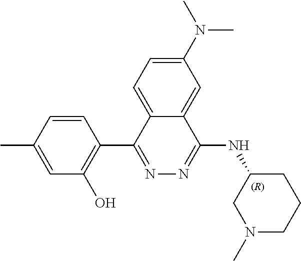

¹HNMR (300 MHz, DMSO-d6) δ (ppm): 7.51-7.48 (d, 1H), 7.37-7.34 (d, 2H), 7.23-7.21 (d, 1H), 6.85 (s, 1H), 6.80-6.78 (d, 1H), 4.57 (s, 1H), 3.50-3.42 (d, 2H), 2.44-3.42 (m, 2H), 3.41-3.40 (d, 1H), 3.16 (s, 6H), 3.06 (s, 1H), 2.57 (s, 3H), 2.34 (s, 3H), 2.02-1.99 (t, 2H), 1.78-1.74 (d, 2H).

Molecular formula: C₂₃H₂₉N₅O Precise molecular weight: 391.24 LC-MS (m/z): 392.19 [M+H]⁺.

### Example 93: Synthesis of 3,5-difluoro-2-(4-(((R)-1-methylpiperidin-3-yl)amino)-5,6,7,8-tetrahydrophthalazin-1-yl)phenol (Compound 28a) Referring to the Aforementioned Examples

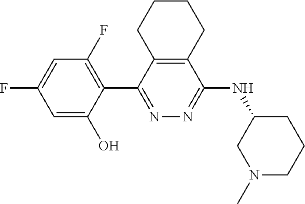 [SMILES: CN1CCC[C@@H](Nc2nnc(-c3c(O)cc(F)cc3F)c3c2CCCC3)C1]

¹H-NMR (400 MHz, DMSO-d6) δ (ppm): 10.48 (s, 1H), 6.73 (t, J=9.04 Hz, 1H), 6.61 (d, J=10.4 Hz, 1H), 5.77 (s, 1H), 4.33 (s, 1H), 3.07 (s, 1H), 2.77 (s, 1H), 2.37-2.35 (m, 7H), 1.84-1.51 (m, 10H).

Molecular formula: C₂₀H₂₄F₂N₄O Precise molecular weight: 374.19 LC-MS (m/z): 375.16 [M+H]⁺.

### Example 94: Synthesis of (R)-2-(4-((1-methylpiperidin-3-yl)amino)-5,6,7,8-tetrahydrophthalazin-1-yl)phenol (Compound 25a) Referring to the Aforementioned Examples

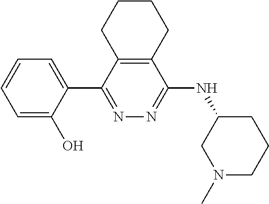 [SMILES: CN1CCC[C@@H](Nc2nnc(-c3ccccc3O)c3c2CCCC3)C1]

¹H-NMR (400 MHz, DMSO-d6) δ (ppm): 9.69 (s, 1H), 7.24-7.20 (m, 1H), 7.12-7.09 (m, 1H), 6.92 (d, J=8.0 Hz, 1H), 6.80 (t, J=7.4 Hz, 1H), 5.77 (s, 1H), 4.41 (s, 1H), 3.21 (s, 1H), 3.04-2.92 (m, 2H), 2.48-2.33 (m, 8H), 1.87-1.58 (m, 8H).

Molecular formula: C₂₀H₂₆N₄O Precise molecular weight: 338.21 LC-MS (m/z): 339.12 [M+H]⁺.

### Example 95: Synthesis of (R)-4-fluoro-5-methyl-2-(4-((1-methylpiperidin-3-yl)amino)-5,6,7,8-tetrahydrophthalazin-1-yl)phenol (Compound 27a) Referring to the Aforementioned Examples

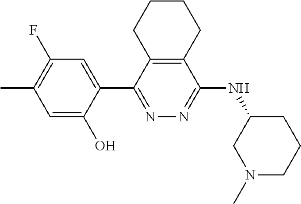 [SMILES: Cc1cc(O)c(-c2nnc(N[C@@H]3CCCN(C)C3)c3c2CCCC3)cc1F]

¹H-NMR (400 MHz, DMSO-d6) δ (ppm): 9.60 (s, 1H), 6.86 (d, J=9.84 Hz, 1H), 6.77 (d, J=6.76 Hz, 1H), 5.87 (s, 1H), 4.42 (s, 1H), 3.31-3.20 (m, 2H), 3.16 (s, 1H), 2.99 (s, 1H), 2.40-2.35 (m, 5H), 2.20 (s, 3H), 1.88-1.59 (m, 10H).

Molecular formula: C₂₁H₂₇FN₄O Precise molecular weight: 370.22 LC-MS (m/z): 371.21 [M+H]⁺.

### Example 96: Synthesis of (R)-4,5-difluoro-2-(4-((1-methylpiperidin-3-yl)amino)-5,6,7,8-tetrahydrophthalazin-1-yl)phenol (Compound 30a) Referring to the Aforementioned Examples

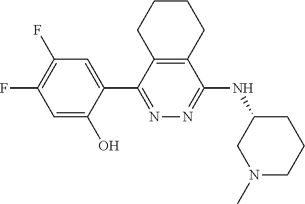 [SMILES: CN1CCC[C@@H](Nc2nnc(-c3cc(F)c(F)cc3O)c3c2CCCC3)C1]

¹H-NMR (400 MHz, DMSO-d6) δ (ppm): 10.61 (s, 1H), 6.99-6.94 (m, 1H), 6.91-6.87 (m, 1H), 5.85 (s, 1H), 4.39 (s, 1H), 3.13 (s, 1H), 2.85 (s, 1H), 2.41-2.28 (m, 7H), 1.85-1.62 (m, 10H).

Molecular formula: C₂₀H₂₄F₂N₄O Precise molecular weight: 374.19 LC-MS (m/z): 375.22 [M+H]⁺.

### Example 97: Synthesis of (R)-4-(2-amino-4-methylphenyl)-N-(1-methylpiperidin-3-yl)phthalazin-1-amine (Compound 39) Referring to the Aforementioned Examples

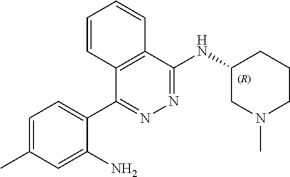

¹HNMR (300 MHz, DMSO-d6) δ (ppm): 8.40-8.37 (d, J=9 Hz, 1H), 7.87-7.82 (m, 2H), 7.58-7.55 (d, J=9 Hz, 1H), 7.05-7.02 (d, J=9 Hz, 1H), 6.96-6.93 (d, J=9 Hz, 1H), 6.65 (s, 1H), 6.53-6.50 (d, J=9 Hz, 1H), 4.89 (s, 2H), 4.43-4.41 (d, J=6 Hz, 1H), 3.10-3.08 (d, J=6 Hz, 1H), 2.73-2.70 (d, J=9 Hz, 1H), 2.27 (s, 3H), 2.22 (s, 3H), 1.99-1.40 (m, 6H).

Molecular formula: C₂₁ H₂₅N₅ Precise molecular weight: 347.47 LC-MS (m/z): 348.25 [M+H]⁺.

### Example 98: Synthesis of (R)-4-(4-methyl-2-(methylamino)phenyl)-N-(1-methylpiperidin-3-yl)phthalazin-1-amine (Compound 40) Referring to the Aforementioned Examples

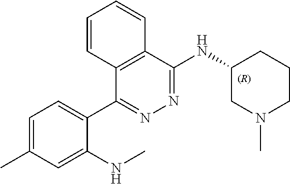

¹HNMR (300 MHz, DMSO-d6) δ (ppm): 8.45-8.42 (d, J=9 Hz, 1H), 7.88-7.75 (m, 2H), 7.51-7.49 (d, J=6 Hz, 1H), 7.24 (s, 1H), 6.96-6.93 (d, J=9 Hz, 1H), 6.56-6.53 (m, 2H), 5.03-5.02 (d, J=3 Hz, 1H), 4.56 (s, 1H), 2.98-2.95 (d, J=9 Hz, 1H), 2.63-2.62 (d, J=3 Hz, 3H), 2.46 (s, 3H), 2.34 (s, 3H), 2.28-1.60 (m, 6H).

Molecular formula: C₂₂H₂₇N₅ Precise molecular weight: 361.49 LC-MS (m/z): 362.26 [M+H]⁺.

### Example 99: Synthesis of (R)-5-methyl-2-(4-((1-methylpiperidin-3-yl)amino)phthalazin-1-yl)phenol (Compound 41) Referring to the Aforementioned Examples

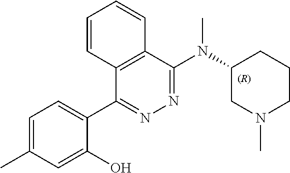

¹HNMR (300 MHz, DMSO-d6) δ (ppm): 9.63 (s, 1H), 8.48 (s, 1H), 7.89-7.77 (m, 2H), 7.54-7.52 (m, 1H), 7.41 (s, 1H), 7.17-7.15 (m, 1H), 6.82-6.76 (m, 2H), 4.65 (s, 1H), 3.47-3.36 (m, 2H), 3.17-3.12 (m, 2H), 2.51-2.50 (m, 3H), 2.33 (s, 3H), 1.99-1.77 (m, 4H).

Molecular formula: C₂₂H₂₆N₄O Precise molecular weight: 362.48 LC-MS (m/z): 363.33 [M+H]⁺.

### Example 100: Synthesis of (R)—N-(5-methyl-2-(4-((1-methylpiperidin-3-yl)amino)phthalazin-1-yl)phenyl)acetamide (Compound 43) Referring to the Aforementioned Examples

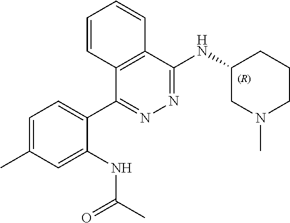

¹HNMR (300 MHz, DMSO-d6) δ (ppm): 9.19 (s, 1H), 8.41-8.38 (m, 1H), 7.86-7.64 (m, 3H), 7.39-7.36 (m, 1H), 7.29-7.26 (m, 1H), 7.12-7.10 (m, 2H), 4.45-4.43 (m, 1H), 3.14-3.11 (m, 1H), 2.79-2.77 (m, 1H), 2.39 (s, 3H), 2.25 (s, 3H), 2.01-1.91 (m, 3H), 1.80-1.54 (m, 6H).

Molecular formula: C₂₃H₂₇N₅O Precise molecular weight: 389.22 LC-MS (m/z): 390.30 [M+H]⁺.

### Example 101: Synthesis of (R)-5-methyl-2-(4-((1-methylpiperidin-3-yl)amino)phthalazin-1-yl)benzamide (Compound 76) Referring to the Aforementioned Examples

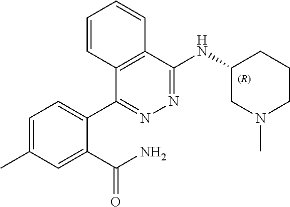

¹HNMR (300 MHz, DMSO-d6) δ (ppm): 8.51 (s, 1H), 7.86-7.68 (m, 3H), 7.53 (s, 1H), 7.46-7.06 (m, 4H), 4.63 (s, 1H), 3.02 (s, 1H), 2.56 (s, 3H), 2.37 (s, 3H), 2.04-1.34 (m, 7H).

Molecular formula: C₂₂H₂₅N₅O Precise molecular weight: 375.21 LC-MS (m/z): 376.22 [M+H]⁺.

### Example 102: Synthesis of (R)-1-(3-((4-(2-hydroxy-4-methylphenyl)phthalazin-1-yl)amino)piperidin-1-yl)prop-2-en-1-one (Compound 85) Referring to the Aforementioned Examples

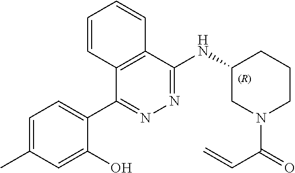

¹HNMR (300 MHz, DMSO-d6) δ (ppm): 9.61 (s, 1H), 8.40-8.38 (m, 1H), 7.88-7.76 (m, 2H), 7.54-7.52 (m, 1H), 7.19-7.17 (m, 2H), 6.81-6.76 (m, 2H), 6.14-6.05 (m, 1H), 5.70-5.59 (m, 1H), 4.70-4.00 (m, 3H), 3.35-2.71 (m, 2H), 2.33 (s, 3H), 2.15-1.24 (m, 5H).

Molecular formula: C₂₃H₂₄N₄O₂ Precise molecular weight: 388.19 LC-MS (m/z): 389.12 [M+H]⁺.

### Example 103: Synthesis of (R)-3-hydroxy-N-methyl-4-(4-((1-methylpiperidin-3-yl)amino)phthalazin-1-yl)benzamide (Compound 86) Referring to the Aforementioned Examples

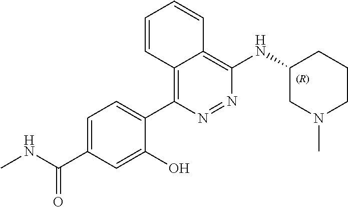

¹HNMR (300 MHz, DMSO-d6) δ (ppm): 9.99 (s, 1H), 8.51-8.47 (m, 2H), 7.90-7.78 (m, 2H), 7.66 (s, 1H), 7.49-7.34 (m, 4H), 4.76 (s, 1H), 3.56 (s, 1H), 3.24-3.09 (m, 2H), 2.89-2.75 (m, 6H), 2.13-1.80 (m, 5H).

Molecular formula: C₂₂H₂₅N₅O₂ Precise molecular weight: 391.20 LC-MS (m/z): 392.14 [M+H]⁺.

### Example 104: Synthesis of (R)-2-(4-((1-methylpiperidin-3-yl)amino)phthalazin-1-yl)-5-morpholinophenol (Compound 91) Referring to the Aforementioned Examples

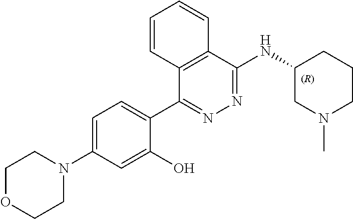

¹HNMR (400 MHz, DMSO-d6) δ (ppm): 9.96 (s, 1H), 8.47 (s, 1H), 7.89-7.79 (m, 2H), 7.67-7.65 (d, J=8 Hz, 1H), 7.37 (s, 1H), 7.21-7.19 (d, J=8 Hz, 1H), 6.59-6.53 (m, 2H), 4.63 (s, 1H), 3.49 (s, 3H), 3.16-3.14 (m, 5H), 2.65 (s, 3H), 2.03-1.77 (m, 4H).

Molecular formula: C₂₅H₂₉N₅O₂ Precise molecular weight: 419.23 LC-MS (m/z): 420.19 [M+H]⁺.

### Example 105: Synthesis of (R)-4-(4-((1-(cyclopropylmethyl)piperidin-3-yl)amino)-5,6,7,8-tetrahydrophthalazin-1-yl)-3-hydroxybenzonitrile (Compound 11a) Referring to the Aforementioned Examples

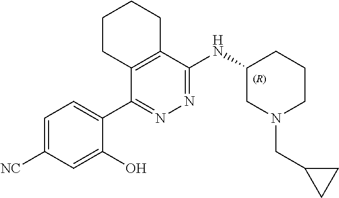

¹HNMR (400 MHz, DMSO-d6) δ (ppm): 10.53 (s, 1H), 7.35-7.24 (m, 3H), 5.69-5.68 (m, 1H), 4.29 (s, 1H), 3.09 (s, 1H), 2.83 (s, 1H), 2.37-2.09 (m, 9H), 1.86-1.70 (m, 4H), 1.61-1.46 (m, 4H), 0.87-0.84 (m, 2H), 0.47-0.44 (m, 2H).

Molecular formula: C₂₄H₂₉N₅O Precise molecular weight: 403.24 LC-MS (m/z): 404.15 [M+H]⁺.

### Example 106: Synthesis of (R)-4-(4-((1-acetylpiperidin-3-yl)amino)-5,6,7,8-tetrahydrophthalazin-1-yl)-3-hydroxybenzonitrile (Compound 12a) Referring to the Aforementioned Examples

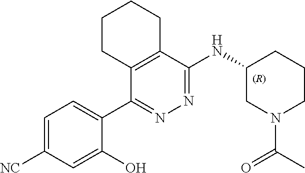

¹HNMR (400 MHz, DMSO-d6) δ (ppm): 10.50 (s, 1H), 7.33 (s, 2H), 7.24 (s, 1H), 5.89-5.77 (m, 1H), 4.49-4.10 (m, 3H), 3.04-2.90 (m, 1H), 2.67-2.59 (m, 1H), 2.41-2.28 (m, 4H), 2.02-2.01 (m, 4H), 1.76-1.60 (m, 7H).

Molecular formula: C₂₂H₂₅N₅O₂ Precise molecular weight: 391.20 LC-MS (m/z): 392.11 [M+H]⁺.

### Example 107: Synthesis of (R)-2-(4-((1-methylpiperidin-3-yl)amino)phthalazin-1-yl)-5-(methylsulfonyl)phenol (Compound 75) Referring to the Aforementioned Examples

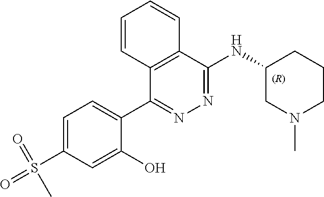

¹HNMR (400 MHz, DMSO-d6) δ (ppm): 11.06 (s, 1H), 7.80-7.71 (m, 3H), 7.59-7.57 (m, 1H), 7.30-7.28 (m, 1H), 6.94-6.81 (m, 2H), 4.70 (s, 1H), 3.68-6.65 (m, 1H), 3.17 (s, 3H), 2.68 (s, 3H), 2.42-2.39 (m, 2H), 2.16-2.12 (m, 2H), 1.60-1.53 (m, 2H).

Molecular formula: C₂₁H₂₄N₄O₃S Precise molecular weight: 412.16 LC-MS (m/z): 413.09 [M+H]⁺.

### Example 108: Synthesis of (R)-4-(4-((1-(cyclopropylsulfonyl)piperidin-3-yl)amino)phthalazin-1-yl)-3-hydroxybenzonitrile (Compound 103) Referring to the Aforementioned Examples

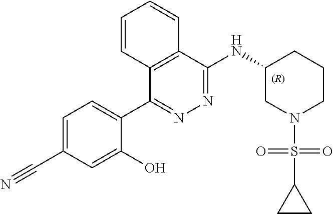

¹HNMR (400 MHz, DMSO-d6) δ (ppm): 10.47 (s, 1H), 8.42-8.40 (d, J=8 Hz, 1H), 7.89 (t, J=8 Hz, 1H), 7.81 (t, J=8 Hz, 1H), 7.52-7.50 (d, J=8 Hz, 1H), 7.44-7.42 (d, J=8 Hz, 2H), 7.34-7.33 (m, 2H), 4.42 (s, 1H), 4.03-4.00 (m, 1H), 3.63-3.60 (m, 1H), 2.91-2.86 (m, 1H), 2.78-2.73 (m, 1H), 2.68-2.62 (m, 1H), 2.08-1.92 (m, 2H), 1.69-1.68 (m, 2H) 1.01-0.92 (m, 4H).

Molecular formula: C₂₃H₂₃N₅O₃S Precise molecular weight: 449.15 LC-MS (m/z): 450.07 [M+H]⁺.

### Example 109: Synthesis of (R)-3-hydroxy-4-(4-((1-(methylsulfonyl)piperidin-3-yl)amino)phthalazin-1-yl)benzonitrile (Compound 102) Referring to the Aforementioned Examples

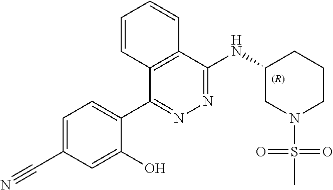

¹HNMR (400 MHz, DMSO-d6) δ (ppm): 10.46 (s, 1H), 8.43-8.41 (d, J=8 Hz, 1H), 7.89 (t, J=8 Hz, 1H), 7.82-7.81 (m, 1H), 7.52-7.50 (d, J=8 Hz, 1H), 7.43-7.42 (d, J=4 Hz, 1H), 7.33-7.32 (m, 2H), 4.43 (s, 1H), 4.01-3.98 (m, 1H), 3.56-3.53 (m, 1H), 2.90 (s, 3H), 2.80-2.75 (m, 1H), 2.65-2.60 (m, 1H), 2.06-1.93 (m, 2H), 1.71-1.69 (m, 2H).

Molecular formula: C₂₁H₂₁N₅O₃S Precise molecular weight: 423.14 LC-MS (m/z): 424.06 [M+H]⁺.

### Example 110: Synthesis of (R)—N-(4-(4-cyano-2-hydroxyphenyl)phthalazin-1-yl)-1-methylpiperidine-3-carboxamide (Compound 113) Referring to the Aforementioned Examples

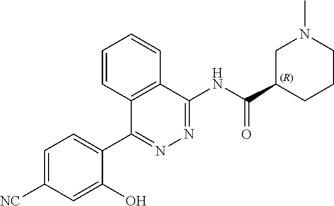

¹HNMR (400 MHz, DMSO-d6) δ (ppm): 11.17 (s, 1H), 10.76 (s, 1H), 8.03-7.97 (m, 3H), 7.64-7.57 (m, 2H), 7.49-7.44 (m, 2H), 3.55 (s, 1H), 3.22 (s, 2H), 3.11-3.09 (m, 1H), 2.89 (s, 1H), 2.75 (s, 3H), 2.24 (s, 1H), 2.01-1.65 (m, 4H).

Molecular formula: C₂₂H₂₁N₅O₂ Precise molecular weight: 387.17 LC-MS (m/z): 388.09 [M+H]⁺.

### Example 111: Synthesis of 3-hydroxy-4-(4-(((cis)-3-hydroxycyclobutyl)amino)phthalazin-1-yl)benzonitrile (Compound 117) Referring to the Aforementioned Examples

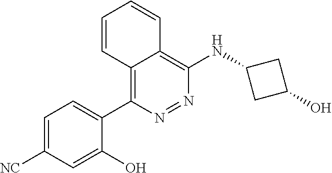 [SMILES: N#Cc1ccc(-c2nnc(N[C@H]3C[C@@H](O)C3)c3ccccc23)c(O)c1]

¹HNMR (400 MHz, DMSO-d6) δ (ppm): 11.07 (s, 1H), 10.15 (s, 1H), 9.00-8.98 (d, J=8 Hz, 1H), 8.16-8.06 (m, 2H), 7.61-7.55 (m, 2H), 7.50-7.47 (m, 2H), 5.37 (s, 1H), 4.09-3.90 (m, 2H), 2.90-2.88 (m, 2H), 2.27-2.20 (m, 2H).

Molecular formula: C₁₉H₁₆N₄O₂ Precise molecular weight: 332.13 LC-MS (m/z): 333.02 [M+H]⁺.

### Example 112: Synthesis of 2-(4-(((R)-1-methylpiperidin-3-yl)amino)phthalazin-1-yl)-5-(methylsulfinyl)phenol (Compound 120) Referring to the Aforementioned Examples

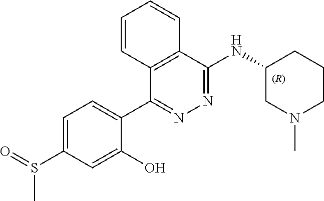

¹HNMR (400 MHz, DMSO-d6) δ (ppm): 11.06 (s, 1H), 10.33 (s, 1H), 7.81-7.73 (m, 3H), 7.54-7.52 (d, J=8 Hz, 1H), 7.25-7.23 (d, J=8 Hz, 1H), 6.87-6.83 (m, 2H), 4.70 (s, 1H), 3.69-3.66 (m, 1H), 3.46-3.40 (m, 1H), 3.14 (s, 3H), 2.43-2.33 (m, 1H), 2.13-1.97 (m, 1H), 1.61-1.55 (m, 2H).

Molecular formula: C₂₁H₂₄N₄O₂S Precise molecular weight: 396.16 LC-MS (m/z): 397.08 [M+H]⁺.

### Example 113: Synthesis of (R)-4-(4-((5,5-difluoro-1-methylpiperidin-3-yl)amino)phthalazin-1-yl)-3-hydroxybenzonitrile (Compound 121) Referring to the Aforementioned Examples

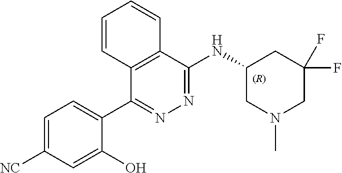

¹HNMR (400 MHz, DMSO-d6) δ (ppm): 8.34-8.32 (d, J=8 Hz, 1H), 7.92-7.88 (m, 1H), 7.83-7.79 (m, 1H), 7.50-7.39 (m, 3H), 7.34-7.30 (m, 2H), 4.71-4.61 (m, 1H), 3.18-3.03 (m, 2H), 2.49 (s, 1H), 2.51 (s, 1H), 2.40-2.29 (m, 4H), 2.10-2.05 (m, 2H).

Molecular formula: C₂₁H₁₉F₂N₅O₂ Precise molecular weight: 395.16 LC-MS (m/z): 396.07 [M+H]⁺.

### Example 114: Synthesis of 4-(4-(((3R,5S)-5-fluoro-1-methylpiperidin-3-yl)amino)phthalazin-1-yl)-3-hydroxybenzonitrile (Compound 122) Referring to the Aforementioned Examples

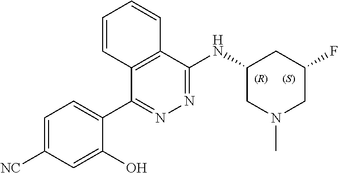

¹HNMR (400 MHz, DMSO-d6) δ (ppm): 10.47 (s, 1H), 8.36-8.34 (d, J=8 Hz, 1H), 7.89 (t, J=8 Hz, 1H), 7.80 (t, J=8 Hz, 1H), 7.50-7.49 (m, 1H), 7.43-7.41 (m, 2H), 7.33-7.28 (m, 2H), 4.86-4.48 (m, 2H), 3.09-3.07 (m, 2H), 2.44-2.43 (m, 1H), 2.29 (s, 3H), 2.06-1.90 (m, 2H), 1.77-1.66 (m, 2H).

Molecular formula: C₂₁H₂₀FN₅O Precise molecular weight: 377.17 LC-MS (m/z): 378.08 [M+H]⁺.

### Example 115: Synthesis of 3-hydroxy-4-(4-(((3R,5S)-5-hydroxy-1-methylpiperidin-3-yl)amino)phthalazin-1-yl)benzonitrile (Compound 133) Referring to the Aforementioned Examples

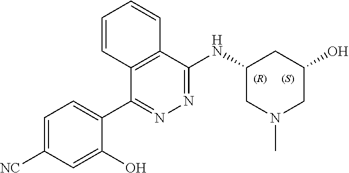

¹HNMR (400 MHz, DMSO-d6) δ (ppm): 8.39-8.37 (d, J=8 Hz, 1H), 7.87 (t, J=8 Hz, 1H), 7.79 (t, J=8 Hz, 1H), 7.50-7.48 (d, J=8 Hz, 1H), 7.43-7.41 (m, 2H), 7.34-7.28 (m, 2H), 4.95 (s, 1H), 4.47-4.46 (m, 1H), 3.72-3.67 (m, 1H), 3.17-3.09 (m, 1H), 2.90-2.89 (m, 1H), 2.20-2.27 (m, 4H), 1.91-1.80 (m, 2H), 1.47-1.34 (m, 2H).

Molecular formula: C₂₁H₂₁N₅O₂ Precise molecular weight: 375.17 LC-MS (m/z): 376.10 [M+H]⁺.

### Example 116: Synthesis of (R)-3-hydroxy-4-(4-((1-methylpiperidin-3-yl)amino)-6,7-dihydro-5H-cyclopenta[d]pyridazin-1-yl)benzonitrile (Compound 4a) Referring to the Aforementioned Examples

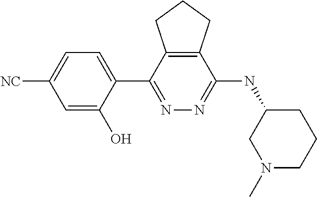 [SMILES: CN1CCC[C@@H](Nc2nnc(-c3ccc(C#N)cc3O)c3c2CCC3)C1]

¹HNMR (400 MHz, DMSO-d6) δ (ppm): 13.47 (s, 1H), 7.71-7.69 (d, J=8.1 Hz, 1H), 7.35-7.32 (m, 2H), 6.62 (s, 1H), 4.37 (s, 1H), 3.30 (s, 1H), 3.19-3.17 (m, 1H), 3.11-3.07 (m, 2H), 2.90 (s, 1H), 2.82-2.79 (m, 2H), 2.43 (s, 3H), 2.25 (br, 1H), 2.11-2.03 (m, 2H), 1.91 (m, 1H), 1.81 (s, 1H), 1.68-1.64 (m, 1H), 1.50 (s, 1H).

Molecular formula: C₂₀H₂₃N₅O Precise molecular weight: 349.19 LC-MS (m/z): 350.09 [M+H]⁺.

### Example 117: Synthesis of 3-hydroxy-4-(4-(((R)-1-methylpiperidin-3-yl)amino)-5,6,7,8-tetrahydro-5,8-methylphthalazin-1-yl)benzonitrile (Compound 16a) Referring to the Aforementioned Examples

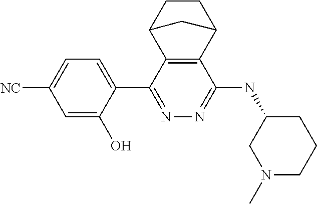 [SMILES: CN1CCC[C@@H](Nc2nnc(-c3ccc(C#N)cc3O)c3c2C2CCC3C2)C1]

¹HNMR (400 MHz, DMSO-d6) δ (ppm): 12.91 (s, 1H), 7.70-7.68 (d, J=8.4 Hz, 1H), 7.38-7.36 (m, 2H), 6.96 (s, 1H), 4.44 (s, 1H), 4.11 (s, 1H), 3.78 (s, 1H), 3.60 (s, 1H), 3.17-3.10 (m, 3H), 2.62 (s, 4H), 2.02-1.91 (m, 4H), 1.74 (s, 1H), 1.64-1.60 (m, 1H), 1.52-1.50 (m, 1H), 1.23-1.20 (m, 1H), 1.10-1.00 (m, 1H).

Molecular formula: C₂₂H₂₅N₅O Precise molecular weight: 375.21 LC-MS (m/z): 376.22 [M+H]⁺.

### Experimental Example 1: Assay on Inhibitory Activities of Compounds of the Present Invention Against NLRP3 Inflammasomes in Cells

Test Samples: The compounds of the present invention prepared according to exemplary methods.

### THP-1 is Immortalized Human Macrophage Cell Line

Test instrument: microplate reader (made from PE)

### Test Method:

1. THP-1 cells were cultured in a 1640 complete medium (500 mL 1640+56 mL FBS+560 μL 1000×P/S+2 μL mercaptoethanol) for 3-20 passages of cells.
2. Culture plate coating: 100 μL polylysine solution was added into a 96-well cell culture plate, and the reaction was carried out at 37° C. for 30 min. The solution was discarded, and PBS was washed twice for later use.
3. Induced differentiation of THP-1: THP-1 cells were resuspended in an appropriate amount of complete medium containing 10 ng/mL PMA to enable the cell suspension density to be 5×10⁵ cell/mL, then the cell suspension was added into a 96-well plate at 100 μL/well, and the plate was placed in a carbon dioxide cell incubator at 37° C. and cultured for 16 h overnight.
4. Stimulation of THP-1:
a. A serum-free THP-1 culture medium containing LPS was added, and the plate was placed in a carbon dioxide cell incubator at 37° C. and cultured for 3 h, wherein the final concentration of the culture medium was 500 ng/mL.
b. The test compounds were prepared into a mother liquor having gradient concentrations by using DMSO, and then the mother liquor was added into the cells. The resulting mixture was uniformly mixed, and finally diluted by 1:1000 times, and the cells were placed in a carbon dioxide cell incubator at 37° C. and cultured for 1 h.
c. Nigericin was added to each well at a final concentration of 10 μg/mL, and the plate was placed in a carbon dioxide cell incubator at 37° C. and cultured for 30 min.
d. The media in wells were transferred to a new culture plate, and then the plate was centrifuged at 3000 rpm for 5 min. Then the supernatant was transferred to a new 96-well plate.
e. The collected cell culture supernatant samples were tested for the content of human IL-1β by using a commercial Elisa kit, as specified in the instructions.

The test results are shown in Table 2 below.

TABLE 2
Inhibitory activities of the compounds of the
present invention against NLRP3 in THP-1 cells

| Test samples | IC50 (nM) |
|---|---|
| Compound 1 | 1.8 |
| Compound 3 | 24 |
| Compound 4 | 9 |
| Compound 5 | 14 |
| Compound 6 | 11 |
| Compound 8 | 3 |
| Compound 9 | 3 |
| Compound 12 | 4 |
| Compound 16 | 7 |
| Compound 20 | 12 |
| Compound 24 | 14 |
| Compound 32 | 15 |
| Compound 34 | 8 |
| Compound 36 | 11 |
| Compound 38 | 12 |
| Compound 42 | 13 |
| Compound 48 | 2 |
| Compound 49 | 9 |
| Compound 50 | 1 |
| Compound 54 | 13 |
| Compound 55 | 4 |
| Compound 60 | 13 |
| Compound 70 | 1 |
| Compound 72 | 1 |
| Compound 73 | 1 |
| Compound 74 | 6 |
| Compound 77 | 2 |
| Compound 78 | 2 |
| Compound 79 | 13 |
| Compound 87 | 2 |
| Compound 88 | 1 |
| Compound 89 | 7 |
| Compound 90 | 7 |
| Compound 93 | 6 |
| Compound 95 | 1 |
| Compound 101 | 5 |
| Compound 108 | 15 |
| Compound 111 | 4 |
| Compound 112 | 16 |
| Compound 116 | 21 |
| Compound 118 | 23 |
| Compound 123 | 6 |
| Compound 124 | 5 |
| Compound 125 | 3 |
| Compound 126 | 5 |
| Compound 127 | 4 |
| Compound 129 | 4 |
| Compound 1a | 7 |
| Compound 2a | 3 |
| Compound 3a | 4 |
| Compound 5a | 5 |
| Compound 6a | 1 |
| Compound 8a | 4 |
| Compound 15a | 2 |
| Compound 21a | 14 |
| Compound 23a | 6 |
| Compound 24a | 1 |
| Compound 26a | 4 |
| Compound 29a | 1 |
| Compound 31a | 6 |
| Compound 32a | 7 |

As can be seen from the experimental results in Table 2, the compounds of the present invention have good inhibitory activities against NLRP3 inflammasomes, and thus the compounds of the present invention can be used for preventing and/or treating NLRP3 inflammasome-associated diseases.

The above description is only for the purpose of illustrating preferred examples of the present invention, and is not intended to limit the scope of the present invention. Any modifications, equivalents, improvements, and the like made without departing from the spirit and principle of the present invention should be included in the protection scope of the present invention.

1. A compound of general formula (I), or a pharmaceutically acceptable salt, a stereoisomer or a tautomer thereof: 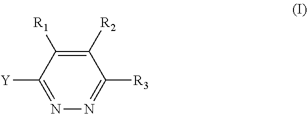
wherein,
R₁ is selected from hydrogen, hydroxyl, amino, carboxyl, cyano, nitro, halogen, C₁₋₆ alkyl, haloC₁₋₆ alkyl, C₁₋₆ alkoxy, haloC₁₋₆ alkoxy, C₂₋₆ alkenyl, C₂₋₆ alkynyl, 3-7 membered heterocyclyl, 3-7 membered cycloalkyl, aryl, and 5-7 membered heteroaryl;
R₂ is selected from hydrogen, hydroxyl, amino, carboxyl, cyano, nitro, halogen, C₁₋₆ alkyl, haloC₁₋₆ alkyl, C₁₋₆ alkoxy, haloC₁₋₆ alkoxy, C₂₋₆ alkenyl, C₂₋₆ alkynyl, 3-7 membered heterocyclyl, 3-7 membered cycloalkyl, aryl, and 5-7 membered heteroaryl;
R₁ and R₂ are respectively optionally substituted with 1-3 substituents selected from hydroxyl, amino, carboxyl, cyano, nitro, halogen, carbonyl, C₁₋₆ alkyl, haloC₁₋₆ alkyl, C₁₋₆ alkoxy, haloC₁₋₆ alkoxy, 3-7 membered heterocyclyl, 3-7 membered cycloalkyl, aryl, and 5-7 membered heteroaryl;
or,
R₁ and R₂, together with the carbon atom to which they are attached, form a 5-12 membered ring A, wherein the 5-12 membered ring A is optionally substituted with 1-4 substituents selected from hydroxyl, amino, carboxyl, cyano, nitro, halogen, carbonyl, C₁₋₆ alkyl, —NH—C₁₋₆ alkyl, haloC₁₋₆ alkyl, C₁₋₆ alkoxy, haloC₁₋₆ alkoxy, 3-7 membered heterocyclyl, 3-7 membered cycloalkyl, aryl, 5-7 membered heteroaryl, C₁₋₆ alkylsulfonyl, and —N(C₁₋₆ alkyl)₂;
R₃ is selected from —(C₁₋₆ alkylene)₀₋₂-NR₄R₅, —(C₁₋₆ alkylene)₀₋₂-NR₄—COR₅, —(C₁₋₆ alkylene)₀₋₂-CO—NR₄-R₅, and —(C₁₋₆ alkylene)₀₋₂-O—R₅; R₄ is selected from hydrogen and C₁₋₆ alkyl; R₅ is selected from 3-7 membered heterocyclyl, 3-7 membered cycloalkyl, aryl, and 5-7 membered heteroaryl; R₅ is optionally substituted with 1-4 substituents selected from halogen, cyano, amino, hydroxyl, C₁₋₆ alkyl, haloC₁₋₆ alkyl, C₁₋₆ alkoxy, 3-7 membered heterocyclyl, 3-7 membered cycloalkyl, aryl, 5-7 membered heteroaryl, C₂₋₆ alkenylcarbonyl, sulfonyl, C₁₋₆ alkylcarbonyl, and carboxyl;
Y is selected from aryl, 5-14 membered heteroaryl, 3-14 membered heterocyclyl, and 3-12 membered cycloalkyl, and Y is optionally substituted with 1-3 substituents selected from halogen, cyano, amino, hydroxyl, carbonyl, C₁₋₆ alkyl, haloC₁₋₆ alkyl, C₁₋₆ alkoxy, 3-7 membered heterocyclyl, 3-7 membered cycloalkyl, aryl, 5-7 membered heteroaryl, C₁₋₆ alkylamino, C₁₋₆ alkylcarbonylamino, C₁₋₆ alkylsulfonyl, aminocarbonyl, C₁₋₆ alkylaminocarbonyl, sulfonyl, C₁₋₆ alkylthio, and C₁₋₆ alkylsulfinyl;
when R₅ is substituted,
the substituents on R₅, which are C₁₋₆ alkyl, haloC₁₋₆ alkyl, C₁₋₆ alkoxy, 3-7 membered heterocyclyl, 3-7 membered cycloalkyl, aryl, 5-7 membered heteroaryl, and sulfonyl, are optionally substituted with 1-3 substituents selected from halogen, cyano, amino, hydroxyl, carbonyl, C₁₋₆ alkyl, and 3-6 membered cycloalkyl;
when Y is substituted,
the substituents on Y, which are C₁₋₆ alkyl, haloC₁₋₆ alkyl, C₁₋₆ alkoxy, 3-7 membered heterocyclyl, 3-7 membered cycloalkyl, aryl, 5-7 membered heteroaryl, and sulfonyl, are optionally substituted with 1-3 substituents selected from halogen, cyano, amino, hydroxyl, carbonyl, C₁₋₆ alkyl, and 3-6 membered cycloalkyl.

2. The compound, or the pharmaceutically acceptable salt, the stereoisomer or the tautomer thereof according to claim 1, having a structure of general formula (II): 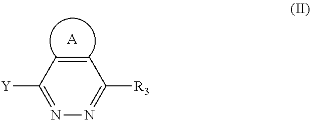
wherein ring A is selected from 5-7 membered cycloalkenyl, 5-7 membered cycloalkyl, 5-7 membered heterocyclyl, phenyl, and 5-7 membered heteroaryl; ring A is optionally substituted with 1-4 substituents selected from hydroxyl, amino, carboxyl, cyano, nitro, halogen, C₁₋₆ alkyl, —NH—C₁₋₆ alkyl, haloC₁₋₆ alkyl, C₁₋₆ alkoxy, haloC₁₋₆ alkoxy, 3-7 membered heterocyclyl, 3-7 membered cycloalkyl, aryl, 5-7 membered heteroaryl, C₁₋₆ alkylsulfonyl, and —N(C₁₋₆ alkyl)₂.

3. The compound, or the pharmaceutically acceptable salt, the stereoisomer or the tautomer thereof according to claim 2,
wherein ring A is selected from phenyl and 5-7 membered heteroaryl; ring A is optionally substituted with 1-4 substituents selected from hydroxyl, amino, carboxyl, cyano, nitro, halogen, C₁₋₆ alkyl, —NH—C₁₋₆ alkyl, haloC₁₋₆ alkyl, C₁₋₆ alkoxy, haloC₁₋₆ alkoxy, 3-7 membered heterocyclyl, 3-7 membered cycloalkyl, C₁₋₆ alkylsulfonyl, and —N(C₁₋₆ alkyl)₂; or
wherein Ring A is selected from 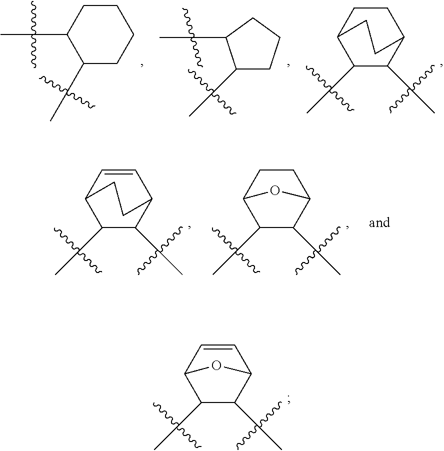 [SMILES: *C1CCCCC1* ; *C1CCCC1* ; *C1C2CCC(CC2)C1* ; *C1C2C=CC(CC2)C1* ; *C1C2CCC(O2)C1* ; *C1C2C=CC(O2)C1*]
ring A is optionally substituted with 1-4 substituents selected from hydroxyl, amino, carboxyl, cyano, nitro, halogen, C₁₋₆ alkyl, —NH—C₁₋₆ alkyl, haloC₁₋₆ alkyl, C₁₋₆ alkoxy, haloC₁₋₆ alkoxy, 3-7 membered heterocyclyl, 3-7 membered cycloalkyl, C₁₋₆ alkylsulfonyl, and —N(C₁₋₆ alkyl)₂; or
where ring A is selected from 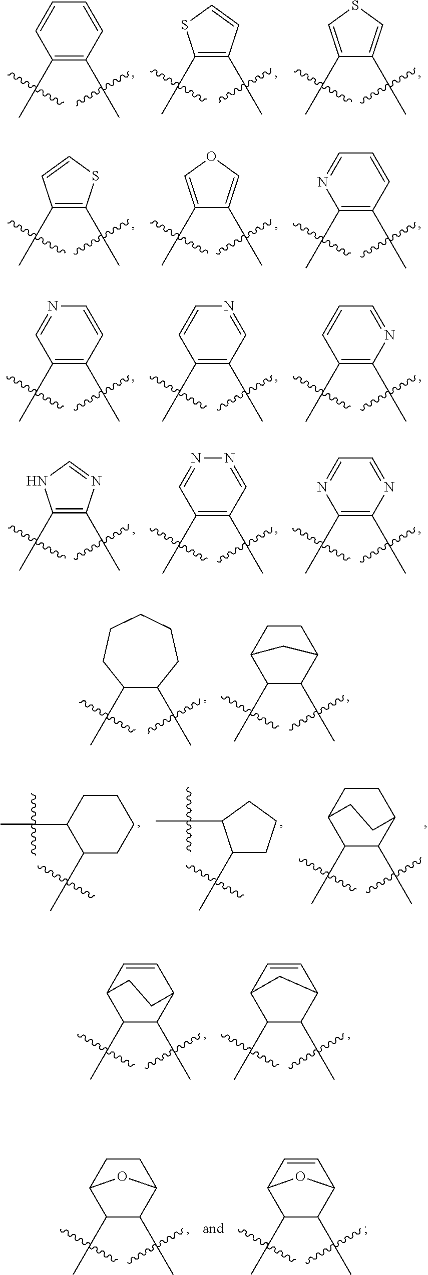
ring A is optionally substituted with 1-2 substituents selected from cyano, halogen, C₁₋₆ alkyl, —NH—C₁₋₆ alkyl, haloC₁₋₆ alkyl, C₁₋₆ alkoxy, 3-7 membered cycloalkyl, 5-7 membered heteroaryl, C₁₋₆ alkylsulfonyl, and —N(C₁₋₆ alkyl)₂.

4. (canceled)

5. The compound, or the pharmaceutically acceptable salt, the stereoisomer or the tautomer thereof according to claim 1,
wherein Y is selected from phenyl, 5-7 membered heteroaryl, 3-8 membered heterocyclyl, and 3-7 membered cycloalkyl; Y is optionally substituted with 1-3 substituents selected from halogen, cyano, amino, hydroxyl, carbonyl, C₁₋₆ alkyl, haloC₁₋₆ alkyl, C₁₋₆ alkoxy, 3-7 membered heterocyclyl, 3-7 membered cycloalkyl, C₁₋₆ alkylamino, C₁₋₆ alkylcarbonylamino, C₁₋₆ alkylsulfonyl, aminocarbonyl, C₁₋₆ alkylaminocarbonyl, and sulfonyl; the C₁₋₆ alkyl, haloC₁₋₆ alkyl, C₁₋₆ alkoxy, 3-7-membered heterocyclyl, 3-7-membered cycloalkyl and sulfonyl are optionally substituted with 1-3 substituents selected from halogen, cyano, amino, hydroxyl, carbonyl, and C₁₋₆ alkyl, or
wherein Y is selected from naphthyl, 8-14 member fused heteroaryl, 6-12 membered fused heterocyclyl, and 6-12 membered fused cycloalkyl; Y is optionally substituted with 1-3 substituents selected from halogen, cyano, amino, hydroxyl, carbonyl, C₁₋₆ alky, haloC₁₋₆ alkyl, and C₁₋₆ alkoxy; or
wherein Y is selected from 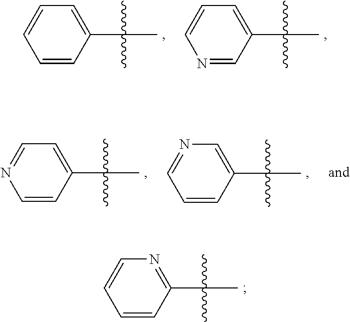 [SMILES: *c1ccccc1 ; *c1cccnc1 ; *c1ccncc1 ; *c1cccnc1 ; *c1ccccn1]
Y is sibstituted with 1-3 substituents selected from halogen, cyano, hydroxyl, amino, C₁₋₆ alkyl, C₁₋₆ alkoxy, hydroxyl-substituted C₁₋₆ alkyl, haloC₁₋₆ alkyl, aminocarbonyl, C₁₋₆ alkylamino, C₁₋₆ alkylcarbonylamino, C₁₋₆ alkylaminocarbonyl, 3-7 membered heterocyclyl, 3-7 membered cycloalkyl, 5-7 membered heteroaryl, C₁₋₆ alkylsulfonyl, C₁₋₆ alkylthio, and C₁₋₆ alkylsulfinyl.

6. (canceled)

7. The compound, or the pharmaceutically acceptable salt, the stereoisomer or the tautomer thereof according to claim 1
wherein Y is substituted with cyano and is optionally substituted with 1-2 substituents selected from halogen, cyano, amino, hydroxyl, carbonyl, C₁₋₆ alkyl, haloC₁₋₆ alkyl, C₁₋₆ alkoxy, 3-7 membered heterocyclyl, 3-7 membered cycloalkyl, C₁₋₆ alkylamino, C₁₋₆ alkylcarbonylamino, C₁₋₆ alkylsulfonyl, aminocarbonyl, C₁₋₆ alkylaminocarbonyl, and sulfonyl; the C₁₋₆ alkyl, haloC₁₋₆ alkyl, C₁₋₆ alkoxy, 3-7-membered heterocyclyl, 3-7-membered cycloalkyl and sulfonyl are optionally substituted with 1-3 substituents selected from halogen, cyano, amino, hydroxyl, carbonyl, and C₁₋₆ alkyl.

8. The compound, or the pharmaceutically acceptable salt, the stereoisomer or the tautomer thereof according to claim 1,
wherein R₃ is selected from —NR₄R₅, —NH—COR₅, and —O—R₅; R₄ is selected from hydrogen and C₁₋₃ alkyl; R₅ is selected from 3-7 membered cycloalkyl and 3-7 membered heterocyclyl, and R₅ is optionally substituted with 1-2 substituents selected from C₁₋₆ alkyl, hydroxyl, hydroxyl-substituted C₁₋₆ alkyl, 3-7 membered cycloalkyl-substituted C₁₋₆ alkyl, hydroxyl, halogen, C₂₋₆ alkenylcarbonyl, C₁₋₆ alkylsulfonyl, 3-7 membered cycloalkylsulfonyl, aminosulfonyl, 3-7 membered heterocyclyl, 3-7 membered cycloalkyl, carboxyl, and C₁₋₆ alkylcarbonyl; or
where R₃ is —NH—R₅, and R₅ is 3-7 membered heterocyclyl substituted with 1-2 substituents selected from C₁₋₆ alkyl.

9. (canceled)

10. The compound, or the pharmaceutically acceptable salt, the stereoisomer or the tautomer thereof according to claim 3,
wherein Y is selected from phenyl, 5-7 membered heteroaryl, 3-8 membered heterocyclyl, and 3-7 membered cycloalkyl; Y is optionally substituted with 1-3 substituents selected from halogen, cyano, amino, hydroxyl, carbonyl, C₁₋₆ alkyl, haloC₁₋₆ alkyl, C₁₋₆ alkoxy, 3-7 membered heterocyclyl, 3-7 membered cycloalkyl, C₁₋₆ alkylamino, C₁₋₆ alkylcarbonylamino, C₁₋₆ alkylsulfonyl, aminocarbonyl, C₁₋₆ alkylaminocarbonyl, and sulfonyl; the C₁₋₆ alkyl, haloC₁₋₆ alkyl, C₁₋₆ alkoxy, 3-7-membered heterocyclyl, 3-7-membered cycloalkyl and sulfonyl are optionally substituted with 1-3 substituents selected from halogen, cyano, amino, hydroxyl, carbonyl, and C₁₋₆ alkyl;
R₃ is —NH—R₅, and R₅ is 3-7 membered heterocyclyl substituted with 1-2 substituents selected from C₁₋₆ alkyl.

11. The compound, or the pharmaceutically acceptable salt, the stereoisomer or the tautomer thereof according to claim 1,
wherein R₁ and R₂, together with the carbon atom to which they are attached, form a 5-8 membered ring A, wherein the 5-8 membered ring A is selected from 5-8 membered cycloalkyl, 5-8 membered cycloalkenyl, 5-8 membered heterocyclyl, phenyl, and 5-8 membered heteroaryl; ring A is optionally substituted with 1-2 substituents selected from cyano, halogen, C₁₋₆ alkyl, —NH—C₁₋₆ alkyl, haloC₁₋₆ alkyl, C₁₋₆ alkoxy, 3-7 membered cycloalkyl, 5-7 membered heteroaryl, C₁₋₆ alkylsulfonyl, and —N(C₁₋₆ alkyl)₂;
Y is selected from phenyl and 5-7 membered heteroaryl; Y is substituted with 1-3 substituents selected from halogen, cyano, hydroxyl, amino, C₁₋₆ alkyl, C₁₋₆ alkoxy, hydroxyl-substituted C₁₋₆ alkyl, haloC₁₋₆ alkyl, aminocarbonyl, C₁₋₆ alkylamino, C₁₋₆ alkylcarbonylamino, C₁₋₆ alkylaminocarbonyl, 3-7 membered heterocyclyl, 3-7 membered cycloalkyl, 5-7 membered heteroaryl, C₁₋₆ alkylsulfonyl, C₁₋₆ alkylthio, and C₁₋₆ alkylsulfinyl;
R₃ is selected from —NR₄R₅, —NH—COR₅, and —O—R₅; R₄ is selected from hydrogen and C₁₋₃ alkyl; R₅ is selected from 3-7 membered cycloalkyl and 3-7 membered heterocyclyl, and R₅ is optionally substituted with 1-2 substituents selected from C₁₋₆ alkyl, hydroxyl or 3-7 membered cycloalkyl-substituted C₁₋₆ alkyl, hydroxyl, halogen, C₂₋₆ alkenylcarbonyl, C₁₋₆ alkylsulfonyl, 3-7 membered cycloalkylsulfonyl, aminosulfonyl, 3-7 membered heterocyclyl, 3-7 membered cycloalkyl, carboxyl, and C₁₋₆ alkylcarbonyl.

12. (canceled)

13. (canceled)

14. A compound of the following formula, or a pharmaceutically acceptable salt, a stereoisomer or a tautomer thereof:

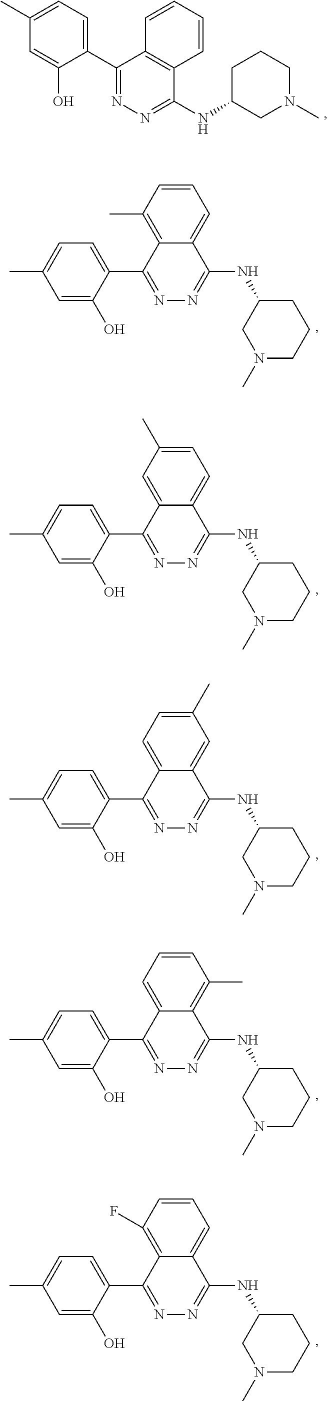

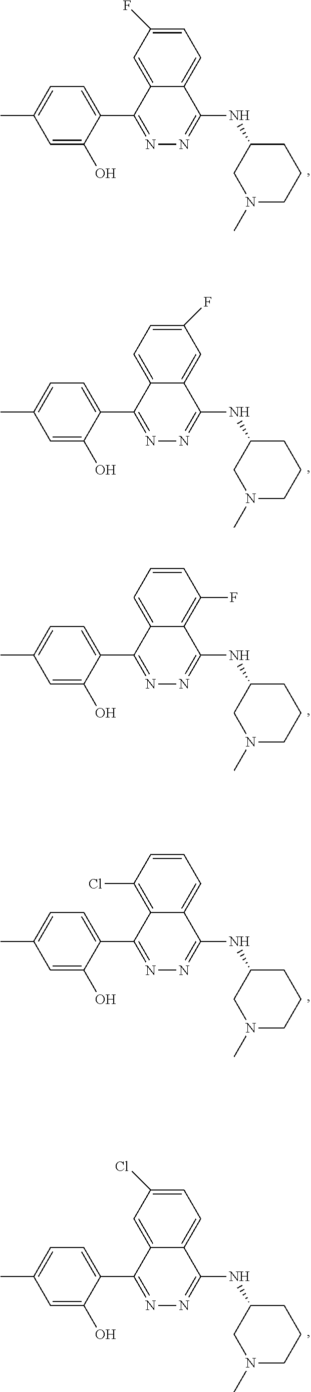 [SMILES: Cc1ccc(-c2nnc(N[C@@H]3CCCN(C)C3)c3ccc(F)cc23)c(O)c1 ; Cc1ccc(-c2nnc(N[C@@H]3CCCN(C)C3)c3cc(F)ccc23)c(O)c1 ; Cc1ccc(-c2nnc(N[C@@H]3CCCN(C)C3)c3c(F)cccc23)c(O)c1 ; Cc1ccc(-c2nnc(N[C@@H]3CCCN(C)C3)c3cccc(Cl)c23)c(O)c1 ; Cc1ccc(-c2nnc(N[C@@H]3CCCN(C)C3)c3ccc(Cl)cc23)c(O)c1]

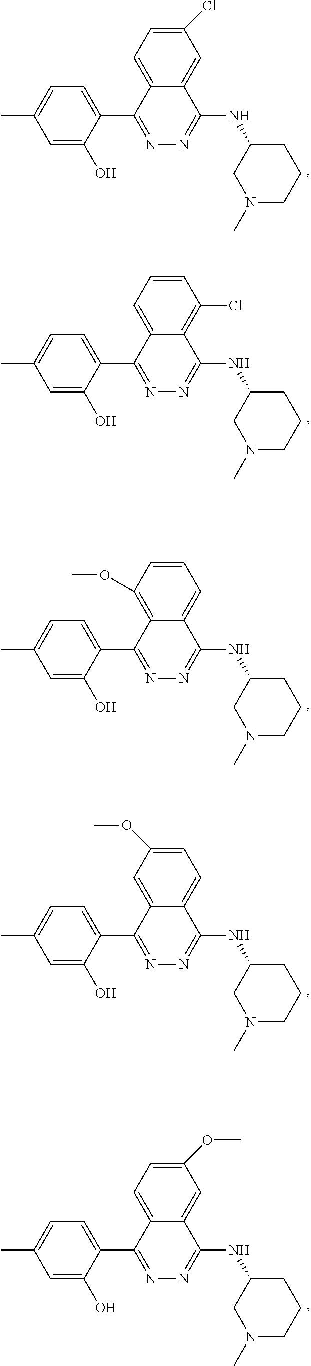 [SMILES: Cc1ccc(-c2nnc(N[C@@H]3CCCN(C)C3)c3cc(Cl)ccc23)c(O)c1 ; Cc1ccc(-c2nnc(N[C@@H]3CCCN(C)C3)c3c(Cl)cccc23)c(O)c1 ; COc1cccc2c(N[C@@H]3CCCN(C)C3)nnc(-c3ccc(C)cc3O)c12 ; COc1ccc2c(N[C@@H]3CCCN(C)C3)nnc(-c3ccc(C)cc3O)c2c1 ; COc1ccc2c(-c3ccc(C)cc3O)nnc(N[C@@H]3CCCN(C)C3)c2c1]

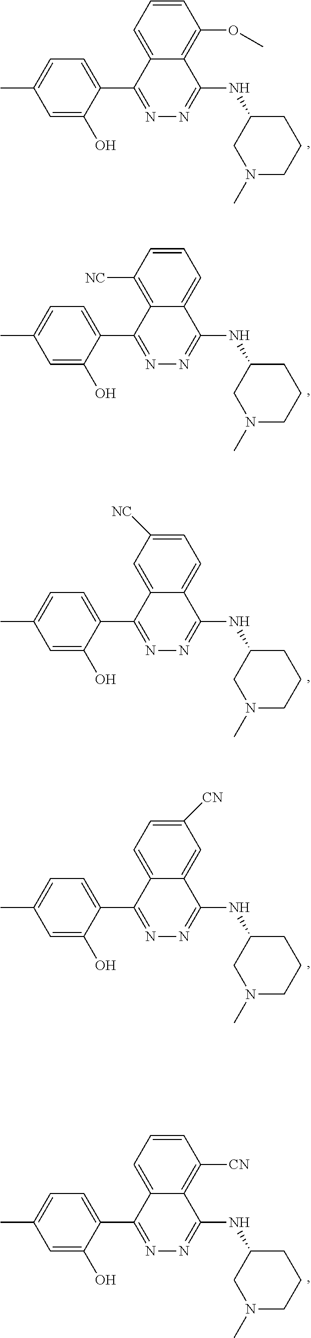 [SMILES: COc1cccc2c(-c3ccc(C)cc3O)nnc(N[C@@H]3CCCN(C)C3)c12 ; Cc1ccc(-c2nnc(N[C@@H]3CCCN(C)C3)c3cccc(C#N)c23)c(O)c1 ; Cc1ccc(-c2nnc(N[C@@H]3CCCN(C)C3)c3ccc(C#N)cc23)c(O)c1 ; Cc1ccc(-c2nnc(N[C@@H]3CCCN(C)C3)c3cc(C#N)ccc23)c(O)c1 ; Cc1ccc(-c2nnc(N[C@@H]3CCCN(C)C3)c3c(C#N)cccc23)c(O)c1]

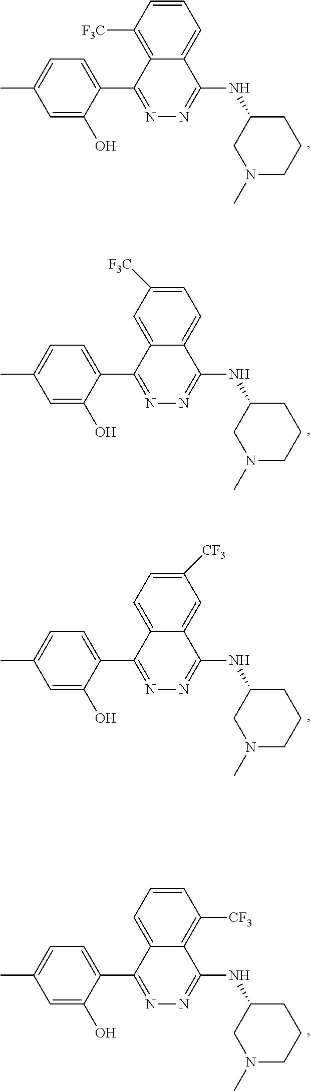 [SMILES: Cc1ccc(-c2nnc(N[C@@H]3CCCN(C)C3)c3cccc(C(F)(F)F)c23)c(O)c1 ; Cc1ccc(-c2nnc(N[C@@H]3CCCN(C)C3)c3ccc(C(F)(F)F)cc23)c(O)c1 ; Cc1ccc(-c2nnc(N[C@@H]3CCCN(C)C3)c3cc(C(F)(F)F)ccc23)c(O)c1 ; Cc1ccc(-c2nnc(N[C@@H]3CCCN(C)C3)c3c(C(F)(F)F)cccc23)c(O)c1]

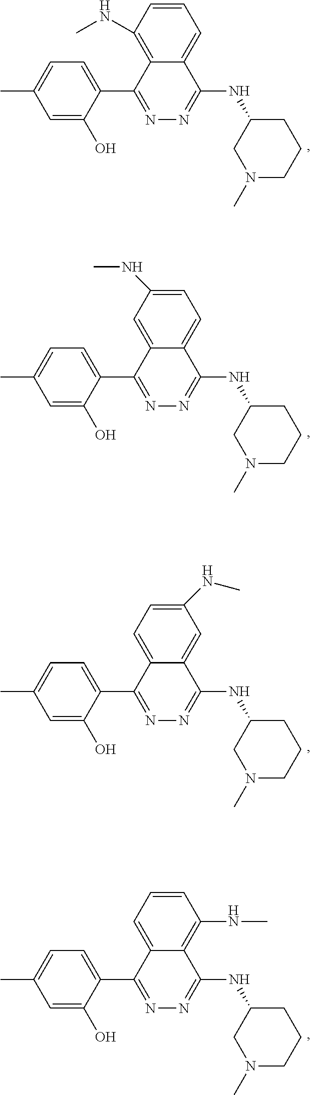 [SMILES: CNc1cccc2c(N[C@@H]3CCCN(C)C3)nnc(-c3ccc(C)cc3O)c12 ; CNc1ccc2c(N[C@@H]3CCCN(C)C3)nnc(-c3ccc(C)cc3O)c2c1 ; CNc1ccc2c(-c3ccc(C)cc3O)nnc(N[C@@H]3CCCN(C)C3)c2c1 ; CNc1cccc2c(-c3ccc(C)cc3O)nnc(N[C@@H]3CCCN(C)C3)c12]

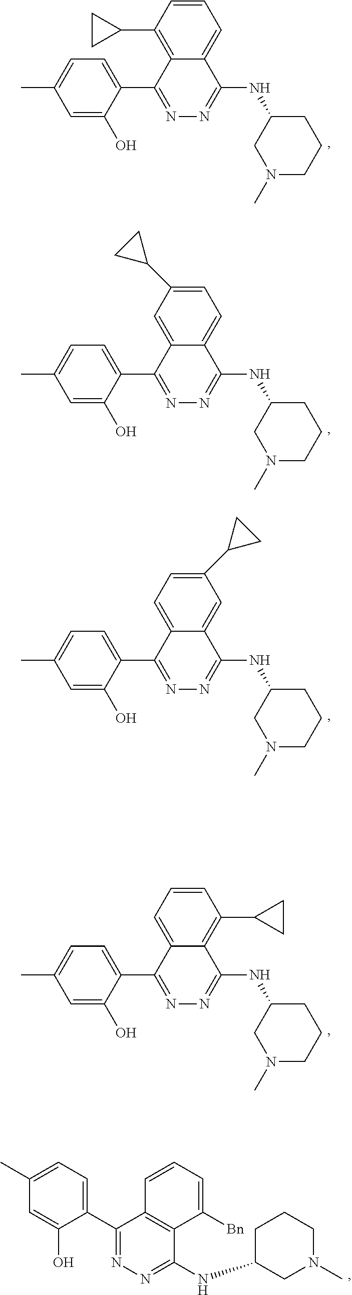

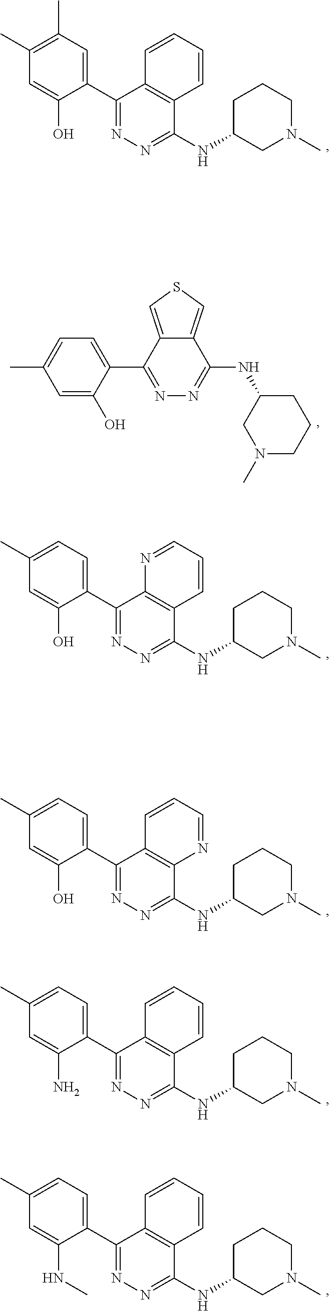

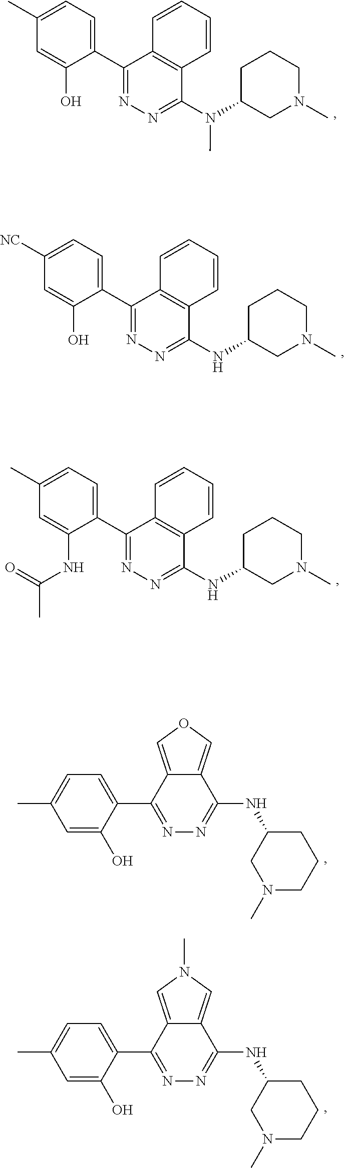

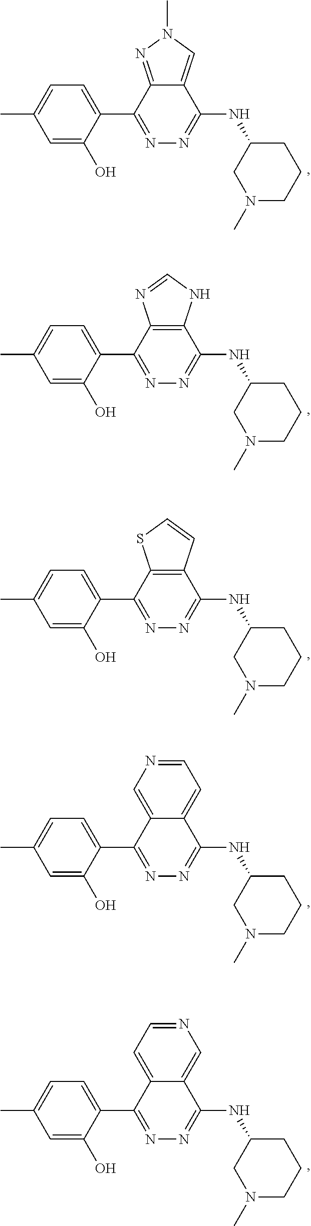 [SMILES: Cc1ccc(-c2nnc(N[C@@H]3CCCN(C)C3)c3cn(C)nc23)c(O)c1 ; Cc1ccc(-c2nnc(N[C@@H]3CCCN(C)C3)c3[nH]cnc23)c(O)c1 ; Cc1ccc(-c2nnc(N[C@@H]3CCCN(C)C3)c3ccsc23)c(O)c1 ; Cc1ccc(-c2nnc(N[C@@H]3CCCN(C)C3)c3ccncc23)c(O)c1 ; Cc1ccc(-c2nnc(N[C@@H]3CCCN(C)C3)c3cnccc23)c(O)c1]

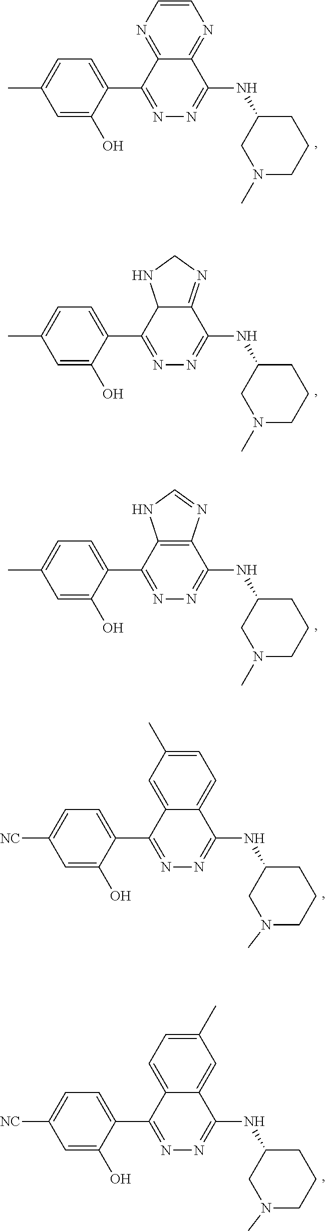 [SMILES: Cc1ccc(-c2nnc(N[C@@H]3CCCN(C)C3)c3nccnc23)c(O)c1 ; Cc1ccc(C2=NN=C(N[C@@H]3CCCN(C)C3)C3=NCNC32)c(O)c1 ; Cc1ccc(-c2nnc(N[C@@H]3CCCN(C)C3)c3nc[nH]c23)c(O)c1 ; Cc1ccc2c(N[C@@H]3CCCN(C)C3)nnc(-c3ccc(C#N)cc3O)c2c1 ; Cc1ccc2c(-c3ccc(C#N)cc3O)nnc(N[C@@H]3CCCN(C)C3)c2c1]

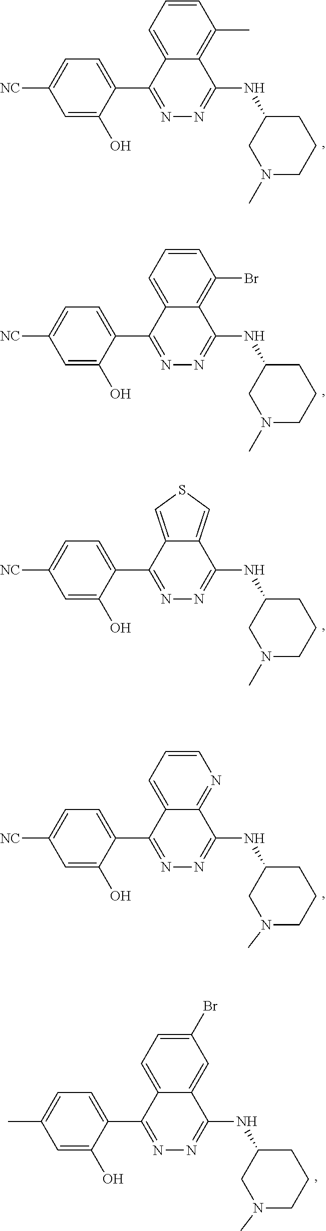 [SMILES: Cc1cccc2c(-c3ccc(C#N)cc3O)nnc(N[C@@H]3CCCN(C)C3)c12 ; CN1CCC[C@@H](Nc2nnc(-c3ccc(C#N)cc3O)c3cccc(Br)c23)C1 ; CN1CCC[C@@H](Nc2nnc(-c3ccc(C#N)cc3O)c3cscc23)C1 ; CN1CCC[C@@H](Nc2nnc(-c3ccc(C#N)cc3O)c3cccnc23)C1 ; Cc1ccc(-c2nnc(N[C@@H]3CCCN(C)C3)c3cc(Br)ccc23)c(O)c1]

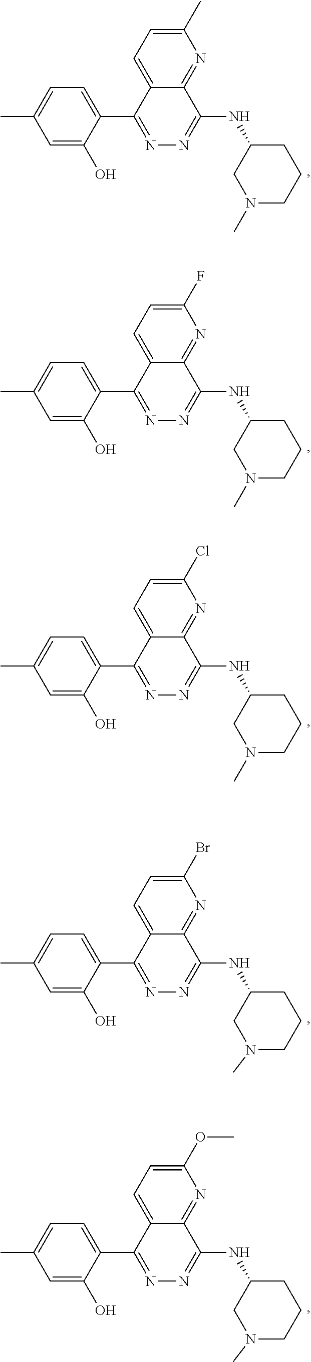 [SMILES: Cc1ccc(-c2nnc(N[C@@H]3CCCN(C)C3)c3nc(C)ccc23)c(O)c1 ; Cc1ccc(-c2nnc(N[C@@H]3CCCN(C)C3)c3nc(F)ccc23)c(O)c1 ; Cc1ccc(-c2nnc(N[C@@H]3CCCN(C)C3)c3nc(Cl)ccc23)c(O)c1 ; Cc1ccc(-c2nnc(N[C@@H]3CCCN(C)C3)c3nc(Br)ccc23)c(O)c1 ; COc1ccc2c(-c3ccc(C)cc3O)nnc(N[C@@H]3CCCN(C)C3)c2n1]

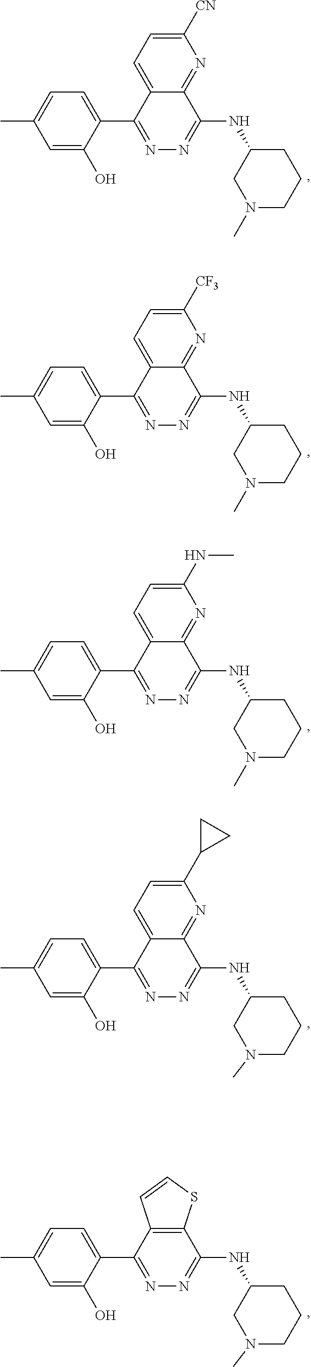

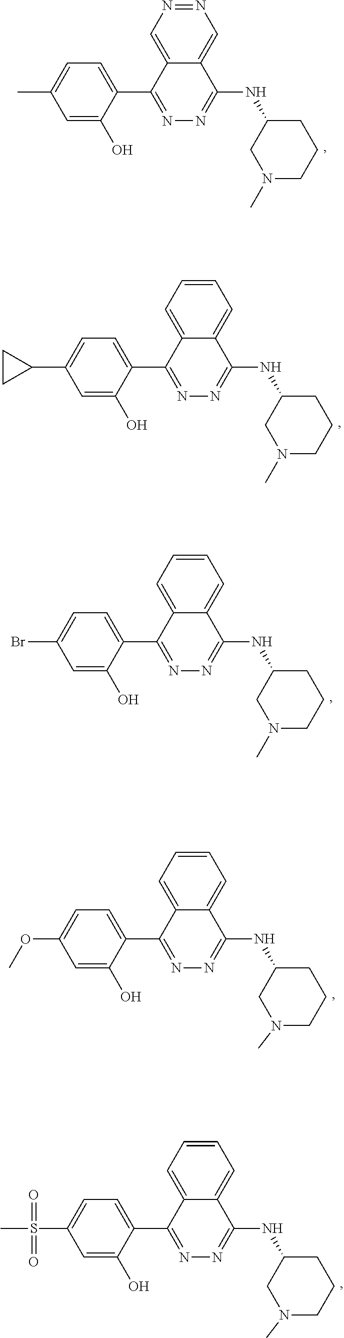 [SMILES: Cc1ccc(-c2nnc(N[C@@H]3CCCN(C)C3)c3cnncc23)c(O)c1 ; CN1CCC[C@@H](Nc2nnc(-c3ccc(C4CC4)cc3O)c3ccccc23)C1 ; CN1CCC[C@@H](Nc2nnc(-c3ccc(Br)cc3O)c3ccccc23)C1 ; COc1ccc(-c2nnc(N[C@@H]3CCCN(C)C3)c3ccccc23)c(O)c1 ; CN1CCC[C@@H](Nc2nnc(-c3ccc(S(C)(=O)=O)cc3O)c3ccccc23)C1]

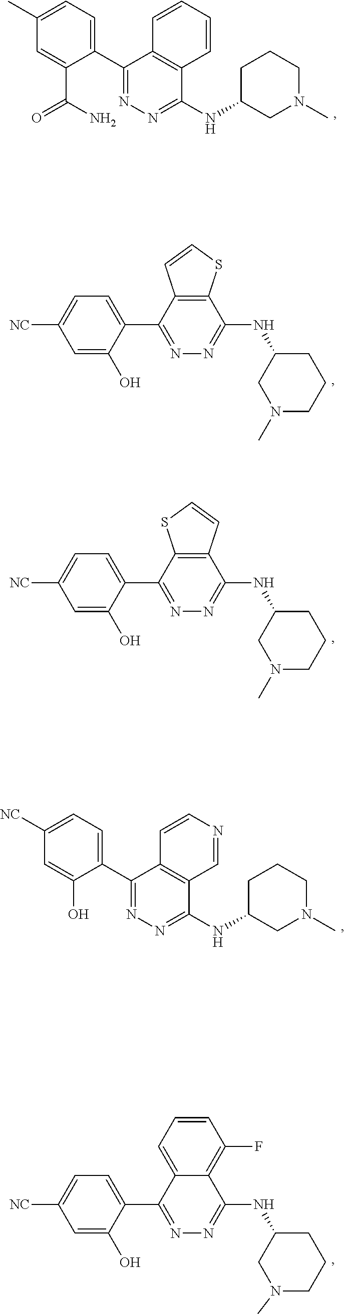

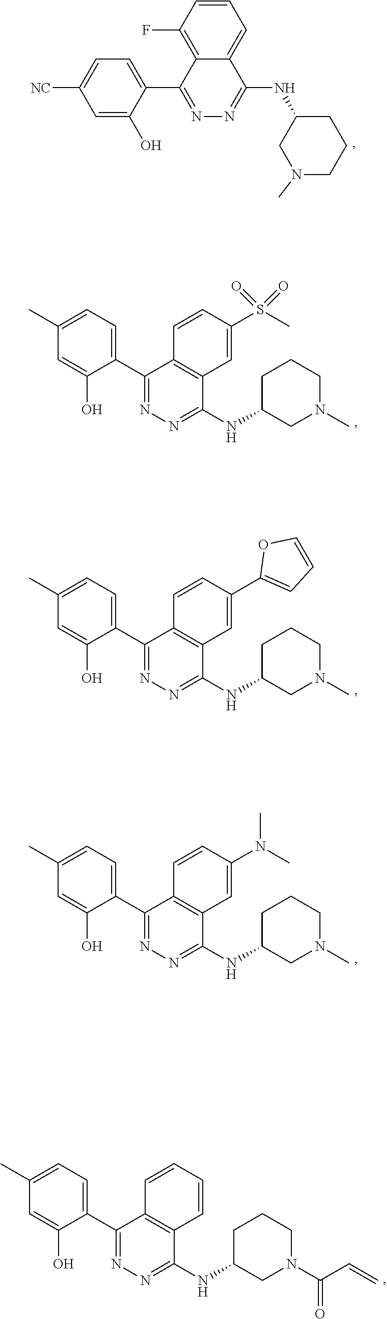

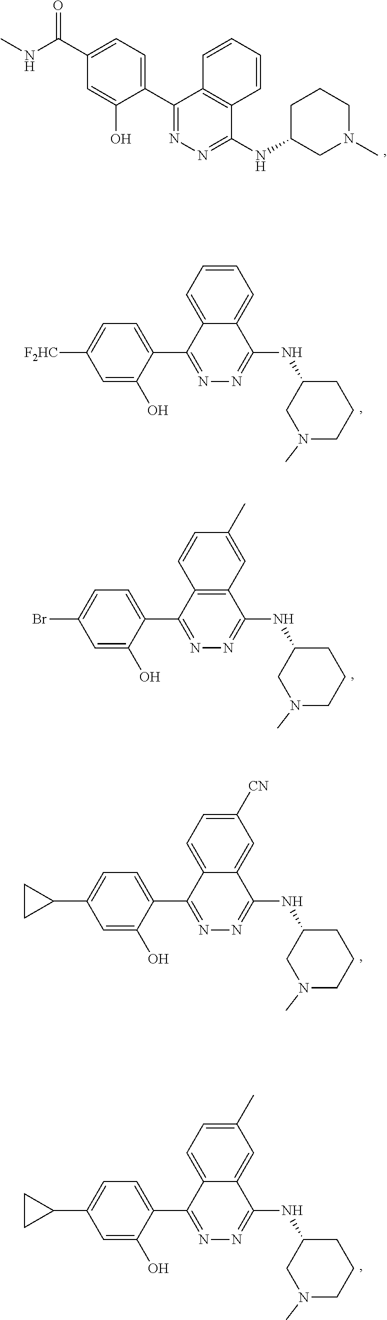

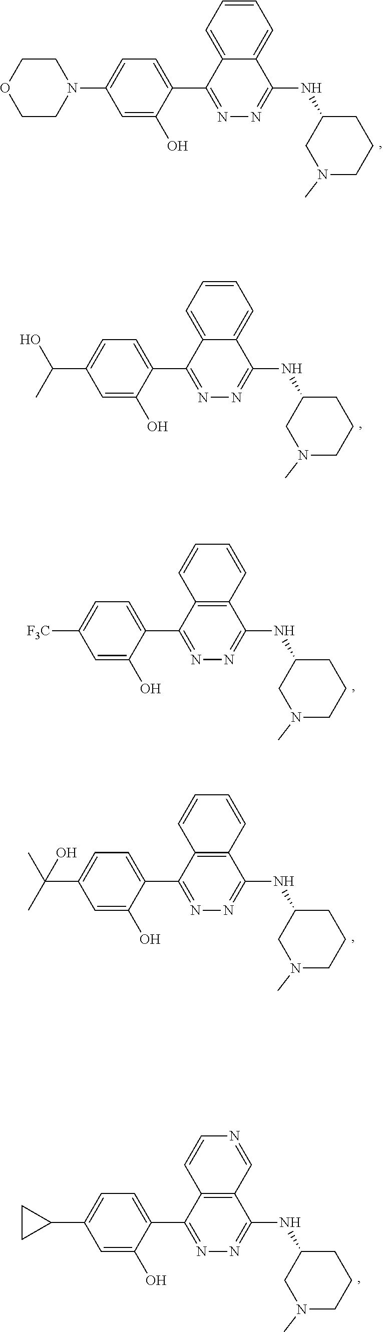 [SMILES: CN1CCC[C@@H](Nc2nnc(-c3ccc(N4CCOCC4)cc3O)c3ccccc23)C1 ; CC(O)c1ccc(-c2nnc(N[C@@H]3CCCN(C)C3)c3ccccc23)c(O)c1 ; CN1CCC[C@@H](Nc2nnc(-c3ccc(C(F)(F)F)cc3O)c3ccccc23)C1 ; CN1CCC[C@@H](Nc2nnc(-c3ccc(C(C)(C)O)cc3O)c3ccccc23)C1 ; CN1CCC[C@@H](Nc2nnc(-c3ccc(C4CC4)cc3O)c3ccncc23)C1]

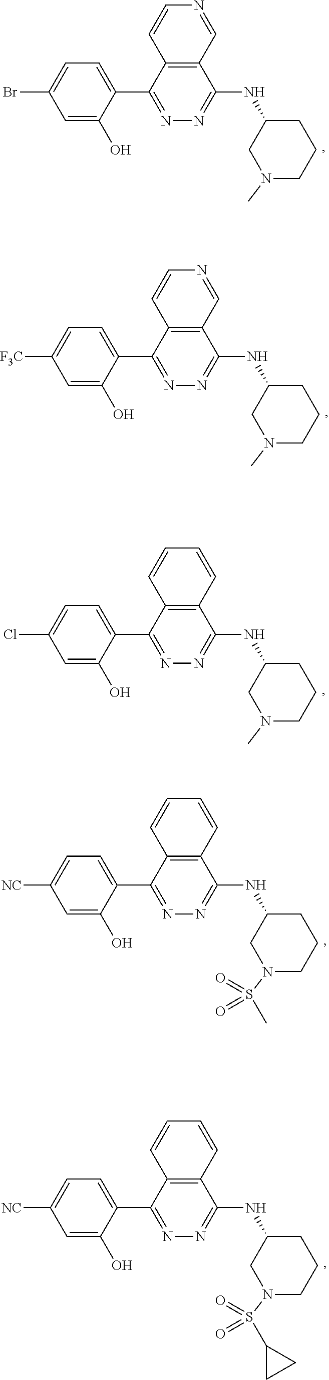 [SMILES: CN1CCC[C@@H](Nc2nnc(-c3ccc(Br)cc3O)c3ccncc23)C1 ; CN1CCC[C@@H](Nc2nnc(-c3ccc(C(F)(F)F)cc3O)c3ccncc23)C1 ; CN1CCC[C@@H](Nc2nnc(-c3ccc(Cl)cc3O)c3ccccc23)C1 ; CS(=O)(=O)N1CCC[C@@H](Nc2nnc(-c3ccc(C#N)cc3O)c3ccccc23)C1 ; N#Cc1ccc(-c2nnc(N[C@@H]3CCCN(S(=O)(=O)C4CC4)C3)c3ccccc23)c(O)c1]

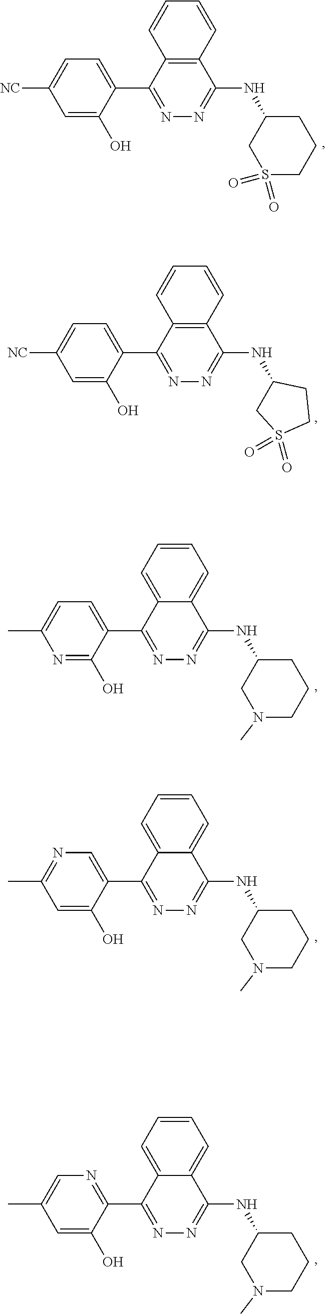 [SMILES: N#Cc1ccc(-c2nnc(N[C@@H]3CCCS(=O)(=O)C3)c3ccccc23)c(O)c1 ; N#Cc1ccc(-c2nnc(N[C@@H]3CCS(=O)(=O)C3)c3ccccc23)c(O)c1 ; Cc1ccc(-c2nnc(N[C@@H]3CCCN(C)C3)c3ccccc23)c(O)n1 ; Cc1cc(O)c(-c2nnc(N[C@@H]3CCCN(C)C3)c3ccccc23)cn1 ; Cc1cnc(-c2nnc(N[C@@H]3CCCN(C)C3)c3ccccc23)c(O)c1]

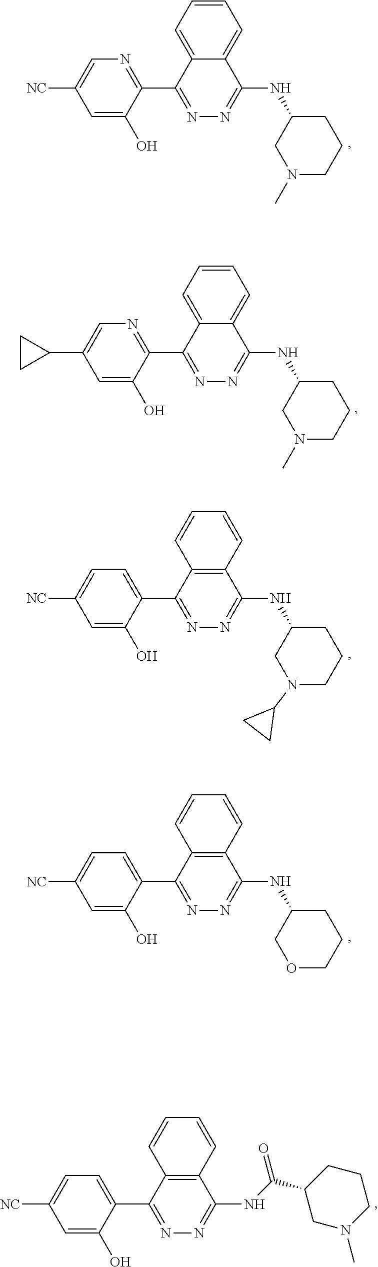 [SMILES: CN1CCC[C@@H](Nc2nnc(-c3ncc(C#N)cc3O)c3ccccc23)C1 ; CN1CCC[C@@H](Nc2nnc(-c3ncc(C4CC4)cc3O)c3ccccc23)C1 ; N#Cc1ccc(-c2nnc(N[C@@H]3CCCN(C4CC4)C3)c3ccccc23)c(O)c1 ; N#Cc1ccc(-c2nnc(N[C@@H]3CCCOC3)c3ccccc23)c(O)c1 ; CN1CCC[C@@H](C(=O)Nc2nnc(-c3ccc(C#N)cc3O)c3ccccc23)C1]

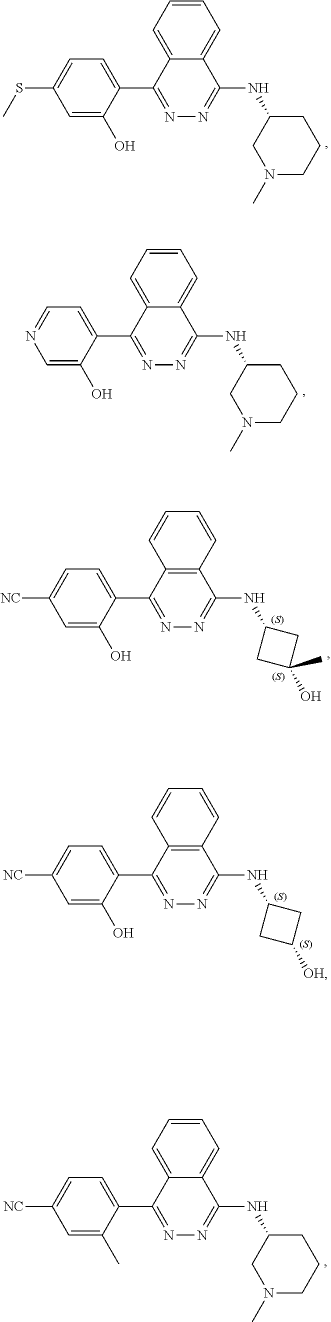

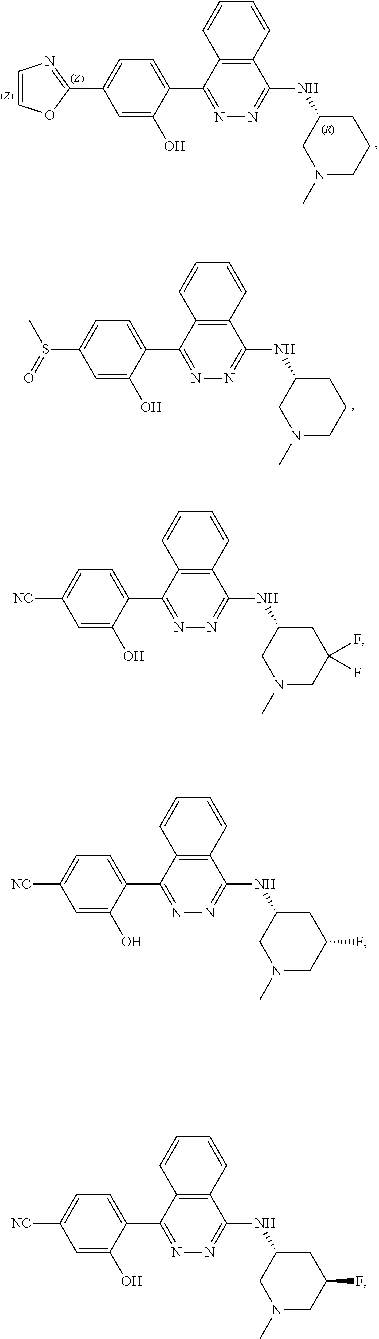

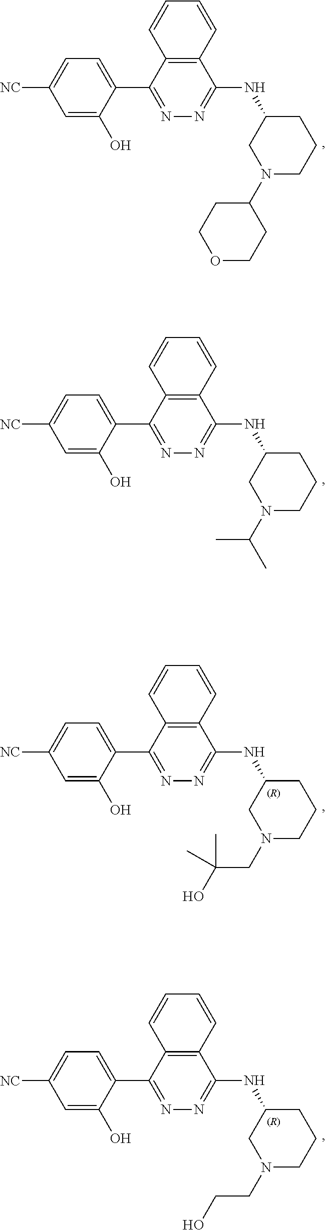

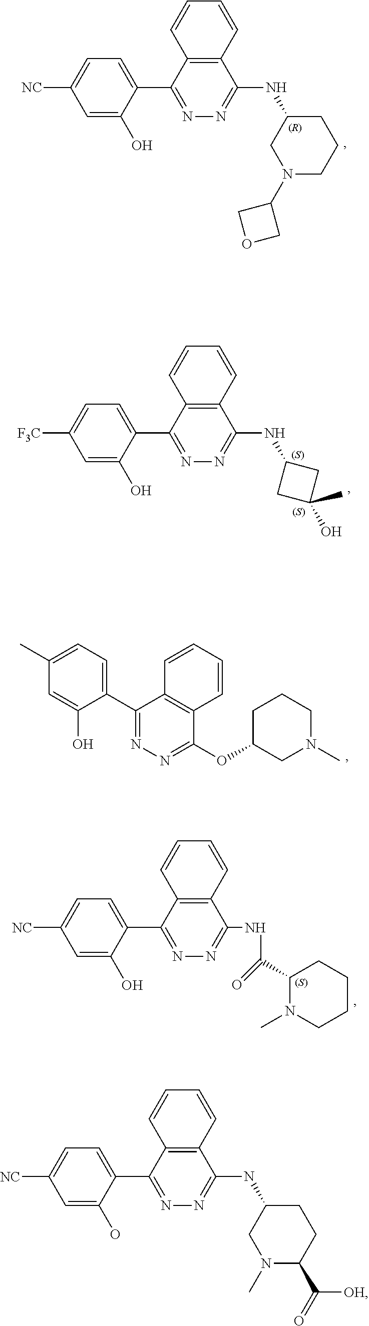

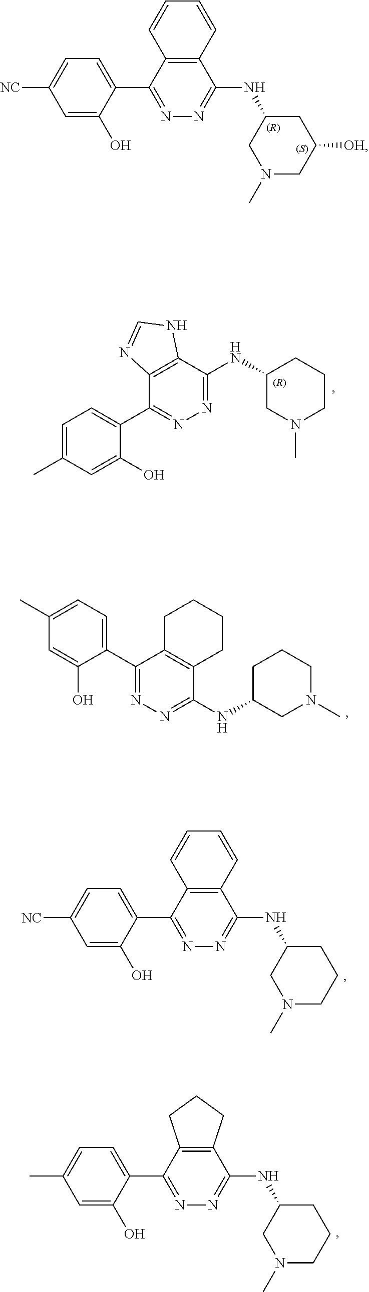

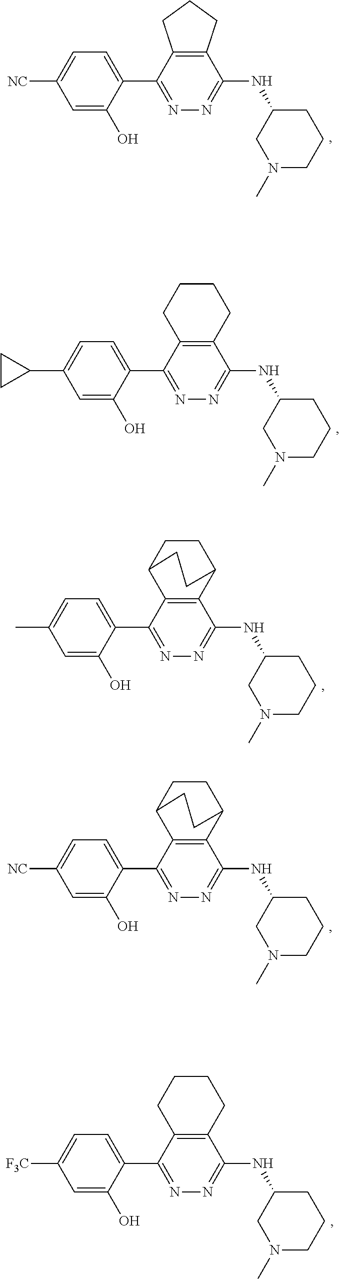 [SMILES: CN1CCC[C@@H](Nc2nnc(-c3ccc(C#N)cc3O)c3c2CCC3)C1 ; CN1CCC[C@@H](Nc2nnc(-c3ccc(C4CC4)cc3O)c3c2CCCC3)C1 ; Cc1ccc(-c2nnc(N[C@@H]3CCCN(C)C3)c3c2C2CCC3CC2)c(O)c1 ; CN1CCC[C@@H](Nc2nnc(-c3ccc(C#N)cc3O)c3c2C2CCC3CC2)C1 ; CN1CCC[C@@H](Nc2nnc(-c3ccc(C(F)(F)F)cc3O)c3c2CCCC3)C1]

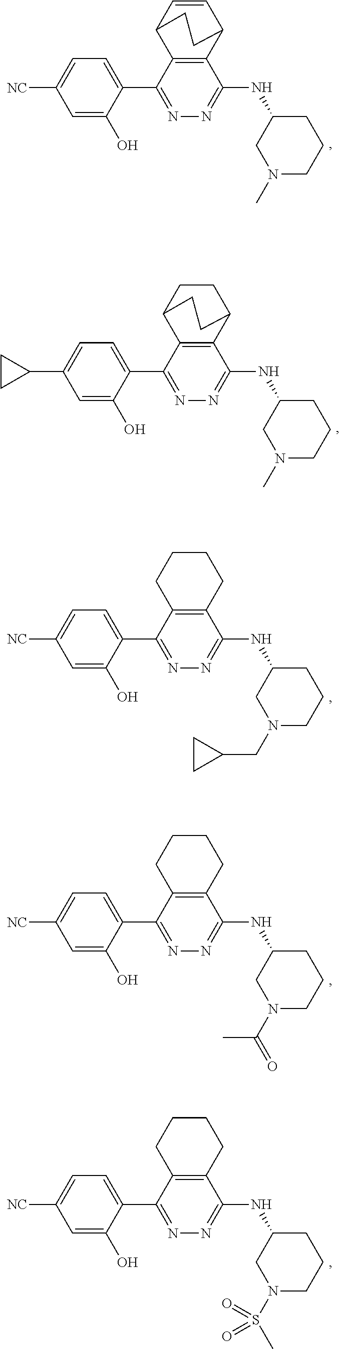 [SMILES: CN1CCC[C@@H](Nc2nnc(-c3ccc(C#N)cc3O)c3c2C2C=CC3CC2)C1 ; CN1CCC[C@@H](Nc2nnc(-c3ccc(C4CC4)cc3O)c3c2C2CCC3CC2)C1 ; N#Cc1ccc(-c2nnc(N[C@@H]3CCCN(CC4CC4)C3)c3c2CCCC3)c(O)c1 ; CC(=O)N1CCC[C@@H](Nc2nnc(-c3ccc(C#N)cc3O)c3c2CCCC3)C1 ; CS(=O)(=O)N1CCC[C@@H](Nc2nnc(-c3ccc(C#N)cc3O)c3c2CCCC3)C1]

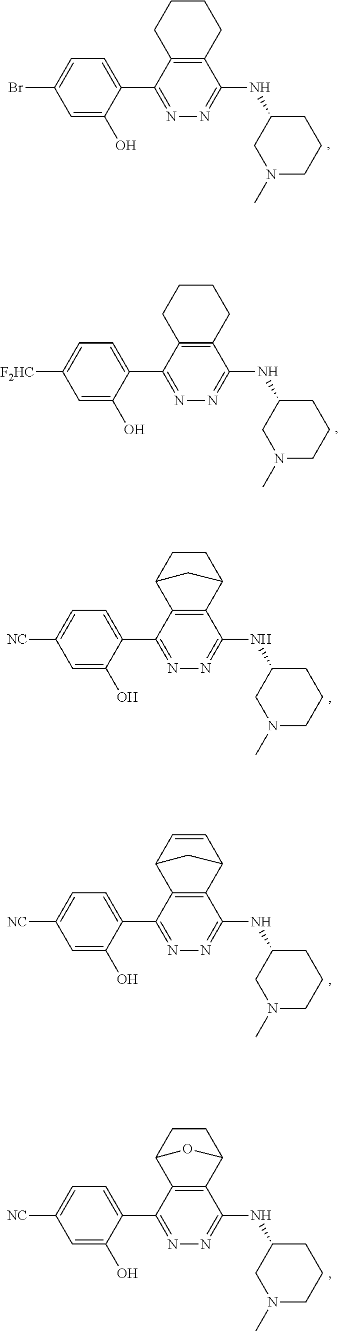 [SMILES: CN1CCC[C@@H](Nc2nnc(-c3ccc(Br)cc3O)c3c2CCCC3)C1 ; CN1CCC[C@@H](Nc2nnc(-c3ccc(C(F)F)cc3O)c3c2CCCC3)C1 ; CN1CCC[C@@H](Nc2nnc(-c3ccc(C#N)cc3O)c3c2C2CCC3C2)C1 ; CN1CCC[C@@H](Nc2nnc(-c3ccc(C#N)cc3O)c3c2C2C=CC3C2)C1 ; CN1CCC[C@@H](Nc2nnc(-c3ccc(C#N)cc3O)c3c2C2CCC3O2)C1]

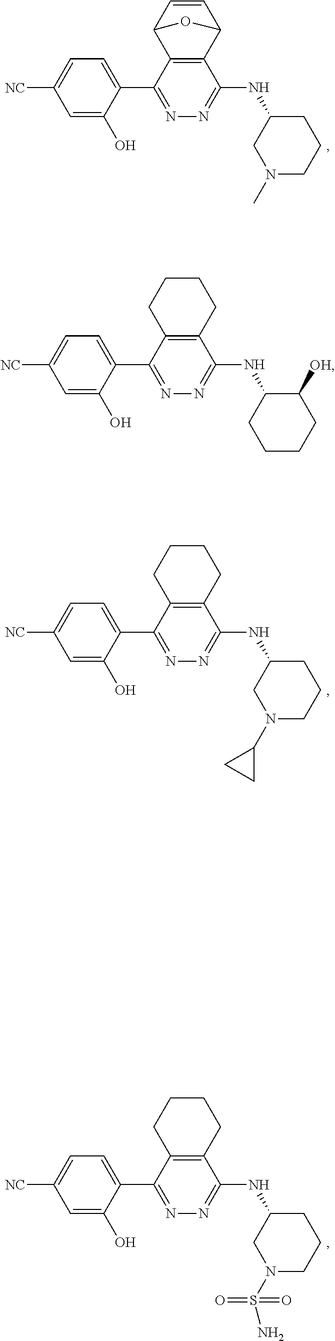 [SMILES: CN1CCC[C@@H](Nc2nnc(-c3ccc(C#N)cc3O)c3c2C2C=CC3O2)C1 ; N#Cc1ccc(-c2nnc(N[C@H]3CCCC[C@@H]3O)c3c2CCCC3)c(O)c1 ; N#Cc1ccc(-c2nnc(N[C@@H]3CCCN(C4CC4)C3)c3c2CCCC3)c(O)c1 ; N#Cc1ccc(-c2nnc(N[C@@H]3CCCN(S(N)(=O)=O)C3)c3c2CCCC3)c(O)c1]

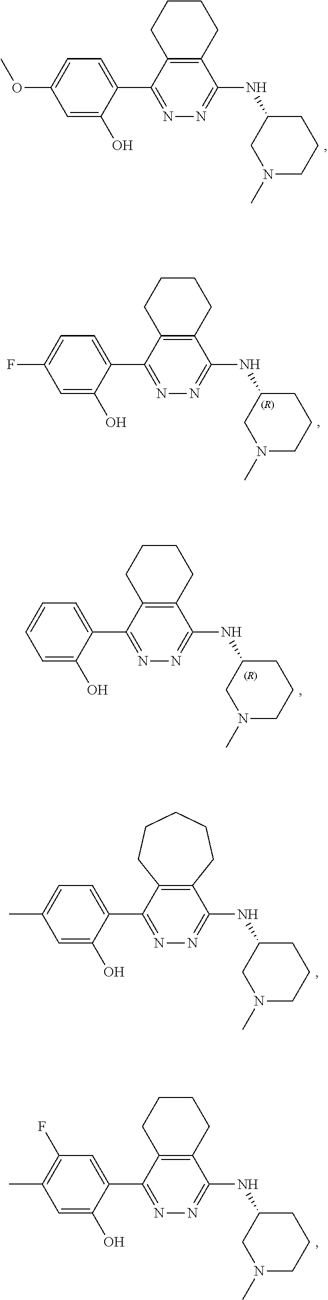

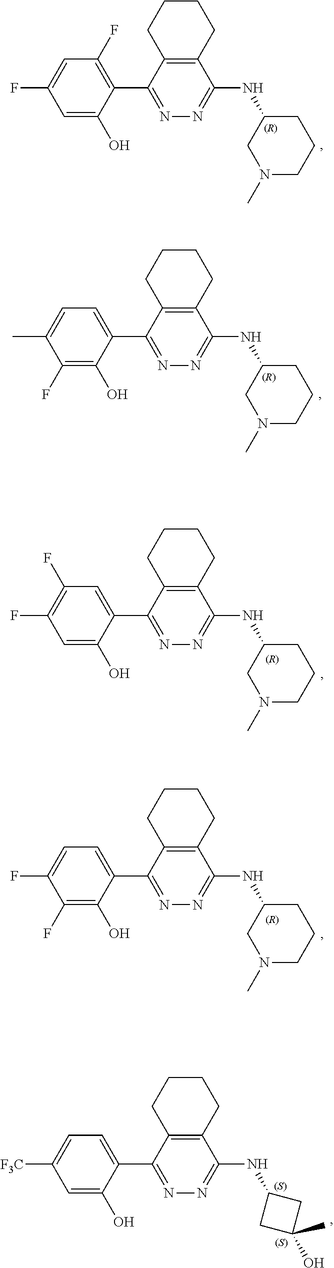

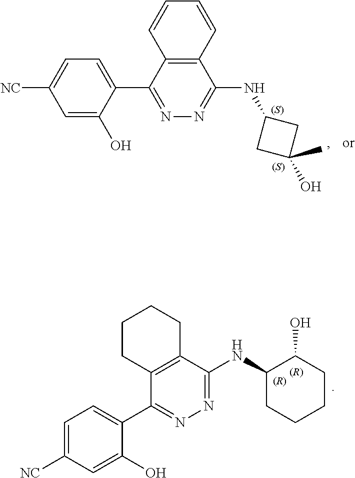

15. A pharmaceutical composition, comprising the compound, or the pharmaceutically acceptable salt, the stereoisomer or the tautomer thereof according to claim 1, and a pharmaceutically acceptable carrier.

16. A method for preventing and/or treating NLRP3 inflammasome-associated diseases, comprising administering prophylactically and/or therapeutically effective amount of the compound, or the pharmaceutically acceptable salt, the stereoisomer or the tautomer thereof according to claim 1 to a subject in need thereof.

17. A method for preventing and/or treating inflammasome-associated diseases, immune diseases, inflammatory diseases, autoimmune diseases, or autoinflammatory diseases, comprising administering prophylactically and/or therapeutically effective amount of the compound, or the pharmaceutically acceptable salt, the stereoisomer or the tautomer thereof according to claim 1 to a subject in need thereof.

18. A pharmaceutical composition, comprising the compound, or the pharmaceutically acceptable salt, the stereoisomer or the tautomer thereof according to claim 14, and a pharmaceutically acceptable carrier.

19. A method for preventing and/or treating NLRP3 inflammasome-associated diseases, comprising administering prophylactically and/or therapeutically effective amount of the compound, or the pharmaceutically acceptable salt, the stereoisomer or the tautomer thereof according to claim 14 to a subject in need thereof.

20. A method for preventing and/or treating inflammasome-associated diseases, immune diseases, inflammatory diseases, autoimmune diseases, or autoinflammatory diseases, comprising administering prophylactically and/or therapeutically effective amount of the compound, or the pharmaceutically acceptable salt, the stereoisomer or the tautomer thereof according to claim 14 to a subject in need thereof.

21. A method for preventing and/or treating NLRP3 inflammasome-associated diseases, comprising administering prophylactically and/or therapeutically effective amount of the pharmaceutical composition according to claim 15 to a subject in need thereof.

22. A method for preventing and/or treating inflammasome-associated diseases, immune diseases, inflammatory diseases, autoimmune diseases, or autoinflammatory diseases, comprising administering prophylactically and/or therapeutically effective amount of the pharmaceutical composition according to claim 15 to a subject in need thereof.

23. A method for preventing and/or treating NLRP3 inflammasome-associated diseases, comprising administering prophylactically and/or therapeutically effective amount of the pharmaceutical composition according to claim 18 to a subject in need thereof.

24. A method for preventing and/or treating inflammasome-associated diseases, immune diseases, inflammatory diseases, autoimmune diseases, or autoinflammatory diseases, comprising administering prophylactically and/or therapeutically effective amount of the pharmaceutical composition according to claim 18 to a subject in need thereof.
